(19) United States
(12) Patent Application Publication
(10) Pub. No.: US 2024/0327813 A1
(43) Pub. Date: Oct. 3, 2024

(54) CRISPR ENZYMES, METHODS, SYSTEMS AND USES THEREOF

(71) Applicant: Beam Therapeutics Inc., Cambridge, MA (US)

(72) Inventors: Bernd ZETSCHE, Cambridge, MA (US); David A. BORN, Cambridge, MA (US); Luis Alberto BARRERA, Cambridge, MA (US)

(21) Appl. No.: 18/736,840

(22) Filed: Jun. 7, 2024

Related U.S. Application Data
(63) Continuation of application No. PCT/US2022/081728, filed on Dec. 16, 2022.
(60) Provisional application No. 63/291,252, filed on Dec. 17, 2021.

(51) Int. Cl. C12N 9/22; C12N 9/78; C12N 15/11; C12N 15/90

(52) U.S. Cl. CPC C12N 9/22; C12N 9/78; C12N 15/11; C12N 15/907; C12Y 305/04004; C07K 2319/00; C12N 2310/20

(57) ABSTRACT

The present invention provides novel systems, methods and compositions for making and using recombinantly engineered novel Cas9 enzymes optimized for human cells, for nucleic acid targeting and manipulation. The present invention is based on the discovery of novel Cas9 enzymes from Streptococcus equinus ATCC 33317, Enterococcus hirae strain F1129E, Streptococcus equinus strain AG46, Staphylococcus simulans strain 19, Streptococcus intermedius B196 strain G1552, Streptococcus sanguinis SK330, Streptococcus sp. C150, Streptococcus oralis subsp. oralis strain RH_1735_08, Streptococcus oralis SK313, Staphylococcus warneri strain 691, Staphylococcus stiuri strain SNUC 2430, Streptococcus gallolyticus strain AM24-4, Lactobacillus kullabergensis strain Biut2, and Streptococcus suis strain LSS83 bacteria that were codon-optimized and recombinantly produced for use in human cells. In some embodiments, novel Cas9 enzymes can be used for base editing. In some embodiments, the novel engineered Cas9 enzymes are used to treat human diseases.

### CROSS-REFERENCE TO RELATED APPLICATIONS

This application is a Continuation application of International Application No. PCT/US2022/081728, filed on Dec. 16, 2022, which claims priority to U.S. Provisional Patent Application Ser. No. 63/291,252 filed Dec. 17, 2021, the contents of each of which are incorporated by reference herein in entirety for all purposes.

### INCORPORATION-BY-REFERENCE OF SEQUENCE LISTING

The contents of the file named “BEM-013W01_SL.xml”, which was created on Nov. 10, 2022 and is 239 kilobytes in size, is hereby incorporated by reference in its entirety.

### BACKGROUND

Enzymes from the prokaryotic Clustered, Regularly Interspaced Short Palindromic Repeats (CRISPR) and CRISPR-associated protein (CRISPR-Cas) systems have been harnessed as reprogrammable and highly specific genome editing tools for use in eukaryotes. Besides genome editing and cleavage, CRISPR-Cas9 can be used to localize effector molecules to specific sites on the genome, allowing genetic and epigenetic regulation and transcriptional modulation through a variety of mechanisms.

However, diverse genomes and genomic targets require a variety of tools for effective genetic engineering, and there remains a need to expand the CRISPR toolbox through the discovery and engineering of novel Cas proteins that can recognize and target diverse sequences.

While CRISPR-Cas9 systems can be used to knock out a gene or modify the expression of a gene, certain kind of gene editing requires precise modifications to the target gene, such as editing a single base within the gene. Such precise modifications remain a challenge and requires a diverse gene editing toolkit to effectuate precise genomic modifications in a wide variety of target genes.

### SUMMARY OF THE INVENTION

The identification of novel Cas9 enzymes with specificity for unique protospacer adjacent motifs (PAM) allows for the expansion of the available tools for gene editing. The present invention provides, among other things, engineered, non-naturally occurring novel Cas9 enzymes isolated from Streptococcus equinus ATCC 33317, Enterococcus hirae strain F1129E, Streptococcus equinus strain AG46, Staphylococcus simulans strain 19, Streptococcus intermedius B196 strain G1552, Streptococcus sanguinis SK330, Streptococcus sp. C150, Streptococcus oralis subsp. oralis strain RH_1735_08, Streptococcus oralis SK313, Staphylococcus warneri strain 691, Staphylococcus stiuri strain SNUC 2430, Streptococcus gallolyticus strain AM24-4, Lactobacillus kullabergensis strain Biut2, and Streptococcus suis strain LSS83.

The present invention is based, in part, on the surprising discovery that novel Cas9 enzymes discovered from different bacteria, which recognize specific PAM sequences can be engineered for expression in eukaryotic cells (e.g., human, plant, etc.). Accordingly, the described Cas9 enzymes and their variants are functional in eukaryotes. The examples provided herewith show use of engineered, non-naturally Cas9 enzymes in human cells with diverse PAM recognition sequences to target various genomic sites. For example, Cas9 engineered from Streptococcus equinus ATCC 33317 recognizes the consensus PAM sequence 5′-NRGNR-3′, Enterococcus hirae strain F1129E recognizes the consensus PAM sequence 5′-NRG-3′, Streptococcus equinus strain AG46, Staphylococcus warneri strain 691, and Staphylococcus sciuri strain SNUC 2430 recognize the consensus PAM sequence 5′-NNGR-3′, Staphylococcus simulans strain 19 recognizes the consensus PAM sequence 5′-NNGRRT-3′, Streptococcus intermedius B196 strain G1552 recognizes the consensus PAM sequence 5′-NNAAAA-3′, Streptococcus sanguinis SK330 recognizes the consensus PAM sequence 5′-NGGNG-3′, Streptococcus sp. C150 recognizes the consensus PAM sequence 5′-NNGNRG-3′, Streptococcus oralis subsp. oralis strain RH_1735_08 recognizes the consensus PAM sequence 5′-NNAAAC-3′, Streptococcus oralis SK313 recognizes the consensus PAM sequence 5′-NNRAAG-3′, Streptococcus gallolyticus strain AM24-4 recognizes the consensus PAM sequence 5′-NNAYAA-3′, Lactobacillus kullabergensis strain Biut2 recognizes the consensus PAM sequence 5′-NNGAAA-3′, and Streptococcus suis strain recognizes the consensus PAM sequence 5′-NNAAA-3′ (H=A, C or T; R=A or G).

In one aspect, an engineered, non-naturally occurring Cas9 protein modified from Streptococcus equinus ATCC 33317 Cas9, Enterococcus hirae strain F1129E Cas9, Streptococcus equinus strain AG46 Cas9, Staphylococcus simulans strain 19 Cas9, Streptococcus intermedius B196 strain G1552, Streptococcus sanguinis SK330 Cas9, Streptococcus sp. C150 Cas9, Streptococcus oralis subsp. oralis strain RH_1735_08 Cas9, Streptococcus oralis SK313 Cas9, Staphylococcus warneri strain 691 Cas9, Staphylococcus sciuri strain SNUC 2430 Cas9, Streptococcus gallolyticus strain AM24-4 Cas9, Lactobacillus kullabergensis strain Biut2 Cas9, and Streptococcus suis strain LSS83 Cas9 is provided herein.

In some embodiments, the Streptococcus equinus ATCC 33317 Cas9 protein has at least 80% sequence identity to

(SEQ ID NO: 1)
MEKSYSIGLDIGTNSVGWSVITDDYKVPAKKMRVLGNTDKKYIKKNLLGALLFDSG
ETAEATRLKRTARRRYTRRKNRLRYLQEIFAKEMAKVDESFFQRLDESFLTDDDKTF
DSHPIFGNKAEEDAYHQKFPTIYHLRKHLADSQEKADLRLIYLALAHMIKFRGHFLY
DNFNDDNFDWRNIDIQKRYEEFIETYDSTLGESYLADISVDAASILEEKVSKTERLENL
LKYYPTEKKTTFFGNLIKLILGQQAKFKAIFNLEDEISLQFATPTYDEDLEELLGKIDN
GDSYSELFVAAQNLYNTILLASFLKTDNKSAKAPLSTSMIERYENHKKDLAKLKDFV
KKNCPDQYHDIFRDKSKNGYAGYIDNGVSQNDFYTFIGKCLEESLKKDKGAQYFLD
KIDRDDFLRKQRTFDNGAFPYQIHLQEMHAILRRQGDYYPFLKENQDKIEKILTFRIP
YYVGPLARKDSRFAWANYRSDEAITPWNFDEVVDKEKSAEKFITRMTLNDLYLPEE
KVLPKHSLIYETFTVYNELTNIKYVNDQGNAIHFDSELKEKIFNQLFKENRKVSKKTLI
DFLNNSEGIYTDKLVGIDEEVKYLNASLGTYHDLKKILESFMDDEINEKIIEDIIQTLTL
FEDIEMKRQRLQKYDDIFTPKQLKELARRNYTGWGRLSYKLINGIRNKENNKTILDY
LKNGNRNFMQLINDDRLSFKQIIIDARKIEKLDNIESVVYNLPGSPAIKKGILQSIKIVD
ELVKVMGHNPDNIVIEMARENQTTNQGKNRSQQRLKRLQDSMSNFKDSSISLKDVD
NSDLQNDRLFLYYIQNGKDMYTGEELDIDHLSDYDIDHIIPQSFIKDNSIDNRVLTSSA
KNRGKSDDVPGRDVVLKMKPFWKKLYDVKLISKRKFDNLTKSEHGGLTESDKAGFI
KRQLVETRQITKYVAQILDGRFNTKRDDNNKVIRDVKVITLKSSLVSQFRKDFGFYK
VREINDYHHAHDAYLNAVVGTAILKKYPKLAPEFVYGEYKKCDVRKLIAKSGDKSEI
GKATAKYFFYSNLMNFFKRVIRYSNGMIVVRPVIEYSKDTGEIAWDKEKDFKTVCK
VLSCPQVNIVKKVEKQSHGLDRGKPKGFYNANPSPKPKKGSKVNLVPIKANLNPKN
YGGYAGISNSYAVLVDATIEKGAKKKLTRIQEFQGISIIDREKYEKNKVEFLKGLGYK
EIYSIITLPKYSLFELADGSRRMLASILSTNNKRGEIHKGNELVLPAKYIPLLYHANRIH
NTFETGHREYVEKHIAEFKEIAEIILEFNNKYVNAKKNSSIIEKALESFDSFSLDEICDSF
VGKLKKNNTKKNSGLFELVSLGSASDFEFLETKVPRYRDYTPSSLLNATLIHQSITGL
YETRIDLSKLGEE

In some embodiments, the Enterococcus hirae strain F1129E Cas9 protein has at least 80% sequence identity to

(SEQ ID NO: 2)
MTKDYTIGLDIGTNSVGWAVLTDDYQLMKRKMSVHGNTEKKKIKKNFWGARLFDE
GQTAEFRRTKRTNRRRLARRKYRLSKLQDLFAEELCKQDDCFFVRLEESFLVPEEKQ
YKPASIFPTLEEEKEYYQKYPTIYHLRQKLVDSTEKEDLRLVYLALAHLLKYRGHFLF
EGDLDTENTSIEESFRVFLEQYSKQSDQPLIVHQPVLTILTDKLSKTKKVEEILKYYPT
EKINSFFAQCLKLIVGNQANFKRIFDLEAEVKLQFSKETYEEDLESLLEKIGDEYLDIF
LQAKKVHDAILLSEIISSTVKHTKAKLSSGMVERYERHKADLAKFKQFVKENVPQKS
TAFFKDTTKNGYAGYIKGKTTQEEFYKFVKKELSGVVGSEPFLEKIDQETFLLKQRT
YTNGVIPHQVHLIELKAIIDQQKQHYPFLEEAGPKIIALFKFRIPYYVGPLAKEQEASSF
AWIERKTAEKIHPWNFSEVVDIEKSAMRFIQRMTKQDTYLPTEKVLPKNSLLYQKYM
IFNELTKVSYKDERGVKQYFSGDEKQQIFKQLFQKERGKITVKKLQNFLYTHYHIEN
AQIFGIEKAFNASYSTYHDFMKLAKTNQKAMQEWLEQPEMEPIFEDIVKILTIFEDRQ
MIKHQLSKYQEVFGEKLLKEFARKHYTGWGRFSAKLIHGIRDRKTNKTILDYLINDD
DVPANRNRNLMQLINDEHLSFKEEIAKATVFSKHKSLVDVIQDLPGSPAIKKGIWQSL
KIVEELIAIIGYKPKNIVIEMARENQKTHRTKPRLKALENGLKQIGSTLLKEQPTDNKA
LQKERLYLYYLQNGRDMYTGEPLEIENLHQYEVDHIIPRSFIVDNSIDNKVLVARKQN
QKKRDDVPKKQIVNEQRIFWNQLKEAKLISPKKYAYLTKIELTPEDKARFIQRQLVET
RQITKHVANILHQSFHQEEEGTDCDGVQIITLKATLTSQFRQTFGLYKVREINPHHHA
HDAYLNGFIANVLLKRYPKLAPEFVYGKYVKYSLARENKATAKKEFYSNILKFFESD
EPFCDENGEIYWEKSHHLPRIKKVLSSHQVNVVKKVEQQKGGFYKETVNSKEKPDK
LIERKNNWDVTKYGGFGSPVIAYAIAFVYAKGKTQKKTRAIEGITIMEQAAFEKDPTT
FLKEKGFPQVTEFIKLPKYTLFEFDNGRRRFLASHKESQKGNPFILSDQLVTLLYHAQ
HYDKITYQESFDYVNTHLSDFSAILTEVLAFAEKYTLADKNIERIQELYEENKYGEIS
MIAQSFLQLLQFNAIGAPADFKFFGVTIPRKRYTSLTEIWDATIIYQSVTGLYETRIRM
GDLWAGEQ

In some embodiments, the Streptococcus equinus strain AG46 Cas9 protein has at least 80% sequence identity to

(SEQ ID NO: 3)
MTNGKILGLDIGVASVGVGIIEAKTGKVIHANSRLFSAANAENNAERRGFRGARRLT
RRKKHRVKRVRDLFEKYDISTDFRNLNLNPYELRVKGLSEQLTNEELFAALRTIAKR
RGISYLDDAEDDSTGSSDYAKSIDENRRLLKSMTPGQIQLERLEKYGQLRGNFTVYD
ENGEAHRLINVESTSDYKNEARKILETQSNYNKQITDEFIEDYIEILTQKRKYYHGPGN
EKSRTDYGRFRTDGTTLENIFGILIGKCSFYPEEYRASKASYTAQEFNFLNDLNNLKV
PTETGKLSTEQKEYLVDFAKNSKTLGASKLLKEIAKLVDGDVKDISGYREDSKGKPD
LHTFEPYRKLKFNLTTVDIDNLSRDILDKLANILTLNTEREGIEDAINRNLPEQFTKEQI
SEIVQIRKSQSSAFNKGWHSFSAKLMNELIPELYATSEEQMTILTRLEKFKATKKSSK
NTKTIDEKEITDEIYNPVVAKSVRQTIKIINAAVKKYGDFDKIVIEMPRDKNADDEKK
FIDKKNKENKKEKDDSLKRAAYLYNGTDKLPDDVFHGNKQLATKIRLWYQQGERC
LYSGKPILIQDLVHNSNNFEIDHILPLSLSFDDSLANKVLVYAWTNQEKGQKTPYQVI
DSMDAAWSFREMKDYVLKQKGIGKKKREYLLTTENIDKIEVKKKFIERNLVDTRYA
SRVVLNSLQTALKELGKDTKVSVVRGQFTSQLRRKWKIDKSRETYHHHAVDALIIAA
SSQLKLWQKHENLMFENYGENQVVNKETGEILSISDDEYKELVFQPPYQGFVNTISS
KAFEDEILFSYQVDSKFNRKVSDATIYSTRKAKLGKDKKEETYVLGKIKDIYSQDGFD
TFIKRYKKDKTQFLMYQKDPLTWENVIEVILRDYPTTKKSEDGKNDVKCNPFEEYRR
ENGLICKYSKKGKGTPIKSLKYYDKKLGNHISITPKESKNDVVLQSLNPWRADLYFN
PDTLKYELMGLKYSDLSFEKGTGKYHISQEKYDEIKEKEGIGQNSEFKFTLYRNDLILI
KDTESGEQEIYRFLSRTMPNVKHYVELKPYDKEKFNGGQELIKSLGEADKVGRCLKG
LSKPGISIYKVRTDVLGNKFFVKKEGDKPKLDFKNNKK

In some embodiments, the Staphylococcus simulans strain 19 Cas9 protein has at least 80% sequence identity to

(SEQ ID NO: 4)
MNNSYILGLDIGITSVGYGIIEYETRDVIDAGVRLFKEANVENNEGRRSKRGARRLKR
RRRHRLQRVKKMLFDYKLLNEDSEISGINPYEARVKGLSEKLSDEEFSAALLHLAKR
RGVHNVSDVEEDTGNELSTKEQIARNNKALEDKYVAELQLERLKEQGEVRGAANRF
KTSDYIKEAKQLLKTQSDYHKIDETFIETYISLLETRRTYYEGPGEGSPFGWKDIKEW
YEMLMGHCTYFPEELRSVKYAYNADLYNALNDLNNLVIARDENEKLEYYEKFQIIE
NVFKQKKKPTLKQIAKEILVNEEDIKGYRVTSTGKPEFTNFKIYHDIKGITSRKEILEN
ADLLNQIAEILTIYQSAEDIQEELAELEPKLTQEEIEQISNLTGYTGTHRLSLKAINLILD
ELWNTSDNQMTIFNRLKLVPKKVDLSQQKEIPTSLVDDFILSPVVKRSFIQSIKVINAII
KKFGLPKDIIIELAREKNSKEAQKFINEMQKRNRQTNERIEKIIKETGKEKAKFLIEKIK
LHDMQEGKCLYSLESIPLEDLLNNPYHYEVDHIIPRSVSFDNSFNNKVLVKQEENSKK
GNRTPFQYLSSSDAKISYETFKKHILNLSKGKGRVSKKKKEYLLEERDINRFSVQKDF
INRNLVDTRYATRELMNLLRSYFRVNDLDVKVKSINGGFTSFLRTKWKFKKERNQG
YKHHAEDALVIANADFIFKEWKKLDTTNKVMENQTVEEQQAENMPGIETDDEYKEI
FVIPRQIQSIKDFKDYKYSHRVDKKPNRELVNDTLYSTRKDDKGNTLIINNIKGLYDK
DNDKLKNLIKKSPEKLLMYHHDPQTYQKLKTIMEQYSNEKNPLYKYHEETGNYLTK
YSKKDNGPIIKKVKYYGKKLNAHLDITNDYSNSQNKIVKLSLKPYRFDVYLDNGGY
KFVTVKNLDVIKKEGFFKIDSNAYEKAKSEKKIDENAVFIASFYNNDLIKIDGELYRIV
GVNNDTRNVVELNMIPITYKEYLENINDKRTPRILKTISQKTYSIEKYSTDILGNLYKV
KSKKKPQMIMKG

In some embodiments, the Streptococcus_intermedius_B196_strain_G1552 Cas9 protein has at least 80% sequence identity to

(SEQ ID NO: 5)
MNGLVLGLDIGIASVGVGILNKETGEIIHVNSRIFPAATADSNVERRGFRQGRRLGRR
KKHRSARLNDLFEEFGFITDFSAVPLNLNPYALRVKGLSEELTNEELFIALKNIIKRRGI
SYLDDASEDGETASNEYGKAVEENRKLLADKTPGQIQLERFEKYGQVRGDFTVVEN
GENHRLINVESTSAYKKEAERILRRQQEFNVRISDEFIEAYLTILTGKRKYYHGPGNEK
SRTDYGRFRTDGTTLDNIFGILIGKCTFYPDEYRAAKASYTAQEFNLLNDLNNLTVPT
ETKKLRPEQKRQIVEYARTAKTLGTPTLLKYIAKLVDGSIDDIKGYRIDKSDKPEMHT
FDAYRKMRTLELVDVDILSRETLDDLAYILTLNTESEGILEALNSKMPGTFTKEQIDE
LIQFRKKNSAVFGKGWHNFSLKLMNELISELYETSEEQMTILTRLGKQRSREISKRTK
YIDEKELTDEIYNPVVAKSVRQAIKIINLATKKYGIFDNIVIEMARESNEDDEKKAIQN
VQKANEDEKKAAMEKAADLYNGKKELPDSIFHGHKELATKIRLWHQQGERCLYTG
KNISIHDLIHNPHQYEIDHILPLSLSFDDGLANKVLVLATANQEKGQRTPFQALDSMD
DAWSYIEFKQYVRNSKSLSNKKKDYLLTEEDISKIEVKQKFIERNLVDTRYSSRVVLN
TLQEFYKTNDFDTKISVVRGQFTSQLRRKWKIEKSRDTYHHHAVDALIIAASSQLRL
WKKQNNPLISYREGQLVDPETGEILSLTDDEYKELVFRPPYDYFVDTLKSKSFEDSILF
SYQVDSKYNRKISDATIYGTRKAQLGKDKQEETYVLGKIKDIYSQKGYEDFIKRYKK
DTTQFLMYHKDPQTFAKVIEEILKTYPDKELNEKGKEIPCNPFEKYRQENGPIRKYSK
KGKGPEIKSLKYYDNKLGNHIDITPVNSQNQVVLQSLKPWRTDVYFNPQTSKYELM
GLKYSDLRFEKGSGSYGISPEKYNKVKAKEGVDEDSEFKFTLYKNDLILIKDTETGEQ
QLFRYGSRNDTSKHYVELKPYEKAKFEGNQQLMNLLGTVAKGGQCLKGINKPNLSI
YKVKTDVLGNKHFIKKEGDQPQLNFKKKI

In some embodiments, the Streptococcus sanguinis SK330 Cas9 protein has at least 80% sequence identity to

(SEQ ID NO: 6)
MENKNYSIGLDIGTNSVGWAVITDDYKVPSKKMKVFGNTDKHFIKKNLIGALLFDEG
ATAEDRRLKRTARRRYTRRKNRLRYLQEIFSEEISKLDSSFFHRLDDSFLVPKDKRGS
KYPIFATLEEEKEYHKKFPTIYHLRKHLADSKEKTDLRLIYLALAHMIKYRGHFLYEE
SFDIKNNDIQKIFNEFISIYDNTFEGSSLSGQNAQVEAIFTDKISKSAKRERVLKLFSDE
KSTSLFSEFLKLIVGNQADFKKHFDLEEKAPLQFSKDTYDEDLENLLGQIGDGFTDLF
LVAKKLYDAILLSGILTVTDPSTKAPLSASMIERYESHQKDLAALKQFIKNNLPKRYN
EVFSDQSKDGYAGYIDGKTTQEAFYKYIKNLLSKFEGADYFLDKIEREDFLRKQRTF
DNGSIPHQIHLQEMNAILRRQGEHYPFLKENREKIEKILTFRIPYYVGPLARGNRDFA
WLTRNSDQAIRPWNFEEIVDKASSAEEFINKMTNYDLYLPEEKVLPKHSLLYETFAV
YNELTKVKFIAEGLRDYQFLDSGQKKQIVNQLFKEKRKVTEKDIIHYLHNVDGYDGI
ELKGIEKQFNASLSTYHDLLKIIKDKEFMDDPKNEEILENIVHTLTIFEDREMIKQRLA
QYDSIFDEKVIKALTRRHYTGWGKLSAKLINGICDKQTGDTILDYLIDDGKINRNFMQ
LINDDGLSFKEIIQKAQVVGKTDDVKQVVQELPGSPAIKKGILQSIKIVDELVKVMGY
APESIVIEMARENQTTARGKKNSQQRYKRIEDSLKNLAPGLDSNILKENPTDNIQLQN
DRLFLYYLQNGKDMYTGKPLDIDQLSSYDIDHIIPQAFIKDDSIDNRVLTSSKDNRGK
SDNVPSLEVVQKRKAFWQQLLDSKLISERKENNLTKAERGGLDERDKVGFIRRQLVE
TRQITKHVAQILDASFNTEVNEKNQKIRTVKIITLKSNLVSNFRKEFELYKVREINDYH
HAHDAYLNAVVAKAILKKYPKLEPEFVYGDYQKYDLKRYISRFKPSKEIEKATEKYF
FYSNLLNFFKEEVHYADGIIVKRENIEYSKDTGEIAWNKEKDFATIKKVLSYPQVNIV
KKTEIQTHGLDRGKPKGLFNSNPSPKPSEDSKENLVPIKQGLDPRKYGGYAGISNSYA
VLVKAIVEKGAKKQQKTILEFQGISILDKINFENNKENYLLKKRYIEILSTITLPKYSLF
EFPDGTRRRLASILSTNNKRGEIHKGNELVLPGKYTTLLYHAKNINKKLEPEHLEYVE
KHRNDFAKLLECVLNFNDKYVGALKNGERIRQAFTDWETVDIEKLCFSFIGPENSKN
AGLFELTSQGSASDFEFLGVKIPRYRDYAPSSLLKATLIHQSITGLYETRIDLSKLGED

In some embodiments, the Streptococcus sp. C150 Cas9 protein has at least 80% sequence identity to

(SEQ ID NO: 7)
MSDLVLGLDIGIGSVGVGILNKVTGEIIHKNSRIFPAAQAENNLERRTNRQGRRLTRR
KKHRRVRLNHLFEESGLITDFTKVSINLNPYQLRVKGLTAELSNEELFIALKNMVKHR
GISYLDDASDDGNSSVGDYAQIVKENSKQLETKTPGQIQLERYQKYGQLRGDFTVEE
DGRKHRLINVFPTSAYHAEALRILQTQQEFNPQITDEFINSYLEILTGKRKYYHGPGNE
KSRTDYGKYTTKKDAQGQYITLNNIFGILIGKCTFYPEEYRAAKASYTAQEFNLLNDL
NNLTVPTETKKLSEEQKYQIITYVKNEKAMGPAKLFKYIAKLLSCDVADIKGYRIDKS
DKAEIHTFEAYRKMKTLETLDVKKMAREELDKLAYVLTLNTEREGIQEALDHEFAD
GTFSQEQVDELVQFRKANSSIFGKGWHSFSVKLMMELIPELYATSEEQMTILTRLGK
QKTTSSSNKTKYIDEKQLTEEIYNPVVAKSVRQAIKIVNAAIKKYGDFDNIVIEMARE
TNEDDEKKAIQKIQKANKAEKDAAMRKAANQYNGKAELPHSVFHGHKQLATKIRL
WHQQGERCLYTGKTISIHDLINNPNQFEIDHILPLSITFDDSLANKVLVYATANQEKG
QRTPYQALDSMDDAWSFRELKAFVRESKALSNKKKEYLLTEEDISKFDVRKKFIERN
LVDTRYASRVVLNALQEHFRTHKIDTKVSVVRGQFTSQLRRHWGIEKTRDTYHHHA
VDALIIAASSQLNLWKKQKNTLVSYSEDQLLDIETGELISDDEYKESVFKAPYQHFVD
TLKSKEFEDSILFSYQVDSKFNRKISDATIYATRKAKLDKEKKEYTYTLGKIKDIYAL
GTKTPSKTGFYKFLDLYKKDKSQFLMYQKDRRTWDEVIEKILEQYRPFKEKDKNGK
EVDENPFEKYRIENGPIRKYSRKGNGPEIKSLKYYDNLLGRFVDITPSESKNPVALLSL
NPWRTDVYYNTETRKYEFLGLKYADLCFEKGGSYGISKVKYNKIREKEGIGKNSEFK
FTLYKNDLILIKDTETNRQQIFRFWSRTGKDNPKSFEKHKLELKPYEKTRFEKGEELK
VLGKVPPSSNRLQKNMQIENLSIYKVRTDVLGNQHIIKNEGDKPKLDF

In some embodiments, the Streptococcus oralis subsp. oralis strain RH_1735_08 Cas9 protein has at least 80% sequence identity to

(SEQ ID NO: 8)
MNGLVLGLDIGIASVGVGILEKNSGKIIHANSRIFPAATADNNVERRKNRQARRLHRR
KKHRGVRLQDIFEDYGLLTDFSKVSINLNPYRLRVDGLDQQLTNEELFIALKNIVKRR
GISYLDDASEDGGTVSSDYGKAVEENRKLLAEKTPGQIQLERFEKYGQVRGDFTVVE
NGEKRRLINVESTSAYRKEAERILRKQQEFNSKITDEFIEDCLKILTGKRKYYHGPGNE
KSRTDYGRFRTDGTTLDNIFGILIGKCTFYPNEYRASKASHTAQEFNLLNDLNNLTVP
TETKKLSEEQKKVIVEYAKEAKTLGASTLLKYIAKMIDASVDQISGYRVDVNNKPEM
HTFEVYRKMQSLETISVGELSRNVLDELAHILTLNTEREGIEEAINTKLKDSFSQDQVL
ELVQFRKNNSSLFSRGWHNFSLKLMMELIPELYETSEEQMTILTRLGKQKSKETSKRT
KYIDEKELTEEIYNPVVAKSVRQAIKIINEATKKYGIFDNIVIEMARENNEEDAKKDYI
KRQKANQDEKNAAMEKAAFQYNGKKELPDNIFHGHKELATKIRLWHQQGEKCLYT
GKNIPISDLIHNQYKYEIDHILPLSLSFDDSLSNKVLVLATANQEKGQRTPFQALDSMD
DGWSYREFKSYVKESKLLGNKKKEYLLTEEDISKIEVKQKFIERNLVDTRYSSRVVL
NALQDFYKAHKFDTTISVVRGQFTSQLRRKWGLEKSRETYHHHAVDALIIAASSQLR
LWKKQNNPLIAYKEGQFVDSETGEILSLSDDEYKELVFKAPYDHFVDTLRSKTFEDSI
LFSYQVDSKYNRKISDATIYATRKAKLDKDKSEETYVLGKIKDIYSQAGYDAFIKIYN
KDKSKFLMYHKDPQTFEKVIEEILRTYPSKELNDKNKEIPCNPFEKYRQENGPIRKYS
KKGNGPEIKCLKYYDNKLGNYIDITPDGSDNQVVLQSIAPWRTDVYYNHKTGKYEF
LGLKYSDLYFEKGTGKYKISKEKYDNIKKIEGVVETSEFKFTLYKNDLILIKDVEKGQ
EQLFRFLSRNNKGKHQVQLKPMNKSDFEKGEKLIDIFGTVPNSTTQCVKGLNKSNISI
FKVKTDVLGKKHIIKKEGDEPKLKF

In some embodiments, the Streptococcus oralis SK313 Cas9 protein has at least 80% sequence identity to

(SEQ ID NO: 9)
MNGLVLGLDIGIASVGVGILEKNSGKIVHASSRIFPAATADNNVERRKNRQARRLHR
RKKHRGARLKDLFEYYGLLTDFSKVSINLNPYRLRVDGLDQQLTNEELFIALKNIVK
RRGISYLDDASEDGGTVSSDYGKAVEENRKLLAEQTPGQIQLERFEKYGQLRGDFTV
VENSEKCRLINVESTSAYKKEAERILRKQQEFNNQITDEFIEDYLKILTGKRKYYHGP
GNEKSRTDYGRFRTDGATLDNIFGILIGKCTFYPNEYRASKASYTAQEFNLLNDLNNL
TVPTETKKLSEEQKKTIIEYAKSAKTLGASTLLKYIAKMIDASVDQIRGYRVDVNNKP
EMHTFEVYRKMQSLETISVGELSRNILDELAHILTLNTEREGIEEAINTKLRDSFSQDQ
VLELVQFRKNNSSLFSKGWHNFSLKLMMELIPELYETSEEQMTILTRLGKQKSKETS
KRTKYIDEKEVTEEIYNPVVAKSVRQAIKIINEATKKHGIFDNIVIEMARENNEEDAK
KDYIKRQKANQDEKYAAMEKAAFQYNGKKELPDNIFHGHKELATKIRLWHQQGEK
CLYTGKSIPISDLIHNQYKYEIDHILPLSLSFDDSLSNKVLVLATANQEKGQRTPFQAL
DSMDDAWSYREFKSYVKESKLLGNKKKEYLLTEEDISKIEVKQKFIERNLVDTRYSS
RVVLNALQDFYKNHNFDTTISVVRGQFTSQLRRKWGLEKSRETYHHHAVDALIIAAS
SQLRLWKKQNNPLIAYKEGQFVDSQTGEIISLTDDEYKELVFKAPYDHFVDTLSSKTF
EDSILFSYQVDSKFNRKISDATIYATRKAKLDKEKKEYTYTLGKIKDIYSLGTKTPSKT
GFYKFLDLYNKDKSQFLMFQKDRKTWDEVIEKIMEQYRPFKEYDKAGKLVDFNPFE
KYRQENGPIRKYSKKGNGPEIKSLKYYDILLGKHKNITPEGSRNTVALLSLNPWRTD
VYYNMETKKYEFLGLKYADLPFEEGGAYGISTETYNELREKEGIGKNSEFKFTLYKN
DLILIKDTETNCQQFFRFWSRTGKDNPKSFEKHKIELKPYEKAKFEKGEELEVLGKVP
PSSNQFQKNMQIENLSIYKVKTDVLGNKHFIKKEGDKPKLKF

In some embodiments, the Staphylococcus warneri strain 691 Cas9 protein has at least 80% sequence identity to

(SEQ ID NO: 10)
MKEKYILGLDLGITSVGYGIINFETKKIIDAGVRLFPEANVDNNEGRRSKRGSRRLKR
RRIHRLDRVKSLLTEYNLINREQIPTSNNPYQIRVKGLSEILSKDELAIALLHLAKRRGI
HNINVSSEDEDASNELSTKEQINRNNKLLKNKYVCEVQLQRLKEGQIRGEKNRFKTT
DILKEIDQLLKVQKDYHNLDIDFINQYKEIVETRREYFEGPGQGSPFGWNGDLKKWY
EMLMGHCTYFPQELRSVKYAYSADLFNALNDLNNLIIQRNDSEKLEYHEKYHIIENV
FKQKKKPTLKQIAKEIGVNPEDIKGYRITKSGTPQFTEFKLYHDLKSIVFDKSILENEAI
LDQIAEILTIYQDEESIKEELNKLPEILNEQDKAEIAKLTGYNVTHRLSLKCIHLINEEL
WQTSRNQMEIFNYLNIKPNKVDLSEQNKIPKDLVDEFILSPVVKRTFIQSINVINKVIE
KYGIPEDIIIELARENNSDDRKKFINNLQKKNEATRKRINEIIGQTGNQNGKRIVEKIRL
HDQQEGKCLYSLESIPLMDLLNNPQNYEVDHIIPRSVAFDNSIHNKVLVKQIENSKKG
NRTPYQYLNSSDANLSYNQFKQHILNLSKSKDRISKKKKDYLLEERDINKFEVQKEFI
NRNLVDTRYATRELTSYLKAYFSANNMDVKVKTINGSFTNHLRKVWRFDKYRNHS
YKHHAEDALIIANADFLFKENKKLQNANKILEKPTIENDTQKVTVEKEEDYNNMFET
PKLVEDIKQYRDYKFSHRVDKKPNRQLIKDTLYSTRMKDEHNYIVQTITDIYGKDNT
NLKKQFNKNPEKFLMYQNDPKTFEKLSIIMKQYSDEKNPLAKYYEETGEYLTKYSK
KNNGPIVKKIKLLGNKVGNHLDVTNKYENSTKKLVKLSIKNYRFDVYLTEKGYKFV
TIAYLNVFKKDNYYYIPKDLYQELKAKKKIKDTDQFIASFYKNDLIKLNGDLYKIIGV
NSDDRNIIELDYYDIKYKDYCEINNIKGEPRIKKTIGKKTESIEKLTTDVLGNLYLHTT
EKAPQLIFKRGL

In some embodiments, the Staphylococcus sciuri strain SNUC 2430 Cas9 protein has at least 80% sequence identity to

(SEQ ID NO: 11)
MKRNYILGLDIGITSVGYGIIDYETRDVIDAGVRLFKEANVENNEGRRSKRGARRLKR
RRRHRLQRVKKLLFDYNLLTDHSELSGINPYEARVKGLSQKLSEVEFSAALLHLAKR
RGVHNVNEVEEDTGNELSTKEQISRNSKALEEKYVAELQLERLKTDGEVRGPNNRF
KTSDYVKEAKQLLKVQKAYHQLDQSFIDTYIDLLETRRTYYEGPGEGSPFGWKDIKE
WYEMLMGHCTYFPEELRSVKYAYNADLYNALNDLNNLVIARDENEKLEYYEKFQII
ENVFKQKKKPTLKQIAKELLVNEEDIKGYRVTSIGKPEFTNFKIYHDIKGITERKEVLE
NAELLDQIAEILTIYQSSEDVQEELANLNSELTQEEIEQISNLKGYTGTHNLSLKAINLI
LDELWHTNDNQMAIFNRLKLVPKKVDLSQQKEIPTTLVDDFILSPVVKRSFIQSIKVIN
AIIKKYGLPNDIIIELAREKNSKDAQKMINEMQKRNRQMNERIEEIIRTTGKENAKYLI
EKIKLHDMQEGKCLYSLESIPLEDLLNNPFNYEVDHIIPRSVSFDNSFNNKVLVKQEE
NSKKGNRTPFQYLSSSDSKISYETFKKHILNLAKGKGRISKTKKEYLLEERDINRFSVQ
KDFINRNLVDTRYATRELMNLLRSYFRVNNLDVKVKSINGGFTSFLRRKWKFKKER
NKGYKHHAEDALIIANADFIFKEWKKLDKAKKVMENQTVEEKQAESMPEIETEQEY
KEIFITPHQIQHIKGFKDYKYSHRVDKKPNRELINDTLYSTRKDDKGNTLIVNNLNGL
YDKDNDKLKKLMNKSPEKLLMYHHDPQTYQKLKLIMEQYGDEKNPLYKYYEETGN
YLTKYSKKDNGPVIKKIKYYGKNLKAHLDITDDYPNSRNKVVKLSVKPYRFDVYLD
NDIYKFVTVKNLDVIKKEDYYEVNSKCYKEAKKLKKISDQAEFIASFYNNDLIKINGE
LYRVIGVNNDLLNRIEVNMINITYREYLENMNDKRPPRIIKTIASKTQSIKKYSTDILG
NLYEVKSKQKPQMIMKG

In some embodiments, the Streptococcus gallolyticus strain AM24-4 Cas9 protein has at least 80% sequence identity to

(SEQ ID NO: 12)
MTNGKILGLDIGIASVGVGIIEAKTGKVVHANSRLFSAANAENNTERRGFRGSRRLNR
RKKHRVKRVRDLFEKHEIVTDFRNLNLSPYELRVKGLTEQLTNEELFAALRTISKRRG
ISYLDDAEDDSTGSTDYAKSIDENRRLLKNKTPGQIQLERLEKYGQLRGNFTVYDEN
GEAHRLINVESTSDYEKEARKILETQADYNKKITAEFIDDYVEILTQKRKYYHGPGNE
KSRTDYGRFRTDGTTLENIFGILIGKCNFYPDEYRASKASYTAQEYNFLNDLNNLKVP
TETGKLPTEQKESLVEFAKNTATLGPSKLLKEIAKILDCKVDEIKGYREDDKGKPDLH
TFEPYRKLKFNLESINIDDLSREVIDKLADILTLNTEREGIEDAIKRNLPNQFTEEQISEII
KVRKSQSTAFNKGWHSFSAKLMNELIPELYATSDEQMTILTRLEKFKVNKKSSKNTK
TIDEKEVTDEIYNPVVAKSVRQTIKIINAAVKKYGDFDKIVIEMPRDKNADDEKKFID
KRNKENKKEKDDALKRAAYLYNSSDKLPDEVFHGNKQLETKIRLWYQQGERCLYS
GKPIPIHDLVHNSNNFEIDHILPLSLSFDDSLANKVLVYDWANQEKGQKTPYQVIDSM
DAAWSFREMKDYVLKQKGLGKKKRDYLLTTENIDKIEVKKKFIERNLIDTRYASRV
VLNSLQSALRELCKDTKVSVIRGQFTSQLRRKWKIDKSRETYHHHAVDALIIAASSQL
KLWEKQDNLMFIDYGNNQVVDKETGEILSVSDDEYKELVFQPPYQGFVNTISSKGFE
DEILFSYQVDSKYNRKVSDATIYSTRKAKIGKDKKEETYVLGKIKDIYSQNGFDTFIK
KYNKDKTQFLMYQKDPLTWENVIEVILRDYPTTKKSEDGKNDVKCNPFEEYRRENG
LICKYSKKGKGTPIKSLKYYDKKLGNCIDITPEESRNKVILQSINPWRADVYFNPETLK
YELMGLKYSDLSFEKGTGKYHISQEKYDAIKEKEGIGKKSEFKFTLYRNDLILIKDTA
SGEQEIYRFLSRTMPNVNHYAELKPYDKEKFDGGQELMEVFGKVANGGQCLKSLNK
SNISIYKVRTDVLGNKYFVKKEGDKPKLNFKNNKK

In some embodiments, the Lactobacillus kullabergensis strain Biut2 Cas9 protein has at least 80% sequence identity to

(SEQ ID NO: 13)
MKRVNEDYILGLDIGTNSCGWAVTDKKNNLLKLRGKTAIGSHLFEEGHTAADRRGF
RTTRRRLKRRKWRLRLLEEIFAEPMAKVDPGFFVRLHQSWVSPLDKDRKKYNAIVFP
TAKEDQAFYKHYATIYHLRDELMTQDRQFDLREIFLAIHHIVKYRGNFLQDTPVKDF
EASKIEVGPILSHINNAFAEKIVEDQDPIELNVANAADIEDVIRGKDAEKTVYKLDKV
KKIAKLLTDSTAKEEKNVAKQIANAIMGYKTQFETILDKEIDKSDKAQWEFKLSDAD
ADDKLDALLPDLDETDQTVVAEIEKLFSAITLSTIVDENKSLSQSMVEKYKKHKKDY
KKLKKYINTLQDQTKAKKLLLAYDLYVNNRHGRLLEAKKTFEDKKKKKALTKDEF
YKIVKDNLDDSDLAHEIQQEIAADNFMPKQRTNSNGVIPFQLHQIELDKIIANQGKYY
PFLAAENPVEDHRKQAPYKLDELVRFRVPYYVGPMITADEQEKTSGKSFAWMVRKE
DGQITPWNFEQKVDRQESANKFIKRMTIKDTYLLSEDVLPANSLLYQRFEVLNELNNI
RINGSRISVDLKQQIFNDLFEEKKTVTEKSLTSYLKQNLHLPTVEIKGLADPTKFNSSL
ASYYHLKSLHVFDKELADPQYQKDFEKIIEYSSIFEDKKIFQDKLHAEFKWLTPEQFK
AISTWRLQGWGRLSRKLLVELHDTNGQNIMEQLWDSQKNFMQIVTEPDFKDAIAKE
NQNVTRANGVEEILADAYTSPANKKAIRQVVKVVADVVKAAGGKKPAQFAIEFTRD
PDKNPQLSHIRGTKLLKAYQETAGELVDQKLTDSLKEAMTSRKLLKDKYFLYFMQA
GRDAYTGQKINIDEVSTNYQIDHILPQSFIKDDSFDNRVLTATPLNAEKSDDVPYKRF
ANNYVSDMKMTVGEMWKHWQKAGIINKHKLGNLLLDPDRLNKFQKSGFINRQLVE
TSQIIKLVSVILQNKYPDAEIITVKAGDNSALRQRLNLYKSRDVNDYHHAIDAYLSIIC
GNFLYQVYPKYRPYFVYGKYKKFSQDPDLQKEVIKHFKGFTFMWPLLQKDNSERKA
PEKIKENNSDRIVFYKHPDIFDKLRKAYNYKYMLVSRETTTENSGLFDVTIYPRGERD
LAKTRKLIPKSNGLDPKIYGGYSGNTDAYMVIVKIDKGKESIYKVIGVPMRALASLN
RAKKQGNYKEELHQVLEPQIMFDKNGKPKRSVKGFRIIKDHVPFKQVVLDGDKKFM
LNSSTYEINAKQLTLTPETMRIVTDNLKKGEDQDQLLVKAYDEILQKVDQYLPLFDV
NKFRNSLHLGRAKFLDLAVNDKKITLTNILNGLHDNLVTPDLKNIGIKTPLGKLQVPS
GIVLSSEAILIFQSPTGLFEKRVRIADL

In some embodiments, the Streptococcus suis strain LSS83 Cas9 protein has at least 80% sequence identity to

(SEQ ID NO: 14)
MSNGKILGLDIGIASVGVGVIDAQTGEIIHASSRIFPSANAANNAERRTFRGSRRLIRR
KKHRIKRLDDLFNDFHINLDGEMSTDNPYVLRVKGLSQKLTVEELYISIKNIMKRRGI
SYLDDAESDNEAGRSDYAKAIERNRQLLTSKTPGEIQLERLEKYGQLRGNFTIIDEEG
QSQQIINVESTSDYVKEVEKILDCQKMYHKFISDEFCDKLIELLREKRKYYVGPGNEK
SRTDYGIYRTDGTTLENLFGILIGKCTFYPDQYRSSRASYTAQEFNFLNDLNNLTVPTE
TKKLSQEQKEFLVNYAKETSVLGAGKILQQIAKLADCKVEDIRGYRLDNKDKPELHT
FETYRAMKGLVPLVDIGVLSREQLDILADILTLNTDFEGIREALKKQLPNVFDEKQVK
GLASFRKSKSQLFAKGWHNLSQKIMLEVIPELYATSDEQMTILTRLGKFEKSSVAEYP
SSINVDEITDEIYNPVVAKSIRQTIKIINASIKKWDEFDQIVIEMPRDRNEDEEKKRIAD
GQKANAKEKADSILRAAELYCAGKVLPDYVYNGHNQLATKIRLWYQQGERCIYTG
QPISIHDLIHNQNQYEIDHILPLSLTFDDSLSNKVLVLATANQEKAQRTPYNYLKSATS
AWSYREFKDYVTKRKGIGKKKCEYLTFEEDINGFEVRSKFIQRNLVDTRYASKVILN
ALQDYFKISGIQTKVSVVRGQFTSQLRHKWGIEKTRETYHHHAVDALIIAASSQLRL
WKKQESPLVVDYQEGRQVDLETGEILELTDEQYKELVYQPPYQGFVNTISSSAFDNEI
LFSYQVDSKVNRKISDATIYATRNAQLGKDKTEGIYVLGKIKDIYTQAGYEAFLKRY
TKDKTSFLMYHKDLDTWEKVIEIILRDYREYDEKGKEIGNPFERYRRENGYVKKYSR
KGNGTAIKSLKYYDNKLGNHIDITPENSRNAVVLQSLKPWRTDVYFNKETGKYEFLG
IKYSDLSFEKGTGEYGISQEKYDSIKIAEGVAKKSIFKFTLYKQDLLFIKDIENNFGKLL
RFTSKNDTSKHYVELKPYDKNKFGTEEPLLPVLGNVAKSGQCIKGLNKSNISIYKVRT
DILGYRHFIKQEGEHPQLKFKK

In some embodiments, the Cas9 protein comprises an amino acid sequence that is at least 85%, at least 90%, at least 92%, at least 95%, at least 96%, at least 97%, at least 98%, or at least 99% identical to SEQ ID NOs: 1, 2, 3, 4, 5, 6, 7, 8, 9, 10, 11, 12, 13 or 14.

In some embodiments, the Cas9 protein further comprises a nuclear localization sequence (NLS) and/or a FLAG, HIS or HA tag.

In some embodiments, the Streptococcus equinus ATCC 33317 Cas9 (Seq2Cas9) has an amino acid sequence at least 80% identical to

(SEQ ID NO: 15)
MPKKKRKVGTMEKSYSIGLAIGTNSVGWSVITDDYKVPAKKMRVLGNTDKKYIKK
NLLGALLFDSGETAEATRLKRTARRRYTRRKNRLRYLQEIFAKEMAKVDESFFQRLD
ESFLTDDDKTFDSHPIFGNKAEEDAYHQKFPTIYHLRKHLADSQEKADLRLIYLALAH
MIKFRGHFLYDNFNDDNFDWRNIDIQKRYEEFIETYDSTLGESYLADISVDAASILEE
KVSKTERLENLLKYYPTEKKTTFFGNLIKLILGQQAKFKAIFNLEDEISLQFATPTYDE
DLEELLGKIDNGDSYSELFVAAQNLYNTILLASFLKTDNKSAKAPLSTSMIERYENHK
KDLAKLKDFVKKNCPDQYHDIFRDKSKNGYAGYIDNGVSQNDFYTFIGKCLEESLK
KDKGAQYFLDKIDRDDFLRKQRTFDNGAFPYQIHLQEMHAILRRQGDYYPFLKENQ
DKIEKILTFRIPYYVGPLARKDSRFAWANYRSDEAITPWNFDEVVDKEKSAEKFITRM
TLNDLYLPEEKVLPKHSLIYETFTVYNELTNIKYVNDQGNAIHFDSELKEKIFNQLFKE
NRKVSKKTLIDFLNNSEGIYTDKLVGIDEEVKYLNASLGTYHDLKKILESFMDDEINE
KIIEDIIQTLTLFEDIEMKRQRLQKYDDIFTPKQLKELARRNYTGWGRLSYKLINGIRN
KENNKTILDYLKNGNRNFMQLINDDRLSFKQIIIDARKIEKLDNIESVVYNLPGSPAIK
KGILQSIKIVDELVKVMGHNPDNIVIEMARENQTTNQGKNRSQQRLKRLQDSMSNFK
DSSISLKDVDNSDLQNDRLFLYYIQNGKDMYTGEELDIDHLSDYDIDHIIPQSFIKDNS
IDNRVLTSSAKNRGKSDDVPGRDVVLKMKPFWKKLYDVKLISKRKFDNLTKSEHGG
LTESDKAGFIKRQLVETRQITKYVAQILDGRFNTKRDDNNKVIRDVKVITLKSSLVSQ
FRKDFGFYKVREINDYHHAHDAYLNAVVGTAILKKYPKLAPEFVYGEYKKCDVRKL
JAKSGDKSEIGKATAKYFFYSNLMNFFKRVIRYSNGMIVVRPVIEYSKDTGEIAWDKE
KDFKTVCKVLSCPQVNIVKKVEKQSHGLDRGKPKGFYNANPSPKPKKGSKVNLVPI
KANLNPKNYGGYAGISNSYAVLVDATIEKGAKKKLTRIQEFQGISIIDREKYEKNKVE
FLKGLGYKEIYSIITLPKYSLFELADGSRRMLASILSTNNKRGEIHKGNELVLPAKYIPL
LYHANRIHNTFETGHREYVEKHIAEFKEIAEIILEFNNKYVNAKKNSSIIEKALESFDSF
SLDEICDSFVGKLKKNNTKKNSGLFELVSLGSASDFEFLETKVPRYRDYTPSSLLNAT
LIHQSITGLYETRIDLSKLGEEKRPAATKKAGQAKKKKGSYPYDVPDYAYPYDVPDYA
YPYDVPDYA

(D10A mutation, bold underlined italics)

In some embodiments, the Enterococcus hirae strain F1129E Cas9 (EhiCas9) has an amino acid sequence at least 80% identical to

(SEQ ID NO: 16)
MPKKKRKVGTMTKDYTIGLAIGTNSVGWAVLTDDYQLMKRKMSVHGNTEKKKIK
KNFWGARLFDEGQTAEFRRTKRTNRRRLARRKYRLSKLQDLFAEELCKQDDCFFVR
LEESFLVPEEKQYKPASIFPTLEEEKEYYQKYPTIYHLRQKLVDSTEKEDLRLVYLAL
AHLLKYRGHFLFEGDLDTENTSIEESFRVFLEQYSKQSDQPLIVHQPVLTILTDKLSKT
KKVEEILKYYPTEKINSFFAQCLKLIVGNQANFKRIFDLEAEVKLQFSKETYEEDLESL
LEKIGDEYLDIFLQAKKVHDAILLSEIISSTVKHTKAKLSSGMVERYERHKADLAKFK
QFVKENVPQKSTAFFKDTTKNGYAGYIKGKTTQEEFYKFVKKELSGVVGSEPFLEKI
DQETFLLKQRTYTNGVIPHQVHLIELKAIIDQQKQHYPFLEEAGPKIIALFKFRIPYYV
GPLAKEQEASSFAWIERKTAEKIHPWNFSEVVDIEKSAMRFIQRMTKQDTYLPTEKV
LPKNSLLYQKYMIFNELTKVSYKDERGVKQYFSGDEKQQIFKQLFQKERGKITVKKL
QNFLYTHYHIENAQIFGIEKAFNASYSTYHDFMKLAKTNQKAMQEWLEQPEMEPIFE
DIVKILTIFEDRQMIKHQLSKYQEVFGEKLLKEFARKHYTGWGRFSAKLIHGIRDRKT
NKTILDYLINDDDVPANRNRNLMQLINDEHLSFKEEIAKATVFSKHKSLVDVIQDLPG
SPAIKKGIWQSLKIVEELIAIIGYKPKNIVIEMARENQKTHRTKPRLKALENGLKQIGST
LLKEQPTDNKALQKERLYLYYLQNGRDMYTGEPLEIENLHQYEVDHIIPRSFIVDNSI
DNKVLVARKQNQKKRDDVPKKQIVNEQRIFWNQLKEAKLISPKKYAYLTKIELTPED
KARFIQRQLVETRQITKHVANILHQSFHQEEEGTDCDGVQIITLKATLTSQFRQTFGLY
KVREINPHHHAHDAYLNGFIANVLLKRYPKLAPEFVYGKYVKYSLARENKATAKKE
FYSNILKFFESDEPFCDENGEIYWEKSHHLPRIKKVLSSHQVNVVKKVEQQKGGFYK
ETVNSKEKPDKLIERKNNWDVTKYGGFGSPVIAYAIAFVYAKGKTQKKTRAIEGITI
MEQAAFEKDPTTFLKEKGFPQVTEFIKLPKYTLFEFDNGRRRFLASHKESQKGNPFIL
SDQLVTLLYHAQHYDKITYQESFDYVNTHLSDFSAILTEVLAFAEKYTLADKNIERIQ
ELYEENKYGEISMIAQSFLQLLQFNAIGAPADFKFFGVTIPRKRYTSLTEIWDATIIYQS
VTGLYETRIRMGDLWAGEQKRPAATKKAGQAKKKKGSYPYDVPDYAYPYDVPDYA
YPYDVPDYA

(D10A mutation, bold underlined italics)

In some embodiments, the Streptococcus equinus strain AG46 (SeqCas9) has an amino acid sequence at least 80% identical to

(SEQ ID NO: 17)
MPKKKRKVGTMTNGKILGLAIGVASVGVGIIEAKTGKVIHANSRLFSAANAENNAE
RRGFRGARRLTRRKKHRVKRVRDLFEKYDISTDFRNLNLNPYELRVKGLSEQLTNEE
LFAALRTIAKRRGISYLDDAEDDSTGSSDYAKSIDENRRLLKSMTPGQIQLERLEKYG
QLRGNFTVYDENGEAHRLINVESTSDYKNEARKILETQSNYNKQITDEFIEDYIEILTQ
KRKYYHGPGNEKSRTDYGRFRTDGTTLENIFGILIGKCSFYPEEYRASKASYTAQEFN
FLNDLNNLKVPTETGKLSTEQKEYLVDFAKNSKTLGASKLLKEIAKLVDGDVKDISG
YREDSKGKPDLHTFEPYRKLKFNLTTVDIDNLSRDILDKLANILTLNTEREGIEDAINR
NLPEQFTKEQISEIVQIRKSQSSAFNKGWHSFSAKLMNELIPELYATSEEQMTILTRLE
KFKATKKSSKNTKTIDEKEITDEIYNPVVAKSVRQTIKIINAAVKKYGDFDKIVIEMPR
DKNADDEKKFIDKKNKENKKEKDDSLKRAAYLYNGTDKLPDDVFHGNKQLATKIR
LWYQQGERCLYSGKPILIQDLVHNSNNFEIDHILPLSLSFDDSLANKVLVYAWTNQE
KGQKTPYQVIDSMDAAWSFREMKDYVLKQKGIGKKKREYLLTTENIDKIEVKKKFIE
RNLVDTRYASRVVLNSLQTALKELGKDTKVSVVRGQFTSQLRRKWKIDKSRETYHH
HAVDALIIAASSQLKLWQKHENLMFENYGENQVVNKETGEILSISDDEYKELVFQPP
YQGFVNTISSKAFEDEILFSYQVDSKFNRKVSDATIYSTRKAKLGKDKKEETYVLGKI
KDIYSQDGFDTFIKRYKKDKTQFLMYQKDPLTWENVIEVILRDYPTTKKSEDGKNDV
KCNPFEEYRRENGLICKYSKKGKGTPIKSLKYYDKKLGNHISITPKESKNDVVLQSLN
PWRADLYFNPDTLKYELMGLKYSDLSFEKGTGKYHISQEKYDEIKEKEGIGQNSEFK
FTLYRNDLILIKDTESGEQEIYRFLSRTMPNVKHYVELKPYDKEKFNGGQELIKSLGE
ADKVGRCLKGLSKPGISIYKVRTDVLGNKFFVKKEGDKPKLDFKNNKKKRPAATK
KAGQAKKKKGSYPYDVPDYAYPYDVPDYAYPYDVPDYA

In some embodiments, the Staphylococcus simulans strain 19 (SsiCas9) has an amino acid sequence at least 80% identical to

(SEQ ID NO: 18)
MPKKKRKVGTMNNSYILGLAIGITSVGYGIIEYETRDVIDAGVRLFKEANVENNEGR
RSKRGARRLKRRRRHRLQRVKKMLFDYKLLNEDSEISGINPYEARVKGLSEKLSDEE
FSAALLHLAKRRGVHNVSDVEEDTGNELSTKEQIARNNKALEDKYVAELQLERLKE
QGEVRGAANRFKTSDYIKEAKQLLKTQSDYHKIDETFIETYISLLETRRTYYEGPGEG
SPFGWKDIKEWYEMLMGHCTYFPEELRSVKYAYNADLYNALNDLNNLVIARDENE
KLEYYEKFQIIENVFKQKKKPTLKQIAKEILVNEEDIKGYRVTSTGKPEFTNFKIYHDI
KGITSRKEILENADLLNQIAEILTIYQSAEDIQEELAELEPKLTQEEIEQISNLTGYTGTH
RLSLKAINLILDELWNTSDNQMTIFNRLKLVPKKVDLSQQKEIPTSLVDDFILSPVVKR
SFIQSIKVINAIIKKFGLPKDIIIELAREKNSKEAQKFINEMQKRNRQTNERIEKIIKETG
KEKAKFLIEKIKLHDMQEGKCLYSLESIPLEDLLNNPYHYEVDHIIPRSVSFDNSFNNK
VLVKQEENSKKGNRTPFQYLSSSDAKISYETFKKHILNLSKGKGRVSKKKKEYLLEE
RDINRFSVQKDFINRNLVDTRYATRELMNLLRSYFRVNDLDVKVKSINGGFTSFLRT
KWKFKKERNQGYKHHAEDALVIANADFIFKEWKKLDTTNKVMENQTVEEQQAEN
MPGIETDDEYKEIFVIPRQIQSIKDFKDYKYSHRVDKKPNRELVNDTLYSTRKDDKGN
TLIINNIKGLYDKDNDKLKNLIKKSPEKLLMYHHDPQTYQKLKTIMEQYSNEKNPLY
KYHEETGNYLTKYSKKDNGPIIKKVKYYGKKLNAHLDITNDYSNSQNKIVKLSLKPY
RFDVYLDNGGYKFVTVKNLDVIKKEGFFKIDSNAYEKAKSEKKIDENAVFIASFYNN
DLIKIDGELYRIVGVNNDTRNVVELNMIPITYKEYLENINDKRTPRILKTISQKTYSIEK
YSTDILGNLYKVKSKKKPQMIMKGKRPAATKKAGQAKKKKGSYPYDVPDYAYPYD
VPDYAYPYDVPDYA

In some embodiments, the Streptococcus intermedius B196 strain G1552 (SinCas9) has an amino acid sequence at least 80% identical to

(SEQ ID NO: 19)
MPKKKRKVGTMNGLVLGLAIGIASVGVGILNKETGEIIHVNSRIFPAATADSNVERR
GFRQGRRLGRRKKHRSARLNDLFEEFGFITDFSAVPLNLNPYALRVKGLSEELTNEEL
FIALKNIIKRRGISYLDDASEDGETASNEYGKAVEENRKLLADKTPGQIQLERFEKYG
QVRGDFTVVENGENHRLINVESTSAYKKEAERILRRQQEFNVRISDEFIEAYLTILTGK
RKYYHGPGNEKSRTDYGRFRTDGTTLDNIFGILIGKCTFYPDEYRAAKASYTAQEFN
LLNDLNNLTVPTETKKLRPEQKRQIVEYARTAKTLGTPTLLKYIAKLVDGSIDDIKGY
RIDKSDKPEMHTFDAYRKMRTLELVDVDILSRETLDDLAYILTLNTESEGILEALNSK
MPGTFTKEQIDELIQFRKKNSAVFGKGWHNFSLKLMNELISELYETSEEQMTILTRLG
KQRSREISKRTKYIDEKELTDEIYNPVVAKSVRQAIKIINLATKKYGIFDNIVIEMARES
NEDDEKKAIQNVQKANEDEKKAAMEKAADLYNGKKELPDSIFHGHKELATKIRLW
HQQGERCLYTGKNISIHDLIHNPHQYEIDHILPLSLSFDDGLANKVLVLATANQEKGQ
RTPFQALDSMDDAWSYIEFKQYVRNSKSLSNKKKDYLLTEEDISKIEVKQKFIERNLV
DTRYSSRVVLNTLQEFYKTNDFDTKISVVRGQFTSQLRRKWKIEKSRDTYHHHAVD
ALIIAASSQLRLWKKQNNPLISYREGQLVDPETGEILSLTDDEYKELVFRPPYDYFVD
TLKSKSFEDSILFSYQVDSKYNRKISDATIYGTRKAQLGKDKQEETYVLGKIKDIYSQ
KGYEDFIKRYKKDTTQFLMYHKDPQTFAKVIEEILKTYPDKELNEKGKEIPCNPFEKY
RQENGPIRKYSKKGKGPEIKSLKYYDNKLGNHIDITPVNSQNQVVLQSLKPWRTDVY
FNPQTSKYELMGLKYSDLRFEKGSGSYGISPEKYNKVKAKEGVDEDSEFKFTLYKND
LILIKDTETGEQQLFRYGSRNDTSKHYVELKPYEKAKFEGNQQLMNLLGTVAKGGQ
CLKGINKPNLSIYKVKTDVLGNKHFIKKEGDQPQLNFKKKIKRPAATKKAGQAKKK
KGSYPYDVPDYAYPYDVPDYAYPYDVPDYA

In some embodiments, the Streptococcus sanguinis SK330 Cas9 has an amino acid sequence at least 80% identical to

(SEQ ID NO: 20)
MPKKKRKVGTMENKNYSIGLAIGTNSVGWAVITDDYKVPSKKMKVFGNTDKHFIK
KNLIGALLFDEGATAEDRRLKRTARRRYTRRKNRLRYLQEIFSEEISKLDSSFFHRLD
DSFLVPKDKRGSKYPIFATLEEEKEYHKKFPTIYHLRKHLADSKEKTDLRLIYLALAH
MIKYRGHFLYEESFDIKNNDIQKIFNEFISIYDNTFEGSSLSGQNAQVEAIFTDKISKSA
KRERVLKLFSDEKSTSLFSEFLKLIVGNQADFKKHFDLEEKAPLQFSKDTYDEDLENL
LGQIGDGFTDLFLVAKKLYDAILLSGILTVTDPSTKAPLSASMIERYESHQKDLAALK
QFIKNNLPKRYNEVFSDQSKDGYAGYIDGKTTQEAFYKYIKNLLSKFEGADYFLDKI
EREDFLRKQRTFDNGSIPHQIHLQEMNAILRRQGEHYPFLKENREKIEKILTFRIPYYV
GPLARGNRDFAWLTRNSDQAIRPWNFEEIVDKASSAEEFINKMTNYDLYLPEEKVLP
KHSLLYETFAVYNELTKVKFIAEGLRDYQFLDSGQKKQIVNQLFKEKRKVTEKDIIH
YLHNVDGYDGIELKGIEKQFNASLSTYHDLLKIIKDKEFMDDPKNEEILENIVHTLTIF
EDREMIKQRLAQYDSIFDEKVIKALTRRHYTGWGKLSAKLINGICDKQTGDTILDYLI
DDGKINRNFMQLINDDGLSFKEIIQKAQVVGKTDDVKQVVQELPGSPAIKKGILQSIK
IVDELVKVMGYAPESIVIEMARENQTTARGKKNSQQRYKRIEDSLKNLAPGLDSNIL
KENPTDNIQLQNDRLFLYYLQNGKDMYTGKPLDIDQLSSYDIDHIIPQAFIKDDSIDNR
VLTSSKDNRGKSDNVPSLEVVQKRKAFWQQLLDSKLISERKENNLTKAERGGLDER
DKVGFIRRQLVETRQITKHVAQILDASFNTEVNEKNQKIRTVKIITLKSNLVSNFRKEF
ELYKVREINDYHHAHDAYLNAVVAKAILKKYPKLEPEFVYGDYQKYDLKRYISRFK
PSKEIEKATEKYFFYSNLLNFFKEEVHYADGIIVKRENIEYSKDTGEIAWNKEKDFATI
KKVLSYPQVNIVKKTEIQTHGLDRGKPKGLFNSNPSPKPSEDSKENLVPIKQGLDPRK
YGGYAGISNSYAVLVKAIVEKGAKKQQKTILEFQGISILDKINFENNKENYLLKKRYI
EILSTITLPKYSLFEFPDGTRRRLASILSTNNKRGEIHKGNELVLPGKYTTLLYHAKNIN
KKLEPEHLEYVEKHRNDFAKLLECVLNFNDKYVGALKNGERIRQAFTDWETVDIEK
LCFSFIGPENSKNAGLFELTSQGSASDFEFLGVKIPRYRDYAPSSLLKATLIHQSITGLY
ETRIDLSKLGEDKRPAATKKAGQAKKKKGSYPYDVPDYAYPYDVPDYAYPYDVPDYA

In some embodiments, the Streptococcus sp. C150 Cas9 has an amino acid sequence at least 80% identical to

(SEQ ID NO: 21)
MPKKKRKVGTMSDLVLGLAIGIGSVGVGILNKVTGEIIHKNSRIFPAAQAENNLERR
TNRQGRRLTRRKKHRRVRLNHLFEESGLITDFTKVSINLNPYQLRVKGLTAELSNEEL
FIALKNMVKHRGISYLDDASDDGNSSVGDYAQIVKENSKQLETKTPGQIQLERYQKY
GQLRGDFTVEEDGRKHRLINVFPTSAYHAEALRILQTQQEFNPQITDEFINSYLEILTG
KRKYYHGPGNEKSRTDYGKYTTKKDAQGQYITLNNIFGILIGKCTFYPEEYRAAKAS
YTAQEFNLLNDLNNLTVPTETKKLSEEQKYQIITYVKNEKAMGPAKLFKYIAKLLSC
DVADIKGYRIDKSDKAEIHTFEAYRKMKTLETLDVKKMAREELDKLAYVLTLNTER
EGIQEALDHEFADGTFSQEQVDELVQFRKANSSIFGKGWHSFSVKLMMELIPELYAT
SEEQMTILTRLGKQKTTSSSNKTKYIDEKQLTEEIYNPVVAKSVRQAIKIVNAAIKKY
GDFDNIVIEMARETNEDDEKKAIQKIQKANKAEKDAAMRKAANQYNGKAELPHSVF
HGHKQLATKIRLWHQQGERCLYTGKTISIHDLINNPNQFEIDHILPLSITFDDSLANKV
LVYATANQEKGQRTPYQALDSMDDAWSFRELKAFVRESKALSNKKKEYLLTEEDIS
KFDVRKKFIERNLVDTRYASRVVLNALQEHFRTHKIDTKVSVVRGQFTSQLRRHWGI
EKTRDTYHHHAVDALIIAASSQLNLWKKQKNTLVSYSEDQLLDIETGELISDDEYKES
VFKAPYQHFVDTLKSKEFEDSILFSYQVDSKFNRKISDATIYATRKAKLDKEKKEYTY
TLGKIKDIYALGTKTPSKTGFYKFLDLYKKDKSQFLMYQKDRRTWDEVIEKILEQYR
PFKEKDKNGKEVDENPFEKYRIENGPIRKYSRKGNGPEIKSLKYYDNLLGRFVDITPS
ESKNPVALLSLNPWRTDVYYNTETRKYEFLGLKYADLCFEKGGSYGISKVKYNKIRE
KEGIGKNSEFKFTLYKNDLILIKDTETNRQQIFRFWSRTGKDNPKSFEKHKLELKPYE
KTRFEKGEELKVLGKVPPSSNRLQKNMQIENLSIYKVRTDVLGNQHIIKNEGDKPKL
DFKRPAATKKAGQAKKKKGSYPYDVPDYAYPYDVPDYAYPYDVPDYA

In some embodiments, the Streptococcus oralis subsp. oralis strain RH_1735_08 Cas9 has an amino acid sequence at least 80% identical to

(SEQ ID NO: 22)
MPKKKRKVGTMNGLVLGLAIGIASVGVGILEKNSGKIIHANSRIFPAATADNNVERR
KNRQARRLHRRKKHRGVRLQDIFEDYGLLTDFSKVSINLNPYRLRVDGLDQQLTNEE
LFIALKNIVKRRGISYLDDASEDGGTVSSDYGKAVEENRKLLAEKTPGQIQLERFEKY
GQVRGDFTVVENGEKRRLINVESTSAYRKEAERILRKQQEFNSKITDEFIEDCLKILTG
KRKYYHGPGNEKSRTDYGRFRTDGTTLDNIFGILIGKCTFYPNEYRASKASHTAQEF
NLLNDLNNLTVPTETKKLSEEQKKVIVEYAKEAKTLGASTLLKYIAKMIDASVDQIS
GYRVDVNNKPEMHTFEVYRKMQSLETISVGELSRNVLDELAHILTLNTEREGIEEAIN
TKLKDSFSQDQVLELVQFRKNNSSLFSRGWHNFSLKLMMELIPELYETSEEQMTILTR
LGKQKSKETSKRTKYIDEKELTEEIYNPVVAKSVRQAIKIINEATKKYGIFDNIVIEMA
RENNEEDAKKDYIKRQKANQDEKNAAMEKAAFQYNGKKELPDNIFHGHKELATKI
RLWHQQGEKCLYTGKNIPISDLIHNQYKYEIDHILPLSLSFDDSLSNKVLVLATANQE
KGQRTPFQALDSMDDGWSYREFKSYVKESKLLGNKKKEYLLTEEDISKIEVKQKFIE
RNLVDTRYSSRVVLNALQDFYKAHKFDTTISVVRGQFTSQLRRKWGLEKSRETYHH
HAVDALIIAASSQLRLWKKQNNPLIAYKEGQFVDSETGEILSLSDDEYKELVFKAPYD
HFVDTLRSKTFEDSILFSYQVDSKYNRKISDATIYATRKAKLDKDKSEETYVLGKIKD
IYSQAGYDAFIKIYNKDKSKFLMYHKDPQTFEKVIEEILRTYPSKELNDKNKEIPCNPF
EKYRQENGPIRKYSKKGNGPEIKCLKYYDNKLGNYIDITPDGSDNQVVLQSIAPWRT
DVYYNHKTGKYEFLGLKYSDLYFEKGTGKYKISKEKYDNIKKIEGVVETSEFKFTLY
KNDLILIKDVEKGQEQLFRFLSRNNKGKHQVQLKPMNKSDFEKGEKLIDIFGTVPNST
TQCVKGLNKSNISIFKVKTDVLGKKHIIKKEGDEPKLKFKRPAATKKAGQAKKKKG
SYPYDVPDYAYPYDVPDYAYPYDVPDYA

In some embodiments, the Streptococcus oralis SK313 Cas9 has an amino acid sequence at least 80% identical to

(SEQ ID NO: 23)
MPKKKRKVGTMNGLVLGLAIGIASVGVGILEKNSGKIVHASSRIFPAATADNNVERR
KNRQARRLHRRKKHRGARLKDLFEYYGLLTDFSKVSINLNPYRLRVDGLDQQLTNE
ELFIALKNIVKRRGISYLDDASEDGGTVSSDYGKAVEENRKLLAEQTPGQIQLERFEK
YGQLRGDFTVVENSEKCRLINVESTSAYKKEAERILRKQQEFNNQITDEFIEDYLKILT
GKRKYYHGPGNEKSRTDYGRFRTDGATLDNIFGILIGKCTFYPNEYRASKASYTAQE
FNLLNDLNNLTVPTETKKLSEEQKKTIIEYAKSAKTLGASTLLKYIAKMIDASVDQIR
GYRVDVNNKPEMHTFEVYRKMQSLETISVGELSRNILDELAHILTLNTEREGIEEAIN
TKLRDSFSQDQVLELVQFRKNNSSLFSKGWHNFSLKLMMELIPELYETSEEQMTILTR
LGKQKSKETSKRTKYIDEKEVTEEIYNPVVAKSVRQAIKIINEATKKHGIFDNIVIEMA
RENNEEDAKKDYIKRQKANQDEKYAAMEKAAFQYNGKKELPDNIFHGHKELATKI
RLWHQQGEKCLYTGKSIPISDLIHNQYKYEIDHILPLSLSFDDSLSNKVLVLATANQE
KGQRTPFQALDSMDDAWSYREFKSYVKESKLLGNKKKEYLLTEEDISKIEVKQKFIE
RNLVDTRYSSRVVLNALQDFYKNHNFDTTISVVRGQFTSQLRRKWGLEKSRETYHH
HAVDALIIAASSQLRLWKKQNNPLIAYKEGQFVDSQTGEIISLTDDEYKELVFKAPYD
HFVDTLSSKTFEDSILFSYQVDSKFNRKISDATIYATRKAKLDKEKKEYTYTLGKIKDI
YSLGTKTPSKTGFYKFLDLYNKDKSQFLMFQKDRKTWDEVIEKIMEQYRPFKEYDK
AGKLVDFNPFEKYRQENGPIRKYSKKGNGPEIKSLKYYDILLGKHKNITPEGSRNTVA
LLSLNPWRTDVYYNMETKKYEFLGLKYADLPFEEGGAYGISTETYNELREKEGIGKN
SEFKFTLYKNDLILIKDTETNCQQFFRFWSRTGKDNPKSFEKHKIELKPYEKAKFEKG
EELEVLGKVPPSSNQFQKNMQIENLSIYKVKTDVLGNKHFIKKEGDKPKLKFKRPAA
TKKAGQAKKKKGSYPYDVPDYAYPYDVPDYAYPYDVPDYA

In some embodiments, the Streptococcus oralis SK313 Cas9 has an amino acid sequence at least 80% identical to

(SEQ ID NO: 24)
MPKKKRKVGTMKEKYILGLALGITSVGYGIINFETKKIIDAGVRLFPEANVDNNEGR
RSKRGSRRLKRRRIHRLDRVKSLLTEYNLINREQIPTSNNPYQIRVKGLSEILSKDELAI
ALLHLAKRRGIHNINVSSEDEDASNELSTKEQINRNNKLLKNKYVCEVQLQRLKEGQ
IRGEKNRFKTTDILKEIDQLLKVQKDYHNLDIDFINQYKEIVETRREYFEGPGQGSPFG
WNGDLKKWYEMLMGHCTYFPQELRSVKYAYSADLFNALNDLNNLIIQRNDSEKLE
YHEKYHIIENVFKQKKKPTLKQIAKEIGVNPEDIKGYRITKSGTPQFTEFKLYHDLKSI
VFDKSILENEAILDQIAEILTIYQDEESIKEELNKLPEILNEQDKAEIAKLTGYNVTHRL
SLKCIHLINEELWQTSRNQMEIFNYLNIKPNKVDLSEQNKIPKDLVDEFILSPVVKRTFI
QSINVINKVIEKYGIPEDIIIELARENNSDDRKKFINNLQKKNEATRKRINEIIGQTGNQ
NGKRIVEKIRLHDQQEGKCLYSLESIPLMDLLNNPQNYEVDHIIPRSVAFDNSIHNKV
LVKQIENSKKGNRTPYQYLNSSDANLSYNQFKQHILNLSKSKDRISKKKKDYLLEER
DINKFEVQKEFINRNLVDTRYATRELTSYLKAYFSANNMDVKVKTINGSFTNHLRKV
WRFDKYRNHSYKHHAEDALIIANADFLFKENKKLQNANKILEKPTIENDTQKVTVEK
EEDYNNMFETPKLVEDIKQYRDYKFSHRVDKKPNRQLIKDTLYSTRMKDEHNYIVQ
TITDIYGKDNTNLKKQFNKNPEKFLMYQNDPKTFEKLSIIMKQYSDEKNPLAKYYEE
TGEYLTKYSKKNNGPIVKKIKLLGNKVGNHLDVTNKYENSTKKLVKLSIKNYRFDV
YLTEKGYKFVTIAYLNVFKKDNYYYIPKDLYQELKAKKKIKDTDQFIASFYKNDLIK
LNGDLYKIIGVNSDDRNIIELDYYDIKYKDYCEINNIKGEPRIKKTIGKKTESIEKLTTD
VLGNLYLHTTEKAPQLIFKRGLKRPAATKKAGQAKKKKGSYPYDVPDYAYPYDVPD
YAYPYDVPDYA

In some embodiments, the Staphylococcus warneri strain 691 Cas9 has an amino acid sequence at least 80% identical to

(SEQ ID NO: 25)
MPKKKRKVGTMKRNYILGLAIGITSVGYGIIDYETRDVIDAGVRLFKEANVENNEGR
RSKRGARRLKRRRRHRLQRVKKLLFDYNLLTDHSELSGINPYEARVKGLSQKLSEVE
FSAALLHLAKRRGVHNVNEVEEDTGNELSTKEQISRNSKALEEKYVAELQLERLKTD
GEVRGPNNRFKTSDYVKEAKQLLKVQKAYHQLDQSFIDTYIDLLETRRTYYEGPGEG
SPFGWKDIKEWYEMLMGHCTYFPEELRSVKYAYNADLYNALNDLNNLVIARDENE
KLEYYEKFQIIENVFKQKKKPTLKQIAKELLVNEEDIKGYRVTSIGKPEFTNFKIYHDI
KGITERKEVLENAELLDQIAEILTIYQSSEDVQEELANLNSELTQEEIEQISNLKGYTGT
HNLSLKAINLILDELWHTNDNQMAIFNRLKLVPKKVDLSQQKEIPTTLVDDFILSPVV
KRSFIQSIKVINAIIKKYGLPNDIIIELAREKNSKDAQKMINEMQKRNRQMNERIEEIIR
TTGKENAKYLIEKIKLHDMQEGKCLYSLESIPLEDLLNNPFNYEVDHIIPRSVSFDNSF
NNKVLVKQEENSKKGNRTPFQYLSSSDSKISYETFKKHILNLAKGKGRISKTKKEYLL
EERDINRFSVQKDFINRNLVDTRYATRELMNLLRSYFRVNNLDVKVKSINGGFTSFL
RRKWKFKKERNKGYKHHAEDALIIANADFIFKEWKKLDKAKKVMENQTVEEKQAE
SMPEIETEQEYKEIFITPHQIQHIKGFKDYKYSHRVDKKPNRELINDTLYSTRKDDKGN
TLIVNNLNGLYDKDNDKLKKLMNKSPEKLLMYHHDPQTYQKLKLIMEQYGDEKNP
LYKYYEETGNYLTKYSKKDNGPVIKKIKYYGKNLKAHLDITDDYPNSRNKVVKLSV
KPYRFDVYLDNDIYKFVTVKNLDVIKKEDYYEVNSKCYKEAKKLKKISDQAEFIASF
YNNDLIKINGELYRVIGVNNDLLNRIEVNMINITYREYLENMNDKRPPRIIKTIASKTQ
SIKKYSTDILGNLYEVKSKQKPQMIMKGKRPAATKKAGQAKKKKGSYPYDVPDYAY
PYDVPDYAYPYDVPDYA

In some embodiments, the Streptococcus gallolyticus strain AM24-4 Cas9 has an amino acid sequence at least 80% identical to

(SEQ ID NO: 26)
MPKKKRKVGTMTNGKILGLAIGIASVGVGIIEAKTGKVVHANSRLFSAANAENNTE
RRGFRGSRRLNRRKKHRVKRVRDLFEKHEIVTDFRNLNLSPYELRVKGLTEQLTNEE
LFAALRTISKRRGISYLDDAEDDSTGSTDYAKSIDENRRLLKNKTPGQIQLERLEKYG
QLRGNFTVYDENGEAHRLINVESTSDYEKEARKILETQADYNKKITAEFIDDYVEILT
QKRKYYHGPGNEKSRTDYGRFRTDGTTLENIFGILIGKCNFYPDEYRASKASYTAQE
YNFLNDLNNLKVPTETGKLPTEQKESLVEFAKNTATLGPSKLLKEIAKILDCKVDEIK
GYREDDKGKPDLHTFEPYRKLKFNLESINIDDLSREVIDKLADILTLNTEREGIEDAIK
RNLPNQFTEEQISEIIKVRKSQSTAFNKGWHSFSAKLMNELIPELYATSDEQMTILTRL
EKFKVNKKSSKNTKTIDEKEVTDEIYNPVVAKSVRQTIKIINAAVKKYGDFDKIVIEM
PRDKNADDEKKFIDKRNKENKKEKDDALKRAAYLYNSSDKLPDEVFHGNKQLETKI
RLWYQQGERCLYSGKPIPIHDLVHNSNNFEIDHILPLSLSFDDSLANKVLVYDWANQ
EKGQKTPYQVIDSMDAAWSFREMKDYVLKQKGLGKKKRDYLLTTENIDKIEVKKK
FIERNLIDTRYASRVVLNSLQSALRELCKDTKVSVIRGQFTSQLRRKWKIDKSRETYH
HHAVDALIIAASSQLKLWEKQDNLMFIDYGNNQVVDKETGEILSVSDDEYKELVFQP
PYQGFVNTISSKGFEDEILFSYQVDSKYNRKVSDATIYSTRKAKIGKDKKEETYVLGK
IKDIYSQNGFDTFIKKYNKDKTQFLMYQKDPLTWENVIEVILRDYPTTKKSEDGKND
VKCNPFEEYRRENGLICKYSKKGKGTPIKSLKYYDKKLGNCIDITPEESRNKVILQSIN
PWRADVYFNPETLKYELMGLKYSDLSFEKGTGKYHISQEKYDAIKEKEGIGKKSEFK
FTLYRNDLILIKDTASGEQEIYRFLSRTMPNVNHYAELKPYDKEKFDGGQELMEVFG
KVANGGQCLKSLNKSNISIYKVRTDVLGNKYFVKKEGDKPKLNFKNNKKKRPAAT
KKAGQAKKKKGSYPYDVPDYAYPYDVPDYAYPYDVPDYA

In some embodiments, the Lactobacillus kullabergensis strain Biut2 Cas9 has an amino acid sequence at least 80% identical to

(SEQ ID NO: 27)
MPKKKRKVGTMKRVNEDYILGLAIGTNSCGWAVTDKKNNLLKLRGKTAIGSHLFE
EGHTAADRRGFRTTRRRLKRRKWRLRLLEEIFAEPMAKVDPGFFVRLHQSWVSPLD
KDRKKYNAIVFPTAKEDQAFYKHYATIYHLRDELMTQDRQFDLREIFLAIHHIVKYR
GNFLQDTPVKDFEASKIEVGPILSHINNAFAEKIVEDQDPIELNVANAADIEDVIRGKD
AEKTVYKLDKVKKIAKLLTDSTAKEEKNVAKQIANAIMGYKTQFETILDKEIDKSDK
AQWEFKLSDADADDKLDALLPDLDETDQTVVAEIEKLFSAITLSTIVDENKSLSQSM
VEKYKKHKKDYKKLKKYINTLQDQTKAKKLLLAYDLYVNNRHGRLLEAKKTFEDK
KKKKALTKDEFYKIVKDNLDDSDLAHEIQQEIAADNFMPKQRTNSNGVIPFQLHQIE
LDKIIANQGKYYPFLAAENPVEDHRKQAPYKLDELVRFRVPYYVGPMITADEQEKTS
GKSFAWMVRKEDGQITPWNFEQKVDRQESANKFIKRMTIKDTYLLSEDVLPANSLL
YQRFEVLNELNNIRINGSRISVDLKQQIFNDLFEEKKTVTEKSLTSYLKQNLHLPTVEI
KGLADPTKFNSSLASYYHLKSLHVFDKELADPQYQKDFEKIIEYSSIFEDKKIFQDKL
HAEFKWLTPEQFKAISTWRLQGWGRLSRKLLVELHDTNGQNIMEQLWDSQKNFMQI
VTEPDFKDAIAKENQNVTRANGVEEILADAYTSPANKKAIRQVVKVVADVVKAAGG
KKPAQFAIEFTRDPDKNPQLSHIRGTKLLKAYQETAGELVDQKLTDSLKEAMTSRKL
LKDKYFLYFMQAGRDAYTGQKINIDEVSTNYQIDHILPQSFIKDDSFDNRVLTATPLN
AEKSDDVPYKRFANNYVSDMKMTVGEMWKHWQKAGIINKHKLGNLLLDPDRLNK
FQKSGFINRQLVETSQIIKLVSVILQNKYPDAEIITVKAGDNSALRQRLNLYKSRDVND
YHHAIDAYLSIICGNFLYQVYPKYRPYFVYGKYKKFSQDPDLQKEVIKHFKGFTFMW
PLLQKDNSERKAPEKIKENNSDRIVFYKHPDIFDKLRKAYNYKYMLVSRETTTENSG
LFDVTIYPRGERDLAKTRKLIPKSNGLDPKIYGGYSGNTDAYMVIVKIDKGKESIYKV
IGVPMRALASLNRAKKQGNYKEELHQVLEPQIMFDKNGKPKRSVKGFRIIKDHVPFK
QVVLDGDKKFMLNSSTYEINAKQLTLTPETMRIVTDNLKKGEDQDQLLVKAYDEIL
QKVDQYLPLFDVNKFRNSLHLGRAKFLDLAVNDKKITLTNILNGLHDNLVTPDLKNI
GIKTPLGKLQVPSGIVLSSEAILIFQSPTGLFEKRVRIADLKRPAATKKAGQAKKKKG
SYPYDVPDYAYPYDVPDYAYPYDVPDYA

In some embodiments, the Streptococcus suis strain LSS83 Cas9 has an amino acid sequence at least 80% identical to

(SEQ ID NO: 28)
MPKKKRKVGTMSNGKILGLAIGIASVGVGVIDAQTGEIIHASSRIFPSANAANNAERR
TFRGSRRLIRRKKHRIKRLDDLFNDFHINLDGEMSTDNPYVLRVKGLSQKLTVEELYI
SIKNIMKRRGISYLDDAESDNEAGRSDYAKAIERNRQLLTSKTPGEIQLERLEKYGQL
RGNFTIIDEEGQSQQIINVESTSDYVKEVEKILDCQKMYHKFISDEFCDKLIELLREKR
KYYVGPGNEKSRTDYGIYRTDGTTLENLFGILIGKCTFYPDQYRSSRASYTAQEFNFL
NDLNNLTVPTETKKLSQEQKEFLVNYAKETSVLGAGKILQQIAKLADCKVEDIRGYR
LDNKDKPELHTFETYRAMKGLVPLVDIGVLSREQLDILADILTLNTDFEGIREALKKQ
LPNVFDEKQVKGLASFRKSKSQLFAKGWHNLSQKIMLEVIPELYATSDEQMTILTRL
GKFEKSSVAEYPSSINVDEITDEIYNPVVAKSIRQTIKIINASIKKWDEFDQIVIEMPRD
RNEDEEKKRIADGQKANAKEKADSILRAAELYCAGKVLPDYVYNGHNQLATKIRL
WYQQGERCIYTGQPISIHDLIHNQNQYEIDHILPLSLTFDDSLSNKVLVLATANQEKA
QRTPYNYLKSATSAWSYREFKDYVTKRKGIGKKKCEYLTFEEDINGFEVRSKFIQRN
LVDTRYASKVILNALQDYFKISGIQTKVSVVRGQFTSQLRHKWGIEKTRETYHHHAV
DALIIAASSQLRLWKKQESPLVVDYQEGRQVDLETGEILELTDEQYKELVYQPPYQG
FVNTISSSAFDNEILFSYQVDSKVNRKISDATIYATRNAQLGKDKTEGIYVLGKIKDIY
TQAGYEAFLKRYTKDKTSFLMYHKDLDTWEKVIEIILRDYREYDEKGKEIGNPFERY
RRENGYVKKYSRKGNGTAIKSLKYYDNKLGNHIDITPENSRNAVVLQSLKPWRTDV
YFNKETGKYEFLGIKYSDLSFEKGTGEYGISQEKYDSIKIAEGVAKKSIFKFTLYKQDL
LFIKDIENNFGKLLRFTSKNDTSKHYVELKPYDKNKFGTEEPLLPVLGNVAKSGQCIK
GLNKSNISIYKVRTDILGYRHFIKQEGEHPQLKFKKKRPAATKKAGQAKKKKGSYP
YDVPDYAYPYDVPDYAYPYDVPDYA

In some embodiments, the amino acid sequence of the Cas9 protein comprises at least one, at least two, at least three, at least four, at least five, at least six, at least seven, at least eight, at least nine, or at least 10 mutations in SEQ ID NOs: 1, 2, 3, 4, 5, 6, 7, 8, 9, 10, 11, 12, 13 or 14.

In some embodiments, the mutation is an amino acid substitution.

In some embodiments, the Cas9 protein has nickase activity.

In some embodiments, the at least one mutation results in an inactive Cas9 (dCas9).

In some embodiments, the Cas9 protein comprises at least one amino acid mutation in PAM Interacting, HNH and/or RuvC domain.

In some embodiments, the Cas9 protein further comprises a nuclear localization sequence (NLS) and/or a FLAG, HIS or HA tag.

In one aspect, provided herein is an engineered, non-naturally occurring Cas9 fusion protein comprising a Cas9 protein having at least 80% identity to SEQ ID NOs: 1, 2, 3, 4, 5, 6, 7, 8, 9, 10, 11, 12, 13 or 14 and wherein the Cas9 protein is fused to a histone demethylase, a transcriptional activator, or to a deaminase.

In some embodiments, the Cas9 protein is fused to a cytosine deaminase or to an adenosine deaminase.

In some embodiments, the Cas9 protein is fused to an adenosine deaminase and has an amino acid sequence at least 80% identical to

(a)
(SEQ ID NO: 29)
MPAAKRVKLDGSEVEFSHEYWMRHALTLAKRARDEREVPVGAVLVLNNRVIGEGWNRA
IGLHDPTAHAEIMALRQGGLVMQNYRLYDATLYVTFEPCVMCAGAMIHSRIGRVVFGVRN
AKTGAAGSLMDVLHHPGMNHRVEITEGILADECAALLCRFFRMPRRVFNAQKKAQSSTD
GSSGSETPGTSESATPESSGPKKKRKVGTMEKSYSIGLAIGTNSVGWSVITDDYKVPA
KKMRVLGNTDKKYIKKNLLGALLFDSGETAEATRLKRTARRRYTRRKNRLRYLQEI
FAKEMAKVDESFFQRLDESFLTDDDKTFDSHPIFGNKAEEDAYHQKFPTIYHLRKHL
ADSQEKADLRLIYLALAHMIKFRGHFLYDNFNDDNFDWRNIDIQKRYEEFIETYDSTL
GESYLADISVDAASILEEKVSKTERLENLLKYYPTEKKTTFFGNLIKLILGQQAKFKAI
FNLEDEISLQFATPTYDEDLEELLGKIDNGDSYSELFVAAQNLYNTILLASFLKTDNKS
AKAPLSTSMIERYENHKKDLAKLKDFVKKNCPDQYHDIFRDKSKNGYAGYIDNGVS
QNDFYTFIGKCLEESLKKDKGAQYFLDKIDRDDFLRKQRTFDNGAFPYQIHLQEMHA
ILRRQGDYYPFLKENQDKIEKILTFRIPYYVGPLARKDSRFAWANYRSDEAITPWNFD
EVVDKEKSAEKFITRMTLNDLYLPEEKVLPKHSLIYETFTVYNELTNIKYVNDQGNAI
HFDSELKEKIFNQLFKENRKVSKKTLIDFLNNSEGIYTDKLVGIDEEVKYLNASLGTY
HDLKKILESFMDDEINEKIIEDIIQTLTLFEDIEMKRQRLQKYDDIFTPKQLKELARRNY
TGWGRLSYKLINGIRNKENNKTILDYLKNGNRNFMQLINDDRLSFKQIIIDARKIEKL
DNIESVVYNLPGSPAIKKGILQSIKIVDELVKVMGHNPDNIVIEMARENQTTNQGKNR
SQQRLKRLQDSMSNFKDSSISLKDVDNSDLQNDRLFLYYIQNGKDMYTGEELDIDHL
SDYDIDHIIPQSFIKDNSIDNRVLTSSAKNRGKSDDVPGRDVVLKMKPFWKKLYDVK
LISKRKFDNLTKSEHGGLTESDKAGFIKRQLVETRQITKYVAQILDGRFNTKRDDNNK
VIRDVKVITLKSSLVSQFRKDFGFYKVREINDYHHAHDAYLNAVVGTAILKKYPKLA
PEFVYGEYKKCDVRKLIAKSGDKSEIGKATAKYFFYSNLMNFFKRVIRYSNGMIVVR
PVIEYSKDTGEIAWDKEKDFKTVCKVLSCPQVNIVKKVEKQSHGLDRGKPKGFYNA
NPSPKPKKGSKVNLVPIKANLNPKNYGGYAGISNSYAVLVDATIEKGAKKKLTRIQE
FQGISIIDREKYEKNKVEFLKGLGYKEIYSIITLPKYSLFELADGSRRMLASILSTNNKR
GEIHKGNELVLPAKYIPLLYHANRIHNTFETGHREYVEKHIAEFKEIAEIILEFNNKYV
NAKKNSSIIEKALESFDSFSLDEICDSFVGKLKKNNTKKNSGLFELVSLGSASDFEFLE
TKVPRYRDYTPSSLLNATLIHQSITGLYETRIDLSKLGEEKRPAATKKAGQAKKKKG
SYPYDVPDYAYPYDVPDYAYPYDVPDYA.
(b)
(SEQ ID NO: 30)
MPAAKRVKLDGSEVEFSHEYWMRHALTLAKRARDEREVPVGAVLVLNNRVIGEGWNRA
IGLHDPTAHAEIMALRQGGLVMQNYRLYDATLYVTFEPCVMCAGAMIHSRIGRVVFGVRN
AKTGAAGSLMDVLHHPGMNHRVEITEGILADECAALLCRFFRMPRRVFNAQKKAQSSTD
GSSGSETPGTSESATPESSGPKKKRKVGTMTKDYTIGLAIGTNSVGWAVLTDDYQL
MKRKMSVHGNTEKKKIKKNFWGARLFDEGQTAEFRRTKRTNRRRLARRKYRLSKL
QDLFAEELCKQDDCFFVRLEESFLVPEEKQYKPASIFPTLEEEKEYYQKYPTIYHLRQ
KLVDSTEKEDLRLVYLALAHLLKYRGHFLFEGDLDTENTSIEESFRVFLEQYSKQSDQ
PLIVHQPVLTILTDKLSKTKKVEEILKYYPTEKINSFFAQCLKLIVGNQANFKRIFDLEA
EVKLQFSKETYEEDLESLLEKIGDEYLDIFLQAKKVHDAILLSEIISSTVKHTKAKLSS
GMVERYERHKADLAKFKQFVKENVPQKSTAFFKDTTKNGYAGYIKGKTTQEEFYKF
VKKELSGVVGSEPFLEKIDQETFLLKQRTYTNGVIPHQVHLIELKAIIDQQKQHYPFLE
EAGPKIIALFKFRIPYYVGPLAKEQEASSFAWIERKTAEKIHPWNFSEVVDIEKSAMRF
IQRMTKQDTYLPTEKVLPKNSLLYQKYMIFNELTKVSYKDERGVKQYFSGDEKQQIF
KQLFQKERGKITVKKLQNFLYTHYHIENAQIFGIEKAFNASYSTYHDFMKLAKTNQK
AMQEWLEQPEMEPIFEDIVKILTIFEDRQMIKHQLSKYQEVFGEKLLKEFARKHYTG
WGRFSAKLIHGIRDRKTNKTILDYLINDDDVPANRNRNLMQLINDEHLSFKEEIAKAT
VFSKHKSLVDVIQDLPGSPAIKKGIWQSLKIVEELIAIIGYKPKNIVIEMARENQKTHR
TKPRLKALENGLKQIGSTLLKEQPTDNKALQKERLYLYYLQNGRDMYTGEPLEIENL
HQYEVDHIIPRSFIVDNSIDNKVLVARKQNQKKRDDVPKKQIVNEQRIFWNQLKEAK
LISPKKYAYLTKIELTPEDKARFIQRQLVETRQITKHVANILHQSFHQEEEGTDCDGV
QIITLKATLTSQFRQTFGLYKVREINPHHHAHDAYLNGFIANVLLKRYPKLAPEFVYG
KYVKYSLARENKATAKKEFYSNILKFFESDEPFCDENGEIYWEKSHHLPRIKKVLSSH
QVNVVKKVEQQKGGFYKETVNSKEKPDKLIERKNNWDVTKYGGFGSPVIAYAIAFV
YAKGKTQKKTRAIEGITIMEQAAFEKDPTTFLKEKGFPQVTEFIKLPKYTLFEFDNGR
RRFLASHKESQKGNPFILSDQLVTLLYHAQHYDKITYQESFDYVNTHLSDFSAILTEV
LAFAEKYTLADKNIERIQELYEENKYGEISMIAQSFLQLLQFNAIGAPADFKFFGVTIP
RKRYTSLTEIWDATIIYQSVTGLYETRIRMGDLWAGEQKRPAATKKAGQAKKKKG
SYPYDVPDYAYPYDVPDYAYPYDVPDYA
(c)
(SEQ ID NO: 31)
MPAAKRVKLDGSEVEFSHEYWMRHALTLAKRARDEREVPVGAVLVLNNRVIGEGWNRA
IGLHDPTAHAEIMALRQGGLVMQNYRLYDATLYVTFEPCVMCAGAMIHSRIGRVVFGVRN
AKTGAAGSLMDVLHHPGMNHRVEITEGILADECAALLCRFFRMPRRVFNAQKKAQSSTD
GSSGSETPGTSESATPESSGPKKKRKVGTMTNGKILGLAIGVASVGVGIIEAKTGKVI
HANSRLFSAANAENNAERRGFRGARRLTRRKKHRVKRVRDLFEKYDISTDFRNLNL
NPYELRVKGLSEQLTNEELFAALRTIAKRRGISYLDDAEDDSTGSSDYAKSIDENRRL
LKSMTPGQIQLERLEKYGQLRGNFTVYDENGEAHRLINVFSTSDYKNEARKILETQS
NYNKQITDEFIEDYIEILTQKRKYYHGPGNEKSRTDYGRFRTDGTTLENIFGILIGKCSF
YPEEYRASKASYTAQEFNFLNDLNNLKVPTETGKLSTEQKEYLVDFAKNSKTLGASK
LLKEIAKLVDGDVKDISGYREDSKGKPDLHTFEPYRKLKFNLTTVDIDNLSRDILDKL
ANILTLNTEREGIEDAINRNLPEQFTKEQISEIVQIRKSQSSAFNKGWHSFSAKLMNELI
PELYATSEEQMTILTRLEKFKATKKSSKNTKTIDEKEITDEIYNPVVAKSVRQTIKIINA
AVKKYGDFDKIVIEMPRDKNADDEKKFIDKKNKENKKEKDDSLKRAAYLYNGTDK
LPDDVFHGNKQLATKIRLWYQQGERCLYSGKPILIQDLVHNSNNFEIDHILPLSLSFD
DSLANKVLVYAWTNQEKGQKTPYQVIDSMDAAWSFREMKDYVLKQKGIGKKKRE
YLLTTENIDKIEVKKKFIERNLVDTRYASRVVLNSLQTALKELGKDTKVSVVRGQFTS
QLRRKWKIDKSRETYHHHAVDALIIAASSQLKLWQKHENLMFENYGENQVVNKET
GEILSISDDEYKELVFQPPYQGFVNTISSKAFEDEILFSYQVDSKFNRKVSDATIYSTRK
AKLGKDKKEETYVLGKIKDIYSQDGFDTFIKRYKKDKTQFLMYQKDPLTWENVIEVI
LRDYPTTKKSEDGKNDVKCNPFEEYRRENGLICKYSKKGKGTPIKSLKYYDKKLGN
HISITPKESKNDVVLQSLNPWRADLYFNPDTLKYELMGLKYSDLSFEKGTGKYHISQ
EKYDEIKEKEGIGQNSEFKFTLYRNDLILIKDTESGEQEIYRFLSRTMPNVKHYVELKP
YDKEKFNGGQELIKSLGEADKVGRCLKGLSKPGISIYKVRTDVLGNKFFVKKEGDKP
KLDFKNNKKKRPAATKKAGQAKKKKGSYPYDVPDYAYPYDVPDYAYPYDVPDYA.
(d)
(SEQ ID NO: 32)
MPAAKRVKLDGSEVEFSHEYWMRHALTLAKRARDEREVPVGAVLVLNNRVIGEGWNRA
IGLHDPTAHAEIMALRQGGLVMQNYRLYDATLYVTFEPCVMCAGAMIHSRIGRVVFGVRN
AKTGAAGSLMDVLHHPGMNHRVEITEGILADECAALLCRFFRMPRRVFNAQKKAQSSTD
GSSGSETPGTSESATPESSGPKKKRKVGTMNGLVLGLAIGIASVGVGILNKETGEIIH
VNSRIFPAATADSNVERRGFRQGRRLGRRKKHRSARLNDLFEEFGFITDFSAVPLNLN
PYALRVKGLSEELTNEELFIALKNIIKRRGISYLDDASEDGETASNEYGKAVEENRKL
LADKTPGQIQLERFEKYGQVRGDFTVVENGENHRLINVFSTSAYKKEAERILRRQQE
FNVRISDEFIEAYLTILTGKRKYYHGPGNEKSRTDYGRFRTDGTTLDNIFGILIGKCTF
YPDEYRAAKASYTAQEFNLLNDLNNLTVPTETKKLRPEQKRQIVEYARTAKTLGTPT
LLKYIAKLVDGSIDDIKGYRIDKSDKPEMHTFDAYRKMRTLELVDVDILSRETLDDL
AYILTLNTESEGILEALNSKMPGTFTKEQIDELIQFRKKNSAVFGKGWHNFSLKLMNE
LISELYETSEEQMTILTRLGKQRSREISKRTKYIDEKELTDEIYNPVVAKSVRQAIKIIN
LATKKYGIFDNIVIEMARESNEDDEKKAIQNVQKANEDEKKAAMEKAADLYNGKKE
LPDSIFHGHKELATKIRLWHQQGERCLYTGKNISIHDLIHNPHQYEIDHILPLSLSFDD
GLANKVLVLATANQEKGQRTPFQALDSMDDAWSYIEFKQYVRNSKSLSNKKKDYL
LTEEDISKIEVKQKFIERNLVDTRYSSRVVLNTLQEFYKTNDFDTKISVVRGQFTSQLR
RKWKIEKSRDTYHHHAVDALIIAASSQLRLWKKQNNPLISYREGQLVDPETGEILSLT
DDEYKELVFRPPYDYFVDTLKSKSFEDSILFSYQVDSKYNRKISDATIYGTRKAQLGK
DKQEETYVLGKIKDIYSQKGYEDFIKRYKKDTTQFLMYHKDPQTFAKVIEEILKTYPD
KELNEKGKEIPCNPFEKYRQENGPIRKYSKKGKGPEIKSLKYYDNKLGNHIDITPVNS
QNQVVLQSLKPWRTDVYFNPQTSKYELMGLKYSDLRFEKGSGSYGISPEKYNKVKA
KEGVDEDSEFKFTLYKNDLILIKDTETGEQQLFRYGSRNDTSKHYVELKPYEKAKFE
GNQQLMNLLGTVAKGGQCLKGINKPNLSIYKVKTDVLGNKHFIKKEGDQPQLNFKK
KIKRPAATKKAGQAKKKKGSYPYDVPDYAYPYDVPDYAYPYDVPDYA.
(e)
(SEQ ID NO: 33)
MPAAKRVKLDGSEVEFSHEYWMRHALTLAKRARDEREVPVGAVLVLNNRVIGEGWNRA
IGLHDPTAHAEIMALRQGGLVMQNYRLYDATLYVTFEPCVMCAGAMIHSRIGRVVFGVRN
AKTGAAGSLMDVLHHPGMNHRVEITEGILADECAALLCRFFRMPRRVFNAQKKAQSSTD
GSSGSETPGTSESATPESSGPKKKRKVGTMNNSYILGLAIGITSVGYGIIEYETRDVID
AGVRLFKEANVENNEGRRSKRGARRLKRRRRHRLQRVKKMLFDYKLLNEDSEISGI
NPYEARVKGLSEKLSDEEFSAALLHLAKRRGVHNVSDVEEDTGNELSTKEQIARNNK
ALEDKYVAELQLERLKEQGEVRGAANRFKTSDYIKEAKQLLKTQSDYHKIDETFIET
YISLLETRRTYYEGPGEGSPFGWKDIKEWYEMLMGHCTYFPEELRSVKYAYNADLY
NALNDLNNLVIARDENEKLEYYEKFQIIENVFKQKKKPTLKQIAKEILVNEEDIKGYR
VTSTGKPEFTNFKIYHDIKGITSRKEILENADLLNQIAEILTIYQSAEDIQEELAELEPKL
TQEEIEQISNLTGYTGTHRLSLKAINLILDELWNTSDNQMTIFNRLKLVPKKVDLSQQ
KEIPTSLVDDFILSPVVKRSFIQSIKVINAIIKKFGLPKDIIIELAREKNSKEAQKFINEMQ
KRNRQTNERIEKIIKETGKEKAKFLIEKIKLHDMQEGKCLYSLESIPLEDLLNNPYHYE
VDHIIPRSVSFDNSFNNKVLVKQEENSKKGNRTPFQYLSSSDAKISYETFKKHILNLSK
GKGRVSKKKKEYLLEERDINRFSVQKDFINRNLVDTRYATRELMNLLRSYFRVNDL
DVKVKSINGGFTSFLRTKWKFKKERNQGYKHHAEDALVIANADFIFKEWKKLDTTN
KVMENQTVEEQQAENMPGIETDDEYKEIFVIPRQIQSIKDFKDYKYSHRVDKKPNRE
LVNDTLYSTRKDDKGNTLIINNIKGLYDKDNDKLKNLIKKSPEKLLMYHHDPQTYQK
LKTIMEQYSNEKNPLYKYHEETGNYLTKYSKKDNGPIIKKVKYYGKKLNAHLDITN
DYSNSQNKIVKLSLKPYRFDVYLDNGGYKFVTVKNLDVIKKEGFFKIDSNAYEKAKS
EKKIDENAVFIASFYNNDLIKIDGELYRIVGVNNDTRNVVELNMIPITYKEYLENINDK
RTPRILKTISQKTYSIEKYSTDILGNLYKVKSKKKPQMIMKGKRPAATKKAGQAKK
KKGSYPYDVPDYAYPYDVPDYAYPYDVPDYA.
(f)
(SEQ ID NO: 34)
MPAAKRVKLDGSEVEFSHEYWMRHALTLAKRARDEREVPVGAVLVLNNRVIGEGWNRA
IGLHDPTAHAEIMALRQGGLVMQNYRLYDATLYVTFEPCVMCAGAMIHSRIGRVVFGVRN
AKTGAAGSLMDVLHHPGMNHRVEITEGILADECAALLCRFFRMPRRVFNAQKKAQSSTD
GSSGSETPGTSESATPESSGPKKKRKVGTMENKNYSIGLAIGTNSVGWAVITDDYKV
PSKKMKVFGNTDKHFIKKNLIGALLFDEGATAEDRRLKRTARRRYTRRKNRLRYLQ
EIFSEEISKLDSSFFHRLDDSFLVPKDKRGSKYPIFATLEEEKEYHKKFPTIYHLRKHLA
DSKEKTDLRLIYLALAHMIKYRGHFLYEESFDIKNNDIQKIFNEFISIYDNTFEGSSLSG
QNAQVEAIFTDKISKSAKRERVLKLFSDEKSTSLFSEFLKLIVGNQADFKKHFDLEEK
APLQFSKDTYDEDLENLLGQIGDGFTDLFLVAKKLYDAILLSGILTVTDPSTKAPLSAS
MIERYESHQKDLAALKQFIKNNLPKRYNEVFSDQSKDGYAGYIDGKTTQEAFYKYIK
NLLSKFEGADYFLDKIEREDFLRKQRTFDNGSIPHQIHLQEMNAILRRQGEHYPFLKE
NREKIEKILTFRIPYYVGPLARGNRDFAWLTRNSDQAIRPWNFEEIVDKASSAEEFINK
MTNYDLYLPEEKVLPKHSLLYETFAVYNELTKVKFIAEGLRDYQFLDSGQKKQIVNQ
LFKEKRKVTEKDIIHYLHNVDGYDGIELKGIEKQFNASLSTYHDLLKIIKDKEFMDDP
KNEEILENIVHTLTIFEDREMIKQRLAQYDSIFDEKVIKALTRRHYTGWGKLSAKLING
ICDKQTGDTILDYLIDDGKINRNFMQLINDDGLSFKEIIQKAQVVGKTDDVKQVVQEL
PGSPAIKKGILQSIKIVDELVKVMGYAPESIVIEMARENQTTARGKKNSQQRYKRIED
SLKNLAPGLDSNILKENPTDNIQLQNDRLFLYYLQNGKDMYTGKPLDIDQLSSYDID
HIIPQAFIKDDSIDNRVLTSSKDNRGKSDNVPSLEVVQKRKAFWQQLLDSKLISERKF
NNLTKAERGGLDERDKVGFIRRQLVETRQITKHVAQILDASFNTEVNEKNQKIRTVKI
ITLKSNLVSNFRKEFELYKVREINDYHHAHDAYLNAVVAKAILKKYPKLEPEFVYGD
YQKYDLKRYISRFKPSKEIEKATEKYFFYSNLLNFFKEEVHYADGIIVKRENIEYSKDT
GEIAWNKEKDFATIKKVLSYPQVNIVKKTEIQTHGLDRGKPKGLFNSNPSPKPSEDSK
ENLVPIKQGLDPRKYGGYAGISNSYAVLVKAIVEKGAKKQQKTILEFQGISILDKINFE
NNKENYLLKKRYIEILSTITLPKYSLFEFPDGTRRRLASILSTNNKRGEIHKGNELVLPG
KYTTLLYHAKNINKKLEPEHLEYVEKHRNDFAKLLECVLNFNDKYVGALKNGERIR
QAFTDWETVDIEKLCFSFIGPENSKNAGLFELTSQGSASDFEFLGVKIPRYRDYAPSSL
LKATLIHQSITGLYETRIDLSKLGEDKRPAATKKAGQAKKKKGSYPYDVPDYAYPYD
VPDYAYPYDVPDYA.
(g)
(SEQ ID NO: 35)
MPAAKRVKLDGSEVEFSHEYWMRHALTLAKRARDEREVPVGAVLVLNNRVIGEGWNRA
IGLHDPTAHAEIMALRQGGLVMQNYRLYDATLYVTFEPCVMCAGAMIHSRIGRVVFGVRN
AKTGAAGSLMDVLHHPGMNHRVEITEGILADECAALLCRFFRMPRRVFNAQKKAQSSTD
GSSGSETPGTSESATPESSGPKKKRKVGTMSDLVLGLAIGIGSVGVGILNKVTGEIIHK
NSRIFPAAQAENNLERRTNRQGRRLTRRKKHRRVRLNHLFEESGLITDFTKVSINLNP
YQLRVKGLTAELSNEELFIALKNMVKHRGISYLDDASDDGNSSVGDYAQIVKENSK
QLETKTPGQIQLERYQKYGQLRGDFTVEEDGRKHRLINVFPTSAYHAEALRILQTQQ
EFNPQITDEFINSYLEILTGKRKYYHGPGNEKSRTDYGKYTTKKDAQGQYITLNNIFGI
LIGKCTFYPEEYRAAKASYTAQEFNLLNDLNNLTVPTETKKLSEEQKYQIITYVKNEK
AMGPAKLFKYIAKLLSCDVADIKGYRIDKSDKAEIHTFEAYRKMKTLETLDVKKMA
REELDKLAYVLTLNTEREGIQEALDHEFADGTFSQEQVDELVQFRKANSSIFGKGWH
SFSVKLMMELIPELYATSEEQMTILTRLGKQKTTSSSNKTKYIDEKQLTEEIYNPVVA
KSVRQAIKIVNAAIKKYGDFDNIVIEMARETNEDDEKKAIQKIQKANKAEKDAAMRK
AANQYNGKAELPHSVFHGHKQLATKIRLWHQQGERCLYTGKTISIHDLINNPNQFEI
DHILPLSITFDDSLANKVLVYATANQEKGQRTPYQALDSMDDAWSFRELKAFVRESK
ALSNKKKEYLLTEEDISKFDVRKKFIERNLVDTRYASRVVLNALQEHFRTHKIDTKVS
VVRGQFTSQLRRHWGIEKTRDTYHHHAVDALIIAASSQLNLWKKQKNTLVSYSEDQ
LLDIETGELISDDEYKESVFKAPYQHFVDTLKSKEFEDSILFSYQVDSKFNRKISDATIY
ATRKAKLDKEKKEYTYTLGKIKDIYALGTKTPSKTGFYKFLDLYKKDKSQFLMYQK
DRRTWDEVIEKILEQYRPFKEKDKNGKEVDENPFEKYRIENGPIRKYSRKGNGPEIKS
LKYYDNLLGRFVDITPSESKNPVALLSLNPWRTDVYYNTETRKYEFLGLKYADLCFE
KGGSYGISKVKYNKIREKEGIGKNSEFKFTLYKNDLILIKDTETNRQQIFRFWSRTGK
DNPKSFEKHKLELKPYEKTRFEKGEELKVLGKVPPSSNRLQKNMQIENLSIYKVRTD
VLGNQHIIKNEGDKPKLDFKRPAATKKAGQAKKKKGSYPYDVPDYAYPYDVPDYAY
PYDVPDYA.
(h)
(SEQ ID NO: 36)
MPAAKRVKLDGSEVEFSHEYWMRHALTLAKRARDEREVPVGAVLVLNNRVIGEGWNRA
IGLHDPTAHAEIMALRQGGLVMQNYRLYDATLYVTFEPCVMCAGAMIHSRIGRVVFGVRN
AKTGAAGSLMDVLHHPGMNHRVEITEGILADECAALLCRFFRMPRRVFNAQKKAQSSTD
GSSGSETPGTSESATPESSGPKKKRKVGTMNGLVLGLAIGIASVGVGILEKNSGKIIH
ANSRIFPAATADNNVERRKNRQARRLHRRKKHRGVRLQDIFEDYGLLTDFSKVSINL
NPYRLRVDGLDQQLTNEELFIALKNIVKRRGISYLDDASEDGGTVSSDYGKAVEENR
KLLAEKTPGQIQLERFEKYGQVRGDFTVVENGEKRRLINVFSTSAYRKEAERILRKQ
QEFNSKITDEFIEDCLKILTGKRKYYHGPGNEKSRTDYGRFRTDGTTLDNIFGILIGKC
TFYPNEYRASKASHTAQEFNLLNDLNNLTVPTETKKLSEEQKKVIVEYAKEAKTLGA
STLLKYIAKMIDASVDQISGYRVDVNNKPEMHTFEVYRKMQSLETISVGELSRNVLD
ELAHILTLNTEREGIEEAINTKLKDSFSQDQVLELVQFRKNNSSLFSRGWHNFSLKLM
MELIPELYETSEEQMTILTRLGKQKSKETSKRTKYIDEKELTEEIYNPVVAKSVRQAIK
IINEATKKYGIFDNIVIEMARENNEEDAKKDYIKRQKANQDEKNAAMEKAAFQYNG
KKELPDNIFHGHKELATKIRLWHQQGEKCLYTGKNIPISDLIHNQYKYEIDHILPLSLS
FDDSLSNKVLVLATANQEKGQRTPFQALDSMDDGWSYREFKSYVKESKLLGNKKK
EYLLTEEDISKIEVKQKFIERNLVDTRYSSRVVLNALQDFYKAHKFDTTISVVRGQFT
SQLRRKWGLEKSRETYHHHAVDALIIAASSQLRLWKKQNNPLIAYKEGQFVDSETGE
ILSLSDDEYKELVFKAPYDHFVDTLRSKTFEDSILFSYQVDSKYNRKISDATIYATRKA
KLDKDKSEETYVLGKIKDIYSQAGYDAFIKIYNKDKSKFLMYHKDPQTFEKVIEEILR
TYPSKELNDKNKEIPCNPFEKYRQENGPIRKYSKKGNGPEIKCLKYYDNKLGNYIDIT
PDGSDNQVVLQSIAPWRTDVYYNHKTGKYEFLGLKYSDLYFEKGTGKYKISKEKYD
NIKKIEGVVETSEFKFTLYKNDLILIKDVEKGQEQLFRFLSRNNKGKHQVQLKPMNKS
DFEKGEKLIDIFGTVPNSTTQCVKGLNKSNISIFKVKTDVLGKKHIIKKEGDEPKLKFK
RPAATKKAGQAKKKKGSYPYDVPDYAYPYDVPDYAYPYDVPDYA.
(i)
(SEQ ID NO: 37)
MPAAKRVKLDGSEVEFSHEYWMRHALTLAKRARDEREVPVGAVLVLNNRVIGEGWNRA
IGLHDPTAHAEIMALRQGGLVMQNYRLYDATLYVTFEPCVMCAGAMIHSRIGRVVFGVRN
AKTGAAGSLMDVLHHPGMNHRVEITEGILADECAALLCRFFRMPRRVFNAQKKAQSSTD
GSSGSETPGTSESATPESSGPKKKRKVGTMNGLVLGLAIGIASVGVGILEKNSGKIIH
ANSRIFPAATADNNVERRKNRQARRLHRRKKHRGVRLQDIFEDYGLLTDFSKVSINL
NPYRLRVDGLDQQLTNEELFIALKNIVKRRGISYLDDASEDGGTVSSDYGKAVEENR
KLLAEKTPGQIQLERFEKYGQVRGDFTVVENGEKRRLINVFSTSAYRKEAERILRKQ
QEFNSKITDEFIEDCLKILTGKRKYYHGPGNEKSRTDYGRFRTDGTTLDNIFGILIGKC
TFYPNEYRASKASHTAQEFNLLNDLNNLTVPTETKKLSEEQKKVIVEYAKEAKTLGA
STLLKYIAKMIDASVDQISGYRVDVNNKPEMHTFEVYRKMQSLETISVGELSRNVLD
ELAHILTLNTEREGIEEAINTKLKDSFSQDQVLELVQFRKNNSSLFSRGWHNFSLKLM
MELIPELYETSEEQMTILTRLGKQKSKETSKRTKYIDEKELTEEIYNPVVAKSVRQAIK
IINEATKKYGIFDNIVIEMARENNEEDAKKDYIKRQKANQDEKNAAMEKAAFQYNG
KKELPDNIFHGHKELATKIRLWHQQGEKCLYTGKNIPISDLIHNQYKYEIDHILPLSLS
FDDSLSNKVLVLATANQEKGQRTPFQALDSMDDGWSYREFKSYVKESKLLGNKKK
EYLLTEEDISKIEVKQKFIERNLVDTRYSSRVVLNALQDFYKAHKFDTTISVVRGQFT
SQLRRKWGLEKSRETYHHHAVDALIIAASSQLRLWKKQNNPLIAYKEGQFVDSETGE
ILSLSDDEYKELVFKAPYDHFVDTLRSKTFEDSILFSYQVDSKYNRKISDATIYATRKA
KLDKDKSEETYVLGKIKDIYSQAGYDAFIKIYNKDKSKFLMYHKDPQTFEKVIEEILR
TYPSKELNDKNKEIPCNPFEKYRQENGPIRKYSKKGNGPEIKCLKYYDNKLGNYIDIT
PDGSDNQVVLQSIAPWRTDVYYNHKTGKYEFLGLKYSDLYFEKGTGKYKISKEKYD
NIKKIEGVVETSEFKFTLYKNDLILIKDVEKGQEQLFRFLSRNNKGKHQVQLKPMNKS
DFEKGEKLIDIFGTVPNSTTQCVKGLNKSNISIFKVKTDVLGKKHIIKKEGDEPKLKFK
RPAATKKAGQAKKKKGSYPYDVPDYAYPYDVPDYAYPYDVPDYA.
(j)
(SEQ ID NO: 38)
MPAAKRVKLDGSEVEFSHEYWMRHALTLAKRARDEREVPVGAVLVLNNRVIGEGWNRA
IGLHDPTAHAEIMALRQGGLVMQNYRLYDATLYVTFEPCVMCAGAMIHSRIGRVVFGVRN
AKTGAAGSLMDVLHHPGMNHRVEITEGILADECAALLCRFFRMPRRVFNAQKKAQSSTD
GSSGSETPGTSESATPESSGPKKKRKVGTMKEKYILGLALGITSVGYGIINFETKKIID
AGVRLFPEANVDNNEGRRSKRGSRRLKRRRIHRLDRVKSLLTEYNLINREQIPTSNNP
YQIRVKGLSEILSKDELAIALLHLAKRRGIHNINVSSEDEDASNELSTKEQINRNNKLL
KNKYVCEVQLQRLKEGQIRGEKNRFKTTDILKEIDQLLKVQKDYHNLDIDFINQYKEI
VETRREYFEGPGQGSPFGWNGDLKKWYEMLMGHCTYFPQELRSVKYAYSADLFNA
LNDLNNLIIQRNDSEKLEYHEKYHIIENVFKQKKKPTLKQIAKEIGVNPEDIKGYRITK
SGTPQFTEFKLYHDLKSIVFDKSILENEAILDQIAEILTIYQDEESIKEELNKLPEILNEQ
DKAEIAKLTGYNVTHRLSLKCIHLINEELWQTSRNQMEIFNYLNIKPNKVDLSEQNKI
PKDLVDEFILSPVVKRTFIQSINVINKVIEKYGIPEDIIIELARENNSDDRKKFINNLQKK
NEATRKRINEIIGQTGNQNGKRIVEKIRLHDQQEGKCLYSLESIPLMDLLNNPQNYEV
DHIIPRSVAFDNSIHNKVLVKQIENSKKGNRTPYQYLNSSDANLSYNQFKQHILNLSK
SKDRISKKKKDYLLEERDINKFEVQKEFINRNLVDTRYATRELTSYLKAYFSANNMD
VKVKTINGSFTNHLRKVWRFDKYRNHSYKHHAEDALIIANADFLFKENKKLQNANK
ILEKPTIENDTQKVTVEKEEDYNNMFETPKLVEDIKQYRDYKFSHRVDKKPNRQLIK
DTLYSTRMKDEHNYIVQTITDIYGKDNTNLKKQFNKNPEKFLMYQNDPKTFEKLSII
MKQYSDEKNPLAKYYEETGEYLTKYSKKNNGPIVKKIKLLGNKVGNHLDVTNKYE
NSTKKLVKLSIKNYRFDVYLTEKGYKFVTIAYLNVFKKDNYYYIPKDLYQELKAKK
KIKDTDQFIASFYKNDLIKLNGDLYKIIGVNSDDRNIIELDYYDIKYKDYCEINNIKGEP
RIKKTIGKKTESIEKLTTDVLGNLYLHTTEKAPQLIFKRGLKRPAATKKAGQAKKK
KGSYPYDVPDYAYPYDVPDYAYPYDVPDYA.
(k)
(SEQ ID NO: 39)
MPAAKRVKLDGSEVEFSHEYWMRHALTLAKRARDEREVPVGAVLVLNNRVIGEGWNRA
IGLHDPTAHAEIMALRQGGLVMQNYRLYDATLYVTFEPCVMCAGAMIHSRIGRVVFGVRN
AKTGAAGSLMDVLHHPGMNHRVEITEGILADECAALLCRFFRMPRRVFNAQKKAQSSTD
GSSGSETPGTSESATPESSGPKKKRKVGTMKRNYILGLAIGITSVGYGIIDYETRDVID
AGVRLFKEANVENNEGRRSKRGARRLKRRRRHRLQRVKKLLFDYNLLTDHSELSGI
NPYEARVKGLSQKLSEVEFSAALLHLAKRRGVHNVNEVEEDTGNELSTKEQISRNSK
ALEEKYVAELQLERLKTDGEVRGPNNRFKTSDYVKEAKQLLKVQKAYHOLDQSFID
TYIDLLETRRTYYEGPGEGSPFGWKDIKEWYEMLMGHCTYFPEELRSVKYAYNADL
YNALNDLNNLVIARDENEKLEYYEKFQIIENVFKQKKKPTLKQIAKELLVNEEDIKGY
RVTSIGKPEFTNFKIYHDIKGITERKEVLENAELLDQIAEILTIYQSSEDVQEELANLNS
ELTQEEIEQISNLKGYTGTHNLSLKAINLILDELWHTNDNQMAIFNRLKLVPKKVDLS
QQKEIPTTLVDDFILSPVVKRSFIQSIKVINAIIKKYGLPNDIIIELAREKNSKDAQKMIN
EMQKRNRQMNERIEEIIRTTGKENAKYLIEKIKLHDMQEGKCLYSLESIPLEDLLNNP
FNYEVDHIIPRSVSFDNSFNNKVLVKQEENSKKGNRTPFQYLSSSDSKISYETFKKHIL
NLAKGKGRISKTKKEYLLEERDINRFSVQKDFINRNLVDTRYATRELMNLLRSYFRV
NNLDVKVKSINGGFTSFLRRKWKFKKERNKGYKHHAEDALIIANADFIFKEWKKLD
KAKKVMENQTVEEKQAESMPEIETEQEYKEIFITPHQIQHIKGFKDYKYSHRVDKKP
NRELINDTLYSTRKDDKGNTLIVNNLNGLYDKDNDKLKKLMNKSPEKLLMYHHDP
QTYQKLKLIMEQYGDEKNPLYKYYEETGNYLTKYSKKDNGPVIKKIKYYGKNLKAH
LDITDDYPNSRNKVVKLSVKPYRFDVYLDNDIYKFVTVKNLDVIKKEDYYEVNSKC
YKEAKKLKKISDQAEFIASFYNNDLIKINGELYRVIGVNNDLLNRIEVNMINITYREYL
ENMNDKRPPRIIKTIASKTQSIKKYSTDILGNLYEVKSKQKPQMIMKGKRPAATKKA
GQAKKKKGSYPYDVPDYAYPYDVPDYAYPYDVPDYA.
(l)
(SEQ ID NO: 40)
MPAAKRVKLDGSEVEFSHEYWMRHALTLAKRARDEREVPVGAVLVLNNRVIGEGWNRA
IGLHDPTAHAEIMALRQGGLVMQNYRLYDATLYVTFEPCVMCAGAMIHSRIGRVVFGVRN
AKTGAAGSLMDVLHHPGMNHRVEITEGILADECAALLCRFFRMPRRVFNAQKKAQSSTD
GSSGSETPGTSESATPESSGPKKKRKVGTMTNGKILGLAIGIASVGVGIIEAKTGKVV
HANSRLFSAANAENNTERRGFRGSRRLNRRKKHRVKRVRDLFEKHEIVTDFRNLNLS
PYELRVKGLTEQLTNEELFAALRTISKRRGISYLDDAEDDSTGSTDYAKSIDENRRLL
KNKTPGQIQLERLEKYGQLRGNFTVYDENGEAHRLINVFSTSDYEKEARKILETQAD
YNKKITAEFIDDYVEILTQKRKYYHGPGNEKSRTDYGRFRTDGTTLENIFGILIGKCNF
YPDEYRASKASYTAQEYNFLNDLNNLKVPTETGKLPTEQKESLVEFAKNTATLGPSK
LLKEIAKILDCKVDEIKGYREDDKGKPDLHTFEPYRKLKFNLESINIDDLSREVIDKLA
DILTLNTEREGIEDAIKRNLPNQFTEEQISEIIKVRKSQSTAFNKGWHSFSAKLMNELIP
ELYATSDEQMTILTRLEKFKVNKKSSKNTKTIDEKEVTDEIYNPVVAKSVRQTIKIINA
AVKKYGDFDKIVIEMPRDKNADDEKKFIDKRNKENKKEKDDALKRAAYLYNSSDK
LPDEVFHGNKQLETKIRLWYQQGERCLYSGKPIPIHDLVHNSNNFEIDHILPLSLSFDD
SLANKVLVYDWANQEKGQKTPYQVIDSMDAAWSFREMKDYVLKQKGLGKKKRD
YLLTTENIDKIEVKKKFIERNLIDTRYASRVVLNSLQSALRELCKDTKVSVIRGQFTSQ
LRRKWKIDKSRETYHHHAVDALIIAASSQLKLWEKQDNLMFIDYGNNQVVDKETGE
ILSVSDDEYKELVFQPPYQGFVNTISSKGFEDEILFSYQVDSKYNRKVSDATIYSTRKA
KIGKDKKEETYVLGKIKDIYSQNGFDTFIKKYNKDKTQFLMYQKDPLTWENVIEVIL
RDYPTTKKSEDGKNDVKCNPFEEYRRENGLICKYSKKGKGTPIKSLKYYDKKLGNCI
DITPEESRNKVILQSINPWRADVYFNPETLKYELMGLKYSDLSFEKGTGKYHISQEKY
DAIKEKEGIGKKSEFKFTLYRNDLILIKDTASGEQEIYRFLSRTMPNVNHYAELKPYD
KEKFDGGQELMEVFGKVANGGQCLKSLNKSNISIYKVRTDVLGNKYFVKKEGDKPK
LNFKNNKKKRPAATKKAGQAKKKKGSYPYDVPDYAYPYDVPDYAYPYDVPDYA.
(m)
(SEQ ID NO: 41)
MPAAKRVKLDGSEVEFSHEYWMRHALTLAKRARDEREVPVGAVLVLNNRVIGEGWNRA
IGLHDPTAHAEIMALRQGGLVMQNYRLYDATLYVTFEPCVMCAGAMIHSRIGRVVFGVRN
AKTGAAGSLMDVLHHPGMNHRVEITEGILADECAALLCRFFRMPRRVFNAQKKAQSSTD
GSSGSETPGTSESATPESSGPKKKRKVGTMKRVNEDYILGLAIGTNSCGWAVTDKK
NNLLKLRGKTAIGSHLFEEGHTAADRRGFRTTRRRLKRRKWRLRLLEEIFAEPMAKV
DPGFFVRLHQSWVSPLDKDRKKYNAIVFPTAKEDQAFYKHYATIYHLRDELMTQDR
QFDLREIFLAIHHIVKYRGNFLQDTPVKDFEASKIEVGPILSHINNAFAEKIVEDQDPIE
LNVANAADIEDVIRGKDAEKTVYKLDKVKKIAKLLTDSTAKEEKNVAKQIANAIMG
YKTQFETILDKEIDKSDKAQWEFKLSDADADDKLDALLPDLDETDQTVVAEIEKLFS
AITLSTIVDENKSLSQSMVEKYKKHKKDYKKLKKYINTLQDQTKAKKLLLAYDLYV
NNRHGRLLEAKKTFEDKKKKKALTKDEFYKIVKDNLDDSDLAHEIQQEIAADNFMP
KQRTNSNGVIPFQLHQIELDKIIANQGKYYPFLAAENPVEDHRKQAPYKLDELVRFR
VPYYVGPMITADEQEKTSGKSFAWMVRKEDGQITPWNFEQKVDRQESANKFIKRMT
IKDTYLLSEDVLPANSLLYQRFEVLNELNNIRINGSRISVDLKQQIFNDLFEEKKTVTE
KSLTSYLKQNLHLPTVEIKGLADPTKFNSSLASYYHLKSLHVFDKELADPQYQKDFE
KIIEYSSIFEDKKIFQDKLHAEFKWLTPEQFKAISTWRLQGWGRLSRKLLVELHDTNG
QNIMEQLWDSQKNFMQIVTEPDFKDAIAKENQNVTRANGVEEILADAYTSPANKKAI
RQVVKVVADVVKAAGGKKPAQFAIEFTRDPDKNPQLSHIRGTKLLKAYQETAGELV
DQKLTDSLKEAMTSRKLLKDKYFLYFMQAGRDAYTGQKINIDEVSTNYQIDHILPQS
FIKDDSFDNRVLTATPLNAEKSDDVPYKRFANNYVSDMKMTVGEMWKHWQKAGII
NKHKLGNLLLDPDRLNKFQKSGFINRQLVETSQIIKLVSVILQNKYPDAEIITVKAGD
NSALRQRLNLYKSRDVNDYHHAIDAYLSIICGNFLYQVYPKYRPYFVYGKYKKFSQ
DPDLQKEVIKHFKGFTFMWPLLQKDNSERKAPEKIKENNSDRIVFYKHPDIFDKLRK
AYNYKYMLVSRETTTENSGLFDVTIYPRGERDLAKTRKLIPKSNGLDPKIYGGYSGN
TDAYMVIVKIDKGKESIYKVIGVPMRALASLNRAKKQGNYKEELHQVLEPQIMFDK
NGKPKRSVKGFRIIKDHVPFKQVVLDGDKKFMLNSSTYEINAKQLTLTPETMRIVTD
NLKKGEDQDQLLVKAYDEILQKVDQYLPLFDVNKFRNSLHLGRAKFLDLAVNDKKI
TLTNILNGLHDNLVTPDLKNIGIKTPLGKLQVPSGIVLSSEAILIFQSPTGLFEKRVRIA
DLKRPAATKKAGQAKKKKGSYPYDVPDYAYPYDVPDYAYPYDVPDYA.
(n)
(SEQ ID NO: 42)
MPAAKRVKLDGSEVEFSHEYWMRHALTLAKRARDEREVPVGAVLVLNNRVIGEGWNRA
IGLHDPTAHAEIMALRQGGLVMQNYRLYDATLYVTFEPCVMCAGAMIHSRIGRVVFGVRN
AKTGAAGSLMDVLHHPGMNHRVEITEGILADECAALLCRFFRMPRRVFNAQKKAQSSTD
GSSGSETPGTSESATPESSGPKKKRKVGTMSNGKILGLAIGIASVGVGVIDAQTGEIIH
ASSRIFPSANAANNAERRTFRGSRRLIRRKKHRIKRLDDLFNDFHINLDGEMSTDNPY
VLRVKGLSQKLTVEELYISIKNIMKRRGISYLDDAESDNEAGRSDYAKAIERNRQLLT
SKTPGEIQLERLEKYGQLRGNFTIIDEEGQSQQIINVESTSDYVKEVEKILDCQKMYHK
FISDEFCDKLIELLREKRKYYVGPGNEKSRTDYGIYRTDGTTLENLFGILIGKCTFYPD
QYRSSRASYTAQEFNFLNDLNNLTVPTETKKLSQEQKEFLVNYAKETSVLGAGKILQ
QIAKLADCKVEDIRGYRLDNKDKPELHTFETYRAMKGLVPLVDIGVLSREQLDILADI
LTLNTDFEGIREALKKQLPNVFDEKQVKGLASFRKSKSQLFAKGWHNLSQKIMLEVI
PELYATSDEQMTILTRLGKFEKSSVAEYPSSINVDEITDEIYNPVVAKSIRQTIKIINASI
KKWDEFDQIVIEMPRDRNEDEEKKRIADGQKANAKEKADSILRAAELYCAGKVLPD
YVYNGHNQLATKIRLWYQQGERCIYTGQPISIHDLIHNQNQYEIDHILPLSLTFDDSLS
NKVLVLATANQEKAQRTPYNYLKSATSAWSYREFKDYVTKRKGIGKKKCEYLTFEE
DINGFEVRSKFIQRNLVDTRYASKVILNALQDYFKISGIQTKVSVVRGQFTSQLRHKW
GIEKTRETYHHHAVDALIIAASSQLRLWKKQESPLVVDYQEGRQVDLETGEILELTDE
QYKELVYQPPYQGFVNTISSSAFDNEILFSYQVDSKVNRKISDATIYATRNAQLGKDK
TEGIYVLGKIKDIYTQAGYEAFLKRYTKDKTSFLMYHKDLDTWEKVIEIILRDYREYD
EKGKEIGNPFERYRRENGYVKKYSRKGNGTAIKSLKYYDNKLGNHIDITPENSRNAV
VLQSLKPWRTDVYFNKETGKYEFLGIKYSDLSFEKGTGEYGISQEKYDSIKIAEGVAK
KSIFKFTLYKQDLLFIKDIENNFGKLLRFTSKNDTSKHYVELKPYDKNKFGTEEPLLPV
LGNVAKSGQCIKGLNKSNISIYKVRTDILGYRHFIKQEGEHPQLKFKKKRPAATKKA
GQAKKKKGSYPYDVPDYAYPYDVPDYAYPYDVPDYA.

In some embodiments, the Cas9 protein is fused to a cytosine deaminase.

In some embodiments, the Streptococcus equinus ATCC 33317 Cas9 protein recognizes a PAM consensus sequence comprising 5′-NRGNR-3′.

In some embodiments, the Enterococcus hirae strain F1129E recognizes a PAM consensus sequence comprising 5′-NRG-3′

In some embodiments, the Streptococcus equinus strain AG46 recognizes a PAM consensus sequence comprising 5′-NNGR-3′.

In some embodiments, the Staphylococcus warneri strain 691 recognizes a PAM consensus sequence comprising 5′-NNGR-3′.

In some embodiments, the Staphylococcus sciuri strain SNUC 2430 recognizes a PAM consensus sequence comprising 5′-NNGR-3′.

In some embodiments, the Staphylococcus simulans strain 19 recognizes a PAM consensus sequence comprising 5′-NNGRRT-3′.

In some embodiments, the Streptococcus intermedius B196 strain G1552 recognizes a consensus PAM sequence comprising 5′-NNAAAA-3′.

In some embodiments, the Streptococcus sanguinis SK330 recognizes a consensus PAM sequence comprising 5′-NGGNG-3′.

In some embodiments, the Streptococcus sp. C150 recognizes a consensus PAM sequence comprising 5′-NNGNRG-3′.

In some embodiments, the Streptococcus oralis subsp. oralis strain RH_1735_08 recognizes a consensus PAM sequence comprising 5′-NNAAAC-3′.

In some embodiments, the Streptococcus oralis SK313 recognizes a consensus PAM sequence comprising 5′-NNRAAG-3′.

In some embodiments, the Streptococcus gallolyticus strain AM24-4 recognizes a consensus PAM sequence comprising 5′-NNAYAA-3′.

In some embodiments, the Lactobacillus kullabergensis strain Biut2 recognizes a consensus PAM sequence comprising 5′-NNGAAA-3′.

In some embodiments, the Streptococcus suis strain recognizes a consensus PAM sequence comprising 5′-NNAAA-3′ (H=A, C or T; R=A or G).

In some embodiments, a nucleic acid encoding the Cas9 protein is provided.

In some embodiments, the nucleic acid is codon-optimized for expression in mammalian cells.

In some embodiments, the nucleic acid is codon-optimized for expression in human cells.

In some embodiments, a eukaryotic cell comprising the Cas9 protein is provided.

In some embodiments, the cell is a human cell. In some embodiments, the cell is a plant cell.

In one aspect, a method of cleaving a target nucleic acid in a eukaryotic cell is provided comprising: contacting the cell with a Cas9 as described herein, and an RNA guide or a nucleic acid encoding the RNA guide, wherein the RNA guide comprises a direct repeat sequence and a spacer sequence capable of hybridizing to the target nucleic acid, and wherein the Cas9 protein is capable of binding to the RNA guide and of causing a break in the target nucleic acid sequence complementary to the RNA guide.

In one aspect, a method of altering expression of a target nucleic acid in a eukaryotic cell is provided comprising: contacting the cell with a Cas9 as described herein, and an RNA guide or a nucleic acid encoding the RNA guide, wherein the RNA guide comprises a direct repeat sequence and a spacer sequence capable of hybridizing to the target nucleic acid, and wherein the Cas9 protein is capable of binding to the RNA guide and of causing a break in the target nucleic acid sequence complementary to the RNA guide.

In one aspect, a method of altering expression of a target nucleic acid in a eukaryotic cell is provided comprising: contacting the cell with a Cas9 as described herein, and an RNA guide or a nucleic acid encoding the RNA guide, wherein the RNA guide comprises a direct repeat sequence and a spacer sequence capable of hybridizing to the target nucleic acid, and wherein the Cas9 protein is capable of binding to the RNA guide and editing the target nucleic acid sequence complementary to the RNA guide.

In one aspect, a method of modifying a target nucleic acid in a eukaryotic cell is provided comprising: contacting the cell with a Cas9 as described herein, and an RNA guide or a nucleic acid encoding the RNA guide, wherein the RNA guide comprises a direct repeat sequence and a spacer sequence capable of hybridizing to the target nucleic acid, and wherein the Cas9 protein is capable of binding to the RNA guide and editing the target nucleic acid sequence complementary to the RNA guide.

In some embodiments, the Cas9 protein is an inactive Cas9 (dCas9).

In some embodiments, the dCas9 is fused to a deaminase.

In some embodiments, the RNA guide comprises a crRNA and a tracrRNA.

In some embodiments, the RNA guide comprises a sgRNA.

In some embodiments, the sgRNA for use with Seq2Cas9 comprises a scaffold comprising a sequence having at least 80% identity to 5′-GUUUUAGUACUCUGUAAUUUUGAAAAAAAUUACAGAAUCUACUAAAACAAGGC AAAAUGCCGUGUUUAUCUCGUCAACUUGUUGACGAGAUUUUUUU-3′ (SEQ ID NO: 43).

In some embodiments, the sgRNA for use with EhiCas9 comprises a scaffold comprising a sequence having at least 80% identity to 5′-GUUUUAGAGCUAUGUUGGAAACAACAUAGCGAGUUAAAAUAAGGCAUUGUCCG UUAUCAGCUUUUAAAGCAAGCACUGUCUCGGUGCUUUUUU-3′ (SEQ ID NO: 44).

In some embodiments, the sgRNA for use with SeqCas9 comprises a scaffold comprising a sequence having at least 80% identity to 5′-GUUUUUGUACUCUCAAGAUUUGAAAAAAUCUUGCAGAGCCUACAAAGAUAAGG CUUCAUGCCGAAUUCAAGCACCCCAUGUUUACAUGGGGUGCUUUU-3′ (SEQ ID NO: 45).

In some embodiments, the sgRNA for use with SsiCas9 comprises a scaffold comprising a sequence having at least 80% identity to 5′-GUUUUAGUACUCUGUAAUUUUGAAAAAAAUUACAGAAUCUACUAAAACAAGGC AAAAUGCCGUGUUUAUCUCGUCAACUUGUUUGGCGAGAUUUUUUUU-3′ (SEQ ID NO: 46).

In some embodiments, the sgRNA for use with SinCas9 comprises a scaffold comprising a sequence having at least 80% identity to 5′-GUUUUUGUACUCUCAAGAUUUGAAAAAAUCUUGCAGAAGCUACAAAGAUAAGG CUUCAUGCCGAAAUCAACACCCUGUCUAUGACGGGGUGUUUU-3′ (SEQ ID NO: 47).

In some embodiments, the sgRNA for use with SsaCas9 comprises a scaffold comprising a sequence having at least 80% identity to 5′-GUUUUAGAGCUGUGUUGUGAAAACAACACAGCAAGUUAAAAUAAGGCUUUGUC CGUACACAACUUGAAAAAGUGCGCACCGAUUCGGUGCUUUUUU-3′ (SEQ ID NO: 48).

In some embodiments, the sgRNA for use with Ssc2Cas9 comprises a scaffold comprising a sequence having at least 80% identity to 5′-GUUUUUGUACUCUCAAGAUUUGAAAAAAUCUUGCAGAAGCUACAAAGAUAAGG CUUCAUGCCGAAAUCAACACCCUGUCAUUUUAUGGCAGGGUGUUUU-3′ (SEQ ID NO: 49).

In some embodiments, the sgRNA for use with Sor2Cas9 comprises a scaffold comprising a sequence having at least 80% identity to 5′-GUUUUUGUACUCUCAAGAUUUGAAAAAAUCUUGCAGAAGCUACAAAGAUAAGG CUUCAUGCCGAAUUCAACACCCUGUCAUUUAUGGCGGGGUGUUUU-3′ (SEQ ID NO: 50).

In some embodiments, the sgRNA for use with SorCas9 comprises a scaffold comprising a sequence having at least 80% identity to 5′-GUUUUUGUACUCUCAAGAUUUGAAAAAAUCUUGUAGAAGCUACAAAGAUAAGG CUUCAUGCCGAAUUCAACACCCUGUCAUUUAUGGCGGGGUGUUUCGUUUU-3′ (SEQ ID NO: 51).

In some embodiments, the sgRNA for use with SwaCas9 comprises a scaffold comprising a sequence having at least 80% identity to 5′-GUUUUAGUACUCUGUAAUUUUGAAAAAAAUUACAGAAUCUACUGAAACAAGAC UAUAUGUCGUGUUUAUCCCACUAAUUUAUUAGUGGGAUUUUUU-3′ (SEQ ID NO: 52).

In some embodiments, the sgRNA for use with SscCas9 comprises a scaffold comprising a sequence having at least 80% identity to 5′-GUUUUAGUACUCUGUAAUUUUGAAAAAAAUUACAGAAUCUACUAAAACAAGGC AAAAUGCCGUGUUUAUCUCGUCAACUUGUUGACGAGAUUUUUUU-3′ (SEQ ID NO: 53).

In some embodiments, the sgRNA for use with SgaCas9 comprises a scaffold comprising a sequence having at least 80% identity to 5′-5′-GUUUUUGUACUCUCAAGAUUUGAAAAAAUCUUGCUGAGCCUACAAAGAUAAGG CUUUAUGCCGAAUUCAAGCACCCCAUGUUUUGACAUGGGGUGCUUUU-3′ (SEQ ID NO: 54).

In some embodiments, the sgRNA for use with LkuCas9 comprises a scaffold comprising a sequence having at least 80% identity to 5′-5′-GUUUUAGAUGAUUGUUAGAUCGAAAGAUCUAACAACCAGAUUUUAAAAUCAAA CAAUGUAUCUUUGAUACUAAGUUUCAACGCGGUAUUAUUACCGUCCUGCCUCA GCUCUAUAGCGGAGGUUUUUU-3′ (SEQ ID NO: 55).

In some embodiments, the sgRNA for use with SsuCas9 comprises a scaffold comprising a sequence having at least 80% identity to 5′-5′-GUUUUUGUACUCUCAAGAUUUGAAAAAAUCUUGCAGAGCCUACAAAGAUAAG GCUUUAUGCCGAAAUCAAGCACCCCGUUUCGUACGGGGUGCUUUU-3′ (SEQ ID NO: 56).

In the preceding embodiments, for SEQ ID NOs 43-56: Direct repeat (italics and underlined), tetraloop (italics), tracrRNA (underlined)

In some embodiments, the crRNA comprises a guide sequence of between about 16 and 26 nucleotides long.

In some embodiments, the crRNA comprises a guide sequence between 18 and 24 nucleotides long.

In some embodiments, the break in the target nucleic acid is a single-stranded or double-stranded break.

In some embodiments, the break in the target nucleic acid is a single-stranded break.

In some embodiments, the Cas9 protein is a nuclease that cleaves both strands of the target nucleic acid sequence. In some embodiments, the Cas9 is a nickase that cleaves one strand of the target nucleic acid sequence.

In some embodiments, the target nucleic acid is 5′ to a protospacer adjacent motif (PAM) sequence.

In some embodiments, the Cas9 is operably linked to a promoter sequence for expression in a eukaryotic cell, and wherein the guide RNA is operably linked to a promoter sequence for expression in a eukaryotic cell.

In some embodiments, the eukaryotic cell is a human cell.

In some embodiments, the promoter sequence is a eukaryotic or viral promoter.

In one aspect, provided herein is an engineered, non-naturally occurring CRISPR-Cas system comprising: an RNA guide or a nucleic acid encoding the RNA guide, wherein the RNA guide comprises a direct repeat sequence and a spacer sequence capable of hybridizing to a target nucleic acid; and a codon-optimized CRISPR-associated (Cas) protein having at least 80% sequence identity to SEQ ID NOs: 1, 2, 3, 4, 5, 6, 7, 8, 9, 10, 11, 12, 13 or 14, and wherein the Cas protein is capable of binding to the RNA guide and of causing a break in the target nucleic acid sequence complementary to the RNA guide.

In one aspect, provided herein is an engineered, non-naturally occurring CRISPR-Cas system comprising: an RNA guide or a nucleic acid encoding the RNA guide, wherein the RNA guide comprises a direct repeat sequence and a spacer sequence capable of hybridizing to a target nucleic acid; and a codon-optimized CRISPR-associated (Cas) protein having at least 80% sequence identity to SEQ ID NOs: 1, 2, 3, 4, 5, 6, 7, 8, 9, 10, 11, 12, 13 or 14; wherein the Cas protein is fused to a deaminase, and wherein the Cas protein fusion is capable of binding to the RNA guide and of editing the target nucleic acid sequence complementary to the RNA guide.

In one embodiment, the Cas9 protein is an inactive Cas9 (dCas9).

In one embodiment, the RNA guide comprises a crRNA and a tracrRNA.

In one embodiment, the RNA guide comprises an sgRNA.

In one embodiment, the Cas protein is operably linked to a promoter sequence for expression in a eukaryotic cell, and wherein the guide RNA is operably linked to a promoter sequence for expression in a eukaryotic cell.

In one embodiment, the eukaryotic cell is a human cell.

In one embodiment, the promoter sequence is a eukaryotic promoter sequence.

In one embodiment, a nucleic acid encoding the system described herein is provided.

In one embodiment, a vector comprising the system described herein is provided.

In one embodiment, the vector is a plasmid vector or a viral vector.

In one embodiment, the viral vector is an adeno associated virus (AAV) vector or a lentiviral vector.

In one embodiment, the viral vector is an AAV vector.

In one embodiment, more than one AAV vector is used for packaging the system.

In one embodiment, a method of treating a disorder or a disease in a subject in need thereof comprises administering to the subject the system described herein, wherein the guide RNA is complementary to at least 10 nucleotides of a target nucleic acid associated with the condition or disease; wherein the Cas protein associates with the guide RNA; wherein the guide RNA binds to the target nucleic acid; wherein the Cas protein causes a break in the target nucleic acid, optionally wherein the Cas9 is an inactive Cas9 (dCas9) fused to a deaminase and results in one or more base edits in the target nucleic acid, thereby treating the disorder or disease.

In some embodiments, the guide RNA is complementary to about 18-24 nucleotides.

In some embodiments, the guide RNA is complementary to 20 nucleotides.

In some embodiments, the base editor comprises a fusion protein.

In some embodiments, the base editor comprises an adenosine deaminase domain or a cytidine deaminase domain.

In some embodiments, provided herein is a method of editing a nucleobase of a polynucleotide, the method comprising contacting the polynucleotide with a base in complex with one or more guide RNAs, wherein the base editor comprises an adenosine deaminase domain, and wherein the one or more guide RNAs target the base editor to effect an A⋅T to G⋅C alteration in the polynucleotide.

In some embodiments, provided herein is a method of editing a nucleobase of a polynucleotide, the method comprising contacting the polynucleotide with a base editor in complex with one or more guide RNAs, wherein the base editor comprises a cytidine deaminase domain, and wherein the one or more guide RNAs target the base editor to effect a C⋅G to T⋅A alteration in the polynucleotide.

In some embodiments, the editing results in less than 50% indel formation in the target polynucleotide sequence.

In some embodiments, the editing generates a point mutation.

### Definitions

In order for the present invention to be more readily understood, certain terms are first defined below. Additional definitions for the following terms and other terms are set forth throughout the specification.

A or An: The articles “a” and “an” are used herein to refer to one or to more than one (i.e., to at least one) of the grammatical object of the article. By way of example, “an element” means one element or more than one element.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: Two events or entities are “associated” with one another, as that term is used herein, if the presence, level and/or form of one is correlated with that of the other. For example, a particular entity (e.g., polypeptide) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Base Editor: By “base editor (BE),” or “nucleobase editor (NBE)” is meant an agent that binds a polynucleotide and has nucleobase modifying activity. In various embodiments, the base editor comprises a nucleobase modifying polypeptide (e.g., a deaminase) and a polynucleotide programmable nucleotide binding domain in conjunction with a guide polynucleotide (e.g., guide RNA). In various embodiments, the agent is a biomolecular complex comprising a protein domain having base editing activity, i.e., a domain capable of modifying a base (e.g., A, T, C, G, or U) within a nucleic acid molecule (e.g., DNA). In some embodiments, the polynucleotide programmable DNA binding domain is fused or linked to a deaminase domain. In one embodiment, the agent is a fusion protein comprising one or more domains having base editing activity. In another embodiment, the protein domains having base editing activity are linked to the guide RNA (e.g., via an RNA binding motif on the guide RNA and an RNA binding domain fused to the deaminase). In some embodiments, the domains having base editing activity are capable of deaminating a base within a nucleic acid molecule. In some embodiments, the base editor is capable of deaminating one or more bases within a DNA molecule. In some embodiments, the base editor is capable of deaminating a cytosine (C) or an adenosine (A) within DNA. In some embodiments, the base editor is capable of deaminating a cytosine (C) and an adenosine (A) within DNA. In some embodiments, the base editor is a cytidine base editor (CBE). In some embodiments, the base editor is an adenosine base editor (ABE). In some embodiments, the base editor is an adenosine base editor (ABE) and a cytidine base editor (CBE). In some embodiments, the base editor is a nuclease-inactive Cas9 (dCas9) fused to an adenosine deaminase. In some embodiments, the base editor is fused to an inhibitor of base excision repair, for example, a UGI domain, or a dISN domain. In some embodiments, the fusion protein comprises a Cas9 nickase fused to a deaminase and an inhibitor of base excision repair, such as a UGI or dISN domain. In other embodiments the base editor is an abasic base editor. Details of base editors are described in International PCT Application Nos. PCT/2017/045381 (WO2018/027078) and PCT/US2016/058344 (WO2017/070632), each of which is incorporated herein by reference for its entirety. Also see Komor, A. C., et al., “Programmable editing of a target base in genomic DNA without double-stranded DNA cleavage” Nature 533, 420-424 (2016); Gaudelli, N. M., et al., “Programmable base editing of A⋅T to G⋅C in genomic DNA without DNA cleavage” Nature 551, 464-471 (2017); Komor, A. C., et al., “Improved base excision repair inhibition and bacteriophage Mu Gam protein yields C:G-to-T:A base editors with higher efficiency and product purity” Science Advances 3:eaao4774 (2017), and Rees, H. A., et al., “Base editing: precision chemistry on the genome and transcriptome of living cells.” Nat Rev Genet. 2018 December; 19(12):770-788. doi: 10.1038/s41576-018-0059-1, the entire contents of which are hereby incorporated by reference.

Base Editing Activity: By “base editing activity” is meant acting to chemically alter a base within a polynucleotide. In one embodiment, a first base is converted to a second base. In one embodiment, the base editing activity is cytidine deaminase activity, e.g., converting target C⋅G to T⋅A. In another embodiment, the base editing activity is adenosine or adenine deaminase activity, e.g., converting A⋅T to G⋅C. In another embodiment, the base editing activity is cytosine or cytidine deaminase activity, e.g., converting target C⋅G to T⋅A and adenosine or adenine deaminase activity, e.g., converting A⋅T to G⋅C.

Base Editor System: The term “base editor system” refers to a system for editing a nucleobase of a target nucleotide sequence. In various embodiments, the base editor (BE) system comprises (1) a polynucleotide programmable nucleotide binding domain (e.g., Cas9), a deaminase domain and a cytidine deaminase domain for deaminating nucleobases in the target nucleotide sequence; and (2) one or more guide polynucleotides (e.g., guide RNA) in conjunction with the polynucleotide programmable nucleotide binding domain. In various embodiments, the base editor (BE) system comprises a nucleobase editor domains selected from an adenosine deaminase or a cytidine deaminase, and a domain having nucleic acid sequence specific binding activity. In some embodiments, the base editor system comprises (1) a base editor (BE) comprising a polynucleotide programmable DNA binding domain and a deaminase domain for deaminating one or more nucleobases in a target nucleotide sequence; and (2) one or more guide RNAs in conjunction with the polynucleotide programmable DNA binding domain. In some embodiments, the polynucleotide programmable nucleotide binding domain is a polynucleotide programmable DNA binding domain. In some embodiments, the base editor is a cytidine base editor (CBE). In some embodiments, the base editor is an adenine or adenosine base editor (ABE). In some embodiments, the base editor is an adenine or adenosine base editor (ABE) or a cytidine base editor (CBE).

In some embodiments, a polynucleotide programmable nucleotide binding domain can target a deaminase domain to a target nucleotide sequence by non-covalently interacting with or associating with the deaminase domain. For example, in some embodiments, the nucleobase editing component, e.g., the deaminase component can comprise an additional heterologous portion or domain that is capable of interacting with, associating with, or capable of forming a complex with an additional heterologous portion or domain that is part of a polynucleotide programmable nucleotide binding domain. In some embodiments, the additional heterologous portion may be capable of binding to, interacting with, associating with, or forming a complex with a polypeptide. In some embodiments, the additional heterologous portion may be capable of binding to, interacting with, associating with, or forming a complex with a polynucleotide. In some embodiments, the additional heterologous portion may be capable of binding to a guide polynucleotide. In some embodiments, the additional heterologous portion may be capable of binding to a polypeptide linker. In some embodiments, the additional heterologous portion may be capable of binding to a polynucleotide linker. The additional heterologous portion may be a protein domain. In some embodiments, the additional heterologous portion may be a K Homology (KH) domain, a MS2 coat protein domain, a PP7 coat protein domain, a SfMu Com coat protein domain, a steril alpha motif, a telomerase Ku binding motif and Ku protein, a telomerase Sm7 binding motif and Sm7 protein, or an RNA recognition motif.

Biologically active: As used herein, the phrase “biologically active” refers to a characteristic of any agent that has activity in a biological system, and particularly in an organism. For instance, an agent that, when administered to an organism, has a biological effect on that organism, is considered to be biologically active. In particular embodiments, where a peptide is biologically active, a portion of that peptide that shares at least one biological activity of the peptide is typically referred to as a “biologically active” portion.

Cleavage: As used herein, cleavage refers to a break in a target nucleic acid created by a nuclease of a CRISPR system described herein. In some embodiments, the cleavage event is a double-stranded DNA break. In some embodiments, the cleavage event is a single-stranded DNA break. In some embodiments, the cleavage event is a single-stranded RNA break. In some embodiments, the cleavage event is a double-stranded RNA break.

Complementary: As used herein, complementary refers to a nucleic acid strand that forms Watson-Crick base pairing, such that A base pairs with T, and C base pairs with G, or non-traditional base pairing with bases on a second nucleic acid strand. In other words, it refers to nucleic acids that hybridize with each other under appropriate conditions.

Clustered Interspaced Short Palindromic Repeat (CRISPR)-associated (Cas) system: As used herein, CRISPR-Cas9 system refers to nucleic acids and/or proteins involved in the expression of, or directing the activity of, CRISPR-effectors, including sequences encoding CRISPR effectors, RNA guides, and other sequences and transcripts from a CRISPR locus. In some embodiments, the CRISPR system is an engineered, non-naturally occurring CRISPR system. In some embodiments, the components of a CRISPR system may include a nucleic acid(s) (e.g., a vector) encoding one or more components of the system, a component(s) in protein form, or a combination thereof.

CRISPR Array: The term “CRISPR array”, as used herein, refers to the nucleic acid (e.g., DNA) segment that includes CRISPR repeats and spacers, starting with the first nucleotide of the first CRISPR repeat and ending with the last nucleotide of the last (terminal) CRISPR repeat. Typically, each spacer in a CRISPR array is located between two repeats. The terms “CRISPR repeat” or “CRISPR direct repeat,” or “direct repeat,” as used herein, refer to multiple short direct repeating sequences, which show very little or no sequence variation within a CRISPR array.

CRISPR-associated protein (Cas): The term “CRISPR-associated protein,” “CRISPR effector,” “effector,” or “CRISPR enzyme” as used herein refers to a protein that carries out an enzymatic activity or that binds to a target site on a nucleic acid specified by a RNA guide. In different embodiments, a CRISPR effector has endonuclease activity, nickase activity, exonuclease activity, transposase activity, and/or excision activity. It should be appreciated that any of the Cas9 proteins provided herein include naturally occurring Cas9 proteins and non-naturally occurring variants thereof.

crRNA: The term “CRISPR RNA” or “crRNA,” as used herein, refers to a RNA molecule including a guide sequence used by a CRISPR effector to target a specific nucleic acid sequence. Typically, crRNAs contains a sequence that mediates target recognition and a sequence that forms a duplex with a tracrRNA. In some embodiments, the crRNA: tracrRNA duplex binds to a CRISPR effector.

Ex Vivo: As used herein, the term “ex vivo” refers to events that occur in cells or tissues, grown outside rather than within a multi-cellular organism.

Functional equivalent or analog: As used herein, the term “functional equivalent” or “functional analog” denotes, in the context of a functional derivative of an amino acid sequence, a molecule that retains a biological activity (either function or structural) that is substantially similar to that of the original sequence. A functional derivative or equivalent may be a natural derivative or is prepared synthetically. Exemplary functional derivatives include amino acid sequences having substitutions, deletions, or additions of one or more amino acids, provided that the biological activity of the protein is conserved. The substituting amino acid desirably has chemico-physical properties which are similar to that of the substituted amino acid. Desirable similar chemico-physical properties include, similarities in charge, bulkiness, hydrophobicity, hydrophilicity, and the like.

Half-Life: As used herein, the term “half-life” is the time required for a quantity such as protein concentration or activity to fall to half of its value as measured at the beginning of a time period.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein. A “control subject” is a subject afflicted with the same form of disease as the subject being treated, who is about the same age as the subject being treated.

Inhibition: As used herein, the terms “inhibition,” “inhibit” and “inhibiting” refer to processes or methods of decreasing or reducing activity and/or expression of a protein or a gene of interest. Typically, inhibiting a protein or a gene refers to reducing expression or a relevant activity of the protein or gene by at least 10% or more, for example, 20%, 30%, 40%, or 50%, 60%, 70%, 80%, 90% or more, or a decrease in expression or the relevant activity of greater than 1-fold, 2-fold, 3-fold, 4-fold, 5-fold, 10-fold, 50-fold, 100-fold or more as measured by one or more methods described herein or recognized in the art.

Hybridization: As used herein, the term “hybridization” refers to a reaction in which two or more nucleic acids bind with each other via hydrogen bonding by Watson-Crick pairing, Hoogstein binding or other sequence-specific binding between the bases of the two nucleic acids. A sequence capable of hybridizing with another sequence is termed the “complement” of the sequence, and is said to be “complementary” or show “complementarity”.

Indel: As used herein, the term “indel” refers to insertion or deletion of bases in a nucleic acid sequence. It commonly results in mutations and is a common form of genetic variation.

In Vitro: As used herein, the term “in vitro” refers to events that occur in an artificial environment, e.g., in a test tube or reaction vessel, in cell culture, etc., rather than within a multi-cellular organism.

In Vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Linker: The term “linker” refers to any means, entity or moiety used to join two or more entities. In some embodiments, the linker is a covalent linker. In some embodiments, the linker is a non-covalent linker, Examples of covalent linkers include covalent bonds or a linker moiety covalently attached to one or more of the proteins or domains to be linked. In some embodiments, the linker is a non-covalent bond. e.g., an organometallic bond through a metal center such as platinum atom. The joining can be permanent or reversible. For covalent linkages, various functionalities can be used, such as amide groups, including carbonic acid derivatives, ethers, esters, including organic and inorganic esters, amino, urethane, urea and the like. To provide for linking, the domains can be modified by oxidation, hydroxylation, substitution, reduction etc. to provide a site for coupling. Methods for conjugation are well known by persons skilled in the art and are encompassed for use in the present invention. Linker moieties include, but are not limited to, chemical linker moieties, or for example a peptide linker moiety (a linker sequence. It will be appreciated that modification which do not significantly decrease the function of the RNA-binding domain and effector domain are preferred.

Mutation: As used herein, the term “mutation” has the ordinary meaning in the art, and includes, for example, point mutations, substitutions, insertions, deletions, inversions, and deletions.

Oligonucleotide: As used herein, the term “oligonucleotide” generally refers to polynucleotides of between about 5 and about 100 nucleotides of single- or double-stranded DNA. Oligonucleotides are also known as “oligomers” or “oligos” and may be isolated from genes, or chemically synthesized.

PAM: The term “PAM” or “Protospacer Adjacent Motif” refers to a short nucleic acid sequence (usually 2-6 base pairs in length) that follows the nucleic acid region targeted for cleavage by the CRISPR system, such as CRISPR-Cas9. The PAM is required for a Cas nuclease to cut and is generally found 3-4 nucleotides downstream from the cut site.

Polypeptide: The term “polypeptide” as used herein refers to a sequential chain of amino acids linked together via peptide bonds. The term is used to refer to an amino acid chain of any length, but one of ordinary skill in the art will understand that the term is not limited to lengthy chains and can refer to a minimal chain comprising two amino acids linked together via a peptide bond. As is known to those skilled in the art, polypeptides may be processed and/or modified. As used herein, the terms “polypeptide” and “peptide” are used inter-changeably.

Prevent: As used herein, the term “prevent” or “prevention”, when used in connection with the occurrence of a disease, disorder, and/or condition, refers to reducing the risk of developing the disease, disorder and/or condition.

Protein: The term “protein” as used herein refers to one or more polypeptides that function as a discrete unit. If a single polypeptide is the discrete functioning unit and does not require permanent or temporary physical association with other polypeptides in order to form the discrete functioning unit, the terms “polypeptide” and “protein” may be used interchangeably. If the discrete functional unit is comprised of more than one polypeptide that physically associate with one another, the term “protein” refers to the multiple polypeptides that are physically coupled and function together as the discrete unit.

Reference: A “reference” entity, system, amount, set of conditions, etc., is one against which a test entity, system, amount, set of conditions, etc. is compared as described herein. For example, in some embodiments, a “reference” antibody is a control antibody that is not engineered as described herein.

RNA guide: The term RNA guide refers to an RNA molecule that facilitates the targeting of a protein described herein to a target nucleic acid. Exemplary “RNA guides” or “guide RNAs” include, but are not limited to, crRNAs or crRNAs in combination with cognate tracrRNAs. The latter may be independent RNAs or fused as a single RNA using a linker (sgRNAs). In some embodiments, the RNA guide is engineered to include a chemical or biochemical modification, in some embodiments, an RNA guide may include one or more nucleotides.

Subject: The term “subject”, as used herein, means any subject for whom diagnosis, prognosis, or therapy is desired. For example, a subject can be a mammal, e.g., a human or non-human primate (such as an ape, monkey, orangutan, or chimpanzee), a dog, cat, guinea pig, rabbit, rat, mouse, horse, cattle, or cow.

sgRNA: The term “sgRNA” or “single guide RNA” refers to a single guide RNA containing (i) a guide sequence (crRNA sequence) and (ii) a Cas9 nuclease-recruiting sequence (tracrRNA).

Substantial identity: The phrase “substantial identity” is used herein to refer to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., Basic local alignment search tool, J. Mol. Biol., 215(3): 403-410, 1990; Altschul, et al., Methods in Enzymology; Altschul et al., Nucleic Acids Res. 25:3389-3402, 1997; Baxevanis et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying identical sequences, the programs mentioned above typically provide an indication of the degree of identity. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues.

Target Nucleic Acid: The term “target nucleic acid” as used herein refers to nucleotides of any length (oligonucleotides or polynucleotides) to which the CRISPR-Cas9 system binds, either deoxyribonucleotides, ribonucleotides, or analogs thereof. Target nucleic acids may have three-dimensional structure, may including coding or non-coding regions, may include exons, introns, mRNA, tRNA, rRNA, siRNA, shRNA, miRNA, ribozymes, cDNA, plasmids, vectors, exogenous sequences, endogenous sequences. A target nucleic acid can comprise modified nucleotides, include methylated nucleotides, or nucleotide analogs. A target nucleic acid may be interspersed with non-nucleic acid components. A target nucleic acid is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

Therapeutically effective amount: As used herein, the term “therapeutically effective amount” refers to an amount of a therapeutic molecule (e.g., an engineered antibody described herein) which confers a therapeutic effect on a treated subject, at a reasonable benefit/risk ratio applicable to any medical treatment. The therapeutic effect may be objective (i.e., measurable by some test or marker) or subjective (i.e., subject gives an indication of or feels an effect). In particular, the “therapeutically effective amount” refers to an amount of a therapeutic molecule or composition effective to treat, ameliorate, or prevent a particular disease or condition, or to exhibit a detectable therapeutic or preventative effect, such as by ameliorating symptoms associated with the disease, preventing or delaying the onset of the disease, and/or also lessening the severity or frequency of symptoms of the disease. A therapeutically effective amount can be administered in a dosing regimen that may comprise multiple unit doses. For any particular therapeutic molecule, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular subject may depend upon a variety of factors including the disorder being treated and the severity of the disorder; the activity of the specific pharmaceutical agent employed; the specific composition employed; the age, body weight, general health, sex and diet of the subject; the time of administration, route of administration, and/or rate of excretion or metabolism of the specific therapeutic molecule employed; the duration of the treatment; and like factors as is well known in the medical arts.

tracrRNA: The term “tracrRNA” or “trans-activating crRNA” as used herein refers to an RNA including a sequence that forms a structure required for a CRISPR-associated protein to bind to a specified target nucleic acid.

Treatment: As used herein, the term “treatment” (also “treat” or “treating”) refers to any administration of a therapeutic molecule (e.g., a CRISPR-Cas therapeutic protein or system described herein) that partially or completely alleviates, ameliorates, relieves, inhibits, delays onset of, reduces severity of and/or reduces incidence of one or more symptoms or features of a particular disease, disorder, and/or condition. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition.

### BRIEF DESCRIPTION OF THE DRAWING

Drawings are for illustration purposes only; not for limitation.

FIG. 1A is a graph that shows a consensus PAM motif recognized by human codon-optimized Streptococcus equinus ATCC 33317 Cas9. FIG. 1B is a graph that shows a consensus PAM motif recognized by human codon-optimized Enterococcus hirae strain F1129E Cas9. FIG. 1C is a graph that shows a consensus PAM motif recognized by human codon-optimized Streptococcus equinus strain AG46 Cas9. FIG. 1D is a graph that shows a consensus PAM motif recognized by human codon-optimized Staphylococcus simulans strain 19. FIG. 1E is a graph that shows a consensus PAM motif recognized by human codon-optimized Streptococcus intermedius B196 strain G1552. FIG. 1F is a graph that shows a consensus PAM motif recognized by human codon-optimized Streptococcus sanguinis SK330. FIG. 1G is a graph that shows a consensus PAM motif recognized by human codon-optimized Streptococcus sp. C150. FIG. 1H is a graph that shows a consensus PAM motif recognized by human codon-optimized Streptococcus oralis subsp. oralis strain RH_1735_08. FIG. 1I is a graph that shows a consensus PAM motif recognized by Streptococcus oralis SK313. FIG. 1J is a graph that shows a consensus PAM motif recognized by Staphylococcus warneri strain 691. FIG. 1K is a graph that shows a consensus PAM motif recognized by Staphylococcus sciuri strain SNUC 2430. FIG. 1L is a graph that shows a consensus PAM motif recognized by Streptococcus gallolyticus strain AM24-4. FIG. 1M is a graph that shows a consensus PAM motif recognized by Lactobacillus kullabergensis strain Biut2. FIG. iN is a graph that shows a consensus PAM motif recognized by Streptococcus suis strain (N=A, T, G or C; H=A, C or T; R=A or G).

FIG. 2A is a schematic that shows predicted RNA folding structure of sgRNA for human codon-optimized Streptococcus equinus ATCC 33317 (Seq2Cas9) using Geneious software. FIG. 2A depicts sgRNA comprising SEQ ID NO:198. FIG. 2B is a schematic that shows predicted RNA folding structure of sgRNA for human codon-optimized Enterococcus hirae strain F1129E Cas9 (EhiCas9) using Geneious software. FIG. 2B depicts sgRNA comprising SEQ ID NO: 44. FIG. 2C is a schematic that shows predicted RNA folding structure of sgRNA for human codon-optimized Streptococcus equinus strain AG46 Cas9 (SeqCas9) using Geneious software. FIG. 2C depicts sgRNA comprising SEQ ID NO: 46. FIG. 2D is a schematic that shows predicted RNA folding structure of sgRNA for human codon-optimized Staphylococcus simulans strain 19 (SsiCas9) using Geneious software. FIG. 2D depicts sgRNA comprising SEQ ID NO: 45. FIG. 2E is a schematic that shows predicted RNA folding structure of sgRNA for human codon-optimized Streptococcus intermedius B196 strain G1552 (SinCas9) using Geneious software. FIG. 2E depicts sgRNA comprising SEQ ID NO: 47. FIG. 2F is a schematic that shows predicted RNA folding structure of sgRNA for human codon-optimized Streptococcus sanguinis SK330 (SsaCas9) using Geneious software. FIG. 2F depicts sgRNA comprising SEQ ID NO: 48. FIG. 2G is a schematic that shows predicted RNA folding structure of sgRNA for human codon-optimized Streptococcus sp. C150 (Ssc2Cas9) using Geneious software. FIG. 2G depicts sgRNA comprising SEQ ID NO: 49. FIG. 2H is a schematic that shows predicted RNA folding structure of sgRNA for human codon-optimized Streptococcus oralis subsp. oralis strain RH_1735_08 (Sor2Cas9) using Geneious software. FIG. 2H depicts sgRNA comprising SEQ ID NO: 50. FIG. 2I is a schematic that shows predicted RNA folding structure of sgRNA for human codon-optimized Streptococcus oralis SK313 (SorCas9) using Geneious software. FIG. 2I depicts sgRNA comprising SEQ ID NO: 51. FIG. 2J is a schematic that shows predicted RNA folding structure of sgRNA for human codon-optimized Staphylococcus warneri strain 691 (SwaCas9) using Geneious software. FIG. 2J depicts sgRNA comprising SEQ ID NO: 52. FIG. 2K is a schematic that shows predicted RNA folding structure of sgRNA for human codon-optimized Staphylococcus sciuri strain SNUC 2430 using Geneious software. FIG. 2K depicts sgRNA comprising SEQ ID NO: 53. FIG. 2L is a schematic that shows predicted RNA folding structure of sgRNA for human codon-optimized Streptococcus gallolyticus strain AM24-4 (SgaCas9) using Geneious software. FIG. 2L depicts sgRNA comprising SEQ ID NO: 54. FIG. 2M is a schematic that shows predicted RNA folding structure of sgRNA for human codon-optimized Lactobacillus kullabergensis strain Biut2 (LkuCas9) using Geneious software. FIG. 2M depicts sgRNA comprising SEQ ID NO: 55. FIG. 2N is a schematic that shows predicted RNA folding structure of sgRNA for human codon-optimized Streptococcus suis strain LSS83 using Geneious software. FIG. 2N depicts sgRNA comprising SEQ ID NO: 56.

FIG. 3A is a graph that shows results of adenine to guanine base (A-to-G) conversion percentage achieved with a base editor comprising an ABE fused to the N-terminus of a Seq2Cas9 D10A mutant. The A-to-G conversion percentage (y-axis) is plotted for various guide RNAs targeting A-rich genomic test sites (x-axis; Table 8) adjacent to a sequence corresponding to the PAM consensus motif (see FIG. 1A).

FIG. 3B is a graph that shows results of adenine to guanine base (A-to-G) conversion percentage achieved with a base editor comprising an ABE fused to the N-terminus of a EhiCas9 D10A mutant. The A-to-G conversion percentage (y-axis) is plotted for various guide RNAs targeting A-rich genomic test sites (x-axis; Table 9) adjacent to a sequence corresponding to the PAM consensus motif (see FIG. 1B).

FIG. 3C is a graph that shows results of adenine to guanine base (A-to-G) conversion percentage achieved with a base editor comprising an ABE fused to the N-terminus of a SeqCas9 D10A mutant. The A-to-G conversion percentage (y-axis) is plotted for various guide RNAs targeting A-rich genomic test sites (x-axis; Table 10) adjacent to a sequence corresponding to the PAM consensus motif (see FIG. 1C).

FIG. 3D is a graph that shows results of adenine to guanine base (A-to-G) conversion percentage achieved with a base editor comprising an ABE fused to the N-terminus of a SsiCas9 D10A mutant. The A-to-G conversion percentage (y-axis) is plotted for various guide RNAs targeting A-rich genomic test sites (x-axis; Table 11) adjacent to a sequence corresponding to the PAM consensus motif (see FIG. 1D).

FIG. 3E is a graph that shows results of adenine to guanine base (A-to-G) conversion percentage achieved with a base editor comprising an ABE fused to the N-terminus of a SinCas9 D9A mutant. The A-to-G conversion percentage (y-axis) is plotted for various guide RNAs targeting A-rich genomic test sites (x-axis; Table 12) adjacent to a sequence corresponding to the PAM consensus motif (see FIG. 1E).

FIG. 3F is a graph that shows results of adenine to guanine base (A-to-G) conversion percentage achieved with a base editor comprising an ABE fused to the N-terminus of a SsaCas9 D11A mutant. The A-to-G conversion percentage (y-axis) is plotted for various guide RNAs targeting A-rich genomic test sites (x-axis; Table 13) adjacent to a sequence corresponding to the PAM consensus motif (see FIG. 1F).

FIG. 3G is a graph that shows results of adenine to guanine base (A-to-G) conversion percentage achieved with a base editor comprising an ABE fused to the N-terminus of a Ssc2Cas9 D9A mutant. The A-to-G conversion percentage (y-axis) is plotted for various guide RNAs targeting A-rich genomic test sites (x-axis; Table 14) adjacent to a sequence corresponding to the PAM consensus motif (see FIG. 1G).

FIG. 3H is a graph that shows results of adenine to guanine base (A-to-G) conversion percentage achieved with a base editor comprising an ABE fused to the N-terminus of a Sor2Cas9 D9A mutant. The A-to-G conversion percentage (y-axis) is plotted for various guide RNAs targeting A-rich genomic test sites (x-axis; Table 15) adjacent to a sequence corresponding to the PAM consensus motif (see FIG. 1H).

FIG. 3I is a graph that shows results of adenine to guanine base (A-to-G) conversion percentage achieved with a base editor comprising an ABE fused to the N-terminus of a SorCas9 D9A mutant. The A-to-G conversion percentage (y-axis) is plotted for various guide RNAs targeting A-rich genomic test sites (x-axis; Table 16) adjacent to a sequence corresponding to the PAM consensus motif (see FIG. 1I).

FIG. 3J is a graph that shows results of adenine to guanine base (A-to-G) conversion percentage achieved with a base editor comprising an ABE fused to the N-terminus of a SwaCas9 mutant. The A-to-G conversion percentage (y-axis) is plotted for various guide RNAs targeting A-rich genomic test sites (x-axis; Table 17) adjacent to a sequence corresponding to the PAM consensus motif (see FIG. 1J).

FIG. 3K is a graph that shows results of adenine to guanine base (A-to-G) conversion percentage achieved with a base editor comprising an ABE fused to the N-terminus of a SscCas9 mutant. The A-to-G conversion percentage (y-axis) is plotted for various guide RNAs targeting A-rich genomic test sites (x-axis; Table 18) adjacent to a sequence corresponding to the PAM consensus motif (see FIG. 1K).

FIG. 3L is a graph that shows results of adenine to guanine base (A-to-G) conversion percentage achieved with a base editor comprising an ABE fused to the N-terminus of a SgaCas9 mutant. The A-to-G conversion percentage (y-axis) is plotted for various guide RNAs targeting A-rich genomic test sites (x-axis; Table 19) adjacent to a sequence corresponding to the PAM consensus motif (see FIG. 1L).

FIG. 3M is a graph that shows results of adenine to guanine base (A-to-G) conversion percentage achieved with a base editor comprising an ABE fused to the N-terminus of a LkuCas9 mutant. The A-to-G conversion percentage (y-axis) is plotted for various guide RNAs targeting A-rich genomic test sites (x-axis; Table 20) adjacent to a sequence corresponding to the PAM consensus motif (see FIG. 1M).

FIG. 3N is a graph that shows results of adenine to guanine base (A-to-G) conversion percentage achieved with a base editor comprising an ABE fused to the N-terminus of a SsuCas9 mutant. The A-to-G conversion percentage (y-axis) is plotted for various guide RNAs targeting A-rich genomic test sites (x-axis; Table 21) adjacent to a sequence corresponding to the PAM consensus motif (see FIG. 1N).

### DETAILED DESCRIPTION

Clustered regularly interspaced short palindromic repeats (CRISPR) was first discovered as an adaptive immune system in bacteria and archaea, and then engineered to generate targeted DNA breaks in living cells and organisms. During the cellular DNA repair process, various DNA changes can be introduced. The diverse and expanding CRISPR toolbox allows programmable genome editing, epigenome editing and transcriptome regulation.

CRISPR-Cas systems comprise three main types (I, II, and III) based on their Cas gene organization, and the sequence and structure of component proteins. Each of the three CRISPR systems is characterized by a unique Cas gene: Cas3, a target-degrading nuclease/helicase in Type I; Cas9, an RNA-binding and target-degrading nuclease in type II; CasIO, a large protein for multiple functions in type III. The three CRISPR types also differ in their associated effector complexes. Type I Cas systems associate with Cascade effector complexes, type II effector complexes consist of a single Cas9 and one or more RNA molecules, and type III interference complexes are further divided into type III-A (Csm complex targeting DNA) and type III-B (Cmr complex targeting RNA). Cas proteins are important components of effector complexes in all CRISPR-Cas systems.

Current genome editing technologies have focused on Class II CRISPR-Cas systems, which contain single-protein effector nucleases for DNA cleavage, specifically, Cas9, a dual-RNA-guided nuclease which requires both CRISPR RNA (crRNA) and tracrRNA and contains both HNH and RuvC nuclease domains, and Cas12a, a single-RNA-guided nuclease which only requires crRNA and contains a single RuvC domain.

Various aspects of the invention are described in detail in the following sections. The use of sections is not meant to limit the invention. Each section can apply to any aspect of the invention. In this application, the use of “or” means “and/or” unless stated otherwise.

### Engineered, Non-Naturally Occurring Cas9 Protein

Described herein are engineered, non-naturally occurring Cas9 proteins modified from WT Cas9 obtained from Streptococcus equinus ATCC 33317 (Seq2Cas9), Enterococcus hirae strain F1129E (EhiCas9), Streptococcus equinus strain AG46 (SeqCas9), Staphylococcus simulans strain 19 (SsiCas9), Streptococcus intermedius B196 strain G1552 (SinCas9), Streptococcus sanguinis SK330 (SsaCas9), Streptococcus sp. C150 (Ssc2Cas9), Streptococcus oralis subsp. oralis strain RH_1735_08 (Sor2Cas9), Streptococcus oralis SK313 (SorCas9), Staphylococcus warneri strain 691 (SwaCas9), Staphylococcus sciuri strain SNUC 2430 (SscCas9), Streptococcus gallolyticus strain AM24-4 (SgaCas9), Lactobacillus kullabergensis strain Biut2 (LkuCas9), and Streptococcus suis strain LSS83 (SsuCas9).

In some embodiments, the engineered non-naturally occurring Cas9 protein described herein comprises an amino acid sequence at least 60% (e.g., 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or more) identical to SEQ ID NO: 1, 2, 3, 4, 5, 6, 7, 8, 9, 10, 11, 12, 13 or 14. In some embodiments, the Cas9 protein has is 80% identical to SEQ ID NO: 1, 2, 3, 4, 5, 6, 7, 8, 9, 10, 11, 12, 13 or 14. In some embodiments, the amino acid sequence of the Cas9 protein is identical to SEQ ID NO: 1, 2, 3, 4, 5, 6, 7, 8, 9, 10, 11, 12, 13 or 14. Exemplary Cas9 amino acid sequences are provided in Table 1 below.

TABLE 1
Exemplary Cas9 Amino Acid Sequences

Wild Type Streptococcus equinus ATCC 33317 Cas9 (Seq2Cas9)
MEKSYSIGLDIGTNSVGWSVITDDYKVPAKKMRVLGNTDKKYIKKNLLGALLFDS
GETAEATRLKRTARRRYTRRKNRLRYLQEIFAKEMAKVDESFFQRLDESFLTDDD
KTFDSHPIFGNKAEEDAYHQKFPTIYHLRKHLADSQEKADLRLIYLALAHMIKFRG
HFLYDNFNDDNFDWRNIDIQKRYEEFIETYDSTLGESYLADISVDAASILEEKVSKT
ERLENLLKYYPTEKKTTFFGNLIKLILGQQAKFKAIFNLEDEISLQFATPTYDEDLEE
LLGKIDNGDSYSELFVAAQNLYNTILLASFLKTDNKSAKAPLSTSMIERYENHKKD
LAKLKDFVKKNCPDQYHDIFRDKSKNGYAGYIDNGVSQNDFYTFIGKCLEESLKK
DKGAQYFLDKIDRDDFLRKQRTFDNGAFPYQIHLQEMHAILRRQGDYYPFLKENQ
DKIEKILTFRIPYYVGPLARKDSRFAWANYRSDEAITPWNFDEVVDKEKSAEKFITR
MTLNDLYLPEEKVLPKHSLIYETFTVYNELTNIKYVNDQGNAIHFDSELKEKIFNQL
FKENRKVSKKTLIDFLNNSEGIYTDKLVGIDEEVKYLNASLGTYHDLKKILESFMD
DEINEKIIEDIIQTLTLFEDIEMKRQRLQKYDDIFTPKQLKELARRNYTGWGRLSYK
LINGIRNKENNKTILDYLKNGNRNFMQLINDDRLSFKQIIIDARKIEKLDNIESVVYN
LPGSPAIKKGILQSIKIVDELVKVMGHNPDNIVIEMARENQTTNQGKNRSQQRLKR
LQDSMSNFKDSSISLKDVDNSDLQNDRLFLYYIQNGKDMYTGEELDIDHLSDYDID
HIIPQSFIKDNSIDNRVLTSSAKNRGKSDDVPGRDVVLKMKPFWKKLYDVKLISKR
KFDNLTKSEHGGLTESDKAGFIKRQLVETRQITKYVAQILDGRFNTKRDDNNKVIR
DVKVITLKSSLVSQFRKDFGFYKVREINDYHHAHDAYLNAVVGTAILKKYPKLAP
EFVYGEYKKCDVRKLIAKSGDKSEIGKATAKYFFYSNLMNFFKRVIRYSNGMIVV
RPVIEYSKDTGEIAWDKEKDFKTVCKVLSCPQVNIVKKVEKQSHGLDRGKPKGFY
NANPSPKPKKGSKVNLVPIKANLNPKNYGGYAGISNSYAVLVDATIEKGAKKKLT
RIQEFQGISIIDREKYEKNKVEFLKGLGYKEIYSIITLPKYSLFELADGSRRMLASILS
TNNKRGEIHKGNELVLPAKYIPLLYHANRIHNTFETGHREYVEKHIAEFKEIAEIILE
FNNKYVNAKKNSSIIEKALESFDSFSLDEICDSFVGKLKKNNTKKNSGLFELVSLGS
ASDFEFLETKVPRYRDYTPSSLLNATLIHQSITGLYETRIDLSKLGEE (SEQ ID NO:
1)
Wild Type Enterococcus hirae strain F1129E Cas9 (EhiCas9)
MTKDYTIGLDIGTNSVGWAVLTDDYQLMKRKMSVHGNTEKKKIKKNFWGARLF
DEGQTAEFRRTKRTNRRRLARRKYRLSKLQDLFAEELCKQDDCFFVRLEESFLVPE
EKQYKPASIFPTLEEEKEYYQKYPTIYHLRQKLVDSTEKEDLRLVYLALAHLLKYR
GHFLFEGDLDTENTSIEESFRVFLEQYSKQSDQPLIVHQPVLTILTDKLSKTKKVEEI
LKYYPTEKINSFFAQCLKLIVGNQANFKRIFDLEAEVKLQFSKETYEEDLESLLEKI
GDEYLDIFLQAKKVHDAILLSEIISSTVKHTKAKLSSGMVERYERHKADLAKFKQF
VKENVPQKSTAFFKDTTKNGYAGYIKGKTTQEEFYKFVKKELSGVVGSEPFLEKID
QETFLLKQRTYTNGVIPHQVHLIELKAIIDQQKQHYPFLEEAGPKIIALFKFRIPYYV
GPLAKEQEASSFAWIERKTAEKIHPWNFSEVVDIEKSAMRFIQRMTKQDTYLPTEK
VLPKNSLLYQKYMIFNELTKVSYKDERGVKQYFSGDEKQQIFKQLFQKERGKITV
KKLQNFLYTHYHIENAQIFGIEKAFNASYSTYHDFMKLAKTNQKAMQEWLEQPE
MEPIFEDIVKILTIFEDRQMIKHQLSKYQEVFGEKLLKEFARKHYTGWGRFSAKLIH
GIRDRKTNKTILDYLINDDDVPANRNRNLMQLINDEHLSFKEEIAKATVFSKHKSL
VDVIQDLPGSPAIKKGIWQSLKIVEELIAIIGYKPKNIVIEMARENQKTHRTKPRLKA
LENGLKQIGSTLLKEQPTDNKALQKERLYLYYLQNGRDMYTGEPLEIENLHQYEV
DHIIPRSFIVDNSIDNKVLVARKQNQKKRDDVPKKQIVNEQRIFWNQLKEAKLISPK
KYAYLTKIELTPEDKARFIQRQLVETRQITKHVANILHQSFHQEEEGTDCDGVQIIT
LKATLTSQFRQTFGLYKVREINPHHHAHDAYLNGFIANVLLKRYPKLAPEFVYGK
YVKYSLARENKATAKKEFYSNILKFFESDEPFCDENGEIYWEKSHHLPRIKKVLSS
HQVNVVKKVEQQKGGFYKETVNSKEKPDKLIERKNNWDVTKYGGFGSPVIAYAI
AFVYAKGKTQKKTRAIEGITIMEQAAFEKDPTTFLKEKGFPQVTEFIKLPKYTLFEF
DNGRRRFLASHKESQKGNPFILSDQLVTLLYHAQHYDKITYQESFDYVNTHLSDFS
AILTEVLAFAEKYTLADKNIERIQELYEENKYGEISMIAQSFLQLLQFNAIGAPADF
KFFGVTIPRKRYTSLTEIWDATIIYQSVTGLYETRIRMGDLWAGEQ (SEQ ID NO: 2)
Wild Type Streptococcus equinus strain AG46 (SeqCas9)
MTNGKILGLDIGVASVGVGIIEAKTGKVIHANSRLFSAANAENNAERRGFRGARRL
TRRKKHRVKRVRDLFEKYDISTDFRNLNLNPYELRVKGLSEQLTNEELFAALRTIA
KRRGISYLDDAEDDSTGSSDYAKSIDENRRLLKSMTPGQIQLERLEKYGQLRGNFT
VYDENGEAHRLINVFSTSDYKNEARKILETQSNYNKQITDEFIEDYIEILTQKRKYY
HGPGNEKSRTDYGRFRTDGTTLENIFGILIGKCSFYPEEYRASKASYTAQEFNFLND
LNNLKVPTETGKLSTEQKEYLVDFAKNSKTLGASKLLKEIAKLVDGDVKDISGYR
EDSKGKPDLHTFEPYRKLKFNLTTVDIDNLSRDILDKLANILTLNTEREGIEDAINR
NLPEQFTKEQISEIVQIRKSQSSAFNKGWHSFSAKLMNELIPELYATSEEQMTILTRL
EKFKATKKSSKNTKTIDEKEITDEIYNPVVAKSVRQTIKIINAAVKKYGDFDKIVIE
MPRDKNADDEKKFIDKKNKENKKEKDDSLKRAAYLYNGTDKLPDDVFHGNKQL
ATKIRLWYQQGERCLYSGKPILIQDLVHNSNNFEIDHILPLSLSFDDSLANKVLVYA
WTNQEKGQKTPYQVIDSMDAAWSFREMKDYVLKQKGIGKKKREYLLTTENIDKI
EVKKKFIERNLVDTRYASRVVLNSLQTALKELGKDTKVSVVRGQFTSQLRRKWKI
DKSRETYHHHAVDALIIAASSQLKLWQKHENLMFENYGENQVVNKETGEILSISD
DEYKELVFQPPYQGFVNTISSKAFEDEILFSYQVDSKFNRKVSDATIYSTRKAKLGK
DKKEETYVLGKIKDIYSQDGFDTFIKRYKKDKTQFLMYQKDPLTWENVIEVILRDY
PTTKKSEDGKNDVKCNPFEEYRRENGLICKYSKKGKGTPIKSLKYYDKKLGNHISI
TPKESKNDVVLQSLNPWRADLYFNPDTLKYELMGLKYSDLSFEKGTGKYHISQEK
YDEIKEKEGIGQNSEFKFTLYRNDLILIKDTESGEQEIYRFLSRTMPNVKHYVELKP
YDKEKFNGGQELIKSLGEADKVGRCLKGLSKPGISIYKVRTDVLGNKFFVKKEGD
KPKLDFKNNKK (SEQ ID NO: 3)
Wild Type Staphylococcus simulans strain 19 (SsiCas9)
MNNSYILGLDIGITSVGYGIIEYETRDVIDAGVRLFKEANVENNEGRRSKRGARRL
KRRRRHRLQRVKKMLFDYKLLNEDSEISGINPYEARVKGLSEKLSDEEFSAALLHL
AKRRGVHNVSDVEEDTGNELSTKEQIARNNKALEDKYVAELQLERLKEQGEVRG
AANRFKTSDYIKEAKQLLKTQSDYHKIDETFIETYISLLETRRTYYEGPGEGSPFGW
KDIKEWYEMLMGHCTYFPEELRSVKYAYNADLYNALNDLNNLVIARDENEKLEY
YEKFQIIENVFKQKKKPTLKQIAKEILVNEEDIKGYRVTSTGKPEFTNFKIYHDIKGI
TSRKEILENADLLNQIAEILTIYQSAEDIQEELAELEPKLTQEEIEQISNLTGYTGTHR
LSLKAINLILDELWNTSDNQMTIFNRLKLVPKKVDLSQQKEIPTSLVDDFILSPVVK
RSFIQSIKVINAIIKKFGLPKDIIIELAREKNSKEAQKFINEMQKRNRQTNERIEKIIKE
TGKEKAKFLIEKIKLHDMQEGKCLYSLESIPLEDLLNNPYHYEVDHIIPRSVSFDNSF
NNKVLVKQEENSKKGNRTPFQYLSSSDAKISYETFKKHILNLSKGKGRVSKKKKE
YLLEERDINRFSVQKDFINRNLVDTRYATRELMNLLRSYFRVNDLDVKVKSINGGF
TSFLRTKWKFKKERNQGYKHHAEDALVIANADFIFKEWKKLDTTNKVMENQTVE
EQQAENMPGIETDDEYKEIFVIPRQIQSIKDFKDYKYSHRVDKKPNRELVNDTLYST
RKDDKGNTLIINNIKGLYDKDNDKLKNLIKKSPEKLLMYHHDPQTYQKLKTIMEQ
YSNEKNPLYKYHEETGNYLTKYSKKDNGPIIKKVKYYGKKLNAHLDITNDYSNSQ
NKIVKLSLKPYRFDVYLDNGGYKFVTVKNLDVIKKEGFFKIDSNAYEKAKSEKKID
ENAVFIASFYNNDLIKIDGELYRIVGVNNDTRNVVELNMIPITYKEYLENINDKRTP
RILKTISQKTYSIEKYSTDILGNLYKVKSKKKPQMIMKG (SEQ ID NO: 4)
Wild Type Streptococcus intermedius B196 strain G1552 (SinCas9)
MNGLVLGLDIGIASVGVGILNKETGEIIHVNSRIFPAATADSNVERRGFRQGRRLGR
RKKHRSARLNDLFEEFGFITDFSAVPLNLNPYALRVKGLSEELTNEELFIALKNIIKR
RGISYLDDASEDGETASNEYGKAVEENRKLLADKTPGQIQLERFEKYGQVRGDFT
VVENGENHRLINVFSTSAYKKEAERILRRQQEFNVRISDEFIEAYLTILTGKRKYYH
GPGNEKSRTDYGRFRTDGTTLDNIFGILIGKCTFYPDEYRAAKASYTAQEFNLLND
LNNLTVPTETKKLRPEQKRQIVEYARTAKTLGTPTLLKYIAKLVDGSIDDIKGYRID
KSDKPEMHTFDAYRKMRTLELVDVDILSRETLDDLAYILTLNTESEGILEALNSKM
PGTFTKEQIDELIQFRKKNSAVFGKGWHNFSLKLMNELISELYETSEEQMTILTRLG
KQRSREISKRTKYIDEKELTDEIYNPVVAKSVRQAIKIINLATKKYGIFDNIVIEMAR
ESNEDDEKKAIQNVQKANEDEKKAAMEKAADLYNGKKELPDSIFHGHKELATKIR
LWHQQGERCLYTGKNISIHDLIHNPHQYEIDHILPLSLSFDDGLANKVLVLATANQ
EKGQRTPFQALDSMDDAWSYIEFKQYVRNSKSLSNKKKDYLLTEEDISKIEVKQKF
IERNLVDTRYSSRVVLNTLQEFYKTNDFDTKISVVRGQFTSQLRRKWKIEKSRDTY
HHHAVDALIIAASSQLRLWKKQNNPLISYREGQLVDPETGEILSLTDDEYKELVFRP
PYDYFVDTLKSKSFEDSILFSYQVDSKYNRKISDATIYGTRKAQLGKDKQEETYVL
GKIKDIYSQKGYEDFIKRYKKDTTQFLMYHKDPQTFAKVIEEILKTYPDKELNEKG
KEIPCNPFEKYRQENGPIRKYSKKGKGPEIKSLKYYDNKLGNHIDITPVNSQNQVVL
QSLKPWRTDVYFNPQTSKYELMGLKYSDLRFEKGSGSYGISPEKYNKVKAKEGVD
EDSEFKFTLYKNDLILIKDTETGEQQLFRYGSRNDTSKHYVELKPYEKAKFEGNQQ
LMNLLGTVAKGGQCLKGINKPNLSIYKVKTDVLGNKHFIKKEGDQPQLNFKKKI
(SEQ ID NO: 5)
Wild Type Streptococcus sanguinis SK330 (SsaCas9)
MENKNYSIGLDIGTNSVGWAVITDDYKVPSKKMKVFGNTDKHFIKKNLIGALLFD
EGATAEDRRLKRTARRRYTRRKNRLRYLQEIFSEEISKLDSSFFHRLDDSFLVPKDK
RGSKYPIFATLEEEKEYHKKFPTIYHLRKHLADSKEKTDLRLIYLALAHMIKYRGH
FLYEESFDIKNNDIQKIFNEFISIYDNTFEGSSLSGQNAQVEAIFTDKISKSAKRERVL
KLFSDEKSTSLFSEFLKLIVGNQADFKKHFDLEEKAPLQFSKDTYDEDLENLLGQIG
DGFTDLFLVAKKLYDAILLSGILTVTDPSTKAPLSASMIERYESHQKDLAALKQFIK
NNLPKRYNEVFSDQSKDGYAGYIDGKTTQEAFYKYIKNLLSKFEGADYFLDKIERE
DFLRKQRTFDNGSIPHQIHLQEMNAILRRQGEHYPFLKENREKIEKILTFRIPYYVGP
LARGNRDFAWLTRNSDQAIRPWNFEEIVDKASSAEEFINKMTNYDLYLPEEKVLP
KHSLLYETFAVYNELTKVKFIAEGLRDYQFLDSGQKKQIVNQLFKEKRKVTEKDII
HYLHNVDGYDGIELKGIEKQFNASLSTYHDLLKIIKDKEFMDDPKNEEILENIVHTL
TIFEDREMIKQRLAQYDSIFDEKVIKALTRRHYTGWGKLSAKLINGICDKQTGDTIL
DYLIDDGKINRNFMQLINDDGLSFKEIIQKAQVVGKTDDVKQVVQELPGSPAIKKG
ILQSIKIVDELVKVMGYAPESIVIEMARENQTTARGKKNSQQRYKRIEDSLKNLAP
GLDSNILKENPTDNIQLQNDRLFLYYLQNGKDMYTGKPLDIDQLSSYDIDHIIPQAF
IKDDSIDNRVLTSSKDNRGKSDNVPSLEVVQKRKAFWQQLLDSKLISERKFNNLTK
AERGGLDERDKVGFIRRQLVETRQITKHVAQILDASFNTEVNEKNQKIRTVKIITLK
SNLVSNFRKEFELYKVREINDYHHAHDAYLNAVVAKAILKKYPKLEPEFVYGDYQ
KYDLKRYISRFKPSKEIEKATEKYFFYSNLLNFFKEEVHYADGIIVKRENIEYSKDT
GEIAWNKEKDFATIKKVLSYPQVNIVKKTEIQTHGLDRGKPKGLFNSNPSPKPSEDS
KENLVPIKQGLDPRKYGGYAGISNSYAVLVKAIVEKGAKKQQKTILEFQGISILDKI
NFENNKENYLLKKRYIEILSTITLPKYSLFEFPDGTRRRLASILSTNNKRGEIHKGNE
LVLPGKYTTLLYHAKNINKKLEPEHLEYVEKHRNDFAKLLECVLNFNDKYVGALK
NGERIRQAFTDWETVDIEKLCFSFIGPENSKNAGLFELTSQGSASDFEFLGVKIPRYR
DYAPSSLLKATLIHQSITGLYETRIDLSKLGED (SEQ ID NO: 6)
Wild Type Streptococcus sp. C150 (Ssc2Cas9)
MSDLVLGLDIGIGSVGVGILNKVTGEIIHKNSRIFPAAQAENNLERRTNRQGRRLTR
RKKHRRVRLNHLFEESGLITDFTKVSINLNPYQLRVKGLTAELSNEELFIALKNMV
KHRGISYLDDASDDGNSSVGDYAQIVKENSKQLETKTPGQIQLERYQKYGQLRGD
FTVEEDGRKHRLINVFPTSAYHAEALRILQTQQEFNPQITDEFINSYLEILTGKRKYY
HGPGNEKSRTDYGKYTTKKDAQGQYITLNNIFGILIGKCTFYPEEYRAAKASYTAQ
EFNLLNDLNNLTVPTETKKLSEEQKYQIITYVKNEKAMGPAKLFKYIAKLLSCDVA
DIKGYRIDKSDKAEIHTFEAYRKMKTLETLDVKKMAREELDKLAYVLTLNTEREGI
QEALDHEFADGTFSQEQVDELVQFRKANSSIFGKGWHSFSVKLMMELIPELYATSE
EQMTILTRLGKQKTTSSSNKTKYIDEKQLTEEIYNPVVAKSVRQAIKIVNAAIKKYG
DFDNIVIEMARETNEDDEKKAIQKIQKANKAEKDAAMRKAANQYNGKAELPHSV
FHGHKQLATKIRLWHQQGERCLYTGKTISIHDLINNPNQFEIDHILPLSITFDDSLAN
KVLVYATANQEKGQRTPYQALDSMDDAWSFRELKAFVRESKALSNKKKEYLLTE
EDISKFDVRKKFIERNLVDTRYASRVVLNALQEHFRTHKIDTKVSVVRGQFTSQLR
RHWGIEKTRDTYHHHAVDALIIAASSQLNLWKKQKNTLVSYSEDQLLDIETGELIS
DDEYKESVFKAPYQHFVDTLKSKEFEDSILFSYQVDSKFNRKISDATIYATRKAKL
DKEKKEYTYTLGKIKDIYALGTKTPSKTGFYKFLDLYKKDKSQFLMYQKDRRTW
DEVIEKILEQYRPFKEKDKNGKEVDFNPFEKYRIENGPIRKYSRKGNGPEIKSLKYY
DNLLGRFVDITPSESKNPVALLSLNPWRTDVYYNTETRKYEFLGLKYADLCFEKG
GSYGISKVKYNKIREKEGIGKNSEFKFTLYKNDLILIKDTETNRQQIFRFWSRTGKD
NPKSFEKHKLELKPYEKTRFEKGEELKVLGKVPPSSNRLQKNMQIENLSIYKVRTD
VLGNQHIIKNEGDKPKLDF (SEQ ID NO: 7)
Wild Type Streptococcus oralis subsp. oralis strain RH_1735_08 (Sor2Cas9)
MNGLVLGLDIGIASVGVGILEKNSGKIIHANSRIFPAATADNNVERRKNRQARRLH
RRKKHRGVRLQDIFEDYGLLTDFSKVSINLNPYRLRVDGLDQQLTNEELFIALKNI
VKRRGISYLDDASEDGGTVSSDYGKAVEENRKLLAEKTPGQIQLERFEKYGQVRG
DFTVVENGEKRRLINVFSTSAYRKEAERILRKQQEFNSKITDEFIEDCLKILTGKRK
YYHGPGNEKSRTDYGRFRTDGTTLDNIFGILIGKCTFYPNEYRASKASHTAQEFNL
LNDLNNLTVPTETKKLSEEQKKVIVEYAKEAKTLGASTLLKYIAKMIDASVDQISG
YRVDVNNKPEMHTFEVYRKMQSLETISVGELSRNVLDELAHILTLNTEREGIEEAI
NTKLKDSFSQDQVLELVQFRKNNSSLFSRGWHNFSLKLMMELIPELYETSEEQMTI
LTRLGKQKSKETSKRTKYIDEKELTEEIYNPVVAKSVRQAIKIINEATKKYGIFDNIV
IEMARENNEEDAKKDYIKRQKANQDEKNAAMEKAAFQYNGKKELPDNIFHGHKE
LATKIRLWHQQGEKCLYTGKNIPISDLIHNQYKYEIDHILPLSLSFDDSLSNKVLVL
ATANQEKGQRTPFQALDSMDDGWSYREFKSYVKESKLLGNKKKEYLLTEEDISKI
EVKQKFIERNLVDTRYSSRVVLNALQDFYKAHKFDTTISVVRGQFTSQLRRKWGL
EKSRETYHHHAVDALIIAASSQLRLWKKQNNPLIAYKEGQFVDSETGEILSLSDDE
YKELVFKAPYDHFVDTLRSKTFEDSILFSYQVDSKYNRKISDATIYATRKAKLDKD
KSEETYVLGKIKDIYSQAGYDAFIKIYNKDKSKFLMYHKDPQTFEKVIEEILRTYPS
KELNDKNKEIPCNPFEKYRQENGPIRKYSKKGNGPEIKCLKYYDNKLGNYIDITPD
GSDNQVVLQSIAPWRTDVYYNHKTGKYEFLGLKYSDLYFEKGTGKYKISKEKYD
NIKKIEGVVETSEFKFTLYKNDLILIKDVEKGQEQLFRFLSRNNKGKHQVQLKPMN
KSDFEKGEKLIDIFGTVPNSTTQCVKGLNKSNISIFKVKTDVLGKKHIIKKEGDEPKL
KF (SEQ ID NO: 8)
Wild Type Streptococcus oralis SK313 (SorCas9)
MNGLVLGLDIGIASVGVGILEKNSGKIVHASSRIFPAATADNNVERRKNRQARRLH
RRKKHRGARLKDLFEYYGLLTDFSKVSINLNPYRLRVDGLDQQLTNEELFIALKNI
VKRRGISYLDDASEDGGTVSSDYGKAVEENRKLLAEQTPGQIQLERFEKYGQLRG
DFTVVENSEKCRLINVFSTSAYKKEAERILRKQQEFNNQITDEFIEDYLKILTGKRK
YYHGPGNEKSRTDYGRFRTDGATLDNIFGILIGKCTFYPNEYRASKASYTAQEFNL
LNDLNNLTVPTETKKLSEEQKKTIIEYAKSAKTLGASTLLKYIAKMIDASVDQIRGY
RVDVNNKPEMHTFEVYRKMQSLETISVGELSRNILDELAHILTLNTEREGIEEAINT
KLRDSFSQDQVLELVQFRKNNSSLFSKGWHNFSLKLMMELIPELYETSEEQMTILT
RLGKQKSKETSKRTKYIDEKEVTEEIYNPVVAKSVRQAIKIINEATKKHGIFDNIVIE
MARENNEEDAKKDYIKRQKANQDEKYAAMEKAAFQYNGKKELPDNIFHGHKEL
ATKIRLWHQQGEKCLYTGKSIPISDLIHNQYKYEIDHILPLSLSFDDSLSNKVLVLAT
ANQEKGQRTPFQALDSMDDAWSYREFKSYVKESKLLGNKKKEYLLTEEDISKIEV
KQKFIERNLVDTRYSSRVVLNALQDFYKNHNFDTTISVVRGQFTSQLRRKWGLEK
SRETYHHHAVDALIIAASSQLRLWKKQNNPLIAYKEGQFVDSQTGEIISLTDDEYK
ELVFKAPYDHFVDTLSSKTFEDSILFSYQVDSKFNRKISDATIYATRKAKLDKEKKE
YTYTLGKIKDIYSLGTKTPSKTGFYKFLDLYNKDKSQFLMFQKDRKTWDEVIEKIM
EQYRPFKEYDKAGKLVDFNPFEKYRQENGPIRKYSKKGNGPEIKSLKYYDILLGKH
KNITPEGSRNTVALLSLNPWRTDVYYNMETKKYEFLGLKYADLPFEEGGAYGIST
ETYNELREKEGIGKNSEFKFTLYKNDLILIKDTETNCQQFFRFWSRTGKDNPKSFEK
HKIELKPYEKAKFEKGEELEVLGKVPPSSNQFQKNMQIENLSIYKVKTDVLGNKHF
IKKEGDKPKLKF (SEQ ID NO: 9)
Wild Type Staphylococcus warneri strain 691 (SwaCas9)
MKEKYILGLDLGITSVGYGIINFETKKIIDAGVRLFPEANVDNNEGRRSKRGSRRLK
RRRIHRLDRVKSLLTEYNLINREQIPTSNNPYQIRVKGLSEILSKDELAIALLHLAKR
RGIHNINVSSEDEDASNELSTKEQINRNNKLLKNKYVCEVQLQRLKEGQIRGEKNR
FKTTDILKEIDQLLKVQKDYHNLDIDFINQYKEIVETRREYFEGPGQGSPFGWNGD
LKKWYEMLMGHCTYFPQELRSVKYAYSADLFNALNDLNNLIIQRNDSEKLEYHE
KYHIIENVFKQKKKPTLKQIAKEIGVNPEDIKGYRITKSGTPQFTEFKLYHDLKSIVF
DKSILENEAILDQIAEILTIYQDEESIKEELNKLPEILNEQDKAEIAKLTGYNVTHRLS
LKCIHLINEELWQTSRNQMEIFNYLNIKPNKVDLSEQNKIPKDLVDEFILSPVVKRT
FIQSINVINKVIEKYGIPEDIIIELARENNSDDRKKFINNLQKKNEATRKRINEIIGQTG
NQNGKRIVEKIRLHDQQEGKCLYSLESIPLMDLLNNPQNYEVDHIIPRSVAFDNSIH
NKVLVKQIENSKKGNRTPYQYLNSSDANLSYNQFKQHILNLSKSKDRISKKKKDY
LLEERDINKFEVQKEFINRNLVDTRYATRELTSYLKAYFSANNMDVKVKTINGSFT
NHLRKVWRFDKYRNHSYKHHAEDALIIANADFLFKENKKLQNANKILEKPTIEND
TQKVTVEKEEDYNNMFETPKLVEDIKQYRDYKFSHRVDKKPNRQLIKDTLYSTRM
KDEHNYIVQTITDIYGKDNTNLKKQFNKNPEKFLMYQNDPKTFEKLSIIMKQYSDE
KNPLAKYYEETGEYLTKYSKKNNGPIVKKIKLLGNKVGNHLDVTNKYENSTKKL
VKLSIKNYRFDVYLTEKGYKFVTIAYLNVFKKDNYYYIPKDLYQELKAKKKIKDT
DQFIASFYKNDLIKLNGDLYKIIGVNSDDRNIIELDYYDIKYKDYCEINNIKGEPRIK
KTIGKKTESIEKLTTDVLGNLYLHTTEKAPQLIFKRGL (SEQ ID NO: 10)
Wild Type Staphylococcus sciuri strain SNUC_2430 (SscCas9)
MKRNYILGLDIGITSVGYGIIDYETRDVIDAGVRLFKEANVENNEGRRSKRGARRL
KRRRRHRLQRVKKLLFDYNLLTDHSELSGINPYEARVKGLSQKLSEVEFSAALLHL
AKRRGVHNVNEVEEDTGNELSTKEQISRNSKALEEKYVAELQLERLKTDGEVRGP
NNRFKTSDYVKEAKQLLKVQKAYHQLDQSFIDTYIDLLETRRTYYEGPGEGSPFG
WKDIKEWYEMLMGHCTYFPEELRSVKYAYNADLYNALNDLNNLVIARDENEKLE
YYEKFQIIENVFKQKKKPTLKQIAKELLVNEEDIKGYRVTSIGKPEFTNFKIYHDIKG
ITERKEVLENAELLDQIAEILTIYQSSEDVQEELANLNSELTQEEIEQISNLKGYTGT
HNLSLKAINLILDELWHTNDNQMAIFNRLKLVPKKVDLSQQKEIPTTLVDDFILSPV
VKRSFIQSIKVINAIIKKYGLPNDIIIELAREKNSKDAQKMINEMQKRNRQMNERIEE
IIRTTGKENAKYLIEKIKLHDMQEGKCLYSLESIPLEDLLNNPFNYEVDHIIPRSVSF
DNSFNNKVLVKQEENSKKGNRTPFQYLSSSDSKISYETFKKHILNLAKGKGRISKT
KKEYLLEERDINRFSVQKDFINRNLVDTRYATRELMNLLRSYFRVNNLDVKVKSIN
GGFTSFLRRKWKFKKERNKGYKHHAEDALIIANADFIFKEWKKLDKAKKVMENQ
TVEEKQAESMPEIETEQEYKEIFITPHQIQHIKGFKDYKYSHRVDKKPNRELINDTL
YSTRKDDKGNTLIVNNLNGLYDKDNDKLKKLMNKSPEKLLMYHHDPQTYQKLK
LIMEQYGDEKNPLYKYYEETGNYLTKYSKKDNGPVIKKIKYYGKNLKAHLDITDD
YPNSRNKVVKLSVKPYRFDVYLDNDIYKFVTVKNLDVIKKEDYYEVNSKCYKEA
KKLKKISDQAEFIASFYNNDLIKINGELYRVIGVNNDLLNRIEVNMINITYREYLEN
MNDKRPPRIIKTIASKTQSIKKYSTDILGNLYEVKSKQKPQMIMKG (SEQ ID NO:
11)
Wild Type Streptococcus gallolyticus strain AM24-4 (SgaCas9)
MTNGKILGLDIGIASVGVGIIEAKTGKVVHANSRLFSAANAENNTERRGFRGSRRL
NRRKKHRVKRVRDLFEKHEIVTDFRNLNLSPYELRVKGLTEQLTNEELFAALRTIS
KRRGISYLDDAEDDSTGSTDYAKSIDENRRLLKNKTPGQIQLERLEKYGQLRGNFT
VYDENGEAHRLINVESTSDYEKEARKILETQADYNKKITAEFIDDYVEILTQKRKY
YHGPGNEKSRTDYGRFRTDGTTLENIFGILIGKCNFYPDEYRASKASYTAQEYNFL
NDLNNLKVPTETGKLPTEQKESLVEFAKNTATLGPSKLLKEIAKILDCKVDEIKGY
REDDKGKPDLHTFEPYRKLKFNLESINIDDLSREVIDKLADILTLNTEREGIEDAIKR
NLPNQFTEEQISEIIKVRKSQSTAFNKGWHSFSAKLMNELIPELYATSDEQMTILTRL
EKFKVNKKSSKNTKTIDEKEVTDEIYNPVVAKSVRQTIKIINAAVKKYGDFDKIVIE
MPRDKNADDEKKFIDKRNKENKKEKDDALKRAAYLYNSSDKLPDEVFHGNKQLE
TKIRLWYQQGERCLYSGKPIPIHDLVHNSNNFEIDHILPLSLSFDDSLANKVLVYDW
ANQEKGQKTPYQVIDSMDAAWSFREMKDYVLKQKGLGKKKRDYLLTTENIDKIE
VKKKFIERNLIDTRYASRVVLNSLQSALRELCKDTKVSVIRGQFTSQLRRKWKIDK
SRETYHHHAVDALIIAASSQLKLWEKQDNLMFIDYGNNQVVDKETGEILSVSDDE
YKELVFQPPYQGFVNTISSKGFEDEILFSYQVDSKYNRKVSDATIYSTRKAKIGKDK
KEETYVLGKIKDIYSQNGFDTFIKKYNKDKTQFLMYQKDPLTWENVIEVILRDYPT
TKKSEDGKNDVKCNPFEEYRRENGLICKYSKKGKGTPIKSLKYYDKKLGNCIDITP
EESRNKVILQSINPWRADVYFNPETLKYELMGLKYSDLSFEKGTGKYHISQEKYDA
IKEKEGIGKKSEFKFTLYRNDLILIKDTASGEQEIYRFLSRTMPNVNHYAELKPYDK
EKFDGGQELMEVFGKVANGGQCLKSLNKSNISIYKVRTDVLGNKYFVKKEGDKP
KLNFKNNKK (SEQ ID NO: 12)
Wild Type Lactobacillus kullabergensis strain Biut2 (LkuCas9)
MKRVNEDYILGLDIGTNSCGWAVTDKKNNLLKLRGKTAIGSHLFEEGHTAADRR
GFRTTRRRLKRRKWRLRLLEEIFAEPMAKVDPGFFVRLHQSWVSPLDKDRKKYNA
IVFPTAKEDQAFYKHYATIYHLRDELMTQDRQFDLREIFLAIHHIVKYRGNFLQDTP
VKDFEASKIEVGPILSHINNAFAEKIVEDQDPIELNVANAADIEDVIRGKDAEKTVY
KLDKVKKIAKLLTDSTAKEEKNVAKQIANAIMGYKTQFETILDKEIDKSDKAQWE
FKLSDADADDKLDALLPDLDETDQTVVAEIEKLFSAITLSTIVDENKSLSQSMVEK
YKKHKKDYKKLKKYINTLQDQTKAKKLLLAYDLYVNNRHGRLLEAKKTFEDKK
KKKALTKDEFYKIVKDNLDDSDLAHEIQQEIAADNFMPKQRTNSNGVIPFQLHQIE
LDKIIANQGKYYPFLAAENPVEDHRKQAPYKLDELVRFRVPYYVGPMITADEQEK
TSGKSFAWMVRKEDGQITPWNFEQKVDRQESANKFIKRMTIKDTYLLSEDVLPAN
SLLYQRFEVLNELNNIRINGSRISVDLKQQIFNDLFEEKKTVTEKSLTSYLKQNLHLP
TVEIKGLADPTKFNSSLASYYHLKSLHVFDKELADPQYQKDFEKIIEYSSIFEDKKIF
QDKLHAEFKWLTPEQFKAISTWRLQGWGRLSRKLLVELHDTNGQNIMEQLWDSQ
KNFMQIVTEPDFKDAIAKENQNVTRANGVEEILADAYTSPANKKAIRQVVKVVAD
VVKAAGGKKPAQFAIEFTRDPDKNPQLSHIRGTKLLKAYQETAGELVDQKLTDSL
KEAMTSRKLLKDKYFLYFMQAGRDAYTGQKINIDEVSTNYQIDHILPQSFIKDDSF
DNRVLTATPLNAEKSDDVPYKRFANNYVSDMKMTVGEMWKHWQKAGIINKHKL
GNLLLDPDRLNKFQKSGFINRQLVETSQIIKLVSVILQNKYPDAEIITVKAGDNSAL
RQRLNLYKSRDVNDYHHAIDAYLSIICGNFLYQVYPKYRPYFVYGKYKKFSQDPD
LQKEVIKHFKGFTFMWPLLQKDNSERKAPEKIKENNSDRIVFYKHPDIFDKLRKAY
NYKYMLVSRETTTENSGLFDVTIYPRGERDLAKTRKLIPKSNGLDPKIYGGYSGNT
DAYMVIVKIDKGKESIYKVIGVPMRALASLNRAKKQGNYKEELHQVLEPQIMFDK
NGKPKRSVKGFRIIKDHVPFKQVVLDGDKKFMLNSSTYEINAKQLTLTPETMRIVT
DNLKKGEDQDQLLVKAYDEILQKVDQYLPLFDVNKFRNSLHLGRAKFLDLAVND
KKITLTNILNGLHDNLVTPDLKNIGIKTPLGKLQVPSGIVLSSEAILIFQSPTGLFEKR
VRIADL (SEQ ID NO: 13)
Wild Type Streptococcus suis strain LSS83 (SsuCas9)
MSNGKILGLDIGIASVGVGVIDAQTGEIIHASSRIFPSANAANNAERRTFRGSRRLIR
RKKHRIKRLDDLFNDFHINLDGEMSTDNPYVLRVKGLSQKLTVEELYISIKNIMKR
RGISYLDDAESDNEAGRSDYAKAIERNRQLLTSKTPGEIQLERLEKYGQLRGNFTII
DEEGQSQQIINVFSTSDYVKEVEKILDCQKMYHKFISDEFCDKLIELLREKRKYYVG
PGNEKSRTDYGIYRTDGTTLENLFGILIGKCTFYPDQYRSSRASYTAQEFNFLNDLN
NLTVPTETKKLSQEQKEFLVNYAKETSVLGAGKILQQIAKLADCKVEDIRGYRLDN
KDKPELHTFETYRAMKGLVPLVDIGVLSREQLDILADILTLNTDFEGIREALKKQLP
NVFDEKQVKGLASFRKSKSQLFAKGWHNLSQKIMLEVIPELYATSDEQMTILTRLG
KFEKSSVAEYPSSINVDEITDEIYNPVVAKSIRQTIKIINASIKKWDEFDQIVIEMPRD
RNEDEEKKRIADGQKANAKEKADSILRAAELYCAGKVLPDYVYNGHNQLATKIR
LWYQQGERCIYTGQPISIHDLIHNQNQYEIDHILPLSLTFDDSLSNKVLVLATANQE
KAQRTPYNYLKSATSAWSYREFKDYVTKRKGIGKKKCEYLTFEEDINGFEVRSKFI
QRNLVDTRYASKVILNALQDYFKISGIQTKVSVVRGQFTSQLRHKWGIEKTRETYH
HHAVDALIIAASSQLRLWKKQESPLVVDYQEGRQVDLETGEILELTDEQYKELVY
QPPYQGFVNTISSSAFDNEILFSYQVDSKVNRKISDATIYATRNAQLGKDKTEGIYV
LGKIKDIYTQAGYEAFLKRYTKDKTSFLMYHKDLDTWEKVIEIILRDYREYDEKGK
EIGNPFERYRRENGYVKKYSRKGNGTAIKSLKYYDNKLGNHIDITPENSRNAVVLQ
SLKPWRTDVYFNKETGKYEFLGIKYSDLSFEKGTGEYGISQEKYDSIKIAEGVAKK
SIFKFTLYKQDLLFIKDIENNFGKLLRFTSKNDTSKHYVELKPYDKNKFGTEEPLLP
VLGNVAKSGQCIKGLNKSNISIYKVRTDILGYRHFIKQEGEHPQLKFKK (SEQ ID
NO: 14)

In some embodiments, the Cas9 protein comprises one or more mutations in reference to SEQ ID NO: 1, 2, 3, 4, 5, 6, 7, 8, 9, 10, 11, 12, 13 or 14. For example, the amino acid sequence of the Cas9 protein comprises at least one, at least two, at least three, at least four, at least five, at least six, at least seven, at least eight, at least nine, at least 10 mutations in SEQ ID NO: 1, 2, 3, 4, 5, 6, 7, 8, 9, 10, 11, 12, 13 or 14. Various mutations are known in the art, and include for example, amino acid substitutions.

In some embodiments, two or more catalytic domains of Cas9 (RuvCI, RuvCII, RuvCIII) are mutated to produce an inactive, or “dead” Cas9 (dCas9) that lacks nucleic acid cleavage activity. In some embodiments, the one or more mutations are in the PAM Interacting, HNH, and or the RuvC domains. In some embodiments, Cas9 is mutated to reduce DNA cleavage activity to less than about 25%, 15%, 10%, 5%, 1%, 0.1%, 0.01% or lower with respect to its non-mutated form.

In some embodiments a nickase-mutant version of Cas9 is provided. In some embodiments, the nickase mutant has one or more amino acid substitutions in the RuvC and/or the HNH domains. Various nickase mutations are known with respect to SpCas9 (Streptococcus pyogenes) and include for example mutations at one or more of amino acid positions 10, 12, 17, 762, 840, 854, 863, 982, 983, 984, 986, 987 of wild type SpCas9. For example, an aspartic acid-to-alanine substitution that corresponds to D10A in SpCas9 results in the creation of a nickase. In some embodiments, the Cas9 described herein has one or more mutations that result in the creation of a nickase. In some embodiments, the Cas9 described herein has one or more mutations at an amino acid position that corresponds to one or more of amino acids 10, 12, 17, 762, 840, 854, 863, 982, 983, 984, 986, 987 of SpCas9.

In some embodiments, the mutation is an aspartic acid-to-alanine substitution (D10A) in the RuvC domain of Seq2Cas9. In some embodiments, the mutation is an aspartic acid-to-alanine substitution (D10A) in the RuvC domain of EhiCas9. In some embodiments, the mutation is an aspartic acid-to-alanine substitution (D10A) in the RuvC domain of SeqCas9 (e.g., corresponding to D10A in SpCas9). In some embodiments, the mutation is an aspartic acid-to-alanine substitution (D10A) in the RuvC domain of SsiCas9. In some embodiments, the mutation is an aspartic acid-to-alanine substitution (D9A) in the RuvC domain of SinCas9. In some embodiments, the mutation is an aspartic acid-to-alanine substitution (D11A) in the RuvC domain of SsaCas9. In some embodiments, the mutation is an aspartic acid-to-alanine substitution (D9A) in the RuvC domain of Ssc2Cas9. In some embodiments, the mutation is an aspartic acid-to-alanine substitution (D9A) in the RuvC domain of Sor2Cas9. In some embodiments, the mutation is an aspartic acid-to-alanine substitution (D9A) in the RuvC domain of SorCas9. In some embodiments, the mutation is an aspartic acid-to-alanine substitution (D10A) in the RuvC domain of SwaCas9. In some embodiments, the mutation is an aspartic acid-to-alanine substitution (D10A) in the RuvC domain of SscCas9. In some embodiments, the mutation is an aspartic acid-to-alanine substitution (D10A) in the RuvC domain of SgaCas9. In some embodiments, the mutation is an aspartic acid-to-alanine substitution (D13A) in the RuvC domain of LkuCas9. In some embodiments, the mutation is an aspartic acid-to-alanine substitution (D10A) in the RuvC domain of SsuCas9.

In some embodiments, the mutation is an aspartic acid-to-glycine substitution (D10G) in the RuvC domain of Seq2Cas9. In some embodiments, the mutation is an aspartic acid-to glycine substitution (D10G) in the RuvC domain of EhiCas9. In some embodiments, the mutation is an aspartic acid-to-glycine substitution (D10G) in the RuvC domain of SeqCas9 (e.g., corresponding to D10G in SpCas9). In some embodiments, the mutation is an aspartic acid-to-glycine substitution (D10G) in the RuvC domain of SsiCas9. In some embodiments, the mutation is an aspartic acid-to-glycine substitution (D9G) in the RuvC domain of SinCas9. In some embodiments, the mutation is an aspartic acid-to-glycine substitution (D11G) in the RuvC domain of SsaCas9. In some embodiments, the mutation is an aspartic acid-to-glycine substitution (D9G) in the RuvC domain of Ssc2Cas9. In some embodiments, the mutation is an aspartic acid-to-glycine substitution (D9G) in the RuvC domain of Sor2Cas9. In some embodiments, the mutation is an aspartic acid-to-glycine substitution (D9G) in the RuvC domain of SorCas9. In some embodiments, the mutation is an aspartic acid-to-glycine substitution (D10G) in the RuvC domain of SwaCas9. In some embodiments, the mutation is an aspartic acid-to-glycine substitution (D10G) in the RuvC domain of SscCas9. In some embodiments, the mutation is an aspartic acid-to-glycine substitution (D10G) in the RuvC domain of SgaCas9. In some embodiments, the mutation is an aspartic acid-to-glycine substitution (D13G) in the RuvC domain of LkuCas9. In some embodiments, the mutation is an aspartic acid-to-glycine substitution (D10G) in the RuvC domain of SsuCas9.

In some embodiments, such one or more mutations described herein converts Cas9 to an inactive, or “dead” version of Cas9 (dCas9). Accordingly, in some embodiments, the Cas9 protein comprises one or more mutations that inhibits the ability of Cas9 to cleave both strands of a DNA duplex.

In some embodiments, when coexpressed with a guide RNA, dead Cas9 generates a DNA recognition complex that can specifically interfere with transcriptional elongation, RNA polymerase binding, or transcription factor binding. In some embodiments, dead Cas9 is used to specifically target effector proteins of various functions to specific nucleic acid target sites.

In some embodiments, the engineered non-naturally occurring Cas9 is codon-optimized for human cells. The engineered, non-naturally occurring Cas9 is at least 80% (e.g., 80%, 81%, 82%, 83%, 84%, 85%, 86%, 87%, 88%, 89%, 90%, 91%, 92%, 93%, 94%, 95%, 96%, 97%, 98%, 99% or more) identical to SEQ ID NO. 1, 2, 3, 4, 5, 6, 7, 8, 9, 10, 11, 12, 13 or 14.

Exemplary Cas9 amino acid sequences with Nuclear Localization Signal (NLS) and a linker is provided in Table 2 below.

TABLE 2
Exemplary Cas9 Sequence with NLS and Linker

Streptococcus equinus ATCC 33317 Cas9 (Seq2Cas9) with Nuclear Localization Signal
(NLS) and Linker
MPKKKRKVGTMEKSYSIGLAIGTNSVGWSVITDDYKVPAKKMRVLGNTDKKYIK
KNLLGALLFDSGETAEATRLKRTARRRYTRRKNRLRYLQEIFAKEMAKVDESFFQ
RLDESFLTDDDKTFDSHPIFGNKAEEDAYHQKFPTIYHLRKHLADSQEKADLRLIY
LALAHMIKFRGHFLYDNFNDDNFDWRNIDIQKRYEEFIETYDSTLGESYLADISVD
AASILEEKVSKTERLENLLKYYPTEKKTTFFGNLIKLILGQQAKFKAIFNLEDEISLQ
FATPTYDEDLEELLGKIDNGDSYSELFVAAQNLYNTILLASFLKTDNKSAKAPLSTS
MIERYENHKKDLAKLKDFVKKNCPDQYHDIFRDKSKNGYAGYIDNGVSQNDFYT
FIGKCLEESLKKDKGAQYFLDKIDRDDFLRKQRTFDNGAFPYQIHLQEMHAILRRQ
GDYYPFLKENQDKIEKILTFRIPYYVGPLARKDSRFAWANYRSDEAITPWNFDEVV
DKEKSAEKFITRMTLNDLYLPEEKVLPKHSLIYETFTVYNELTNIKYVNDQGNAIHF
DSELKEKIFNQLFKENRKVSKKTLIDFLNNSEGIYTDKLVGIDEEVKYLNASLGTYH
DLKKILESFMDDEINEKIIEDIIQTLTLFEDIEMKRQRLQKYDDIFTPKQLKELARRN
YTGWGRLSYKLINGIRNKENNKTILDYLKNGNRNFMQLINDDRLSFKQIIIDARKIE
KLDNIESVVYNLPGSPAIKKGILQSIKIVDELVKVMGHNPDNIVIEMARENQTTNQG
KNRSQQRLKRLQDSMSNFKDSSISLKDVDNSDLQNDRLFLYYIQNGKDMYTGEEL
DIDHLSDYDIDHIIPQSFIKDNSIDNRVLTSSAKNRGKSDDVPGRDVVLKMKPFWK
KLYDVKLISKRKFDNLTKSEHGGLTESDKAGFIKRQLVETRQITKYVAQILDGREN
TKRDDNNKVIRDVKVITLKSSLVSQFRKDFGFYKVREINDYHHAHDAYLNAVVGT
AILKKYPKLAPEFVYGEYKKCDVRKLIAKSGDKSEIGKATAKYFFYSNLMNFFKR
VIRYSNGMIVVRPVIEYSKDTGEIAWDKEKDFKTVCKVLSCPQVNIVKKVEKQSH
GLDRGKPKGFYNANPSPKPKKGSKVNLVPIKANLNPKNYGGYAGISNSYAVLVDA
TIEKGAKKKLTRIQEFQGISIIDREKYEKNKVEFLKGLGYKEIYSIITLPKYSLFELAD
GSRRMLASILSTNNKRGEIHKGNELVLPAKYIPLLYHANRIHNTFETGHREYVEKHI
AEFKEIAEIILEFNNKYVNAKKNSSIIEKALESFDSFSLDEICDSFVGKLKKNNTKKN
SGLFELVSLGSASDFEFLETKVPRYRDYTPSSLLNATLIHQSITGLYETRIDLSKLGE
EKRPAATKKAGQAKKKKGSYPYDVPDYAYPYDVPDYAYPYDVPDYA (SEQ ID NO:
15)(D10A mutation, bold underlined italics)
Enterococcus hirae strain F1129E Cas9 (EhiCas9) with Nuclear Localization Signal
(NLS) and Linker
MPKKKRKVGTMTKDYTIGLAIGTNSVGWAVLTDDYQLMKRKMSVHGNTEKKKI
KKNFWGARLFDEGQTAEFRRTKRTNRRRLARRKYRLSKLQDLFAEELCKQDDCFF
VRLEESFLVPEEKQYKPASIFPTLEEEKEYYQKYPTIYHLRQKLVDSTEKEDLRLVY
LALAHLLKYRGHELFEGDLDTENTSIEESFRVFLEQYSKQSDQPLIVHQPVLTILTD
KLSKTKKVEEILKYYPTEKINSFFAQCLKLIVGNQANFKRIFDLEAEVKLQFSKETY
EEDLESLLEKIGDEYLDIFLQAKKVHDAILLSEIISSTVKHTKAKLSSGMVERYERH
KADLAKFKQFVKENVPQKSTAFFKDTTKNGYAGYIKGKTTQEEFYKFVKKELSGV
VGSEPFLEKIDQETFLLKQRTYTNGVIPHQVHLIELKAIIDQQKQHYPFLEEAGPKII
ALFKFRIPYYVGPLAKEQEASSFAWIERKTAEKIHPWNFSEVVDIEKSAMRFIQRMT
KQDTYLPTEKVLPKNSLLYQKYMIFNELTKVSYKDERGVKQYFSGDEKQQIFKQL
FQKERGKITVKKLONFLYTHYHIENAQIFGIEKAFNASYSTYHDFMKLAKTNQKA
MQEWLEQPEMEPIFEDIVKILTIFEDRQMIKHQLSKYQEVFGEKLLKEFARKHYTG
WGRFSAKLIHGIRDRKTNKTILDYLINDDDVPANRNRNLMQLINDEHLSFKEEIAK
ATVFSKHKSLVDVIQDLPGSPAIKKGIWQSLKIVEELIAIIGYKPKNIVIEMARENQK
THRTKPRLKALENGLKQIGSTLLKEQPTDNKALQKERLYLYYLQNGRDMYTGEPL
EIENLHQYEVDHIIPRSFIVDNSIDNKVLVARKQNQKKRDDVPKKQIVNEQRIFWN
QLKEAKLISPKKYAYLTKIELTPEDKARFIQRQLVETRQITKHVANILHQSFHQEEE
GTDCDGVQIITLKATLTSQFRQTFGLYKVREINPHHHAHDAYLNGFIANVLLKRYP
KLAPEFVYGKYVKYSLARENKATAKKEFYSNILKFFESDEPFCDENGEIYWEKSHH
LPRIKKVLSSHQVNVVKKVEQQKGGFYKETVNSKEKPDKLIERKNNWDVTKYGG
FGSPVIAYAIAFVYAKGKTQKKTRAIEGITIMEQAAFEKDPTTFLKEKGFPQVTEFIK
LPKYTLFEFDNGRRRFLASHKESQKGNPFILSDQLVTLLYHAQHYDKITYQESFDY
VNTHLSDFSAILTEVLAFAEKYTLADKNIERIQELYEENKYGEISMIAQSFLQLLQF
NAIGAPADFKFFGVTIPRKRYTSLTEIWDATIIYQSVTGLYETRIRMGDLWAGEQK
RPAATKKAGQAKKKKGSYPYDVPDYAYPYDVPDYAYPYDVPDYA (SEQ ID NO:
16)(D10A mutation, bold underlined italics)
Streptococcus equinus strain AG46 (SeqCas9) with Nuclear Localization Signal (NLS)
and Linker
MPKKKRKVGTMTNGKILGLAIGVASVGVGIIEAKTGKVIHANSRLFSAANAENNA
ERRGFRGARRLTRRKKHRVKRVRDLFEKYDISTDFRNLNLNPYELRVKGLSEQLT
NEELFAALRTIAKRRGISYLDDAEDDSTGSSDYAKSIDENRRLLKSMTPGQIQLERL
EKYGQLRGNFTVYDENGEAHRLINVFSTSDYKNEARKILETQSNYNKQITDEFIED
YIEILTQKRKYYHGPGNEKSRTDYGRFRTDGTTLENIFGILIGKCSFYPEEYRASKAS
YTAQEFNFLNDLNNLKVPTETGKLSTEQKEYLVDFAKNSKTLGASKLLKEIAKLV
DGDVKDISGYREDSKGKPDLHTFEPYRKLKFNLTTVDIDNLSRDILDKLANILTLNT
EREGIEDAINRNLPEQFTKEQISEIVQIRKSQSSAFNKGWHSFSAKLMNELIPELYAT
SEEQMTILTRLEKFKATKKSSKNTKTIDEKEITDEIYNPVVAKSVRQTIKIINAAVKK
YGDFDKIVIEMPRDKNADDEKKFIDKKNKENKKEKDDSLKRAAYLYNGTDKLPD
DVFHGNKQLATKIRLWYQQGERCLYSGKPILIQDLVHNSNNFEIDHILPLSLSFDDS
LANKVLVYAWTNQEKGQKTPYQVIDSMDAAWSFREMKDYVLKQKGIGKKKREY
LLTTENIDKIEVKKKFIERNLVDTRYASRVVLNSLQTALKELGKDTKVSVVRGQFT
SQLRRKWKIDKSRETYHHHAVDALIIAASSQLKLWQKHENLMFENYGENQVVNK
ETGEILSISDDEYKELVFQPPYQGFVNTISSKAFEDEILFSYQVDSKFNRKVSDATIY
STRKAKLGKDKKEETYVLGKIKDIYSQDGFDTFIKRYKKDKTQFLMYQKDPLTWE
NVIEVILRDYPTTKKSEDGKNDVKCNPFEEYRRENGLICKYSKKGKGTPIKSLKYY
DKKLGNHISITPKESKNDVVLQSLNPWRADLYFNPDTLKYELMGLKYSDLSFEKG
TGKYHISQEKYDEIKEKEGIGQNSEFKFTLYRNDLILIKDTESGEQEIYRFLSRTMPN
VKHYVELKPYDKEKFNGGQELIKSLGEADKVGRCLKGLSKPGISIYKVRTDVLGN
KFFVKKEGDKPKLDFKNNKKKRPAATKKAGQAKKKKGSYPYDVPDYAYPYDVPD
YAYPYDVPDYA(SEQ ID NO: 17) (D10A mutation, bold underlined italics)
Staphylococcus simulans strain 19 (SsiCas9) with Nuclear Localization Signal (NLS)
and Linker
MPKKKRKVGTMNNSYILGLAIGITSVGYGIIEYETRDVIDAGVRLFKEANVENNEG
RRSKRGARRLKRRRRHRLQRVKKMLFDYKLLNEDSEISGINPYEARVKGLSEKLS
DEEFSAALLHLAKRRGVHNVSDVEEDTGNELSTKEQIARNNKALEDKYVAELQLE
RLKEQGEVRGAANRFKTSDYIKEAKQLLKTQSDYHKIDETFIETYISLLETRRTYYE
GPGEGSPFGWKDIKEWYEMLMGHCTYFPEELRSVKYAYNADLYNALNDLNNLVI
ARDENEKLEYYEKFQIIENVFKQKKKPTLKQIAKEILVNEEDIKGYRVTSTGKPEFT
NFKIYHDIKGITSRKEILENADLLNQIAEILTIYQSAEDIQEELAELEPKLTQEEIEQIS
NLTGYTGTHRLSLKAINLILDELWNTSDNQMTIFNRLKLVPKKVDLSQQKEIPTSL
VDDFILSPVVKRSFIQSIKVINAIIKKFGLPKDIIIELAREKNSKEAQKFINEMQKRNR
QTNERIEKIIKETGKEKAKFLIEKIKLHDMQEGKCLYSLESIPLEDLLNNPYHYEVD
HIIPRSVSFDNSFNNKVLVKQEENSKKGNRTPFQYLSSSDAKISYETFKKHILNLSK
GKGRVSKKKKEYLLEERDINRFSVQKDFINRNLVDTRYATRELMNLLRSYFRVND
LDVKVKSINGGFTSFLRTKWKFKKERNQGYKHHAEDALVIANADFIFKEWKKLDT
TNKVMENQTVEEQQAENMPGIETDDEYKEIFVIPRQIQSIKDFKDYKYSHRVDKKP
NRELVNDTLYSTRKDDKGNTLIINNIKGLYDKDNDKLKNLIKKSPEKLLMYHHDP
QTYQKLKTIMEQYSNEKNPLYKYHEETGNYLTKYSKKDNGPIIKKVKYYGKKLN
AHLDITNDYSNSQNKIVKLSLKPYRFDVYLDNGGYKFVTVKNLDVIKKEGFFKIDS
NAYEKAKSEKKIDENAVFIASFYNNDLIKIDGELYRIVGVNNDTRNVVELNMIPITY
KEYLENINDKRTPRILKTISQKTYSIEKYSTDILGNLYKVKSKKKPQMIMKGKRPA
ATKKAGQAKKKKGSYPYDVPDYAYPYDVPDYAYPYDVPDYA (SEQ ID NO: 18)
(D10A mutation, bold underlined italics)
Streptococcus intermedius B196 strain G1552 (SinCas9) with Nuclear Localization
Signal (NLS) and Linker
MPKKKRKVGTMNGLVLGLAIGIASVGVGILNKETGEIIHVNSRIFPAATADSNVER
RGFRQGRRLGRRKKHRSARLNDLFEEFGFITDFSAVPLNLNPYALRVKGLSEELTN
EELFIALKNIIKRRGISYLDDASEDGETASNEYGKAVEENRKLLADKTPGQIQLERF
EKYGQVRGDFTVVENGENHRLINVFSTSAYKKEAERILRRQQEFNVRISDEFIEAYL
TILTGKRKYYHGPGNEKSRTDYGRFRTDGTTLDNIFGILIGKCTFYPDEYRAAKAS
YTAQEFNLLNDLNNLTVPTETKKLRPEQKRQIVEYARTAKTLGTPTLLKYIAKLVD
GSIDDIKGYRIDKSDKPEMHTFDAYRKMRTLELVDVDILSRETLDDLAYILTLNTES
EGILEALNSKMPGTFTKEQIDELIQFRKKNSAVFGKGWHNFSLKLMNELISELYETS
EEQMTILTRLGKQRSREISKRTKYIDEKELTDEIYNPVVAKSVRQAIKIINLATKKY
GIFDNIVIEMARESNEDDEKKAIQNVQKANEDEKKAAMEKAADLYNGKKELPDSI
FHGHKELATKIRLWHQQGERCLYTGKNISIHDLIHNPHQYEIDHILPLSLSFDDGLA
NKVLVLATANQEKGQRTPFQALDSMDDAWSYIEFKQYVRNSKSLSNKKKDYLLT
EEDISKIEVKQKFIERNLVDTRYSSRVVLNTLQEFYKTNDFDTKISVVRGQFTSQLR
RKWKIEKSRDTYHHHAVDALIIAASSQLRLWKKQNNPLISYREGQLVDPETGEILS
LTDDEYKELVFRPPYDYFVDTLKSKSFEDSILFSYQVDSKYNRKISDATIYGTRKAQ
LGKDKQEETYVLGKIKDIYSQKGYEDFIKRYKKDTTQFLMYHKDPQTFAKVIEEIL
KTYPDKELNEKGKEIPCNPFEKYRQENGPIRKYSKKGKGPEIKSLKYYDNKLGNHI
DITPVNSQNQVVLQSLKPWRTDVYFNPQTSKYELMGLKYSDLRFEKGSGSYGISPE
KYNKVKAKEGVDEDSEFKFTLYKNDLILIKDTETGEQQLFRYGSRNDTSKHYVEL
KPYEKAKFEGNQQLMNLLGTVAKGGQCLKGINKPNLSIYKVKTDVLGNKHFIKKE
GDQPQLNFKKKIKRPAATKKAGQAKKKKGSYPYDVPDYAYPYDVPDYAYPYDVPD
YA (SEQ ID NO: 19)(D9A mutation, bold underlined italics)
Streptococcus sanguinis SK330 (SsaCas9) with Nuclear Localization Signal (NLS) and
Linker
MPKKKRKVGTMENKNYSIGLAIGTNSVGWAVITDDYKVPSKKMKVFGNTDKHFI
KKNLIGALLFDEGATAEDRRLKRTARRRYTRRKNRLRYLQEIFSEEISKLDSSFFHR
LDDSFLVPKDKRGSKYPIFATLEEEKEYHKKFPTIYHLRKHLADSKEKTDLRLIYLA
LAHMIKYRGHFLYEESFDIKNNDIQKIFNEFISIYDNTFEGSSLSGQNAQVEAIFTDKI
SKSAKRERVLKLFSDEKSTSLFSEFLKLIVGNQADFKKHFDLEEKAPLQFSKDTYDE
DLENLLGQIGDGFTDLFLVAKKLYDAILLSGILTVTDPSTKAPLSASMIERYESHQK
DLAALKQFIKNNLPKRYNEVFSDQSKDGYAGYIDGKTTQEAFYKYIKNLLSKFEG
ADYFLDKIEREDFLRKQRTFDNGSIPHQIHLQEMNAILRRQGEHYPFLKENREKIEK
ILTFRIPYYVGPLARGNRDFAWLTRNSDQAIRPWNFEEIVDKASSAEEFINKMTNY
DLYLPEEKVLPKHSLLYETFAVYNELTKVKFIAEGLRDYQFLDSGQKKQIVNQLFK
EKRKVTEKDIIHYLHNVDGYDGIELKGIEKQFNASLSTYHDLLKIIKDKEFMDDPK
NEEILENIVHTLTIFEDREMIKQRLAQYDSIFDEKVIKALTRRHYTGWGKLSAKLIN
GICDKQTGDTILDYLIDDGKINRNFMQLINDDGLSFKEIIQKAQVVGKTDDVKQVV
QELPGSPAIKKGILQSIKIVDELVKVMGYAPESIVIEMARENQTTARGKKNSQQRY
KRIEDSLKNLAPGLDSNILKENPTDNIQLQNDRLFLYYLQNGKDMYTGKPLDIDQL
SSYDIDHIIPQAFIKDDSIDNRVLTSSKDNRGKSDNVPSLEVVQKRKAFWQQLLDSK
LISERKFNNLTKAERGGLDERDKVGFIRRQLVETRQITKHVAQILDASFNTEVNEK
NQKIRTVKIITLKSNLVSNFRKEFELYKVREINDYHHAHDAYLNAVVAKAILKKYP
KLEPEFVYGDYQKYDLKRYISRFKPSKEIEKATEKYFFYSNLLNFFKEEVHYADGII
VKRENIEYSKDTGEIAWNKEKDFATIKKVLSYPQVNIVKKTEIQTHGLDRGKPKGL
FNSNPSPKPSEDSKENLVPIKQGLDPRKYGGYAGISNSYAVLVKAIVEKGAKKQQK
TILEFQGISILDKINFENNKENYLLKKRYIEILSTITLPKYSLFEFPDGTRRRLASILST
NNKRGEIHKGNELVLPGKYTTLLYHAKNINKKLEPEHLEYVEKHRNDFAKLLECV
LNFNDKYVGALKNGERIRQAFTDWETVDIEKLCFSFIGPENSKNAGLFELTSQGSA
SDFEFLGVKIPRYRDYAPSSLLKATLIHQSITGLYETRIDLSKLGEDKRPAATKKAG
QAKKKKGSYPYDVPDYAYPYDVPDYAYPYDVPDYA (SEQ ID NO: 20)(D11A
mutation, bold underlined italics)
Streptococcus sp. C150 (Ssc2Cas9) with Nuclear Localization Signal (NLS) and Linker
MPKKKRKVGTMSDLVLGLAIGIGSVGVGILNKVTGEIIHKNSRIFPAAQAENNLER
RTNRQGRRLTRRKKHRRVRLNHLFEESGLITDFTKVSINLNPYQLRVKGLTAELSN
EELFIALKNMVKHRGISYLDDASDDGNSSVGDYAQIVKENSKQLETKTPGQIQLER
YQKYGQLRGDFTVEEDGRKHRLINVFPTSAYHAEALRILQTQQEFNPQITDEFINSY
LEILTGKRKYYHGPGNEKSRTDYGKYTTKKDAQGQYITLNNIFGILIGKCTFYPEEY
RAAKASYTAQEFNLLNDLNNLTVPTETKKLSEEQKYQIITYVKNEKAMGPAKLFK
YIAKLLSCDVADIKGYRIDKSDKAEIHTFEAYRKMKTLETLDVKKMAREELDKLA
YVLTLNTEREGIQEALDHEFADGTFSQEQVDELVQFRKANSSIFGKGWHSFSVKL
MMELIPELYATSEEQMTILTRLGKQKTTSSSNKTKYIDEKQLTEEIYNPVVAKSVR
QAIKIVNAAIKKYGDFDNIVIEMARETNEDDEKKAIQKIQKANKAEKDAAMRKAA
NQYNGKAELPHSVFHGHKQLATKIRLWHQQGERCLYTGKTISIHDLINNPNQFEID
HILPLSITFDDSLANKVLVYATANQEKGQRTPYQALDSMDDAWSFRELKAFVRES
KALSNKKKEYLLTEEDISKFDVRKKFIERNLVDTRYASRVVLNALQEHFRTHKIDT
KVSVVRGQFTSQLRRHWGIEKTRDTYHHHAVDALIIAASSQLNLWKKQKNTLVSY
SEDQLLDIETGELISDDEYKESVFKAPYQHFVDTLKSKEFEDSILFSYQVDSKFNRKI
SDATIYATRKAKLDKEKKEYTYTLGKIKDIYALGTKTPSKTGFYKFLDLYKKDKSQ
FLMYQKDRRTWDEVIEKILEQYRPFKEKDKNGKEVDFNPFEKYRIENGPIRKYSRK
GNGPEIKSLKYYDNLLGRFVDITPSESKNPVALLSLNPWRTDVYYNTETRKYEFLG
LKYADLCFEKGGSYGISKVKYNKIREKEGIGKNSEFKFTLYKNDLILIKDTETNRQQ
IFRFWSRTGKDNPKSFEKHKLELKPYEKTRFEKGEELKVLGKVPPSSNRLQKNMQI
ENLSIYKVRTDVLGNQHIIKNEGDKPKLDFKRPAATKKAGQAKKKKGSYPYDVP
DYAYPYDVPDYAYPYDVPDYA (SEQ ID NO: 21)(D9A mutation, bold underlined italics)
Streptococcus oralis subsp. oralis strain RH_1735_08 (Sor2Cas9) with Nuclear
Localization Signal (NLS) and Linker
MPKKKRKVGTMNGLVLGLAIGIASVGVGILEKNSGKIIHANSRIFPAATADNNVER
RKNRQARRLHRRKKHRGVRLQDIFEDYGLLTDFSKVSINLNPYRLRVDGLDQQLT
NEELFIALKNIVKRRGISYLDDASEDGGTVSSDYGKAVEENRKLLAEKTPGQIQLE
RFEKYGQVRGDFTVVENGEKRRLINVFSTSAYRKEAERILRKQQEFNSKITDEFIED
CLKILTGKRKYYHGPGNEKSRTDYGRFRTDGTTLDNIFGILIGKCTFYPNEYRASK
ASHTAQEFNLLNDLNNLTVPTETKKLSEEQKKVIVEYAKEAKTLGASTLLKYIAK
MIDASVDQISGYRVDVNNKPEMHTFEVYRKMQSLETISVGELSRNVLDELAHILTL
NTEREGIEEAINTKLKDSFSQDQVLELVQFRKNNSSLFSRGWHNFSLKLMMELIPEL
YETSEEQMTILTRLGKQKSKETSKRTKYIDEKELTEEIYNPVVAKSVRQAIKIINEAT
KKYGIFDNIVIEMARENNEEDAKKDYIKRQKANQDEKNAAMEKAAFQYNGKKEL
PDNIFHGHKELATKIRLWHQQGEKCLYTGKNIPISDLIHNQYKYEIDHILPLSLSFDD
SLSNKVLVLATANQEKGQRTPFQALDSMDDGWSYREFKSYVKESKLLGNKKKEY
LLTEEDISKIEVKQKFIERNLVDTRYSSRVVLNALQDFYKAHKFDTTISVVRGQFTS
QLRRKWGLEKSRETYHHHAVDALIIAASSQLRLWKKQNNPLIAYKEGQFVDSETG
EILSLSDDEYKELVFKAPYDHFVDTLRSKTFEDSILFSYQVDSKYNRKISDATIYAT
RKAKLDKDKSEETYVLGKIKDIYSQAGYDAFIKIYNKDKSKFLMYHKDPQTFEKVI
EEILRTYPSKELNDKNKEIPCNPFEKYRQENGPIRKYSKKGNGPEIKCLKYYDNKLG
NYIDITPDGSDNQVVLQSIAPWRTDVYYNHKTGKYEFLGLKYSDLYFEKGTGKYK
ISKEKYDNIKKIEGVVETSEFKFTLYKNDLILIKDVEKGQEQLFRFLSRNNKGKHQV
QLKPMNKSDFEKGEKLIDIFGTVPNSTTQCVKGLNKSNISIFKVKTDVLGKKHIIKK
EGDEPKLKFKRPAATKKAGQAKKKKGSYPYDVPDYAYPYDVPDYAYPYDVPDYA
(SEQ ID NO: 22)(D9A mutation, bold underlined italics)
Streptococcus oralis SK313 (SorCas9) with Nuclear Localization Signal (NLS) and
Linker
MPKKKRKVGTMNGLVLGLAIGIASVGVGILEKNSGKIVHASSRIFPAATADNNVE
RRKNRQARRLHRRKKHRGARLKDLFEYYGLLTDFSKVSINLNPYRLRVDGLDQQL
TNEELFIALKNIVKRRGISYLDDASEDGGTVSSDYGKAVEENRKLLAEQTPGQIQLE
RFEKYGQLRGDFTVVENSEKCRLINVFSTSAYKKEAERILRKQQEFNNQITDEFIED
YLKILTGKRKYYHGPGNEKSRTDYGRFRTDGATLDNIFGILIGKCTFYPNEYRASK
ASYTAQEFNLLNDLNNLTVPTETKKLSEEQKKTIIEYAKSAKTLGASTLLKYIAKMI
DASVDQIRGYRVDVNNKPEMHTFEVYRKMQSLETISVGELSRNILDELAHILTLNT
EREGIEEAINTKLRDSFSQDQVLELVQFRKNNSSLFSKGWHNFSLKLMMELIPELYE
TSEEQMTILTRLGKQKSKETSKRTKYIDEKEVTEEIYNPVVAKSVRQAIKIINEATK
KHGIFDNIVIEMARENNEEDAKKDYIKRQKANQDEKYAAMEKAAFQYNGKKELP
DNIFHGHKELATKIRLWHQQGEKCLYTGKSIPISDLIHNQYKYEIDHILPLSLSFDDS
LSNKVLVLATANQEKGQRTPFQALDSMDDAWSYREFKSYVKESKLLGNKKKEYL
LTEEDISKIEVKQKFIERNLVDTRYSSRVVLNALQDFYKNHNFDTTISVVRGQFTSQ
LRRKWGLEKSRETYHHHAVDALIIAASSQLRLWKKQNNPLIAYKEGQFVDSQTGE
IISLTDDEYKELVFKAPYDHFVDTLSSKTFEDSILFSYQVDSKFNRKISDATIYATRK
AKLDKEKKEYTYTLGKIKDIYSLGTKTPSKTGFYKFLDLYNKDKSQFLMFQKDRK
TWDEVIEKIMEQYRPFKEYDKAGKLVDFNPFEKYRQENGPIRKYSKKGNGPEIKSL
KYYDILLGKHKNITPEGSRNTVALLSLNPWRTDVYYNMETKKYEFLGLKYADLPF
EEGGAYGISTETYNELREKEGIGKNSEFKFTLYKNDLILIKDTETNCQQFFRFWSRT
GKDNPKSFEKHKIELKPYEKAKFEKGEELEVLGKVPPSSNQFQKNMQIENLSIYKV
KTDVLGNKHFIKKEGDKPKLKFKRPAATKKAGQAKKKKGSYPYDVPDYAYPYDV
PDYAYPYDVPDYA (SEQ ID NO: 23)(D9A mutation, bold underlined italics)
Staphylococcus warneri strain 691 (SwaCas9) with Nuclear Localization Signal (NLS)
and Linker
MPKKKRKVGTMKEKYILGLALGITSVGYGIINFETKKIIDAGVRLFPEANVDNNEG
RRSKRGSRRLKRRRIHRLDRVKSLLTEYNLINREQIPTSNNPYQIRVKGLSEILSKDE
LAIALLHLAKRRGIHNINVSSEDEDASNELSTKEQINRNNKLLKNKYVCEVQLQRL
KEGQIRGEKNRFKTTDILKEIDQLLKVQKDYHNLDIDFINQYKEIVETRREYFEGPG
QGSPFGWNGDLKKWYEMLMGHCTYFPQELRSVKYAYSADLFNALNDLNNLIIQR
NDSEKLEYHEKYHIIENVFKQKKKPTLKQIAKEIGVNPEDIKGYRITKSGTPQFTEF
KLYHDLKSIVFDKSILENEAILDQIAEILTIYQDEESIKEELNKLPEILNEQDKAEIAK
LTGYNVTHRLSLKCIHLINEELWQTSRNQMEIFNYLNIKPNKVDLSEQNKIPKDLV
DEFILSPVVKRTFIQSINVINKVIEKYGIPEDIIIELARENNSDDRKKFINNLQKKNEA
TRKRINEIIGQTGNQNGKRIVEKIRLHDQQEGKCLYSLESIPLMDLLNNPQNYEVDH
IIPRSVAFDNSIHNKVLVKQIENSKKGNRTPYQYLNSSDANLSYNQFKQHILNLSKS
KDRISKKKKDYLLEERDINKFEVQKEFINRNLVDTRYATRELTSYLKAYFSANNM
DVKVKTINGSFTNHLRKVWRFDKYRNHSYKHHAEDALIIANADFLFKENKKLQN
ANKILEKPTIENDTQKVTVEKEEDYNNMFETPKLVEDIKQYRDYKFSHRVDKKPN
RQLIKDTLYSTRMKDEHNYIVQTITDIYGKDNTNLKKQFNKNPEKFLMYQNDPKT
FEKLSIIMKQYSDEKNPLAKYYEETGEYLTKYSKKNNGPIVKKIKLLGNKVGNHLD
VTNKYENSTKKLVKLSIKNYRFDVYLTEKGYKFVTIAYLNVFKKDNYYYIPKDLY
QELKAKKKIKDTDQFIASFYKNDLIKLNGDLYKIIGVNSDDRNIIELDYYDIKYKDY
CEINNIKGEPRIKKTIGKKTESIEKLTTDVLGNLYLHTTEKAPQLIFKRGLKRPAAT
KKAGQAKKKKGSYPYDVPDYAYPYDVPDYAYPYDVPDYA (SEQ ID NO: 24)(D10A
mutation, bold underlined italics)
Staphylococcus sciuri strain SNUC_2430 (SscCas9) with Nuclear Localization Signal
(NLS) and Linker
MPKKKRKVGTMKRNYILGLAIGITSVGYGIIDYETRDVIDAGVRLFKEANVENNE
GRRSKRGARRLKRRRRHRLQRVKKLLFDYNLLTDHSELSGINPYEARVKGLSQKL
SEVEFSAALLHLAKRRGVHNVNEVEEDTGNELSTKEQISRNSKALEEKYVAELQLE
RLKTDGEVRGPNNRFKTSDYVKEAKQLLKVQKAYHQLDQSFIDTYIDLLETRRTY
YEGPGEGSPFGWKDIKEWYEMLMGHCTYFPEELRSVKYAYNADLYNALNDLNNL
VIARDENEKLEYYEKFQIIENVFKQKKKPTLKQIAKELLVNEEDIKGYRVTSIGKPE
FTNFKIYHDIKGITERKEVLENAELLDQIAEILTIYQSSEDVQEELANLNSELTQEEIE
QISNLKGYTGTHNLSLKAINLILDELWHTNDNQMAIFNRLKLVPKKVDLSQQKEIP
TTLVDDFILSPVVKRSFIQSIKVINAIIKKYGLPNDIIIELAREKNSKDAQKMINEMQK
RNRQMNERIEEIIRTTGKENAKYLIEKIKLHDMQEGKCLYSLESIPLEDLLNNPFNY
EVDHIIPRSVSFDNSFNNKVLVKQEENSKKGNRTPFQYLSSSDSKISYETFKKHILNL
AKGKGRISKTKKEYLLEERDINRFSVQKDFINRNLVDTRYATRELMNLLRSYFRVN
NLDVKVKSINGGFTSFLRRKWKFKKERNKGYKHHAEDALIIANADFIFKEWKKLD
KAKKVMENQTVEEKQAESMPEIETEQEYKEIFITPHQIQHIKGFKDYKYSHRVDKK
PNRELINDTLYSTRKDDKGNTLIVNNLNGLYDKDNDKLKKLMNKSPEKLLMYHH
DPQTYQKLKLIMEQYGDEKNPLYKYYEETGNYLTKYSKKDNGPVIKKIKYYGKN
LKAHLDITDDYPNSRNKVVKLSVKPYRFDVYLDNDIYKFVTVKNLDVIKKEDYYE
VNSKCYKEAKKLKKISDQAEFIASFYNNDLIKINGELYRVIGVNNDLLNRIEVNMIN
ITYREYLENMNDKRPPRIIKTIASKTQSIKKYSTDILGNLYEVKSKQKPQMIMKGKR
PAATKKAGQAKKKKGSYPYDVPDYAYPYDVPDYAYPYDVPDYA (SEQ ID NO: 25)
(D10A mutation, bold underlined italics)
Streptococcus gallolyticus strain AM24-4 (SgaCas9) with Nuclear Localization Signal
(NLS) and Linker
MPKKKRKVGTMTNGKILGLAIGIASVGVGIIEAKTGKVVHANSRLFSAANAENNT
ERRGFRGSRRLNRRKKHRVKRVRDLFEKHEIVTDFRNLNLSPYELRVKGLTEQLT
NEELFAALRTISKRRGISYLDDAEDDSTGSTDYAKSIDENRRLLKNKTPGQIQLERL
EKYGQLRGNFTVYDENGEAHRLINVFSTSDYEKEARKILETQADYNKKITAEFIDD
YVEILTQKRKYYHGPGNEKSRTDYGRFRTDGTTLENIFGILIGKCNFYPDEYRASK
ASYTAQEYNFLNDLNNLKVPTETGKLPTEQKESLVEFAKNTATLGPSKLLKEIAKI
LDCKVDEIKGYREDDKGKPDLHTFEPYRKLKFNLESINIDDLSREVIDKLADILTLN
TEREGIEDAIKRNLPNQFTEEQISEIIKVRKSQSTAFNKGWHSFSAKLMNELIPELYA
TSDEQMTILTRLEKFKVNKKSSKNTKTIDEKEVTDEIYNPVVAKSVRQTIKIINAAV
KKYGDFDKIVIEMPRDKNADDEKKFIDKRNKENKKEKDDALKRAAYLYNSSDKL
PDEVFHGNKQLETKIRLWYQQGERCLYSGKPIPIHDLVHNSNNFEIDHILPLSLSFD
DSLANKVLVYDWANQEKGQKTPYQVIDSMDAAWSFREMKDYVLKQKGLGKKK
RDYLLTTENIDKIEVKKKFIERNLIDTRYASRVVLNSLQSALRELCKDTKVSVIRGQ
FTSQLRRKWKIDKSRETYHHHAVDALIIAASSQLKLWEKQDNLMFIDYGNNQVVD
KETGEILSVSDDEYKELVFQPPYQGFVNTISSKGFEDEILFSYQVDSKYNRKVSDAT
IYSTRKAKIGKDKKEETYVLGKIKDIYSQNGFDTFIKKYNKDKTQFLMYQKDPLT
WENVIEVILRDYPTTKKSEDGKNDVKCNPFEEYRRENGLICKYSKKGKGTPIKSLK
YYDKKLGNCIDITPEESRNKVILQSINPWRADVYFNPETLKYELMGLKYSDLSFEK
GTGKYHISQEKYDAIKEKEGIGKKSEFKFTLYRNDLILIKDTASGEQEIYRFLSRTMP
NVNHYAELKPYDKEKFDGGQELMEVFGKVANGGQCLKSLNKSNISIYKVRTDVL
GNKYFVKKEGDKPKLNFKNNKKKRPAATKKAGQAKKKKGSYPYDVPDYAYPYD
VPDYAYPYDVPDYA (SEQ ID NO: 26)(D10A mutation, bold underlined italics)
Lactobacillus kullabergensis strain Biut2 (LkuCas9) with Nuclear Localization Signal
(NLS) and Linker
MPKKKRKVGTMKRVNEDYILGLAIGTNSCGWAVTDKKNNLLKLRGKTAIGSHLF
EEGHTAADRRGFRTTRRRLKRRKWRLRLLEEIFAEPMAKVDPGFFVRLHQSWVSP
LDKDRKKYNAIVFPTAKEDQAFYKHYATIYHLRDELMTQDRQFDLREIFLAIHHIV
KYRGNFLQDTPVKDFEASKIEVGPILSHINNAFAEKIVEDQDPIELNVANAADIEDVI
RGKDAEKTVYKLDKVKKIAKLLTDSTAKEEKNVAKQIANAIMGYKTQFETILDKE
IDKSDKAQWEFKLSDADADDKLDALLPDLDETDQTVVAEIEKLFSAITLSTIVDEN
KSLSQSMVEKYKKHKKDYKKLKKYINTLQDQTKAKKLLLAYDLYVNNRHGRLL
EAKKTFEDKKKKKALTKDEFYKIVKDNLDDSDLAHEIQQEIAADNFMPKQRTNSN
GVIPFQLHQIELDKIIANQGKYYPFLAAENPVEDHRKQAPYKLDELVRFRVPYYVG
PMITADEQEKTSGKSFAWMVRKEDGQITPWNFEQKVDRQESANKFIKRMTIKDTY
LLSEDVLPANSLLYQRFEVLNELNNIRINGSRISVDLKQQIFNDLFEEKKTVTEKSLT
SYLKQNLHLPTVEIKGLADPTKFNSSLASYYHLKSLHVFDKELADPQYQKDFEKIIE
YSSIFEDKKIFQDKLHAEFKWLTPEQFKAISTWRLQGWGRLSRKLLVELHDTNGQ
NIMEQLWDSQKNFMQIVTEPDFKDAIAKENQNVTRANGVEEILADAYTSPANKKA
IRQVVKVVADVVKAAGGKKPAQFAIEFTRDPDKNPQLSHIRGTKLLKAYQETAGE
LVDQKLTDSLKEAMTSRKLLKDKYFLYFMQAGRDAYTGQKINIDEVSTNYQIDHI
LPQSFIKDDSFDNRVLTATPLNAEKSDDVPYKRFANNYVSDMKMTVGEMWKHW
QKAGIINKHKLGNLLLDPDRLNKFQKSGFINRQLVETSQIIKLVSVILQNKYPDAEII
TVKAGDNSALRQRLNLYKSRDVNDYHHAIDAYLSIICGNFLYQVYPKYRPYFVYG
KYKKFSQDPDLQKEVIKHFKGFTFMWPLLQKDNSERKAPEKIKENNSDRIVFYKHP
DIFDKLRKAYNYKYMLVSRETTTENSGLFDVTIYPRGERDLAKTRKLIPKSNGLDP
KIYGGYSGNTDAYMVIVKIDKGKESIYKVIGVPMRALASLNRAKKQGNYKEELHQ
VLEPQIMFDKNGKPKRSVKGFRIIKDHVPFKQVVLDGDKKFMLNSSTYEINAKQLT
LTPETMRIVTDNLKKGEDQDQLLVKAYDEILQKVDQYLPLFDVNKFRNSLHLGRA
KFLDLAVNDKKITLTNILNGLHDNLVTPDLKNIGIKTPLGKLQVPSGIVLSSEAILIF
QSPTGLFEKRVRIADLKRPAATKKAGQAKKKKGSYPYDVPDYAYPYDVPDYAYPY
DVPDYA (SEQ ID NO: 27)(D13A mutation, bold underlined italics)
Streptococcus suis strain LSS83 (SsuCas9) with Nuclear Localization Signal (NLS)
and Linker
MPKKKRKVGTMSNGKILGLAIGIASVGVGVIDAQTGEIIHASSRIFPSANAANNAE
RRTFRGSRRLIRRKKHRIKRLDDLFNDFHINLDGEMSTDNPYVLRVKGLSQKLTVE
ELYISIKNIMKRRGISYLDDAESDNEAGRSDYAKAIERNRQLLTSKTPGEIQLERLE
KYGQLRGNFTIIDEEGQSQQIINVFSTSDYVKEVEKILDCQKMYHKFISDEFCDKLIE
LLREKRKYYVGPGNEKSRTDYGIYRTDGTTLENLFGILIGKCTFYPDQYRSSRASY
TAQEFNFLNDLNNLTVPTETKKLSQEQKEFLVNYAKETSVLGAGKILQQIAKLADC
KVEDIRGYRLDNKDKPELHTFETYRAMKGLVPLVDIGVLSREQLDILADILTLNTD
FEGIREALKKQLPNVFDEKQVKGLASFRKSKSQLFAKGWHNLSQKIMLEVIPELYA
TSDEQMTILTRLGKFEKSSVAEYPSSINVDEITDEIYNPVVAKSIRQTIKIINASIKKW
DEFDQIVIEMPRDRNEDEEKKRIADGQKANAKEKADSILRAAELYCAGKVLPDYV
YNGHNQLATKIRLWYQQGERCIYTGQPISIHDLIHNQNQYEIDHILPLSLTFDDSLS
NKVLVLATANQEKAQRTPYNYLKSATSAWSYREFKDYVTKRKGIGKKKCEYLTF
EEDINGFEVRSKFIQRNLVDTRYASKVILNALQDYFKISGIQTKVSVVRGQFTSQLR
HKWGIEKTRETYHHHAVDALIIAASSQLRLWKKQESPLVVDYQEGRQVDLETGEI
LELTDEQYKELVYQPPYQGFVNTISSSAFDNEILFSYQVDSKVNRKISDATIYATRN
AQLGKDKTEGIYVLGKIKDIYTQAGYEAFLKRYTKDKTSFLMYHKDLDTWEKVIE
IILRDYREYDEKGKEIGNPFERYRRENGYVKKYSRKGNGTAIKSLKYYDNKLGNHI
DITPENSRNAVVLQSLKPWRTDVYFNKETGKYEFLGIKYSDLSFEKGTGEYGISQE
KYDSIKIAEGVAKKSIFKFTLYKQDLLFIKDIENNFGKLLRFTSKNDTSKHYVELKP
YDKNKFGTEEPLLPVLGNVAKSGQCIKGLNKSNISIYKVRTDILGYRHFIKQEGEHP
QLKFKKKRPAATKKAGQAKKKKGSYPYDVPDYAYPYDVPDYAYPYDVPDYA (SEQ
ID NO: 28)(D10A mutation, bold underlined italics)
NLS (bold), can be substituted with different NLSs
Linker (underlined), can be removed or extended
3xHA tag (italics), can be substituted with different tags

In some embodiments, recombinant engineered non-naturally occurring human codon-optimized Cas9 comprises a sequence having at least 60%, 70%, 80%, 85%, 90%, 95%, 97%, 98%, 99% sequence identity to SEQ ID NO: 15, 16, 17, 18, 19, 20, 21, 22, 23, 24, 25, 26, 27 or 28. In some embodiments, recombinant engineered non-naturally occurring human codon-optimized Cas9 comprises a sequence having at least 60%, 70%, 80%, 85%, 90%, 95%, 97%, 98%, 99% sequence identity to SEQ ID NO: 15, 16, 17, 18, 19, 20, 21, 22, 23, 24, 25, 26, 27 or 28. In some embodiments, recombinant engineered non-naturally occurring human codon-optimized Cas9 comprises a sequence having at least 60%, 70%, 80%, 85%, 90%, 95%, 97%, 98%, 99% sequence identity to SEQ ID NO: 15, 16, 17, 18, 19, 20, 21, 22, 23, 24, 25, 26, 27 or 28. In some embodiments, recombinant engineered non-naturally occurring human codon-optimized Cas9 comprises a sequence having at least 60%, 70%, 80%, 85%, 90%, 95%, 97%, 98%, 99% sequence identity to SEQ ID NO: 15, 16, 17, 18, 19, 20, 21, 22, 23, 24, 25, 26, 27 or 28.

Various species exhibit codon bias (i.e. differences in codon usage by organisms) which correlates with the efficiency of translation of messenger RNA (mRNA) by utilizing codons in mRNA that correspond with the abundance of tRNA species for that codon in a particular organism. Various methods in the art can be used for computer optimization, including for example through use of software. In some embodiments, codon optimization refers to modification of nucleic acid sequences for enhanced expression in the host cells of interest by replacing at least one codon (e.g. 1, 2, 3, 4, 5, 10, 15, 20, 25, 50 or more codons) of the native sequence with codons that are more frequently used or most frequently used in the genes of the host cell while maintaining the native amino acid sequence.

In some embodiments, the Cas9 protein described herein is codon optimized. This type of optimization is known in the art and entails the mutation of foreign-derived DNA to mimic the codon preferences of the intended host organism or cell while encoding the same protein. Thus, the codons are changed, but the encoded protein remains unchanged. Codon optimization improves soluble protein levels and increases activity and editing efficiency in a given species. Codon optimization also results in increased translation and protein expression.

In some embodiments, the Cas9 protein is codon optimized for expression in eukaryotic cells. In some embodiments, the Cas9 protein is codon optimized for expression in human cells.

### Protospacer Adjacent Motif (PAM)

Each Cas endonuclease binds to its target sequence only in the presence of a specific sequence, known as a protospacer adjacent motif (PAM), on the non-targeted i.e. complementary DNA strand. Cas nucleases isolated from different bacterial species recognize different PAM sequences. For example, Cas9 engineered from Streptococcus equinus ATCC 33317 cuts upstream of the consensus PAM sequence 5′-NRGNR-3′ (where “N” can be any nucleotide base and R is A or G), Enterococcus hirae strain F1129E recognizes the consensus PAM sequence 5′-NRG-3′, Streptococcus equinus strain AG46, Staphylococcus warneri strain 691, and Staphylococcus sciuri strain SNUC 2430 recognize the consensus PAM sequence 5′-NNGR-3′, Staphylococcus simulans strain 19 recognizes the consensus PAM sequence 5′-NNGRRT-3′, Streptococcus intermedius B196 strain G1552 recognizes the consensus PAM sequence 5′-NNAAAA-3′, Streptococcus sanguinis SK330 recognizes the consensus PAM sequence 5′-NGGNG-3′, Streptococcus sp. C150 recognizes the consensus PAM sequence 5′-NNGNRG-3′, Streptococcus oralis subsp. oralis strain RH_1735_08 recognizes the consensus PAM sequence 5′-NNAAAC-3′, Streptococcus oralis SK313 recognizes the consensus PAM sequence 5′-NNRAAG-3′, Streptococcus gallolyticus strain AM24-4 recognizes the consensus PAM sequence 5′-NNAYAA-3′, Lactobacillus kullabergensis strain Biut2 recognizes the consensus PAM sequence 5′-NNGAAA-3′, and Streptococcus suis strain recognizes the consensus PAM sequence 5′-NNAAA-3′ in the target (N=any nucleotide base; H=A, C or T; R=A or G). Thus, the locations in the genome that can be targeted by different Cas proteins are limited by the locations of unique PAM sequences.

In some embodiments, the target nucleic acid is 5′ or upstream of the PAM sequence. Accordingly, the Cas9 protein described herein exhibits activity, for example, binding, cleavage, modification, or altered gene expression in the presence of a unique PAM sequence.

In some embodiments, the Cas9 protein described herein does not bind or exhibit activity with any other PAM sequences.

### RNA Guides

An RNA guide comprises a polynucleotide sequence with complementarity to a target sequence. The RNA guide hybridizes with the target nucleic acid sequence and directs sequence-specific binding of a CRISPR complex to the target nucleic acid. In some embodiments, an RNA guide has 50%, 60%, 70%, 75%, 80%, 85%, 90%, 95%, 96%, 97%, 98%, 99% or 100% complementarity to a target nucleic acid sequence.

In some embodiments, the RNA guides are about 5, 10, 11, 12, 13, 14, 15, 16, 17, 18, 19, 20, 21, 22, 23, 24, 25, 26, 27, 28, 29, 30, 35, 40, 45, 50, 75 or more nucleotides in length. In some embodiments, the RNA guides are about 18-24 nucleotides in length. In some embodiments, the RNA guide is complementary to about 18-24 nucleotides in the target nucleic acid sequence. For example, the RNA guide is complementary to about 18, 19, 20, 21, 22, 23, or 24 nucleotides in the target nucleic acid sequence. In some embodiments, the RNA guide is complementary to about 18-22 nucleotides. In some embodiments, the RNA guide is complementary to about 18-21 nucleotides. In some embodiments, the RNA guide is complementary to about 18-20 nucleotides. In some embodiments, the RNA guide is complementary to 20 nucleotides in the target nucleic acid sequence.

An RNA guide can be designed to target any target sequence. Optimal alignment is determined using any algorithm for aligning sequences, including the Needleman-Wunsch algorithm, Smith-Waterman algorithm, Burrows-Wheeler algorithm, ClustlW, ClustlX, BLAST, Novoalign, SOAP, Maq, and ELAND.

In some embodiments, an RNA guide is targeted to a unique target sequence within the genome of a cell. In some embodiments, an RNA guide is designed to lack a PAM sequence. In some embodiments, an RNA guide sequence is designed to have optimal secondary structure using a folding algorithm including mFold or Geneious. In some embodiments, expression of RNA guides may be under an inducible promoter, e.g. hormone inducible, tetracycline or doxycycline inducible, arabinose inducible, or light inducible.

In some embodiments, the CRISPR system includes one or more RNA guides e.g. crRNA, tracrRNA, and/or sgRNA. Accordingly, in some embodiments the RNA guide comprises a crRNA. In some embodiments, the RNA guide comprises a tracrRNA. In some embodiments, the RNA guide comprises a sgRNA. In some embodiments, the CRISPR system includes multiple RNA guides, comprising 1, 2, 3, 4, 5, 6, 7, 8, 9, 10, 15 or more RNA guides.

In some embodiments, the RNA guide includes a crRNA. In some embodiments, the CRISPR system includes multiple crRNAs comprising 2-15 crRNAs. In some embodiments, the crRNA is a precursor crRNA (pre-crRNA), which includes a direct repeat sequence, a spacer sequence and a direct repeat sequence. In some embodiments, the crRNA is a processed or mature crRNA which includes a truncated direct repeat sequence.

In some embodiments, a CRISPR associated protein cleaves the pre-crRNA to form processed or mature crRNA.

In some embodiments, a CRISPR associated protein forms a complex with the mature crRNA and the spacer sequence targets the complex to a complementary sequence in the target nucleic acid. In some embodiments, an RNA guide comprises a direct repeat sequence and a spacer sequence capable of hybridizing under appropriate conditions to a target nucleic acid.

In some embodiments, the spacer length of crRNAs can range from about 15 to 50 nucleotides. In some embodiments, the spacer length of an RNA guide is at least 16 nucleotides, at least 17 nucleotides, at least 18 nucleotides, at least 19 nucleotides, at least 20 nucleotides, at least 21 nucleotides, or at least 22 nucleotides. In some embodiments, the spacer length is from 15 to 17 nucleotides (e.g., 15, 16, or 17 nucleotides), from 17 to 20 nucleotides (e.g., 17, 18, 19, or 20 nucleotides), from 20 to 24 nucleotides (e.g., 20, 21, 22, 23, or 24 nucleotides), from 23 to 25 nucleotides (e.g., 23, 24, or 25 nucleotides), from 24 to 27 nucleotides, from 27 to 30 nucleotides, from 30 to 45 nucleotides (e.g., 30, 31, 32, 33, 34, 35, 36, 37, 38, 39, 40, 41, 42, 43, 44, or 45 nucleotides), from 30 or 35 to 40 nucleotides, from 41 to 45 nucleotides, from 45 to 50 nucleotides (e.g., 45, 46, 47, 48, 49, or 50 nucleotides), or longer.

In some embodiments, the RNA guide comprises a direct repeat (DR) sequence of between about 16 and 26 nucleotides long. For example, in some embodiments, the DR is about 16 nucleotides long. In some embodiments, the DR is about 17 nucleotides long. In some embodiments, the DR is about 18 nucleotides long. In some embodiments, the DR is about 19 nucleotides long. In some embodiments, the DR is about 20 nucleotides long. In some embodiments, the DR is about 21 nucleotides long. In some embodiments, the DR is about 22 nucleotides long. In some embodiments, the DR is about 23 nucleotides long. In some embodiments, the DR is about 24 nucleotides long. In some embodiments, the DR is about 25 nucleotides long. In some embodiments, the DR is about 26 nucleotides long.

In some embodiments, the crRNA comprises a nucleotide guide sequence and a DR sequence. The nucleotide guide sequence can be between about 18 and 24 nucleotides long. Accordingly, in some embodiments, the nucleotide guide sequence is about 18 nucleotides long. In some embodiments, the nucleotide guide sequence is about 19 nucleotides long. In some embodiments, the nucleotide guide sequence is about 20 nucleotides long. In some embodiments, the nucleotide guide sequence is about 21 nucleotides long. In some embodiments, the nucleotide guide sequence is about 22 nucleotides long. In some embodiments, the crRNA comprises a nucleotide guide sequence of about 22 nucleotides long and a direct repeat of about 22 nucleotides long.

In some embodiments, the crRNA sequences can be modified to “dead crRNAs,” “dead guides,” or “dead guide sequences” that can form a complex with a CRISPR-associated protein and bind specific targets without any substantial nuclease activity.

In some embodiments, the crRNA may be chemically modified in the sugar phosphate backbone or base. In some embodiments, the crRNA maybe modified using 2′O-methyl, 2′-F or locked nucleic acids to improve nuclease resistance or base pairing. In some embodiments, the crRNA may contain modified bases such as 2-thiouridiene or N6-methyladenosine.

In some embodiments, the crRNA is conjugated with other oligonucleotides, peptides, proteins, tags, dyes, or polyethylene glycol.

In some embodiments, the crRNA may include aptamer or riboswitch sequences that can bind specific target molecules due to their three-dimensional structure.

In some embodiments, a trans-activating RNA (tracrRNA) is associated with crRNA to facilitate formation of a complex with Cas9 protein. In some embodiments, the tracrRNA sequence is about or more than about 5, 6, 7, 8, 9, 10, 11, 12, 13, 14, 15, 16, 17, 18, 19, 20, 25, 30, 40, 50, 60, 70, 80, 90, 100 or more nucleotides in length. In some embodiments, the tracrRNA is about 70 nucleotides in length.

In some embodiments, the tracrRNA and crRNA are contained in a single transcript called single guide RNA (sgRNA). In some embodiments, the sgRNA includes a loop between the tracrRNA and sgRNA.

In some embodiments, the loop forming sequences are 3, 4, 5 or more nucleotides in length. In some embodiments, the loop has the sequence GAAA, AAAG, CAAA, AAAC, UUUU, UUAUAU, UUA, UUU and/or AAUCA. In some embodiments, the loop has the sequence GAAA. In some embodiments, the loop has the sequence AAAG. In some embodiments, the loop has the sequence CAAA. In some embodiments, the loop has the sequence AAAC. In some embodiments, the loop has the sequence AAUCA. In some embodiments, the loop has the sequence UUUU. In some embodiments, the loop has the sequence UUAUAU. In some embodiments, the loop has the sequence UUA. In some embodiments, the loop has the sequence UUU. In some embodiments, the loop has the sequence AAUCA.

In some embodiments, the tracrRNA and crRNA form a hairpin loop. In some embodiments, sgRNA has at least two or more hairpins. In some embodiments, sgRNA has two, three, four or five hairpins.

In some embodiments, sgRNA includes a transcription termination sequence, which includes a polyT sequences comprising six nucleotides.

In some embodiments, the sgRNA comprises a sequence having at least 80% identity to 5′-GUUUUAGUACUCUGUAAUUUUGAAAAAAAUUACAGAAUCUACUAAAACAAGGC AAAAUGCCGUGUUUAUCUCGUCAACUUGUUGACGAGAUUUUUUU-3′ (SEQ ID NO: 43) for Seq2Cas9,

In some embodiments, the sgRNA comprises a sequence having at least 80% identity to 5′-GUUUUAGAGCUAUGUUGGAAACAACAUAGCGAGUUAAAAUAAGGCAUUGUCCG UUAUCAGCUUUUAAAGCAAGCACUGUCUCGGUGCUUUUUU-3′ (SEQ ID NO: 44) for EhiCas9,

In some embodiments, the sgRNA comprises a sequence having at least 80% identity to 5′-GUUUUUGUACUCUCAAGAUUUGAAAAAAUCUUGCAGAGCCUACAAAGAUAAGG CUUCAUGCCGAAUUCAAGCACCCCAUGUUUACAUGGGGUGCUUUU-3′ (SEQ ID NO: 45) for SeqCas9,

In some embodiments, the sgRNA comprises a sequence having at least 80% identity to 5′-GUUUUAGUACUCUGUAAUUUUGAAAAAAAUUACAGAAUCUACUAAAACAAGGC AAAAUGCCGUGUUUAUCUCGUCAACUUGUUUGGCGAGAUUUUUUUU-3′ (SEQ ID NO: 46) for SsiCas9,

In some embodiments, the sgRNA comprises a sequence having at least 80% identity to 5′-GUUUUUGUACUCUCAAGAUUUGAAAAAAUCUUGCAGAAGCUACAAAGAUAAGG CUUCAUGCCGAAAUCAACACCCUGUCUAUGACGGGGUGUUUU-3′ (SEQ ID NO: 47) for SinCas9,

In some embodiments, the sgRNA comprises a sequence having at least 80% identity to 5′-GUUUUAGAGCUGUGUUGUGAAAACAACACAGCAAGUUAAAAUAAGGCUUUGUC CGUACACAACUUGAAAAAGUGCGCACCGAUUCGGUGCUUUUUU-3′ (SEQ ID NO: 48) for SsaCas9,

In some embodiments, the sgRNA comprises a sequence having at least 80% identity to 5′-GUUUUUGUACUCUCAAGAUUUGAAAAAAUCUUGCAGAAGCUACAAAGAUAAGG CUUCAUGCCGAAAUCAACACCCUGUCAUUUUAUGGCAGGGUGUUUU-3′ (SEQ ID NO: 49) for Ssc2Cas9,

In some embodiments, the sgRNA comprises a sequence having at least 80% identity to 5′-GUUUUUGUACUCUCAAGAUUUGAAAAAAUCUUGCAGAAGCUACAAAGAUAAGG CUUCAUGCCGAAUUCAACACCCUGUCAUUUAUGGCGGGGUGUUUU-3′ (SEQ ID NO: 50) for Sor2Cas9,

In some embodiments, the sgRNA comprises a sequence having at least 80% identity to 5′-GUUUUUGUACUCUCAAGAUUUGAAAAAAUCUUGUAGAAGCUACAAAGAUAAGG CUUCAUGCCGAAUUCAACACCCUGUCAUUUAUGGCGGGGUGUUUCGUUUU-3′ (SEQ ID NO: 51) for SorCas9,

In some embodiments, the sgRNA comprises a sequence having at least 80% identity to 5′-GUUUUAGUACUCUGUAAUUUUGAAAAAAAUUACAGAAUCUACUGAAACAAGAC UAUAUGUCGUGUUUAUCCCACUAAUUUAUUAGUGGGAUUUUUU-3′ (SEQ ID NO: 52) for SwaCas9,

In some embodiments, the sgRNA comprises a sequence having at least 80% identity to 5′-GUUUUAGUACUCUGUAAUUUUGAAAAAAAUUACAGAAUCUACUAAAACAAGGC AAAAUGCCGUGUUUAUCUCGUCAACUUGUUGACGAGAUUUUUUU-3′ (SEQ ID NO: 53) for SscCas9,

In some embodiments, the sgRNA comprises a sequence having at least 80% identity to 5′-GUUUUUGUACUCUCAAGAUUUGAAAAAAUCUUGCUGAGCCUACAAAGAUAAGG 15 CUUUAUGCCGAAUUCAAGCACCCCAUGUUUUGACAUGGGGUGCUUUU-3′ (SEQ ID NO: 54) for SgaCas9,

In some embodiments, the sgRNA comprises a sequence having at least 80% identity to 5′-GUUUUAGAUGAUUGUUAGAUCGAAAGAUCUAACAACCAGAUUUUAAAAUCAAA CAAUGUAUCUUUGAUACUAAGUUUCAACGCGGUAUUAUUACCGUCCUGCCUCA GCUCUAUAGCGGAGGUUUUUU-3′ (SEQ ID NO: 55) for LkuCas9, and

In some embodiments, the sgRNA comprises a sequence having at least 80% identity to 5′-GUUUUUGUACUCUCAAGAUUUGAAAAAAUCUUGCAGAGCCUACAAAGAUAAG GCUUUAUGCCGAAAUCAAGCACCCCGUUUCGUACGGGGUGCUUUU-3′ (SEQ ID NO: 56) for SsuCas9.

For SEQ ID NOs 43-56: Direct repeat (italics and underlined), tetraloop (italics), tracrRNA (underlined)

The guide RNA is added to the 5′ end of the Cas9. In some embodiments, the sgRNA comprises a sequence having 80%, 81%, 82%, 83%, 84%, 85%, 86%, 87%, 88%, 89%, 90%, 91%, 92%, 93%, 94%, 95%, 96%, 97%, 98%, 99% or more identity to SEQ ID NO: 43, 44, 45, 46, 47, 48, 49, 50, 51, 52, 53, 54, 55 or 56. In some embodiments, the sgRNA comprises a sequence identical to SEQ ID NO: 43, 44, 45, 46, 47, 48, 49, 50, 51, 52, 53, 54, 55 or 56.

In some embodiments, the tracrRNA is a separate transcript, not contained with crRNA sequence in the same transcript.

### Cas9 Fusion Proteins

In some embodiments, the Cas9 enzyme is fused to one or more heterologous protein domains. In some embodiments, the Cas9 enzyme is fused to more than about 1, 2, 3, 4, 5, 6, 7, 8, 9, 10 or more protein domains. In some embodiments, the heterologous protein domain is fused to the C-terminus of the Cas9 enzyme. In some embodiments, the heterologous protein domain is fused to the N-terminus of the Cas9 enzyme. In some embodiments, the heterologous protein domain is fused internally, between the C-terminus and the N-terminus of the Cas9 enzyme. In some embodiments, the internal fusion is made within the Cas9 RuvCI, RuvC II, RuvCIII, HNH, REC I, or PAM interacting domain.

A Cas9 protein may be directly or indirectly linked to another protein domain. In some embodiments, a suitable CRISPR system contains a linker or spacer that joins a Cas9 protein and a heterologous protein. An amino acid linker or spacer is generally designed to be flexible or to interpose a structure, such as an alpha-helix, between the two protein moieties. A linker or spacer can be relatively short, or can be longer. Typically, a linker or spacer contains for example 1-100 (e.g., 1-100, 5-100, 10-100, 20-100 30-100, 40-100, 50-100, 60-100, 70-100, 80-100, 90-100, 5-55, 10-50, 10-45, 10-40, 10-35, 10-30, 10-25, 10-20) amino acids in length. In some embodiments, a linker or spacer is equal to or longer than 1, 2, 3, 4, 5, 6, 7, 8, 9, 10, 15, 20, 25, 30, 35, 40, 45, 50, 55, 60, 65, 70, 75, 80, 85, 90, 95, or 100 amino acids in length. Typically, a longer linker may decrease steric hindrance. In some embodiments, a linker will comprise a mixture of glycine and serine residues. In some embodiments, the linker may additionally comprise threonine, proline and/or alanine residues.

In some embodiments, a Cas9 protein is fused to cellular localization signals, epitope tags, reporter genes, and protein domains with enzymatic activity, epigenetic modifying activity, RNA cleavage activity, nucleic acid binding activity, transcription modulation activity. In some embodiments, the Cas9 protein is fused to a nuclear localization sequence (NLS), a FLAG tag, a HIS tag, and/or a HA tag.

Suitable fusion partners include, but are not limited to, a polypeptide that provides for methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, demyristoylation activity, integrase activity, transposase activity, recombinase activity, polymerase activity, ligase activity, helicase activity, or nuclease activity, any of which can modify DNA or a DNA-associated polypeptide (e.g., a histone or DNA binding protein). In some embodiments, the Cas9 protein is fused to a histone demethylase, a transcriptional activator or a deaminase.

Further suitable fusion partners include, but are not limited to boundary elements (e.g., CTCF), proteins and fragments thereof that provide periphery recruitment (e.g., Lamin A, Lamin B, etc.), and protein docking elements (e.g., FKBP/FRB, Pill/Abyl, etc.).

In particular embodiments, a Cas9 is fused to a cytidine or adenosine deaminase domain, e.g., for use in base editing. In some embodiments, the terms “cytidine deaminase” and “cytosine deaminase” can be used interchangeably. In certain embodiments, the cytidine deaminase domain may have sequence identity of 70%, 75%, 80%, 85%, 90%, 91%, 92%, 93%, 94%, 95%, 96%, 97%, 98%, 99% or more to any cytidine deaminase described herein. In some embodiments, the cytidine deaminase domain has cytidine deaminase activity, (e.g., converting C to U). In certain embodiments, the adenosine deaminase domain may have sequence identity of 70%, 75%, 80%, 85%, 90%, 91%, 92%, 93%, 94%, 95%, 96%, 97%, 98%, 99% or more to any adenosine deaminase described herein. In some embodiments, the adenosine deaminase domain has adenosine deaminase activity, (e.g., converting A to I). In some embodiments, the terms “adenosine deaminase” and “adenine deaminase” can be used interchangeably.

In some embodiments, a cytidine deaminase can comprise all or a portion of an apolipoprotein B mRNA editing complex (APOBEC) family deaminase. APOBEC is a family of evolutionarily conserved cytidine deaminases. Members of this family are C-to-U editing enzymes. The N-terminal domain of APOBEC like proteins is the catalytic domain, while the C-terminal domain is a pseudocatalytic domain. More specifically, the catalytic domain is a zinc dependent cytidine deaminase domain and is important for cytidine deamination. APOBEC family members include APOBECI, APOBEC2, APOBEC3A, APOBEC3B, APOBEC3C, APOBEC3D (“APOBEC3E” now refers to this), APOBEC3F, APOBEC3G, APOBEC3H, APOBEC4, and Activation-induced (cytidine or cytosine) deaminase. In some embodiments, a deaminase incorporated into a fusion protein comprises all or a portion of an APOBECI deaminase. In some embodiments, a deaminase incorporated into a fusion protein comprises all or a portion of APOBEC2 deaminase. In some embodiments, a deaminase incorporated into a fusion protein comprises all or a portion of is an APOBEC3 deaminase. In some embodiments, a deaminase incorporated into a fusion protein comprises all or a portion of an APOBEC3A deaminase. In some embodiments, a deaminase incorporated into a fusion protein comprises all or a portion of APOBEC3B deaminase. In some embodiments, a deaminase incorporated into a fusion protein comprises all or a portion of APOBEC3C deaminase. In some embodiments, a deaminase incorporated into a fusion protein comprises all or a portion of APOBEC3D deaminase. In some embodiments, a deaminase incorporated into a fusion protein comprises all or a portion of APOBEC3E deaminase. In some embodiments, a deaminase incorporated into a fusion protein comprises all or a portion of APOBEC3F deaminase. In some embodiments, a deaminase incorporated into a fusion protein comprises all or a portion of APOBEC3G deaminase. In some embodiments, a deaminase incorporated into a fusion protein comprises all or a portion of APOBEC3H deaminase. In some embodiments, a deaminase incorporated into a fusion protein comprises all or a portion of APOBEC4 deaminase. In some embodiments, a deaminase incorporated into a fusion protein comprises all or a portion of activation-induced deaminase (AID). In some embodiments a deaminase incorporated into a fusion protein comprises all or a portion of cytidine deaminase 1 (CDA1). It should be appreciated that a fusion protein can comprise a deaminase from any suitable organism (e.g., a human or a rat). In some embodiments, a deaminase domain of a fusion protein is from a human, chimpanzee, gorilla, monkey, cow, dog, rat, or mouse. In some embodiments, the deaminase domain of the fusion protein is derived from rat (e.g., rat APOBECI). In some embodiments, the deaminase domain is human APOBEC1. In some embodiments, the deaminase domain is pmCDA1. Sequences of exemplary cytidine deaminases are provided below.

pmCDA1 (Petromyzon marinus)
(SEQ ID NO: 57)
MTDAEYVRIHEKLDIYTFKKQFFNNKKSVSHRCYVLFELKRRGERRACFW
GYAVNKPQSGTERGIHAEIFSIRKVEEYLRDNPGQFTINWYSSWSPCADC
AEKILEWYNQELRGNGHTLKIWACKLYYEKNARNQIGLWNLRDNGVGLNV
MVSEHYQCCRKIFIQSSHNQLNENRWLEKTLKRAEKRRSELSIMIQVKIL
HTTKSPAV
Human AID:
(SEQ ID NO: 58)
MDSLLMNRRKFLYQFKNVRWAKGRRETYLCYVVKRRDSATSFSLDFGYLR
NKNGCHVELLFLRYISDWDLDPGRCYRVTWFTSWSPCYDCARHVADFLRG
NPNLSLRIFTARLYFCEDRKAEPEGLRRLHRAGVQIAIMTFKAPV
Human AID:
(SEQ ID NO: 59)
MDSLLMNRRKFLYQFKNVRWAKGRRETYLCYVVKRRDSATSFSLDFGYLR
NKNGCHVELLFLRYISDWDLDPGRCYRVTWFTSWSPCYDCARHVADFLRG
NPNLSLRIFTARLYFCEDRKAEPEGLRRLHRAGVQIAIMTFKDYFYCWNT
FVENHERTFKAWEGLHENSVRLSRQLRRILLPLYEVDDLRDAFRTLGL
(underline: nuclear localization sequence; double
underline: nuclear export signal)
Mouse AID:
(SEQ ID NO: 60)
MDSLLMKQKKFLYHFKNVRWAKGRHETYLCYVVKRRDSATSCSLDFGHLR
NKSGCHVELLFLRYISDWDLDPGRCYRVTWFTSWSPCYDCARHVAEFLRW
NPNLSLRIFTARLYFCEDRKAEPEGLRRLHRAGVQIGIMTFKDYFYCWNT
FVENRERTFKAWEGLHENSVRLTRQLRRILLPLYEVDDLRDAFRMLGE
(underline: nuclear localization sequence; double
underline: nuclear export signal)
Canine AID:
(SEQ ID NO: 61)
MDSLLMKQRKFLYHFKNVRWAKGRHETYLCYVVKRRDSATSFSLDFGHLR
NKSGCHVELLFLRYISDWDLDPGRCYRVTWFTSWSPCYDCARHVADFLRG
YPNLSLRIFAARLYFCEDRKAEPEGLRRLHRAGVQIAIMTFKDYFYCWNT
FVENREKTFKAWEGLHENSVRLSRQLRRILLPLYEVDDLRDAFRTLGL
(underline: nuclear localization sequence; double
underline: nuclear export signal)
Bovine AID:
(SEQ ID NO: 62)
MDSLLKKQRQFLYQFKNVRWAKGRHETYLCYVVKRRDSPTSFSLDFGHLR
NKAGCHVELLFLRYISDWDLDPGRCYRVTWFTSWSPCYDCARHVADFLRG
YPNLSLRIFTARLYFCDKERKAEPEGLRRLHRAGVQIAIMTFKDYFYCWN
TFVENHERTFKAWEGLHENSVRLSRQLRRILLPLYEVDDLRDAFRTLGL
(underline: nuclear localization sequence; double
underline: nuclear export signal)
Rat AID:
(SEQ ID NO: 63)
MAVGSKPKAALVGPHWERERIWCFLCSTGLGTQQTGQTSRWLRPAATQDP
VSPPRSLLMKQRKFLYHFKNVRWAKGRHETYLCYVVKRRDSATSFSLDFG
YLRNKSGCHVELLFLRYISDWDLDPGRCYRVTWFTSWSPCYDCARHVADF
LRGNPNLSLRIFTARLTGWGALPAGLMSPARPSDYFYCWNTFVENHERTF
KAWEGLHENSVRLSRRLRRILLPLYEVDDLRDAFRTLGL
(underline: nuclear localization sequence; double
underline: nuclear export signal)
clAID (Canis lupus familiaris):
(SEQ ID NO: 64)
MDSLLMKQRKFLYHFKNVRWAKGRHETYLCYVVKRRDSATSFSLDFGHLR
NKSGCHVELLFLRYISDWDLDPGRCYRVTWFTSWSPCYDCARHVADFLRG
YPNLSLRIFAARLYFCEDRKAEPEGLRRLHRAGVQIAIMTFKDYFYCWNT
FVENREKTFKAWEGLHENSVRLSRQLRRILLPLYEVDDLRDAFRTLGL
btAID (Bos taurus):
(SEQ ID NO: 65)
MDSLLKKQRQFLYQFKNVRWAKGRHETYLCYVVKRRDSPTSFSLDFGHLR
NKAGCHVELLFLRYISDWDLDPGRCYRVTWFTSWSPCYDCARHVADFLRG
YPNLSLRIFTARLYFCDKERKAEPEGLRRLHRAGVQIAIMTFKDYFYCWN
TFVENHERTFKAWEGLHENSVRLSRQLRRILLPLYEVDDLRDAFRTLGL
mAID (Mus musculus):
(SEQ ID NO: 66)
MDSLLMNRRKFLYQFKNVRWAKGRRETYLCYVVKRRDSATSFSLDFGYLR
NKNGCHVELLFLRYISDWDLDPGRCYRVTWFTSWSPCYDCARHVADFLRG
NPNLSLRIFTARLYFCEDRKAEPEGLRRLHRAGVQIAIMTFKDYFYCWNT
FVENHERTFKAWEGLHENSVRLSRQLRRILLPLYEVDDLRDAFRTLGL
rAPOBEC-1 (Rattus norvegicus):
(SEQ ID NO: 67)
MSSETGPVAVDPTLRRRIEPHEFEVFFDPRELRKETCLLYEINWGGRHSI
WRHTSQNTNKHVEVNFIEKFTTERYFCPNTRCSITWFLSWSPCGECSRAI
TEFLSRYPHVTLFIYIARLYHHADPRNRQGLRDLISSGVTIQIMTEQESG
YCWRNFVNYSPSNEAHWPRYPHLWVRLYVLELYCIILGLPPCLNILRRKQ
PQLTFFTIALQSCHYQRLPPHILWATGLK
maAPOBEC-1 (Mesocricetus auratus):
(SEQ ID NO: 68)
MSSETGPVVVDPTLRRRIEPHEFDAFFDQGELRKETCLLYEIRWGGRHNI
WRHTGQNTSRHVEINFIEKFTSERYFYPSTRCSIVWFLSWSPCGECSKAI
TEFLSGHPNVTLFIYAARLYHHTDQRNRQGLRDLISRGVTIRIMTEQEYC
YCWRNFVNYPPSNEVYWPRYPNLWMRLYALELYCIHLGLPPCLKIKRRHQ
YPLTFFRLNLQSCHYQRIPPHILWATGFI
ppAPOBEC-1 (Pongo pygmaeus):
(SEQ ID NO: 69)
MTSEKGPSTGDPTLRRRIESWEFDVFYDPRELRKETCLLYEIKWGMSRKI
WRSSGKNTTNHVEVNFIKKFTSERRFHSSISCSITWFLSWSPCWECSQAI
REFLSQHPGVTLVIYVARLFWHMDQRNRQGLRDLVNSGVTIQIMRASEYY
HCWRNFVNYPPGDEAHWPQYPPLWMMLYALELHCIILSLPPCLKISRRWQ
NHLAFFRLHLQNCHYQTIPPHILLATGLIHPSVTWR
ocAPOBEC1 (Oryctolagus cuniculus):
(SEQ ID NO: 70)
MASEKGPSNKDYTLRRRIEPWEFEVFFDPQELRKEACLLYEIKWGASSKT
WRSSGKNTTNHVEVNFLEKLTSEGRLGPSTCCSITWFLSWSPCWECSMAI
REFLSQHPGVTLIIFVARLFQHMDRRNRQGLKDLVTSGVTVRVMSVSEYC
YCWENFVNYPPGKAAQWPRYPPRWMLMYALELYCIILGLPPCLKISRRHQ
KQLTFFSLTPQYCHYKMIPPYILLATGLLQPSVPWR
mdAPOBEC-1 (Monodelphis domestica):
(SEQ ID NO: 71)
MNSKTGPSVGDATLRRRIKPWEFVAFFNPQELRKETCLLYEIKWGNQNIW
RHSNQNTSQHAEINFMEKFTAERHFNSSVRCSITWFLSWSPCWECSKAIR
KFLDHYPNVTLAIFISRLYWHMDQQHRQGLKELVHSGVTIQIMSYSEYHY
CWRNFVDYPQGEEDYWPKYPYLWIMLYVLELHCIILGLPPCLKISGSHSN
QLALFSLDLQDCHYQKIPYNVLVATGLVQPFVTWR
ppAPOBEC-2 (Pongo pygmaeus):
(SEQ ID NO: 72)
MAQKEEAAAATEAASQNGEDLENLDDPEKLKELIELPPFEIVTGERLPAN
FFKFQFRNVEYSSGRNKTFLCYVVEAQGKGGQVQASRGYLEDEHAAAHAE
EAFFNTILPAFDPALRYNVTWYVSSSPCAACADRIIKTLSKTKNLRLLIL
VGRLFMWEELEIQDALKKLKEAGCKLRIMKPQDFEYVWQNFVEQEEGESK
AFQPWEDIQENFLYYEEKLADILK
btAPOBEC-2 (Bos taurus):
(SEQ ID NO: 73)
MAQKEEAAAAAEPASQNGEEVENLEDPEKLKELIELPPFEIVTGERLPAH
YFKFQFRNVEYSSGRNKTFLCYVVEAQSKGGQVQASRGYLEDEHATNHAE
EAFFNSIMPTFDPALRYMVTWYVSSSPCAACADRIVKTLNKTKNLRLLIL
VGRLFMWEEPEIQAALRKLKEAGCRLRIMKPQDFEYIWQNFVEQEEGESK
AFEPWEDIQENFLYYEEKLADILK
mAPOBEC-3-(1) (Mus musculus):
(SEQ ID NO: 74)
MQPQRLGPRAGMGPFCLGCSHRKCYSPIRNLISQETFKFHFKNLGYAKGR
KDTFLCYEVTRKDCDSPVSLHHGVFKNKDNIHAEICFLYWFHDKVLKVLS
PREEFKITWYMSWSPCFECAEQIVRFLATHHNLSLDIFSSRLYNVQDPET
QQNLCRLVQEGAQVAAMDLYEFKKCWKKFVDNGGRRFRPWKRLLTNFRYQ
DSKLQEILRPCYISVPSSSSSTLSNICLTKGLPETRFWVEGRRMDPLSEE
EFYSQFYNQRVKHLCYYHRMKPYLCYQLEQFNGQAPLKGCLLSEKGKQHA
EILFLDKIRSMELSQVTITCYLTWSPCPNCAWQLAAFKRDRPDLILHIYT
SRLYFHWKRPFQKGLCSLWQSGILVDVMDLPQFTDCWTNFVNPKRPFWPW
KGLEIISRRTQRRLRRIKESWGLQDLVNDFGNLQLGPPMS
Mouse APOBEC-3-(2):
(SEQ ID NO: 75)
MGPFCLGCSHRKCYSPIRNLISQETFKFHFKNLGYAKGRKDTFLCYEVTR
KDCDSPVSLHHGVFKNKDNIHAEICFLYWFHDKVLKVLSPREEFKITWYM
SWSPCFECAEQIVRFLATHHNLSLDIFSSRLYNVQDPETQQNLCRLVQEG
AQVAAMDLYEFKKCWKKFVDNGGRRFRPWKRLLTNFRYQDSKLQEILRPC
YIPVPSSSSSTLSNICLTKGLPETRFCVEGRRMDPLSEEEFYSQFYNQRV
KHLCYYHRMKPYLCYQLEQFNGQAPLKGCLLSEKGKQHAEILFLDKIRSM
ELSQVTITCYLTWSPCPNCAWQLAAFKRDRPDLILHIYTSRLYFHWKRPF
QKGLCSLWQSGILVDVMDLPQFTDCWTNFVNPKRPFWPWKGLEIISRRTQ
RRLRRIKESWGLQDLVNDFGNLQLGPPMS (italic: nucleic
acid editing domain)
Rat APOBEC-3:
(SEQ ID NO: 76)
MGPFCLGCSHRKCYSPIRNLISQETFKFHFKNRLRYAIDRKDTFLCYEVT
RKDCDSPVSLHHGVFKNKDNIHAEICFLYWFHDKVLKVLSPREEFKITWY
MSWSPCFECAEQVLRFLATHHNLSLDIFSSRLYNIRDPENQQNLCRLVQE
GAQVAAMDLYEFKKCWKKFVDNGGRRFRPWKKLLTNFRYQDSKLQEILRP
CYIPVPSSSSSTLSNICLTKGLPETRFCVERRRVHLLSEEEFYSQFYNQR
VKHLCYYHGVKPYLCYQLEQFNGQAPLKGCLLSEKGKQHAEILFLDKIRS
MELSQVIITCYLTWSPCPNCAWQLAAFKRDRPDLILHIYTSRLYFHWKRP
FQKGLCSLWQSGILVDVMDLPQFTDCWTNFVNPKRPFWPWKGLEIISRRT
QRRLHRIKESWGLQDLVNDFGNLQLGPPMS (italic: nucleic
acid editing domain)
hAPOBEC-3A (Homo sapiens):
(SEQ ID NO: 77)
MEASPASGPRHLMDPHIFTSNFNNGIGRHKTYLCYEVERLDNGTSVKMDQ
HRGFLHNQAKNLLCGFYGRHAELRFLDLVPSLQLDPAQIYRVTWFISWSP
CFSWGCAGEVRAFLQENTHVRLRIFAARIYDYDPLYKEALQMLRDAGAQV
SIMTYDEFKHCWDTFVDHQGCPFQPWDGLDEHSQALSGRLRAILQNQGN
hAPOBEC-3F (Homo sapiens)
(SEQ ID NO: 78)
MKPHFRNTVERMYRDTFSYNFYNRPILSRRNTVWLCYEVKTKGPSRPRLD
AKIFRGQVYSQPEHHAEMCFLSWFCGNQLPAYKCFQITWFVSWTPCPDCV
AKLAEFLAEHPNVTLTISAARLYYYWERDYRRALCRLSQAGARVKIMDDE
EFAYCWENFVYSEGQPFMPWYKFDDNYAFLHRTLKEILRNPMEAMYPHIF
YFHFKNLRKAYGRNESWLCFTMEVVKHHSPVSWKRGVFRNQVDPETHCHA
ERCFLSWFCDDILSPNTNYEVTWYTSWSPCPECAGEVAEFLARHSNVNLT
IFTARLYYFWDTDYQEGLRSLSQEGASVEIMGYKDFKYCWENFVYNDDEP
FKPWKGLKYNFLFLDSKLQEILE
Rhesus macaque APOBEC-3G:
(SEQ ID NO: 79)
MVEPMDPRTFVSNFNNRPILSGLNTVWLCCEVKTKDPSGPPLDAKIFQGK
VYSKAKYHPEMRFLRWFHKWRQLHHDQEYKVTWYVSWSPCTRCANSVATF
LAKDPKVTLTIFVARLYYFWKPDYQQALRILCQKRGGPHATMKIMNYNEF
QDCWNKFVDGRGKPFKPRNNLPKHYTLLQATLGELLRHLMDPGTFTSNFN
NKPWVSGQHETYLCYKVERLHNDTWVPLNQHRGFLRNQAPNIHGFPKGRH
AELCFLDLIPFWKLDGQQYRVTCFTSWSPCFSCAQEMAKFISNNEHVSLC
IFAARIYDDQGRYQEGLRALHRDGAKIAMMNYSEFEYCWDTFVDRQGRPF
QPWDGLDEHSQALSGRLRAI (italic: nucleic acid editing
domain; underline: cytoplasmic localization
signal)
Chimpanzee APOBEC-3G:
(SEQ ID NO: 80)
MKPHFRNPVERMYQDTESDNFYNRPILSHRNTVWLCYEVKTKGPSRPPLD
AKIFRGQVYSKLKYHPEMRFFHWFSKWRKLHRDQEYEVTWYISWSPCTKC
TRDVATFLAEDPKVTLTIFVARLYYFWDPDYQEALRSLCQKRDGPRATMK
IMNYDEFQHCWSKFVYSQRELFEPWNNLPKYYILLHIMLGEILRHSMDPP
TFTSNFNNELWVRGRHETYLCYEVERLHNDTWVLLNQRRGFLCNQAPHKH
GFLEGRHAELCFLDVIPFWKLDLHQDYRVTCFTSWSPCFSCAQEMAKFIS
NNKHVSLCIFAARIYDDQGRCQEGLRTLAKAGAKISIMTYSEFKHCWDTF
VDHQGCPFQPWDGLEEHSQALSGRLRAILQNQGN (italic:
nucleic acid editing domain; underline:
cytoplasmic localization signal)
Green monkey APOBEC-3G:
(SEQ ID NO: 81)
MNPQIRNMVEQMEPDIFVYYFNNRPILSGRNTVWLCYEVKTKDPSGPPLD
ANIFQGKLYPEAKDHPEMKFLHWFRKWRQLHRDQEYEVTWYVSWSPCTRC
ANSVATFLAEDPKVTLTIFVARLYYFWKPDYQQALRILCQERGGPHATMK
IMNYNEFQHCWNEFVDGQGKPFKPRKNLPKHYTLLHATLGELLRHVMDPG
TFTSNFNNKPWVSGQRETYLCYKVERSHNDTWVLLNQHRGFLRNQAPDRH
GFPKGRHAELCFLDLIPFWKLDDQQYRVTCFTSWSPCFSCAQKMAKFISN
NKHVSLCIFAARIYDDQGRCQEGLRTLHRDGAKIAVMNYSEFEYCWDTFV
DRQGRPFQPWDGLDEHSQALSGRLRAI (italic: nucleic acid
editing domain; underline: cytoplasmic
localization signal)
Human APOBEC-3G:
(SEQ ID NO: 82)
MKPHFRNTVERMYRDTFSYNFYNRPILSRRNTVWLCYEVKTKGPSRPPLD
AKIFRGQVYSELKYHPEMRFFHWFSKWRKLHRDQEYEVTWYISWSPCTKC
TRDMATFLAEDPKVTLTIFVARLYYFWDPDYQEALRSLCQKRDGPRATMK
IMNYDEFQHCWSKFVYSQRELFEPWNNLPKYYILLHIMLGEILRHSMDPP
TFTFNFNNEPWVRGRHETYLCYEVERMHNDTWVLLNQRRGFLCNQAPHKH
GFLEGRHAELCFLDVIPFWKLDLDQDYRVTCFTSWSPCFSCAQEMAKFIS
KNKHVSLCIFTARIYDDQGRCQEGLRTLAEAGAKISIMTYSEFKHCWDTF
VDHQGCPFQPWDGLDEHSQDLSGRLRAILQNQEN (italic:
nucleic acid editing domain; underline:
cytoplasmic localization signal)
Human APOBEC-3F:
(SEQ ID NO: 83)
MKPHFRNTVERMYRDTFSYNFYNRPILSRRNTVWLCYEVKTKGPSRPRLD
AKIFRGQVYSQPEHHAEMCFLSWFCGNQLPAYKCFQITWFVSWTPCPDCV
AKLAEFLAEHPNVTLTISAARLYYYWERDYRRALCRLSQAGARVKIMDDE
EFAYCWENFVYSEGQPFMPWYKFDDNYAFLHRTLKEILRNPMEAMYPHIF
YFHFKNLRKAYGRNESWLCFTMEVVKHHSPVSWKRGVFRNQVDPETHCHA
ERCFLSWFCDDILSPNTNYEVTWYTSWSPCPECAGEVAEFLARHSNVNLT
IFTARLYYFWDTDYQEGLRSLSQEGASVEIMGYKDFKYCWENFVYNDDEP
FKPWKGLKYNFLFLDSKLQEILE (italic: nucleic acid
editing domain)
Human APOBEC-3B:
(SEQ ID NO: 84)
MNPQIRNPMERMYRDTFYDNFENEPILYGRSYTWLCYEVKIKRGRSNLLW
DTGVFRGQVYFKPQYHAEMCFLSWFCGNQLPAYKCFQITWFVSWTPCPDC
VAKLAEFLSEHPNVTLTISAARLYYYWERDYRRALCRLSQAGARVTIMDY
EEFAYCWENFVYNEGQQFMPWYKFDENYAFLHRTLKEILRYLMDPDTFTF
NFNNDPLVLRRRQTYLCYEVERLDNGTWVLMDQHMGFLCNEAKNLLCGFY
GRHAELRFLDLVPSLQLDPAQIYRVTWFISWSPCFSWGCAGEVRAFLQEN
THVRLRIFAARIYDYDPLYKEALQMLRDAGAQVSIMTYDEFEYCWDTFVY
RQGCPFQPWDGLEEHSQALSGRLRAILQNQGN (italic: nucleic
acid editing domain)
Rat APOBEC-3B:
(SEQ ID NO: 85)
MQPQGLGPNAGMGPVCLGCSHRRPYSPIRNPLKKLYQQTFYFHFKNVRYA
WGRKNNFLCYEVNGMDCALPVPLRQGVFRKQGHIHAELCFIYWFHDKVLR
VLSPMEEFKVTWYMSWSPCSKCAEQVARFLAAHRNLSLAIFSSRLYYYLR
NPNYQQKLCRLIQEGVHVAAMDLPEFKKCWNKFVDNDGQPFRPWMRLRIN
FSFYDCKLQEIFSRMNLLREDVFYLQFNNSHRVKPVQNRYYRRKSYLCYQ
LERANGQEPLKGYLLYKKGEQHVEILFLEKMRSMELSQVRITCYLTWSPC
PNCARQLAAFKKDHPDLILRIYTSRLYFWRKKFQKGLCTLWRSGIHVDVM
DLPQFADCWTNFVNPQRPFRPWNELEKNSWRIQRRLRRIKESWGL
Bovine APOBEC-3B:
(SEQ ID NO: 86)
MDGWEVAFRSGTVLKAGVLGVSMTEGWAGSGHPGQGACVWTPGTRNTMNL
LREVLFKQQFGNQPRVPAPYYRRKTYLCYQLKQRNDLTLDRGCFRNKKQR
HAERFIDKINSLDLNPSQSYKIICYITWSPCPNCANELVNFITRNNHLKL
EIFASRLYFHWIKSFKMGLQDLQNAGISVAVMTHTEFEDCWEQFVDNQSR
PFQPWDKLEQYSASIRRRLQRILTAPI
Chimpanzee APOBEC-3B:
(SEQ ID NO: 87)
MNPQIRNPMEWMYQRTFYYNFENEPILYGRSYTWLCYEVKIRRGHSNLLW
DTGVFRGQMYSQPEHHAEMCFLSWFCGNQLSAYKCFQITWFVSWTPCPDC
VAKLAKFLAEHPNVTLTISAARLYYYWERDYRRALCRLSQAGARVKIMDD
EEFAYCWENFVYNEGQPFMPWYKFDDNYAFLHRTLKEIIRHLMDPDTFTF
NFNNDPLVLRRHQTYLCYEVERLDNGTWVLMDQHMGFLCNEAKNLLCGFY
GRHAELRFLDLVPSLQLDPAQIYRVTWFISWSPCFSWGCAGQVRAFLQEN
THVRLRIFAARIYDYDPLYKEALQMLRDAGAQVSIMTYDEFEYCWDTFVY
RQGCPFQPWDGLEEHSQALSGRLRAILQVRASSLCMVPHRPPPPPQSPGP
CLPLCSEPPLGSLLPTGRPAPSLPFLLTASFSFPPPASLPPLPSLSLSPG
HLPVPSFHSLTSCSIQPPCSSRIRETEGWASVSKEGRDLG
Human APOBEC-3C:
(SEQ ID NO: 88)
MNPQIRNPMKAMYPGTFYFQFKNLWEANDRNETWLCFTVEGIKRRSVVSW
KTGVFRNQVDSETHCHAERCFLSWFCDDILSPNTKYQVTWYTSWSPCPDC
AGEVAEFLARHSNVNLTIFTARLYYFQYPCYQEGLRSLSQEGVAVEIMDY
EDFKYCWENFVYNDNEPFKPWKGLKTNFRLLKRRLRESLQ (italic:
nucleic acid editing domain)
Gorilla APOBEC-3C
(SEQ ID NO: 89)
MNPQIRNPMKAMYPGTFYFQFKNLWEANDRNETWLCFTVEGIKRRSVVSW
KTGVFRNQVDSETHCHAERCFLSWECDDILSPNTNYQVTWYTSWSPCPEC
AGEVAEFLARHSNVNLTIFTARLYYFQDTDYQEGLRSLSQEGVAVKIMDY
KDFKYCWENFVYNDDEPFKPWKGLKYNFRFLKRRLQEILE (italic:
nucleic acid editing domain)
Human APOBEC-3A:
(SEQ ID NO: 90)
MEASPASGPRHLMDPHIFTSNFNNGIGRHKTYLCYEVERLDNGTSVKMDQ
HRGFLHNQAKNLLCGFYGRHAELRFLDLVPSLQLDPAQIYRVTWFISWSP
CFSWGCAGEVRAFLQENTHVRLRIFAARIYDYDPLYKEALQMLRDAGAQV
SIMTYDEFKHCWDTFVDHQGCPFQPWDGLDEHSQALSGRLRAILQNQGN
(italic: nucleic acid editing domain)
Rhesus macaque APOBEC-3A:
(SEQ ID NO: 91)
MDGSPASRPRHLMDPNTFTFNFNNDLSVRGRHQTYLCYEVERLDNGTWV
PMDERRGFLCNKAKNVPCGDYGCHVELRFLCEVPSWQLDPAQTYRVTWF
ISWSPCFRRGCAGQVRVFLQENKHVRLRIFAARIYDYDPLYQEALRTLR
DAGAQVSIMTYEEFKHCWDTFVDRQGRPFQPWDGLDEHSQALSGRLRAI
LQNQGN (italic: nucleic acid editing domain)
Bovine APOBEC-3A:
(SEQ ID NO: 92)
MDEYTFTENFNNQGWPSKTYLCYEMERLDGDATIPLDEYKGFVRNKGLD
QPEKPCHAELYFLGKIHSWNLDRNQHYRLTCFISWSPCYDCAQKLTTFL
KENHHISLHILASRIYTHNRFGCHQSGLCELQAAGARITIMTFEDFKHC
WETFVDHKGKPFQPWEGLNVKSQALCTELQAILKTQQN (italic:
nucleic acid editing domain)
Human APOBEC-3H:
(SEQ ID NO: 93)
MALLTAETFRLQFNNKRRLRRPYYPRKALLCYQLTPQNGSTPTRGYFEN
KKKCHAEICFINEIKSMGLDETQCYQVTCYLTWSPCSSCAWELVDFIKA
HDHLNLGIFASRLYYHWCKPQQKGLRLLCGSQVPVEVMGFPKFADCWEN
FVDHEKPLSFNPYKMLEELDKNSRAIKRRLERIKIPGVRAQGRYMDILC
DAEV (italic: nucleic acid editing domain)
Rhesus macaque APOBEC-3H:
(SEQ ID NO: 94)
MALLTAKTFSLQFNNKRRVNKPYYPRKALLCYQLTPQNGSTPTRGHLKN
KKKDHAEIRFINKIKSMGLDETQCYQVTCYLTWSPCPSCAGELVDFIKA
HRHLNLRIFASRLYYHWRPNYQEGLLLLCGSQVPVEVMGLPEFTDCWEN
FVDHKEPPSFNPSEKLEELDKNSQAIKRRLERIKSRSVDVLENGLRSLQ
LGPVTPSSSIRNSR
Human APOBEC-3D:
(SEQ ID NO: 95)
MNPQIRNPMERMYRDTFYDNFENEPILYGRSYTWLCYEVKIKRGRSNLL
WDTGVFRGPVLPKRQSNHRQEVYFRFENHAEMCFLSWFCGNRLPANRRF
QITWFVSWNPCLPCVVKVTKFLAEHPNVTLTISAARLYYYRDRDWRWVL
LRLHKAGARVKIMDYEDFAYCWENFVCNEGQPFMPWYKFDDNYASLHRT
LKEILRNPMEAMYPHIFYFHFKNLLKACGRNESWLCFTMEVTKHHSAVF
RKRGVFRNQVDPETHCHAERCFLSWFCDDILSPNTNYEVTWYTSWSPCP
ECAGEVAEFLARHSNVNLTIFTARLCYFWDTDYQEGLCSLSQEGASVKI
MGYKDFVSCWKNFVYSDDEPFKPWKGLQTNFRLLKRRLREILQ
(italic: nucleic acid editing domain)
Human APOBEC-1:
(SEQ ID NO: 96)
MTSEKGPSTGDPTLRRRIEPWEFDVFYDPRELRKEACLLYEIKWGMSRK
IWRSSGKNTTNHVEVNFIKKFTSERDFHPSMSCSITWFLSWSPCWECSQ
AIREFLSRHPGVTLVIYVARLFWHMDQQNRQGLRDLVNSGVTIQIMRAS
EYYHCWRNFVNYPPGDEAHWPQYPPLWMMLYALELHCIILSLPPCLKIS
RRWQNHLTFFRLHLQNCHYQTIPPHILLATGLIHPSVAWR
Mouse APOBEC-1:
(SEQ ID NO: 97)
MSSETGPVAVDPTLRRRIEPHEFEVFFDPRELRKETCLLYEINWGGRHS
VWRHTSQNTSNHVEVNFLEKFTTERYFRPNTRCSITWFLSWSPCGECSR
AITEFLSRHPYVTLFIYIARLYHHTDQRNRQGLRDLISSGVTIQIMTEQ
EYCYCWRNFVNYPPSNEAYWPRYPHLWVKLYVLELYCIILGLPPCLKIL
RRKQPQLTFFTITLQTCHYQRIPPHLLWATGLK
Rat APOBEC-1:
(SEQ ID NO: 98)
MSSETGPVAVDPTLRRRIEPHEFEVFFDPRELRKETCLLYEINWGGRHS
IWRHTSQNTNKHVEVNFIEKFTTERYFCPNTRCSITWFLSWSPCGECSR
AITEFLSRYPHVTLFIYIARLYHHADPRNRQGLRDLISSGVTIQIMTEQ
ESGYCWRNFVNYSPSNEAHWPRYPHLWVRLYVLELYCIILGLPPCLNIL
RRKQPQLTFFTIALQSCHYQRLPPHILWATGLK
Human APOBEC-2:
(SEQ ID NO: 99)
MAQKEEAAVATEAASQNGEDLENLDDPEKLKELIELPPFEIVTGERLPA
NFFKFQFRNVEYSSGRNKTFLCYVVEAQGKGGQVQASRGYLEDEHAAAH
AEEAFFNTILPAFDPALRYNVTWYVSSSPCAACADRIIKTLSKTKNLRL
LILVGRLFMWEEPEIQAALKKLKEAGCKLRIMKPQDFEYVWQNFVEQEE
GESKAFQPWEDIQENFLYYEEKLADILK
Mouse APOBEC-2:
(SEQ ID NO: 100)
MAQKEEAAEAAAPASQNGDDLENLEDPEKLKELIDLPPFEIVTGVRLPV
NFFKFQFRNVEYSSGRNKTFLCYVVEVQSKGGQAQATQGYLEDEHAGAH
AEEAFFNTILPAFDPALKYNVTWYVSSSPCAACADRILKTLSKTKNLRL
LILVSRLFMWEEPEVQAALKKLKEAGCKLRIMKPQDFEYIWQNFVEQEE
GESKAFEPWEDIQENFLYYEEKLADILK
Rat APOBEC-2:
(SEQ ID NO: 101)
MAQKEEAAEAAAPASQNGDDLENLEDPEKLKELIDLPPFEIVTGVRLPV
NFFKFQFRNVEYSSGRNKTFLCYVVEAQSKGGQVQATQGYLEDEHAGAH
AEEAFFNTILPAFDPALKYNVTWYVSSSPCAACADRILKTLSKTKNLRL
LILVSRLFMWEEPEVQAALKKLKEAGCKLRIMKPQDFEYLWQNFVEQEE
GESKAFEPWEDIQENFLYYEEKLADILK
Bovine APOBEC-2:
(SEQ ID NO: 102)
MAQKEEAAAAAEPASQNGEEVENLEDPEKLKELIELPPFEIVTGERLPA
HYFKFQFRNVEYSSGRNKTFLCYVVEAQSKGGQVQASRGYLEDEHATNH
AEEAFFNSIMPTFDPALRYMVTWYVSSSPCAACADRIVKTLNKTKNLRL
LILVGRLFMWEEPEIQAALRKLKEAGCRLRIMKPQDFEYIWQNFVEQEE
GESKAFEPWEDIQENFLYYEEKLADILK
Petromyzon marinus CDA1 (pmCDAl):
(SEQ ID NO: 103)
MTDAEYVRIHEKLDIYTFKKQFFNNKKSVSHRCYVLFELKRRGERRACF
WGYAVNKPQSGTERGIHAEIFSIRKVEEYLRDNPGQFTINWYSSWSPCA
DCAEKILEWYNQELRGNGHTLKIWACKLYYEKNARNQIGLWNLRDNGVG
LNVMVSEHYQCCRKIFIQSSHNQLNENRWLEKTLKRAEKRRSELSFMIQ
VKILHTTKSPAV
Human APOBEC3G D316R D317R:
(SEQ ID NO: 104)
MKPHFRNTVERMYRDTFSYNFYNRPILSRRNTVWLCYEVKTKGPSRPPL
DAKIFRGQVYSELKYHPEMRFFHWFSKWRKLHRDQEYEVTWYISWSPCT
KCTRDMATFLAEDPKVTLTIFVARLYYFWDPDYQEALRSLCQKRDGPRA
TMKFNYDEFQHCWSKFVYSQRELFEPWNNLPKYYILLHFMLGEILRHSM
DPPTFTFNFNNEPWVRGRHETYLCYEVERMHNDTWVLLNQRRGFLCNQA
PHKHGFLEGRHAELCFLDVIPFWKLDLDQDYRVTCFTSWSPCFSCAQEM
AKFISKKHVSLCIFTARIYRRQGRCQEGLRTLAEAGAKISFTYSEFKHC
WDTFVDHQGCPFQPWDGLDEHSQDLSGRLRAILQNQEN
Human APOBEC3G chain A:
(SEQ ID NO: 105)
MDPPTFTFNFNNEPWWGRHETYLCYEVERMHNDTWVLLNQRRGFLCNQA
PHKHGFLEGRHAELCFLDVIPFWKLDLDQDYRVTCFTSWSPCFSCAQEM
AKFISKNKHVSLCIFTARIYDDQGRCQEGLRTLAEAGAKISFTYSEFKH
CWDTFVDHQGCPFQPWDGLDEHSQDLSGRLRAILQ
Human APOBEC3G chain A D120R D121R:
(SEQ ID NO: 106)
MDPPTFTFNFNNEPWVRGRHETYLCYEVERMHNDTWVLLNQRRGFLCNQ
APHKHGFLEGRHAELCFLDVIPFWKLDLDQDYRVTCFTSWSPCFSCAQE
MAKFISKNKHVSLCIFTARIYRRQGRCQEGLRTLAEAGAKISFMTYSEF
KHCWDTFVDHQGCPFQPWDGLDEHSQDLSGRLRAILQ
hAPOBEC-4 (Homo sapiens):
(SEQ ID NO: 107)
MEPIYEEYLANHGTIVKPYYWLSFSLDCSNCPYHIRTGEEARVSLTEFC
QIFGFPYGTTFPQTKHLTFYELKTSSGSLVQKGHASSCTGNYIHPESML
FEMNGYLDSAIYNNDSIRHIILYSNNSPCNEANHCCISKMYNFLITYPG
ITLSIYFSQLYHTEMDFPASAWNREALRSLASLWPRVVLSPISGGIWHS
VLHSFISGVSGSHVFQPILTGRALADRHNAYEINAITGVKPYFTDVLLQ
TKRNPNTKAQEALESYPLNNAFPGQFFQMPSGQLQPNLPPDLRAPVVFV
LVPLRDLPPMHMGQNPNKPRNIVRHLNMPQMSFQETKDLGRLPTGRSVE
IVEITEQFASSKEADEKKKKKGKK
mAPOBEC-4 (Mus musculus):
(SEQ ID NO: 108)
MDSLLMKQKKFLYHFKNVRWAKGRHETYLCYVVKRRDSATSCSLDFGHL
RNKSGCHVELLFLRYISDWDLDPGRCYRVTWFTSWSPCYDCARHVAEFL
RWNPNLSLRIFTARLYFCEDRKAEPEGLRRLHRAGVQIGIMTFKDYFYC
WNTFVENRERTFKAWEGLHENSVRLTRQLRRILLPLYEVDDLRDAFRML
GF
rAPOBEC-4 (Rattus norvegicus):
(SEQ ID NO: 109)
MEPLYEEYLTHSGTIVKPYYWLSVSLNCTNCPYHIRTGEEARVPYTEFH
QTFGFPWSTYPQTKHLTFYELRSSSGNLIQKGLASNCTGSHTHPESMLF
ERDGYLDSLIFHDSNIRHIILYSNNSPCDEANHCCISKMYNFLMNYPEV
TLSVFFSQLYHTENQFPTSAWNREALRGLASLWPQVTLSAISGGIWQSI
LETFVSGISEGLTAVRPFTAGRTLTDRYNAYEINCITEVKPYFTDALHS
WQKENQDQKVWAASENQPLHNTTPAQWQPDMSQDCRTPAVFMLVPYRDL
PPIHVNPSPQKPRTVVRHLNTLQLSASKVKALRKSPSGRPVKKEEARKG
STRSQEANETNKSKWKKQTLFIKSNICHLLEREQKKIGILSSWSV
mfAPOBEC-4 (Macaca fascicularis):
(SEQ ID NO: 110)
MEPTYEEYLANHGTIVKPYYWLSFSLDCSNCPYHIRTGEEARVSLTEFC
QIFGFPYGTTYPQTKHLTFYELKTSSGSLVQKGHASSCTGNYIHPESML
FEMNGYLDSAIYNNDSIRHIILYCNNSPCNEANHCCISKVYNFLITYPG
ITLSIYFSQLYHTEMDFPASAWNREALRSLASLWPRVVLSPISGGIWHS
VLHSFVSGVSGSHVFQPILTGRALTDRYNAYEINAITGVKPFFTDVLLH
TKRNPNTKAQMALESYPLNNAFPGQSFQMTSGIPPDLRAPVVFVLLPLR
DLPPMHMGQDPNKPRNIIRHLNMPQMSFQETKDLERLPTRRSVETVEIT
ERFASSKQAEEKTKKKKGKK
pmCDA-1 (Petromyzon marinus):
(SEQ ID NO: 111)
MAGYECVRVSEKLDFDTFEFQFENLHYATERHRTYVIFDVKPQSAGGRS
RRLWGYIINNPNVCHAELILMSMIDRHLESNPGVYAMTWYMSWSPCANC
SSKLNPWLKNLLEEQGHTLTMHFSRIYDRDREGDHRGLRGLKHVSNSFR
MGVVGRAEVKECLAEYVEASRRTLTWLDTTESMAAKMRRKLFCILVRCA
GMRESGIPLHLFTLQTPLLSGRVVWWRV
pmCDA-2 (Petromyzon marinus):
(SEQ ID NO: 112)
MELREVVDCALASCVRHEPLSRVAFLRCFAAPSQKPRGTVILFYVEGAG
RGVTGGHAVNYNKQGTSIHAEVLLLSAVRAALLRRRRCEDGEEATRGCT
LHCYSTYSPCRDCVEYIQEFGASTGVRVVIHCCRLYELDVNRRRSEAEG
VLRSLSRLGRDFRLMGPRDAIALLLGGRLANTADGESGASGNAWVTETN
VVEPLVDMTGFGDEDLHAQVQRNKQIREAYANYASAVSLMLGELHVDPD
KFPFLAEFLAQTSVEPSGTPRETRGRPRGASSRGPEIGRQRPADFERAL
GAYGLFLHPRIVSREADREEIKRDLIVVMRKHNYQGP
pmCDA-5 (Petromyzon marinus):
(SEQ ID NO: 113)
MAGDENVRVSEKLDFDTFEFQFENLHYATERHRTYVIFDVKPQSAGGRS
RRLWGYIINNPNVCHAELILMSMIDRHLESNPGVYAMTWYMSWSPCANC
SSKLNPWLKNLLEEQGHTLMMHFSRIYDRDREGDHRGLRGLKHVSNSFR
MGVVGRAEVKECLAEYVEASRRTLTWLDTTESMAAKMRRKLFCILVRCA
GMRESGMPLHLFT
yCD (Saccharomyces cerevisiae):
(SEQ ID NO: 114)
MVTGGMASKWDQKGMDIAYEEAALGYKEGGVPIGGCLINNKDGSVLGRG
HNMRFQKGSATLHGEISTLENCGRLEGKVYKDTTLYTTLSPCDMCTGAI
IMYGIPRCVVGENVNFKSKGEKYLQTRGHEVVVVDDERCKKIMKQFIDE
RPQDWFEDIGE
rAPOBEC-1 (delta 177-186):
(SEQ ID NO: 115)
MSSETGPVAVDPTLRRRIEPHEFEVFFDPRELRKETCLLYEINWGGRHS
IWRHTSQNTNKHVEVNFIEKFTTERYFCPNTRCSITWFLSWSPCGECSR
AITEFLSRYPHVTLFIYIARLYHHADPRNRQGLRDLISSGVTIQIMTEQ
ESGYCWRNFVNYSPSNEAHWPRYPHLWVRGLPPCLNILRRKQPQLTFFT
IALQSCHYQRLPPHILWATGLK
rAPOBEC-1 (delta 202-213):
(SEQ ID NO: 116)
MSSETGPVAVDPTLRRRIEPHEFEVFFDPRELRKETCLLYEINWGGRHS
IWRHTSQNTNKHVEVNFIEKFTTERYFCPNTRCSITWFLSWSPCGECSR
AITEFLSRYPHVTLFIYIARLYHHADPRNRQGLRDLISSGVTIQIMTEQ
ESGYCWRNFVNYSPSNEAHWPRYPHLWVRLYVLELYCIILGLPPCLNIL
RRKQPQHYQRLPPHILWATGLK
Mouse APOBEC-3:
(SEQ ID NO: 117)
MGPFCLGCSHRKCYSPIRNLISQETFKFHFKNLGYAKGRKDTFLCYEVT
RKDCDSPVSLHHGVFKNKDNIHAEICFLYWFHDKVLKVLSPREEFKITW
YMSWSPCFECAEQIVRFLATHHNLSLDIFSSRLYNVQDPETQQNLCRLV
QEGAQVAAMDLYEFKKCWKKFVDNGGRRFRPWKRLLTNFRYQDSKLQEI
LRPCYIPVPSSSSSTLSNICLTKGLPETRFCVEGRRMDPLSEEEFYSQF
YNQRVKHLCYYHRMKPYLCYQLEQFNGQAPLKGCLLSEKGKQHAEILFL
DKIRSMELSQVTITCYLTWSPCPNCAWQLAAFKRDRPDLILHIYTSRLY
FHWKRPFQKGLCSLWQSGILVDVMDLPQFTDCWTNFVNPKRPFWPWKGL
EIISRRTQRRLRRIKESWGLQDLVNDFGNLQLGPPMS (italic:
nucleic acid editing domain)

In some embodiments, an adenosine deaminase can comprise all or a portion of an adenosine deaminase ADAR (e.g., ADAR1 or ADAR2). In another embodiment, an adenosine deaminase can comprise all or a portion of an adenosine deaminase ADAT. In some embodiments, an adenosine deaminase can comprise all or a portion of an ADAT from Escherichia coli (EcTadA) comprising one or more of the following mutations: D108N, A106V, D147Y, E155V, L84F, H123Y, I157F, or a corresponding mutation in another adenosine deaminase. The adenosine deaminase can be derived from any suitable organism (e.g., E. coli). In some embodiments, the adenosine deaminase is from Escherichia coli, Staphylococcus aureus, Salmonella typhi, Shewanella putrefaciens, Haemophilus influenzae, Caulobacter crescentus, or Bacillus subtilis. In some embodiments, the adenosine deaminase is from E. coli. In some embodiments, the adenine deaminase is a naturally-occurring adenosine deaminase that includes one or more mutations corresponding to any of the mutations provided herein (e.g., mutations in ecTadA). The corresponding residue in any homologous protein can be identified by e.g., sequence alignment and determination of homologous residues. The mutations in any naturally-occurring adenosine deaminase (e.g., having homology to ecTadA) that corresponds to any of the mutations described herein (e.g., any of the mutations identified in ecTadA) can be generated accordingly. In particular embodiments, the TadA is any one of the TadA described in PCT/US2017/045381 (WO 2018/027078), which is incorporated herein by reference in its entirety. Mutations were identified through rounds of evolution and selection (e.g., TadA*7.10=variant 10 from seventh round of evolution) having desirable adenosine deaminase activity on single stranded DNA as shown in Table 3.

TABLE 3
Genotypes of TadA Variants

| TadA | 23 | 26 | 36 | 37 | 48 | 49 | 51 | 72 | 84 | 87 | 105 | 108 | 123 | 125 | 142 | 145 | 147 | 152 | 155 | 156 | 157 | 16 |
|---|---|---|---|---|---|---|---|---|---|---|---|---|---|---|---|---|---|---|---|---|---|---|
| 0.1 | W | R | H | N | P | | R | N | L | S | A | D | H | G | A | S | D | R | E | I | K | K |
| 0.2 | W | R | H | N | P | | R | N | L | S | A | D | H | G | A | S | D | R | E | I | K | K |
| 1.1 | W | R | H | N | P | | R | N | L | S | A | N | H | G | A | S | D | R | E | I | K | K |
| 1.2 | W | R | H | N | P | | R | N | L | S | V | N | H | G | A | S | D | R | E | I | K | K |
| 2.1 | W | R | H | N | P | | R | N | L | S | V | N | H | G | A | S | Y | R | V | I | K | K |
| 2.2 | W | R | H | N | P | | R | N | L | S | V | N | H | G | A | S | Y | R | V | I | K | K |
| 2.3 | W | R | H | N | P | | R | N | L | S | V | N | H | G | A | S | Y | R | V | I | K | K |
| 2.4 | W | R | H | N | P | | R | N | L | S | V | N | H | G | A | S | Y | R | V | I | K | K |
| 2.5 | W | R | H | N | P | | R | N | L | S | V | N | H | G | A | S | Y | R | V | I | K | K |
| 2.6 | W | R | H | N | P | | R | N | L | S | V | N | H | G | A | S | Y | R | V | I | K | K |
| 2.7 | W | R | H | N | P | | R | N | L | S | V | N | H | G | A | S | Y | R | V | I | K | K |
| 2.8 | W | R | H | N | P | | R | N | L | S | V | N | H | G | A | S | Y | R | V | I | K | K |
| 2.9 | W | R | H | N | P | | R | N | L | S | V | N | H | G | A | S | Y | R | V | I | K | K |
| 2.10 | W | R | H | N | P | | R | N | L | S | V | N | H | G | A | S | Y | R | V | I | K | K |
| 2.11 | W | R | H | N | P | | R | N | L | S | V | N | H | G | A | S | Y | R | V | I | K | K |
| 2.12 | W | R | H | N | P | | R | N | L | S | V | N | H | G | A | S | Y | R | V | I | K | K |
| 3.1 | W | R | H | N | P | | R | N | F | S | V | N | Y | G | A | S | Y | R | V | F | K | K |
| 3.2 | W | R | H | N | P | | R | N | F | S | V | N | Y | G | A | S | Y | R | V | F | K | K |
| 3.3 | W | R | H | N | P | | R | N | F | S | V | N | Y | G | A | S | Y | R | V | F | K | K |
| 3.4 | W | R | H | N | P | | R | N | F | S | V | N | Y | G | A | S | Y | R | V | F | K | K |
| 3.5 | W | R | H | N | P | | R | N | F | S | V | N | Y | G | A | S | Y | R | V | F | K | K |
| 3.6 | W | R | H | N | P | | R | N | F | S | V | N | Y | G | A | S | Y | R | V | F | K | K |
| 3.7 | W | R | H | N | P | | R | N | F | S | V | N | Y | G | A | S | Y | R | V | F | K | K |
| 3.8 | W | R | H | N | P | | R | N | F | S | V | N | Y | G | A | S | Y | R | V | F | K | K |
| 4.1 | W | R | H | N | P | | R | N | L | S | V | N | H | G | N | S | Y | R | V | I | K | K |
| 4.2 | W | G | H | N | P | | R | N | L | S | V | N | H | G | N | S | Y | R | V | I | K | K |
| 4.3 | W | R | H | N | P | | R | N | F | S | V | N | Y | G | N | S | Y | R | V | F | K | K |
| 5.1 | W | R | L | N | P | | L | N | F | S | V | N | Y | G | A | C | Y | R | V | F | N | K |
| 5.2 | W | R | H | S | P | | R | N | F | S | V | N | Y | G | A | S | Y | R | V | F | K | T |
| 5.3 | W | R | L | N | P | | L | N | I | S | V | N | Y | G | A | C | Y | R | V | I | N | K |
| 5.4 | W | R | H | S | P | | R | N | F | S | V | N | Y | G | A | S | Y | R | V | F | K | T |
| 5.5 | W | R | L | N | P | | L | N | F | S | V | N | Y | G | A | C | Y | R | V | F | N | K |
| 5.6 | W | R | L | N | P | | L | N | F | S | V | N | Y | G | A | C | Y | R | V | F | N | K |
| 5.7 | W | R | L | N | P | | L | N | F | S | V | N | Y | G | A | C | Y | R | V | F | N | K |
| 5.8 | W | R | L | N | P | | L | N | F | S | V | N | Y | G | A | C | Y | R | V | F | N | K |
| 5.9 | W | R | L | N | P | | L | N | F | S | V | N | Y | G | A | C | Y | R | V | F | N | K |
| 5.10 | W | R | L | N | P | | L | N | F | S | V | N | Y | G | A | C | Y | R | V | F | N | K |
| 5.11 | W | R | L | N | P | | L | N | F | S | V | N | Y | G | A | C | Y | R | V | F | N | K |
| 5.12 | W | R | L | N | P | | L | N | F | S | V | N | Y | G | A | C | Y | R | V | F | N | K |
| 5.13 | W | R | H | N | P | | L | D | F | S | V | N | Y | A | A | S | Y | R | V | F | K | K |
| 5.14 | W | R | H | N | S | | L | N | F | C | V | N | Y | G | A | S | Y | R | V | F | K | K |
| 6.1 | W | R | H | N | S | | L | N | F | S | V | N | Y | G | N | S | Y | R | V | F | K | K |
| 6.2 | W | R | H | N | T | V | L | N | F | S | V | N | Y | G | N | S | Y | R | V | F | N | K |
| 6.3 | W | R | L | N | S | | L | N | F | S | V | N | Y | G | A | C | Y | R | V | F | N | K |
| 6.4 | W | R | L | N | S | | L | N | F | S | V | N | Y | G | N | C | Y | R | V | F | N | K |
| 6.5 | W | R | L | N | I | V | L | N | F | S | V | N | Y | G | A | C | Y | R | V | F | N | K |
| 6.6 | W | R | L | N | T | V | L | N | F | S | V | N | Y | G | N | C | Y | R | V | F | N | K |
| 7.1 | W | R | L | N | A | | L | N | F | S | V | N | Y | G | A | C | Y | R | V | F | N | K |
| 7.2 | W | R | L | N | A | | L | N | F | S | V | N | Y | G | N | C | Y | R | V | F | N | K |
| 7.3 | I | R | L | N | A | | L | N | F | S | V | N | Y | G | A | C | Y | R | V | F | N | K |
| 7.4 | R | R | L | N | A | | L | N | F | S | V | N | Y | G | A | C | Y | R | V | F | N | K |
| 7.5 | W | R | L | N | A | | L | N | F | S | V | N | Y | G | A | C | Y | H | V | F | N | K |
| 7.6 | W | R | L | N | A | | L | N | I | S | V | N | Y | G | A | C | Y | P | V | I | N | K |
| 7.7 | L | R | L | N | A | | L | N | F | S | V | N | Y | G | A | C | Y | P | V | F | N | K |
| 7.8 | I | R | L | N | A | | L | N | F | S | V | N | Y | G | N | C | Y | R | V | F | N | K |
| 7.9 | L | R | L | N | A | | L | N | F | S | V | N | Y | G | N | C | Y | P | V | F | N | K |
| 7.10 | R | R | L | N | A | | L | N | F | S | V | N | Y | G | A | C | Y | P | V | F | N | K |

In some embodiments, the TadA is provided as a monomer or dimer (e.g., a heterodimer of wild-type E. coli TadA and an engineered TadA variant). In some embodiments, the adenosine deaminase is an eighth generation TadA*8 variant as shown in Table 4 below.

TABLE 4
TadA8* Adenosine Deaminase Variants

| Adenosine | |
|---|---|
| Deaminase | Adenosine Deaminase Description |
| TadA*8.1 | Monomer_TadA*7.10 + Y147T |
| TadA*8.2 | Monomer_TadA*7.10 + Y147R |
| TadA*8.3 | Monomer_TadA*7.10 + Q154S |
| TadA*8.4 | Monomer_TadA*7.10 + Y123H |
| TadA*8.5 | Monomer_TadA*7.10 + V82S |
| TadA*8.6 | Monomer_TadA*7.10 + T166R |
| TadA*8.7 | Monomer_TadA*7.10 + Q154R |
| TadA*8.8 | Monomer_TadA*7.10 + Y147R_Q154R_Y123H |
| TadA*8.9 | Monomer_TadA*7.10 + Y147R_Q154R_I76Y |
| TadA*8.10 | Monomer_TadA*7.10 + Y147R_Q154R_T166R |
| TadA*8.11 | Monomer_TadA*7.10 + Y147T_Q154R |
| TadA*8.12 | Monomer_TadA*7.10 + Y147T_Q154S |
| TadA*8.13 | Monomer_TadA*7.10 + H123H_Y147R_Q154R_I76Y |
| TadA*8.14 | Heterodimer_(WT) + (TadA*7.10 + Y147T) |
| TadA*8.15 | Heterodimer_(WT) + (TadA*7.10 + Y147R) |
| TadA*8.16 | Heterodimer_(WT) + (TadA*7.10 + Q154S) |
| TadA*8.17 | Heterodimer_(WT) + (TadA*7.10 + Y123H) |
| TadA*8.18 | Heterodimer_(WT) + (TadA*7.10 + V82S) |
| TadA*8.19 | Heterodimer_(WT) + (TadA*7.10 + T166R) |
| TadA*8.20 | Heterodimer_(WT) + (TadA*7.10 + Q154R) |
| TadA*8.21 | Heterodimer_(WT) + (TadA*7.10 + |
| | Y147R_Q154R_Y123H) |
| TadA*8.22 | Heterodimer_(WT) + (TadA*7.10 + |
| | Y147R_Q154R_I76Y) |
| TadA*8.23 | Heterodimer_(WT) + (TadA*7.10 + |
| | Y147R_Q154R_T166R) |
| TadA*8.24 | Heterodimer_(WT) + (TadA*7.10 + |
| | Y147T_Q154R) |
| TadA*8.25 | Heterodimer_(WT) + (TadA*7.10 + |
| | Y147T_Q154S) |
| TadA*8.26 | Heterodimer_(WT) + (TadA*7.10 + |
| | H123H_Y147T_Q154R_I76Y) |

In some embodiments, the adenosine deaminase is a ninth generation TadA*9 variant containing an alteration at an amino acid position selected from the following: 21, 23, 25, 38, 51, 54, 70, 71, 72, 72, 94, 124, 133, 138, 139, 146, and 158 of a TadA variant as shown in the reference sequence below:

(SEQ ID NO: 118)

```
      10 20 30 40 50
MSEVEFSHEY WMRHALTLAK RARDEREVPV GAVLVLNNRV
IGEGWNRAIG
      60 70 80 90 100
LHDPTAHAEI MALRQGGLVM QNYRLIDATL YVTFEPCVMC
AGAMIHSRIG
      110 120 130 140 150
RVVFGVRNAK TGAAGSLMDV LHYPGMNHRV EITEGILADE
CAALLCYFFR
      160
MPRQVFNAQK KAQSSTD
```

In one embodiment, the adenosine deaminase variant contains alterations at two or more amino acid positions selected from the following: 21, 23, 25, 38, 51, 54, 70, 71, 72, 94, 124, 133, 138, 139, 146, and 158 of the TadA reference sequence above. In another embodiment, the adenosine deaminase variant contains one or more (e.g., 2, 3, 4) alterations selected from the following: R21N, R23H, E25F, N38G, L51W, P54C, M70V, Q71M, N72K, Y73S, M94V, P124W, T133K, D139L, D139M, C146R, and A158K of SEQ ID NO. 1. In other embodiments, the adenosine deaminase variant further contains one or more of the following alterations: Y147T, Y147R, Q154S, Y123H, and Q154R. In still other embodiments, the adenosine deaminase variant contains a combination of alterations relative to the above TadA reference sequence selected from the following: E25F+V82S+Y123H, T133K+Y147R+Q154R; E25F+V82S+Y123H+Y147R+Q154R; L51W+V82S+Y123H+C146R+Y147R+Q154R; Y73S+V82S+Y123H+Y147R+Q154R; P54C+V82S+Y123H+Y147R+Q154R; N38G+V82T+Y123H+Y147R+Q154R; N72K+V82S+Y123H+D139L+Y147R+Q154R; E25F+V82S+Y123H+D139M+Y147R+Q154R; Q71M+V82S+Y123H+Y147R+Q154R; E25F+V82S+Y123H+T133K+Y147R+Q154R; E25F+V82S+Y123H+Y147R+Q154R; V82S+Y123H+P124W+Y147R+Q154R; L51W+V82S+Y123H+C146R+Y147R+Q154R; P54C+V82S+Y123H+Y147R+Q154R; Y73S+V82S+Y123H+Y147R+Q154R; N38G+V82T+Y123H+Y147R+Q154R; R23H+V82S+Y123H+Y147R+Q154R; R21N+V82S+Y123H+Y147R+Q154R; V82S+Y123H+Y147R+Q154R+A158K; N72K+V82S+Y123H+D139L+Y147R+Q154R; E25F+V82S+Y123H+D139M+Y147R+Q154R; M70V+V82S+M94V+Y123H+Y147R+Q154R; Q71M+V82S+Y123H+Y147R+Q154R; E25F+I76Y+V82S+Y123H+Y147R+Q154R; I76Y+V82T+Y123H+Y147R+Q154R; N38G+I76Y+V82S+Y123H+Y147R+Q154R; R23H+I76Y+V82S+Y123H+Y147R+Q154R; P54C+I76Y+V82S+Y123H+Y147R+Q154R; R21N+I76Y+V82S+Y123H+Y147R+Q154R; I76Y+V82S+Y123H+D138M+Y147R+Q154R; Y72S+I76Y+V82S+Y123H+Y147R+Q154R; E25F+I76Y+V82S+Y123H+Y147R+Q154R; I76Y+V82T+Y123H+Y147R+Q154R; N38G+I76Y+V82S+Y123H+Y147R+Q154R; R23H+I76Y+V82S+Y123H+Y147R+Q154R; P54C+I76Y+V82S+Y123H+Y147R+Q154R; R21N+I76Y+V82S+Y123H+Y147R+Q154R; I76Y+V82S+Y123H+D138M+Y147R+Q154R; Y72S+I76Y+V82S+Y123H+Y147R+Q154R; and V82S+Q154R; N72K_V82S+Y123H+Y147R+Q154R; Q71M_V82S+Y123H+Y147R+Q154R; V82S+Y123H+T133K+Y147R+Q154R; V82S+Y123H+T133K+Y147R+Q154R+A158K; M70V+Q71M+N72K+V82S+Y123H+Y147R+Q154R; N72K_V82S+Y123H+Y147R+Q154R; Q71M_V82S+Y123H+Y147R+Q154R; M70V+V82S+M94V+Y123H+Y147R+Q154R; V82S+Y123H+T133K+Y147R+Q154R; V82S+Y123H+T133K+Y147R+Q154R+A158K; and M70V+Q71M+N72K+V82S+Y123H+Y147R+Q154R. In some embodiments, the deaminase or other polypeptide sequence lacks a methionine, for example when included as a component of a fusion protein. This can alter the numbering of positions. However, the skilled person will understand that such corresponding mutations refer to the same mutation, e.g., Y73S and Y72S and D139M and D138M.

In some embodiments, Cas9 is fused to nuclear localization sequences, including an NLS of the SV40 large T antigen, nucleoplasmin, c-myc, hRNPA1 M9, IBB domain from importin-alpha, NLS of myoma T protein, human p53, c-abl IV, influenza virus NS1, hepatitis virus delta antigen, mouse Mxl, human poly(ADP-ribose) polymerase, steroid hormone receptor (human) glucocorticoid.

In some embodiments, a Cas9 protein is fused to epitope tags including, but not limited to hemagglutinin (HA) tags, histidine (His) tags, FLAG tags, Myc tags, V5 tags, VSV-G tags, SNAP tags, thioredoxin (Trx) tags.

In some embodiments, Cas9 is fused to reporter genes including, but not limited to glutathione-S-transferase (GST), horseradish peroxidase (HRP), chloramphenicol transferase (CAT), HcRed, DsRed, cyan fluorescent protein, yellow fluorescent protein and blue fluorescent protein, green fluorescent protein (GFP), including enhanced versions or superfolded GFP, as well as other modified versions of reporter genes.

In some embodiments, serum half-life of an engineered Cas9 protein is increased by fusion with heterologous proteins such as a human serum albumin protein, transferrin protein, human IgG and/or sialylated petide, such as the carboxy-terminal peptide (CTP, of chorionic gonadotropin p chain).

In some embodiments, serum half-life of an engineered Cas9 protein is decreased by fusion with destabilizing domains, including but not limited to geminin, ubiquitin, FKBP12-L106P, and/or dihydrofolate reductase.

Suitable fusion partners that provide for increased or decreased stability include, but are not limited to degron sequences. Degrons are readily understood by one of ordinary skill in the art to be amino acid sequences that control the stability of the protein of which they are part. For example, the stability of a protein comprising a degron sequence is controlled at least in part by the degron sequence. In some cases, a suitable degron is constitutive such that the degron exerts its influence on protein stability independent of experimental control (i.e., the degron is not drug inducible, temperature inducible, etc.) In some cases, the degron provides the variant Cas9 polypeptide with controllable stability such that the variant Cas9 polypeptide can be turned “on” (i.e., stable) or “off (i.e., unstable, degraded) depending on the desired conditions. For example, if the degron is a temperature sensitive degron, the variant Cas9 polypeptide may be functional (i.e., “on”, stable) below a threshold temperature (e.g., 42° C., 41° C., 40° C., 39° C., 38° C., 37° C., 36° C., 35° C., 34° C., 33° C., 32° C., 31° C., 30° C., etc.) but non-functional (i.e., “off, degraded) above the threshold temperature. As another example, if the degron is a drug inducible degron, the presence or absence of drug can switch the protein from an “off (i.e., unstable) state to an “on” (i.e., stable) state or vice versa. An exemplary drug inducible degron is derived from the FKBP12 protein. The stability of the degron is controlled by the presence or absence of a small molecule that binds to the degron.

Examples of suitable degrons include, but are not limited to those degrons controlled by Shield-1, DHFR, auxins, and/or temperature. Non-limiting examples of suitable degrons are known in the art (e.g., Dohmen et al., Science, 1994. 263(5151): p. 1273-1276: Heat-inducible degron: a method for constructing temperature-sensitive mutants; Schoeber et al., Am J Physiol Renal Physiol. 2009 January; 296(1):F204-11: Conditional fast expression and function of multimeric TRPV5 channels using Shield-1; Chu et al., Bioorg Med Chem Lett. 2008 Nov. 15; 18(22):5941-4: Recent progress with FKBP-derived destabilizing domains; Kanemaki, Pflugers Arch. 2012 Dec. 28: Frontiers of protein expression control with conditional degrons; Yang et al., Mol Cell. 2012 Nov. 30; 48(4):487-8: Titivated for destruction: the methyl degron; Barbour et al., Biosci Rep. 2013 Jan. 18; 33(1): Characterization of the bipartite degron that regulates ubiquitin-independent degradation of thymidylate synthase; and Greussing et al., J Vis Exp. 2012 Nov. 10; (69): Monitoring of ubiquitin-proteasome activity in living cells using a Degron (dgn)-destabilized green fluorescent protein (GFP)-based reporter protein; all of which are hereby incorporated in their entirety by reference).

Exemplary degron sequences have been well-characterized and tested in both cells and animals. Thus, fusing dead Cas9 to a degron sequence produces a “tunable” and “inducible” dead Cas9 polypeptide.

Any of the fusion partners described herein can be used in any desirable combination. As one non-limiting example to illustrate this point, a Cas9 fusion protein can comprise a YFP sequence for detection, a degron sequence for stability, and transcription activator sequence to increase transcription of the target DNA. Furthermore, the number of fusion partners that can be used in a dCas9 fusion protein is unlimited. In some cases, a Cas9 fusion protein comprises one or more (e.g. two or more, three or more, four or more, or five or more) heterologous sequences.

### Target Nucleic Acids

A target nucleic acid is a DNA molecule, RNA molecule, which is single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases either deoxyribonucleotides, ribonucleotides, or analogs thereof. Target nucleic acids may have three-dimensional structure, may include coding or non-coding regions, may include exons, introns, mRNA, tRNA, rRNA, siRNA, shRNA, miRNA, ribozymes, cDNA, plasmids, vectors, exogenous sequences, endogenous sequences. A target nucleic acid can comprise modified nucleotides, include methylated nucleotides, or nucleotide analogs. In some embodiments, a target nucleic acid may be interspersed with non-nucleic acid components.

A target nucleic acid is recognized by CRISPR-Cas9 system and binds Cas9. In some embodiments, it is modified or cleaved or has altered expression due to the binding of Cas9. A target nucleic acid contains a specific recognizable PAM motif, for example, 5′-NGG-3′, 5′-NAGHC-3′, 5′-NRHRRH-3′ or 5′-NNAAA-3′ (H=A, C or T; R=A or G).

### Recombinant Gene Technology

In accordance with the present disclosure, there may be employed conventional molecular biology, microbiology, and recombinant DNA techniques within the skill of the art. Such techniques are described in the literature (see, e.g., Sambrook, Fritsch & Maniatis, Molecular Cloning: A Laboratory Manual, Second Edition (1989) Cold Spring Harbor Laboratory Press, Cold Spring Harbor, N.Y.; DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And 5 Translation (B. D. Hames & S. J. Higgins, eds. (1984)); Animal Cell Culture (R. I. Freshney, ed. (1986)); Immobilized Cells and Enzymes (IRL Press, (1986)); B. Perbal, A Practical Guide To Molecular Cloning (1984); F. M. Ausubel et al. (eds.), Current Protocols in Molecular Biology, John Wiley & Sons, Inc. (1994).

Recombinant expression of a gene, such as a nucleic acid encoding a polypeptide, such as an engineered Cas9 enzyme described herein, can include construction of an expression vector containing a nucleic acid that encodes the polypeptide. Once a polynucleotide has been obtained, a vector for the production of the polypeptide can be produced by recombinant DNA technology using techniques known in the art. Known methods can be used to construct expression vectors containing polypeptide coding sequences and appropriate transcriptional and translational control signals. These methods include, for example, in vitro recombinant DNA techniques, synthetic techniques, and in vivo genetic recombination.

An expression vector can be transferred to a host cell by conventional techniques, and the transfected cells can then be cultured by conventional techniques to produce polypeptides.

In some embodiments, a nucleotide sequence encoding a DNA-targeting RNA and/or Cas9 protein is operably linked to a control element, e.g., a transcriptional control element, such as a promoter. The transcriptional control element may be functional in either a eukaryotic cell, e.g., a mammalian cell; or a prokaryotic cell (e.g., bacterial or archaeal cell). In some embodiments, the eukaryotic cell is a human cell. In some embodiments, a nucleotide sequence encoding a DNA-targeting RNA and/or a novel Cas9 protein is operably linked to multiple control elements that allow expression of the encoded nucleotide sequence in both prokaryotic and eukaryotic cells.

A promoter can be a constitutively active promoter (i.e., a promoter that is constitutively in an active/“ON” state), it may be an inducible promoter (i.e., a promoter whose state, active/“ON” or inactive/“OFF”, is controlled by an external stimulus, e.g., the presence of a particular temperature, compound, or protein.), it may be a spatially restricted promoter (i.e., transcriptional control element, enhancer, etc.)(e.g., tissue specific promoter, cell type specific promoter, etc.), and it may be a temporally restricted promoter (i.e., the promoter is in the “ON” state or “OFF” state during specific stages of embryonic development or during specific stages of a biological process, e.g., hair follicle cycle in mice).

Suitable promoters can be derived from viruses and can therefore be referred to as viral promoters, or they can be derived from any organism, including prokaryotic or eukaryotic organisms. Suitable promoters can be used to drive expression by any RNA polymerase (e.g., pol I, pol II, pol III). Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6) (Miyagishi et al., Nature Biotechnology 20, 497-500 (2002)), an enhanced U6 promoter (e.g., Xia et al., Nucleic Acids Res. 2003 Sep. 1; 31(17)), and/or a human HI promoter (HI).

Examples of inducible promoters include, but are not limited toT7 RNA polymerase promoter, T3 RNA polymerase promoter, Isopropyl-beta-D-thiogalactopyranoside (IPTG)-regulated promoter, lactose induced promoter, heat shock promoter, Tetracycline-regulated promoter (e.g., Tet-ON, Tet-OFF, etc.), Steroid-regulated promoter, Metal-regulated promoter, estrogen receptor-regulated promoter, etc. Inducible promoters can therefore be regulated by molecules including, but not limited to, doxycycline, RNA polymerase, e.g., T7 RNA polymerase, an estrogen receptor and/or an estrogen receptor fusion.

In some embodiments, the promoter is a spatially restricted promoter (i.e., cell type specific promoter, tissue specific promoter, etc.) such that in a multi-cellular organism, the promoter is active (i.e., “ON”) in a subset of specific cells. Spatially restricted promoters may also be referred to as enhancers, transcriptional control elements, control sequences, etc. Any convenient spatially restricted promoter may be used and the choice of suitable promoter (e.g., a brain specific promoter, a promoter that drives expression in a subset of neurons, a promoter that drives expression in the germline, a promoter that drives expression in the lungs, a promoter that drives expression in muscles, a promoter that drives expression in islet cells of the pancreas, etc.) will depend on the organism. Thus, a spatially restricted promoter can be used to regulate the expression of a nucleic acid encoding a subject site-directed polypeptide in a wide variety of different tissues and cell types, depending on the organism. Some spatially restricted promoters are also temporally restricted such that the promoter is in the “ON” state or “OFF” state during specific stages of embryonic development or during specific stages of a biological process (e.g., hair follicle cycle).

For illustration purposes, examples of spatially restricted promoters include, but are not limited to, neuron-specific promoters, adipocyte-specific promoters, cardiomyocyte-specific promoters, smooth muscle-specific promoters, photoreceptor-specific promoters, etc. Neuron-specific spatially restricted promoters include, but are not limited to, a neuron-specific enolase (NSE) promoter, an aromatic amino acid decarboxylase (AADC) promoter, a neurofilament promoter, a synapsin promoter, a thy-1 promoter, a serotonin receptor promoter, a tyrosine hydroxylase promoter (TH), a GnRH promoter, an L7 promoter, a DNMT promoter, an enkephalin promoter, a myelin basic protein (MBP) promoter, a Ca²⁺-calmodulin-dependent protein kinase II-alpha (CamKIIa) promoter and/or a CMV enhancer/platelet-derived growth factor-β promoter.

Adipocyte-specific spatially restricted promoters include, but are not limited to aP2 gene promoter/enhancer, e.g., a region from −5.4 kb to +21 bp of a human aP2 gene, a glucose transporter-4 (GLUT4) promoter, a fatty acid translocase (FAT/CD36) promoter, a stearoyl-CoA desaturase-1 (SCD1) promoter, a leptin promoter, and an adiponectin promoter, an adipsin promoter and/or a resistin promoter.

Cardiomyocyte-specific spatially restricted promoters include, but are not limited to control sequences derived from the following genes: myosin light chain-2, a-myosin heavy chain, AE3, cardiac troponin C, and/or cardiac actin.

Smooth muscle-specific spatially restricted promoters include, but are not limited to an SM22a promoter, a smoothelin promoter, and/or an a-smooth muscle actin promoter.

Photoreceptor-specific spatially restricted promoters include, but are not limited to, a rhodopsin promoter, a rhodopsin kinase promoter, a beta phosphodiesterase gene promoter, a retinitis pigmentosa gene promoter, an interphotoreceptor retinoid-binding protein (IRBP) gene enhancer, and/or an IRBP gene promoter.

### Gene Editing Uses of CRISPR-Cas9

The CRISPR-Cas9 system described herein can be used for gene editing, which can result in a gene silencing event, or an alteration of the expression (e.g., an increase or a decrease) in the expression of a desired target gene. Accordingly, in some embodiments, the CRISPR-Cas9 system described herein is used in a method of altering the expression of a target nucleic acid. In some embodiments the CRISPR-Cas9 system described herein is used in a method of modifying a target nucleic acid in a desired target cell. In some embodiments, the invention provides methods for site-specific modification of a target nucleic acid in eukaryotic cells to effectuate a desired modification in gene expression.

In some embodiments, the invention provides an engineered, non-naturally occurring CRISPR-Cas system comprising: an RNA guide or a nucleic acid encoding the RNA guide, wherein the RNA guide comprises a direct repeat sequence and a spacer sequence capable of hybridizing to a target nucleic acid; and a codon-optimized CRISPR-associated (Cas) protein having at least 80% sequence identity to SEQ ID NO: 1, 2, 3, 4, 5, 6, 7, 8, 9, 10, 11, 12, 13 or 14, and wherein the Cas protein is capable of binding to the RNA guide and of causing a break in the target nucleic acid sequence complementary to the RNA guide.

In some embodiments, the invention provides engineered, non-naturally occurring CRISPR-Cas system comprising: an RNA guide or a nucleic acid encoding the RNA guide, wherein the RNA guide comprises a direct repeat sequence and a spacer sequence capable of hybridizing to a target nucleic acid; and a codon-optimized CRISPR-associated (Cas) protein having at least 80% sequence identity to SEQ ID NO: 1, 2, 3, 4, 5, 6, 7, 8, 9, 10, 11, 12, 13 or 14; wherein the Cas protein is fused to a deaminase, and wherein the Cas protein fusion is capable of binding to the RNA guide and of editing the target nucleic acid sequence complementary to the RNA guide.

In some embodiments, the invention provides a method of altering expression of a target nucleic acid in a eukaryotic cell comprising: contacting the cell with a Cas9 described herein, and an RNA guide or a nucleic acid encoding the RNA guide, wherein the RNA guide comprises a direct repeat sequence and a spacer sequence capable of hybridizing to the target nucleic acid, and wherein the Cas9 protein is capable of binding to the RNA guide and of causing a break in the target nucleic acid sequence complementary to the RNA guide.

In some embodiments, the invention provides a method of altering expression of a target nucleic acid in a eukaryotic cell comprising: contacting the cell with a Cas9 described herein, and an RNA guide or a nucleic acid encoding the RNA guide, wherein the RNA guide comprises a direct repeat sequence and a spacer sequence capable of hybridizing to the target nucleic acid, and wherein the Cas9 protein is capable of binding to the RNA guide and editing the target nucleic acid sequence complementary to the RNA guide.

In some embodiments, the invention provides a method of modifying a target nucleic acid in a eukaryotic cell comprising: contacting the cell with a Cas9 described herein, and an RNA guide or a nucleic acid encoding the RNA guide, wherein the RNA guide comprises a direct repeat sequence and a spacer sequence capable of hybridizing to the target nucleic acid, and wherein the Cas9 protein is capable of binding to the RNA guide and editing the target nucleic acid sequence complementary to the RNA guide.

Accordingly, in some embodiments, the Cas protein has about 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO: 1, 2, 3, 4, 5, 6, 7, 8, 9, 10, 11, 12, 13 or 14. In some embodiments, the Cas protein is identical to SEQ ID NO: 1, 2, 3, 4, 5, 6, 7, 8, 9, 10, 11, 12, 13 or 14.

Suitable guide RNA, Cas9 mutations and fusion proteins for use in the CRISPR-Cas9 system and method are as described throughout this disclosure.

In one aspect, the method comprises binding of the CRISPR-Cas9 to a target nucleic acid and effecting cleavage of a target nucleic acids. In some embodiments, the CRISPR-Cas9 system cleaves target DNA or RNA duplexes by introducing double-stranded breaks. In some embodiments, the CRISPR-Cas9 system cleaves target DNA or RNA by introducing single-stranded breaks or nicks.

In some embodiments, the CRISPR-Cas9 method or system comprises a fusion protein with an effector that modifies target DNA in a site-specific manner, where the modifying activity includes methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, demyristoylation activity, integrase activity, transposase activity, recombinase activity, polymerase activity, ligase activity, helicase activity, or nuclease activity, any of which can modify DNA or a DNA-associated polypeptide (e.g., a histone or DNA binding protein).

In some embodiments, the CRISPR-Cas9 method or system comprises a fusion protein with enzymes that can edit DNA sequences by chemically modifying nucleotide bases, including deaminase enzymes that can modify adenosine or cytosine bases and function as site-specific base editors. For example, APOBECI cytidine deaminase, which usually uses RNA as a substrate, can be targeted to single-stranded and double-stranded DNA when it is fused to Cas9, converting cytidine to uridine directly, and ADAR enzymes deaminate adenosine to inosine. Thus, ‘base editing’ using deaminases enables programmable conversion of one target DNA base into another. Various base editors are known in the art and can be used in the method and systems described herein. Exemplary base editors are described in, for example, Rees and Liu Nature Review Genetics, 2018, 19(12): 770-788, the contents of which are incorporated herein. Accordingly, in some embodiments, the Cas9 enzymes (Seq2Cas9, EhiCas9, SeqCas9, SsiCas9, SinCas9, SsaCas9, Ssc2Cas9, Sor2Cas9, SorCas9, SwaCas9, SscCas9, SgaCas9, LkuCas9 and SsuCas9) described herein is a component of a nucleobase editor. In some embodiments, the base editor is the adenine deaminase TadA8 or TadA9.

In some embodiments, base editing results in the introduction of stop codons to silence genes. In some embodiments, base editing results in altered protein function by altering amino acid sequences.

In some embodiments, the CRISPR-Cas9 method or system comprises epigenetic modification of target DNA by fusion with a histone. In some embodiments, the CRISPR-Cas9 system comprises epigenetic modification of target DNA by fusion with an epigenetic modifying enzyme such as a reader, writer or eraser protein. In some embodiments, the CRISPR-Cas9 system comprises fusion with a histone modifying enzyme to alter the histone modification pattern in a selected region of target DNA. Histone modifications can occur in many different ways including methylation, acetylation, ubiquitination, phosphorylation, and in many different combinations, leading to structural changes in DNA. In some embodiments, histone modification leads to transcriptional repression or activation.

In some embodiments, the CRISPR-Cas9 method or system modulates transcription of target DNA by increasing or decreasing transcription through fusion with transcriptional activator proteins or transcriptional repressor proteins, small molecule/drug-responsive transcriptional regulators, inducible transcription regulators. In some embodiments, the CRISPR-Cas9 system is used to control the expression of a target coding mRNA (i.e. a protein encoding gene) where binding results in increased or decreased gene expression.

In some embodiments, the CRISPR-Cas9 method or system is used to control gene regulation by editing genetic regulatory elements such as promoters or enhancers.

In some embodiments, the CRISPR-Cas9 method or system is used to control the expression of a target non-coding RNA, including tRNA, rRNA, snoRNA, siRNA, miRNA, and long ncRNA.

In some embodiments, the CRISPR-Cas9 method or system is used for targeted engineering of chromatin loop structures. Targeted engineering of chromatin loops between regulatory genomic regions provides a means to manipulate endogenous chromatin structures and enable the formation of new enhancer-promoter connections to overcome genetic deficiencies or inhibit aberrant enhancer-promoter connections.

In some embodiments, CRISPR-Cas9 is used for live cell imaging. Fluorescently labelled Cas9 is targeted to repetitive genomic regions such as centromeres and telomeres to track native chromatin loci throughout the cell cycle and determine differential positioning of transcriptionally active and inactive regions in the 3D nuclear space.

In some embodiments, the CRISPR-Cas9 method or system is used for correction of pathogenic mutations by insertion of beneficial clinical variants or suppressor mutations.

### Nucleobase Editors

Disclosed herein, are novel base editors or nucleobase editors for editing, modifying or altering a target nucleotide sequence of a polynucleotide comprising a Cas9. Described herein is a nucleobase editor or a base editor comprising a polynucleotide programmable nucleotide binding domain (e.g., Cas9) and a nucleobase editing domain (e.g., adenosine deaminase). A polynucleotide programmable nucleotide binding domain (e.g., Cas9), when in conjunction with a bound guide polynucleotide (e.g., gRNA), can specifically bind to a target polynucleotide sequence (i.e., via complementary base pairing between bases of the bound guide nucleic acid and bases of the target polynucleotide sequence) and thereby localize the base editor to the target nucleic acid sequence desired to be edited. In some embodiments, the target polynucleotide sequence comprises single-stranded DNA or double-stranded DNA. In some embodiments, the target polynucleotide sequence comprises RNA. In some embodiments, the target polynucleotide sequence comprises a DNA-RNA hybrid. As most of the known genetic variations associated with human disease are point mutations, methods that can more efficiently and cleanly make precise point mutations are needed. Base editing systems as provided herein provide a new way to provide genome editing without generating double-strand DNA breaks, without requiring a donor DNA template, and without inducing an excess of stochastic insertions and deletions.

The base editors provided herein are capable of modifying a specific nucleotide base without generating a significant proportion of indels. The term “indel(s)”, as used herein, refers to the insertion or deletion of a nucleotide base within a nucleic acid. Such insertions or deletions can lead to frame shift mutations within a coding region of a gene. In some embodiments, it is desirable to generate base editors that efficiently modify (e.g., mutate or deaminate) a specific nucleotide within a nucleic acid, without generating a large number of insertions or deletions (i.e., indels) in the target nucleotide sequence. In certain embodiments, any of the base editors provided herein are capable of generating a greater proportion of intended modifications (e.g., point mutations or deaminations) versus indels.

In some embodiments, any of base editor systems provided herein result in less than 50%, less than 40%, less than 30%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, or less than 0.01% indel formation in the target polynucleotide sequence.

Some aspects of the disclosure are based on the recognition that any of the base editors provided herein are capable of efficiently generating an intended mutation, such as a point mutation, in a nucleic acid (e.g., a nucleic acid within a genome of a subject) without generating a significant number of unintended mutations, such as unintended point mutations. In some embodiments, any of the base editors provided herein are capable of generating at least 0.01% of intended mutations (i.e. at least 0.01% base editing efficiency). In some embodiments, any of the base editors provided herein are capable of generating at least 0.01%, 1%, 2%, 3%, 4%, 5%, 10%, 15%, 20%, 25%, 30%, 40%, 45%, 50%, 60%, 70%, 80%, 90%, 95%, or 99% of intended mutations.

In some embodiments, the base editors provided herein are capable of generating a ratio of intended point mutations to indels that is greater than 1:1. In some embodiments, the base editors provided herein are capable of generating a ratio of intended point mutations to indels that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 8.5:1, at least 9:1, at least 10:1, at least 11:1, at least 12:1, at least 13:1, at least 14:1, at least 15:1, at least 20:1, at least 25:1, at least 30:1, at least 40:1, at least 50:1, at least 100:1, at least 200:1, at least 300:1, at least 400:1, at least 500:1, at least 600:1, at least 700:1, at least 800:1, at least 900:1, or at least 1000:1, or more.

The number of intended mutations and indels can be determined using any suitable method, for example, as described in International PCT Application Nos. PCT/2017/045381 (WO2018/027078) and PCT/US2016/058344 (WO2017/070632); Komor, A. C., et al., “Programmable editing of a target base in genomic DNA without double-stranded DNA cleavage” Nature 533, 420-424 (2016); Gaudelli, N. M., et al., “Programmable base editing of A⋅T to G⋅C in genomic DNA without DNA cleavage” Nature 551, 464-471 (2017); and Komor, A. C., et al., “Improved base excision repair inhibition and bacteriophage Mu Gam protein yields C:G-to-T:A base editors with higher efficiency and product purity” Science Advances 3:eaao4774 (2017); the entire contents of which are hereby incorporated by reference.

In some embodiments, to calculate indel frequencies, sequencing reads are scanned for exact matches to two 10-bp sequences that flank both sides of a window in which indels can occur. If no exact matches are located, the read is excluded from analysis. If the length of this indel window exactly matches the reference sequence the read is classified as not containing an indel. If the indel window is two or more bases longer or shorter than the reference sequence, then the sequencing read is classified as an insertion or deletion, respectively. In some embodiments, the base editors provided herein can limit formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor.

The number of indels formed at a target nucleotide region can depend on the amount of time a nucleic acid (e.g., a nucleic acid within the genome of a cell) is exposed to a base editor. In some embodiments, the number or proportion of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing the target nucleotide sequence (e.g., a nucleic acid within the genome of a cell) to a base editor. It should be appreciated that the characteristics of the base editors as described herein can be applied to any of the fusion proteins, or methods of using the fusion proteins provided herein.

### Therapeutic Applications

The CRISPR-Cas9 methods or systems described herein can have various therapeutic applications. Accordingly, in some embodiments, a method of treating a disorder or a disease in a subject in need thereof is provided, the method comprising administering to the subject a CRISPR-Cas9 system comprising a Cas9 as described herein, wherein the guide RNA is complementary to at least 10 nucleotides of a target nucleic acid associated with the condition or disease; wherein the Cas protein associates with the guide RNA; wherein the guide RNA binds to the target nucleic acid; wherein the Cas protein causes a break in the target nucleic acid, optionally wherein the Cas9 is an inactive Cas9 (dCas9) fused to a deaminase and results in one or more base edits in the target nucleic acid, thereby treating the disorder or disease.

In some embodiments, the CRISPR-Cas9 methods or systems can be used to treat various diseases and disorders, e.g., genetic disorders (e.g., monogenetic diseases), diseases that can be treated by nuclease activity, and various cancers, etc.

In some embodiments, the CRISPR methods or systems described herein can be used to edit a target nucleic acid to modify the target nucleic acid (e.g., by inserting, deleting, or mutating one or more nucleic acid residues). For example, in some embodiments the CRISPR systems described herein comprise an exogenous donor template nucleic acid (e.g., a DNA molecule or a RNA molecule), which comprises a desirable nucleic acid sequence. Upon resolution of a cleavage event induced with the CRISPR system described herein, the molecular machinery of the cell will utilize the exogenous donor template nucleic acid in repairing and/or resolving the cleavage event. Alternatively, the molecular machinery of the cell can utilize an endogenous template in repairing and/or resolving the cleavage event. In some embodiments, the CRISPR systems described herein may be used to alter a target nucleic acid resulting in an insertion, a deletion, and/or a point mutation). In some embodiments, the insertion is a scarless insertion (i.e., the insertion of an intended nucleic acid sequence into a target nucleic acid resulting in no additional unintended nucleic acid sequence upon resolution of the cleavage event). Donor template nucleic acids may be double stranded or single stranded nucleic acid molecules (e.g., DNA or RNA). In some embodiments, the CRISPR methods or systems described herein comprise a nucleobase editor. For example, in some embodiments, the Cas9 proteins described herein are fused to a polypeptide having nucleobase editing activity.

In one aspect, the CRISPR methods or systems described herein can be used for treating a disease caused by overexpression of RNAs, toxic RNAs, and/or mutated RNAs (e.g., splicing defects or truncations).

In some embodiments, the CRISPR methods or systems described herein can also target trans-acting mutations affecting RNA-dependent functions that cause various diseases.

In some embodiments, the CRISPR methods or systems described herein can also be used to target mutations disrupting the cis-acting splicing codes that can cause splicing defects and diseases.

The CRISPR methods or systems described herein can further be used for antiviral activity, in particular against RNA viruses. The CRISPR-associated proteins can target the viral RNAs using suitable RNA guides selected to target viral RNA sequences.

The CRISPR methods or systems described herein can also be used to treat a cancer in a subject (e.g., a human subject). For example, the CRISPR-associated proteins described herein can be programmed with crRNA targeting a RNA molecule that is aberrant (e.g., comprises a point mutation or are alternatively-spliced) and found in cancer cells to induce cell death in the cancer cells (e.g., via apoptosis).

Further, the CRISPR methods or systems described herein can also be used to treat an infectious disease in a subject. For example, the CRISPR-associated proteins described herein can be programmed with crRNA targeting a RNA molecule expressed by an infectious agent (e.g., a bacteria, a virus, a parasite or a protozoan) in order to target and induce cell death in the infectious agent cell. The CRISPR systems may also be used to treat diseases where an intracellular infectious agent infects the cells of a host subject. By programming the CRISPR-associated protein to target a RNA molecule encoded by an infectious agent gene, cells infected with the infectious agent can be targeted and cell death induced.

Furthermore, in vitro RNA sensing assays can be used to detect specific RNA substrates. The CRISPR-associated proteins can be used for RNA-based sensing in living cells. Examples of applications are diagnostics by sensing of, for examples, disease-specific RNAs.

In applications in which it is desirable to insert a polynucleotide sequence into a target DNA sequence, a polynucleotide comprising a donor sequence to be inserted is also provided to the cell. By a “donor sequence” or “donor polynucleotide” it is meant a nucleic acid sequence to be inserted at the cleavage site induced by a site-directed modifying polypeptide. The donor polynucleotide will contain sufficient homology to a genomic sequence at the cleavage site, e.g. 70%, 80%, 85%, 90%, 95%, or 100% homology with the nucleotide sequences flanking the cleavage site, e.g. within about 50 bases or less of the cleavage site, e.g. within about 30 bases, within about 15 bases, within about 10 bases, within about 5 bases, or immediately flanking the cleavage site, to support homology-directed repair between it and the genomic sequence to which it bears homology. Approximately 25, 50, 100, or 200 nucleotides, or more than 200 nucleotides, of sequence homology between a donor and a genomic sequence (or any integral value between 10 and 200 nucleotides, or more) will support homology-directed repair. Donor sequences can be of any length, e.g. 10 nucleotides or more, 50 nucleotides or more, 100 nucleotides or more, 250 nucleotides or more, 500 nucleotides or more, 1000 nucleotides or more, 5000 nucleotides or more, etc.

The donor sequence is typically not identical to the genomic sequence that it replaces. Rather, the donor sequence may contain at least one or more single base changes, insertions, deletions, inversions or rearrangements with respect to the genomic sequence, so long as sufficient homology is present to support homology-directed repair. In some embodiments, the donor sequence comprises a non-homologous sequence flanked by two regions of homology, such that homology-directed repair between the target DNA region and the two flanking sequences results in insertion of the non-homologous sequence at the target region. Donor sequences may also comprise a vector backbone containing sequences that are not homologous to the DNA region of interest and that are not intended for insertion into the DNA region of interest. Generally, the homologous region(s) of a donor sequence will have at least 50% sequence identity to a genomic sequence with which recombination is desired. In certain embodiments, 60%, 70%, 80%, 90%, 95%, 98%, 99%, or 99.9% sequence identity is present. Any value between 1% and 100% sequence identity can be present, depending upon the length of the donor polynucleotide.

The donor sequence may comprise certain sequence differences as compared to the genomic sequence, e.g. restriction sites, nucleotide polymorphisms, selectable markers (e.g., drug resistance genes, fluorescent proteins, enzymes etc.), etc., which may be used to assess for successful insertion of the donor sequence at the cleavage site or in some cases may be used for other purposes (e.g., to signify expression at the targeted genomic locus). In some cases, if located in a coding region, such nucleotide sequence differences will not change the amino acid sequence, or will make silent amino acid changes (i.e., changes which do not affect the structure or function of the protein). Alternatively, these sequences differences may include flanking recombination sequences such as FLPs, loxP sequences, or the like, that can be activated at a later time for removal of the marker sequence.

The donor sequence may be provided to the cell as single-stranded DNA, single-stranded RNA, double-stranded DNA, or double-stranded RNA. It may be introduced into a cell in linear or circular form. If introduced in linear form, the ends of the donor sequence may be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. Additional methods for protecting exogenous polynucleotides from degradation include, but are not limited to, addition of terminal amino group(s) and the use of modified internucleotide linkages such as, for example, phosphorothioates, phosphor amidates, and O-methyl ribose or deoxyribose residues. As an alternative to protecting the termini of a linear donor sequence, additional lengths of sequence may be included outside of the regions of homology that can be degraded without impacting recombination. A donor sequence can be introduced into a cell as part of a vector molecule having additional sequences such as, for example, replication origins, promoters and genes encoding antibiotic resistance. Moreover, donor sequences can be introduced as naked nucleic acid, as nucleic acid complexed with an agent such as a liposome or poloxamer, or can be delivered by viruses (e.g., adenovirus, AAV), as described above for nucleic acids encoding a DNA-targeting RNA and/or site-directed modifying polypeptide and/or donor polynucleotide.

Following the methods described above, a DNA region of interest may be cleaved and modified, i.e. “genetically modified”, ex vivo. In some embodiments, as when a selectable marker has been inserted into the DNA region of interest, the population of cells may be enriched for those comprising the genetic modification by separating the genetically modified cells from the remaining population. Prior to enriching, the “genetically modified” cells may make up only about 1% or more (e.g., 2% or more, 3% or more, 4% or more, 5% or more, 6% or more, 7% or more, 8% or more, 9% or more, 10% or more, 15% or more, or 20% or more) of the cellular population. Separation of “genetically modified” cells may be achieved by any convenient separation technique appropriate for the selectable marker used. For example, if a fluorescent marker has been inserted, cells may be separated by fluorescence activated cell sorting, whereas if a cell surface marker has been inserted, cells may be separated from the heterogeneous population by affinity separation techniques, e.g. magnetic separation, affinity chromatography, “panning” with an affinity reagent attached to a solid matrix, or other convenient technique. Techniques providing accurate separation include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g. propidium iodide). Any technique may be employed which is not unduly detrimental to the viability of the genetically modified cells. Cell compositions that are highly enriched for cells comprising modified DNA are achieved in this manner. By “highly enriched”, it is meant that the genetically modified cells will be 70% or more, 75% or more, 80% or more, 85% or more, 90% or more of the cell composition, for example, about 95% or more, or 98% or more of the cell composition. In other words, the composition may be a substantially pure composition of genetically modified cells.

Genetically modified cells produced by the methods described herein may be used immediately. Alternatively, the cells may be frozen at liquid nitrogen temperatures and stored for long periods of time, being thawed and capable of being reused. In such cases, the cells will usually be frozen in 10% dimethylsulfoxide (DMSO), 50% serum, 40% buffered medium, or some other such solution as is commonly used in the art to preserve cells at such freezing temperatures, and thawed in a manner as commonly known in the art for thawing frozen cultured cells.

The genetically modified cells may be cultured in vitro under various culture conditions. The cells may be expanded in culture, i.e. grown under conditions that promote their proliferation. Culture medium may be liquid or semi-solid, e.g. containing agar, methylcellulose, etc. The cell population may be suspended in an appropriate nutrient medium, such as Iscove's modified DMEM or RPMI 1640, normally supplemented with fetal calf serum (about 5-10%),

L-glutamine, a thiol, particularly 2-mercaptoethanol, and antibiotics, e.g. penicillin and streptomycin. The culture may contain growth factors to which the regulatory T cells are responsive. Growth factors, as defined herein, are molecules capable of promoting survival, growth and/or differentiation of cells, either in culture or in the intact tissue, through specific effects on a transmembrane receptor. Growth factors include polypeptides and non-polypeptide factors.

Cells that have been genetically modified in this way may be transplanted to a subject for purposes such as gene therapy, e.g. to treat a disease or as an antiviral, antipathogenic, or anticancer therapeutic, for the production of genetically modified organisms in agriculture, or for biological research. The subject may be a neonate, a juvenile, or an adult. Of particular interest are mammalian subjects. Mammalian species that may be treated with the present methods include canines and felines; equines; bovines; ovines; etc. and primates, particularly humans. Animal models, particularly small mammals (e.g. mouse, rat, guinea pig, hamster, lagomorpha (e.g., rabbit), etc.) may be used for experimental investigations.

Cells may be provided to the subject alone or with a suitable substrate or matrix, e.g. to support their growth and/or organization in the tissue to which they are being transplanted. Usually, at least 1×10³ cells will be administered, for example 5×10³ cells, 1×10⁴ cells, 5×10⁴ cells, 1×10⁵ cells, 1×10⁶ cells or more. The cells may be introduced to the subject via any of the following routes: parenteral, subcutaneous, intravenous, intracranial, intraspinal, intraocular, or into spinal fluid. The cells may be introduced by injection, catheter, or the like. Cells may also be introduced into an embryo (e.g., a blastocyst) for the purpose of generating a transgenic animal (e.g., a transgenic mouse).

The number of administrations of treatment to a subject may vary. Introducing the genetically modified cells into the subject may be a one-time event; but in certain situations, such treatment may elicit improvement for a limited period of time and require an on-going series of repeated treatments. In other situations, multiple administrations of the genetically modified cells may be required before an effect is observed. The exact protocols depend upon the disease or condition, the stage of the disease and parameters of the individual subject being treated.

In other aspects of the invention, the DNA-targeting RNA and/or site-directed modifying polypeptide and/or donor polynucleotide are employed to modify cellular DNA in vivo, again for purposes such as gene therapy, e.g. to treat a disease or as an antiviral, antipathogenic, or anticancer therapeutic, for the production of genetically modified organisms in agriculture, or for biological research. In these in vivo embodiments, a DNA-targeting RNA and/or site-directed modifying polypeptide and/or donor polynucleotide are administered directly to the individual. A DNA-targeting RNA and/or site-directed modifying polypeptide and/or donor polynucleotide may be administered by any of a number of well-known methods in the art for the administration of peptides, small molecules and nucleic acids to a subject. A DNA-targeting RNA and/or site-directed modifying polypeptide and/or donor polynucleotide can be incorporated into a variety of formulations. More particularly, a DNA-targeting RNA and/or site-directed modifying polypeptide and/or donor polynucleotide of the present invention can be formulated into pharmaceutical compositions by combination with appropriate pharmaceutically acceptable carriers or diluents.

Pharmaceutical preparations are compositions that include one or more a DNA-targeting RNA and/or site-directed modifying polypeptide and/or donor polynucleotide present in a pharmaceutically acceptable vehicle. “Pharmaceutically acceptable vehicles” may be vehicles approved by a regulatory agency of the Federal or a state government or listed in the U.S.

Pharmacopeia or other generally recognized pharmacopeia for use in mammals, such as humans. The term “vehicle” refers to a diluent, adjuvant, excipient, or carrier with which a compound of the invention is formulated for administration to a mammal. Such pharmaceutical vehicles can be lipids, e.g. liposomes, e.g. liposome dendrimers; liquids, such as water and oils, including those of petroleum, animal, vegetable or synthetic origin, such as peanut oil, soybean oil, mineral oil, sesame oil and the like, saline; gum acacia, gelatin, starch paste, talc, keratin, colloidal silica, urea, and the like. In addition, auxiliary, stabilizing, thickening, lubricating and coloring agents may be used. Pharmaceutical compositions may be formulated into preparations in solid, semisolid, liquid or gaseous forms, such as tablets, capsules, powders, granules, ointments, solutions, suppositories, injections, inhalants, gels, microspheres, and aerosols. As such, administration of the a DNA-targeting RNA and/or site-directed modifying polypeptide and/or donor polynucleotide can be achieved in various ways, including oral, buccal, rectal, parenteral, intraperitoneal, intradermal, transdermal, intratracheal, intraocular, etc., administration. The active agent may be systemic after administration or may be localized by the use of regional administration, intramural administration, or use of an implant that acts to retain the active dose at the site of implantation. The active agent may be formulated for immediate activity or it may be formulated for sustained release.

For some conditions, particularly central nervous system conditions, it may be necessary to formulate agents to cross the blood-brain barrier (BBB). One strategy for drug delivery through the blood-brain barrier (BBB) entails disruption of the BBB, either by osmotic means such as mannitol or leukotrienes, or biochemically by the use of vasoactive substances such as bradykinin. The potential for using BBB opening to target specific agents to brain tumors is also an option. A BBB disrupting agent can be co-administered with the therapeutic compositions of the invention when the compositions are administered by intravascular injection. Other strategies to go through the BBB may entail the use of endogenous transport systems, including Caveolin-1 mediated transcytosis, carrier-mediated transporters such as glucose and amino acid carriers, receptor-mediated transcytosis for insulin or transferrin, and active efflux transporters such as p-glycoprotein. Active transport moieties may also be conjugated to the therapeutic compounds for use in the invention to facilitate transport across the endothelial wall of the blood vessel.

Alternatively, drug delivery of therapeutics agents behind the BBB may be by local delivery, for example by intrathecal delivery.

Typically, an effective amount of a DNA-targeting RNA and/or site-directed modifying polypeptide and/or donor polynucleotide are provided. As discussed above with regard to ex vivo methods, an effective amount or effective dose of a DNA-targeting RNA and/or site-directed modifying polypeptide and/or donor polynucleotide in vivo is the amount to induce a 2 fold increase or more in the amount of recombination observed between two homologous sequences relative to a negative control, e.g. a cell contacted with an empty vector or irrelevant polypeptide. The amount of recombination may be measured by any convenient method, e.g. as described above and known in the art. The calculation of the effective amount or effective dose of a DNA-targeting RNA and/or site-directed modifying polypeptide and/or donor polynucleotide to be administered is within the skill of one of ordinary skill in the art, and will be routine to those persons skilled in the art. The final amount to be administered will be dependent upon the route of administration and upon the nature of the disorder or condition that is to be treated.

The effective amount given to a particular patient will depend on a variety of factors, several of which will differ from patient to patient. A competent clinician will be able to determine an effective amount of a therapeutic agent to administer to a patient to halt or reverse the progression the disease condition as required. Utilizing LD50 animal data, and other information available for the agent, a clinician can determine the maximum safe dose for an individual, depending on the route of administration. For instance, an intravenously administered dose may be more than an intrathecally administered dose, given the greater body of fluid into which the therapeutic composition is being administered. Similarly, compositions which are rapidly cleared from the body may be administered at higher doses, or in repeated doses, in order to maintain a therapeutic concentration. Utilizing ordinary skill, the competent clinician will be able to optimize the dosage of a particular therapeutic in the course of routine clinical trials.

For inclusion in a medicament, a DNA-targeting RNA and/or site-directed modifying polypeptide and/or donor polynucleotide may be obtained from a suitable commercial source. As a general proposition, the total pharmaceutically effective amount of the a DNA-targeting RNA and/or site-directed modifying polypeptide and/or donor polynucleotide administered parenterally per dose will be in a range that can be measured by a dose response curve.

Therapies based on a DNA-targeting RNA and/or site-directed modifying polypeptide and/or donor polynucleotides, i.e. preparations of a DNA-targeting RNA and/or site-directed modifying polypeptide and/or donor polynucleotide to be used for therapeutic administration, must be sterile. Sterility is readily accomplished by filtration through sterile filtration membranes (e.g., 0.2 m membranes). Therapeutic compositions generally are placed into a container having a sterile access port, for example, an intravenous solution bag or vial having a stopper pierceable by a hypodermic injection needle. The therapies based on a DNA-targeting RNA and/or site-directed modifying polypeptide and/or donor polynucleotide may be stored in unit or multi-dose containers, for example, sealed ampules or vials, as an aqueous solution or as a lyophilized formulation for reconstitution. As an example of a lyophilized formulation, 10-mL vials are filled with 5 ml of sterile-filtered 1% (w/v) aqueous solution of compound, and the resulting mixture is lyophilized. The infusion solution is prepared by reconstituting the lyophilized compound using bacteriostatic Water-for-Injection.

Pharmaceutical compositions can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, and enhance solubility or uptake). Examples of such modifications or complexing agents include sulfate, gluconate, citrate and phosphate. The nucleic acids or polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Therapies that exhibit large therapeutic indices are preferred.

The data obtained from cell culture and/or animal studies can be used in formulating a range of dosages for humans. The dosage of the active ingredient typically lines within a range of circulating concentrations that include the ED50 with low toxicity. The dosage can vary within this range depending upon the dosage form employed and the route of administration utilized.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

### Delivery Systems

The CRISPR systems described herein, or components thereof, nucleic acid molecules thereof, and/or nucleic acid molecules encoding or providing components thereof, CRISPR-associated proteins, or RNA guides, can be delivered by various delivery systems such as vectors, e.g., plasmids and delivery vectors. Exemplary embodiments are described below. The CRISPR systems (e.g., including the Cas9 comprising nucleobase editor described herein) can be encoded on a nucleic acid that is contained in a viral vector. Viral vectors can include lentivirus, Adenovirus, Retrovirus, and Adeno-associated viruses (AAVs). Viral vectors can be selected based on the application. For example, AAVs are commonly used for gene delivery in vivo due to their mild immunogenicity. Adenoviruses are commonly used as vaccines because of the strong immunogenic response they induce. Packaging capacity of the viral vectors can limit the size of the base editor that can be packaged into the vector. For example, the packaging capacity of the AAVs is ˜4.5 kb including two 145 base inverted terminal repeats (JTRs).

AAV is a small, single-stranded DNA dependent virus belonging to the parvovirus family. The 4.7 kb wild-type (wt) AAV genome is made up of two genes that encode four replication proteins and three capsid proteins, respectively, and is flanked on either side by 145-bp inverted terminal repeats (JTRs). The virion is composed of three capsid proteins, Vp1, Vp2, and Vp3, produced in a 1:1:10 ratio from the same open reading frame but from differential splicing (Vp1) and alternative translational start sites (Vp2 and Vp3, respectively). Vp3 is the most abundant subunit in the virion and participates in receptor recognition at the cell surface defining the tropism of the virus. A phospholipase domain, which functions in viral infectivity, has been identified in the unique N terminus of Vp1.

Similar to wt AAV, recombinant AAV (rAAV) utilizes the cis-acting 145-bp JTRs to flank vector transgene cassettes, providing up to 4.5 kb for packaging of foreign DNA. Subsequent to infection, rAAV can express a fusion protein of the invention and persist without integration into the host genome by existing episomally in circular head-to-tail concatemers. Although there are numerous examples of rAAV success using this system, in vitro and in vivo, the limited packaging capacity has limited the use of AAV-mediated gene delivery when the length of the coding sequence of the gene is equal or greater in size than the wt AAV genome.

The small packaging capacity of AAV vectors makes the delivery of a number of genes that exceed this size and/or the use of large physiological regulatory elements challenging. These challenges can be addressed, for example, by dividing the protein(s) to be delivered into two or more fragments, wherein the N-terminal fragment is fused to a split intein-N and the C-terminal fragment is fused to a split intein-C. These fragments are then packaged into two or more AAV vectors. As used herein, “intein” refers to a self-splicing protein intron (e.g., peptide) that ligates flanking N-terminal and C-terminal exteins (e.g., fragments to be joined). The use of certain inteins for joining heterologous protein fragments is described, for example, in Wood et al., J. Biol. Chem. 289(21); 14512-9 (2014). For example, when fused to separate protein fragments, the inteins IntN and IntC recognize each other, splice themselves out and simultaneously ligate the flanking N- and C-terminal exteins of the protein fragments to which they were fused, thereby reconstituting a full-length protein from the two protein fragments. Other suitable inteins will be apparent to a person of skill in the art.

In some embodiments, the CRISPR system of the invention can vary in length. In some embodiments, a protein fragment ranges from 2 amino acids to about 1000 amino acids in length. In some embodiments, a protein fragment ranges from about 5 amino acids to about 500 amino acids in length. In some embodiments, a protein fragment ranges from about 20 amino acids to about 200 amino acids in length. In some embodiments, a protein fragment ranges from about 10 amino acids to about 100 amino acids in length. Suitable protein fragments of other lengths will be apparent to a person of skill in the art.

In some embodiments, a portion or fragment of a nuclease (e.g., Cas9) is fused to an intein. The nuclease can be fused to the N-terminus or the C-terminus of the intein. In some embodiments, a portion or fragment of a fusion protein is fused to an intein and fused to an AAV capsid protein. The intein, nuclease and capsid protein can be fused together in any arrangement (e.g., nuclease-intein-capsid, intein-nuclease-capsid, capsid-intein-nuclease, etc.). In some embodiments, the N-terminus of an intein is fused to the C-terminus of a fusion protein and the C-terminus of the intein is fused to the N-terminus of an AAV capsid protein.

In one embodiment, dual AAV vectors are generated by splitting a large transgene expression cassette in two separate halves (5′ and 3′ ends, or head and tail), where each half of the cassette is packaged in a single AAV vector (of <5 kb). The re-assembly of the full-length transgene expression cassette is then achieved upon co-infection of the same cell by both dual AAV vectors followed by: (1) homologous recombination (HR) between 5′ and 3′ genomes (dual AAV overlapping vectors); (2) ITR-mediated tail-to-head concatemerization of 5′ and 3′ genomes (dual AAV trans-splicing vectors); or (3) a combination of these two mechanisms (dual AAV hybrid vectors). The use of dual AAV vectors in vivo results in the expression of full-length proteins. The use of the dual AAV vector platform represents an efficient and viable gene transfer strategy for transgenes of >4.7 kb in size.

The disclosed strategies for designing CRISPR systems including the Cas9 described herein can be useful for generating CRISPR systems capable of being packaged into a viral vector. The use of RNA or DNA viral based systems for the delivery of a base editor takes advantage of highly evolved processes for targeting a virus to specific cells in culture or in the host and trafficking the viral payload to the nucleus or host cell genome. Viral vectors can be administered directly to cells in culture, patients (in vivo), or they can be used to treat cells in vitro, and the modified cells can optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (See, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700).

Retroviral vectors, especially lentiviral vectors, can require polynucleotide sequences smaller than a given length for efficient integration into a target cell. For example, retroviral vectors of length greater than 9 kb can result in low viral titers compared with those of smaller size. In some aspects, a CRISPR system (e.g., including the Cas9 disclosed herein) of the present disclosure is of sufficient size so as to enable efficient packaging and delivery into a target cell via a retroviral vector. In some cases, a Cas9 is of a size so as to allow efficient packing and delivery even when expressed together with a guide nucleic acid and/or other components of a targetable nuclease system.

In applications where transient expression is preferred, adenoviral based systems can be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors can also be used to transduce cells with target nucleic acids, e.g., in the in vitro production of nucleic acids and peptides, and for in vivo and ex vivo gene therapy procedures (See, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). The construction of recombinant AAV vectors is described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

A CRISPR system (e.g., including the Cas9 disclosed herein) described herein can therefore be delivered with viral vectors. One or more components of the base editor system can be encoded on one or more viral vectors. For example, a base editor and guide nucleic acid can be encoded on a single viral vector. In other cases, the base editor and guide nucleic acid are encoded on different viral vectors. In either case, the base editor and guide nucleic acid can each be operably linked to a promoter and terminator.

The combination of components encoded on a viral vector can be determined by the cargo size constraints of the chosen viral vector.

### Non-Viral Delivery of Base Editors

Non-viral delivery approaches for CRISPR are also available. One important category of non-viral nucleic acid vectors are nanoparticles, which can be organic or inorganic. Nanoparticles are well known in the art. Any suitable nanoparticle design can be used to deliver genome editing system components or nucleic acids encoding such components. For instance, organic (e.g. lipid and/or polymer) nanoparticles can be suitable for use as delivery vehicles in certain embodiments of this disclosure. Exemplary lipids for use in nanoparticle formulations, and/or gene transfer are shown in Table 5 (below).

TABLE 5
Lipids Used for Gene Transfer

| Lipid | Abbreviation | Feature |
|---|---|---|
| 1,2-Dioleoyl-sn-glycero-3-phosphatidylcholine | DOPC | Helper |
| 1,2-Dioleoyl-sn-glycero-3-phosphatidylethanolamine | DOPE | Helper |
| Cholesterol | | Helper |
| N-[1-(2,3-Dioleyloxy)prophyl]N,N,N-trimethylammonium | DOTMA | Cationic |
| chloride | | |
| 1,2-Dioleoyloxy-3-trimethylammonium-propane | DOTAP | Cationic |
| Dioctadecylamidoglycylspermine | DOGS | Cationic |
| N-(3-Aminopropyl)-N,N-dimethyl-2,3-bis(dodecyloxy)-1- | GAP-DLRIE | Cationic |
| propanaminium bromide | | |
| Cetyltrimethylammonium bromide | CTAB | Cationic |
| 6-Lauroxyhexyl ornithinate | LHON | Cationic |
| 1-(2,3-Dioleoyloxypropyl)-2,4,6-trimethylpyridinium | 2Oc | Cationic |
| 2,3-Dioleyloxy-N-[2(sperminecarboxamido-ethyl]-N,N- | DOSPA | Cationic |
| dimethyl-1-propanaminium trifluoroacetate | | |
| 1,2-Dioleyl-3-trimethylammonium-propane | DOPA | Cationic |
| N-(2-Hydroxyethyl)-N,N-dimethyl-2,3-bis(tetradecyloxy)-1- | MDRIE | Cationic |
| propanaminium bromide | | |
| Dimyristooxypropyl dimethyl hydroxyethyl ammonium bromide | DMRI | Cationic |
| 3β-[N-(N′,N′-Dimethylaminoethane)-carbamoyl]cholesterol | DC-Chol | Cationic |
| Bis-guanidium-tren-cholesterol | BGTC | Cationic |
| 1,3-Diodeoxy-2-(6-carboxy-spermyl)-propylamide | DOSPER | Cationic |
| Dimethyloctadecylammonium bromide | DDAB | Cationic |
| Dioctadecylamidoglicylspermidin | DSL | Cationic |
| rac-[(2,3-Dioctadecyloxypropyl)(2-hydroxyethyl)]- | CLIP-1 | Cationic |
| dimethylammonium chloride | | |
| rac-[2(2,3-Dihexadecyloxypropyl- | CLIP-6 | Cationic |
| oxymethyloxy)ethyl]trimethylammoniun bromide | | |
| Ethyldimyristoylphosphatidylcholine | EDMPC | Cationic |
| 1,2-Distearyloxy-N,N-dimethyl-3-aminopropane | DSDMA | Cationic |
| 1,2-Dimyristoyl-trimethylammonium propane | DMTAP | Cationic |
| O,O′-Dimyristyl-N-lysyl aspartate | DMKE | Cationic |
| 1,2-Distearoyl-sn-glycero-3-ethylpho sphocholine | DSEPC | Cationic |
| N-Palmitoyl D-erythro-sphingosyl carbamoyl-spermine | CCS | Cationic |
| N-t-Butyl-N0-tetradecyl-3-tetradecylaminopropionamidine | diC14-amidine | Cationic |
| Octadecenolyoxy[ethyl-2-heptadecenyl-3 hydroxyethyl] | DOTIM | Cationic |
| imidazolinium chloride | | |
| N1-Cholesteryloxycarbonyl-3,7-diazanonane-1,9-diamine | CDAN | Cationic |
| 2-(3-[Bis(3-amino-propyl)-amino]propylamino)-N- | RPR209120 | Cationic |
| ditetradecylcarbamoylme-ethyl-acetamide | | |
| 1,2-dilinoleyloxy-3-dimethylaminopropane | DLinDMA | Cationic |
| 2,2-dilinoleyl-4-dimethylaminoethyl-[1,3]-dioxolane | DLin-KC2-DMA | Cationic |
| dilinoleyl-methyl-4-dimethylaminobutyrate | DLin-MC3-DMA | Cationic |

Table 6 lists exemplary polymers for use in gene transfer and/or nanoparticle formulations.

TABLE 6
Polymers Used for Gene Transfer

| Polymer | Abbreviation |
|---|---|
| Poly(ethylene)glycol | PEG |
| Polyethylenimine | PEI |
| Dithiobis (succinimidylpropionate) | DSP |
| Dimethyl-3,3′-dithiobispropionimidate | DTBP |
| Poly(ethylene imine)biscarbamate | PEIC |
| Poly(L-lysine) | PLL |
| Histidine modified PLL | |
| Poly(N-vinylpyrrolidone) | PVP |
| Poly(propylenimine) | PPI |
| Poly(amidoamine) | PAMAM |
| Poly(amidoethylenimine) | SS-PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | pHPMA |
| Poly (2-(dimethylamino)ethyl methacrylate) | pDMAEMA |
| Poly(2-aminoethyl propylene phosphate) | PPE-EA |
| Chitosan | |
| Galactosylated chitosan | |
| N-Dodacylated chitosan | |
| Histone | |
| Collagen | |
| Dextran-spermine | D-SPM |

Table 7 summarizes delivery methods for a polynucleotide encoding a Cas9 described herein.

TABLE 7

| | | Delivery into | | | Type of |
|---|---|---|---|---|---|
| | | Non-Dividing | Duration of | Genome | Molecule |
| Delivery | Vector/Mode | Cells | Expression | Integration | Delivered |
| Physical | (e.g., | YES | Transient | NO | Nucleic Acids |
| | electroporation, | | | | and Proteins |
| | particle gun, | | | | |
| | Calcium | | | | |
| | Phosphate | | | | |
| | transfection | | | | |
| Viral | Retrovirus | NO | Stable | YES | RNA |
| | Lentivirus | YES | Stable | YES/NO with | RNA |
| | | | | modification | |
| | Adenovirus | YES | Transient | NO | DNA |
| | Adeno- | YES | Stable | NO | DNA |
| | Associated | | | | |
| | Virus (AAV) | | | | |
| | Vaccinia Virus | YES | Very | NO | DNA |
| | | | Transient | | |
| | Herpes Simplex | YES | Stable | NO | DNA |
| | Virus | | | | |
| Non-Viral | Cationic | YES | Transient | Depends on | Nucleic Acids |
| | Liposomes | | | what is | and Proteins |
| | | | | delivered | |
| | Polymeric | YES | Transient | Depends on | Nucleic Acids |
| | Nanoparticles | | | what is | and Proteins |
| | | | | delivered | |
| Biological | Attenuated | YES | Transient | NO | Nucleic Acids |
| Non-Viral | Bacteria | | | | |
| Delivery | Engineered | YES | Transient | NO | Nucleic Acids |
| Vehicles | Bacteriophages | YES | Transient | NO | Nucleic Acids |
| | Mammalian | | | | |
| | Virus-like | | | | |
| | Particles | | | | |
| | Biological | YES | Transient | NO | Nucleic Acids |
| | liposomes: | | | | |
| | Erythrocyte | | | | |
| | Ghosts and | | | | |
| | Exosomes | | | | |

In another aspect, the delivery of genome editing system components or nucleic acids encoding such components, for example, a nucleic acid binding protein such as, for example, Cas9 or variants thereof, optionally fused to a polypeptide having biological activity (e.g., a nucleobase editor), and a gRNA targeting a genomic nucleic acid sequence of interest, may be accomplished by delivering a ribonucleoprotein (RNP) to cells. The RNP comprises the nucleic acid binding protein, e.g., Cas9, in complex with the targeting gRNA. RNPs may be delivered to cells using known methods, such as electroporation, nucleofection, or cationic lipid-mediated methods, for example, as reported by Zuris, J. A. et al., 2015, Nat. Biotechnology, 33(1):73-80. RNPs are advantageous for use in CRISPR base editing systems, particularly for cells that are difficult to transfect, such as primary cells. In addition, RNPs can also alleviate difficulties that may occur with protein expression in cells, especially when eukaryotic promoters, e.g., CMV or EF1A, which may be used in CRISPR plasmids, are not well-expressed. Advantageously, the use of RNPs does not require the delivery of foreign DNA into cells. Moreover, because an RNP comprising a nucleic acid binding protein and gRNA complex is degraded over time, the use of RNPs has the potential to limit off-target effects. In a manner similar to that for plasmid based techniques, RNPs can be used to deliver binding protein (e.g., Cas9 variants) and to direct homology directed repair (HDR).

A promoter used to drive the CRISPR system (e.g., including the Cas9 described herein) can include AAV ITR. This can be advantageous for eliminating the need for an additional promoter element, which can take up space in the vector. The additional space freed up can be used to drive the expression of additional elements, such as a guide nucleic acid or a selectable marker. ITR activity is relatively weak, so it can be used to reduce potential toxicity due to over expression of the chosen nuclease.

Any suitable promoter can be used to drive expression of the Cas9 and, where appropriate, the guide nucleic acid. For ubiquitous expression, promoters that can be used include CMV, CAG, CBh, PGK, SV40, Ferritin heavy or light chains, etc. For brain or other CNS cell expression, suitable promoters can include: SynapsinI for all neurons, CaMKIIalpha for excitatory neurons, GAD67 or GAD65 or VGAT for GABAergic neurons, etc. For liver cell expression, suitable promoters include the Albumin promoter. For lung cell expression, suitable promoters can include SP-B. For endothelial cells, suitable promoters can include ICAM. For hematopoietic cells suitable promoters can include IFNbeta or CD45. For Osteoblasts suitable promoters can include OG-2.

In some cases, a Cas9 of the present disclosure is of small enough size to allow separate promoters to drive expression of the base editor and a compatible guide nucleic acid within the same nucleic acid molecule. For instance, a vector or viral vector can comprise a first promoter operably linked to a nucleic acid encoding the base editor and a second promoter operably linked to the guide nucleic acid.

The promoter used to drive expression of a guide nucleic acid can include: Pol III promoters such as U6 or H1 Use of Pol II promoter and intronic cassettes to express gRNA Adeno Associated Virus (AAV).

A Cas9 described herein with or without one or more guide nucleic can be delivered using adeno associated virus (AAV), lentivirus, adenovirus or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For example, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus. For plasmid delivery, the route of administration, formulation and dose can be as in U.S. Pat. No. 5,846,946 and as in clinical studies involving plasmids. Doses can be based on or extrapolated to an average 70 kg individual (e.g. a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The viral vectors can be injected into the tissue of interest. For cell-type specific base editing, the expression of the base editor and optional guide nucleic acid can be driven by a cell-type specific promoter.

For in vivo delivery, AAV can be advantageous over other viral vectors. In some cases, AAV allows low toxicity, which can be due to the purification method not requiring ultra-centrifugation of cell particles that can activate the immune response. In some cases, AAV allows low probability of causing insertional mutagenesis because it doesn't integrate into the host genome.

AAV has a packaging limit of 4.5 or 4.75 Kb. Constructs larger than 4.5 or 4.75 Kb can lead to significantly reduced virus production. For example, SpCas9 is quite large, the gene itself is over 4.1 Kb, which makes it difficult for packing into AAV. Therefore, embodiments of the present disclosure include utilizing a disclosed Cas9 which is shorter in length than conventional Cas9.

An AAV can be AAV1, AAV2, AAV5 or any combination thereof. One can select the type of AAV with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. A tabulation of certain AAV serotypes as to these cells can be found in Grimm, D. et al, J. Virol. 82: 5887-5911 (2008)).

Lentiviruses are complex retroviruses that have the ability to infect and express their genes in both mitotic and post-mitotic cells. The most commonly known lentivirus is the human immunodeficiency virus (HIV), which uses the envelope glycoproteins of other viruses to target a broad range of cell types.

Lentiviruses can be prepared as follows. After cloning pCasES10 (which contains a lentiviral transfer plasmid backbone), HEK293FT at low passage (p=5) were seeded in a T-75 flask to 50% confluence the day before transfection in DMEM with 10% fetal bovine serum and without antibiotics. After 20 hours, media is changed to OptiMEM (serum-free) media and transfection was done 4 hours later. Cells are transfected with 10 μg of lentiviral transfer plasmid (pCasES10) and the following packaging plasmids: 5 μg of pMD2.G (VSV-g pseudotype), and 7.5 μg of psPAX2 (gag/pol/rev/tat). Transfection can be done in 4 mL OptiMEM with a cationic lipid delivery agent (50 μl Lipofectamine 2000 and 100 ul Plus reagent). After 6 hours, the media is changed to antibiotic-free DMEM with 10% fetal bovine serum. These methods use serum during cell culture, but serum-free methods are preferred.

Lentivirus can be purified as follows. Viral supernatants are harvested after 48 hours. Supernatants are first cleared of debris and filtered through a 0.45 μm low protein binding (PVDF) filter. They are then spun in an ultracentrifuge for 2 hours at 24,000 rpm. Viral pellets are resuspended in 50 μl of DMEM overnight at 4° C. They are then aliquoted and immediately frozen at −80° C.

In another embodiment, minimal non-primate lentiviral vectors based on the equine infectious anemia virus (EIAV) are also contemplated. In another embodiment, RetinoStat®, an equine infectious anemia virus-based lentiviral gene therapy vector that expresses angiostatic proteins endostatin and angiostatin that is contemplated to be delivered via a subretinal injection. In another embodiment, use of self-inactivating lentiviral vectors is contemplated.

Any RNA of the systems, for example a guide RNA or a Cas9-encoding mRNA, can be delivered in the form of RNA. Cas9 encoding mRNA can be generated using in vitro transcription. For example, Cas9 mRNA can be synthesized using a PCR cassette containing the following elements: T7 promoter, optional kozak sequence (GCCACC), nuclease sequence, and 3′ UTR such as a 3′ UTR from beta globin-polyA tail. The cassette can be used for transcription by T7 polymerase. Guide polynucleotides (e.g., gRNA) can also be transcribed using in vitro transcription from a cassette containing a T7 promoter, followed by the sequence “GG”, and guide polynucleotide sequence.

To enhance expression and reduce possible toxicity, the Cas9 sequence and/or the guide nucleic acid can be modified to include one or more modified nucleoside e.g. using pseudo-U or 5-Methyl-C.

The disclosure in some embodiments comprehends a method of modifying a cell or organism. The cell can be a prokaryotic cell or a eukaryotic cell. The cell can be a mammalian cell. The mammalian cell many be a non-human primate, bovine, porcine, rodent or mouse cell. The modification introduced to the cell by the base editors, compositions and methods of the present disclosure can be such that the cell and progeny of the cell are altered for improved production of biologic products such as an antibody, starch, alcohol or other desired cellular output. The modification introduced to the cell by the methods of the present disclosure can be such that the cell and progeny of the cell include an alteration that changes the biologic product produced.

The system can comprise one or more different vectors. In an aspect, the Cas9 is codon optimized for expression the desired cell type, preferentially a eukaryotic cell, preferably a mammalian cell or a human cell.

In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/(visited Jul. 9, 2002), and these tables can be adapted in a number of ways. See, Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding an engineered nuclease correspond to the most frequently used codon for a particular amino acid.

Packaging cells are typically used to form virus particles that are capable of infecting a host cell. Such cells include 293 cells, which package adenovirus, and psi.2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA can be packaged in a cell line, which contains a helper plasmid encoding the other AAV genes, namely rep and cap, but lacking ITR sequences. The cell line can also be infected with adenovirus as a helper. The helper virus can promote replication of the AAV vector and expression of AAV genes from the helper plasmid. The helper plasmid in some cases is not packaged in significant amounts due to a lack of ITR sequences. Contamination with adenovirus can be reduced by, e.g., heat treatment to which adenovirus is more sensitive than AAV.

### Pharmaceutical Compositions

Other aspects of the present disclosure relate to pharmaceutical compositions comprising CRISPR system (e.g., including Cas9 disclosed herein). The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents (e.g., for specific delivery, increasing half-life, or other therapeutic compounds).

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.).

Some nonlimiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient,” “carrier,” “pharmaceutically acceptable carrier,” “vehicle,” or the like are used interchangeably herein.

Pharmaceutical compositions can comprise one or more pH buffering compounds to maintain the pH of the formulation at a predetermined level that reflects physiological pH, such as in the range of about 5.0 to about 8.0. The pH buffering compound used in the aqueous liquid formulation can be an amino acid or mixture of amino acids, such as histidine or a mixture of amino acids such as histidine and glycine. Alternatively, the pH buffering compound is preferably an agent which maintains the pH of the formulation at a predetermined level, such as in the range of about 5.0 to about 8.0, and which does not chelate calcium ions. Illustrative examples of such pH buffering compounds include, but are not limited to, imidazole and acetate ions. The pH buffering compound may be present in any amount suitable to maintain the pH of the formulation at a predetermined level.

Pharmaceutical compositions can also contain one or more osmotic modulating agents, i.e., a compound that modulates the osmotic properties (e.g, tonicity, osmolality, and/or osmotic pressure) of the formulation to a level that is acceptable to the blood stream and blood cells of recipient individuals. The osmotic modulating agent can be an agent that does not chelate calcium ions. The osmotic modulating agent can be any compound known or available to those skilled in the art that modulates the osmotic properties of the formulation. One skilled in the art may empirically determine the suitability of a given osmotic modulating agent for use in the inventive formulation. Illustrative examples of suitable types of osmotic modulating agents include, but are not limited to: salts, such as sodium chloride and sodium acetate; sugars, such as sucrose, dextrose, and mannitol; amino acids, such as glycine; and mixtures of one or more of these agents and/or types of agents. The osmotic modulating agent(s) may be present in any concentration sufficient to modulate the osmotic properties of the formulation.

In some embodiments, the pharmaceutical composition is formulated for delivery to a subject, e.g., for gene editing. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site. In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In other embodiments, the pharmaceutical composition described herein is delivered in a controlled release system. In one embodiment, a pump can be used (See, e.g., Langer, 1990, Science 249: 1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61.

See also Levy et al., 1985, Science 228: 190; During et al., 1989, Ann. Neurol. 25:351; Howard et ah, 1989, J. Neurosurg. 71: 105.) Other controlled release systems are discussed, for example, in Langer, supra.

In some embodiments, the pharmaceutical composition is formulated in accordance with routine procedures as a composition adapted for intravenous or subcutaneous administration to a subject, e.g., a human. In some embodiments, pharmaceutical composition for administration by injection are solutions in sterile isotonic use as solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

A pharmaceutical composition for systemic administration can be a liquid, e.g., sterile saline, lactated Ringer's or Hank's solution. In addition, the pharmaceutical composition can be in solid forms and re-dissolved or suspended immediately prior to use. Lyophilized forms are also contemplated. The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Compounds can be entrapped in “stabilized plasmid-lipid particles” (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et ah, Gene Ther. 1999, 6: 1438-47). Positively charged lipids such as N-[1-(2,3-dioleoyloxi)propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757; each of which is incorporated herein by reference.

The pharmaceutical composition described herein can be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the pharmaceutical composition can be provided as a pharmaceutical kit comprising (a) a container containing a compound of the invention in lyophilized form and (b) a second container containing a pharmaceutically acceptable diluent (e.g., sterile used for reconstitution or dilution of the lyophilized compound of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers can be formed from a variety of materials such as glass or plastic. In some embodiments, the container holds a composition that is effective for treating a disease described herein and can have a sterile access port. For example, the container can be an intravenous solution bag or a vial having a stopper pierceable by a hypodermic injection needle. The active agent in the composition is a compound of the invention. In some embodiments, the label on or associated with the container indicates that the composition is used for treating the disease of choice. The article of manufacture can further comprise a second container comprising a pharmaceutically-acceptable buffer, such as phosphate-buffered saline, Ringer's solution, or dextrose solution. It can further include other materials desirable from a commercial and user standpoint, including other buffers, diluents, filters, needles, syringes, and package inserts with instructions for use.

In some embodiments, the CRISPR system (e.g., including the Cas9 described herein) are provided as part of a pharmaceutical composition. In some embodiments, the pharmaceutical composition comprises any of the fusion proteins provided herein (e.g., including the nucleobase editor described herein comprising LubCas9). In some embodiments, the pharmaceutical composition comprises any of the complexes provided herein. In some embodiments, the pharmaceutical composition comprises a ribonucleoprotein complex comprising an RNA-guided nuclease (e.g., Cas9) that forms a complex with a gRNA and a cationic lipid. In some embodiments pharmaceutical composition comprises a gRNA, a nucleic acid programmable DNA binding protein, a cationic lipid, and a pharmaceutically acceptable excipient. Pharmaceutical compositions can optionally comprise one or more additional therapeutically active substances.

### Kits

In one aspect, the invention provides kits containing any one or more of the elements disclosed in the above methods and compositions. In some embodiments, the kit comprises a vector system and instructions for using the kit. In some embodiments, the vector system comprises one or more insertion sites for inserting a guide sequence, wherein when expressed, the guide sequence directs sequence-specific binding of a CRISPR complex to a target sequence in a eukaryotic cell, wherein the CRISPR complex comprises a CRISPR enzyme complexed with (1) the guide sequence that is hybridized to the target sequence, and (2) a sequence that is hybridized to the tracrRNA sequence; and/or (b) a second regulatory element operably linked to an enzyme-coding sequence encoding said CRISPR enzyme comprising a nuclear localization sequence. Elements may be provide individually or in combinations, and may be provided in any suitable container, such as a vial, a bottle, or a tube. In some embodiments, the kit includes instructions in one or more languages, for example in more than one language.

In some embodiments, the kit comprises a nucleobase editor. For example, in some embodiments, the kit includes a nucleobase editor comprising the Cas9 enzymes (ScoCas9, Seq2Cas9, EhiCas9, SeqCas9, SsiCas9, SinCas9, SsaCas9, Ssc2Cas9, Sor2Cas9, SorCas9, SwaCas9, SscCas9, SgaCas9, LkuCas9 and SsuCas9) described herein.

In some embodiments, a kit comprises one or more reagents for use in a process utilizing one or more of the elements described herein. Reagents may be provided in any suitable container. For example, a kit may provide one or more reaction or storage buffers. Reagents may be provided in a form that is usable in a particular assay, or in a form that requires addition of one or more other components before use (e.g. in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10. In some embodiments, the kit comprises one or more oligonucleotides corresponding to a guide sequence for insertion into a vector so as to operably link the guide sequence and a regulatory element. In some embodiments, the kit comprises a homologous recombination template polynucleotide.

All publications, patent applications, patents, and other references mentioned herein are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

### EXAMPLES

The following examples describe some of the preferred modes of making and practicing the present invention. However, it should be understood that these examples are for illustrative purposes only and are not meant to limit the scope of the invention.

### Example 1. Screening for Novel Cas9 Enzymes, Discovery and Optimization of Novel Cas9 Enzymes

This example describes a screen for the discovery of novel Cas9 enzymes. As described herein, using this screen novel Cas9 enzymes from Streptococcus equinus ATCC 33317, Enterococcus hirae strain F1129E, Streptococcus equinus strain AG46, Staphylococcus simulans strain 19, Streptococcus intermedius B196 strain G1552, Streptococcus sanguinis SK330, Streptococcus sp. C150, Streptococcus oralis subsp. oralis strain RH_1735_08, Streptococcus oralis SK313, Staphylococcus warneri strain 691, Staphylococcus stiuri strain SNUC 2430, Streptococcus gallolyticus strain AM24-4, Lactobacillus kullabergensis strain Biut2, and Streptococcus suis strain LSS83 bacteria were isolated and optimized.

In a search to discover new Cas9 enzymes which recognize novel PAM sequences, a bioinformatics screen was used to search for additional enzymes to expand CRISPR's targeting range. The screen utilized seed sequences of Cas9 from Lachnospira spp. LubCas9 or Streptococcus constellatus ScoCas9. Bioinformatics was carried out using the tblastn variant of BLAST with an e-value threshold of 1e-6 for considering BLAST hits. Briefly, loci selected for testing were loci that remained intact in the presence of Cas9 proteins from other species. Loci were selected that had greater than three spacers within the CRISPR array and greater than 1 kb endogenous sequence 5′ of Cas9 and greater than 300 nt 3′ of the CRISPR array. Using this approach, novel Cas9 enzymes were identified from different bacterial species and codon optimized for expression in human cells. The novel engineered Cas9 enzymes were then recombinantly produced and tested.

### Example 2. Identifying 3′ PAM Consensus Motif for Novel Cas9 Enzymes from Streptococcus Equinus ATCC 33317, Enterococcus hirae Strain F1129E, Streptococcus equinus Strain AG46, Staphylococcus simulans Strain 19, Streptococcus intermedius B196 Strain G1552, Streptococcus sanguinis SK330, Streptococcus sp. C150, Streptococcus oralis Subsp. Oralis Strain RH 1735 08, Streptococcus oralis SK313, Staphylococcus warneri Strain 691, Staphylococcus stiuri Strain SNUC 2430, Streptococcus Gallolvticus Strain AM24-4, Lactobacillus kullabergensis Strain Biut2, and Streptococcus suis Strain LSS83 Bacteria

This example illustrates the identification of the protospacer adjacent motif (PAM) sequence for human codon-optimized Cas9 originally isolated from from Streptococcus equinus ATCC 33317, Enterococcus hirae strain F1129E, Streptococcus equinus strain AG46, Staphylococcus simulans strain 19, Streptococcus intermedius B196 strain G1552, Streptococcus sanguinis SK330, Streptococcus sp. C150, Streptococcus oralis subsp. oralis strain RH_1735_08, Streptococcus oralis SK313, Staphylococcus warneri strain 691, Staphylococcus stiuri strain SNUC 2430, Streptococcus gallolyticus strain AM24-4, Lactobacillus kullabergensis strain Biut2, and Streptococcus suis strain LSS83 bacteria.

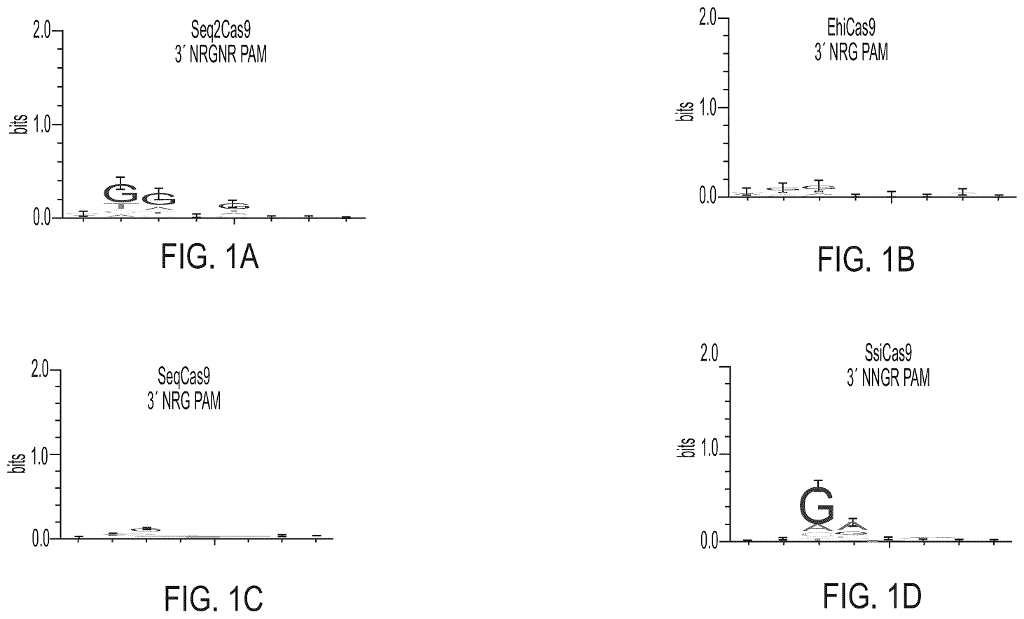

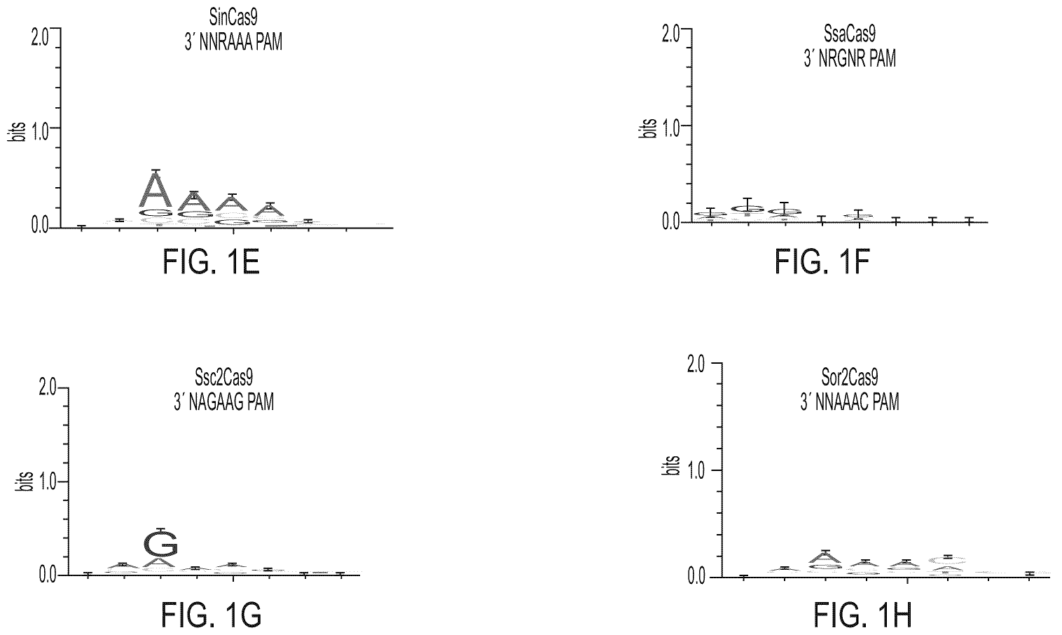

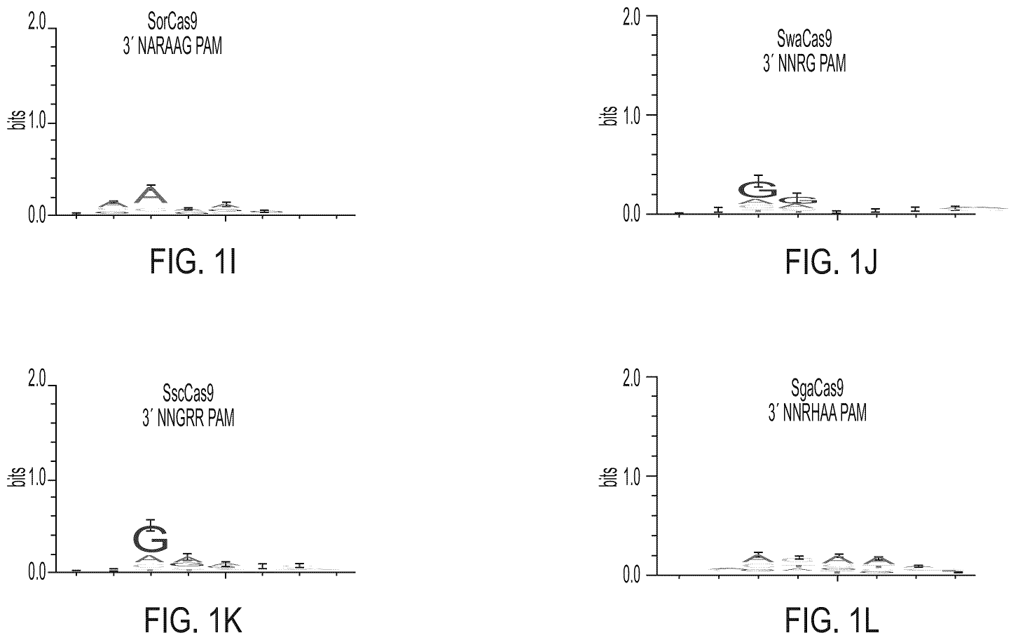

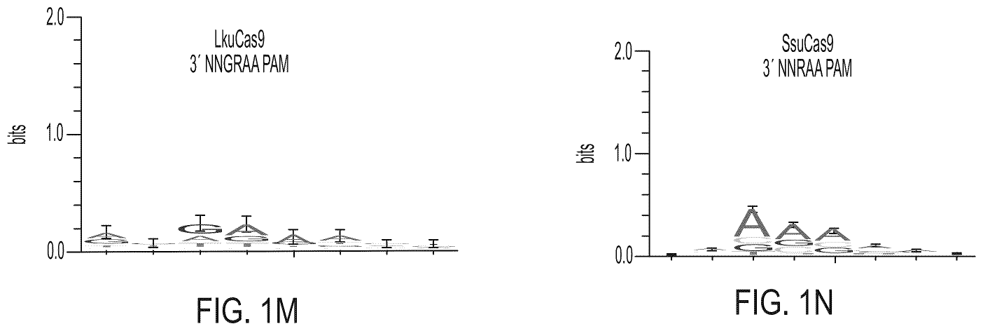

The human, codon-optimized Cas9 was tested for its recognition of a PAM sequence using an in vitro PAM identification assay. A library of plasmids bearing randomized PAM sequences were incubated with Cas9 isolated from different bacteria. Uncleaved plasmid was purified and sequenced to identify specific PAM motifs that were cleaved. For example, Cas9 engineered from Streptococcus equinus ATCC 33317 recognizes the consensus PAM sequence 5′-NRGNR-3′ (FIG. 1A), Enterococcus hirae strain F1129E recognizes the consensus PAM sequence 5′-NRG-3′ (FIG. 1), Streptococcus equinus strain AG46 (FIG. 1C), Staphylococcus warneri strain 691 (FIG. 1J), and Staphylococcus sciuri strain SNUC 2430 (FIG. 1K) recognize the consensus PAM sequence 5′-NNGR-3′, Staphylococcus simulans strain 19 recognizes the consensus PAM sequence 5′-NNGRRT-3′ (FIG. 1D), Streptococcus intermedius B196 strain G1552 recognizes the consensus PAM sequence 5′-NNAAAA-3′ (FIG. 1E), Streptococcus sanguinis SK330 recognizes the consensus PAM sequence 5′-NGGNG-3′ (FIG. 1F), Streptococcus sp. C150 recognizes the consensus PAM sequence 5′-NNGNRG-3′ (FIG. 1G), Streptococcus oralis subsp. oralis strain RH_1735_08 recognizes the consensus PAM sequence 5′-NNAAAC-3′ (FIG. 1H), Streptococcus oralis SK313 recognizes the consensus PAM sequence 5′-NNRAAG-3′ (FIG. 1I), Streptococcus gallolyticus strain AM24-4 recognizes the consensus PAM sequence 5′-NNAYAA-3′ (FIG. 1L), Lactobacillus kullabergensis strain Biut2 recognizes the consensus PAM sequence 5′-NNGAAA-3′ (FIG. 1M), and Streptococcus suis strain recognizes the consensus PAM sequence 5′-NNAAA-3′ (FIG. 1N) (H=A, C or T; R=A or G).

### Example 3. Predicting RNA Folding Structure of sgRNA for Novel Cas9 Enzymes from Streptococcus constellatus, Sharpea spp. isolate RUG017, Veillonella parvula, Ezakiella peruensis, Lactobacillus fermentum strain AF15-40LB and Peptoniphilus sp. Marseille-P3761 bacteria

This example demonstrates the predicted RNA folding structure of exemplary sgRNA comprising crRNA and tracrRNA for use with novel Cas9 enzymes.

Small RNA sequencing was carried out on RNA derived from an E. coli strain heterologously expressing Cas9 Crispr loci. Briefly, RNA was isolated from stationary phase bacteria by first resuspending the E. coli in Trizol, then homogenizing the bacteria with zirconia/silica beads in a homogenizer for three 1 min cycles. Total RNA was purified from homogenized samples, DNAse treated and 3′ dephosphorylated with T4 polynucleotide kinase and rRNA was removed. RNA libraries were prepared from rRNA-depleted RNA, and size selected for small RNA.

For RNA sequencing, transcripts were poly-A tailed with E. coli Poly (A) polymerase, ligated with 5′ RNA adapters using T4 RNA ligase 1 and reverse transcribed, followed by PCR amplification of cDNA with barcoded primers, and sequencing on a MiSeq. Reads from each sample were identified on the basis of their associated barcode and aligned to a reference sequence using BWA. Paired-end alignments were used to extract transcript sequences using Picard tools and the sequences were analyzed using Geneious software.

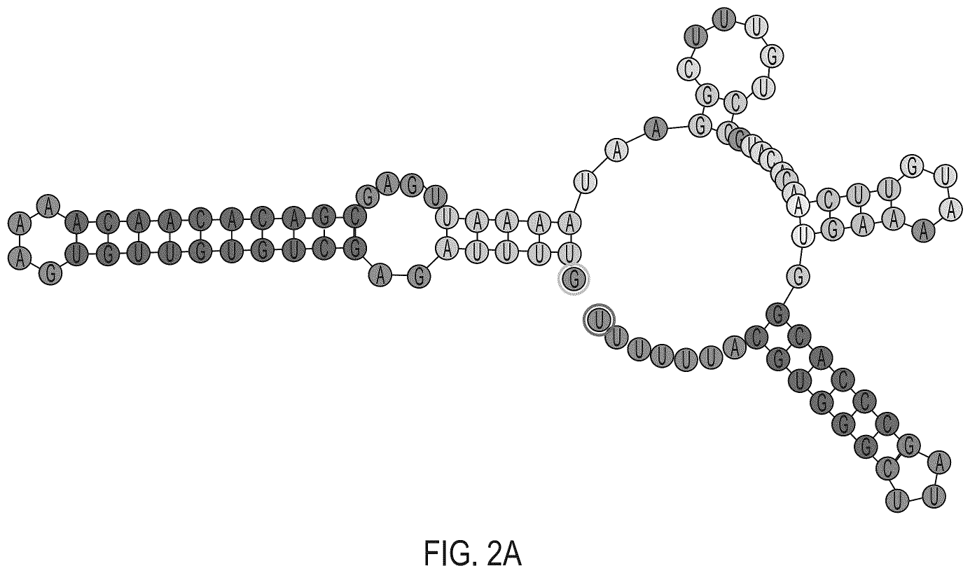

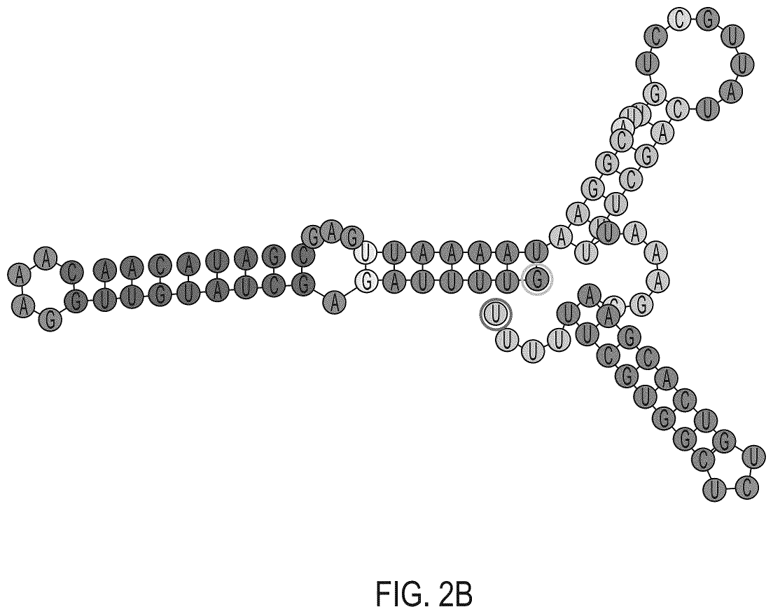

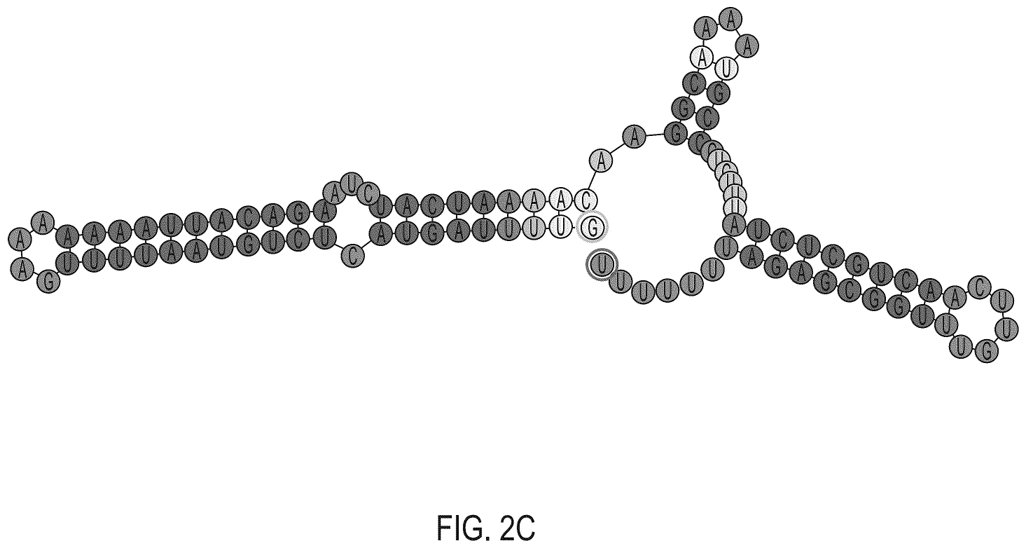

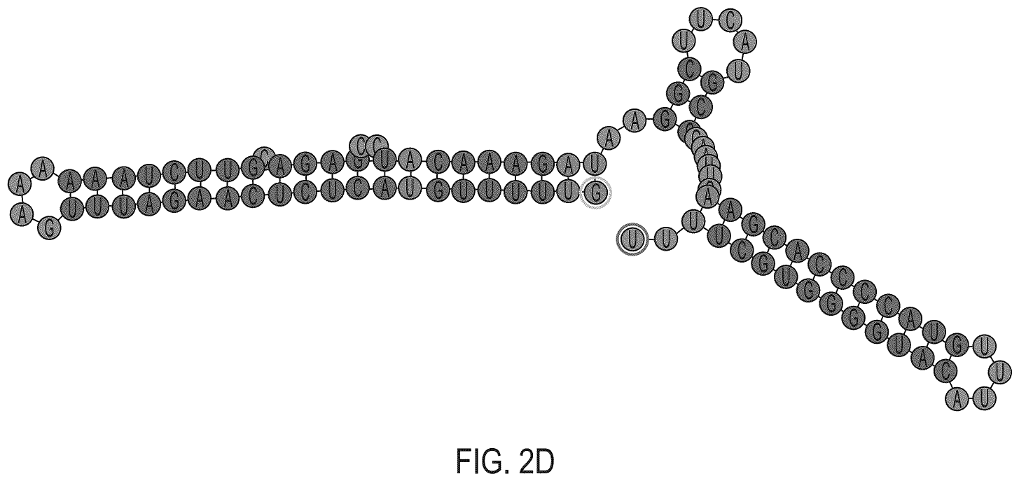

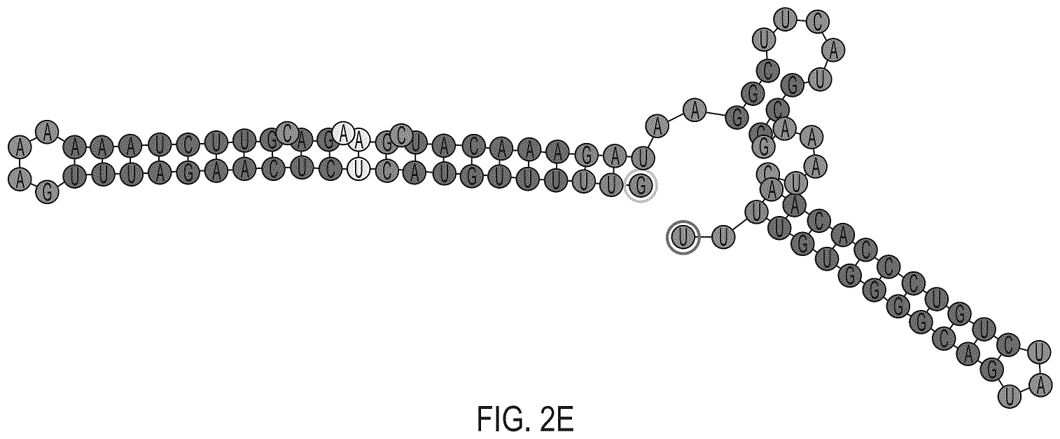

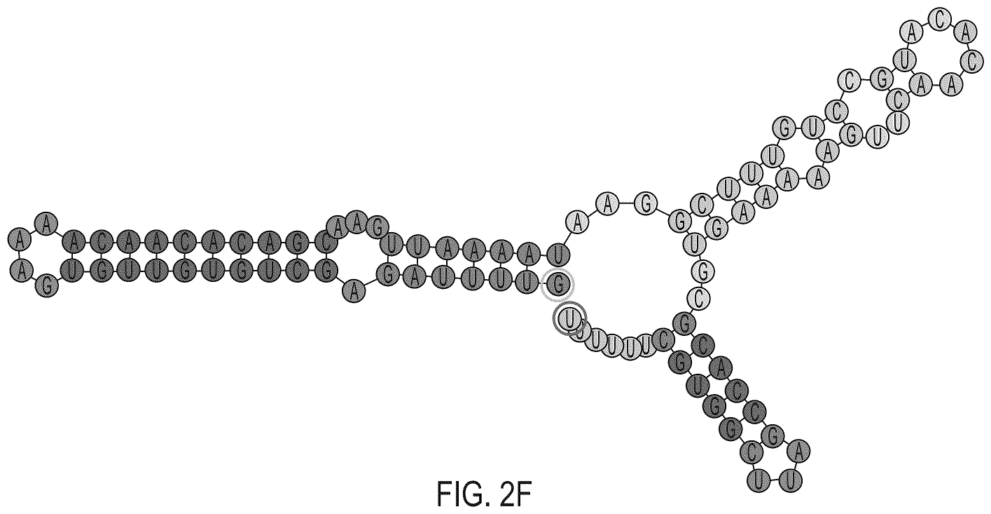

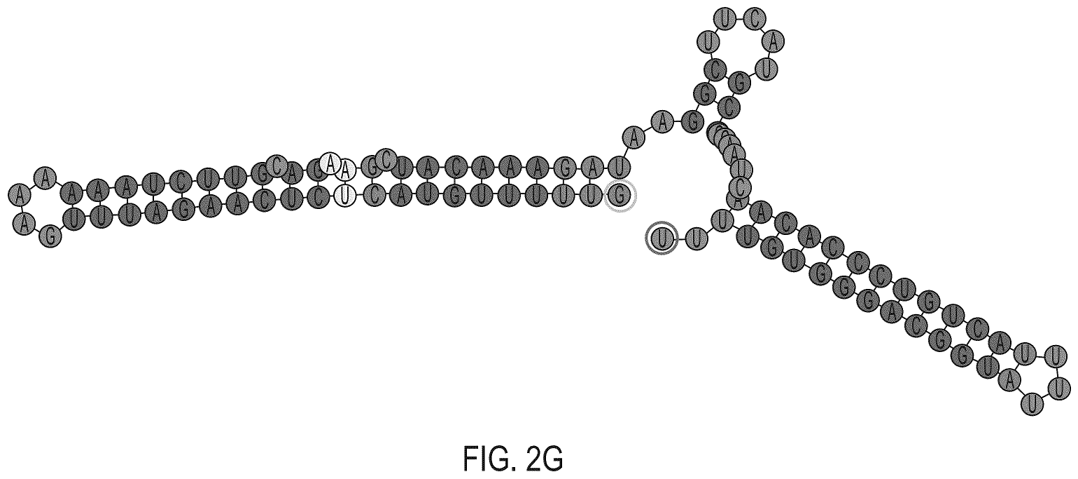

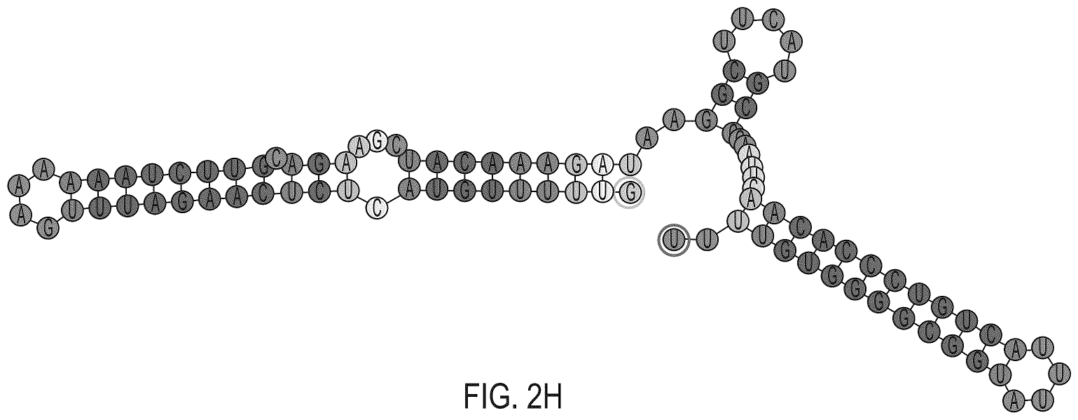

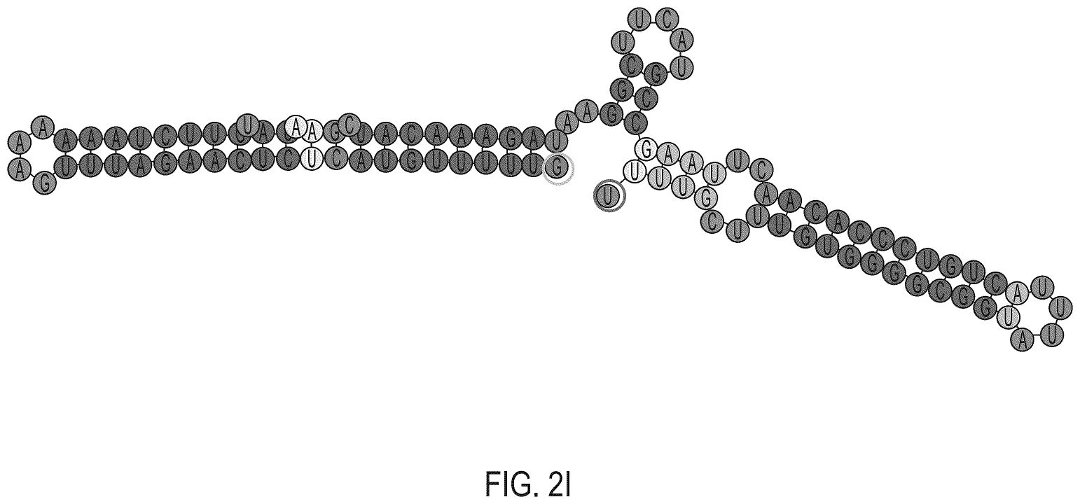

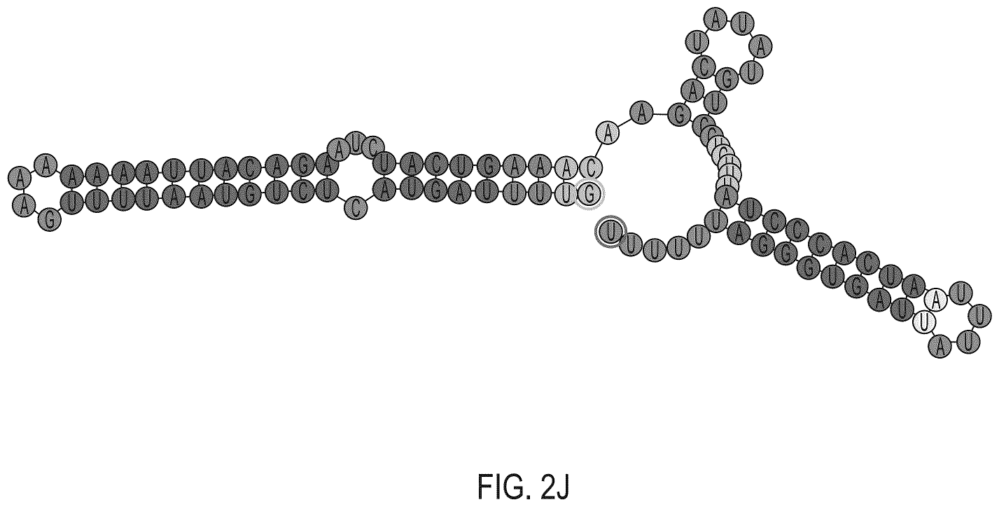

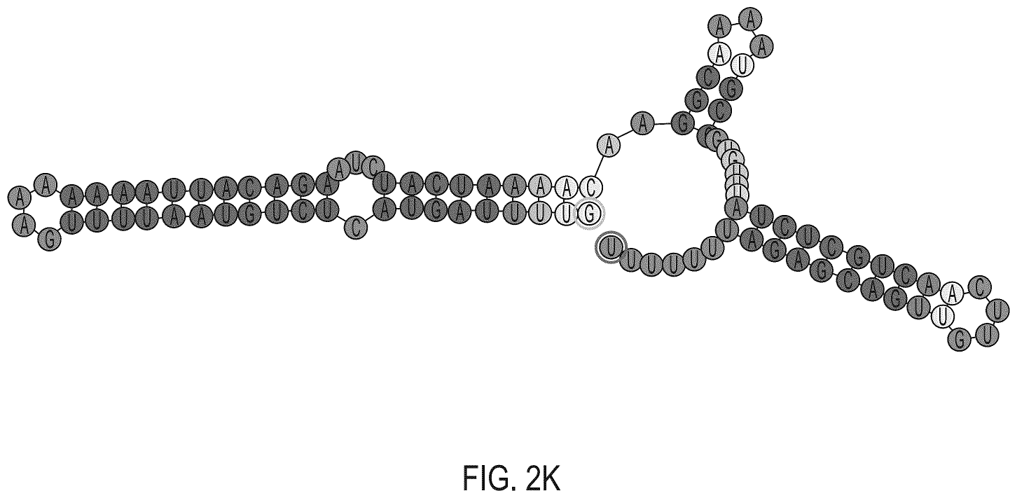

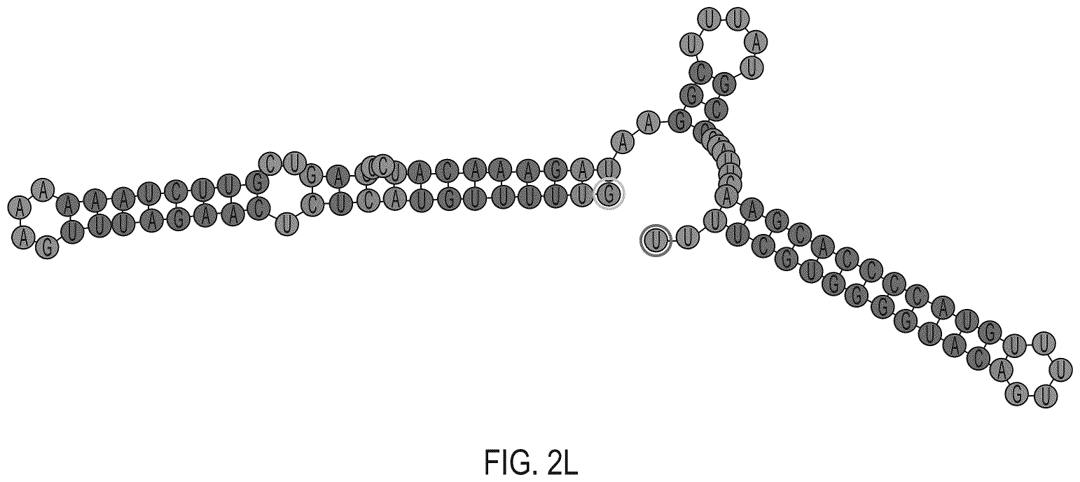

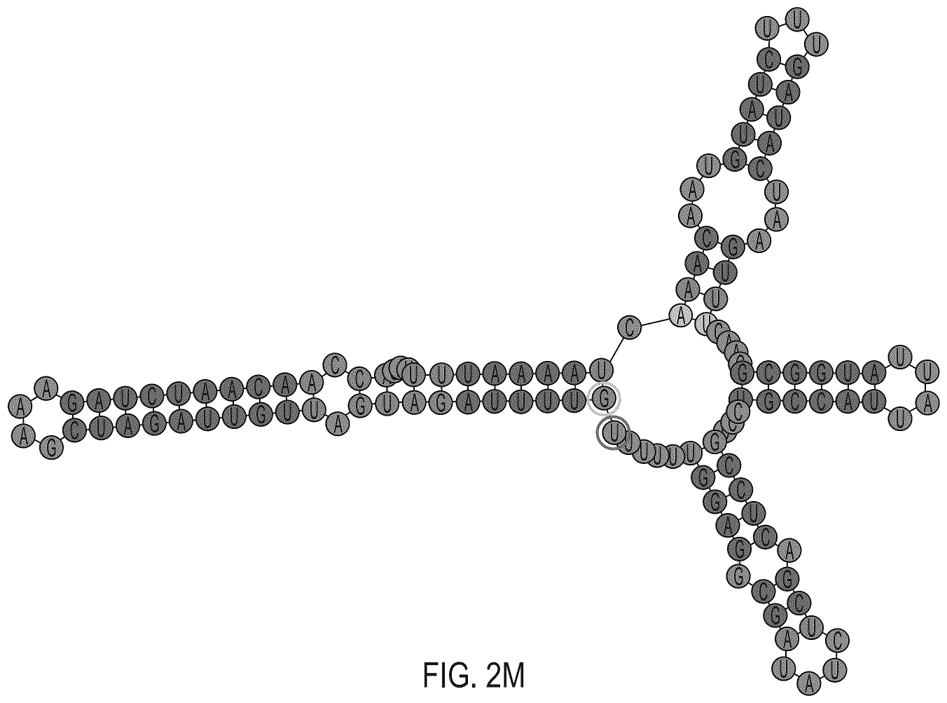

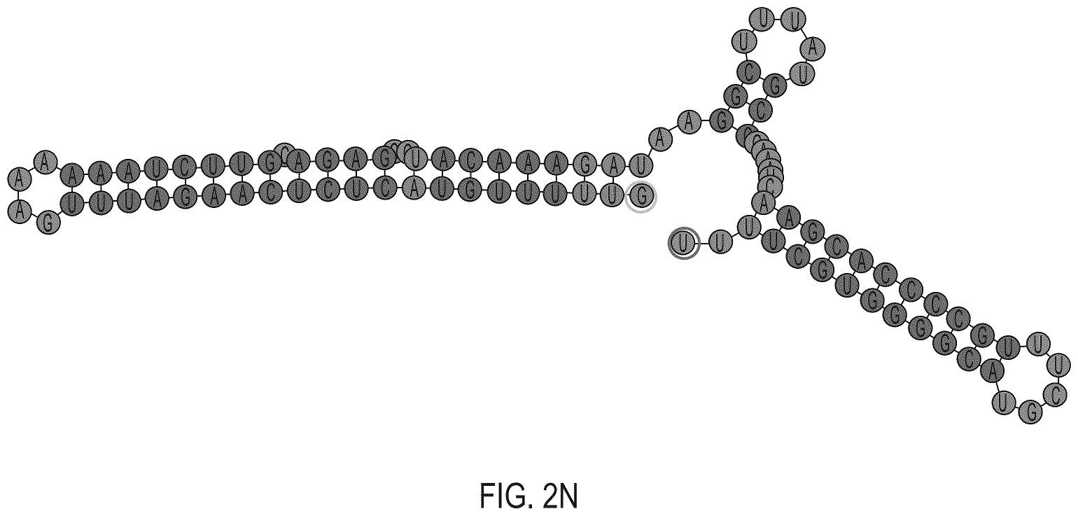

RNA folding was based on prediction from Geneious 11.1.2 software. The single sgRNA transcript fuses the crRNA to tracrRNA mimicking the dual RNA structure required to guide site-specific Cas9 activity. The predicted RNA folding structure for the chimeric sgRNA for use with Seq2Cas9 from Streptococcus equinus ATCC 33317 is shown in FIG. 2A, sgRNA for use with EhiCas9 from Enterococcus hirae strain F1129E is shown in FIG. 2B, sgRNA for use with SeqCas9 from Streptococcus equinus strain AG46 is shown in FIG. 2C, sgRNA for use with SsiCas9 from Staphylococcus simulans strain 19 is shown in FIG. 2D, sgRNA for use with SinCas9 from Streptococcus intermedius B196 strain G1552 is shown in FIG. 2E, sgRNA for use with SsaCas9 from Streptococcus sanguinis SK330 is shown in FIG. 2F, sgRNA for use with Ssc2Cas9 from Streptococcus sp. C150 is shown in FIG. 2G, sgRNA for use with Sor2Cas9 from Streptococcus oralis subsp. oralis strain RH_1735_08 is shown in FIG. 2H, sgRNA for use with Sor2Cas9 from Streptococcus oralis subsp. oralis strain RH_1735_08 is shown in FIG. 2I, sgRNA for use with SwaCas9 from Staphylococcus warneri strain 691 is shown in FIG. 2J, sgRNA for use with SsiCas9 from Staphylococcus sciuri strain SNUC 2430 is shown in FIG. 2K, sgRNA for use with SgaCas9 from Streptococcus gallolyticus strain AM24-4 is shown in FIG. 2L, sgRNA for use with LkuCas9 from Lactobacillus kullabergensis strain Biut2 is shown in FIG. 2M, and SsuCas9 from Streptococcus suis strain LSS83 is shown in FIG. 2N.

### Example 4. Base Editing by Cas9 Enzyme with an N-Terminal Fusion of an Adenine Base Editor (ABE) or a Cytidine Base Editor (CBE)

This example illustrates base conversion efficiency of a Cas9 enzyme fused to an adenine base editor (ABE), or to a cytidine base editor (CBE).

Briefly, 25,000 HEK293T cells were plated per 96-well. 100 ng of Cas9 expression plasmid and 100 ng of guide expression plasmid were transfected 24 h after plating. Cells were harvested 5 days after transfection and DNA was extracted.

Deep sequencing was carried out to characterize A-to-G conversion in the HEK293T cells. Exemplary targets were amplified using a two-round PCR region to add Illumina adapters as well as unique barcodes to the target amplicons. PCR products were run on a 2% gel and gel extracted. Samples were pooled, quantified and cDNA libraries were prepared and sequenced on MiSeq. The percent A-to-G conversion was determined by deep sequencing for the N-terminal as well as the C-terminal TadA8 fusion constructs.

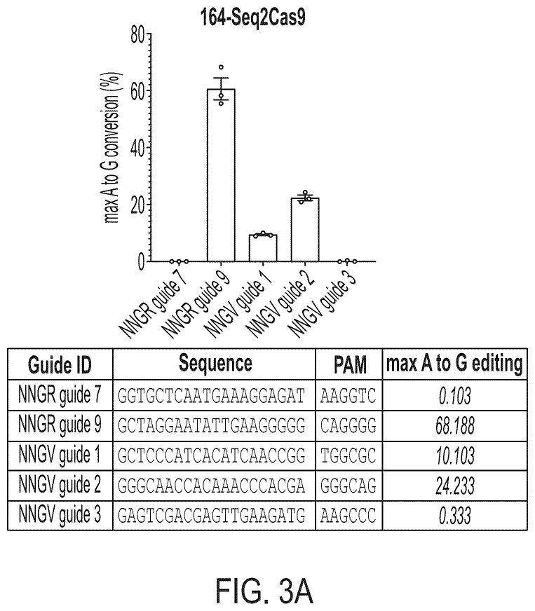

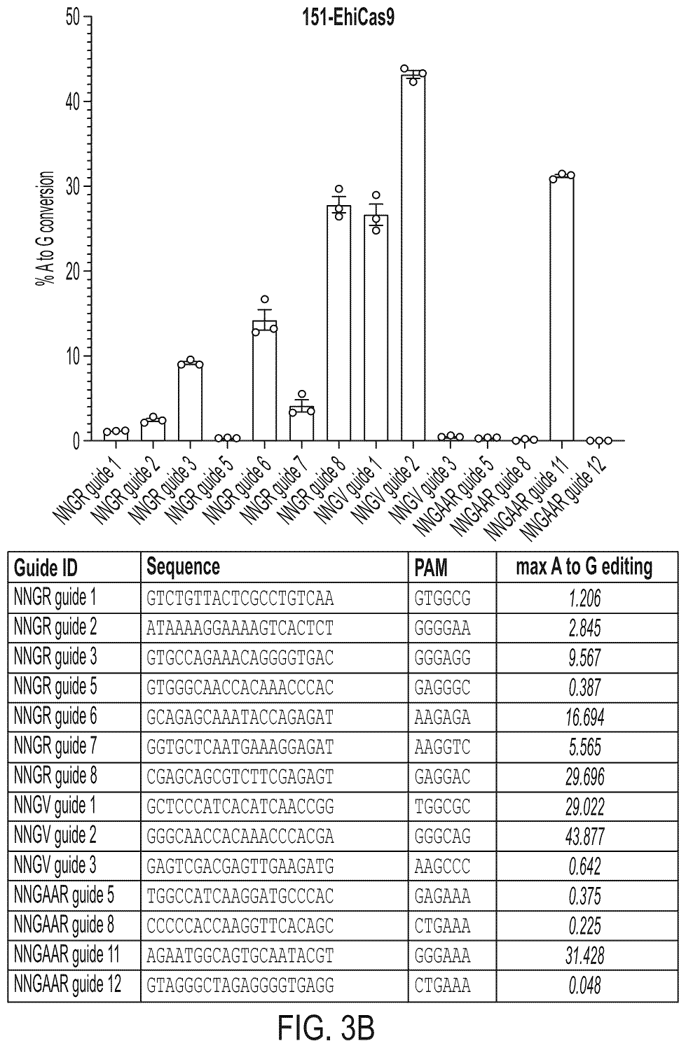

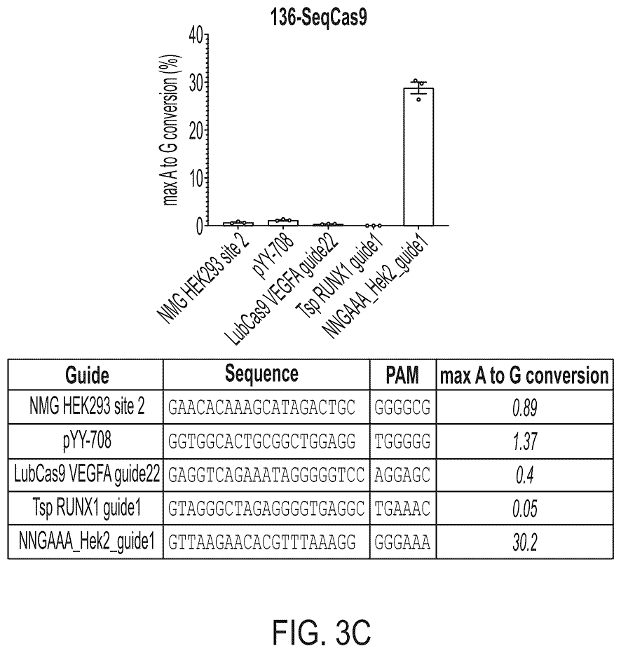

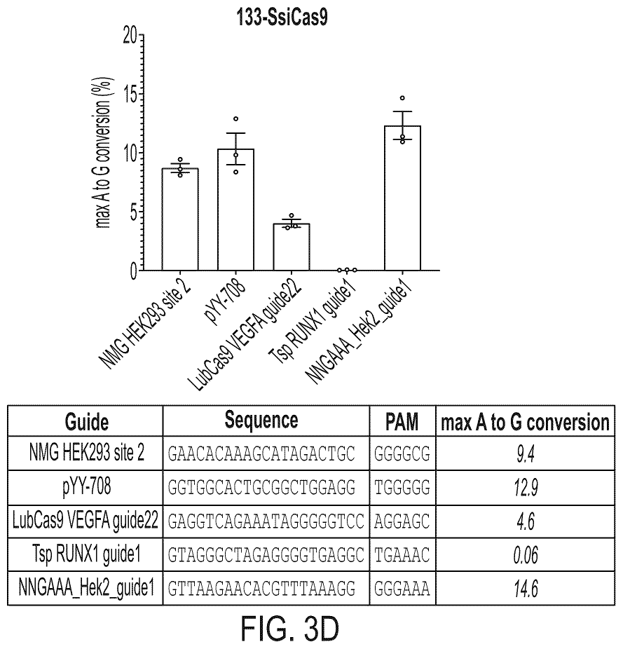

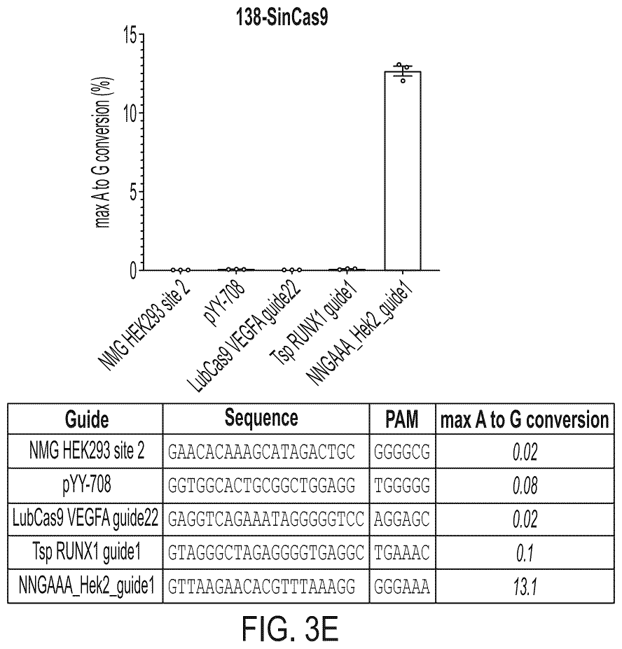

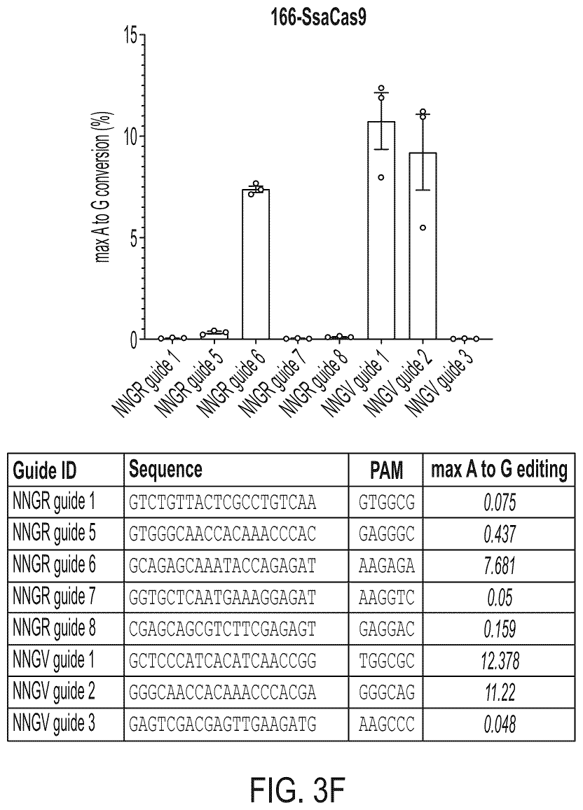

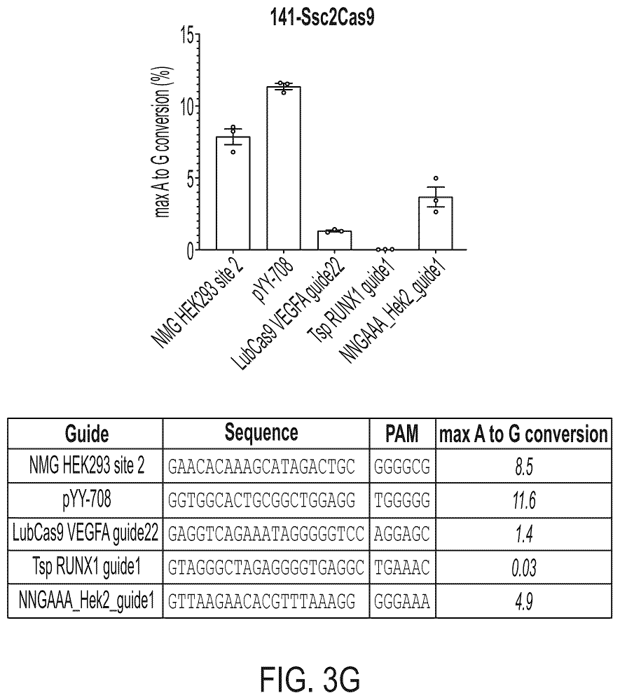

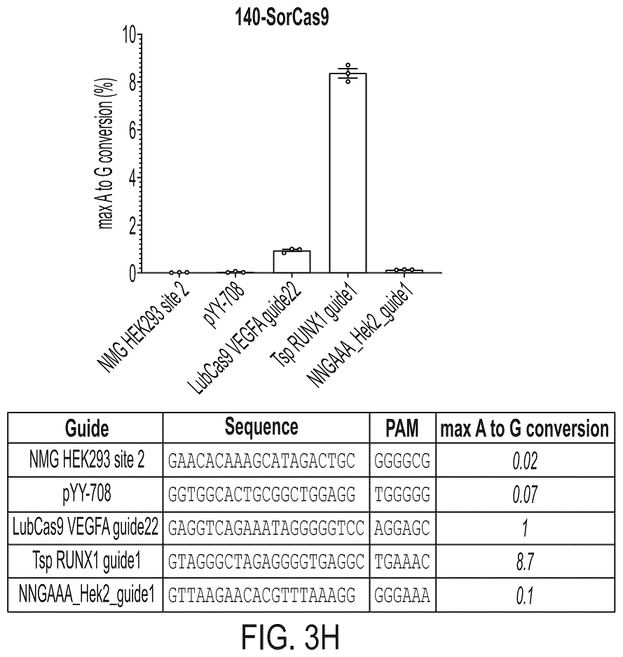

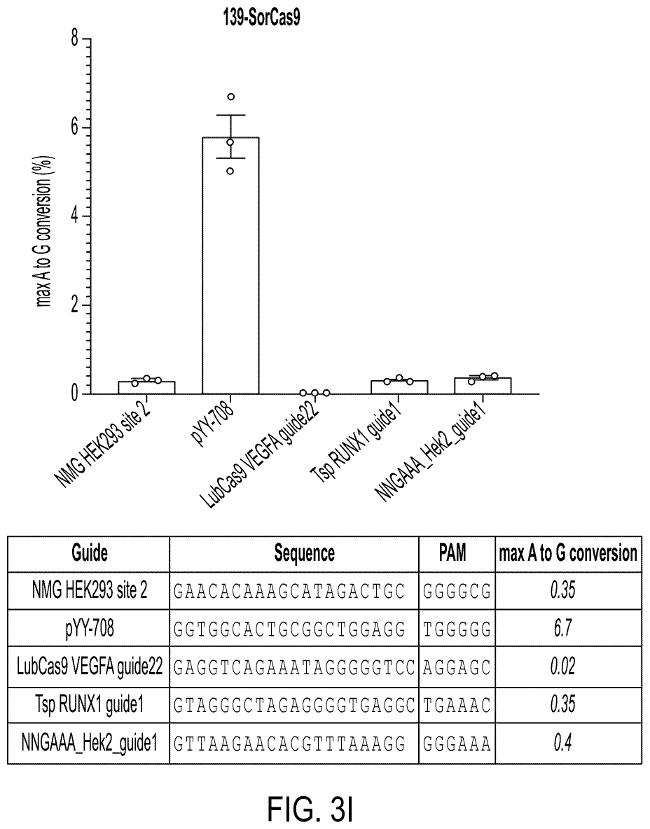

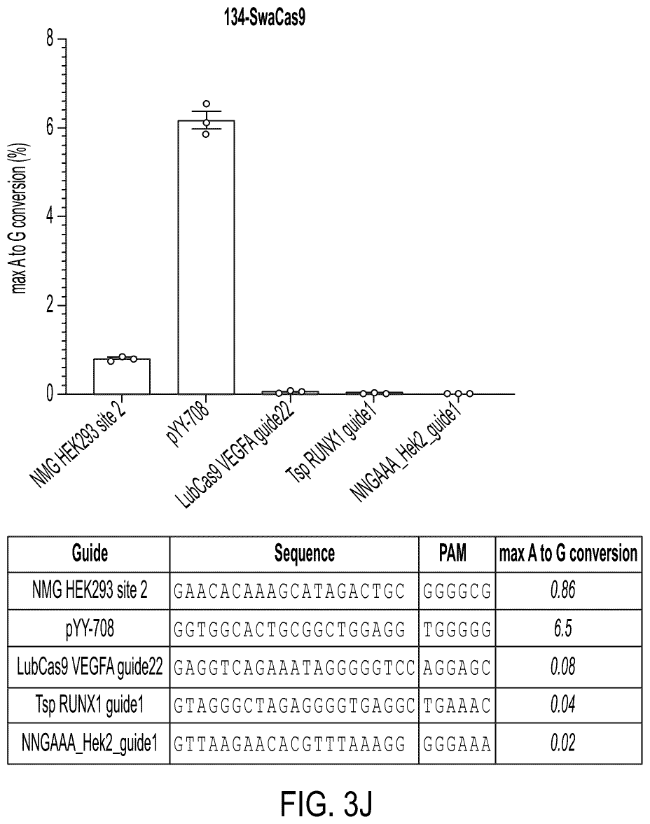

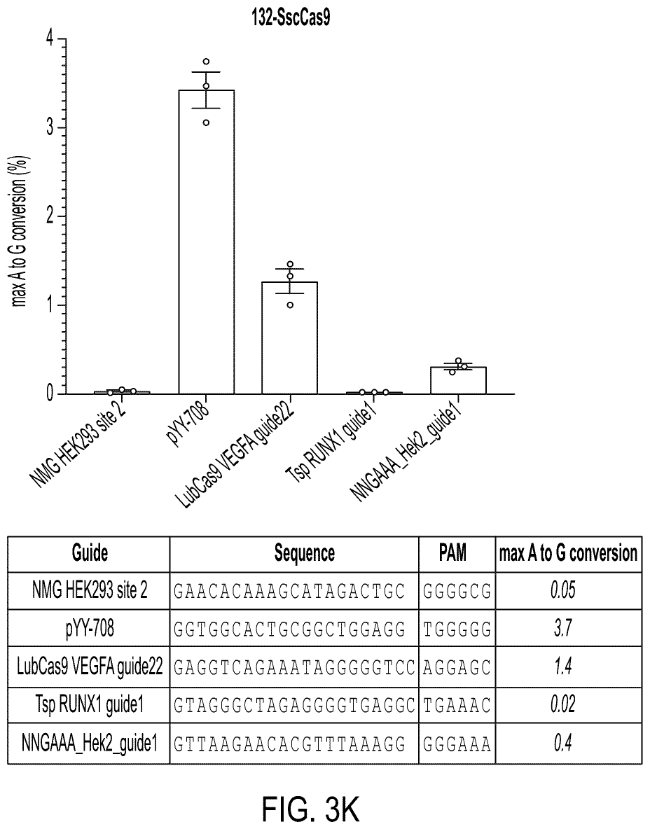

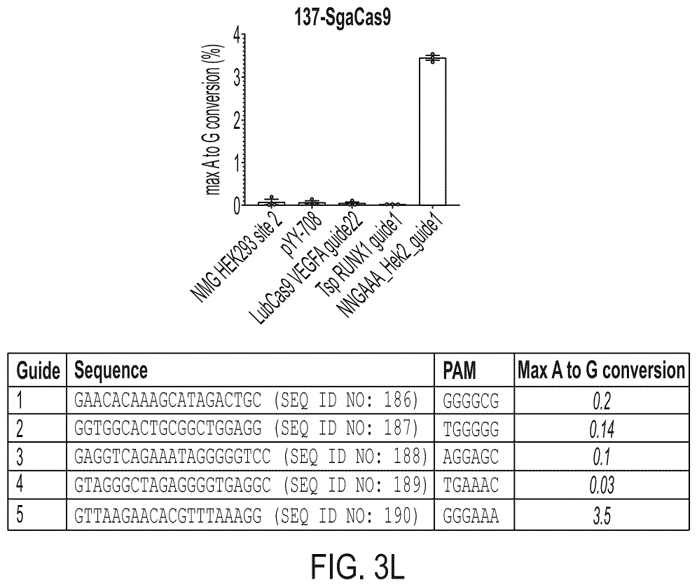

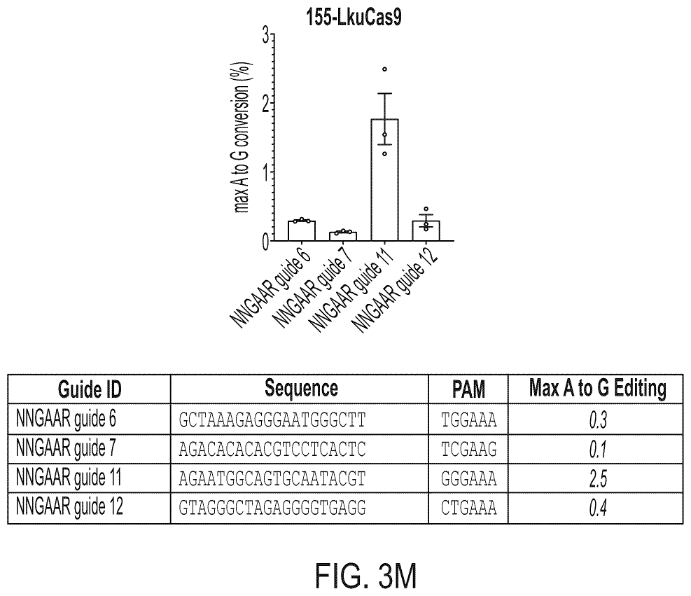

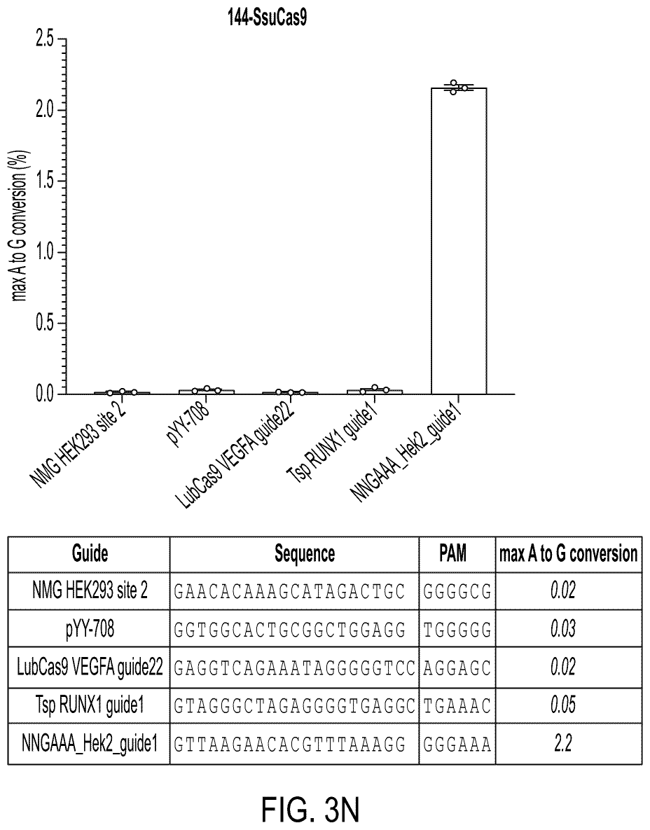

Table 8 shows exemplary guide RNA sequences used with Seq2Cas9. FIG. 3A is a graph that shows results of adenine to guanine base (A-to-G) conversion percentage achieved with a base editor comprising an ABE fused to the N-terminus of a Seq2Cas9 D10A mutant.

TABLE 8
Guide RNA Sequences and PAM
Sequences used with Seq2Cas9

| | | | Max |
|---|---|---|---|
| Guide | | | A to G |
| No. | Sequence | PAM | editing |
| 1 | GGTCGTCAATGAAAGGAGAT | AAGGTC | 0.103 |
| | (SEQ ID NO: 119) | | |
| 2 | GCTAGGAATATTGAAGGGGG | CAGGGG | 68.188 |
| | (SEQ ID NO: 120) | | |
| 3 | GCTCCCATCACATCAACCGG | TGGCGC | 10.103 |
| | (SEQ ID NO: 121) | | |
| 4 | GGGCAACCACAAACCCACGA | GGGCAG | 24.233 |
| | (SEQ ID NO: 122) | | |
| 5 | GAGTCGACGAGTTGAAGATG | AAGCCC | 0.333 |
| | (SEQ ID NO: 123) | | |

Table 9 shows exemplary guide RNA sequences used with EhiCas9. FIG. 3B is a graph that shows results of indel frequency and adenine to guanine base (A-to-G) conversion percentage achieved with a base editor comprising an ABE fused to the N-terminus of a EhiCas9 D10A mutant.

TABLE 9
Guide RNA Sequences and PAM Sequences
used with EhiCas9

| | | | Max |
|---|---|---|---|
| Guide | | | A to G |
| No. | Sequence | PAM | editing |
| 1 | GTCTGTTACTCGCCTGTCAA | GTGGCG | 1.206 |
| | (SEQ ID NO: 124) | | |
| 2 | ATAAAAGGAAAAGTCACTCT | GGGGAA | 2.845 |
| | (SEQ ID NO: 125) | | |
| 3 | GTGCCAGAAACAGGGGTGAC | GGGAGG | 9.567 |
| | (SEQ ID NO: 126) | | |
| 4 | GTGGGCAACCACAAACCCAC | GAGGGC | 0.387 |
| | (SEQ ID NO: 127) | | |
| 5 | GCAGAGCAAATACCAGAGAT | AAGAGA | 16.694 |
| | (SEQ ID NO: 128) | | |
| 6 | GGTGCTCAATGAAAGGAGAT | AAGGTC | 5.565 |
| | (SEQ ID NO: 129) | | |
| 7 | CGAGCAGCGTCTTCGAGAGT | GAGGAC | 29.696 |
| | (SEQ ID NO: 130) | | |
| 8 | GCTCCCATCACATCAACCGG | TGGCGC | 29.022 |
| | (SEQ ID NO: 131) | | |
| 9 | GGGCAACCACAAACCCACGA | GGGCAG | 43.877 |
| | (SEQ ID NO: 132) | | |
| 10 | GAGTCGACGAGTTGAAGATG | AAGCCC | 0.642 |
| | (SEQ ID NO: 133) | | |
| 11 | TGGCCATCAAGGATGCCCAC | GAGAAA | 0.375 |
| | (SEQ ID NO: 134) | | |
| 12 | CCCCCACCAAGGTTCACAGC | CTGAAA | 0.225 |
| | (SEQ ID NO: 135) | | |
| 13 | AGAATGGCAGTGCAATACGT | GGGAAA | 31.428 |
| | (SEQ ID NO: 136) | | |
| 12 | GTAGGGCTAGAGGGGTGAGG | CTGAAA | 0.048 |
| | (SEQ ID NO: 137) | | |

Table 10 shows exemplary guide RNA sequences used with SeqCas9. FIG. 3C is a graph that shows results of adenine to guanine base (A-to-G) conversion percentage achieved with a base editor comprising an ABE fused to the N-terminus of a SeqCas9 D10A mutant.

TABLE 10
Guide RNA Sequences and PAM Sequences
for use with SeqCas9

| | | | Max |
|---|---|---|---|
| Guide | | | A to G |
| No. | Sequence | PAM | conversion |
| 1 | GAACACAAAGCATAGACTGC | GGGGCG | 0.89 |
| | (SEQ ID NO: 138) | | |
| 2 | GGTGGCACTGCGGCTGGAGG | TGGGGG | 1.37 |
| | (SEQ ID NO: 139) | | |
| 3 | GAGGTCAGAAATAGGGGGTCC | AGGAGC | 0.4 |
| | (SEQ ID NO: 140) | | |
| 4 | GTAGGGCTAGAGGGGTGAGGC | TGAAAC | 0.05 |
| | (SEQ ID NO: 141) | | |
| 5 | GTTAAGAACACGTTTTAAAGG | GGGAAA | 30.2 |
| | (SEQ ID NO: 142) | | |

Table 11 shows exemplary guide RNA sequences used with SsiCas9. FIG. 3D is a graph that shows results of adenine to guanine base (A-to-G) conversion percentage achieved with a base editor comprising an ABE fused to the N-terminus of a SsiCas9 D10A mutant.

TABLE 11
Guide RNA Sequences and PAM Sequences
for use with SSiCas9

| | | | Max |
|---|---|---|---|
| Guide | | | A to G |
| No. | Sequence | PAM | conversion |
| 1 | GAACACAAAGCATAGACTGC | GGGGCG | 9.4 |
| | (SEQ ID NO: 143) | | |
| 2 | GGTGGCACTGCGGCTGGAGG | TGGGGG | 12.9 |
| | (SEQ ID NO: 144) | | |
| 3 | GAGGTCAGAAATAGGGGGTCC | AGGAGC | 4.6 |
| | (SEQ ID NO: 145) | | |
| 4 | GTAGGGCTAGAGGGGTGAGGC | TGAAAC | 0.06 |
| | (SEQ ID NO: 146) | | |
| 5 | GTTAAGAACACGTTTAAAGG | GGGAAA | 14.6 |
| | (SEQ ID NO: 147) | | |

Table 12 shows exemplary guide RNA sequences used with SinCas9. FIG. 3E is a graph that shows results of adenine to guanine base (A-to-G) conversion percentage achieved with a base editor comprising an ABE fused to the N-terminus of a SinCas9 D9A mutant.

TABLE 12
Guide RNA Sequences and PAM Sequences
for use with SinCas9

| | | | Max |
|---|---|---|---|
| Guide | | | A to G |
| No. | Sequence | PAM | conversion |
| 1 | GAACACAAAGCATAGACTGC | GGGGCG | 0.02 |
| | (SEQ ID NO: 148) | | |
| 2 | GGTGGCACTGCGGCTGGAGG | TGGGGG | 0.08 |
| | (SEQ ID NO: 149) | | |
| 3 | GAGGTCAGAAATAGGGGGTCC | AGGAGC | 0.02 |
| | (SEQ ID NO: 150) | | |
| 4 | GTAGGGCTAGAGGGGTGAGGC | TGAAAC | 0.1 |
| | (SEQ ID NO: 151) | | |
| 5 | GTTAAGAACACGTTTAAAGG | GGGAAA | 13.1 |
| | (SEQ ID NO: 152) | | |

Table 13 shows exemplary guide RNA sequences used with SsaCas9. FIG. 3F is a graph that shows results of adenine to guanine base (A-to-G) conversion percentage achieved with a base editor comprising an ABE fused to the N-terminus of a SsaCas9 D11A mutant.

TABLE 13
Guide RNA Sequences and PAM Sequences
for use with SsaCas9

| | | | Max |
|---|---|---|---|
| | | | A to G |
| Guide | Sequence | PAM | editing |
| 1 | GTCTGTTACTCGCCTGTCAA | GTGGCG | 0.075 |
| | (SEQ ID NO: 153) | | |
| 2 | GTGGGCAACCACAAACCCAC | GAGGGC | 0.437 |
| | (SEQ ID NO: 154) | | |
| 3 | GCAGAGCAAATACCAGAGAT | AAGAGA | 7.681 |
| | (SEQ ID NO: 155) | | |
| 4 | GGTGCTCAATGAAAGGAGAT | AAGGTC | 0.05 |
| | (SEQ ID NO: 156) | | |
| 5 | CGAGCAGCGTCTTCGAGAGT | GAGGAC | 0.159 |
| | (SEQ ID NO: 157) | | |
| 6 | GCTCCCATCACATCAACCGG | TGGCGC | 12.378 |
| | (SEQ ID NO: 158) | | |
| 7 | GGGCAACCACAAACCCACGA | GGGCAG | 11.22 |
| | (SEQ ID NO: 159) | | |
| 8 | GAGTCGACGAGTTGAAGATG | AAGCCC | 0.048 |
| | (SEQ ID NO: 160) | | |

Table 14 shows exemplary guide RNA sequences used with Ssc2Cas9. FIG. 3G is a graph that shows results of indel frequency and adenine to guanine base (A-to-G) conversion percentage achieved with a base editor comprising an ABE fused to the N-terminus of a Ssc2Cas9 D9A mutant.

TABLE 14
Guide RNA Sequences and PAM Sequences
for use with Ssc2Cas9

| | | | Max |
|---|---|---|---|
| Guide | | | A to G |
| No. | Sequence | PAM | conversion |
| 1 | GAACACAAAGCATAGACTGC | GGGGCG | 8.5 |
| | (SEQ ID NO: 161) | | |
| 2 | GGTGGCACTGCGGCTGGAGG | TGGGGG | 11.6 |
| | (SEQ ID NO: 162) | | |
| 3 | GAGGTCAGAAATAGGGGGTCC | AGGAGC | 1.4 |
| | (SEQ ID NO: 163) | | |
| 4 | GTAGGGCTAGAGGGGTGAGGC | TGAAAC | 0.03 |
| | (SEQ ID NO: 164) | | |
| 5 | GTTAAGAACACGTTTTAAAGG | GGGAAA | 4.9 |
| | (SEQ ID NO: 165) | | |

Table 15 shows exemplary guide RNA sequences used with Sor2Cas9. FIG. 3H is a graph that shows results of indel frequency and adenine to guanine base (A-to-G) conversion percentage achieved with a base editor comprising an ABE fused to the N-terminus of a Sor2Cas9 D9A mutant.

TABLE 15
Guide RNA Sequences and PAM Sequences
for use with Sor2Cas9

| | | | Max |
|---|---|---|---|
| Guide | | | A to G |
| No. | Sequence | PAM | conversion |
| 1 | GAACACAAAGCATAGACTGC | GGGGCG | 0.02 |
| | (SEQ ID NO: 166) | | |
| 2 | GGTGGCACTGCGGCTGGAGG | TGGGGG | 0.07 |
| | (SEQ ID NO: 167) | | |
| 3 | GAGGTCAGAAATAGGGGGTCC | AGGAGC | 1 |
| | (SEQ ID NO: 168) | | |
| 4 | GTAGGGCTAGAGGGGTGAGGC | TGAAAC | 8.7 |
| | (SEQ ID NO: 169) | | |
| 5 | GTTAAGAACACGTTTAAAGG | GGGAAA | 0.1 |
| | (SEQ ID NO: 170) | | |

Table 16 shows exemplary guide RNA sequences used with SorCas9. FIG. 3I is a graph that shows results of adenine to guanine base (A-to-G) conversion percentage achieved with a base editor comprising an ABE fused to the N-terminus of a SorCas9 D9A mutant.

TABLE 16
Guide RNA Sequences and PAM Sequences
for use with SorCas9

| | | | Max |
|---|---|---|---|
| Guide | | | A to G |
| No. | Sequence | PAM | conversion |
| 1 | GAACACAAAGCATAGACTGC | GGGGCG | 0.35 |
| | (SEQ ID NO: 171) | | |
| 2 | GGTGGCACTGCGGCTGGAGG | TGGGGG | 6.7 |
| | (SEQ ID NO: 172) | | |
| 3 | GAGGTCAGAAATAGGGGGTCC | AGGAGC | 0.02 |
| | (SEQ ID NO: 173) | | |
| 4 | GTAGGGCTAGAGGGGTGAGGC | TGAAAC | 0.35 |
| | (SEQ ID NO: 174) | | |
| 5 | GTTAAGAACACGTTTAAAGG | GGGAAA | 0.4 |
| | (SEQ ID NO: 175) | | |

Table 17 shows exemplary guide RNA sequences used with SwaCas9. FIG. 3J is a graph that shows results of adenine to guanine base (A-to-G) conversion percentage achieved with a base editor comprising an ABE fused to the N-terminus of a SwaCas9 mutant.

TABLE 17
Guide RNA Sequences and PAM Sequences
for use with SwaCas9

| | | | Max |
|---|---|---|---|
| | | | A to G |
| Guide | Sequence | PAM | conversion |
| 1 | GAACACAAAGCATAGACTGC | GGGGCG | 0.86 |
| | (SEQ ID NO: 176) | | |
| 2 | GGTGGCACTGCGGCTGGAGG | TGGGGG | 6.5 |
| | (SEQ ID NO: 177) | | |
| 3 | GAGGTCAGAAATAGGGGGTCC | AGGAGC | 0.08 |
| | (SEQ ID NO: 178) | | |
| 4 | GTAGGGCTAGAGGGGTGAGGC | TGAAAC | 0.04 |
| | (SEQ ID NO: 179) | | |
| 5 | GTTAAGAACACGTTTAAAGG | GGGAAA | 0.02 |
| | (SEQ ID NO: 180) | | |

Table 18 shows exemplary guide RNA sequences used with SscCas9. FIG. 3K is a graph that shows results of adenine to guanine base (A-to-G) conversion percentage achieved with a base editor comprising an ABE fused to the N-terminus of a SscCas9 mutant.

TABLE 18
Guide RNA Sequences and PAM Sequences
for use with SscCas9

| | | | Max |
|---|---|---|---|
| | | | A to G |
| Guide | Sequence | PAM | conversion |
| 1 | GAACACAAAGCATAGACTGC | GGGGCG | 0.05 |
| | (SEQ ID NO: 181) | | |
| 2 | GGTGGCACTGCGGCTGGAGG | TGGGGG | 3.7 |
| | (SEQ ID NO: 182) | | |
| 3 | GAGGTCAGAAATAGGGGGTCC | AGGAGC | 1.4 |
| | (SEQ ID NO: 183) | | |
| 4 | GTAGGGCTAGAGGGGTGAGGC | TGAAAC | 0.02 |
| | (SEQ ID NO: 184) | | |
| 5 | GTTAAGAACACGTTTAAAGG | GGGAAA | 0.4 |
| | (SEQ ID NO: 185) | | |

Table 19 shows exemplary guide RNA sequences used with SgaCas9. FIG. 3L is a graph that shows results of adenine to guanine base (A-to-G) conversion percentage achieved with a base editor comprising an ABE fused to the N-terminus of a SgaCas9 mutant.

TABLE 19
Guide RNA Sequences and PAM Sequences
for use with SgaCas9

| | | | Max |
|---|---|---|---|
| | | | A to G |
| Guide | Sequence | PAM | conversion |
| 1 | GAACACAAAGCATAGACTGC | GGGGCG | 0.2 |
| | (SEQ ID NO: 186) | | |
| 2 | GGTGGCACTGCGGCTGGAGG | TGGGGG | 0.14 |
| | (SEQ ID NO: 187) | | |
| 3 | GAGGTCAGAAATAGGGGGTCC | AGGAGC | 0.1 |
| | (SEQ ID NO: 188) | | |
| 4 | GTAGGGCTAGAGGGGTGAGGC | TGAAAC | 0.03 |
| | (SEQ ID NO: 189) | | |
| 5 | GTTAAGAACACGTTTAAAGG | GGGAAA | 3.5 |
| | (SEQ ID NO: 190) | | |

Table 20 shows exemplary guide RNA sequences used with LkuCas9. FIG. 3M is a graph that shows results of adenine to guanine base (A-to-G) conversion percentage achieved with a base editor comprising an ABE fused to the N-terminus of a LkuCas9 mutant.

TABLE 20
Guide RNA Sequences and PAM Sequences
for use with LkuCas9

| | | | Max |
|---|---|---|---|
| | | | A to G |
| Guide | Sequence | PAM | Editing |
| 1 | GCTAAAGAGGGAATGGGCTT | TGGAAA | 0.3 |
| 2 | AGACACACACGTCCTCACTC | TCGAAG | 0.1 |
| 3 | AGAATGGCAGTGCAATACGT | GGGAAA | 2.5 |
| 4 | GTAGGGCTAGAGGGGTGAGG | CTGAAA | 0.4 |

Table 21 shows exemplary guide RNA sequences used with SsuCas9. FIG. 3N is a graph that shows results of adenine to guanine base (A-to-G) conversion percentage achieved with a base editor comprising an ABE fused to the N-terminus of a SsuCas9 mutant.

TABLE 21
Guide RNA Sequences and PAM Sequences
for use with SsuCas9

| | | | Max |
|---|---|---|---|
| | | | A to G |
| Guide | Sequence | PAM | conversion |
| 1 | GAACACAAAGCATAGACTGC | GGGGCG | 0.02 |
| | (SEQ ID NO: 191) | | |
| 2 | GGTGGCACTGCGGCTGGAGG | TGGGGG | 0.03 |
| | (SEQ ID NO: 192) | | |
| 3 | GAGGTCAGAAATAGGGGGTCC | AGGAGC | 0.02 |
| | (SEQ ID NO: 193) | | |
| 4 | GTAGGGCTAGAGGGGTGAGGC | TGAAAC | 0.05 |
| | (SEQ ID NO: 194) | | |
| 5 | GTTAAGAACACGTTTAAAGG | GGGAAA | 2.2 |
| | (SEQ ID NO: 195) | | |

TABLE 22
Summary of Cas9 Orthologs, Predicted PAM,
Seed Sequence and A to G Editing Efficiency

| | | | | % | % |
|---|---|---|---|---|---|
| | | Max | | identity | identity |
| | Predicted | A to G | | to | to |
| Nuclease | PAM | Editing | Seed | SpyCas9 | SauCas9 |
| Seq2Cas9 | NRGNR | 68% | ScoCas9 | 57.3 | 21.8 |
| EhiCas9 | NRG | 44% | ScoCas9 | 48.6 | 22.4 |
| SeqCas9 | NNGR | 30% | LubCas9 | 21.7 | 36.5 |
| SsiCas9 | NNGRRT | 14.60% | LubCas9 | 20.8 | 86.3 |
| SinCas9 | NNAAAA | 13% | LubCas9 | 20.4 | 36.4 |
| SsaCas9 | NGGNG | 12.40% | ScoCas9 | 58.5 | 20.3 |
| Ssc2Cas9 | NNGNRG | 11.60% | LubCas9 | 21.1 | 35 |
| Sor2Cas9 | NNAAAC | 8.70% | LubCas9 | 21.5 | 35.7 |
| SorCas9 | NNRAAG | 6.70% | LubCas9 | 21.6 | 35.5 |
| SwaCas9 | NNGR | 6.50% | LubCas9 | 22.1 | 64.2 |
| SscCas9 | NNGR | 3.70% | LubCas9 | 21 | 96.2 |
| SgaCas9 | NNAYAA | 3.50% | LubCas9 | 20.3 | 36.3 |
| LkuCas9 | NNGAAA | 2.50% | LubCas9 | 31.2 | 20.1 |
| SsuCas9 | NNAAA | 2.00% | LubCas9 | 21.1 | 36.4 |

Table 23 discloses sequences for exemplary Cas9 adenosine or adenine for base editing functions.

TABLE 23
Sequences of exemplary Cas9 adenosine or
adenine base editors

| | |
|---|---|
| Sequence ID No. | |
| (description) | Components of DNA cleavage assay |

Amino Acid Sequence of Adenine Deaminase, TadA8.13m-
nickase fused to the N-terminal of nickase Seq2Cas9
(ABE-nSeq2Cas9, D10A mutant)
MPAAKRVKLDGSEVEFSHEYWMRHALTLAKRARDEREVPVGAVLVLNNRVIGEGWNRAIG
LHDPTAHAEIMALRQGGLVMQNYRLYDATLYVTFEPCVMCAGAMIHSRIGRVVFGVRNAK
TGAAGSLMDVLHHPGMNHRVEITEGILADECAALLCRFFRMPRRVFNAQKKAQSSTDGSS
GSETPGTSESATPESSGPKKKRKVGTMEKSYSIGLAIGTNSVGWSVITDDYKVPAKKMRV
LGNTDKKYIKKNLLGALLFDSGETAEATRLKRTARRRYTRRKNRLRYLQEIFAKEMAKVD
ESFFQRLDESFLTDDDKTFDSHPIFGNKAEEDAYHQKFPTIYHLRKHLADSQEKADLRLI
YLALAHMIKFRGHFLYDNFNDDNFDWRNIDIQKRYEEFIETYDSTLGESYLADISVDAAS
ILEEKVSKTERLENLLKYYPTEKKTTFFGNLIKLILGQQAKFKAIFNLEDEISLQFATPT
YDEDLEELLGKIDNGDSYSELFVAAQNLYNTILLASFLKTDNKSAKAPLSTSMIERYENH
KKDLAKLKDFVKKNCPDQYHDIFRDKSKNGYAGYIDNGVSQNDFYTFIGKCLEESLKKDK
GAQYFLDKIDRDDFLRKQRTFDNGAFPYQIHLQEMHAILRRQGDYYPFLKENQDKIEKIL
TFRIPYYVGPLARKDSRFAWANYRSDEAITPWNFDEVVDKEKSAEKFITRMTLNDLYLPE
EKVLPKHSLIYETFTVYNELTNIKYVNDQGNAIHFDSELKEKIFNQLFKENRKVSKKTLI
DFLNNSEGIYTDKLVGIDEEVKYLNASLGTYHDLKKILESFMDDEINEKIIEDIIQTLTL
FEDIEMKRQRLQKYDDIFTPKQLKELARRNYTGWGRLSYKLINGIRNKENNKTILDYLKN
GNRNFMQLINDDRLSFKQIIIDARKIEKLDNIESVVYNLPGSPAIKKGILQSIKIVDELV
KVMGHNPDNIVIEMARENQTTNQGKNRSQQRLKRLQDSMSNFKDSSISLKDVDNSDLQND
RLFLYYIQNGKDMYTGEELDIDHLSDYDIDHIIPQSFIKDNSIDNRVLTSSAKNRGKSDD
VPGRDVVLKMKPFWKKLYDVKLISKRKFDNLTKSEHGGLTESDKAGFIKRQLVETRQITK
YVAQILDGRFNTKRDDNNKVIRDVKVITLKSSLVSQFRKDFGFYKVREINDYHHAHDAYL
NAVVGTAILKKYPKLAPEFVYGEYKKCDVRKLIAKSGDKSEIGKATAKYFFYSNLMNFFK
RVIRYSNGMIVVRPVIEYSKDTGEIAWDKEKDFKTVCKVLSCPQVNIVKKVEKQSHGLDR
GKPKGFYNANPSPKPKKGSKVNLVPIKANLNPKNYGGYAGISNSYAVLVDATIEKGAKKK
LTRIQEFQGISIIDREKYEKNKVEFLKGLGYKEIYSIITLPKYSLFELADGSRRMLASIL
STNNKRGEIHKGNELVLPAKYIPLLYHANRIHNTFETGHREYVEKHIAEFKEIAEIILEF
NNKYVNAKKNSSIIEKALESFDSFSLDEICDSFVGKLKKNNTKKNSGLFELVSLGSASDF
EFLETKVPRYRDYTPSSLLNATLIHQSITGLYETRIDLSKLGEEKRPAATKKAGQAKKKK
GSYPYDVPDYAYPYDVPDYAYPYDVPDYA (SEQ ID NO: 29).
Amino Acid Sequence of Adenine Deaminase, TadA8.13m-
nickase fused to the N-terminal of nickase EhiCas9
(ABE-nEhiCas9, D10A mutant)
MPAAKRVKLDGSEVEFSHEYWMRHALTLAKRARDEREVPVGAVLVLNNRVIGEGWNRAIG
LHDPTAHAEIMALRQGGLVMQNYRLYDATLYVTFEPCVMCAGAMIHSRIGRVVFGVRNAK
TGAAGSLMDVLHHPGMNHRVEITEGILADECAALLCRFFRMPRRVFNAQKKAQSSTDGSS
GSETPGTSESATPESSGPKKKRKVGTMTKDYTIGLAIGTNSVGWAVLTDDYQLMKRKMSV
HGNTEKKKIKKNFWGARLFDEGQTAEFRRTKRTNRRRLARRKYRLSKLQDLFAEELCKQD
DCFFVRLEESFLVPEEKQYKPASIFPTLEEEKEYYQKYPTIYHLRQKLVDSTEKEDLRLV
YLALAHLLKYRGHELFEGDLDTENTSIEESFRVFLEQYSKQSDQPLIVHQPVLTILTDKL
SKTKKVEEILKYYPTEKINSFFAQCLKLIVGNQANFKRIFDLEAEVKLQFSKETYEEDLE
SLLEKIGDEYLDIFLQAKKVHDAILLSEIISSTVKHTKAKLSSGMVERYERHKADLAKFK
QFVKENVPQKSTAFFKDTTKNGYAGYIKGKTTQEEFYKFVKKELSGVVGSEPFLEKIDQE
TFLLKQRTYTNGVIPHQVHLIELKAIIDQQKQHYPFLEEAGPKIIALFKFRIPYYVGPLA
KEQEASSFAWIERKTAEKIHPWNFSEVVDIEKSAMRFIQRMTKQDTYLPTEKVLPKNSLL
YQKYMIFNELTKVSYKDERGVKQYFSGDEKQQIFKQLFQKERGKITVKKLQNFLYTHYHI
ENAQIFGIEKAFNASYSTYHDFMKLAKTNQKAMQEWLEQPEMEPIFEDIVKILTIFEDRQ
MIKHQLSKYQEVFGEKLLKEFARKHYTGWGRFSAKLIHGIRDRKTNKTILDYLINDDDVP
ANRNRNLMQLINDEHLSFKEEIAKATVFSKHKSLVDVIQDLPGSPAIKKGIWQSLKIVEE
LIAIIGYKPKNIVIEMARENQKTHRTKPRLKALENGLKQIGSTLLKEQPTDNKALQKERL
YLYYLQNGRDMYTGEPLEIENLHQYEVDHIIPRSFIVDNSIDNKVLVARKQNQKKRDDVP
KKQIVNEQRIFWNQLKEAKLISPKKYAYLTKIELTPEDKARFIQRQLVETRQITKHVANI
LHQSFHQEEEGTDCDGVQIITLKATLTSQFRQTFGLYKVREINPHHHAHDAYLNGFIANV
LLKRYPKLAPEFVYGKYVKYSLARENKATAKKEFYSNILKFFESDEPFCDENGEIYWEKS
HHLPRIKKVLSSHQVNVVKKVEQQKGGFYKETVNSKEKPDKLIERKNNWDVTKYGGFGSP
VIAYAIAFVYAKGKTQKKTRAIEGITIMEQAAFEKDPTTFLKEKGFPQVTEFIKLPKYTL
FEFDNGRRRFLASHKESQKGNPFILSDQLVTLLYHAQHYDKITYQESFDYVNTHLSDFSA
ILTEVLAFAEKYTLADKNIERIQELYEENKYGEISMIAQSFLQLLQFNAIGAPADFKFFG
VTIPRKRYTSLTEIWDATIIYQSVTGLYETRIRMGDLWAGEQKRPAATKKAGQAKKKKGS
YPYDVPDYAYPYDVPDYAYPYDVPDYA (SEQ ID NO: 30)
Amino Acid Sequence of Adenine Deaminase, TadA8.13m
fused to the N-terminal of nickase SeqCas9 (ABE-
nSeqCas9, D10A mutant).
MPAAKRVKLDGSEVEFSHEYWMRHALTLAKRARDEREVPVGAVLVLNNRVIGEGWNRAIG
LHDPTAHAEIMALRQGGLVMQNYRLYDATLYVTFEPCVMCAGAMIHSRIGRVVFGVRNAK
TGAAGSLMDVLHHPGMNHRVEITEGILADECAALLCRFFRMPRRVFNAQKKAQSSTDGSS
GSETPGTSESATPESSGPKKKRKVGTMTNGKILGLAIGVASVGVGIIEAKTGKVIHANSR
LFSAANAENNAERRGFRGARRLTRRKKHRVKRVRDLFEKYDISTDFRNLNLNPYELRVKG
LSEQLTNEELFAALRTIAKRRGISYLDDAEDDSTGSSDYAKSIDENRRLLKSMTPGQIQL
ERLEKYGQLRGNFTVYDENGEAHRLINVESTSDYKNEARKILETQSNYNKQITDEFIEDY
IEILTQKRKYYHGPGNEKSRTDYGRFRTDGTTLENIFGILIGKCSFYPEEYRASKASYTA
QEFNFLNDLNNLKVPTETGKLSTEQKEYLVDFAKNSKTLGASKLLKEIAKLVDGDVKDIS
GYREDSKGKPDLHTFEPYRKLKFNLTTVDIDNLSRDILDKLANILTLNTEREGIEDAINR
NLPEQFTKEQISEIVQIRKSQSSAFNKGWHSFSAKLMNELIPELYATSEEQMTILTRLEK
FKATKKSSKNTKTIDEKEITDEIYNPVVAKSVRQTIKIINAAVKKYGDFDKIVIEMPRDK
NADDEKKFIDKKNKENKKEKDDSLKRAAYLYNGTDKLPDDVFHGNKQLATKIRLWYQQGE
RCLYSGKPILIQDLVHNSNNFEIDHILPLSLSFDDSLANKVLVYAWTNQEKGQKTPYQVI
DSMDAAWSFREMKDYVLKQKGIGKKKREYLLTTENIDKIEVKKKFIERNLVDTRYASRVV
LNSLQTALKELGKDTKVSVVRGQFTSQLRRKWKIDKSRETYHHHAVDALIIAASSQLKLW
QKHENLMFENYGENQVVNKETGEILSISDDEYKELVFQPPYQGFVNTISSKAFEDEILFS
YQVDSKFNRKVSDATIYSTRKAKLGKDKKEETYVLGKIKDIYSQDGFDTFIKRYKKDKTQ
FLMYQKDPLTWENVIEVILRDYPTTKKSEDGKNDVKCNPFEEYRRENGLICKYSKKGKGT
PIKSLKYYDKKLGNHISITPKESKNDVVLQSLNPWRADLYFNPDTLKYELMGLKYSDLSF
EKGTGKYHISQEKYDEIKEKEGIGQNSEFKFTLYRNDLILIKDTESGEQEIYRFLSRTMP
NVKHYVELKPYDKEKFNGGQELIKSLGEADKVGRCLKGLSKPGISIYKVRTDVLGNKFFV
KKEGDKPKLDFKNNKKKRPAATKKAGQAKKKKGSYPYDVPDYAYPYDVPDYAYPYDVPDY
A (SEQ ID NO: 31).
Amino Acid Sequence of Adenine Deaminase, TadA8.13m
fused to the N-terminal of nickase SsiCas9 (ABE-
nSsiCas9, D10A mutant).
MPAAKRVKLDGSEVEFSHEYWMRHALTLAKRARDEREVPVGAVLVLNNRVIGEGWNRAIG
LHDPTAHAEIMALRQGGLVMQNYRLYDATLYVTFEPCVMCAGAMIHSRIGRVVFGVRNAK
TGAAGSLMDVLHHPGMNHRVEITEGILADECAALLCRFFRMPRRVFNAQKKAQSSTDGSS
GSETPGTSESATPESSGPKKKRKVGTMNGLVLGLAIGIASVGVGILNKETGEIIHVNSRI
FPAATADSNVERRGFRQGRRLGRRKKHRSARLNDLFEEFGFITDFSAVPLNLNPYALRVK
GLSEELTNEELFIALKNIIKRRGISYLDDASEDGETASNEYGKAVEENRKLLADKTPGQI
QLERFEKYGQVRGDFTVVENGENHRLINVESTSAYKKEAERILRRQQEFNVRISDEFIEA
YLTILTGKRKYYHGPGNEKSRTDYGRFRTDGTTLDNIFGILIGKCTFYPDEYRAAKASYT
AQEFNLLNDLNNLTVPTETKKLRPEQKRQIVEYARTAKTLGTPTLLKYIAKLVDGSIDDI
KGYRIDKSDKPEMHTFDAYRKMRTLELVDVDILSRETLDDLAYILTLNTESEGILEALNS
KMPGTFTKEQIDELIQFRKKNSAVFGKGWHNFSLKLMNELISELYETSEEQMTILTRLGK
QRSREISKRTKYIDEKELTDEIYNPVVAKSVRQAIKIINLATKKYGIFDNIVIEMARESN
EDDEKKAIQNVQKANEDEKKAAMEKAADLYNGKKELPDSIFHGHKELATKIRLWHQQGER
CLYTGKNISIHDLIHNPHQYEIDHILPLSLSFDDGLANKVLVLATANQEKGQRTPFQALD
SMDDAWSYIEFKQYVRNSKSLSNKKKDYLLTEEDISKIEVKQKFIERNLVDTRYSSRVVL
NTLQEFYKTNDFDTKISVVRGQFTSQLRRKWKIEKSRDTYHHHAVDALIIAASSQLRLWK
KQNNPLISYREGQLVDPETGEILSLTDDEYKELVFRPPYDYFVDTLKSKSFEDSILFSYQ
VDSKYNRKISDATIYGTRKAQLGKDKQEETYVLGKIKDIYSQKGYEDFIKRYKKDTTQFL
MYHKDPQTFAKVIEEILKTYPDKELNEKGKEIPCNPFEKYRQENGPIRKYSKKGKGPEIK
SLKYYDNKLGNHIDITPVNSQNQVVLQSLKPWRTDVYFNPQTSKYELMGLKYSDLRFEKG
SGSYGISPEKYNKVKAKEGVDEDSEFKFTLYKNDLILIKDTETGEQQLFRYGSRNDTSKH
YVELKPYEKAKFEGNQQLMNLLGTVAKGGQCLKGINKPNLSIYKVKTDVLGNKHFIKKEG
DQPQLNFKKKIKRPAATKKAGQAKKKKGSYPYDVPDYAYPYDVPDYAYPYDVPDYA
(SEQ ID NO: 32).
Amino Acid Sequence of Adenine Deaminase, TadA8.13m
fused to the N-terminal of nickase Sin Cas9
(ABE-nSinCas9, D9A mutant).
MPAAKRVKLDGSEVEFSHEYWMRHALTLAKRARDEREVPVGAVLVLNNRVIGEGWNRAIG
LHDPTAHAEIMALRQGGLVMQNYRLYDATLYVTFEPCVMCAGAMIHSRIGRVVFGVRNAK
TGAAGSLMDVLHHPGMNHRVEITEGILADECAALLCRFFRMPRRVFNAQKKAQSSTDGSS
GSETPGTSESATPESSGPKKKRKVGTMNNSYILGLAIGITSVGYGIIEYETRDVIDAGVR
LFKEANVENNEGRRSKRGARRLKRRRRHRLQRVKKMLFDYKLLNEDSEISGINPYEARVK
GLSEKLSDEEFSAALLHLAKRRGVHNVSDVEEDTGNELSTKEQIARNNKALEDKYVAELQ
LERLKEQGEVRGAANRFKTSDYIKEAKQLLKTQSDYHKIDETFIETYISLLETRRTYYEG
PGEGSPFGWKDIKEWYEMLMGHCTYFPEELRSVKYAYNADLYNALNDLNNLVIARDENEK
LEYYEKFQIIENVFKQKKKPTLKQIAKEILVNEEDIKGYRVTSTGKPEFTNFKIYHDIKG
ITSRKEILENADLLNQIAEILTIYQSAEDIQEELAELEPKLTQEEIEQISNLTGYTGTHR
LSLKAINLILDELWNTSDNQMTIFNRLKLVPKKVDLSQQKEIPTSLVDDFILSPVVKRSF
IQSIKVINAIIKKFGLPKDIIIELAREKNSKEAQKFINEMQKRNRQTNERIEKIIKETGK
EKAKFLIEKIKLHDMQEGKCLYSLESIPLEDLLNNPYHYEVDHIIPRSVSFDNSFNNKVL
VKQEENSKKGNRTPFQYLSSSDAKISYETFKKHILNLSKGKGRVSKKKKEYLLEERDINR
FSVQKDFINRNLVDTRYATRELMNLLRSYFRVNDLDVKVKSINGGFTSFLRTKWKFKKER
NQGYKHHAEDALVIANADFIFKEWKKLDTTNKVMENQTVEEQQAENMPGIETDDEYKEIF
VIPRQIQSIKDFKDYKYSHRVDKKPNRELVNDTLYSTRKDDKGNTLIINNIKGLYDKDND
KLKNLIKKSPEKLLMYHHDPQTYQKLKTIMEQYSNEKNPLYKYHEETGNYLTKYSKKDNG
PIIKKVKYYGKKLNAHLDITNDYSNSQNKIVKLSLKPYRFDVYLDNGGYKFVTVKNLDVI
KKEGFFKIDSNAYEKAKSEKKIDENAVFIASFYNNDLIKIDGELYRIVGVNNDTRNVVEL
NMIPITYKEYLENINDKRTPRILKTISQKTYSIEKYSTDILGNLYKVKSKKKPQMIMKGK
RPAATKKAGQAKKKKGSYPYDVPDYAYPYDVPDYAYPYDVPDYA (SEQ ID NO:
33).
Amino Acid Sequence of Adenine Deaminase, TadA8.13m
fused to the N-terminal of nickase SsaCas9
(ABE-nSsaCas9, D11A mutant)
MPAAKRVKLDGSEVEFSHEYWMRHALTLAKRARDEREVPVGAVLVLNNRVIGEGWNRAIG
LHDPTAHAEIMALRQGGLVMQNYRLYDATLYVTFEPCVMCAGAMIHSRIGRVVFGVRNAK
TGAAGSLMDVLHHPGMNHRVEITEGILADECAALLCRFFRMPRRVFNAQKKAQSSTDGSS
GSETPGTSESATPESSGPKKKRKVGTMENKNYSIGLAIGTNSVGWAVITDDYKVPSKKMK
VFGNTDKHFIKKNLIGALLFDEGATAEDRRLKRTARRRYTRRKNRLRYLQEIFSEEISKL
DSSFFHRLDDSFLVPKDKRGSKYPIFATLEEEKEYHKKFPTIYHLRKHLADSKEKTDLRL
IYLALAHMIKYRGHFLYEESFDIKNNDIQKIFNEFISIYDNTFEGSSLSGQNAQVEAIFT
DKISKSAKRERVLKLFSDEKSTSLFSEFLKLIVGNQADFKKHFDLEEKAPLQFSKDTYDE
DLENLLGQIGDGFTDLFLVAKKLYDAILLSGILTVTDPSTKAPLSASMIERYESHQKDLA
ALKQFIKNNLPKRYNEVFSDQSKDGYAGYIDGKTTQEAFYKYIKNLLSKFEGADYFLDKI
EREDFLRKQRTFDNGSIPHQIHLQEMNAILRRQGEHYPFLKENREKIEKILTFRIPYYVG
PLARGNRDFAWLTRNSDQAIRPWNFEEIVDKASSAEEFINKMTNYDLYLPEEKVLPKHSL
LYETFAVYNELTKVKFIAEGLRDYQFLDSGQKKQIVNQLFKEKRKVTEKDIIHYLHNVDG
YDGIELKGIEKQFNASLSTYHDLLKIIKDKEFMDDPKNEEILENIVHTLTIFEDREMIKQ
RLAQYDSIFDEKVIKALTRRHYTGWGKLSAKLINGICDKQTGDTILDYLIDDGKINRNFM
QLINDDGLSFKEIIQKAQVVGKTDDVKQVVQELPGSPAIKKGILQSIKIVDELVKVMGYA
PESIVIEMARENQTTARGKKNSQQRYKRIEDSLKNLAPGLDSNILKENPTDNIQLQNDRL
FLYYLQNGKDMYTGKPLDIDQLSSYDIDHIIPQAFIKDDSIDNRVLTSSKDNRGKSDNVP
SLEVVQKRKAFWQQLLDSKLISERKFNNLTKAERGGLDERDKVGFIRRQLVETRQITKHV
AQILDASFNTEVNEKNQKIRTVKIITLKSNLVSNFRKEFELYKVREINDYHHAHDAYLNA
VVAKAILKKYPKLEPEFVYGDYQKYDLKRYISRFKPSKEIEKATEKYFFYSNLLNFFKEE
VHYADGIIVKRENIEYSKDTGEIAWNKEKDFATIKKVLSYPQVNIVKKTEIQTHGLDRGK
PKGLFNSNPSPKPSEDSKENLVPIKQGLDPRKYGGYAGISNSYAVLVKAIVEKGAKKQQK
TILEFQGISILDKINFENNKENYLLKKRYIEILSTITLPKYSLFEFPDGTRRRLASILST
NNKRGEIHKGNELVLPGKYTTLLYHAKNINKKLEPEHLEYVEKHRNDFAKLLECVLNFND
KYVGALKNGERIRQAFTDWETVDIEKLCFSFIGPENSKNAGLFELTSQGSASDFEFLGVK
IPRYRDYAPSSLLKATLIHQSITGLYETRIDLSKLGEDKRPAATKKAGQAKKKKGSYPYD
VPDYAYPYDVPDYAYPYDVPDYA (SEQ ID NO: 34).
Amino Acid Sequence of Adenine Deaminase, TadA8.13m
fused to the N-terminal of nickase Ssc2Cas9
(ABE-nSsc2Cas9, D9A mutant)
MPAAKRVKLDGSEVEFSHEYWMRHALTLAKRARDEREVPVGAVLVLNNRVIGEGWNRAIG
LHDPTAHAEIMALRQGGLVMQNYRLYDATLYVTFEPCVMCAGAMIHSRIGRVVFGVRNAK
TGAAGSLMDVLHHPGMNHRVEITEGILADECAALLCRFFRMPRRVFNAQKKAQSSTDGSS
GSETPGTSESATPESSGPKKKRKVGTMSDLVLGLAIGIGSVGVGILNKVTGEIIHKNSRI
FPAAQAENNLERRTNRQGRRLTRRKKHRRVRLNHLFEESGLITDFTKVSINLNPYQLRVK
GLTAELSNEELFIALKNMVKHRGISYLDDASDDGNSSVGDYAQIVKENSKQLETKTPGQI
QLERYQKYGQLRGDFTVEEDGRKHRLINVFPTSAYHAEALRILQTQQEFNPQITDEFINS
YLEILTGKRKYYHGPGNEKSRTDYGKYTTKKDAQGQYITLNNIFGILIGKCTFYPEEYRA
AKASYTAQEFNLLNDLNNLTVPTETKKLSEEQKYQIITYVKNEKAMGPAKLFKYIAKLLS
CDVADIKGYRIDKSDKAEIHTFEAYRKMKTLETLDVKKMAREELDKLAYVLTLNTEREGI
QEALDHEFADGTFSQEQVDELVQFRKANSSIFGKGWHSFSVKLMMELIPELYATSEEQMT
ILTRLGKQKTTSSSNKTKYIDEKQLTEEIYNPVVAKSVRQAIKIVNAAIKKYGDFDNIVI
EMARETNEDDEKKAIQKIQKANKAEKDAAMRKAANQYNGKAELPHSVFHGHKQLATKIRL
WHQQGERCLYTGKTISIHDLINNPNQFEIDHILPLSITFDDSLANKVLVYATANQEKGQR
TPYQALDSMDDAWSFRELKAFVRESKALSNKKKEYLLTEEDISKFDVRKKFIERNLVDTR
YASRVVLNALQEHFRTHKIDTKVSVVRGQFTSQLRRHWGIEKTRDTYHHHAVDALIIAAS
SQLNLWKKQKNTLVSYSEDQLLDIETGELISDDEYKESVFKAPYQHFVDTLKSKEFEDSI
LFSYQVDSKFNRKISDATIYATRKAKLDKEKKEYTYTLGKIKDIYALGTKTPSKTGFYKF
LDLYKKDKSQFLMYQKDRRTWDEVIEKILEQYRPFKEKDKNGKEVDENPFEKYRIENGPI
RKYSRKGNGPEIKSLKYYDNLLGRFVDITPSESKNPVALLSLNPWRTDVYYNTETRKYEF
LGLKYADLCFEKGGSYGISKVKYNKIREKEGIGKNSEFKFTLYKNDLILIKDTETNRQQI
FRFWSRTGKDNPKSFEKHKLELKPYEKTRFEKGEELKVLGKVPPSSNRLQKNMQIENLSI
YKVRTDVLGNQHIIKNEGDKPKLDFKRPAATKKAGQAKKKKGSYPYDVPDYAYPYDVPDY
AYPYDVPDYA (SEQ ID NO: 35).
Amino Acid Sequence of Adenine Deaminase, TadA8.13m
fused to the N-terminal of nickase Sor2Cas9
(ABE-nSor2Cas9, D9A mutant)
MPAAKRVKLDGSEVEFSHEYWMRHALTLAKRARDEREVPVGAVLVLNNRVIGEGWNRAIG
LHDPTAHAEIMALRQGGLVMQNYRLYDATLYVTFEPCVMCAGAMIHSRIGRVVFGVRNAK
TGAAGSLMDVLHHPGMNHRVEITEGILADECAALLCRFFRMPRRVFNAQKKAQSSTDGSS
GSETPGTSESATPESSGPKKKRKVGTMNGLVLGLAIGIASVGVGILEKNSGKIIHANSRI
FPAATADNNVERRKNRQARRLHRRKKHRGVRLQDIFEDYGLLTDFSKVSINLNPYRLRVD
GLDQQLTNEELFIALKNIVKRRGISYLDDASEDGGTVSSDYGKAVEENRKLLAEKTPGQI
QLERFEKYGQVRGDFTVVENGEKRRLINVESTSAYRKEAERILRKQQEFNSKITDEFIED
CLKILTGKRKYYHGPGNEKSRTDYGRFRTDGTTLDNIFGILIGKCTFYPNEYRASKASHT
AQEFNLLNDLNNLTVPTETKKLSEEQKKVIVEYAKEAKTLGASTLLKYIAKMIDASVDQI
SGYRVDVNNKPEMHTFEVYRKMQSLETISVGELSRNVLDELAHILTLNTEREGIEEAINT
KLKDSFSQDQVLELVQFRKNNSSLFSRGWHNFSLKLMMELIPELYETSEEQMTILTRLGK
QKSKETSKRTKYIDEKELTEEIYNPVVAKSVRQAIKIINEATKKYGIFDNIVIEMARENN
EEDAKKDYIKRQKANQDEKNAAMEKAAFQYNGKKELPDNIFHGHKELATKIRLWHQQGEK
CLYTGKNIPISDLIHNQYKYEIDHILPLSLSFDDSLSNKVLVLATANQEKGQRTPFQALD
SMDDGWSYREFKSYVKESKLLGNKKKEYLLTEEDISKIEVKQKFIERNLVDTRYSSRVVL
NALQDFYKAHKFDTTISVVRGQFTSQLRRKWGLEKSRETYHHHAVDALIIAASSQLRLWK
KQNNPLIAYKEGQFVDSETGEILSLSDDEYKELVFKAPYDHFVDTLRSKTFEDSILFSYQ
VDSKYNRKISDATIYATRKAKLDKDKSEETYVLGKIKDIYSQAGYDAFIKIYNKDKSKFL
MYHKDPQTFEKVIEEILRTYPSKELNDKNKEIPCNPFEKYRQENGPIRKYSKKGNGPEIK
CLKYYDNKLGNYIDITPDGSDNQVVLQSIAPWRTDVYYNHKTGKYEFLGLKYSDLYFEKG
TGKYKISKEKYDNIKKIEGVVETSEFKFTLYKNDLILIKDVEKGQEQLFRFLSRNNKGKH
QVQLKPMNKSDFEKGEKLIDIFGTVPNSTTQCVKGLNKSNISIFKVKTDVLGKKHIIKKE
GDEPKLKFKRPAATKKAGQAKKKKGSYPYDVPDYAYPYDVPDYAYPYDVPDYA (SEQ
ID NO: 36).
Amino Acid Sequence of Adenine Deaminase, TadA8.13m
fused to the N-terminal of nickase SorCas9
(nSorCas9-ABE8, D9A mutant)
MPAAKRVKLDGSEVEFSHEYWMRHALTLAKRARDEREVPVGAVLVLNNRVIGEGWNRAIG
LHDPTAHAEIMALRQGGLVMQNYRLYDATLYVTFEPCVMCAGAMIHSRIGRVVFGVRNAK
TGAAGSLMDVLHHPGMNHRVEITEGILADECAALLCRFFRMPRRVFNAQKKAQSSTDGSS
GSETPGTSESATPESSGPKKKRKVGTMNGLVLGLAIGIASVGVGILEKNSGKIIHANSRI
FPAATADNNVERRKNRQARRLHRRKKHRGVRLQDIFEDYGLLTDFSKVSINLNPYRLRVD
GLDQQLTNEELFIALKNIVKRRGISYLDDASEDGGTVSSDYGKAVEENRKLLAEKTPGQI
QLERFEKYGQVRGDFTVVENGEKRRLINVESTSAYRKEAERILRKQQEFNSKITDEFIED
CLKILTGKRKYYHGPGNEKSRTDYGRFRTDGTTLDNIFGILIGKCTFYPNEYRASKASHT
AQEFNLLNDLNNLTVPTETKKLSEEQKKVIVEYAKEAKTLGASTLLKYIAKMIDASVDQI
SGYRVDVNNKPEMHTFEVYRKMQSLETISVGELSRNVLDELAHILTLNTEREGIEEAINT
KLKDSFSQDQVLELVQFRKNNSSLFSRGWHNFSLKLMMELIPELYETSEEQMTILTRLGK
QKSKETSKRTKYIDEKELTEEIYNPVVAKSVRQAIKIINEATKKYGIFDNIVIEMARENN
EEDAKKDYIKRQKANQDEKNAAMEKAAFQYNGKKELPDNIFHGHKELATKIRLWHQQGEK
CLYTGKNIPISDLIHNQYKYEIDHILPLSLSFDDSLSNKVLVLATANQEKGQRTPFQALD
SMDDGWSYREFKSYVKESKLLGNKKKEYLLTEEDISKIEVKQKFIERNLVDTRYSSRVVL
NALQDFYKAHKFDTTISVVRGQFTSQLRRKWGLEKSRETYHHHAVDALIIAASSQLRLWK
KQNNPLIAYKEGQFVDSETGEILSLSDDEYKELVFKAPYDHFVDTLRSKTFEDSILFSYQ
VDSKYNRKISDATIYATRKAKLDKDKSEETYVLGKIKDIYSQAGYDAFIKIYNKDKSKFL
MYHKDPQTFEKVIEEILRTYPSKELNDKNKEIPCNPFEKYRQENGPIRKYSKKGNGPEIK
CLKYYDNKLGNYIDITPDGSDNQVVLQSIAPWRTDVYYNHKTGKYEFLGLKYSDLYFEKG
TGKYKISKEKYDNIKKIEGVVETSEFKFTLYKNDLILIKDVEKGQEQLFRFLSRNNKGKH
QVQLKPMNKSDFEKGEKLIDIFGTVPNSTTQCVKGLNKSNISIFKVKTDVLGKKHIIKKE
GDEPKLKFKRPAATKKAGQAKKKKGSYPYDVPDYAYPYDVPDYAYPYDVPDYA (SEQ
ID NO: 37).
Amino Acid Sequence of Adenine Deaminase, TadA8.13m
fused to the N-terminal of nickase SwaCas9
(nSwaCas9-ABE8, D10A mutant)
MPAAKRVKLDGSEVEFSHEYWMRHALTLAKRARDEREVPVGAVLVLNNRVIGEGWNRAIG
LHDPTAHAEIMALRQGGLVMQNYRLYDATLYVTFEPCVMCAGAMIHSRIGRVVFGVRNAK
TGAAGSLMDVLHHPGMNHRVEITEGILADECAALLCRFFRMPRRVFNAQKKAQSSTDGSS
GSETPGTSESATPESSGPKKKRKVGTMKEKYILGLALGITSVGYGIINFETKKIIDAGVR
LFPEANVDNNEGRRSKRGSRRLKRRRIHRLDRVKSLLTEYNLINREQIPTSNNPYQIRVK
GLSEILSKDELAIALLHLAKRRGIHNINVSSEDEDASNELSTKEQINRNNKLLKNKYVCE
VQLQRLKEGQIRGEKNRFKTTDILKEIDQLLKVQKDYHNLDIDFINQYKEIVETRREYFE
GPGQGSPFGWNGDLKKWYEMLMGHCTYFPQELRSVKYAYSADLFNALNDLNNLIIQRNDS
EKLEYHEKYHIIENVFKQKKKPTLKQIAKEIGVNPEDIKGYRITKSGTPQFTEFKLYHDL
KSIVFDKSILENEAILDQIAEILTIYQDEESIKEELNKLPEILNEQDKAEIAKLTGYNVT
HRLSLKCIHLINEELWQTSRNQMEIFNYLNIKPNKVDLSEQNKIPKDLVDEFILSPVVKR
TFIQSINVINKVIEKYGIPEDIIIELARENNSDDRKKFINNLQKKNEATRKRINEIIGQT
GNQNGKRIVEKIRLHDQQEGKCLYSLESIPLMDLLNNPQNYEVDHIIPRSVAFDNSIHNK
VLVKQIENSKKGNRTPYQYLNSSDANLSYNQFKQHILNLSKSKDRISKKKKDYLLEERDI
NKFEVQKEFINRNLVDTRYATRELTSYLKAYFSANNMDVKVKTINGSFTNHLRKVWRFDK
YRNHSYKHHAEDALIIANADFLFKENKKLQNANKILEKPTIENDTQKVTVEKEEDYNNMF
ETPKLVEDIKQYRDYKFSHRVDKKPNRQLIKDTLYSTRMKDEHNYIVQTITDIYGKDNTN
LKKQFNKNPEKFLMYQNDPKTFEKLSIIMKQYSDEKNPLAKYYEETGEYLTKYSKKNNGP
IVKKIKLLGNKVGNHLDVTNKYENSTKKLVKLSIKNYRFDVYLTEKGYKFVTIAYLNVFK
KDNYYYIPKDLYQELKAKKKIKDTDQFIASFYKNDLIKLNGDLYKIIGVNSDDRNIIELD
YYDIKYKDYCEINNIKGEPRIKKTIGKKTESIEKLTTDVLGNLYLHTTEKAPQLIFKRGL
KRPAATKKAGQAKKKKGSYPYDVPDYAYPYDVPDYAYPYDVPDYA (SEQ ID NO:
38).
Amino Acid Sequence of Adenine Deaminase, TadA8.13m-
nickase fused to the N-terminal of nickase SscCas9
(ABE-n SscCas9, D9A mutant)
MPAAKRVKLDGSEVEFSHEYWMRHALTLAKRARDEREVPVGAVLVLNNRVIGEGWNRAIG
LHDPTAHAEIMALRQGGLVMQNYRLYDATLYVTFEPCVMCAGAMIHSRIGRVVFGVRNAK
TGAAGSLMDVLHHPGMNHRVEITEGILADECAALLCRFFRMPRRVFNAQKKAQSSTDGSS
GSETPGTSESATPESSGPKKKRKVGTMKRNYILGLAIGITSVGYGIIDYETRDVIDAGVR
LFKEANVENNEGRRSKRGARRLKRRRRHRLQRVKKLLFDYNLLTDHSELSGINPYEARVK
GLSQKLSEVEFSAALLHLAKRRGVHNVNEVEEDTGNELSTKEQISRNSKALEEKYVAELQ
LERLKTDGEVRGPNNRFKTSDYVKEAKQLLKVQKAYHQLDQSFIDTYIDLLETRRTYYEG
PGEGSPFGWKDIKEWYEMLMGHCTYFPEELRSVKYAYNADLYNALNDLNNLVIARDENEK
LEYYEKFQIIENVFKQKKKPTLKQIAKELLVNEEDIKGYRVTSIGKPEFTNFKIYHDIKG
ITERKEVLENAELLDQIAEILTIYQSSEDVQEELANLNSELTQEEIEQISNLKGYTGTHN
LSLKAINLILDELWHTNDNQMAIFNRLKLVPKKVDLSQQKEIPTTLVDDFILSPVVKRSF
IQSIKVINAIIKKYGLPNDIIIELAREKNSKDAQKMINEMQKRNRQMNERIEEIIRTTGK
ENAKYLIEKIKLHDMQEGKCLYSLESIPLEDLLNNPFNYEVDHIIPRSVSFDNSFNNKVL
VKQEENSKKGNRTPFQYLSSSDSKISYETFKKHILNLAKGKGRISKTKKEYLLEERDINR
FSVQKDFINRNLVDTRYATRELMNLLRSYFRVNNLDVKVKSINGGFTSFLRRKWKFKKER
NKGYKHHAEDALIIANADFIFKEWKKLDKAKKVMENQTVEEKQAESMPEIETEQEYKEIF
ITPHQIQHIKGFKDYKYSHRVDKKPNRELINDTLYSTRKDDKGNTLIVNNLNGLYDKDND
KLKKLMNKSPEKLLMYHHDPQTYQKLKLIMEQYGDEKNPLYKYYEETGNYLTKYSKKDNG
PVIKKIKYYGKNLKAHLDITDDYPNSRNKVVKLSVKPYRFDVYLDNDIYKFVTVKNLDVI
KKEDYYEVNSKCYKEAKKLKKISDQAEFIASFYNNDLIKINGELYRVIGVNNDLLNRIEV
NMINITYREYLENMNDKRPPRIIKTIASKTQSIKKYSTDILGNLYEVKSKQKPQMIMKGK
RPAATKKAGQAKKKKGSYPYDVPDYAYPYDVPDYAYPYDVPDYA (SEQ ID NO:
39).
Amino Acid Sequence of Adenine Deaminase, TadA8.13m-
nickase fused to the N-terminal of nickase SgaCas9
(n SgaCas9-ABE, D9A mutant)
MPAAKRVKLDGSEVEFSHEYWMRHALTLAKRARDEREVPVGAVLVLNNRVIGEGWNRAIG
LHDPTAHAEIMALRQGGLVMQNYRLYDATLYVTFEPCVMCAGAMIHSRIGRVVFGVRNAK
TGAAGSLMDVLHHPGMNHRVEITEGILADECAALLCRFFRMPRRVFNAQKKAQSSTDGSS
GSETPGTSESATPESSGPKKKRKVGTMTNGKILGLAIGIASVGVGIIEAKTGKVVHANSR
LFSAANAENNTERRGFRGSRRLNRRKKHRVKRVRDLFEKHEIVTDFRNLNLSPYELRVKG
LTEQLTNEELFAALRTISKRRGISYLDDAEDDSTGSTDYAKSIDENRRLLKNKTPGQIQL
ERLEKYGQLRGNFTVYDENGEAHRLINVESTSDYEKEARKILETQADYNKKITAEFIDDY
VEILTQKRKYYHGPGNEKSRTDYGRFRTDGTTLENIFGILIGKCNFYPDEYRASKASYTA
QEYNFLNDLNNLKVPTETGKLPTEQKESLVEFAKNTATLGPSKLLKEIAKILDCKVDEIK
GYREDDKGKPDLHTFEPYRKLKFNLESINIDDLSREVIDKLADILTLNTEREGIEDAIKR
NLPNQFTEEQISEIIKVRKSQSTAFNKGWHSFSAKLMNELIPELYATSDEQMTILTRLEK
FKVNKKSSKNTKTIDEKEVTDEIYNPVVAKSVRQTIKIINAAVKKYGDFDKIVIEMPRDK
NADDEKKFIDKRNKENKKEKDDALKRAAYLYNSSDKLPDEVFHGNKQLETKIRLWYQQGE
RCLYSGKPIPIHDLVHNSNNFEIDHILPLSLSFDDSLANKVLVYDWANQEKGQKTPYQVI
DSMDAAWSFREMKDYVLKQKGLGKKKRDYLLTTENIDKIEVKKKFIERNLIDTRYASRVV
LNSLQSALRELCKDTKVSVIRGQFTSQLRRKWKIDKSRETYHHHAVDALIIAASSQLKLW
EKQDNLMFIDYGNNQVVDKETGEILSVSDDEYKELVFQPPYQGFVNTISSKGFEDEILFS
YQVDSKYNRKVSDATIYSTRKAKIGKDKKEETYVLGKIKDIYSQNGFDTFIKKYNKDKTQ
FLMYQKDPLTWENVIEVILRDYPTTKKSEDGKNDVKCNPFEEYRRENGLICKYSKKGKGT
PIKSLKYYDKKLGNCIDITPEESRNKVILQSINPWRADVYFNPETLKYELMGLKYSDLSF
EKGTGKYHISQEKYDAIKEKEGIGKKSEFKFTLYRNDLILIKDTASGEQEIYRFLSRTMP
NVNHYAELKPYDKEKFDGGQELMEVFGKVANGGQCLKSLNKSNISIYKVRTDVLGNKYFV
KKEGDKPKLNFKNNKKKRPAATKKAGQAKKKKGSYPYDVPDYAYPYDVPDYAYPYDVPDY
A (SEQ ID NO: 40).
Amino Acid Sequence of Adenine Deaminase, TadA8.13m-
nickase fused to the N-terminal of nickase LkuCas9
(n SgaCas9-ABE, D13A mutant)
MPAAKRVKLDGSEVEFSHEYWMRHALTLAKRARDEREVPVGAVLVLNNRVIGEGWNRAIG
LHDPTAHAEIMALRQGGLVMQNYRLYDATLYVTFEPCVMCAGAMIHSRIGRVVFGVRNAK
TGAAGSLMDVLHHPGMNHRVEITEGILADECAALLCRFFRMPRRVFNAQKKAQSSTDGSS
GSETPGTSESATPESSGPKKKRKVGTMKRVNEDYILGLAIGTNSCGWAVTDKKNNLLKLR
GKTAIGSHLFEEGHTAADRRGFRTTRRRLKRRKWRLRLLEEIFAEPMAKVDPGFFVRLHQ
SWVSPLDKDRKKYNAIVFPTAKEDQAFYKHYATIYHLRDELMTQDRQFDLREIFLAIHHI
VKYRGNFLQDTPVKDFEASKIEVGPILSHINNAFAEKIVEDQDPIELNVANAADIEDVIR
GKDAEKTVYKLDKVKKIAKLLTDSTAKEEKNVAKQIANAIMGYKTQFETILDKEIDKSDK
AQWEFKLSDADADDKLDALLPDLDETDQTVVAEIEKLFSAITLSTIVDENKSLSQSMVEK
YKKHKKDYKKLKKYINTLQDQTKAKKLLLAYDLYVNNRHGRLLEAKKTFEDKKKKKALTK
DEFYKIVKDNLDDSDLAHEIQQEIAADNFMPKQRTNSNGVIPFQLHQIELDKIIANQGKY
YPFLAAENPVEDHRKQAPYKLDELVRFRVPYYVGPMITADEQEKTSGKSFAWMVRKEDGQ
ITPWNFEQKVDRQESANKFIKRMTIKDTYLLSEDVLPANSLLYQRFEVLNELNNIRINGS
RISVDLKQQIFNDLFEEKKTVTEKSLTSYLKQNLHLPTVEIKGLADPTKFNSSLASYYHL
KSLHVFDKELADPQYQKDFEKIIEYSSIFEDKKIFQDKLHAEFKWLTPEQFKAISTWRLQ
GWGRLSRKLLVELHDTNGQNIMEQLWDSQKNFMQIVTEPDFKDAIAKENQNVTRANGVEE
ILADAYTSPANKKAIRQVVKVVADVVKAAGGKKPAQFAIEFTRDPDKNPQLSHIRGTKLL
KAYQETAGELVDQKLTDSLKEAMTSRKLLKDKYFLYFMQAGRDAYTGQKINIDEVSTNYQ
IDHILPQSFIKDDSFDNRVLTATPLNAEKSDDVPYKRFANNYVSDMKMTVGEMWKHWQKA
GIINKHKLGNLLLDPDRLNKFQKSGFINRQLVETSQIIKLVSVILQNKYPDAEIITVKAG
DNSALRQRLNLYKSRDVNDYHHAIDAYLSIICGNFLYQVYPKYRPYFVYGKYKKFSQDPD
LQKEVIKHFKGFTFMWPLLQKDNSERKAPEKIKENNSDRIVFYKHPDIFDKLRKAYNYKY
MLVSRETTTENSGLFDVTIYPRGERDLAKTRKLIPKSNGLDPKIYGGYSGNTDAYMVIVK
IDKGKESIYKVIGVPMRALASLNRAKKQGNYKEELHQVLEPQIMFDKNGKPKRSVKGFRI
IKDHVPFKQVVLDGDKKFMLNSSTYEINAKQLTLTPETMRIVTDNLKKGEDQDQLLVKAY
DEILQKVDQYLPLFDVNKFRNSLHLGRAKFLDLAVNDKKITLTNILNGLHDNLVTPDLKN
IGIKTPLGKLQVPSGIVLSSEAILIFQSPTGLFEKRVRIADLKRPAATKKAGQAKKKKGS
YPYDVPDYAYPYDVPDYAYPYDVPDYA (SEQ ID NO: 41).
Amino Acid Sequence of Adenine Deaminase, TadA8.13m-
nickase fused to the N-terminal of nickase SsuCas9
(ABE-SsuCas9, D10A mutant)
MPAAKRVKLDGSEVEFSHEYWMRHALTLAKRARDEREVPVGAVLVLNNRVIGEGWNRAIG
LHDPTAHAEIMALRQGGLVMQNYRLYDATLYVTFEPCVMCAGAMIHSRIGRVVFGVRNAK
TGAAGSLMDVLHHPGMNHRVEITEGILADECAALLCRFFRMPRRVFNAQKKAQSSTDGSS
GSETPGTSESATPESSGPKKKRKVGTMSNGKILGLAIGIASVGVGVIDAQTGEIIHASSR
IFPSANAANNAERRTFRGSRRLIRRKKHRIKRLDDLFNDFHINLDGEMSTDNPYVLRVKG
LSQKLTVEELYISIKNIMKRRGISYLDDAESDNEAGRSDYAKAIERNRQLLTSKTPGEIQ
LERLEKYGQLRGNFTIIDEEGQSQQIINVESTSDYVKEVEKILDCQKMYHKFISDEFCDK
LIELLREKRKYYVGPGNEKSRTDYGIYRTDGTTLENLFGILIGKCTFYPDQYRSSRASYT
AQEFNFLNDLNNLTVPTETKKLSQEQKEFLVNYAKETSVLGAGKILQQIAKLADCKVEDI
RGYRLDNKDKPELHTFETYRAMKGLVPLVDIGVLSREQLDILADILTLNTDFEGIREALK
KQLPNVFDEKQVKGLASFRKSKSQLFAKGWHNLSQKIMLEVIPELYATSDEQMTILTRLG
KFEKSSVAEYPSSINVDEITDEIYNPVVAKSIRQTIKIINASIKKWDEFDQIVIEMPRDR
NEDEEKKRIADGQKANAKEKADSILRAAELYCAGKVLPDYVYNGHNQLATKIRLWYQQGE
RCIYTGQPISIHDLIHNQNQYEIDHILPLSLTFDDSLSNKVLVLATANQEKAQRTPYNYL
KSATSAWSYREFKDYVTKRKGIGKKKCEYLTFEEDINGFEVRSKFIQRNLVDTRYASKVI
LNALQDYFKISGIQTKVSVVRGQFTSQLRHKWGIEKTRETYHHHAVDALIIAASSQLRLW
KKQESPLVVDYQEGRQVDLETGEILELTDEQYKELVYQPPYQGFVNTISSSAFDNEILFS
YQVDSKVNRKISDATIYATRNAQLGKDKTEGIYVLGKIKDIYTQAGYEAFLKRYTKDKTS
FLMYHKDLDTWEKVIEIILRDYREYDEKGKEIGNPFERYRRENGYVKKYSRKGNGTAIKS
LKYYDNKLGNHIDITPENSRNAVVLQSLKPWRTDVYFNKETGKYEFLGIKYSDLSFEKGT
GEYGISQEKYDSIKIAEGVAKKSIFKFTLYKQDLLFIKDIENNFGKLLRFTSKNDTSKHY
VELKPYDKNKFGTEEPLLPVLGNVAKSGQCIKGLNKSNISIYKVRTDILGYRHFIKQEGE
HPQLKFKKKRPAATKKAGQAKKKKGSYPYDVPDYAYPYDVPDYAYPYDVPDYA (SEQ
ID NO: 42).
Linker (underlined, no italics or bolding)
TadA8 (ABE) (italics and underlined)
Nickase mutation (bold and italics)
3xHA tag (italics), can be substituted with different tags

### EQUIVALENTS AND SCOPE

Those skilled in the art will recognize, or be able to ascertain using no more than routine experimentation, many equivalents to the specific embodiments of the invention described herein. The scope of the present invention is not intended to be limited to the above Description, but rather is as set forth in the following claims.

1. An engineered, non-naturally occurring Cas9 protein modified from Streptococcus equinus ATCC 33317 Cas9, Enterococcus hirae strain F1129E Cas9, Streptococcus equinus strain AG46 Cas9, Staphylococcus simulans strain 19 Cas9, Streptococcus intermedius B196 strain G1552, Streptococcus sanguinis SK330 Cas9, Streptococcus sp. C150 Cas9, Streptococcus oralis subsp. oralis strain RH_1735_08 Cas9, Streptococcus oralis SK313 Cas9, Staphylococcus warneri strain 691 Cas9, Staphylococcus sciuri strain SNUC 2430 Cas9, Streptococcus gallolyticus strain AM24-4 Cas9, Lactobacillus kullabergensis strain Biut2 Cas9, or Streptococcus suis strain LSS83 Cas9 is provided herein.

2. The Cas9 protein of claim 1, wherein the Streptococcus equinus ATCC 33317 Cas9 has at least 80% sequence identity to
(SEQ ID NO: 1)
MEKSYSIGLDIGTNSVGWSVITDDYKVPAKKMRVLGNTDKKYIKK
NLLGALLFDSGETAEATRLKRTARRRYTRRKNRLRYLQEIFAKEM
AKVDESFFQRLDESFLTDDDKTFDSHPIFGNKAEEDAYHQKFPTI
YHLRKHLADSQEKADLRLIYLALAHMIKFRGHFLYDNFNDDNFDW
RNIDIQKRYEEFIETYDSTLGESYLADISVDAASILEEKVSKTER
LENLLKYYPTEKKTTFFGNLIKLILGQQAKFKAIFNLEDEISLQF
ATPTYDEDLEELLGKIDNGDSYSELFVAAQNLYNTILLASFLKTD
NKSAKAPLSTSMIERYENHKKDLAKLKDFVKKNCPDQYHDIFRDK
SKNGYAGYIDNGVSQNDFYTFIGKCLEESLKKDKGAQYFLDKIDR
DDFLRKQRTFDNGAFPYQIHLQEMHAILRRQGDYYPFLKENQDKI
EKILTFRIPYYVGPLARKDSRFAWANYRSDEAITPWNFDEVVDKE
KSAEKFITRMTLNDLYLPEEKVLPKHSLIYETFTVYNELTNIKYV
NDQGNAIHFDSELKEKIFNQLFKENRKVSKKTLIDFLNNSEGIYT
DKLVGIDEEVKYLNASLGTYHDLKKILESFMDDEINEKIIEDIIQ
TLTLFEDIEMKRQRLQKYDDIFTPKQLKELARRNYTGWGRLSYKL
INGIRNKENNKTILDYLKNGNRNFMQLINDDRLSFKQIIIDARKI
EKLDNIESVVYNLPGSPAIKKGILQSIKIVDELVKVMGHNPDNIV
IEMARENQTTNQGKNRSQQRLKRLQDSMSNFKDSSISLKDVDNSD
LQNDRLFLYYIQNGKDMYTGEELDIDHLSDYDIDHIIPQSFIKDN
SIDNRVLTSSAKNRGKSDDVPGRDVVLKMKPFWKKLYDVKLISKR
KFDNLTKSEHGGLTESDKAGFIKRQLVETRQITKYVAQILDGRFN
TKRDDNNKVIRDVKVITLKSSLVSQFRKDFGFYKVREINDYHHAH
DAYLNAVVGTAILKKYPKLAPEFVYGEYKKCDVRKLIAKSGDKSE
IGKATAKYFFYSNLMNFFKRVIRYSNGMIVVRPVIEYSKDTGEIA
WDKEKDFKTVCKVLSCPQVNIVKKVEKQSHGLDRGKPKGFYNANP
SPKPKKGSKVNLVPIKANLNPKNYGGYAGISNSYAVLVDATIEKG
AKKKLTRIQEFQGISIIDREKYEKNKVEFLKGLGYKEIYSIITLP
KYSLFELADGSRRMLASILSTNNKRGEIHKGNELVLPAKYIPLLY
HANRIHNTFETGHREYVEKHIAEFKEIAEIILEFNNKYVNAKKNS
SIIEKALESFDSFSLDEICDSFVGKLKKNNTKKNSGLFELVSLGS
ASDFEFLETKVPRYRDYTPSSLLNATLIHQSITGLYETRIDLSKL
GEE

3. The Cas9 protein of claim 1, wherein the Enterococcus hirae strain F1129E Cas9 has at least 80% sequence identity to
(SEQ ID NO: 2)
MTKDYTIGLDIGTNSVGWAVLTDDYQLMKRKMSVHGNTEKKKIKK
NFWGARLFDEGQTAEFRRTKRTNRRRLARRKYRLSKLQDLFAEEL
CKQDDCFFVRLEESFLVPEEKQYKPASIFPTLEEEKEYYQKYPTI
YHLRQKLVDSTEKEDLRLVYLALAHLLKYRGHFLFEGDLDTENTS
IEESFRVFLEQYSKQSDQPLIVHQPVLTILTDKLSKTKKVEEILK
YYPTEKINSFFAQCLKLIVGNQANFKRIFDLEAEVKLQFSKETYE
EDLESLLEKIGDEYLDIFLQAKKVHDAILLSEIISSTVKHTKAKL
SSGMVERYERHKADLAKFKQFVKENVPQKSTAFFKDTTKNGYAGY
IKGKTTQEEFYKFVKKELSGVVGSEPFLEKIDQETFLLKQRTYTN
GVIPHQVHLIELKAIIDQQKQHYPFLEEAGPKIIALFKFRIPYYV
GPLAKEQEASSFAWIERKTAEKIHPWNFSEVVDIEKSAMRFIQRM
TKQDTYLPTEKVLPKNSLLYQKYMIFNELTKVSYKDERGVKQYFS
GDEKQQIFKQLFQKERGKITVKKLQNFLYTHYHIENAQIFGIEKA
FNASYSTYHDFMKLAKTNQKAMQEWLEQPEMEPIFEDIVKILTIF
EDRQMIKHQLSKYQEVFGEKLLKEFARKHYTGWGRFSAKLIHGIR
DRKTNKTILDYLINDDDVPANRNRNLMQLINDEHLSFKEEIAKAT
VFSKHKSLVDVIQDLPGSPAIKKGIWQSLKIVEELIAIIGYKPKN
IVIEMARENQKTHRTKPRLKALENGLKQIGSTLLKEQPTDNKALQ
KERLYLYYLQNGRDMYTGEPLEIENLHQYEVDHIIPRSFIVDNSI
DNKVLVARKQNQKKRDDVPKKQIVNEQRIFWNQLKEAKLISPKKY
AYLTKIELTPEDKARFIQRQLVETRQITKHVANILHQSFHQEEEG
TDCDGVQIITLKATLTSQFRQTFGLYKVREINPHHHAHDAYLNGF
IANVLLKRYPKLAPEFVYGKYVKYSLARENKATAKKEFYSNILKF
FESDEPFCDENGEIYWEKSHHLPRIKKVLSSHQVNVVKKVEQQKG
GFYKETVNSKEKPDKLIERKNNWDVTKYGGFGSPVIAYAIAFVYA
KGKTQKKTRAIEGITIMEQAAFEKDPTTFLKEKGFPQVTEFIKLP
KYTLFEFDNGRRRFLASHKESQKGNPFILSDQLVTLLYHAQHYDK
ITYQESFDYVNTHLSDFSAILTEVLAFAEKYTLADKNIERIQELY
EENKYGEISMIAQSFLQLLQFNAIGAPADFKFFGVTIPRKRYTSL
TEIWDATIIYQSVTGLYETRIRMGDLWAGEQ.

4. The Cas9 protein of claim 1, wherein the Streptococcus equinus strain AG46 Cas9 has at least 80% sequence identity to
(SEQ ID NO: 3)
MTNGKILGLDIGVASVGVGIIEAKTGKVIHANSRLFSAANAENNA
ERRGFRGARRLTRRKKHRVKRVRDLFEKYDISTDFRNLNLNPYEL
RVKGLSEQLTNEELFAALRTIAKRRGISYLDDAEDDSTGSSDYAK
SIDENRRLLKSMTPGQIQLERLEKYGQLRGNFTVYDENGEAHRLI
NVESTSDYKNEARKILETQSNYNKQITDEFIEDYIEILTQKRKYY
HGPGNEKSRTDYGRFRTDGTTLENIFGILIGKCSFYPEEYRASKA
SYTAQEFNFLNDLNNLKVPTETGKLSTEQKEYLVDFAKNSKTLGA
SKLLKEIAKLVDGDVKDISGYREDSKGKPDLHTFEPYRKLKFNLT
TVDIDNLSRDILDKLANILTLNTEREGIEDAINRNLPEQFTKEQI
SEIVQIRKSQSSAFNKGWHSFSAKLMNELIPELYATSEEQMTILT
RLEKFKATKKSSKNTKTIDEKEITDEIYNPVVAKSVRQTIKIINA
AVKKYGDFDKIVIEMPRDKNADDEKKFIDKKNKENKKEKDDSLKR
AAYLYNGTDKLPDDVFHGNKQLATKIRLWYQQGERCLYSGKPILI
QDLVHNSNNFEIDHILPLSLSFDDSLANKVLVYAWTNQEKGQKTP
YQVIDSMDAAWSFREMKDYVLKQKGIGKKKREYLLTTENIDKIEV
KKKFIERNLVDTRYASRVVLNSLQTALKELGKDTKVSVVRGQFTS
QLRRKWKIDKSRETYHHHAVDALIIAASSQLKLWQKHENLMFENY
GENQVVNKETGEILSISDDEYKELVFQPPYQGFVNTISSKAFEDE
ILFSYQVDSKFNRKVSDATIYSTRKAKLGKDKKEETYVLGKIKDI
YSQDGFDTFIKRYKKDKTQFLMYQKDPLTWENVIEVILRDYPTTK
KSEDGKNDVKCNPFEEYRRENGLICKYSKKGKGTPIKSLKYYDKK
LGNHISITPKESKNDVVLQSLNPWRADLYFNPDTLKYELMGLKYS
DLSFEKGTGKYHISQEKYDEIKEKEGIGQNSEFKFTLYRNDLILI
KDTESGEQEIYRFLSRTMPNVKHYVELKPYDKEKFNGGQELIKSL
GEADKVGRCLKGLSKPGISIYKVRTDVLGNKFFVKKEGDKPKLDF
KNNKK

5. The Cas9 protein of claim 1, wherein the Staphylococcus simulans strain 19 Cas9 has at least 80% sequence identity to
(SEQ ID NO: 4)
MNNSYILGLDIGITSVGYGIIEYETRDVIDAGVRLFKEANVENNE
GRRSKRGARRLKRRRRHRLQRVKKMLFDYKLLNEDSEISGINPYE
ARVKGLSEKLSDEEFSAALLHLAKRRGVHNVSDVEEDTGNELSTK
EQIARNNKALEDKYVAELQLERLKEQGEVRGAANRFKTSDYIKEA
KQLLKTQSDYHKIDETFIETYISLLETRRTYYEGPGEGSPFGWKD
IKEWYEMLMGHCTYFPEELRSVKYAYNADLYNALNDLNNLVIARD
ENEKLEYYEKFQIIENVFKQKKKPTLKQIAKEILVNEEDIKGYRV
TSTGKPEFTNFKIYHDIKGITSRKEILENADLLNQIAEILTIYQS
AEDIQEELAELEPKLTQEEIEQISNLTGYTGTHRLSLKAINLILD
ELWNTSDNQMTIFNRLKLVPKKVDLSQQKEIPTSLVDDFILSPVV
KRSFIQSIKVINAIIKKFGLPKDIIIELAREKNSKEAQKFINEMQ
KRNRQTNERIEKIIKETGKEKAKFLIEKIKLHDMQEGKCLYSLES
IPLEDLLNNPYHYEVDHIIPRSVSFDNSFNNKVLVKQEENSKKGN
RTPFQYLSSSDAKISYETFKKHILNLSKGKGRVSKKKKEYLLEER
DINRFSVQKDFINRNLVDTRYATRELMNLLRSYFRVNDLDVKVKS
INGGFTSFLRTKWKFKKERNQGYKHHAEDALVIANADFIFKEWKK
LDTTNKVMENQTVEEQQAENMPGIETDDEYKEIFVIPRQIQSIKD
FKDYKYSHRVDKKPNRELVNDTLYSTRKDDKGNTLIINNIKGLYD
KDNDKLKNLIKKSPEKLLMYHHDPQTYQKLKTIMEQYSNEKNPLY
KYHEETGNYLTKYSKKDNGPIIKKVKYYGKKLNAHLDITNDYSNS
QNKIVKLSLKPYRFDVYLDNGGYKFVTVKNLDVIKKEGFFKIDSN
AYEKAKSEKKIDENAVFIASFYNNDLIKIDGELYRIVGVNNDTRN
VVELNMIPITYKEYLENINDKRTPRILKTISQKTYSIEKYSTDIL
GNLYKVKSKKKPQMIMKG

6. In some embodiments, the Streptococcus intermedius B196 strain G1552 Cas9 protein has at least 80% sequence identity to
(SEQ ID NO: 5)
MNGLVLGLDIGIASVGVGILNKETGEIIHVNSRIFPAATADSNVE
RRGFRQGRRLGRRKKHRSARLNDLFEEFGFITDFSAVPLNLNPYA
LRVKGLSEELTNEELFIALKNIIKRRGISYLDDASEDGETASNEY
GKAVEENRKLLADKTPGQIQLERFEKYGQVRGDFTVVENGENHRL
INVESTSAYKKEAERILRRQQEFNVRISDEFIEAYLTILTGKRKY
YHGPGNEKSRTDYGRFRTDGTTLDNIFGILIGKCTFYPDEYRAAK
ASYTAQEFNLLNDLNNLTVPTETKKLRPEQKRQIVEYARTAKTLG
TPTLLKYIAKLVDGSIDDIKGYRIDKSDKPEMHTFDAYRKMRTLE
LVDVDILSRETLDDLAYILTLNTESEGILEALNSKMPGTFTKEQI
DELIQFRKKNSAVFGKGWHNFSLKLMNELISELYETSEEQMTILT
RLGKQRSREISKRTKYIDEKELTDEIYNPVVAKSVRQAIKIINLA
TKKYGIFDNIVIEMARESNEDDEKKAIQNVQKANEDEKKAAMEKA
ADLYNGKKELPDSIFHGHKELATKIRLWHQQGERCLYTGKNISIH
DLIHNPHQYEIDHILPLSLSFDDGLANKVLVLATANQEKGQRTPF
QALDSMDDAWSYIEFKQYVRNSKSLSNKKKDYLLTEEDISKIEVK
QKFIERNLVDTRYSSRVVLNTLQEFYKTNDFDTKISVVRGQFTSQ
LRRKWKIEKSRDTYHHHAVDALIIAASSQLRLWKKQNNPLISYRE
GQLVDPETGEILSLTDDEYKELVFRPPYDYFVDTLKSKSFEDSIL
FSYQVDSKYNRKISDATIYGTRKAQLGKDKQEETYVLGKIKDIYS
QKGYEDFIKRYKKDTTQFLMYHKDPQTFAKVIEEILKTYPDKELN
EKGKEIPCNPFEKYRQENGPIRKYSKKGKGPEIKSLKYYDNKLGN
HIDITPVNSQNQVVLQSLKPWRTDVYFNPQTSKYELMGLKYSDLR
FEKGSGSYGISPEKYNKVKAKEGVDEDSEFKFTLYKNDLILIKDT
ETGEQQLFRYGSRNDTSKHYVELKPYEKAKFEGNQQLMNLLGTVA
KGGQCLKGINKPNLSIYKVKTDVLGNKHFIKKEGDQPQLNFKKKI

7. The Cas9 protein of claim 1, wherein the Streptococcus sanguinis SK330 Cas9 has at least 80% sequence identity to
(SEQ ID NO: 6)
MENKNYSIGLDIGTNSVGWAVITDDYKVPSKKMKVFGNTDKHFIK
KNLIGALLFDEGATAEDRRLKRTARRRYTRRKNRLRYLQEIFSEE
ISKLDSSFFHRLDDSFLVPKDKRGSKYPIFATLEEEKEYHKKFPT
IYHLRKHLADSKEKTDLRLIYLALAHMIKYRGHFLYEESFDIKNN
DIQKIFNEFISIYDNTFEGSSLSGQNAQVEAIFTDKISKSAKRER
VLKLFSDEKSTSLFSEFLKLIVGNQADFKKHFDLEEKAPLQFSKD
TYDEDLENLLGQIGDGFTDLFLVAKKLYDAILLSGILTVTDPSTK
APLSASMIERYESHQKDLAALKQFIKNNLPKRYNEVFSDQSKDGY
AGYIDGKTTQEAFYKYIKNLLSKFEGADYFLDKIEREDFLRKQRT
FDNGSIPHQIHLQEMNAILRRQGEHYPFLKENREKIEKILTFRIP
YYVGPLARGNRDFAWLTRNSDQAIRPWNFEEIVDKASSAEEFINK
MTNYDLYLPEEKVLPKHSLLYETFAVYNELTKVKFIAEGLRDYQF
LDSGQKKQIVNQLFKEKRKVTEKDIIHYLHNVDGYDGIELKGIEK
QFNASLSTYHDLLKIIKDKEFMDDPKNEEILENIVHTLTIFEDRE
MIKQRLAQYDSIFDEKVIKALTRRHYTGWGKLSAKLINGICDKQT
GDTILDYLIDDGKINRNFMQLINDDGLSFKEIIQKAQVVGKTDDV
KQVVQELPGSPAIKKGILQSIKIVDELVKVMGYAPESIVIEMARE
NQTTARGKKNSQQRYKRIEDSLKNLAPGLDSNILKENPTDNIQLQ
NDRLFLYYLQNGKDMYTGKPLDIDQLSSYDIDHIIPQAFIKDDSI
DNRVLTSSKDNRGKSDNVPSLEVVQKRKAFWQQLLDSKLISERKE
NNLTKAERGGLDERDKVGFIRRQLVETRQITKHVAQILDASFNTE
VNEKNQKIRTVKIITLKSNLVSNFRKEFELYKVREINDYHHAHDA
YLNAVVAKAILKKYPKLEPEFVYGDYQKYDLKRYISRFKPSKEIE
KATEKYFFYSNLLNFFKEEVHYADGIIVKRENIEYSKDTGEIAWN
KEKDFATIKKVLSYPQVNIVKKTEIQTHGLDRGKPKGLFNSNPSP
KPSEDSKENLVPIKQGLDPRKYGGYAGISNSYAVLVKAIVEKGAK
KQQKTILEFQGISILDKINFENNKENYLLKKRYIEILSTITLPKY
SLFEFPDGTRRRLASILSTNNKRGEIHKGNELVLPGKYTTLLYHA
KNINKKLEPEHLEYVEKHRNDFAKLLECVLNFNDKYVGALKNGER
IRQAFTDWETVDIEKLCFSFIGPENSKNAGLFELTSQGSASDFEF
LGVKIPRYRDYAPSSLLKATLIHQSITGLYETRIDLSKLGED

8. The Cas9 protein of claim 1, wherein the Streptococcus sp. C150 Cas9 has at least 80% sequence identity to
(SEQ ID NO: 7)
MSDLVLGLDIGIGSVGVGILNKVTGEIIHKNSRIFPAAQAENNLE
RRTNRQGRRLTRRKKHRRVRLNHLFEESGLITDFTKVSINLNPYQ
LRVKGLTAELSNEELFIALKNMVKHRGISYLDDASDDGNSSVGDY
AQIVKENSKQLETKTPGQIQLERYQKYGQLRGDFTVEEDGRKHRL
INVFPTSAYHAEALRILQTQQEFNPQITDEFINSYLEILTGKRKY
YHGPGNEKSRTDYGKYTTKKDAQGQYITLNNIFGILIGKCTFYPE
EYRAAKASYTAQEFNLLNDLNNLTVPTETKKLSEEQKYQIITYVK
NEKAMGPAKLFKYIAKLLSCDVADIKGYRIDKSDKAEIHTFEAYR
KMKTLETLDVKKMAREELDKLAYVLTLNTEREGIQEALDHEFADG
TFSQEQVDELVQFRKANSSIFGKGWHSFSVKLMMELIPELYATSE
EQMTILTRLGKQKTTSSSNKTKYIDEKQLTEEIYNPVVAKSVRQA
IKIVNAAIKKYGDFDNIVIEMARETNEDDEKKAIQKIQKANKAEK
DAAMRKAANQYNGKAELPHSVFHGHKQLATKIRLWHQQGERCLYT
GKTISIHDLINNPNQFEIDHILPLSITFDDSLANKVLVYATANQE
KGQRTPYQALDSMDDAWSFRELKAFVRESKALSNKKKEYLLTEED
ISKFDVRKKFIERNLVDTRYASRVVLNALQEHFRTHKIDTKVSVV
RGQFTSQLRRHWGIEKTRDTYHHHAVDALIIAASSQLNLWKKQKN
TLVSYSEDQLLDIETGELISDDEYKESVFKAPYQHFVDTLKSKEF
EDSILFSYQVDSKFNRKISDATIYATRKAKLDKEKKEYTYTLGKI
KDIYALGTKTPSKTGFYKFLDLYKKDKSQFLMYQKDRRTWDEVIE
KILEQYRPFKEKDKNGKEVDENPFEKYRIENGPIRKYSRKGNGPE
IKSLKYYDNLLGRFVDITPSESKNPVALLSLNPWRTDVYYNTETR
KYEFLGLKYADLCFEKGGSYGISKVKYNKIREKEGIGKNSEFKFT
LYKNDLILIKDTETNRQQIFRFWSRTGKDNPKSFEKHKLELKPYE
KTRFEKGEELKVLGKVPPSSNRLQKNMQIENLSIYKVRTDVLGNQ
HIIKNEGDKPKLDF

9. The Cas9 protein of claim 1, wherein the Streptococcus oralis subsp. oralis strain RH_1735_08 Cas9 has at least 80% sequence identity to
(SEQ ID NO: 8)
MNGLVLGLDIGIASVGVGILEKNSGKIIHANSRIFPAATADNNVE
RRKNRQARRLHRRKKHRGVRLQDIFEDYGLLTDFSKVSINLNPYR
LRVDGLDQQLTNEELFIALKNIVKRRGISYLDDASEDGGTVSSDY
GKAVEENRKLLAEKTPGQIQLERFEKYGQVRGDFTVVENGEKRRL
INVESTSAYRKEAERILRKQQEFNSKITDEFIEDCLKILTGKRKY
YHGPGNEKSRTDYGRFRTDGTTLDNIFGILIGKCTFYPNEYRASK
ASHTAQEFNLLNDLNNLTVPTETKKLSEEQKKVIVEYAKEAKTLG
ASTLLKYIAKMIDASVDQISGYRVDVNNKPEMHTFEVYRKMQSLE
TISVGELSRNVLDELAHILTLNTEREGIEEAINTKLKDSFSQDQV
LELVQFRKNNSSLFSRGWHNFSLKLMMELIPELYETSEEQMTILT
RLGKQKSKETSKRTKYIDEKELTEEIYNPVVAKSVRQAIKIINEA
TKKYGIFDNIVIEMARENNEEDAKKDYIKRQKANQDEKNAAMEKA
AFQYNGKKELPDNIFHGHKELATKIRLWHQQGEKCLYTGKNIPIS
DLIHNQYKYEIDHILPLSLSFDDSLSNKVLVLATANQEKGQRTPF
QALDSMDDGWSYREFKSYVKESKLLGNKKKEYLLTEEDISKIEVK
QKFIERNLVDTRYSSRVVLNALQDFYKAHKFDTTISVVRGQFTSQ
LRRKWGLEKSRETYHHHAVDALIIAASSQLRLWKKQNNPLIAYKE
GQFVDSETGEILSLSDDEYKELVFKAPYDHFVDTLRSKTFEDSIL
FSYQVDSKYNRKISDATIYATRKAKLDKDKSEETYVLGKIKDIYS
QAGYDAFIKIYNKDKSKFLMYHKDPQTFEKVIEEILRTYPSKELN
DKNKEIPCNPFEKYRQENGPIRKYSKKGNGPEIKCLKYYDNKLGN
YIDITPDGSDNQVVLQSIAPWRTDVYYNHKTGKYEFLGLKYSDLY
FEKGTGKYKISKEKYDNIKKIEGVVETSEFKFTLYKNDLILIKDV
EKGQEQLFRFLSRNNKGKHQVQLKPMNKSDFEKGEKLIDIFGTVP
NSTTQCVKGLNKSNISIFKVKTDVLGKKHIIKKEGDEPKLKF

10. The Cas9 protein of claim 1, wherein the Streptococcus oralis SK313 Cas9 has at least 80% sequence identity to
(SEQ ID NO: 9)
MNGLVLGLDIGIASVGVGILEKNSGKIVHASSRIFPAATADNNVE
RRKNRQARRLHRRKKHRGARLKDLFEYYGLLTDFSKVSINLNPYR
LRVDGLDQQLTNEELFIALKNIVKRRGISYLDDASEDGGTVSSDY
GKAVEENRKLLAEQTPGQIQLERFEKYGQLRGDFTVVENSEKCRL
INVESTSAYKKEAERILRKQQEFNNQITDEFIEDYLKILTGKRKY
YHGPGNEKSRTDYGRFRTDGATLDNIFGILIGKCTFYPNEYRASK
ASYTAQEFNLLNDLNNLTVPTETKKLSEEQKKTIIEYAKSAKTLG
ASTLLKYIAKMIDASVDQIRGYRVDVNNKPEMHTFEVYRKMQSLE
TISVGELSRNILDELAHILTLNTEREGIEEAINTKLRDSFSQDQV
LELVQFRKNNSSLFSKGWHNFSLKLMMELIPELYETSEEQMTILT
RLGKQKSKETSKRTKYIDEKEVTEEIYNPVVAKSVRQAIKIINEA
TKKHGIFDNIVIEMARENNEEDAKKDYIKRQKANQDEKYAAMEKA
AFQYNGKKELPDNIFHGHKELATKIRLWHQQGEKCLYTGKSIPIS
DLIHNQYKYEIDHILPLSLSFDDSLSNKVLVLATANQEKGQRTPF
QALDSMDDAWSYREFKSYVKESKLLGNKKKEYLLTEEDISKIEVK
QKFIERNLVDTRYSSRVVLNALQDFYKNHNFDTTISVVRGQFTSQ
LRRKWGLEKSRETYHHHAVDALIIAASSQLRLWKKQNNPLIAYKE
GQFVDSQTGEIISLTDDEYKELVFKAPYDHFVDTLSSKTFEDSIL
FSYQVDSKFNRKISDATIYATRKAKLDKEKKEYTYTLGKIKDIYS
LGTKTPSKTGFYKFLDLYNKDKSQFLMFQKDRKTWDEVIEKIMEQ
YRPFKEYDKAGKLVDFNPFEKYRQENGPIRKYSKKGNGPEIKSLK
YYDILLGKHKNITPEGSRNTVALLSLNPWRTDVYYNMETKKYEFL
GLKYADLPFEEGGAYGISTETYNELREKEGIGKNSEFKFTLYKND
LILIKDTETNCQQFFRFWSRTGKDNPKSFEKHKIELKPYEKAKFE
KGEELEVLGKVPPSSNQFQKNMQIENLSIYKVKTDVLGNKHFIKK
EGDKPKLKF

11. The Cas9 protein of claim 1, wherein the Staphylococcus warneri strain 691 Cas9 has at least 80% sequence identity to
(SEQ ID NO: 10)
MKEKYILGLDLGITSVGYGIINFETKKIIDAGVRLFPEANVDNNE
GRRSKRGSRRLKRRRIHRLDRVKSLLTEYNLINREQIPTSNNPYQ
IRVKGLSEILSKDELAIALLHLAKRRGIHNINVSSEDEDASNELS
TKEQINRNNKLLKNKYVCEVQLQRLKEGQIRGEKNRFKTTDILKE
IDQLLKVQKDYHNLDIDFINQYKEIVETRREYFEGPGQGSPFGWN
GDLKKWYEMLMGHCTYFPQELRSVKYAYSADLFNALNDLNNLIIQ
RNDSEKLEYHEKYHIIENVFKQKKKPTLKQIAKEIGVNPEDIKGY
RITKSGTPQFTEFKLYHDLKSIVFDKSILENEAILDQIAEILTIY
QDEESIKEELNKLPEILNEQDKAEIAKLTGYNVTHRLSLKCIHLI
NEELWQTSRNQMEIFNYLNIKPNKVDLSEQNKIPKDLVDEFILSP
VVKRTFIQSINVINKVIEKYGIPEDIIIELARENNSDDRKKFINN
LQKKNEATRKRINEIIGQTGNQNGKRIVEKIRLHDQQEGKCLYSL
ESIPLMDLLNNPQNYEVDHIIPRSVAFDNSIHNKVLVKQIENSKK
GNRTPYQYLNSSDANLSYNQFKQHILNLSKSKDRISKKKKDYLLE
ERDINKFEVQKEFINRNLVDTRYATRELTSYLKAYFSANNMDVKV
KTINGSFTNHLRKVWRFDKYRNHSYKHHAEDALIIANADFLFKEN
KKLQNANKILEKPTIENDTQKVTVEKEEDYNNMFETPKLVEDIKQ
YRDYKFSHRVDKKPNRQLIKDTLYSTRMKDEHNYIVQTITDIYGK
DNTNLKKQFNKNPEKFLMYQNDPKTFEKLSIIMKQYSDEKNPLAK
YYEETGEYLTKYSKKNNGPIVKKIKLLGNKVGNHLDVTNKYENST
KKLVKLSIKNYRFDVYLTEKGYKFVTIAYLNVFKKDNYYYIPKDL
YQELKAKKKIKDTDQFIASFYKNDLIKLNGDLYKIIGVNSDDRNI
IELDYYDIKYKDYCEINNIKGEPRIKKTIGKKTESIEKLTTDVLG
NLYLHTTEKAPQLIFKRGL

12. The Cas9 protein of claim 1, wherein the Staphylococcus sciuri strain SNUC 2430 Cas9 has at least 80% sequence identity to
(SEQ ID NO: 11)
MKRNYILGLDIGITSVGYGIIDYETRDVIDAGVRLFKEANVENNE
GRRSKRGARRLKRRRRHRLQRVKKLLFDYNLLTDHSELSGINPYE
ARVKGLSQKLSEVEFSAALLHLAKRRGVHNVNEVEEDTGNELSTK
EQISRNSKALEEKYVAELQLERLKTDGEVRGPNNRFKTSDYVKEA
KQLLKVQKAYHQLDQSFIDTYIDLLETRRTYYEGPGEGSPFGWKD
IKEWYEMLMGHCTYFPEELRSVKYAYNADLYNALNDLNNLVIARD
ENEKLEYYEKFQIIENVFKQKKKPTLKQIAKELLVNEEDIKGYRV
TSIGKPEFTNFKIYHDIKGITERKEVLENAELLDQIAEILTIYQS
SEDVQEELANLNSELTQEEIEQISNLKGYTGTHNLSLKAINLILD
ELWHTNDNQMAIFNRLKLVPKKVDLSQQKEIPTTLVDDFILSPVV
KRSFIQSIKVINAIIKKYGLPNDIIIELAREKNSKDAQKMINEMQ
KRNRQMNERIEEIIRTTGKENAKYLIEKIKLHDMQEGKCLYSLES
IPLEDLLNNPFNYEVDHIIPRSVSFDNSFNNKVLVKQEENSKKGN
RTPFQYLSSSDSKISYETFKKHILNLAKGKGRISKTKKEYLLEER
DINRFSVQKDFINRNLVDTRYATRELMNLLRSYFRVNNLDVKVKS
INGGFTSFLRRKWKFKKERNKGYKHHAEDALIIANADFIFKEWKK
LDKAKKVMENQTVEEKQAESMPEIETEQEYKEIFITPHQIQHIKG
FKDYKYSHRVDKKPNRELINDTLYSTRKDDKGNTLIVNNLNGLYD
KDNDKLKKLMNKSPEKLLMYHHDPQTYQKLKLIMEQYGDEKNPLY
KYYEETGNYLTKYSKKDNGPVIKKIKYYGKNLKAHLDITDDYPNS
RNKVVKLSVKPYRFDVYLDNDIYKFVTVKNLDVIKKEDYYEVNSK
CYKEAKKLKKISDQAEFIASFYNNDLIKINGELYRVIGVNNDLLN
RIEVNMINITYREYLENMNDKRPPRIIKTIASKTQSIKKYSTDIL
GNLYEVKSKQKPQMIMKG

13. The Cas9 protein of claim 1, wherein the Streptococcus gallolyticus strain AM24-4 Cas9 has at least 80% sequence identity to
(SEQ ID NO: 12)
MTNGKILGLDIGIASVGVGIIEAKTGKVVHANSRLFSAANAENNT
ERRGFRGSRRLNRRKKHRVKRVRDLFEKHEIVTDFRNLNLSPYEL
RVKGLTEQLTNEELFAALRTISKRRGISYLDDAEDDSTGSTDYAK
SIDENRRLLKNKTPGQIQLERLEKYGQLRGNFTVYDENGEAHRLI
NVESTSDYEKEARKILETQADYNKKITAEFIDDYVEILTQKRKYY
HGPGNEKSRTDYGRFRTDGTTLENIFGILIGKCNFYPDEYRASKA
SYTAQEYNFLNDLNNLKVPTETGKLPTEQKESLVEFAKNTATLGP
SKLLKEIAKILDCKVDEIKGYREDDKGKPDLHTFEPYRKLKFNLE
SINIDDLSREVIDKLADILTLNTEREGIEDAIKRNLPNQFTEEQI
SEIIKVRKSQSTAFNKGWHSFSAKLMNELIPELYATSDEQMTILT
RLEKFKVNKKSSKNTKTIDEKEVTDEIYNPVVAKSVRQTIKIINA
AVKKYGDFDKIVIEMPRDKNADDEKKFIDKRNKENKKEKDDALKR
AAYLYNSSDKLPDEVFHGNKQLETKIRLWYQQGERCLYSGKPIPI
HDLVHNSNNFEIDHILPLSLSFDDSLANKVLVYDWANQEKGQKTP
YQVIDSMDAAWSFREMKDYVLKQKGLGKKKRDYLLTTENIDKIEV
KKKFIERNLIDTRYASRVVLNSLQSALRELCKDTKVSVIRGQFTS
QLRRKWKIDKSRETYHHHAVDALIIAASSQLKLWEKQDNLMFIDY
GNNQVVDKETGEILSVSDDEYKELVFQPPYQGFVNTISSKGFEDE
ILFSYQVDSKYNRKVSDATIYSTRKAKIGKDKKEETYVLGKIKDI
YSQNGFDTFIKKYNKDKTQFLMYQKDPLTWENVIEVILRDYPTTK
KSEDGKNDVKCNPFEEYRRENGLICKYSKKGKGTPIKSLKYYDKK
LGNCIDITPEESRNKVILQSINPWRADVYFNPETLKYELMGLKYS
DLSFEKGTGKYHISQEKYDAIKEKEGIGKKSEFKFTLYRNDLILI
KDTASGEQEIYRFLSRTMPNVNHYAELKPYDKEKFDGGQELMEVF
GKVANGGQCLKSLNKSNISIYKVRTDVLGNKYFVKKEGDKPKLNF
KNNKK

14. The Cas9 protein of claim 1, wherein the Lactobacillus kullabergensis strain Biut2 Cas9 has at least 80% sequence identity to
(SEQ ID NO: 13)
MKRVNEDYILGLDIGTNSCGWAVTDKKNNLLKLRGKTAIGSHLFE
EGHTAADRRGFRTTRRRLKRRKWRLRLLEEIFAEPMAKVDPGFFV
RLHQSWVSPLDKDRKKYNAIVFPTAKEDQAFYKHYATIYHLRDEL
MTQDRQFDLREIFLAIHHIVKYRGNFLQDTPVKDFEASKIEVGPI
LSHINNAFAEKIVEDQDPIELNVANAADIEDVIRGKDAEKTVYKL
DKVKKIAKLLTDSTAKEEKNVAKQIANAIMGYKTQFETILDKEID
KSDKAQWEFKLSDADADDKLDALLPDLDETDQTVVAEIEKLFSAI
TLSTIVDENKSLSQSMVEKYKKHKKDYKKLKKYINTLQDQTKAKK
LLLAYDLYVNNRHGRLLEAKKTFEDKKKKKALTKDEFYKIVKDNL
DDSDLAHEIQQEIAADNFMPKQRTNSNGVIPFQLHQIELDKIIAN
QGKYYPFLAAENPVEDHRKQAPYKLDELVRFRVPYYVGPMITADE
QEKTSGKSFAWMVRKEDGQITPWNFEQKVDRQESANKFIKRMTIK
DTYLLSEDVLPANSLLYQRFEVLNELNNIRINGSRISVDLKQQIF
NDLFEEKKTVTEKSLTSYLKQNLHLPTVEIKGLADPTKFNSSLAS
YYHLKSLHVFDKELADPQYQKDFEKIIEYSSIFEDKKIFQDKLHA
EFKWLTPEQFKAISTWRLQGWGRLSRKLLVELHDTNGQNIMEQLW
DSQKNFMQIVTEPDFKDAIAKENQNVTRANGVEEILADAYTSPAN
KKAIRQVVKVVADVVKAAGGKKPAQFAIEFTRDPDKNPQLSHIRG
TKLLKAYQETAGELVDQKLTDSLKEAMTSRKLLKDKYFLYFMQAG
RDAYTGQKINIDEVSTNYQIDHILPQSFIKDDSFDNRVLTATPLN
AEKSDDVPYKRFANNYVSDMKMTVGEMWKHWQKAGIINKHKLGNL
LLDPDRLNKFQKSGFINRQLVETSQIIKLVSVILQNKYPDAEIIT
VKAGDNSALRQRLNLYKSRDVNDYHHAIDAYLSIICGNFLYQVYP
KYRPYFVYGKYKKFSQDPDLQKEVIKHFKGFTFMWPLLQKDNSER
KAPEKIKENNSDRIVFYKHPDIFDKLRKAYNYKYMLVSRETTTEN
SGLFDVTIYPRGERDLAKTRKLIPKSNGLDPKIYGGYSGNTDAYM
VIVKIDKGKESIYKVIGVPMRALASLNRAKKQGNYKEELHQVLEP
QIMFDKNGKPKRSVKGFRIIKDHVPFKQVVLDGDKKFMLNSSTYE
INAKQLTLTPETMRIVTDNLKKGEDQDQLLVKAYDEILQKVDQYL
PLFDVNKFRNSLHLGRAKFLDLAVNDKKITLTNILNGLHDNLVTP
DLKNIGIKTPLGKLQVPSGIVLSSEAILIFQSPTGLFEKRVRIADL

15. The Cas9 protein of claim 1, wherein the Streptococcus suis strain LSS83 Cas9 has at least 80% sequence identity to
(SEQ ID NO: 14)
MSNGKILGLDIGIASVGVGVIDAQTGEIIHASSRIFPSANAANNA
ERRTFRGSRRLIRRKKHRIKRLDDLFNDFHINLDGEMSTDNPYVL
RVKGLSQKLTVEELYISIKNIMKRRGISYLDDAESDNEAGRSDYA
KAIERNRQLLTSKTPGEIQLERLEKYGQLRGNFTIIDEEGQSQQI
INVESTSDYVKEVEKILDCQKMYHKFISDEFCDKLIELLREKRKY
YVGPGNEKSRTDYGIYRTDGTTLENLFGILIGKCTFYPDQYRSSR
ASYTAQEFNFLNDLNNLTVPTETKKLSQEQKEFLVNYAKETSVLG
AGKILQQIAKLADCKVEDIRGYRLDNKDKPELHTFETYRAMKGLV
PLVDIGVLSREQLDILADILTLNTDFEGIREALKKQLPNVFDEKQ
VKGLASFRKSKSQLFAKGWHNLSQKIMLEVIPELYATSDEQMTIL
TRLGKFEKSSVAEYPSSINVDEITDEIYNPVVAKSIRQTIKIINA
SIKKWDEFDQIVIEMPRDRNEDEEKKRIADGQKANAKEKADSILR
AAELYCAGKVLPDYVYNGHNQLATKIRLWYQQGERCIYTGQPISI
HDLIHNQNQYEIDHILPLSLTFDDSLSNKVLVLATANQEKAQRTP
YNYLKSATSAWSYREFKDYVTKRKGIGKKKCEYLTFEEDINGFEV
RSKFIQRNLVDTRYASKVILNALQDYFKISGIQTKVSVVRGQFTS
QLRHKWGIEKTRETYHHHAVDALIIAASSQLRLWKKQESPLVVDY
QEGRQVDLETGEILELTDEQYKELVYQPPYQGFVNTISSSAFDNE
ILFSYQVDSKVNRKISDATIYATRNAQLGKDKTEGIYVLGKIKDI
YTQAGYEAFLKRYTKDKTSFLMYHKDLDTWEKVIEIILRDYREYD
EKGKEIGNPFERYRRENGYVKKYSRKGNGTAIKSLKYYDNKLGNH
IDITPENSRNAVVLQSLKPWRTDVYFNKETGKYEFLGIKYSDLSF
EKGTGEYGISQEKYDSIKIAEGVAKKSIFKFTLYKQDLLFIKDIE
NNFGKLLRFTSKNDTSKHYVELKPYDKNKFGTEEPLLPVLGNVAK
SGQCIKGLNKSNISIYKVRTDILGYRHFIKQEGEHPQLKFKK

16. The Cas9 protein of any one of claims 1-15 comprising an amino acid sequence that is at least 85%, at least 90%, at least 92%, at least 95%, at least 96%, at least 97%, at least 98%, or at least 99% identical to SEQ ID Nos: 1, 2, 3, 4, 5, 6, 7, 8, 9, 10, 11, 12, 13 or 14.

17. The Cas9 protein of any one of the preceding claims, further comprising a nuclear localization sequence (NLS) and/or a FLAG, HIS or HA tag, and/or a deaminase.

18. The protein of claim 17, wherein the Streptococcus equinus ATCC 33317 Cas9 has an amino acid sequence at least 80% identical to
(SEQ ID NO: 29)
MPAAKRVKLDGSEVEFSHEYWMRHALTLAKRARDEREVPVGAVLV
LNNRVIGEGWNRAIGLHDPTAHAEIMALRQGGLVMQNYRLYDATL
YVTFEPCVMCAGAMIHSRIGRVVFGVRNAKTGAAGSLMDVLHHPG
MNHRVEITEGILADECAALLCRFFRMPRRVFNAQKKAQSSTDGSS
GSETPGTSESATPESSGPKKKRKVGTMEKSYSIGLAIGTNSVGWS
VITDDYKVPAKKMRVLGNTDKKYIKKNLLGALLFDSGETAEATRL
KRTARRRYTRRKNRLRYLQEIFAKEMAKVDESFFQRLDESFLTDD
DKTFDSHPIFGNKAEEDAYHQKFPTIYHLRKHLADSQEKADLRLI
YLALAHMIKFRGHFLYDNFNDDNFDWRNIDIQKRYEEFIETYDST
LGESYLADISVDAASILEEKVSKTERLENLLKYYPTEKKTTFFGN
LIKLILGQQAKFKAIFNLEDEISLQFATPTYDEDLEELLGKIDNG
DSYSELFVAAQNLYNTILLASFLKTDNKSAKAPLSTSMIERYENH
KKDLAKLKDFVKKNCPDQYHDIFRDKSKNGYAGYIDNGVSQNDFY
TFIGKCLEESLKKDKGAQYFLDKIDRDDFLRKQRTFDNGAFPYQI
HLQEMHAILRRQGDYYPFLKENQDKIEKILTFRIPYYVGPLARKD
SRFAWANYRSDEAITPWNFDEVVDKEKSAEKFITRMTLNDLYLPE
EKVLPKHSLIYETFTVYNELTNIKYVNDQGNAIHFDSELKEKIFN
QLFKENRKVSKKTLIDFLNNSEGIYTDKLVGIDEEVKYLNASLGT
YHDLKKILESFMDDEINEKIIEDIIQTLTLFEDIEMKRQRLQKYD
DIFTPKQLKELARRNYTGWGRLSYKLINGIRNKENNKTILDYLKN
GNRNFMQLINDDRLSFKQIIIDARKIEKLDNIESVVYNLPGSPAI
KKGILQSIKIVDELVKVMGHNPDNIVIEMARENQTTNQGKNRSQQ
RLKRLQDSMSNFKDSSISLKDVDNSDLQNDRLFLYYIQNGKDMYT
GEELDIDHLSDYDIDHIIPQSFIKDNSIDNRVLTSSAKNRGKSDD
VPGRDVVLKMKPFWKKLYDVKLISKRKFDNLTKSEHGGLTESDKA
GFIKRQLVETRQITKYVAQILDGRFNTKRDDNNKVIRDVKVITLK
SSLVSQFRKDFGFYKVREINDYHHAHDAYLNAVVGTAILKKYPKL
APEFVYGEYKKCDVRKLIAKSGDKSEIGKATAKYFFYSNLMNFFK
RVIRYSNGMIVVRPVIEYSKDTGEIAWDKEKDFKTVCKVLSCPQV
NIVKKVEKQSHGLDRGKPKGFYNANPSPKPKKGSKVNLVPIKANL
NPKNYGGYAGISNSYAVLVDATIEKGAKKKLTRIQEFQGISIIDR
EKYEKNKVEFLKGLGYKEIYSIITLPKYSLFELADGSRRMLASIL
STNNKRGEIHKGNELVLPAKYIPLLYHANRIHNTFETGHREYVEK
HIAEFKEIAEIILEFNNKYVNAKKNSSIIEKALESFDSFSLDEIC
DSFVGKLKKNNTKKNSGLFELVSLGSASDFEFLETKVPRYRDYTP
SSLLNATLIHQSITGLYETRIDLSKLGEEKRPAATKKAGQAKKKK
GSYPYDVPDYAYPYDVPDYAYPYDVPDYA.

19. The protein of claim 17, wherein the Enterococcus hirae strain F1129E Cas9 has an amino acid sequence at least 80% identical to
(SEQ ID NO: 30)
MPAAKRVKLDGSEVEFSHEYWMRHALTLAKRARDEREVPVGAVLV
LNNRVIGEGWNRAIGLHDPTAHAEIMALRQGGLVMQNYRLYDATL
YVTFEPCVMCAGAMIHSRIGRVVFGVRNAKTGAAGSLMDVLHHPG
MNHRVEITEGILADECAALLCRFFRMPRRVFNAQKKAQSSTDGSS
GSETPGTSESATPESSGPKKKRKVGTMTKDYTIGLAIGTNSVGWA
VLTDDYQLMKRKMSVHGNTEKKKIKKNFWGARLFDEGQTAEFRRT
KRTNRRRLARRKYRLSKLQDLFAEELCKQDDCFFVRLEESFLVPE
EKQYKPASIFPTLEEEKEYYQKYPTIYHLRQKLVDSTEKEDLRLV
YLALAHLLKYRGHFLFEGDLDTENTSIEESFRVFLEQYSKQSDQP
LIVHQPVLTILTDKLSKTKKVEEILKYYPTEKINSFFAQCLKLIV
GNQANFKRIFDLEAEVKLQFSKETYEEDLESLLEKIGDEYLDIFL
QAKKVHDAILLSEIISSTVKHTKAKLSSGMVERYERHKADLAKFK
QFVKENVPQKSTAFFKDTTKNGYAGYIKGKTTQEEFYKFVKKELS
GVVGSEPFLEKIDQETFLLKQRTYTNGVIPHQVHLIELKAIIDQQ
KQHYPFLEEAGPKIIALFKFRIPYYVGPLAKEQEASSFAWIERKT
AEKIHPWNFSEVVDIEKSAMRFIQRMTKQDTYLPTEKVLPKNSLL
YQKYMIFNELTKVSYKDERGVKQYFSGDEKQQIFKQLFQKERGKI
TVKKLQNFLYTHYHIENAQIFGIEKAFNASYSTYHDFMKLAKTNQ
KAMQEWLEQPEMEPIFEDIVKILTIFEDRQMIKHQLSKYQEVFGE
KLLKEFARKHYTGWGRFSAKLIHGIRDRKTNKTILDYLINDDDVP
ANRNRNLMQLINDEHLSFKEEIAKATVFSKHKSLVDVIQDLPGSP
AIKKGIWQSLKIVEELIAIIGYKPKNIVIEMARENQKTHRTKPRL
KALENGLKQIGSTLLKEQPTDNKALQKERLYLYYLQNGRDMYTGE
PLEIENLHQYEVDHIIPRSFIVDNSIDNKVLVARKQNQKKRDDVP
KKQIVNEQRIFWNQLKEAKLISPKKYAYLTKIELTPEDKARFIQR
QLVETRQITKHVANILHQSFHQEEEGTDCDGVQIITLKATLTSQF
RQTFGLYKVREINPHHHAHDAYLNGFIANVLLKRYPKLAPEFVYG
KYVKYSLARENKATAKKEFYSNILKFFESDEPFCDENGEIYWEKS
HHLPRIKKVLSSHQVNVVKKVEQQKGGFYKETVNSKEKPDKLIER
KNNWDVTKYGGFGSPVIAYAIAFVYAKGKTQKKTRAIEGITIMEQ
AAFEKDPTTFLKEKGFPQVTEFIKLPKYTLFEFDNGRRRFLASHK
ESQKGNPFILSDQLVTLLYHAQHYDKITYQESFDYVNTHLSDFSA
ILTEVLAFAEKYTLADKNIERIQELYEENKYGEISMIAQSFLQLL
QFNAIGAPADFKFFGVTIPRKRYTSLTEIWDATIIYQSVTGLYET
RIRMGDLWAGEQKRPAATKKAGQAKKKKGSYPYDVPDYAYPYDVP
DYAYPYDVPDYA

20. The protein of claim 17, wherein the Streptococcus equinus strain AG46 Cas9 has an amino acid sequence at least 80% identical to
(SEQ ID NO: 31)
MPAAKRVKLDGSEVEFSHEYWMRHALTLAKRARDEREVPVGAVLV
LNNRVIGEGWNRAIGLHDPTAHAEIMALRQGGLVMQNYRLYDATL
YVTFEPCVMCAGAMIHSRIGRVVFGVRNAKTGAAGSLMDVLHHPG
MNHRVEITEGILADECAALLCRFFRMPRRVFNAQKKAQSSTDGSS
GSETPGTSESATPESSGPKKKRKVGTMTNGKILGLAIGVASVGVG
IIEAKTGKVIHANSRLFSAANAENNAERRGFRGARRLTRRKKHRV
KRVRDLFEKYDISTDFRNLNLNPYELRVKGLSEQLTNEELFAALR
TIAKRRGISYLDDAEDDSTGSSDYAKSIDENRRLLKSMTPGQIQL
ERLEKYGQLRGNFTVYDENGEAHRLINVESTSDYKNEARKILETQ
SNYNKQITDEFIEDYIEILTQKRKYYHGPGNEKSRTDYGRFRTDG
TTLENIFGILIGKCSFYPEEYRASKASYTAQEFNFLNDLNNLKVP
TETGKLSTEQKEYLVDFAKNSKTLGASKLLKEIAKLVDGDVKDIS
GYREDSKGKPDLHTFEPYRKLKFNLTTVDIDNLSRDILDKLANIL
TLNTEREGIEDAINRNLPEQFTKEQISEIVQIRKSQSSAFNKGWH
SFSAKLMNELIPELYATSEEQMTILTRLEKFKATKKSSKNTKTID
EKEITDEIYNPVVAKSVRQTIKIINAAVKKYGDFDKIVIEMPRDK
NADDEKKFIDKKNKENKKEKDDSLKRAAYLYNGTDKLPDDVFHGN
KQLATKIRLWYQQGERCLYSGKPILIQDLVHNSNNFEIDHILPLS
LSFDDSLANKVLVYAWTNQEKGQKTPYQVIDSMDAAWSFREMKDY
VLKQKGIGKKKREYLLTTENIDKIEVKKKFIERNLVDTRYASRVV
LNSLQTALKELGKDTKVSVVRGQFTSQLRRKWKIDKSRETYHHHA
VDALIIAASSQLKLWQKHENLMFENYGENQVVNKETGEILSISDD
EYKELVFQPPYQGFVNTISSKAFEDEILFSYQVDSKFNRKVSDAT
IYSTRKAKLGKDKKEETYVLGKIKDIYSQDGFDTFIKRYKKDKTQ
FLMYQKDPLTWENVIEVILRDYPTTKKSEDGKNDVKCNPFEEYRR
ENGLICKYSKKGKGTPIKSLKYYDKKLGNHISITPKESKNDVVLQ
SLNPWRADLYFNPDTLKYELMGLKYSDLSFEKGTGKYHISQEKYD
EIKEKEGIGQNSEFKFTLYRNDLILIKDTESGEQEIYRFLSRTMP
NVKHYVELKPYDKEKFNGGQELIKSLGEADKVGRCLKGLSKPGIS
IYKVRTDVLGNKFFVKKEGDKPKLDFKNNKKKRPAATKKAGQAKK
KKGSYPYDVPDYAYPYDVPDYAYPYDVPDYA.

21. The protein of claim 17, wherein the Streptococcus intermedius B196 strain G1552 Cas9 has an amino acid sequence at least 80% identical to
(SEQ ID NO: 32)
MPAAKRVKLDGSEVEFSHEYWMRHALTLAKRARDEREVPVGAVLV
LNNRVIGEGWNRAIGLHDPTAHAEIMALRQGGLVMQNYRLYDATL
YVTFEPCVMCAGAMIHSRIGRVVFGVRNAKTGAAGSLMDVLHHPG
MNHRVEITEGILADECAALLCRFFRMPRRVFNAQKKAQSSTDGSS
GSETPGTSESATPESSGPKKKRKVGTMNGLVLGLAIGIASVGVGI
LNKETGEIIHVNSRIFPAATADSNVERRGFRQGRRLGRRKKHRSA
RLNDLFEEFGFITDFSAVPLNLNPYALRVKGLSEELTNEELFIAL
KNIIKRRGISYLDDASEDGETASNEYGKAVEENRKLLADKTPGQI
QLERFEKYGQVRGDFTVVENGENHRLINVESTSAYKKEAERILRR
QQEFNVRISDEFIEAYLTILTGKRKYYHGPGNEKSRTDYGRFRTD
GTTLDNIFGILIGKCTFYPDEYRAAKASYTAQEFNLLNDLNNLTV
PTETKKLRPEQKRQIVEYARTAKTLGTPTLLKYIAKLVDGSIDDI
KGYRIDKSDKPEMHTFDAYRKMRTLELVDVDILSRETLDDLAYIL
TLNTESEGILEALNSKMPGTFTKEQIDELIQFRKKNSAVFGKGWH
NFSLKLMNELISELYETSEEQMTILTRLGKQRSREISKRTKYIDE
KELTDEIYNPVVAKSVRQAIKIINLATKKYGIFDNIVIEMARESN
EDDEKKAIQNVQKANEDEKKAAMEKAADLYNGKKELPDSIFHGHK
ELATKIRLWHQQGERCLYTGKNISIHDLIHNPHQYEIDHILPLSL
SFDDGLANKVLVLATANQEKGQRTPFQALDSMDDAWSYIEFKQYV
RNSKSLSNKKKDYLLTEEDISKIEVKQKFIERNLVDTRYSSRVVL
NTLQEFYKTNDFDTKISVVRGQFTSQLRRKWKIEKSRDTYHHHAV
DALIIAASSQLRLWKKQNNPLISYREGQLVDPETGEILSLTDDEY
KELVFRPPYDYFVDTLKSKSFEDSILFSYQVDSKYNRKISDATIY
GTRKAQLGKDKQEETYVLGKIKDIYSQKGYEDFIKRYKKDTTQFL
MYHKDPQTFAKVIEEILKTYPDKELNEKGKEIPCNPFEKYRQENG
PIRKYSKKGKGPEIKSLKYYDNKLGNHIDITPVNSQNQVVLQSLK
PWRTDVYFNPQTSKYELMGLKYSDLRFEKGSGSYGISPEKYNKVK
AKEGVDEDSEFKFTLYKNDLILIKDTETGEQQLFRYGSRNDTSKH
YVELKPYEKAKFEGNQQLMNLLGTVAKGGQCLKGINKPNLSIYKV
KTDVLGNKHFIKKEGDQPQLNFKKKIKRPAATKKAGQAKKKKGSY
PYDVPDYAYPYDVPDYAYPYDVPDYA.

22. The protein of claim 17, wherein the Staphylococcus simulans strain 19 Cas9 has an amino acid sequence at least 80% identical to
(SEQ ID NO: 33)
MPAAKRVKLDGSEVEFSHEYWMRHALTLAKRARDEREVPVGAVLV
LNNRVIGEGWNRAIGLHDPTAHAEIMALRQGGLVMQNYRLYDATL
YVTFEPCVMCAGAMIHSRIGRVVFGVRNAKTGAAGSLMDVLHHPG
MNHRVEITEGILADECAALLCRFFRMPRRVFNAQKKAQSSTDGSS
GSETPGTSESATPESSGPKKKRKVGTMNNSYILGLAIGITSVGYG
IIEYETRDVIDAGVRLFKEANVENNEGRRSKRGARRLKRRRRHRL
QRVKKMLFDYKLLNEDSEISGINPYEARVKGLSEKLSDEEFSAAL
LHLAKRRGVHNVSDVEEDTGNELSTKEQIARNNKALEDKYVAELQ
LERLKEQGEVRGAANRFKTSDYIKEAKQLLKTQSDYHKIDETFIE
TYISLLETRRTYYEGPGEGSPFGWKDIKEWYEMLMGHCTYFPEEL
RSVKYAYNADLYNALNDLNNLVIARDENEKLEYYEKFQIIENVFK
QKKKPTLKQIAKEILVNEEDIKGYRVTSTGKPEFTNFKIYHDIKG
ITSRKEILENADLLNQIAEILTIYQSAEDIQEELAELEPKLTQEE
IEQISNLTGYTGTHRLSLKAINLILDELWNTSDNQMTIFNRLKLV
PKKVDLSQQKEIPTSLVDDFILSPVVKRSFIQSIKVINAIIKKFG
LPKDIIIELAREKNSKEAQKFINEMQKRNRQTNERIEKIIKETGK
EKAKFLIEKIKLHDMQEGKCLYSLESIPLEDLLNNPYHYEVDHII
PRSVSFDNSFNNKVLVKQEENSKKGNRTPFQYLSSSDAKISYETF
KKHILNLSKGKGRVSKKKKEYLLEERDINRFSVQKDFINRNLVDT
RYATRELMNLLRSYFRVNDLDVKVKSINGGFTSFLRTKWKFKKER
NQGYKHHAEDALVIANADFIFKEWKKLDTTNKVMENQTVEEQQAE
NMPGIETDDEYKEIFVIPRQIQSIKDFKDYKYSHRVDKKPNRELV
NDTLYSTRKDDKGNTLIINNIKGLYDKDNDKLKNLIKKSPEKLLM
YHHDPQTYQKLKTIMEQYSNEKNPLYKYHEETGNYLTKYSKKDNG
PIIKKVKYYGKKLNAHLDITNDYSNSQNKIVKLSLKPYRFDVYLD
NGGYKFVTVKNLDVIKKEGFFKIDSNAYEKAKSEKKIDENAVFIA
SFYNNDLIKIDGELYRIVGVNNDTRNVVELNMIPITYKEYLENIN
DKRTPRILKTISQKTYSIEKYSTDILGNLYKVKSKKKPQMIMKGK
RPAATKKAGQAKKKKGSYPYDVPDYAYPYDVPDYAYPYDVPDYA

23. The protein of claim 17, wherein the Streptococcus sanguinis SK330 Cas9 has an amino acid sequence at least 80% identical to
(SEQ ID NO: 34)
MPAAKRVKLDGSEVEFSHEYWMRHALTLAKRARDEREVPVGAVLV
LNNRVIGEGWNRAIGLHDPTAHAEIMALRQGGLVMQNYRLYDATL
YVTFEPCVMCAGAMIHSRIGRVVFGVRNAKTGAAGSLMDVLHHPG
MNHRVEITEGILADECAALLCRFFRMPRRVFNAQKKAQSSTDGSS
GSETPGTSESATPESSGPKKKRKVGTMENKNYSIGLAIGTNSVGW
AVITDDYKVPSKKMKVFGNTDKHFIKKNLIGALLFDEGATAEDRR
LKRTARRRYTRRKNRLRYLQEIFSEEISKLDSSFFHRLDDSFLVP
KDKRGSKYPIFATLEEEKEYHKKFPTIYHLRKHLADSKEKTDLRL
IYLALAHMIKYRGHFLYEESFDIKNNDIQKIFNEFISIYDNTFEG
SSLSGQNAQVEAIFTDKISKSAKRERVLKLFSDEKSTSLFSEFLK
LIVGNQADFKKHFDLEEKAPLQFSKDTYDEDLENLLGQIGDGFTD
LFLVAKKLYDAILLSGILTVTDPSTKAPLSASMIERYESHQKDLA
ALKQFIKNNLPKRYNEVESDQSKDGYAGYIDGKTTQEAFYKYIKN
LLSKFEGADYFLDKIEREDFLRKQRTFDNGSIPHQIHLQEMNAIL
RRQGEHYPFLKENREKIEKILTFRIPYYVGPLARGNRDFAWLTRN
SDQAIRPWNFEEIVDKASSAEEFINKMTNYDLYLPEEKVLPKHSL
LYETFAVYNELTKVKFIAEGLRDYQFLDSGQKKQIVNQLFKEKRK
VTEKDIIHYLHNVDGYDGIELKGIEKQFNASLSTYHDLLKIIKDK
EFMDDPKNEEILENIVHTLTIFEDREMIKQRLAQYDSIFDEKVIK
ALTRRHYTGWGKLSAKLINGICDKQTGDTILDYLIDDGKINRNFM
QLINDDGLSFKEIIQKAQVVGKTDDVKQVVQELPGSPAIKKGILQ
SIKIVDELVKVMGYAPESIVIEMARENQTTARGKKNSQQRYKRIE
DSLKNLAPGLDSNILKENPTDNIQLQNDRLFLYYLQNGKDMYTGK
PLDIDQLSSYDIDHIIPQAFIKDDSIDNRVLTSSKDNRGKSDNVP
SLEVVQKRKAFWQQLLDSKLISERKFNNLTKAERGGLDERDKVGF
IRRQLVETRQITKHVAQILDASFNTEVNEKNQKIRTVKIITLKSN
LVSNFRKEFELYKVREINDYHHAHDAYLNAVVAKAILKKYPKLEP
EFVYGDYQKYDLKRYISRFKPSKEIEKATEKYFFYSNLLNFFKEE
VHYADGIIVKRENIEYSKDTGEIAWNKEKDFATIKKVLSYPQVNI
VKKTEIQTHGLDRGKPKGLFNSNPSPKPSEDSKENLVPIKQGLDP
RKYGGYAGISNSYAVLVKAIVEKGAKKQQKTILEFQGISILDKIN
FENNKENYLLKKRYIEILSTITLPKYSLFEFPDGTRRRLASILST
NNKRGEIHKGNELVLPGKYTTLLYHAKNINKKLEPEHLEYVEKHR
NDFAKLLECVLNFNDKYVGALKNGERIRQAFTDWETVDIEKLCFS
FIGPENSKNAGLFELTSQGSASDFEFLGVKIPRYRDYAPSSLLKA
TLIHQSITGLYETRIDLSKLGEDKRPAATKKAGQAKKKKGSYPYD
VPDYAYPYDVPDYAYPYDVPDYA

24. The protein of claim 17, wherein the Streptococcus sp. C150 Cas9 has an amino acid sequence at least 80% identical to
(SEQ ID NO: 35)
MPAAKRVKLDGSEVEFSHEYWMRHALTLAKRARDEREVPVGAVLV
LNNRVIGEGWNRAIGLHDPTAHAEIMALRQGGLVMQNYRLYDATL
YVTFEPCVMCAGAMIHSRIGRVVFGVRNAKTGAAGSLMDVLHHPG
MNHRVEITEGILADECAALLCRFFRMPRRVFNAQKKAQSSTDGSS
GSETPGTSESATPESSGPKKKRKVGTMSDLVLGLAIGIGSVGVGI
LNKVTGEIIHKNSRIFPAAQAENNLERRTNRQGRRLTRRKKHRRV
RLNHLFEESGLITDFTKVSINLNPYQLRVKGLTAELSNEELFIAL
KNMVKHRGISYLDDASDDGNSSVGDYAQIVKENSKQLETKTPGQI
QLERYQKYGQLRGDFTVEEDGRKHRLINVFPTSAYHAEALRILQT
QQEFNPQITDEFINSYLEILTGKRKYYHGPGNEKSRTDYGKYTTK
KDAQGQYITLNNIFGILIGKCTFYPEEYRAAKASYTAQEFNLLND
LNNLTVPTETKKLSEEQKYQIITYVKNEKAMGPAKLFKYIAKLLS
CDVADIKGYRIDKSDKAEIHTFEAYRKMKTLETLDVKKMAREELD
KLAYVLTLNTEREGIQEALDHEFADGTFSQEQVDELVQFRKANSS
IFGKGWHSFSVKLMMELIPELYATSEEQMTILTRLGKQKTTSSSN
KTKYIDEKQLTEEIYNPVVAKSVRQAIKIVNAAIKKYGDFDNIVI
EMARETNEDDEKKAIQKIQKANKAEKDAAMRKAANQYNGKAELPH
SVFHGHKQLATKIRLWHQQGERCLYTGKTISIHDLINNPNQFEID
HILPLSITFDDSLANKVLVYATANQEKGQRTPYQALDSMDDAWSF
RELKAFVRESKALSNKKKEYLLTEEDISKFDVRKKFIERNLVDTR
YASRVVLNALQEHFRTHKIDTKVSVVRGQFTSQLRRHWGIEKTRD
TYHHHAVDALIIAASSQLNLWKKQKNTLVSYSEDQLLDIETGELI
SDDEYKESVFKAPYQHFVDTLKSKEFEDSILFSYQVDSKFNRKIS
DATIYATRKAKLDKEKKEYTYTLGKIKDIYALGTKTPSKTGFYKF
LDLYKKDKSQFLMYQKDRRTWDEVIEKILEQYRPFKEKDKNGKEV
DENPFEKYRIENGPIRKYSRKGNGPEIKSLKYYDNLLGRFVDITP
SESKNPVALLSLNPWRTDVYYNTETRKYEFLGLKYADLCFEKGGS
YGISKVKYNKIREKEGIGKNSEFKFTLYKNDLILIKDTETNRQQI
FRFWSRTGKDNPKSFEKHKLELKPYEKTRFEKGEELKVLGKVPPS
SNRLQKNMQIENLSIYKVRTDVLGNQHIIKNEGDKPKLDFKRPAA
TKKAGQAKKKKGSYPYDVPDYAYPYDVPDYAYPYDVPDYA

25. The protein of claim 17, wherein the Streptococcus oralis subsp. oralis strain RH_1735_08 Cas9 has an amino acid sequence at least 80% identical to
(SEQ ID NO: 36)
MPAAKRVKLDGSEVEFSHEYWMRHALTLAKRARDEREVPVGAVLV
LNNRVIGEGWNRAIGLHDPTAHAEIMALRQGGLVMQNYRLYDATL
YVTFEPCVMCAGAMIHSRIGRVVFGVRNAKTGAAGSLMDVLHHPG
MNHRVEITEGILADECAALLCRFFRMPRRVFNAQKKAQSSTDGSS
GSETPGTSESATPESSGPKKKRKVGTMNGLVLGLAIGIASVGVGI
LEKNSGKIIHANSRIFPAATADNNVERRKNRQARRLHRRKKHRGV
RLQDIFEDYGLLTDFSKVSINLNPYRLRVDGLDQQLTNEELFIAL
KNIVKRRGISYLDDASEDGGTVSSDYGKAVEENRKLLAEKTPGQI
QLERFEKYGQVRGDFTVVENGEKRRLINVESTSAYRKEAERILRK
QQEFNSKITDEFIEDCLKILTGKRKYYHGPGNEKSRTDYGRFRTD
GTTLDNIFGILIGKCTFYPNEYRASKASHTAQEFNLLNDLNNLTV
PTETKKLSEEQKKVIVEYAKEAKTLGASTLLKYIAKMIDASVDQI
SGYRVDVNNKPEMHTFEVYRKMQSLETISVGELSRNVLDELAHIL
TLNTEREGIEEAINTKLKDSFSQDQVLELVQFRKNNSSLFSRGWH
NFSLKLMMELIPELYETSEEQMTILTRLGKQKSKETSKRTKYIDE
KELTEEIYNPVVAKSVRQAIKIINEATKKYGIFDNIVIEMARENN
EEDAKKDYIKRQKANQDEKNAAMEKAAFQYNGKKELPDNIFHGHK
ELATKIRLWHQQGEKCLYTGKNIPISDLIHNQYKYEIDHILPLSL
SFDDSLSNKVLVLATANQEKGQRTPFQALDSMDDGWSYREFKSYV
KESKLLGNKKKEYLLTEEDISKIEVKQKFIERNLVDTRYSSRVVL
NALQDFYKAHKFDTTISVVRGQFTSQLRRKWGLEKSRETYHHHAV
DALIIAASSQLRLWKKQNNPLIAYKEGQFVDSETGEILSLSDDEY
KELVFKAPYDHFVDTLRSKTFEDSILFSYQVDSKYNRKISDATIY
ATRKAKLDKDKSEETYVLGKIKDIYSQAGYDAFIKIYNKDKSKFL
MYHKDPQTFEKVIEEILRTYPSKELNDKNKEIPCNPFEKYRQENG
PIRKYSKKGNGPEIKCLKYYDNKLGNYIDITPDGSDNQVVLQSIA
PWRTDVYYNHKTGKYEFLGLKYSDLYFEKGTGKYKISKEKYDNIK
KIEGVVETSEFKFTLYKNDLILIKDVEKGQEQLFRFLSRNNKGKH
QVQLKPMNKSDFEKGEKLIDIFGTVPNSTTQCVKGLNKSNISIFK
VKTDVLGKKHIIKKEGDEPKLKFKRPAATKKAGQAKKKKGSYPYD
VPDYAYPYDVPDYAYPYDVPDYA.

26. The protein of claim 17, wherein the Streptococcus oralis SK313 Cas9 has an amino acid sequence at least 80% identical to
(SEQ ID NO: 37)
MPAAKRVKLDGSEVEFSHEYWMRHALTLAKRARDEREVPVGAVLV
LNNRVIGEGWNRAIGLHDPTAHAEIMALRQGGLVMQNYRLYDATL
YVTFEPCVMCAGAMIHSRIGRVVFGVRNAKTGAAGSLMDVLHHPG
MNHRVEITEGILADECAALLCRFFRMPRRVFNAQKKAQSSTDGSS
GSETPGTSESATPESSGPKKKRKVGTMNGLVLGLAIGIASVGVGI
LEKNSGKIIHANSRIFPAATADNNVERRKNRQARRLHRRKKHRGV
RLQDIFEDYGLLTDFSKVSINLNPYRLRVDGLDQQLTNEELFIAL
KNIVKRRGISYLDDASEDGGTVSSDYGKAVEENRKLLAEKTPGQI
QLERFEKYGQVRGDFTVVENGEKRRLINVESTSAYRKEAERILRK
QQEFNSKITDEFIEDCLKILTGKRKYYHGPGNEKSRTDYGRFRTD
GTTLDNIFGILIGKCTFYPNEYRASKASHTAQEFNLLNDLNNLTV
PTETKKLSEEQKKVIVEYAKEAKTLGASTLLKYIAKMIDASVDQI
SGYRVDVNNKPEMHTFEVYRKMQSLETISVGELSRNVLDELAHIL
TLNTEREGIEEAINTKLKDSFSQDQVLELVQFRKNNSSLFSRGWH
NFSLKLMMELIPELYETSEEQMTILTRLGKQKSKETSKRTKYIDE
KELTEEIYNPVVAKSVRQAIKIINEATKKYGIFDNIVIEMARENN
EEDAKKDYIKRQKANQDEKNAAMEKAAFQYNGKKELPDNIFHGHK
ELATKIRLWHQQGEKCLYTGKNIPISDLIHNQYKYEIDHILPLSL
SFDDSLSNKVLVLATANQEKGQRTPFQALDSMDDGWSYREFKSYV
KESKLLGNKKKEYLLTEEDISKIEVKQKFIERNLVDTRYSSRVVL
NALQDFYKAHKFDTTISVVRGQFTSQLRRKWGLEKSRETYHHHAV
DALIIAASSQLRLWKKQNNPLIAYKEGQFVDSETGEILSLSDDEY
KELVFKAPYDHFVDTLRSKTFEDSILFSYQVDSKYNRKISDATIY
ATRKAKLDKDKSEETYVLGKIKDIYSQAGYDAFIKIYNKDKSKFL
MYHKDPQTFEKVIEEILRTYPSKELNDKNKEIPCNPFEKYRQENG
PIRKYSKKGNGPEIKCLKYYDNKLGNYIDITPDGSDNQVVLQSIA
PWRTDVYYNHKTGKYEFLGLKYSDLYFEKGTGKYKISKEKYDNIK
KIEGVVETSEFKFTLYKNDLILIKDVEKGQEQLFRFLSRNNKGKH
QVQLKPMNKSDFEKGEKLIDIFGTVPNSTTQCVKGLNKSNISIFK
VKTDVLGKKHIIKKEGDEPKLKFKRPAATKKAGQAKKKKGSYPYD
VPDYAYPYDVPDYAYPYDVPDYA.

27. The protein of claim 17, wherein the Staphylococcus warneri strain 691 Cas9 has an amino acid sequence at least 80% identical to
(SEQ ID NO: 38)
MPAAKRVKLDGSEVEFSHEYWMRHALTLAKRARDEREVPVGAVLV
LNNRVIGEGWNRAIGLHDPTAHAEIMALRQGGLVMQNYRLYDATL
YVTFEPCVMCAGAMIHSRIGRVVFGVRNAKTGAAGSLMDVLHHPG
MNHRVEITEGILADECAALLCRFFRMPRRVFNAQKKAQSSTDGSS
GSETPGTSESATPESSGPKKKRKVGTMKEKYILGLALGITSVGYG
IINFETKKIIDAGVRLFPEANVDNNEGRRSKRGSRRLKRRRIHRL
DRVKSLLTEYNLINREQIPTSNNPYQIRVKGLSEILSKDELAIAL
LHLAKRRGIHNINVSSEDEDASNELSTKEQINRNNKLLKNKYVCE
VQLQRLKEGQIRGEKNRFKTTDILKEIDQLLKVQKDYHNLDIDFI
NQYKEIVETRREYFEGPGQGSPFGWNGDLKKWYEMLMGHCTYFPQ
ELRSVKYAYSADLFNALNDLNNLIIQRNDSEKLEYHEKYHIIENV
FKQKKKPTLKQIAKEIGVNPEDIKGYRITKSGTPQFTEFKLYHDL
KSIVFDKSILENEAILDQIAEILTIYQDEESIKEELNKLPEILNE
QDKAEIAKLTGYNVTHRLSLKCIHLINEELWQTSRNQMEIFNYLN
IKPNKVDLSEQNKIPKDLVDEFILSPVVKRTFIQSINVINKVIEK
YGIPEDIIIELARENNSDDRKKFINNLQKKNEATRKRINEIIGQT
GNQNGKRIVEKIRLHDQQEGKCLYSLESIPLMDLLNNPQNYEVDH
IIPRSVAFDNSIHNKVLVKQIENSKKGNRTPYQYLNSSDANLSYN
QFKQHILNLSKSKDRISKKKKDYLLEERDINKFEVQKEFINRNLV
DTRYATRELTSYLKAYFSANNMDVKVKTINGSFTNHLRKVWRFDK
YRNHSYKHHAEDALIIANADFLFKENKKLQNANKILEKPTIENDT
QKVTVEKEEDYNNMFETPKLVEDIKQYRDYKFSHRVDKKPNRQLI
KDTLYSTRMKDEHNYIVQTITDIYGKDNTNLKKQFNKNPEKFLMY
QNDPKTFEKLSIIMKQYSDEKNPLAKYYEETGEYLTKYSKKNNGP
IVKKIKLLGNKVGNHLDVTNKYENSTKKLVKLSIKNYRFDVYLTE
KGYKFVTIAYLNVFKKDNYYYIPKDLYQELKAKKKIKDTDQFIAS
FYKNDLIKLNGDLYKIIGVNSDDRNIIELDYYDIKYKDYCEINNI
KGEPRIKKTIGKKTESIEKLTTDVLGNLYLHTTEKAPQLIFKRGL
KRPAATKKAGQAKKKKGSYPYDVPDYAYPYDVPDYAYPYDVPDYA.

28. The protein of claim 17, wherein the Staphylococcus sciuri strain SNUC 2430 Cas9 has an amino acid sequence at least 80% identical to
(SEQ ID NO: 39)
MPAAKRVKLDGSEVEFSHEYWMRHALTLAKRARDEREVPVGAVLV
LNNRVIGEGWNRAIGLHDPTAHAEIMALRQGGLVMQNYRLYDATL
YVTFEPCVMCAGAMIHSRIGRVVFGVRNAKTGAAGSLMDVLHHPG
MNHRVEITEGILADECAALLCRFFRMPRRVFNAQKKAQSSTDGSS
GSETPGTSESATPESSGPKKKRKVGTMKRNYILGLAIGITSVGYG
IIDYETRDVIDAGVRLFKEANVENNEGRRSKRGARRLKRRRRHRL
QRVKKLLFDYNLLTDHSELSGINPYEARVKGLSQKLSEVEFSAAL
LHLAKRRGVHNVNEVEEDTGNELSTKEQISRNSKALEEKYVAELQ
LERLKTDGEVRGPNNRFKTSDYVKEAKQLLKVQKAYHQLDQSFID
TYIDLLETRRTYYEGPGEGSPFGWKDIKEWYEMLMGHCTYFPEEL
RSVKYAYNADLYNALNDLNNLVIARDENEKLEYYEKFQIIENVFK
QKKKPTLKQIAKELLVNEEDIKGYRVTSIGKPEFTNFKIYHDIKG
ITERKEVLENAELLDQIAEILTIYQSSEDVQEELANLNSELTQEE
IEQISNLKGYTGTHNLSLKAINLILDELWHTNDNQMAIFNRLKLV
PKKVDLSQQKEIPTTLVDDFILSPVVKRSFIQSIKVINAIIKKYG
LPNDIIIELAREKNSKDAQKMINEMQKRNRQMNERIEEIIRTTGK
ENAKYLIEKIKLHDMQEGKCLYSLESIPLEDLLNNPFNYEVDHII
PRSVSFDNSFNNKVLVKQEENSKKGNRTPFQYLSSSDSKISYETF
KKHILNLAKGKGRISKTKKEYLLEERDINRFSVQKDFINRNLVDT
RYATRELMNLLRSYFRVNNLDVKVKSINGGFTSFLRRKWKFKKER
NKGYKHHAEDALIIANADFIFKEWKKLDKAKKVMENQTVEEKQAE
SMPEIETEQEYKEIFITPHQIQHIKGFKDYKYSHRVDKKPNRELI
NDTLYSTRKDDKGNTLIVNNLNGLYDKDNDKLKKLMNKSPEKLLM
YHHDPQTYQKLKLIMEQYGDEKNPLYKYYEETGNYLTKYSKKDNG
PVIKKIKYYGKNLKAHLDITDDYPNSRNKVVKLSVKPYRFDVYLD
NDIYKFVTVKNLDVIKKEDYYEVNSKCYKEAKKLKKISDQAEFIA
SFYNNDLIKINGELYRVIGVNNDLLNRIEVNMINITYREYLENMN
DKRPPRIIKTIASKTQSIKKYSTDILGNLYEVKSKQKPQMIMKGK
RPAATKKAGQAKKKKGSYPYDVPDYAYPYDVPDYAYPYDVPDYA.

29. The protein of claim 17, wherein the Streptococcus gallolyticus strain AM24-4 Cas9 has an amino acid sequence at least 80% identical to
(SEQ ID NO: 40)
MPAAKRVKLDGSEVEFSHEYWMRHALTLAKRARDEREVPVGAVLV
LNNRVIGEGWNRAIGLHDPTAHAEIMALRQGGLVMQNYRLYDATL
YVTFEPCVMCAGAMIHSRIGRVVFGVRNAKTGAAGSLMDVLHHPG
MNHRVEITEGILADECAALLCRFFRMPRRVFNAQKKAQSSTDGSS
GSETPGTSESATPESSGPKKKRKVGTMTNGKILGLAIGIASVGVG
IIEAKTGKVVHANSRLFSAANAENNTERRGFRGSRRLNRRKKHRV
KRVRDLFEKHEIVTDFRNLNLSPYELRVKGLTEQLTNEELFAALR
TISKRRGISYLDDAEDDSTGSTDYAKSIDENRRLLKNKTPGQIQL
ERLEKYGQLRGNFTVYDENGEAHRLINVESTSDYEKEARKILETQ
ADYNKKITAEFIDDYVEILTQKRKYYHGPGNEKSRTDYGRFRTDG
TTLENIFGILIGKCNFYPDEYRASKASYTAQEYNFLNDLNNLKVP
TETGKLPTEQKESLVEFAKNTATLGPSKLLKEIAKILDCKVDEIK
GYREDDKGKPDLHTFEPYRKLKFNLESINIDDLSREVIDKLADIL
TLNTEREGIEDAIKRNLPNQFTEEQISEIIKVRKSQSTAFNKGWH
SFSAKLMNELIPELYATSDEQMTILTRLEKFKVNKKSSKNTKTID
EKEVTDEIYNPVVAKSVRQTIKIINAAVKKYGDFDKIVIEMPRDK
NADDEKKFIDKRNKENKKEKDDALKRAAYLYNSSDKLPDEVFHGN
KQLETKIRLWYQQGERCLYSGKPIPIHDLVHNSNNFEIDHILPLS
LSFDDSLANKVLVYDWANQEKGQKTPYQVIDSMDAAWSFREMKDY
VLKQKGLGKKKRDYLLTTENIDKIEVKKKFIERNLIDTRYASRVV
LNSLQSALRELCKDTKVSVIRGQFTSQLRRKWKIDKSRETYHHHA
VDALIIAASSQLKLWEKQDNLMFIDYGNNQVVDKETGEILSVSDD
EYKELVFQPPYQGFVNTISSKGFEDEILFSYQVDSKYNRKVSDAT
IYSTRKAKIGKDKKEETYVLGKIKDIYSQNGFDTFIKKYNKDKTQ
FLMYQKDPLTWENVIEVILRDYPTTKKSEDGKNDVKCNPFEEYRR
ENGLICKYSKKGKGTPIKSLKYYDKKLGNCIDITPEESRNKVILQ
SINPWRADVYFNPETLKYELMGLKYSDLSFEKGTGKYHISQEKYD
AIKEKEGIGKKSEFKFTLYRNDLILIKDTASGEQEIYRFLSRTMP
NVNHYAELKPYDKEKFDGGQELMEVFGKVANGGQCLKSLNKSNIS
IYKVRTDVLGNKYFVKKEGDKPKLNFKNNKKKRPAATKKAGQAKK
KKGSYPYDVPDYAYPYDVPDYAYPYDVPDYA

30. The protein of claim 17, wherein the Lactobacillus kullabergensis strain Biut2 Cas9 has an amino acid sequence at least 80% identical to
(SEQ ID NO: 41)
MPAAKRVKLDGSEVEFSHEYWMRHALTLAKRARDEREVPVGAVLV
LNNRVIGEGWNRAIGLHDPTAHAEIMALRQGGLVMQNYRLYDATL
YVTFEPCVMCAGAMIHSRIGRVVFGVRNAKTGAAGSLMDVLHHPG
MNHRVEITEGILADECAALLCRFFRMPRRVFNAQKKAQSSTDGSS
GSETPGTSESATPESSGPKKKRKVGTMKRVNEDYILGLAIGTNSC
GWAVTDKKNNLLKLRGKTAIGSHLFEEGHTAADRRGFRTTRRRLK
RRKWRLRLLEEIFAEPMAKVDPGFFVRLHQSWVSPLDKDRKKYNA
IVFPTAKEDQAFYKHYATIYHLRDELMTQDRQFDLREIFLAIHHI
VKYRGNFLQDTPVKDFEASKIEVGPILSHINNAFAEKIVEDQDPI
ELNVANAADIEDVIRGKDAEKTVYKLDKVKKIAKLLTDSTAKEEK
NVAKQIANAIMGYKTQFETILDKEIDKSDKAQWEFKLSDADADDK
LDALLPDLDETDQTVVAEIEKLESAITLSTIVDENKSLSQSMVEK
YKKHKKDYKKLKKYINTLQDQTKAKKLLLAYDLYVNNRHGRLLEA
KKTFEDKKKKKALTKDEFYKIVKDNLDDSDLAHEIQQEIAADNFM
PKQRTNSNGVIPFQLHQIELDKIIANQGKYYPFLAAENPVEDHRK
QAPYKLDELVRFRVPYYVGPMITADEQEKTSGKSFAWMVRKEDGQ
ITPWNFEQKVDRQESANKFIKRMTIKDTYLLSEDVLPANSLLYQR
FEVLNELNNIRINGSRISVDLKQQIFNDLFEEKKTVTEKSLTSYL
KQNLHLPTVEIKGLADPTKFNSSLASYYHLKSLHVFDKELADPQY
QKDFEKIIEYSSIFEDKKIFQDKLHAEFKWLTPEQFKAISTWRLQ
GWGRLSRKLLVELHDTNGQNIMEQLWDSQKNFMQIVTEPDFKDAI
AKENQNVTRANGVEEILADAYTSPANKKAIRQVVKVVADVVKAAG
GKKPAQFAIEFTRDPDKNPQLSHIRGTKLLKAYQETAGELVDQKL
TDSLKEAMTSRKLLKDKYFLYFMQAGRDAYTGQKINIDEVSTNYQ
IDHILPQSFIKDDSFDNRVLTATPLNAEKSDDVPYKRFANNYVSD
MKMTVGEMWKHWQKAGIINKHKLGNLLLDPDRLNKFQKSGFINRQ
LVETSQIIKLVSVILQNKYPDAEIITVKAGDNSALRQRLNLYKSR
DVNDYHHAIDAYLSIICGNFLYQVYPKYRPYFVYGKYKKFSQDPD
LQKEVIKHFKGFTFMWPLLQKDNSERKAPEKIKENNSDRIVFYKH
PDIFDKLRKAYNYKYMLVSRETTTENSGLFDVTIYPRGERDLAKT
RKLIPKSNGLDPKIYGGYSGNTDAYMVIVKIDKGKESIYKVIGVP
MRALASLNRAKKQGNYKEELHQVLEPQIMFDKNGKPKRSVKGFRI
IKDHVPFKQVVLDGDKKFMLNSSTYEINAKQLTLTPETMRIVTDN
LKKGEDQDQLLVKAYDEILQKVDQYLPLFDVNKFRNSLHLGRAKF
LDLAVNDKKITLTNILNGLHDNLVTPDLKNIGIKTPLGKLQVPSG
IVLSSEAILIFQSPTGLFEKRVRIADLKRPAATKKAGQAKKKKGS
YPYDVPDYAYPYDVPDYAYPYDVPDYA.

31. The protein of claim 17, wherein the Streptococcus suis strain LSS83 Cas9 has an amino acid sequence at least 80% identical to
(SEQ ID NO: 42)
MPAAKRVKLDGSEVEFSHEYWMRHALTLAKRARDEREVPVGAVLV
LNNRVIGEGWNRAIGLHDPTAHAEIMALRQGGLVMQNYRLYDATL
YVTFEPCVMCAGAMIHSRIGRVVFGVRNAKTGAAGSLMDVLHHPG
MNHRVEITEGILADECAALLCRFFRMPRRVFNAQKKAQSSTDGSS
GSETPGTSESATPESSGPKKKRKVGTMSNGKILGLAIGIASVGVG
VIDAQTGEIIHASSRIFPSANAANNAERRTFRGSRRLIRRKKHRI
KRLDDLFNDFHINLDGEMSTDNPYVLRVKGLSQKLTVEELYISIK
NIMKRRGISYLDDAESDNEAGRSDYAKAIERNRQLLTSKTPGEIQ
LERLEKYGQLRGNFTIIDEEGQSQQIINVESTSDYVKEVEKILDC
QKMYHKFISDEFCDKLIELLREKRKYYVGPGNEKSRTDYGIYRTD
GTTLENLFGILIGKCTFYPDQYRSSRASYTAQEFNFLNDLNNLTV
PTETKKLSQEQKEFLVNYAKETSVLGAGKILQQIAKLADCKVEDI
RGYRLDNKDKPELHTFETYRAMKGLVPLVDIGVLSREQLDILADI
LTLNTDFEGIREALKKQLPNVFDEKQVKGLASFRKSKSQLFAKGW
HNLSQKIMLEVIPELYATSDEQMTILTRLGKFEKSSVAEYPSSIN
VDEITDEIYNPVVAKSIRQTIKIINASIKKWDEFDQIVIEMPRDR
NEDEEKKRIADGQKANAKEKADSILRAAELYCAGKVLPDYVYNGH
NQLATKIRLWYQQGERCIYTGQPISIHDLIHNQNQYEIDHILPLS
LTFDDSLSNKVLVLATANQEKAQRTPYNYLKSATSAWSYREFKDY
VTKRKGIGKKKCEYLTFEEDINGFEVRSKFIQRNLVDTRYASKVI
LNALQDYFKISGIQTKVSVVRGQFTSQLRHKWGIEKTRETYHHHA
VDALIIAASSQLRLWKKQESPLVVDYQEGRQVDLETGEILELTDE
QYKELVYQPPYQGFVNTISSSAFDNEILFSYQVDSKVNRKISDAT
IYATRNAQLGKDKTEGIYVLGKIKDIYTQAGYEAFLKRYTKDKTS
FLMYHKDLDTWEKVIEIILRDYREYDEKGKEIGNPFERYRRENGY
VKKYSRKGNGTAIKSLKYYDNKLGNHIDITPENSRNAVVLQSLKP
WRTDVYFNKETGKYEFLGIKYSDLSFEKGTGEYGISQEKYDSIKI
AEGVAKKSIFKFTLYKQDLLFIKDIENNFGKLLRFTSKNDTSKHY
VELKPYDKNKFGTEEPLLPVLGNVAKSGQCIKGLNKSNISIYKVR
TDILGYRHFIKQEGEHPQLKFKKKRPAATKKAGQAKKKKGSYPYD
VPDYAYPYDVPDYAYPYDVPDYA

32. The protein of any one of the preceding claims, wherein the amino acid sequence of the Cas9 protein comprises at least one, at least two, at least three, at least four, at least five, at least six, at least seven, at least eight, at least nine, or at least 10 mutations in SEQ ID NO: 1, 2, 3, 4, 5, 6, 7, 8, 9, 10, 11, 12, 13 or 14.

33. The protein of claim 32, wherein the mutation is an amino acid substitution.

34. The protein of any one of the preceding claims, wherein the Cas9 protein has nickase activity.

35. The protein of claim 32, wherein the at least one mutation results in an inactive Cas9 (dCas9).

36. The protein of any one of the preceding claims, wherein the Cas9 protein comprises at least one amino acid mutation in PAM Interacting, HNH and/or RuvC domain.

37. An engineered, non-naturally occurring Cas9 fusion protein comprising a Cas9 protein having at least 80% identity to SEQ ID NOs: 1, 2, 3, 4, 5, 6, 7, 8, 9, 10, 11, 12, 13 or 14 and wherein the Cas9 protein is fused to a histone demethylase, a transcriptional activator, or to a deaminase.

38. The Cas9 protein of claim 37, wherein the Cas9 protein is fused to a cytosine deaminase or to an adenosine deaminase.

39. The Cas9 protein of claim 38, wherein the Cas9 protein is fused to a adenosine deaminase and has an amino acid sequence at least 80% identical to
(a)
(SEQ ID NO: 29)
MPAAKRVKLDGSEVEFSHEYWMRHALTLAKRARDEREVPVGAVLV
LNNRVIGEGWNRAIGLHDPTAHAEIMALRQGGLVMQNYRLYDATL
YVTFEPCVMCAGAMIHSRIGRVVFGVRNAKTGAAGSLMDVLHHPG
MNHRVEITEGILADECAALLCRFFRMPRRVFNAQKKAQSSTDGSS
GSETPGTSESATPESSGPKKKRKVGTMEKSYSIGLAIGTNSVGWS
VITDDYKVPAKKMRVLGNTDKKYIKKNLLGALLFDSGETAEATRL
KRTARRRYTRRKNRLRYLQEIFAKEMAKVDESFFQRLDESFLTDD
DKTFDSHPIFGNKAEEDAYHQKFPTIYHLRKHLADSQEKADLRLI
YLALAHMIKFRGHFLYDNFNDDNFDWRNIDIQKRYEEFIETYDST
LGESYLADISVDAASILEEKVSKTERLENLLKYYPTEKKTTFFGN
LIKLILGQQAKFKAIFNLEDEISLQFATPTYDEDLEELLGKIDNG
DSYSELFVAAQNLYNTILLASFLKTDNKSAKAPLSTSMIERYENH
KKDLAKLKDFVKKNCPDQYHDIFRDKSKNGYAGYIDNGVSQNDFY
TFIGKCLEESLKKDKGAQYFLDKIDRDDFLRKQRTFDNGAFPYQI
HLQEMHAILRRQGDYYPFLKENQDKIEKILTFRIPYYVGPLARKD
SRFAWANYRSDEAITPWNFDEVVDKEKSAEKFITRMTLNDLYLPE
EKVLPKHSLIYETFTVYNELTNIKYVNDQGNAIHFDSELKEKIFN
QLFKENRKVSKKTLIDFLNNSEGIYTDKLVGIDEEVKYLNASLGT
YHDLKKILESFMDDEINEKIIEDIIQTLTLFEDIEMKRQRLQKYD
DIFTPKQLKELARRNYTGWGRLSYKLINGIRNKENNKTILDYLKN
GNRNFMQLINDDRLSFKQIIIDARKIEKLDNIESVVYNLPGSPAI
KKGILQSIKIVDELVKVMGHNPDNIVIEMARENQTTNQGKNRSQQ
RLKRLQDSMSNFKDSSISLKDVDNSDLQNDRLFLYYIQNGKDMYT
GEELDIDHLSDYDIDHIIPQSFIKDNSIDNRVLTSSAKNRGKSDD
VPGRDVVLKMKPFWKKLYDVKLISKRKFDNLTKSEHGGLTESDKA
GFIKRQLVETRQITKYVAQILDGRFNTKRDDNNKVIRDVKVITLK
SSLVSQFRKDFGFYKVREINDYHHAHDAYLNAVVGTAILKKYPKL
APEFVYGEYKKCDVRKLIAKSGDKSEIGKATAKYFFYSNLMNFFK
RVIRYSNGMIVVRPVIEYSKDTGEIAWDKEKDFKTVCKVLSCPQV
NIVKKVEKQSHGLDRGKPKGFYNANPSPKPKKGSKVNLVPIKANL
NPKNYGGYAGISNSYAVLVDATIEKGAKKKLTRIQEFQGISIIDR
EKYEKNKVEFLKGLGYKEIYSIITLPKYSLFELADGSRRMLASIL
STNNKRGEIHKGNELVLPAKYIPLLYHANRIHNTFETGHREYVEK
HIAEFKEIAEIILEFNNKYVNAKKNSSIIEKALESFDSFSLDEIC
DSFVGKLKKNNTKKNSGLFELVSLGSASDFEFLETKVPRYRDYTP
SSLLNATLIHQSITGLYETRIDLSKLGEEKRPAATKKAGQAKKKK
GSYPYDVPDYAYPYDVPDYAYPYDVPDYA.
(b)
(SEQ ID NO: 30)
MPAAKRVKLDGSEVEFSHEYWMRHALTLAKRARDEREVPVGAVLV
LNNRVIGEGWNRAIGLHDPTAHAEIMALRQGGLVMQNYRLYDATL
YVTFEPCVMCAGAMIHSRIGRVVFGVRNAKTGAAGSLMDVLHHPG
MNHRVEITEGILADECAALLCRFFRMPRRVFNAQKKAQSSTDGSS
GSETPGTSESATPESSGPKKKRKVGTMTKDYTIGLAIGTNSVGWA
VLTDDYQLMKRKMSVHGNTEKKKIKKNFWGARLFDEGQTAEFRRT
KRTNRRRLARRKYRLSKLQDLFAEELCKQDDCFFVRLEESFLVPE
EKQYKPASIFPTLEEEKEYYQKYPTIYHLRQKLVDSTEKEDLRLV
YLALAHLLKYRGHELFEGDLDTENTSIEESFRVFLEQYSKQSDQP
LIVHQPVLTILTDKLSKTKKVEEILKYYPTEKINSFFAQCLKLIV
GNQANFKRIFDLEAEVKLQFSKETYEEDLESLLEKIGDEYLDIFL
QAKKVHDAILLSEIISSTVKHTKAKLSSGMVERYERHKADLAKFK
QFVKENVPQKSTAFFKDTTKNGYAGYIKGKTTQEEFYKFVKKELS
GVVGSEPFLEKIDQETFLLKQRTYTNGVIPHQVHLIELKAIIDQQ
KQHYPFLEEAGPKIIALFKFRIPYYVGPLAKEQEASSFAWIERKT
AEKIHPWNFSEVVDIEKSAMRFIQRMTKQDTYLPTEKVLPKNSLL
YQKYMIFNELTKVSYKDERGVKQYFSGDEKQQIFKQLFQKERGKI
TVKKLQNFLYTHYHIENAQIFGIEKAFNASYSTYHDFMKLAKTNQ
KAMQEWLEQPEMEPIFEDIVKILTIFEDRQMIKHQLSKYQEVFGE
KLLKEFARKHYTGWGRFSAKLIHGIRDRKTNKTILDYLINDDDVP
ANRNRNLMQLINDEHLSFKEEIAKATVFSKHKSLVDVIQDLPGSP
AIKKGIWQSLKIVEELIAIIGYKPKNIVIEMARENQKTHRTKPRL
KALENGLKQIGSTLLKEQPTDNKALQKERLYLYYLQNGRDMYTGE
PLEIENLHQYEVDHIIPRSFIVDNSIDNKVLVARKQNQKKRDDVP
KKQIVNEQRIFWNQLKEAKLISPKKYAYLTKIELTPEDKARFIQR
QLVETRQITKHVANILHQSFHQEEEGTDCDGVQIITLKATLTSQF
RQTFGLYKVREINPHHHAHDAYLNGFIANVLLKRYPKLAPEFVYG
KYVKYSLARENKATAKKEFYSNILKFFESDEPFCDENGEIYWEKS
HHLPRIKKVLSSHQVNVVKKVEQQKGGFYKETVNSKEKPDKLIER
KNNWDVTKYGGFGSPVIAYAIAFVYAKGKTQKKTRAIEGITIMEQ
AAFEKDPTTFLKEKGFPQVTEFIKLPKYTLFEFDNGRRRFLASHK
ESQKGNPFILSDQLVTLLYHAQHYDKITYQESFDYVNTHLSDFSA
ILTEVLAFAEKYTLADKNIERIQELYEENKYGEISMIAQSFLQLL
QFNAIGAPADFKFFGVTIPRKRYTSLTEIWDATIIYQSVTGLYET
RIRMGDLWAGEQKRPAATKKAGQAKKKKGSYPYDVPDYAYPYDVP
DYAYPYDVPDYA
(c)
(SEQ ID NO: 31)
MPAAKRVKLDGSEVEFSHEYWMRHALTLAKRARDEREVPVGAVLV
LNNRVIGEGWNRAIGLHDPTAHAEIMALRQGGLVMQNYRLYDATL
YVTFEPCVMCAGAMIHSRIGRVVFGVRNAKTGAAGSLMDVLHHPG
MNHRVEITEGILADECAALLCRFFRMPRRVFNAQKKAQSSTDGSS
GSETPGTSESATPESSGPKKKRKVGTMTNGKILGLAIGVASVGVG
IIEAKTGKVIHANSRLFSAANAENNAERRGFRGARRLTRRKKHRV
KRVRDLFEKYDISTDFRNLNLNPYELRVKGLSEQLTNEELFAALR
TIAKRRGISYLDDAEDDSTGSSDYAKSIDENRRLLKSMTPGQIQL
ERLEKYGQLRGNFTVYDENGEAHRLINVESTSDYKNEARKILETQ
SNYNKQITDEFIEDYIEILTQKRKYYHGPGNEKSRTDYGRFRTDG
TTLENIFGILIGKCSFYPEEYRASKASYTAQEFNFLNDLNNLKVP
TETGKLSTEQKEYLVDFAKNSKTLGASKLLKEIAKLVDGDVKDIS
GYREDSKGKPDLHTFEPYRKLKFNLTTVDIDNLSRDILDKLANIL
TLNTEREGIEDAINRNLPEQFTKEQISEIVQIRKSQSSAFNKGWH
SFSAKLMNELIPELYATSEEQMTILTRLEKFKATKKSSKNTKTID
EKEITDEIYNPVVAKSVRQTIKIINAAVKKYGDFDKIVIEMPRDK
NADDEKKFIDKKNKENKKEKDDSLKRAAYLYNGTDKLPDDVFHGN
KQLATKIRLWYQQGERCLYSGKPILIQDLVHNSNNFEIDHILPLS
LSFDDSLANKVLVYAWTNQEKGQKTPYQVIDSMDAAWSFREMKDY
VLKQKGIGKKKREYLLTTENIDKIEVKKKFIERNLVDTRYASRVV
LNSLQTALKELGKDTKVSVVRGQFTSQLRRKWKIDKSRETYHHHA
VDALIIAASSQLKLWQKHENLMFENYGENQVVNKETGEILSISDD
EYKELVFQPPYQGFVNTISSKAFEDEILFSYQVDSKFNRKVSDAT
IYSTRKAKLGKDKKEETYVLGKIKDIYSQDGFDTFIKRYKKDKTQ
FLMYQKDPLTWENVIEVILRDYPTTKKSEDGKNDVKCNPFEEYRR
ENGLICKYSKKGKGTPIKSLKYYDKKLGNHISITPKESKNDVVLQ
SLNPWRADLYFNPDTLKYELMGLKYSDLSFEKGTGKYHISQEKYD
EIKEKEGIGQNSEFKFTLYRNDLILIKDTESGEQEIYRFLSRTMP
NVKHYVELKPYDKEKFNGGQELIKSLGEADKVGRCLKGLSKPGIS
IYKVRTDVLGNKFFVKKEGDKPKLDFKNNKKKRPAATKKAGQAKK
KKGSYPYDVPDYAYPYDVPDYAYPYDVPDYA.
(d)
(SEQ ID NO: 32)
MPAAKRVKLDGSEVEFSHEYWMRHALTLAKRARDEREVPVGAVLV
LNNRVIGEGWNRAIGLHDPTAHAEIMALRQGGLVMQNYRLYDATL
YVTFEPCVMCAGAMIHSRIGRVVFGVRNAKTGAAGSLMDVLHHPG
MNHRVEITEGILADECAALLCRFFRMPRRVFNAQKKAQSSTDGSS
GSETPGTSESATPESSGPKKKRKVGTMNGLVLGLAIGIASVGVGI
LNKETGEIIHVNSRIFPAATADSNVERRGFRQGRRLGRRKKHRSA
RLNDLFEEFGFITDESAVPLNLNPYALRVKGLSEELTNEELFIAL
KNIIKRRGISYLDDASEDGETASNEYGKAVEENRKLLADKTPGQI
QLERFEKYGQVRGDFTVVENGENHRLINVESTSAYKKEAERILRR
QQEFNVRISDEFIEAYLTILTGKRKYYHGPGNEKSRTDYGRFRTD
GTTLDNIFGILIGKCTFYPDEYRAAKASYTAQEFNLLNDLNNLTV
PTETKKLRPEQKRQIVEYARTAKTLGTPTLLKYIAKLVDGSIDDI
KGYRIDKSDKPEMHTFDAYRKMRTLELVDVDILSRETLDDLAYIL
TLNTESEGILEALNSKMPGTFTKEQIDELIQFRKKNSAVFGKGWH
NFSLKLMNELISELYETSEEQMTILTRLGKQRSREISKRTKYIDE
KELTDEIYNPVVAKSVRQAIKIINLATKKYGIFDNIVIEMARESN
EDDEKKAIQNVQKANEDEKKAAMEKAADLYNGKKELPDSIFHGHK
ELATKIRLWHQQGERCLYTGKNISIHDLIHNPHQYEIDHILPLSL
SFDDGLANKVLVLATANQEKGQRTPFQALDSMDDAWSYIEFKQYV
RNSKSLSNKKKDYLLTEEDISKIEVKQKFIERNLVDTRYSSRVVL
NTLQEFYKTNDFDTKISVVRGQFTSQLRRKWKIEKSRDTYHHHAV
DALIIAASSQLRLWKKQNNPLISYREGQLVDPETGEILSLTDDEY
KELVFRPPYDYFVDTLKSKSFEDSILFSYQVDSKYNRKISDATIY
GTRKAQLGKDKQEETYVLGKIKDIYSQKGYEDFIKRYKKDTTQFL
MYHKDPQTFAKVIEEILKTYPDKELNEKGKEIPCNPFEKYRQENG
PIRKYSKKGKGPEIKSLKYYDNKLGNHIDITPVNSQNQVVLQSLK
PWRTDVYFNPQTSKYELMGLKYSDLRFEKGSGSYGISPEKYNKVK
AKEGVDEDSEFKFTLYKNDLILIKDTETGEQQLFRYGSRNDTSKH
YVELKPYEKAKFEGNQQLMNLLGTVAKGGQCLKGINKPNLSIYKV
KTDVLGNKHFIKKEGDQPQLNFKKKIKRPAATKKAGQAKKKKGSY
PYDVPDYAYPYDVPDYAYPYDVPDYA.
(e)
(SEQ ID NO: 33)
MPAAKRVKLDGSEVEFSHEYWMRHALTLAKRARDEREVPVGAVLV
LNNRVIGEGWNRAIGLHDPTAHAEIMALRQGGLVMQNYRLYDATL
YVTFEPCVMCAGAMIHSRIGRVVFGVRNAKTGAAGSLMDVLHHPG
MNHRVEITEGILADECAALLCRFFRMPRRVFNAQKKAQSSTDGSS
GSETPGTSESATPESSGPKKKRKVGTMNNSYILGLAIGITSVGYG
IIEYETRDVIDAGVRLFKEANVENNEGRRSKRGARRLKRRRRHRL
QRVKKMLFDYKLLNEDSEISGINPYEARVKGLSEKLSDEEFSAAL
LHLAKRRGVHNVSDVEEDTGNELSTKEQIARNNKALEDKYVAELQ
LERLKEQGEVRGAANRFKTSDYIKEAKQLLKTQSDYHKIDETFIE
TYISLLETRRTYYEGPGEGSPFGWKDIKEWYEMLMGHCTYFPEEL
RSVKYAYNADLYNALNDLNNLVIARDENEKLEYYEKFQIIENVFK
QKKKPTLKQIAKEILVNEEDIKGYRVTSTGKPEFTNFKIYHDIKG
ITSRKEILENADLLNQIAEILTIYQSAEDIQEELAELEPKLTQEE
IEQISNLTGYTGTHRLSLKAINLILDELWNTSDNQMTIFNRLKLV
PKKVDLSQQKEIPTSLVDDFILSPVVKRSFIQSIKVINAIIKKFG
LPKDIIIELAREKNSKEAQKFINEMQKRNRQTNERIEKIIKETGK
EKAKFLIEKIKLHDMQEGKCLYSLESIPLEDLLNNPYHYEVDHII
PRSVSFDNSFNNKVLVKQEENSKKGNRTPFQYLSSSDAKISYETF
KKHILNLSKGKGRVSKKKKEYLLEERDINRFSVQKDFINRNLVDT
RYATRELMNLLRSYFRVNDLDVKVKSINGGFTSFLRTKWKFKKER
NQGYKHHAEDALVIANADFIFKEWKKLDTTNKVMENQTVEEQQAE
NMPGIETDDEYKEIFVIPRQIQSIKDFKDYKYSHRVDKKPNRELV
NDTLYSTRKDDKGNTLIINNIKGLYDKDNDKLKNLIKKSPEKLLM
YHHDPQTYQKLKTIMEQYSNEKNPLYKYHEETGNYLTKYSKKDNG
PIIKKVKYYGKKLNAHLDITNDYSNSQNKIVKLSLKPYRFDVYLD
NGGYKFVTVKNLDVIKKEGFFKIDSNAYEKAKSEKKIDENAVFIA
SFYNNDLIKIDGELYRIVGVNNDTRNVVELNMIPITYKEYLENIN
DKRTPRILKTISQKTYSIEKYSTDILGNLYKVKSKKKPQMIMKGK
RPAATKKAGQAKKKKGSYPYDVPDYAYPYDVPDYAYPYDVPDYA.
(f)
(SEQ ID NO: 34)
MPAAKRVKLDGSEVEFSHEYWMRHALTLAKRARDEREVPVGAVLV
LNNRVIGEGWNRAIGLHDPTAHAEIMALRQGGLVMQNYRLYDATL
YVTFEPCVMCAGAMIHSRIGRVVFGVRNAKTGAAGSLMDVLHHPG
MNHRVEITEGILADECAALLCRFFRMPRRVFNAQKKAQSSTDGSS
GSETPGTSESATPESSGPKKKRKVGTMENKNYSIGLAIGTNSVGW
AVITDDYKVPSKKMKVFGNTDKHFIKKNLIGALLFDEGATAEDRR
LKRTARRRYTRRKNRLRYLQEIFSEEISKLDSSFFHRLDDSFLVP
KDKRGSKYPIFATLEEEKEYHKKFPTIYHLRKHLADSKEKTDLRL
IYLALAHMIKYRGHFLYEESFDIKNNDIQKIFNEFISIYDNTFEG
SSLSGQNAQVEAIFTDKISKSAKRERVLKLFSDEKSTSLFSEFLK
LIVGNQADFKKHFDLEEKAPLQFSKDTYDEDLENLLGQIGDGFTD
LFLVAKKLYDAILLSGILTVTDPSTKAPLSASMIERYESHQKDLA
ALKQFIKNNLPKRYNEVESDQSKDGYAGYIDGKTTQEAFYKYIKN
LLSKFEGADYFLDKIEREDFLRKQRTFDNGSIPHQIHLQEMNAIL
RRQGEHYPFLKENREKIEKILTFRIPYYVGPLARGNRDFAWLTRN
SDQAIRPWNFEEIVDKASSAEEFINKMTNYDLYLPEEKVLPKHSL
LYETFAVYNELTKVKFIAEGLRDYQFLDSGQKKQIVNQLFKEKRK
VTEKDIIHYLHNVDGYDGIELKGIEKQFNASLSTYHDLLKIIKDK
EFMDDPKNEEILENIVHTLTIFEDREMIKQRLAQYDSIFDEKVIK
ALTRRHYTGWGKLSAKLINGICDKQTGDTILDYLIDDGKINRNFM
QLINDDGLSFKEIIQKAQVVGKTDDVKQVVQELPGSPAIKKGILQ
SIKIVDELVKVMGYAPESIVIEMARENQTTARGKKNSQQRYKRIE
DSLKNLAPGLDSNILKENPTDNIQLQNDRLFLYYLQNGKDMYTGK
PLDIDQLSSYDIDHIIPQAFIKDDSIDNRVLTSSKDNRGKSDNVP
SLEVVQKRKAFWQQLLDSKLISERKFNNLTKAERGGLDERDKVGF
IRRQLVETRQITKHVAQILDASFNTEVNEKNQKIRTVKIITLKSN
LVSNFRKEFELYKVREINDYHHAHDAYLNAVVAKAILKKYPKLEP
EFVYGDYQKYDLKRYISRFKPSKEIEKATEKYFFYSNLLNFFKEE
VHYADGIIVKRENIEYSKDTGEIAWNKEKDFATIKKVLSYPQVNI
VKKTEIQTHGLDRGKPKGLFNSNPSPKPSEDSKENLVPIKQGLDP
RKYGGYAGISNSYAVLVKAIVEKGAKKQQKTILEFQGISILDKIN
FENNKENYLLKKRYIEILSTITLPKYSLFEFPDGTRRRLASILST
NNKRGEIHKGNELVLPGKYTTLLYHAKNINKKLEPEHLEYVEKHR
NDFAKLLECVLNFNDKYVGALKNGERIRQAFTDWETVDIEKLCFS
FIGPENSKNAGLFELTSQGSASDFEFLGVKIPRYRDYAPSSLLKA
TLIHQSITGLYETRIDLSKLGEDKRPAATKKAGQAKKKKGSYPYD
VPDYAYPYDVPDYAYPYDVPDYA.
(g)
(SEQ ID NO: 35)
MPAAKRVKLDGSEVEFSHEYWMRHALTLAKRARDEREVPVGAVLV
LNNRVIGEGWNRAIGLHDPTAHAEIMALRQGGLVMQNYRLYDATL
YVTFEPCVMCAGAMIHSRIGRVVFGVRNAKTGAAGSLMDVLHHPG
MNHRVEITEGILADECAALLCRFFRMPRRVFNAQKKAQSSTDGSS
GSETPGTSESATPESSGPKKKRKVGTMSDLVLGLAIGIGSVGVGI
LNKVTGEIIHKNSRIFPAAQAENNLERRTNRQGRRLTRRKKHRRV
RLNHLFEESGLITDFTKVSINLNPYQLRVKGLTAELSNEELFIAL
KNMVKHRGISYLDDASDDGNSSVGDYAQIVKENSKQLETKTPGQI
QLERYQKYGQLRGDFTVEEDGRKHRLINVFPTSAYHAEALRILQT
QQEFNPQITDEFINSYLEILTGKRKYYHGPGNEKSRTDYGKYTTK
KDAQGQYITLNNIFGILIGKCTFYPEEYRAAKASYTAQEFNLLND
LNNLTVPTETKKLSEEQKYQIITYVKNEKAMGPAKLFKYIAKLLS
CDVADIKGYRIDKSDKAEIHTFEAYRKMKTLETLDVKKMAREELD
KLAYVLTLNTEREGIQEALDHEFADGTFSQEQVDELVQFRKANSS
IFGKGWHSFSVKLMMELIPELYATSEEQMTILTRLGKQKTTSSSN
KTKYIDEKQLTEEIYNPVVAKSVRQAIKIVNAAIKKYGDFDNIVI
EMARETNEDDEKKAIQKIQKANKAEKDAAMRKAANQYNGKAELPH
SVFHGHKQLATKIRLWHQQGERCLYTGKTISIHDLINNPNQFEID
HILPLSITFDDSLANKVLVYATANQEKGQRTPYQALDSMDDAWSF
RELKAFVRESKALSNKKKEYLLTEEDISKFDVRKKFIERNLVDTR
YASRVVLNALQEHFRTHKIDTKVSVVRGQFTSQLRRHWGIEKTRD
TYHHHAVDALIIAASSQLNLWKKQKNTLVSYSEDQLLDIETGELI
SDDEYKESVFKAPYQHFVDTLKSKEFEDSILFSYQVDSKFNRKIS
DATIYATRKAKLDKEKKEYTYTLGKIKDIYALGTKTPSKTGFYKF
LDLYKKDKSQFLMYQKDRRTWDEVIEKILEQYRPFKEKDKNGKEV
DFNPFEKYRIENGPIRKYSRKGNGPEIKSLKYYDNLLGRFVDITP
SESKNPVALLSLNPWRTDVYYNTETRKYEFLGLKYADLCFEKGGS
YGISKVKYNKIREKEGIGKNSEFKFTLYKNDLILIKDTETNRQQI
FRFWSRTGKDNPKSFEKHKLELKPYEKTRFEKGEELKVLGKVPPS
SNRLQKNMQIENLSIYKVRTDVLGNQHIIKNEGDKPKLDFKRPAA
TKKAGQAKKKKGSYPYDVPDYAYPYDVPDYAYPYDVPDYA.
(h)
(SEQ ID NO: 36)
MPAAKRVKLDGSEVEFSHEYWMRHALTLAKRARDEREVPVGAVLV
LNNRVIGEGWNRAIGLHDPTAHAEIMALRQGGLVMQNYRLYDATL
YVTFEPCVMCAGAMIHSRIGRVVFGVRNAKTGAAGSLMDVLHHPG
MNHRVEITEGILADECAALLCRFFRMPRRVFNAQKKAQSSTDGSS
GSETPGTSESATPESSGPKKKRKVGTMNGLVLGLAIGIASVGVGI
LEKNSGKIIHANSRIFPAATADNNVERRKNRQARRLHRRKKHRGV
RLQDIFEDYGLLTDFSKVSINLNPYRLRVDGLDQQLTNEELFIAL
KNIVKRRGISYLDDASEDGGTVSSDYGKAVEENRKLLAEKTPGQI
QLERFEKYGQVRGDFTVVENGEKRRLINVESTSAYRKEAERILRK
QQEFNSKITDEFIEDCLKILTGKRKYYHGPGNEKSRTDYGRFRTD
GTTLDNIFGILIGKCTFYPNEYRASKASHTAQEFNLLNDLNNLTV
PTETKKLSEEQKKVIVEYAKEAKTLGASTLLKYIAKMIDASVDQI
SGYRVDVNNKPEMHTFEVYRKMQSLETISVGELSRNVLDELAHIL
TLNTEREGIEEAINTKLKDSFSQDQVLELVQFRKNNSSLFSRGWH
NFSLKLMMELIPELYETSEEQMTILTRLGKQKSKETSKRTKYIDE
KELTEEIYNPVVAKSVRQAIKIINEATKKYGIFDNIVIEMARENN
EEDAKKDYIKRQKANQDEKNAAMEKAAFQYNGKKELPDNIFHGHK
ELATKIRLWHQQGEKCLYTGKNIPISDLIHNQYKYEIDHILPLSL
SFDDSLSNKVLVLATANQEKGQRTPFQALDSMDDGWSYREFKSYV
KESKLLGNKKKEYLLTEEDISKIEVKQKFIERNLVDTRYSSRVVL
NALQDFYKAHKFDTTISVVRGQFTSQLRRKWGLEKSRETYHHHAV
DALIIAASSQLRLWKKQNNPLIAYKEGQFVDSETGEILSLSDDEY
KELVFKAPYDHFVDTLRSKTFEDSILFSYQVDSKYNRKISDATIY
ATRKAKLDKDKSEETYVLGKIKDIYSQAGYDAFIKIYNKDKSKFL
MYHKDPQTFEKVIEEILRTYPSKELNDKNKEIPCNPFEKYRQENG
PIRKYSKKGNGPEIKCLKYYDNKLGNYIDITPDGSDNQVVLQSIA
PWRTDVYYNHKTGKYEFLGLKYSDLYFEKGTGKYKISKEKYDNIK
KIEGVVETSEFKFTLYKNDLILIKDVEKGQEQLFRFLSRNNKGKH
QVQLKPMNKSDFEKGEKLIDIFGTVPNSTTQCVKGLNKSNISIFK
VKTDVLGKKHIIKKEGDEPKLKFKRPAATKKAGQAKKKKGSYPYD
VPDYAYPYDVPDYAYPYDVPDYA.
(i)
(SEQ ID NO: 37)
MPAAKRVKLDGSEVEFSHEYWMRHALTLAKRARDEREVPVGAVLV
LNNRVIGEGWNRAIGLHDPTAHAEIMALRQGGLVMQNYRLYDATL
YVTFEPCVMCAGAMIHSRIGRVVFGVRNAKTGAAGSLMDVLHHPG
MNHRVEITEGILADECAALLCRFFRMPRRVFNAQKKAQSSTDGSS
GSETPGTSESATPESSGPKKKRKVGTMNGLVLGLAIGIASVGVGI
LEKNSGKIIHANSRIFPAATADNNVERRKNRQARRLHRRKKHRGV
RLQDIFEDYGLLTDFSKVSINLNPYRLRVDGLDQQLTNEELFIAL
KNIVKRRGISYLDDASEDGGTVSSDYGKAVEENRKLLAEKTPGQI
QLERFEKYGQVRGDFTVVENGEKRRLINVESTSAYRKEAERILRK
QQEFNSKITDEFIEDCLKILTGKRKYYHGPGNEKSRTDYGRFRTD
GTTLDNIFGILIGKCTFYPNEYRASKASHTAQEFNLLNDLNNLTV
PTETKKLSEEQKKVIVEYAKEAKTLGASTLLKYIAKMIDASVDQI
SGYRVDVNNKPEMHTFEVYRKMQSLETISVGELSRNVLDELAHIL
TLNTEREGIEEAINTKLKDSFSQDQVLELVQFRKNNSSLFSRGWH
NFSLKLMMELIPELYETSEEQMTILTRLGKQKSKETSKRTKYIDE
KELTEEIYNPVVAKSVRQAIKIINEATKKYGIFDNIVIEMARENN
EEDAKKDYIKRQKANQDEKNAAMEKAAFQYNGKKELPDNIFHGHK
ELATKIRLWHQQGEKCLYTGKNIPISDLIHNQYKYEIDHILPLSL
SFDDSLSNKVLVLATANQEKGQRTPFQALDSMDDGWSYREFKSYV
KESKLLGNKKKEYLLTEEDISKIEVKQKFIERNLVDTRYSSRVVL
NALQDFYKAHKFDTTISVVRGQFTSQLRRKWGLEKSRETYHHHAV
DALIIAASSQLRLWKKQNNPLIAYKEGQFVDSETGEILSLSDDEY
KELVFKAPYDHFVDTLRSKTFEDSILFSYQVDSKYNRKISDATIY
ATRKAKLDKDKSEETYVLGKIKDIYSQAGYDAFIKIYNKDKSKFL
MYHKDPQTFEKVIEEILRTYPSKELNDKNKEIPCNPFEKYRQENG
PIRKYSKKGNGPEIKCLKYYDNKLGNYIDITPDGSDNQVVLQSIA
PWRTDVYYNHKTGKYEFLGLKYSDLYFEKGTGKYKISKEKYDNIK
KIEGVVETSEFKFTLYKNDLILIKDVEKGQEQLFRFLSRNNKGKH
QVQLKPMNKSDFEKGEKLIDIFGTVPNSTTQCVKGLNKSNISIFK
VKTDVLGKKHIIKKEGDEPKLKFKRPAATKKAGQAKKKKGSYPYD
VPDYAYPYDVPDYAYPYDVPDYA.
(j)
(SEQ ID NO: 38)
MPAAKRVKLDGSEVEFSHEYWMRHALTLAKRAR
DEREVPVGAVLVLNNRVIGEGWNRAIGLHDPTAHAEIMALRQGGL
VMQNYRLYDATLYVTFEPCVMCAGAMIHSRIGRVVFGVRNAKTGA
AGSLMDVLHHPGMNHRVEITEGILADECAALLCRFFRMPRRVFNA
QKKAQSSTDGSSGSETPGTSESATPESSGPKKKRKVGTMKEKYIL
GLALGITSVGYGIINFETKKIIDAGVRLFPEANVDNNEGRRSKRG
SRRLKRRRIHRLDRVKSLLTEYNLINREQIPTSNNPYQIRVKGLS
EILSKDELAIALLHLAKRRGIHNINVSSEDEDASNELSTKEQINR
NNKLLKNKYVCEVQLQRLKEGQIRGEKNRFKTTDILKEIDQLLKV
QKDYHNLDIDFINQYKEIVETRREYFEGPGQGSPFGWNGDLKKWY
EMLMGHCTYFPQELRSVKYAYSADLFNALNDLNNLIIQRNDSEKL
EYHEKYHIIENVFKQKKKPTLKQIAKEIGVNPEDIKGYRITKSGT
PQFTEFKLYHDLKSIVFDKSILENEAILDQIAEILTIYQDEESIK
EELNKLPEILNEQDKAEIAKLTGYNVTHRLSLKCIHLINEELWQT
SRNQMEIFNYLNIKPNKVDLSEQNKIPKDLVDEFILSPVVKRTFI
QSINVINKVIEKYGIPEDIIIELARENNSDDRKKFINNLQKKNEA
TRKRINEIIGQTGNQNGKRIVEKIRLHDQQEGKCLYSLESIPLMD
LLNNPQNYEVDHIIPRSVAFDNSIHNKVLVKQIENSKKGNRTPYQ
YLNSSDANLSYNQFKQHILNLSKSKDRISKKKKDYLLEERDINKF
EVQKEFINRNLVDTRYATRELTSYLKAYFSANNMDVKVKTINGSF
TNHLRKVWRFDKYRNHSYKHHAEDALIIANADFLFKENKKLQNAN
KILEKPTIENDTQKVTVEKEEDYNNMFETPKLVEDIKQYRDYKFS
HRVDKKPNRQLIKDTLYSTRMKDEHNYIVQTITDIYGKDNTNLKK
QFNKNPEKFLMYQNDPKTFEKLSIIMKQYSDEKNPLAKYYEETGE
YLTKYSKKNNGPIVKKIKLLGNKVGNHLDVTNKYENSTKKLVKLS
IKNYRFDVYLTEKGYKFVTIAYLNVFKKDNYYYIPKDLYQELKAK
KKIKDTDQFIASFYKNDLIKLNGDLYKIIGVNSDDRNIIELDYYD
IKYKDYCEINNIKGEPRIKKTIGKKTESIEKLTTDVLGNLYLHTT
EKAPQLIFKRGLKRPAATKKAGQAKKKKGSYPYDVPDYAYPYDVP
DYAYPYDVPDYA.
(k)
(SEQ ID NO: 39)
MPAAKRVKLDGSEVEFSHEYWMRHALTLAKRARDEREVPVGAVLV
LNNRVIGEGWNRAIGLHDPTAHAEIMALRQGGLVMQNYRLYDATL
YVTFEPCVMCAGAMIHSRIGRVVFGVRNAKTGAAGSLMDVLHHPG
MNHRVEITEGILADECAALLCRFFRMPRRVFNAQKKAQSSTDGSS
GSETPGTSESATPESSGPKKKRKVGTMKRNYILGLAIGITSVGYG
IIDYETRDVIDAGVRLFKEANVENNEGRRSKRGARRLKRRRRHRL
QRVKKLLFDYNLLTDHSELSGINPYEARVKGLSQKLSEVEFSAAL
LHLAKRRGVHNVNEVEEDTGNELSTKEQISRNSKALEEKYVAELQ
LERLKTDGEVRGPNNRFKTSDYVKEAKQLLKVQKAYHQLDQSFID
TYIDLLETRRTYYEGPGEGSPFGWKDIKEWYEMLMGHCTYFPEEL
RSVKYAYNADLYNALNDLNNLVIARDENEKLEYYEKFQIIENVFK
QKKKPTLKQIAKELLVNEEDIKGYRVTSIGKPEFTNFKIYHDIKG
ITERKEVLENAELLDQIAEILTIYQSSEDVQEELANLNSELTQEE
IEQISNLKGYTGTHNLSLKAINLILDELWHTNDNQMAIFNRLKLV
PKKVDLSQQKEIPTTLVDDFILSPVVKRSFIQSIKVINAIIKKYG
LPNDIIIELAREKNSKDAQKMINEMQKRNRQMNERIEEIIRTTGK
ENAKYLIEKIKLHDMQEGKCLYSLESIPLEDLLNNPFNYEVDHII
PRSVSFDNSFNNKVLVKQEENSKKGNRTPFQYLSSSDSKISYETF
KKHILNLAKGKGRISKTKKEYLLEERDINRFSVQKDFINRNLVDT
RYATRELMNLLRSYFRVNNLDVKVKSINGGFTSFLRRKWKFKKER
NKGYKHHAEDALIIANADFIFKEWKKLDKAKKVMENQTVEEKQAE
SMPEIETEQEYKEIFITPHQIQHIKGFKDYKYSHRVDKKPNRELI
NDTLYSTRKDDKGNTLIVNNLNGLYDKDNDKLKKLMNKSPEKLLM
YHHDPQTYQKLKLIMEQYGDEKNPLYKYYEETGNYLTKYSKKDNG
PVIKKIKYYGKNLKAHLDITDDYPNSRNKVVKLSVKPYRFDVYLD
NDIYKFVTVKNLDVIKKEDYYEVNSKCYKEAKKLKKISDQAEFIA
SFYNNDLIKINGELYRVIGVNNDLLNRIEVNMINITYREYLENMN
DKRPPRIIKTIASKTQSIKKYSTDILGNLYEVKSKQKPQMIMKGK
RPAATKKAGQAKKKKGSYPYDVPDYAYPYDVPDYAYPYDVPDYA.
(l)
(SEQ ID NO: 40)
MPAAKRVKLDGSEVEFSHEYWMRHALTLAKRARDEREVPVGAVLV
LNNRVIGEGWNRAIGLHDPTAHAEIMALRQGGLVMQNYRLYDATL
YVTFEPCVMCAGAMIHSRIGRVVFGVRNAKTGAAGSLMDVLHHPG
MNHRVEITEGILADECAALLCRFFRMPRRVFNAQKKAQSSTDGSS
GSETPGTSESATPESSGPKKKRKVGTMTNGKILGLAIGIASVGVG
IIEAKTGKVVHANSRLFSAANAENNTERRGFRGSRRLNRRKKHRV
KRVRDLFEKHEIVTDFRNLNLSPYELRVKGLTEQLTNEELFAALR
TISKRRGISYLDDAEDDSTGSTDYAKSIDENRRLLKNKTPGQIQL
ERLEKYGQLRGNFTVYDENGEAHRLINVESTSDYEKEARKILETQ
ADYNKKITAEFIDDYVEILTQKRKYYHGPGNEKSRTDYGRFRTDG
TTLENIFGILIGKCNFYPDEYRASKASYTAQEYNFLNDLNNLKVP
TETGKLPTEQKESLVEFAKNTATLGPSKLLKEIAKILDCKVDEIK
GYREDDKGKPDLHTFEPYRKLKFNLESINIDDLSREVIDKLADIL
TLNTEREGIEDAIKRNLPNQFTEEQISEIIKVRKSQSTAFNKGWH
SFSAKLMNELIPELYATSDEQMTILTRLEKFKVNKKSSKNTKTID
EKEVTDEIYNPVVAKSVRQTIKIINAAVKKYGDFDKIVIEMPRDK
NADDEKKFIDKRNKENKKEKDDALKRAAYLYNSSDKLPDEVFHGN
KQLETKIRLWYQQGERCLYSGKPIPIHDLVHNSNNFEIDHILPLS
LSFDDSLANKVLVYDWANQEKGQKTPYQVIDSMDAAWSFREMKDY
VLKQKGLGKKKRDYLLTTENIDKIEVKKKFIERNLIDTRYASRVV
LNSLQSALRELCKDTKVSVIRGQFTSQLRRKWKIDKSRETYHHHA
VDALIIAASSQLKLWEKQDNLMFIDYGNNQVVDKETGEILSVSDD
EYKELVFQPPYQGFVNTISSKGFEDEILFSYQVDSKYNRKVSDAT
IYSTRKAKIGKDKKEETYVLGKIKDIYSQNGFDTFIKKYNKDKTQ
FLMYQKDPLTWENVIEVILRDYPTTKKSEDGKNDVKCNPFEEYRR
ENGLICKYSKKGKGTPIKSLKYYDKKLGNCIDITPEESRNKVILQ
SINPWRADVYFNPETLKYELMGLKYSDLSFEKGTGKYHISQEKYD
AIKEKEGIGKKSEFKFTLYRNDLILIKDTASGEQEIYRFLSRTMP
NVNHYAELKPYDKEKFDGGQELMEVFGKVANGGQCLKSLNKSNIS
IYKVRTDVLGNKYFVKKEGDKPKLNFKNNKKKRPAATKKAGQAKK
KKGSYPYDVPDYAYPYDVPDYAYPYDVPDYA.
(m)
(SEQ ID NO: 41)
MPAAKRVKLDGSEVEFSHEYWMRHALTLAKRARDEREVPVGAVLV
LNNRVIGEGWNRAIGLHDPTAHAEIMALRQGGLVMQNYRLYDATL
YVTFEPCVMCAGAMIHSRIGRVVFGVRNAKTGAAGSLMDVLHHPG
MNHRVEITEGILADECAALLCRFFRMPRRVFNAQKKAQSSTDGSS
GSETPGTSESATPESSGPKKKRKVGTMKRVNEDYILGLAIGTNSC
GWAVTDKKNNLLKLRGKTAIGSHLFEEGHTAADRRGFRTTRRRLK
RRKWRLRLLEEIFAEPMAKVDPGFFVRLHQSWVSPLDKDRKKYNA
IVFPTAKEDQAFYKHYATIYHLRDELMTQDRQFDLREIFLAIHHI
VKYRGNFLQDTPVKDFEASKIEVGPILSHINNAFAEKIVEDQDPI
ELNVANAADIEDVIRGKDAEKTVYKLDKVKKIAKLLTDSTAKEEK
NVAKQIANAIMGYKTQFETILDKEIDKSDKAQWEFKLSDADADDK
LDALLPDLDETDQTVVAEIEKLESAITLSTIVDENKSLSQSMVEK
YKKHKKDYKKLKKYINTLQDQTKAKKLLLAYDLYVNNRHGRLLEA
KKTFEDKKKKKALTKDEFYKIVKDNLDDSDLAHEIQQEIAADNFM
PKQRTNSNGVIPFQLHQIELDKIIANQGKYYPFLAAENPVEDHRK
QAPYKLDELVRFRVPYYVGPMITADEQEKTSGKSFAWMVRKEDGQ
ITPWNFEQKVDRQESANKFIKRMTIKDTYLLSEDVLPANSLLYQR
FEVLNELNNIRINGSRISVDLKQQIFNDLFEEKKTVTEKSLTSYL
KQNLHLPTVEIKGLADPTKFNSSLASYYHLKSLHVFDKELADPQY
QKDFEKIIEYSSIFEDKKIFQDKLHAEFKWLTPEQFKAISTWRLQ
GWGRLSRKLLVELHDTNGQNIMEQLWDSQKNFMQIVTEPDFKDAI
AKENQNVTRANGVEEILADAYTSPANKKAIRQVVKVVADVVKAAG
GKKPAQFAIEFTRDPDKNPQLSHIRGTKLLKAYQETAGELVDQKL
TDSLKEAMTSRKLLKDKYFLYFMQAGRDAYTGQKINIDEVSTNYQ
IDHILPQSFIKDDSFDNRVLTATPLNAEKSDDVPYKRFANNYVSD
MKMTVGEMWKHWQKAGIINKHKLGNLLLDPDRLNKFQKSGFINRQ
LVETSQIIKLVSVILQNKYPDAEIITVKAGDNSALRQRLNLYKSR
DVNDYHHAIDAYLSIICGNFLYQVYPKYRPYFVYGKYKKFSQDPD
LQKEVIKHFKGFTFMWPLLQKDNSERKAPEKIKENNSDRIVFYKH
PDIFDKLRKAYNYKYMLVSRETTTENSGLFDVTIYPRGERDLAKT
RKLIPKSNGLDPKIYGGYSGNTDAYMVIVKIDKGKESIYKVIGVP
MRALASLNRAKKQGNYKEELHQVLEPQIMFDKNGKPKRSVKGFRI
IKDHVPFKQVVLDGDKKFMLNSSTYEINAKQLTLTPETMRIVTDN
LKKGEDQDQLLVKAYDEILQKVDQYLPLFDVNKFRNSLHLGRAKF
LDLAVNDKKITLTNILNGLHDNLVTPDLKNIGIKTPLGKLQVPSG
IVLSSEAILIFQSPTGLFEKRVRIADLKRPAATKKAGQAKKKKGS
YPYDVPDYAYPYDVPDYAYPYDVPDYA.
(n)
(SEQ ID NO: 42)
MPAAKRVKLDGSEVEFSHEYWMRHALTLAKRARDEREVPVGAVLV
LNNRVIGEGWNRAIGLHDPTAHAEIMALRQGGLVMQNYRLYDATL
YVTFEPCVMCAGAMIHSRIGRVVFGVRNAKTGAAGSLMDVLHHPG
MNHRVEITEGILADECAALLCRFFRMPRRVFNAQKKAQSSTDGSS
GSETPGTSESATPESSGPKKKRKVGTMSNGKILGLAIGIASVGVG
VIDAQTGEIIHASSRIFPSANAANNAERRTFRGSRRLIRRKKHRI
KRLDDLFNDFHINLDGEMSTDNPYVLRVKGLSQKLTVEELYISIK
NIMKRRGISYLDDAESDNEAGRSDYAKAIERNRQLLTSKTPGEIQ
LERLEKYGQLRGNFTIIDEEGQSQQIINVESTSDYVKEVEKILDC
QKMYHKFISDEFCDKLIELLREKRKYYVGPGNEKSRTDYGIYRTD
GTTLENLFGILIGKCTFYPDQYRSSRASYTAQEFNFLNDLNNLTV
PTETKKLSQEQKEFLVNYAKETSVLGAGKILQQIAKLADCKVEDI
RGYRLDNKDKPELHTFETYRAMKGLVPLVDIGVLSREQLDILADI
LTLNTDFEGIREALKKQLPNVFDEKQVKGLASFRKSKSQLFAKGW
HNLSQKIMLEVIPELYATSDEQMTILTRLGKFEKSSVAEYPSSIN
VDEITDEIYNPVVAKSIRQTIKIINASIKKWDEFDQIVIEMPRDR
NEDEEKKRIADGQKANAKEKADSILRAAELYCAGKVLPDYVYNGH
NQLATKIRLWYQQGERCIYTGQPISIHDLIHNQNQYEIDHILPLS
LTEDDSLSNKVLVLATANQEKAQRTPYNYLKSATSAWSYREFKDY
VTKRKGIGKKKCEYLTFEEDINGFEVRSKFIQRNLVDTRYASKVI
LNALQDYFKISGIQTKVSVVRGQFTSQLRHKWGIEKTRETYHHHA
VDALIIAASSQLRLWKKQESPLVVDYQEGRQVDLETGEILELTDE
QYKELVYQPPYQGFVNTISSSAFDNEILFSYQVDSKVNRKISDAT
IYATRNAQLGKDKTEGIYVLGKIKDIYTQAGYEAFLKRYTKDKTS
FLMYHKDLDTWEKVIEIILRDYREYDEKGKEIGNPFERYRRENGY
VKKYSRKGNGTAIKSLKYYDNKLGNHIDITPENSRNAVVLQSLKP
WRTDVYFNKETGKYEFLGIKYSDLSFEKGTGEYGISQEKYDSIKI
AEGVAKKSIFKFTLYKQDLLFIKDIENNFGKLLRFTSKNDTSKHY
VELKPYDKNKFGTEEPLLPVLGNVAKSGQCIKGLNKSNISIYKVR
TDILGYRHFIKQEGEHPQLKFKKKRPAATKKAGQAKKKKGSYPYD
VPDYAYPYDVPDYAYPYDVPDYA.

40. The Cas9 protein of claim 38, wherein the Cas9 protein is fused to a cytosine deaminase.

41. The Cas9 protein of claim 2, wherein the Cas9 protein recognizes a PAM sequence comprising 5′-NRGNR-3′.

42. The Cas9 protein of claim 3, wherein the Cas9 protein recognizes a PAM sequence comprising 5′-NRG-3′, wherein H is adenine, cytosine, or thymine.

43. The Cas9 protein of claim 4, 11 or 12, wherein the Cas9 protein recognizes a PAM sequence comprising 5′-NNGR-3′, wherein H is adenine, cytosine or thymine, and R is adenine or guanine.

44. The Cas9 protein of claim 5, wherein the Cas9 protein recognizes a PAM sequence comprising 5′-NNGRRT-3′.

45. The Cas9 protein of claim 6, wherein the Cas9 protein recognizes a PAM sequence comprising 5′-NNAAAA-3′.

46. The Cas9 protein of claim 7, wherein the Cas9 protein recognizes a PAM sequence comprising 5′-NGGNG-3′.

47. The Cas9 protein of claim 8, wherein the Cas9 protein recognizes a PAM sequence comprising 5′-NNGNRG-3′.

48. The Cas9 protein of claim 9, wherein the Cas9 protein recognizes a PAM sequence comprising 5′-NNAAAC-3′.

49. The Cas9 protein of claim 10, wherein the Cas9 protein recognizes a PAM sequence comprising 5′-NNRAAG-3′.

50. The Cas9 protein of claim 13, wherein the Cas9 protein recognizes a PAM sequence comprising 5′-NNAYAA-3′.

51. The Cas9 protein of claim 13, wherein the Cas9 protein recognizes a PAM sequence comprising 5′-NNGAAA-3′.

52. The Cas9 protein of claim 13, wherein the Cas9 protein recognizes a PAM sequence comprising 5′-NNAAA-3′. (H=A, C or T; R=A or G).

53. A nucleic acid encoding the Cas9 protein of any one of the preceding claims.

54. The nucleic acid of claim 53, wherein the nucleic acid is codon-optimized for expression in mammalian cells.

55. The nucleic acid of claim 54, wherein the nucleic acid is codon-optimized for expression in human cells.

56. A eukaryotic cell comprising the Cas9 protein of any one of claims 1-52.

57. The eukaryotic cell of claim 55, wherein the cell is a human cell.

58. A method of cleaving a target nucleic acid in a eukaryotic cell comprising:
contacting the cell with a Cas9 of any one of claims 1-52, and an RNA guide or a nucleic acid encoding the RNA guide, wherein the RNA guide comprises a direct repeat sequence and a spacer sequence capable of hybridizing to the target nucleic acid, and
wherein the Cas9 protein is capable of binding to the RNA guide and of causing a break in the target nucleic acid sequence complementary to the RNA guide.

59. A method of altering expression of a target nucleic acid in a eukaryotic cell comprising:
contacting the cell with a Cas9 of any one of claims 1-52, and an RNA guide or a nucleic acid encoding the RNA guide, wherein the RNA guide comprises a direct repeat sequence and a spacer sequence capable of hybridizing to the target nucleic acid, and
wherein the Cas9 protein is capable of binding to the RNA guide and of causing a break in the target nucleic acid sequence complementary to the RNA guide.

60. A method of altering expression of a target nucleic acid in a eukaryotic cell comprising:
contacting the cell with a Cas9 of any one of claims 1-52, and an RNA guide or a nucleic acid encoding the RNA guide, wherein the RNA guide comprises a direct repeat sequence and a spacer sequence capable of hybridizing to the target nucleic acid, and
wherein the Cas9 protein is capable of binding to the RNA guide and editing the target nucleic acid sequence complementary to the RNA guide.

61. A method of modifying a target nucleic acid in a eukaryotic cell comprising:
contacting the cell with a Cas9 of any one of claims 1-52, and an RNA guide or a nucleic acid encoding the RNA guide, wherein the RNA guide comprises a direct repeat sequence and a spacer sequence capable of hybridizing to the target nucleic acid, and
wherein the Cas9 protein is capable of binding to the RNA guide and editing the target nucleic acid sequence complementary to the RNA guide.

62. The method of claim 60 or 61, wherein the Cas9 protein is an inactive Cas9 (dCas9).

63. The method of claim 62, wherein the dCas9 is fused to a deaminase.

64. The method of any one of claims 58-63, wherein the RNA guide comprises a crRNA and a tracrRNA.

65. The method of any one of claims 58-64, wherein the RNA guide comprises a sgRNA.

66. The method of claim 65, wherein the sgRNA for use with Seq2Cas9 comprises a scaffold comprising a sequence having at least about 80% identity to 5′-GUUUUAGUACUCUGUAAUUUUGAAAAAAAUUACAGAAUCUACUAAAACAAGG CAAAAUGCCGUGUUUAUCUCGUCAACUUGUUGACGAGAUUUUUUU-3′ (SEQ ID NO: 43).

67. The method of claim 65, wherein the sgRNA for use with EhiCas9 comprises a scaffold comprising a sequence having at least about 80% identity to 5′-GUUUUAGAGCUAUGUUGGAAACAACAUAGCGAGUUAAAAUAAGGCAUUGUCC GUUAUCAGCUUUUAAAGCAAGCACUGUCUCGGUGCUUUUUU-3′ (SEQ ID NO:
44).

68. The method of claim 65, wherein the sgRNA for use with SeqCas9 comprises a scaffold comprising a sequence having at least about 80% identity to 5′-GUUUUUGUACUCUCAAGAUUUGAAAAAAUCUUGCAGAGCCUACAAAGAUAAG GCUUCAUGCCGAAUUCAAGCACCCCAUGUUUACAUGGGGUGCUUUU-3′ (SEQ ID NO: 45).

69. The method of claim 65, wherein the sgRNA for use with SsiCas9 comprises a scaffold comprising a sequence having at least about 80% identity to 5′-GUUUUAGUACUCUGUAAUUUUGAAAAAAAUUACAGAAUCUACUAAAACAAGG CAAAAUGCCGUGUUUAUCUCGUCAACUUGUUUGGCGAGAUUUUUUUU-3′ (SEQ ID NO: 46).

70. The method of claim 65, wherein the sgRNA for use with SinCas9 comprises a scaffold comprising a sequence having at least about 80% identity to 5′-GUUUUUGUACUCUCAAGAUUUGAAAAAAUCUUGCAGAAGCUACAAAGAUAAG GCUUCAUGCCGAAAUCAACACCCUGUCUAUGACGGGGUGUUUU-3′ (SEQ ID NO: 47).

71. The method of claim 65, wherein the sgRNA for use with SsaCas9 comprises a scaffold comprising a sequence having at least about 80% identity to 5′-GUUUUAGAGCUGUGUUGUGAAAACAACACAGCAAGUUAAAAUAAGGCUUUGU 15 CCGUACACAACUUGAAAAAGUGCGCACCGAUUCGGUGCUUUUUU-3′ (SEQ ID NO: 48).

72. The method of claim 65, wherein the sgRNA for use with Ssc2Cas9 comprises a scaffold comprising a sequence having at least about 80% identity to 5′-GUUUUUGUACUCUCAAGAUUUGAAAAAAUCUUGCAGAAGCUACAAAGAUAAG GCUUCAUGCCGAAAUCAACACCCUGUCAUUUUAUGGCAGGGUGUUUU-3′ (SEQ ID NO: 49).

73. The method of claim 65, wherein the sgRNA for use with Sor2Cas9 comprises a scaffold comprising a sequence having at least about 80% identity to 5′-GUUUUUGUACUCUCAAGAUUUGAAAAAAUCUUGCAGAAGCUACAAAGAUAAG GCUUCAUGCCGAAUUCAACACCCUGUCAUUUAUGGCGGGGUGUUUU-3′ (SEQ ID NO: 50).

74. The method of claim 65, wherein the sgRNA for use with SorCas9 comprises a scaffold comprising a sequence having at least about 80% identity to 5′-GUUUUUGUACUCUCAAGAUUUGAAAAAAUCUUGUAGAAGCUACAAAGAUAAG GCUUCAUGCCGAAUUCAACACCCUGUCAUUUAUGGCGGGGUGUUUCGUUUU-3′ (SEQ ID NO: 51).

75. The method of claim 65, wherein the sgRNA for use with SwaCas9 comprises a scaffold comprising a sequence having at least about 80% identity to 5′-GUUUUAGUACUCUGUAAUUUUGAAAAAAAUUACAGAAUCUACUGAAACAAGA CUAUAUGUCGUGUUUAUCCCACUAAUUUAUUAGUGGGAUUUUUU-3′ (SEQ ID NO: 52).

76. The method of claim 65, wherein the sgRNA for use with SscCas9 comprises a scaffold comprising a sequence having at least about 80% identity to 5′-GUUUUAGUACUCUGUAAUUUUGAAAAAAAUUACAGAAUCUACUAAAACAAGG CAAAAUGCCGUGUUUAUCUCGUCAACUUGUUGACGAGAUUUUUUU-3′ (SEQ ID NO: 53).

77. The method of claim 65, wherein the sgRNA for use with SgaCas9 comprises a scaffold comprising a sequence having at least about 80% identity to 5′-GUUUUUGUACUCUCAAGAUUUGAAAAAAUCUUGCUGAGCCUACAAAGAUAAG GCUUUAUGCCGAAUUCAAGCACCCCAUGUUUUGACAUGGGGUGCUUUU-3′ (SEQ ID NO: 54).

78. The method of claim 65, wherein the sgRNA for use with LkuCas9 comprises a scaffold comprising a sequence having at least about 80% identity to 5′-GUUUUAGAUGAUUGUUAGAUCGAAAGAUCUAACAACCAGAUUUUAAAAUCAA ACAAUGUAUCUUUGAUACUAAGUUUCAACGCGGUAUUAUUACCGUCCUGCCUC AGCUCUAUAGCGGAGGUUUUUU-3′ (SEQ ID NO: 55).

79. The method of claim 65, wherein the sgRNA for use with SsuCas9 comprises a scaffold comprising a sequence having at least about 80% identity to 5′-GUUUUUGUACUCUCAAGAUUUGAAAAAAUCUUGCAGAGCCUACAAAGAUAAG GCUUUAUGCCGAAAUCAAGCACCCCGUUUCGUACGGGGUGCUUUU-3′ (SEQ ID NO: 56).

80. The method of claim 64, wherein the crRNA comprises a guide sequence of between about 16 and 26 nucleotides long.

81. The method of claim 64, wherein the crRNA comprises a guide sequence between 18 and 24 nucleotides long.

82. The method of claim 80 or 81, wherein the break in the target nucleic acid is a single-stranded or double-stranded break.

83. The method of claim 82, wherein the break in the target nucleic acid is a single-stranded break.

84. The method of claim 60 or 61, wherein the Cas9 protein is a nuclease that cleaves both strands of the target nucleic acid sequence, or is a nickase that cleaves one strand of the target nucleic acid sequence.

85. The method of any one of claims 58-84, wherein the target nucleic acid is 5′ to a protospacer adjacent motif (PAM) sequence.

86. The method of any one of claims 58-85, wherein the Cas9 is operably linked to a promoter sequence for expression in a eukaryotic cell, and wherein the guide RNA is operably linked to a promoter sequence for expression in a eukaryotic cell.

87. The method of claim 86, wherein the eukaryotic cell is a human cell.

88. The method of claim 86, wherein the promoter sequence is a eukaryotic or viral promoter.

89. An engineered, non-naturally occurring CRISPR-Cas system comprising:
an RNA guide or a nucleic acid encoding the RNA guide, wherein the RNA guide comprises a direct repeat sequence and a spacer sequence capable of hybridizing to a target nucleic acid; and
a CRISPR-associated (Cas) protein having at least 80% sequence identity to SEQ ID NOs: 1, 2, 3, 4, 5, 6, 7, 8, 9, 10, 11, 12, 13 or 14, and wherein the Cas protein is capable of binding to the RNA guide and of causing a break in the target nucleic acid sequence complementary to the RNA guide.

90. An engineered, non-naturally occurring CRISPR-Cas system comprising:
an RNA guide or a nucleic acid encoding the RNA guide, wherein the RNA guide comprises a direct repeat sequence and a spacer sequence capable of hybridizing to a target nucleic acid; and
a CRISPR-associated (Cas) protein having at least 80% sequence identity to SEQ ID NOs: 1, 2, 3, 4, 5, 6, 7, 8, 9, 10, 11, 12, 13 or 14;
wherein the Cas protein is fused to a deaminase, and wherein the Cas protein fusion is capable of binding to the RNA guide and of editing the target nucleic acid sequence complementary to the RNA guide.

91. The system of claim 90, wherein the Cas9 protein is an inactive Cas9 (dCas9).

92. The system of claim of any one of claims 90-91, wherein the RNA guide comprises a crRNA and a tracrRNA.

93. The system of any one of claims 90-92, wherein the RNA guide comprises an sgRNA.

94. The system of claim 93, wherein the sgRNA for use with Seq2Cas9 comprises a scaffold comprising a sequence having at least about 80% identity to 5′-GUUUUAGUACUCUGUAAUUUUGAAAAAAAUUACAGAAUCUACUAAAACAAGG CAAAAUGCCGUGUUUAUCUCGUCAACUUGUUGACGAGAUUUUUUU-3′ (SEQ ID NO: 43).

95. The system of claim 93, the sgRNA for use with EhiCas9 comprises a scaffold comprising a sequence having at least about 80% identity to 5′-GUUUUAGAGCUAUGUUGGAAACAACAUAGCGAGUUAAAAUAAGGCAUUGUCC GUUAUCAGCUUUUAAAGCAAGCACUGUCUCGGUGCUUUUUU-3′ (SEQ ID NO: 44).

96. The system of claim 93, wherein the sgRNA for use with SeqCas9 comprises a scaffold comprising a sequence having at least about 80% identity to 5′-GUUUUUGUACUCUCAAGAUUUGAAAAAAUCUUGCAGAGCCUACAAAGAUAAG GCUUCAUGCCGAAUUCAAGCACCCCAUGUUUACAUGGGGUGCUUUU-3′ (SEQ ID NO: 45).

97. The system of claim 93, wherein the sgRNA for use with SsiCas9 comprises a scaffold comprising a sequence having at least about 80% identity to 5′-GUUUUAGUACUCUGUAAUUUUGAAAAAAAUUACAGAAUCUACUAAAACAAGG CAAAAUGCCGUGUUUAUCUCGUCAACUUGUUUGGCGAGAUUUUUUUU-3′ (SEQ ID NO: 46).

98. The system of claim 93, wherein the sgRNA for use with SinCas9 comprises a scaffold comprising a sequence having at least about 80% identity to 5′-GUUUUUGUACUCUCAAGAUUUGAAAAAAUCUUGCAGAAGCUACAAAGAUAAG GCUUCAUGCCGAAAUCAACACCCUGUCUAUGACGGGGUGUUUU-3′ (SEQ ID NO: 47).

99. The system of claim 93, wherein the sgRNA for use with SsaCas9 comprises a scaffold comprising a sequence having at least about 80% identity to 5′-GUUUUAGAGCUGUGUUGUGAAAACAACACAGCAAGUUAAAAUAAGGCUUUGU 20 CCGUACACAACUUGAAAAAGUGCGCACCGAUUCGGUGCUUUUUU-3′ (SEQ ID NO: 48).

100. The system of claim 93, wherein the sgRNA for use with Sor2Cas9 comprises a scaffold comprising a sequence having at least about 80% identity to 5′-GUUUUUGUACUCUCAAGAUUUGAAAAAAUCUUGCAGAAGCUACAAAGAUAAG GCUUCAUGCCGAAAUCAACACCCUGUCAUUUUAUGGCAGGGUGUUUU-3′ (SEQ ID NO: 49).

101. The system of claim 93, wherein the sgRNA for use with Ssc2Cas9 comprises a scaffold comprising a sequence having at least about 80% identity to 5′-GUUUUUGUACUCUCAAGAUUUGAAAAAAUCUUGCAGAAGCUACAAAGAUAAG GCUUCAUGCCGAAAUCAACACCCUGUCAUUUUAUGGCAGGGUGUUUU-3′ (SEQ ID NO: 50).

102. The system of claim 93, wherein the sgRNA for use with Sor2Cas9 comprises a scaffold comprising a sequence having at least about 80% identity to 5′-GUUUUUGUACUCUCAAGAUUUGAAAAAAUCUUGCAGAAGCUACAAAGAUAAG 5 GCUUCAUGCCGAAUUCAACACCCUGUCAUUUAUGGCGGGGUGUUUU-3′ (SEQ ID NO: 51).

103. The system of claim 93, wherein the sgRNA for use with SwaCas9 comprises a scaffold comprising a sequence having at least about 80% identity to 5′-GUUUUAGUACUCUGUAAUUUUGAAAAAAAUUACAGAAUCUACUGAAACAAGA CUAUAUGUCGUGUUUAUCCCACUAAUUUAUUAGUGGGAUUUUUU-3′ (SEQ ID NO: 52).

104. The system of claim 93, wherein the sgRNA for use with SscCas9 comprises a scaffold comprising a sequence having at least about 80% identity to 5′-GUUUUAGUACUCUGUAAUUUUGAAAAAAAUUACAGAAUCUACUAAAACAAGG CAAAAUGCCGUGUUUAUCUCGUCAACUUGUUGACGAGAUUUUUUU-3′ (SEQ ID NO: 53).

105. The system of claim 93, wherein the sgRNA for use with SgaCas9 comprises a scaffold comprising a sequence having at least about 80% identity to 5′-GUUUUUGUACUCUCAAGAUUUGAAAAAAUCUUGCUGAGCCUACAAAGAUAAG GCUUUAUGCCGAAUUCAAGCACCCCAUGUUUUGACAUGGGGUGCUUUU-3′ (SEQ ID NO: 54).

106. The system of claim 93, wherein the sgRNA for use with LkuCas9 comprises a scaffold comprising a sequence having at least about 80% identity to 5′-GUUUUAGAUGAUUGUUAGAUCGAAAGAUCUAACAACCAGAUUUUAAAAUCAA ACAAUGUAUCUUUGAUACUAAGUUUCAACGCGGUAUUAUUACCGUCCUGCCUC AGCUCUAUAGCGGAGGUUUUUU-3′ (SEQ ID NO: 55).

107. The system of claim 93, wherein the sgRNA for use with SsuCas9 comprises a scaffold comprising a sequence having at least about 80% identity to 5′-GUUUUUGUACUCUCAAGAUUUGAAAAAAUCUUGCAGAGCCUACAAAGAUAAG GCUUUAUGCCGAAAUCAAGCACCCCGUUUCGUACGGGGUGCUUUU-3′ (SEQ ID NO: 56).

108. The system of any one of claims 90-107, wherein the nucleic acid encoding the Cas protein is operably linked to a promoter sequence for expression in a eukaryotic cell, and wherein the guide RNA is operably linked to a promoter sequence for expression in a eukaryotic cell.

109. The system of claim 108, wherein the eukaryotic cell is a human cell.

110. The system of claim 108, wherein the promoter sequence is a eukaryotic promoter sequence.

111. A nucleic acid encoding the system of any one of claims 90-110.

112. A vector comprising the system of any one of claims 90-110.

113. The vector of claim 112, wherein the vector is a plasmid vector or a viral vector.

114. The vector of claim 113, wherein the viral vector is an adeno associated virus (AAV) vector or a lentiviral vector.

115. The vector of claim 114, wherein the viral vector is an AAV vector.

116. The vector of claim 115, wherein more than one AAV vector is used for packaging the system of claims 90-110.

118. A method of treating a disorder or a disease in a subject in need thereof, the method comprising administering to the subject a system of any one of claims 90-111.
wherein the guide RNA is complementary to at least 10 nucleotides of a target nucleic acid associated with the condition or disease;
wherein the Cas protein associates with the guide RNA;
wherein the guide RNA binds to the target nucleic acid;
wherein the Cas protein causes a break in the target nucleic acid, optionally wherein the Cas9 is an inactive Cas9 (dCas9) fused to a deaminase and results in one or more base edits in the target nucleic acid, thereby treating the disorder or disease.

119. The method of claim 118, wherein the guide RNA is complementary to about 18-24 nucleotides.

120. The method of claim 118, wherein the guide RNA is complementary to 20 nucleotides.

121. A base editor comprising the fusion protein of any one of claims 37-40.

122. The base editor of claim 118 comprising an adenosine deaminase domain or a cytidine deaminase domain.

123. A method of editing a nucleobase of a polynucleotide, the method comprising contacting the polynucleotide with the base editor of claim 118 in complex with one or more guide RNAs, wherein the base editor comprises an adenosine deaminase domain and wherein the one or more guide RNAs target the base editor to effect an A⋅T to G⋅C alteration in the polynucleotide.

124. A method of editing a nucleobase of a polynucleotide, the method comprising contacting the polynucleotide with the base editor of claim 118 in complex with one or more guide RNAs, wherein the base editor comprises a cytidine deaminase domain, and wherein the one or more guide RNAs target the base editor to effect an C⋅G to T⋅A alteration in the polynucleotide.

125. The method of claim 123 or 124, wherein the editing results in less than 50% indel formation in the target polynucleotide sequence.

126. The method of any one of claims 123-125, wherein the editing generates a point mutation.


SEQUENCE LISTING (198 sequences)

SEQ ID NO: 1 - protein, 1398 aa, Streptococcus equinus
MEKSYSIGLDIGTNSVGWSVITDDYKVPAKKMRVLGNTDKKYIKKNLLGALLFDSGETAEATRLKRTARRRYTRRKNRLRYLQEIFAKEMAKVDESFFQRLDESFLTDDDKTFDSHPIFGNKAEEDAYHQKFPTIYHLRKHLADSQEKADLRLIYLALAHMIKFRGHFLYDNFNDDNFDWRNIDIQKRYEEFIETYDSTLGESYLADISVDAASILEEKVSKTERLENLLKYYPTEKKTTFFGNLIKLILGQQAKFKAIFNLEDEISLQFATPTYDEDLEELLGKIDNGDSYSELFVAAQNLYNTILLASFLKTDNKSAKAPLSTSMIERYENHKKDLAKLKDFVKKNCPDQYHDIFRDKSKNGYAGYIDNGVSQNDFYTFIGKCLEESLKKDKGAQYFLDKIDRDDFLRKQRTFDNGAFPYQIHLQEMHAILRRQGDYYPFLKENQDKIEKILTFRIPYYVGPLARKDSRFAWANYRSDEAITPWNFDEVVDKEKSAEKFITRMTLNDLYLPEEKVLPKHSLIYETFTVYNELTNIKYVNDQGNAIHFDSELKEKIFNQLFKENRKVSKKTLIDFLNNSEGIYTDKLVGIDEEVKYLNASLGTYHDLKKILESFMDDEINEKIIEDIIQTLTLFEDIEMKRQRLQKYDDIFTPKQLKELARRNYTGWGRLSYKLINGIRNKENNKTILDYLKNGNRNFMQLINDDRLSFKQIIIDARKIEKLDNIESVVYNLPGSPAIKKGILQSIKIVDELVKVMGHNPDNIVIEMARENQTTNQGKNRSQQRLKRLQDSMSNFKDSSISLKDVDNSDLQNDRLFLYYIQNGKDMYTGEELDIDHLSDYDIDHIIPQSFIKDNSIDNRVLTSSAKNRGKSDDVPGRDVVLKMKPFWKKLYDVKLISKRKFDNLTKSEHGGLTESDKAGFIKRQLVETRQITKYVAQILDGRFNTKRDDNNKVIRDVKVITLKSSLVSQFRKDFGFYKVREINDYHHAHDAYLNAVVGTAILKKYPKLAPEFVYGEYKKCDVRKLIAKSGDKSEIGKATAKYFFYSNLMNFFKRVIRYSNGMIVVRPVIEYSKDTGEIAWDKEKDFKTVCKVLSCPQVNIVKKVEKQSHGLDRGKPKGFYNANPSPKPKKGSKVNLVPIKANLNPKNYGGYAGISNSYAVLVDATIEKGAKKKLTRIQEFQGISIIDREKYEKNKVEFLKGLGYKEIYSIITLPKYSLFELADGSRRMLASILSTNNKRGEIHKGNELVLPAKYIPLLYHANRIHNTFETGHREYVEKHIAEFKEIAEIILEFNNKYVNAKKNSSIIEKALESFDSFSLDEICDSFVGKLKKNNTKKNSGLFELVSLGSASDFEFLETKVPRYRDYTPSSLLNATLIHQSITGLYETRIDLSKLGEE

SEQ ID NO: 2 - protein, 1336 aa, Enterococcus hirae
MTKDYTIGLDIGTNSVGWAVLTDDYQLMKRKMSVHGNTEKKKIKKNFWGARLFDEGQTAEFRRTKRTNRRRLARRKYRLSKLQDLFAEELCKQDDCFFVRLEESFLVPEEKQYKPASIFPTLEEEKEYYQKYPTIYHLRQKLVDSTEKEDLRLVYLALAHLLKYRGHFLFEGDLDTENTSIEESFRVFLEQYSKQSDQPLIVHQPVLTILTDKLSKTKKVEEILKYYPTEKINSFFAQCLKLIVGNQANFKRIFDLEAEVKLQFSKETYEEDLESLLEKIGDEYLDIFLQAKKVHDAILLSEIISSTVKHTKAKLSSGMVERYERHKADLAKFKQFVKENVPQKSTAFFKDTTKNGYAGYIKGKTTQEEFYKFVKKELSGVVGSEPFLEKIDQETFLLKQRTYTNGVIPHQVHLIELKAIIDQQKQHYPFLEEAGPKIIALFKFRIPYYVGPLAKEQEASSFAWIERKTAEKIHPWNFSEVVDIEKSAMRFIQRMTKQDTYLPTEKVLPKNSLLYQKYMIFNELTKVSYKDERGVKQYFSGDEKQQIFKQLFQKERGKITVKKLQNFLYTHYHIENAQIFGIEKAFNASYSTYHDFMKLAKTNQKAMQEWLEQPEMEPIFEDIVKILTIFEDRQMIKHQLSKYQEVFGEKLLKEFARKHYTGWGRFSAKLIHGIRDRKTNKTILDYLINDDDVPANRNRNLMQLINDEHLSFKEEIAKATVFSKHKSLVDVIQDLPGSPAIKKGIWQSLKIVEELIAIIGYKPKNIVIEMARENQKTHRTKPRLKALENGLKQIGSTLLKEQPTDNKALQKERLYLYYLQNGRDMYTGEPLEIENLHQYEVDHIIPRSFIVDNSIDNKVLVARKQNQKKRDDVPKKQIVNEQRIFWNQLKEAKLISPKKYAYLTKIELTPEDKARFIQRQLVETRQITKHVANILHQSFHQEEEGTDCDGVQIITLKATLTSQFRQTFGLYKVREINPHHHAHDAYLNGFIANVLLKRYPKLAPEFVYGKYVKYSLARENKATAKKEFYSNILKFFESDEPFCDENGEIYWEKSHHLPRIKKVLSSHQVNVVKKVEQQKGGFYKETVNSKEKPDKLIERKNNWDVTKYGGFGSPVIAYAIAFVYAKGKTQKKTRAIEGITIMEQAAFEKDPTTFLKEKGFPQVTEFIKLPKYTLFEFDNGRRRFLASHKESQKGNPFILSDQLVTLLYHAQHYDKITYQESFDYVNTHLSDFSAILTEVLAFAEKYTLADKNIERIQELYEENKYGEISMIAQSFLQLLQFNAIGAPADFKFFGVTIPRKRYTSLTEIWDATIIYQSVTGLYETRIRMGDLWAGEQ

SEQ ID NO: 3 - protein, 1130 aa, Streptococcus equinus
MTNGKILGLDIGVASVGVGIIEAKTGKVIHANSRLFSAANAENNAERRGFRGARRLTRRKKHRVKRVRDLFEKYDISTDFRNLNLNPYELRVKGLSEQLTNEELFAALRTIAKRRGISYLDDAEDDSTGSSDYAKSIDENRRLLKSMTPGQIQLERLEKYGQLRGNFTVYDENGEAHRLINVFSTSDYKNEARKILETQSNYNKQITDEFIEDYIEILTQKRKYYHGPGNEKSRTDYGRFRTDGTTLENIFGILIGKCSFYPEEYRASKASYTAQEFNFLNDLNNLKVPTETGKLSTEQKEYLVDFAKNSKTLGASKLLKEIAKLVDGDVKDISGYREDSKGKPDLHTFEPYRKLKFNLTTVDIDNLSRDILDKLANILTLNTEREGIEDAINRNLPEQFTKEQISEIVQIRKSQSSAFNKGWHSFSAKLMNELIPELYATSEEQMTILTRLEKFKATKKSSKNTKTIDEKEITDEIYNPVVAKSVRQTIKIINAAVKKYGDFDKIVIEMPRDKNADDEKKFIDKKNKENKKEKDDSLKRAAYLYNGTDKLPDDVFHGNKQLATKIRLWYQQGERCLYSGKPILIQDLVHNSNNFEIDHILPLSLSFDDSLANKVLVYAWTNQEKGQKTPYQVIDSMDAAWSFREMKDYVLKQKGIGKKKREYLLTTENIDKIEVKKKFIERNLVDTRYASRVVLNSLQTALKELGKDTKVSVVRGQFTSQLRRKWKIDKSRETYHHHAVDALIIAASSQLKLWQKHENLMFENYGENQVVNKETGEILSISDDEYKELVFQPPYQGFVNTISSKAFEDEILFSYQVDSKFNRKVSDATIYSTRKAKLGKDKKEETYVLGKIKDIYSQDGFDTFIKRYKKDKTQFLMYQKDPLTWENVIEVILRDYPTTKKSEDGKNDVKCNPFEEYRRENGLICKYSKKGKGTPIKSLKYYDKKLGNHISITPKESKNDVVLQSLNPWRADLYFNPDTLKYELMGLKYSDLSFEKGTGKYHISQEKYDEIKEKEGIGQNSEFKFTLYRNDLILIKDTESGEQEIYRFLSRTMPNVKHYVELKPYDKEKFNGGQELIKSLGEADKVGRCLKGLSKPGISIYKVRTDVLGNKFFVKKEGDKPKLDFKNNKK

SEQ ID NO: 4 - protein, 1053 aa, Staphylococcus simulans
MNNSYILGLDIGITSVGYGIIEYETRDVIDAGVRLFKEANVENNEGRRSKRGARRLKRRRRHRLQRVKKMLFDYKLLNEDSEISGINPYEARVKGLSEKLSDEEFSAALLHLAKRRGVHNVSDVEEDTGNELSTKEQIARNNKALEDKYVAELQLERLKEQGEVRGAANRFKTSDYIKEAKQLLKTQSDYHKIDETFIETYISLLETRRTYYEGPGEGSPFGWKDIKEWYEMLMGHCTYFPEELRSVKYAYNADLYNALNDLNNLVIARDENEKLEYYEKFQIIENVFKQKKKPTLKQIAKEILVNEEDIKGYRVTSTGKPEFTNFKIYHDIKGITSRKEILENADLLNQIAEILTIYQSAEDIQEELAELEPKLTQEEIEQISNLTGYTGTHRLSLKAINLILDELWNTSDNQMTIFNRLKLVPKKVDLSQQKEIPTSLVDDFILSPVVKRSFIQSIKVINAIIKKFGLPKDIIIELAREKNSKEAQKFINEMQKRNRQTNERIEKIIKETGKEKAKFLIEKIKLHDMQEGKCLYSLESIPLEDLLNNPYHYEVDHIIPRSVSFDNSFNNKVLVKQEENSKKGNRTPFQYLSSSDAKISYETFKKHILNLSKGKGRVSKKKKEYLLEERDINRFSVQKDFINRNLVDTRYATRELMNLLRSYFRVNDLDVKVKSINGGFTSFLRTKWKFKKERNQGYKHHAEDALVIANADFIFKEWKKLDTTNKVMENQTVEEQQAENMPGIETDDEYKEIFVIPRQIQSIKDFKDYKYSHRVDKKPNRELVNDTLYSTRKDDKGNTLIINNIKGLYDKDNDKLKNLIKKSPEKLLMYHHDPQTYQKLKTIMEQYSNEKNPLYKYHEETGNYLTKYSKKDNGPIIKKVKYYGKKLNAHLDITNDYSNSQNKIVKLSLKPYRFDVYLDNGGYKFVTVKNLDVIKKEGFFKIDSNAYEKAKSEKKIDENAVFIASFYNNDLIKIDGELYRIVGVNNDTRNVVELNMIPITYKEYLENINDKRTPRILKTISQKTYSIEKYSTDILGNLYKVKSKKKPQMIMKG

SEQ ID NO: 5 - protein, 1125 aa, Streptococcus intermedius
MNGLVLGLDIGIASVGVGILNKETGEIIHVNSRIFPAATADSNVERRGFRQGRRLGRRKKHRSARLNDLFEEFGFITDFSAVPLNLNPYALRVKGLSEELTNEELFIALKNIIKRRGISYLDDASEDGETASNEYGKAVEENRKLLADKTPGQIQLERFEKYGQVRGDFTVVENGENHRLINVFSTSAYKKEAERILRRQQEFNVRISDEFIEAYLTILTGKRKYYHGPGNEKSRTDYGRFRTDGTTLDNIFGILIGKCTFYPDEYRAAKASYTAQEFNLLNDLNNLTVPTETKKLRPEQKRQIVEYARTAKTLGTPTLLKYIAKLVDGSIDDIKGYRIDKSDKPEMHTFDAYRKMRTLELVDVDILSRETLDDLAYILTLNTESEGILEALNSKMPGTFTKEQIDELIQFRKKNSAVFGKGWHNFSLKLMNELISELYETSEEQMTILTRLGKQRSREISKRTKYIDEKELTDEIYNPVVAKSVRQAIKIINLATKKYGIFDNIVIEMARESNEDDEKKAIQNVQKANEDEKKAAMEKAADLYNGKKELPDSIFHGHKELATKIRLWHQQGERCLYTGKNISIHDLIHNPHQYEIDHILPLSLSFDDGLANKVLVLATANQEKGQRTPFQALDSMDDAWSYIEFKQYVRNSKSLSNKKKDYLLTEEDISKIEVKQKFIERNLVDTRYSSRVVLNTLQEFYKTNDFDTKISVVRGQFTSQLRRKWKIEKSRDTYHHHAVDALIIAASSQLRLWKKQNNPLISYREGQLVDPETGEILSLTDDEYKELVFRPPYDYFVDTLKSKSFEDSILFSYQVDSKYNRKISDATIYGTRKAQLGKDKQEETYVLGKIKDIYSQKGYEDFIKRYKKDTTQFLMYHKDPQTFAKVIEEILKTYPDKELNEKGKEIPCNPFEKYRQENGPIRKYSKKGKGPEIKSLKYYDNKLGNHIDITPVNSQNQVVLQSLKPWRTDVYFNPQTSKYELMGLKYSDLRFEKGSGSYGISPEKYNKVKAKEGVDEDSEFKFTLYKNDLILIKDTETGEQQLFRYGSRNDTSKHYVELKPYEKAKFEGNQQLMNLLGTVAKGGQCLKGINKPNLSIYKVKTDVLGNKHFIKKEGDQPQLNFKKKI

SEQ ID NO: 6 - protein, 1392 aa, Streptococcus sanguinis
MENKNYSIGLDIGTNSVGWAVITDDYKVPSKKMKVFGNTDKHFIKKNLIGALLFDEGATAEDRRLKRTARRRYTRRKNRLRYLQEIFSEEISKLDSSFFHRLDDSFLVPKDKRGSKYPIFATLEEEKEYHKKFPTIYHLRKHLADSKEKTDLRLIYLALAHMIKYRGHFLYEESFDIKNNDIQKIFNEFISIYDNTFEGSSLSGQNAQVEAIFTDKISKSAKRERVLKLFSDEKSTSLFSEFLKLIVGNQADFKKHFDLEEKAPLQFSKDTYDEDLENLLGQIGDGFTDLFLVAKKLYDAILLSGILTVTDPSTKAPLSASMIERYESHQKDLAALKQFIKNNLPKRYNEVFSDQSKDGYAGYIDGKTTQEAFYKYIKNLLSKFEGADYFLDKIEREDFLRKQRTFDNGSIPHQIHLQEMNAILRRQGEHYPFLKENREKIEKILTFRIPYYVGPLARGNRDFAWLTRNSDQAIRPWNFEEIVDKASSAEEFINKMTNYDLYLPEEKVLPKHSLLYETFAVYNELTKVKFIAEGLRDYQFLDSGQKKQIVNQLFKEKRKVTEKDIIHYLHNVDGYDGIELKGIEKQFNASLSTYHDLLKIIKDKEFMDDPKNEEILENIVHTLTIFEDREMIKQRLAQYDSIFDEKVIKALTRRHYTGWGKLSAKLINGICDKQTGDTILDYLIDDGKINRNFMQLINDDGLSFKEIIQKAQVVGKTDDVKQVVQELPGSPAIKKGILQSIKIVDELVKVMGYAPESIVIEMARENQTTARGKKNSQQRYKRIEDSLKNLAPGLDSNILKENPTDNIQLQNDRLFLYYLQNGKDMYTGKPLDIDQLSSYDIDHIIPQAFIKDDSIDNRVLTSSKDNRGKSDNVPSLEVVQKRKAFWQQLLDSKLISERKFNNLTKAERGGLDERDKVGFIRRQLVETRQITKHVAQILDASFNTEVNEKNQKIRTVKIITLKSNLVSNFRKEFELYKVREINDYHHAHDAYLNAVVAKAILKKYPKLEPEFVYGDYQKYDLKRYISRFKPSKEIEKATEKYFFYSNLLNFFKEEVHYADGIIVKRENIEYSKDTGEIAWNKEKDFATIKKVLSYPQVNIVKKTEIQTHGLDRGKPKGLFNSNPSPKPSEDSKENLVPIKQGLDPRKYGGYAGISNSYAVLVKAIVEKGAKKQQKTILEFQGISILDKINFENNKENYLLKKRYIEILSTITLPKYSLFEFPDGTRRRLASILSTNNKRGEIHKGNELVLPGKYTTLLYHAKNINKKLEPEHLEYVEKHRNDFAKLLECVLNFNDKYVGALKNGERIRQAFTDWETVDIEKLCFSFIGPENSKNAGLFELTSQGSASDFEFLGVKIPRYRDYAPSSLLKATLIHQSITGLYETRIDLSKLGED

SEQ ID NO: 7 - protein, 1139 aa, Streptococcus sp.
MSDLVLGLDIGIGSVGVGILNKVTGEIIHKNSRIFPAAQAENNLERRTNRQGRRLTRRKKHRRVRLNHLFEESGLITDFTKVSINLNPYQLRVKGLTAELSNEELFIALKNMVKHRGISYLDDASDDGNSSVGDYAQIVKENSKQLETKTPGQIQLERYQKYGQLRGDFTVEEDGRKHRLINVFPTSAYHAEALRILQTQQEFNPQITDEFINSYLEILTGKRKYYHGPGNEKSRTDYGKYTTKKDAQGQYITLNNIFGILIGKCTFYPEEYRAAKASYTAQEFNLLNDLNNLTVPTETKKLSEEQKYQIITYVKNEKAMGPAKLFKYIAKLLSCDVADIKGYRIDKSDKAEIHTFEAYRKMKTLETLDVKKMAREELDKLAYVLTLNTEREGIQEALDHEFADGTFSQEQVDELVQFRKANSSIFGKGWHSFSVKLMMELIPELYATSEEQMTILTRLGKQKTTSSSNKTKYIDEKQLTEEIYNPVVAKSVRQAIKIVNAAIKKYGDFDNIVIEMARETNEDDEKKAIQKIQKANKAEKDAAMRKAANQYNGKAELPHSVFHGHKQLATKIRLWHQQGERCLYTGKTISIHDLINNPNQFEIDHILPLSITFDDSLANKVLVYATANQEKGQRTPYQALDSMDDAWSFRELKAFVRESKALSNKKKEYLLTEEDISKFDVRKKFIERNLVDTRYASRVVLNALQEHFRTHKIDTKVSVVRGQFTSQLRRHWGIEKTRDTYHHHAVDALIIAASSQLNLWKKQKNTLVSYSEDQLLDIETGELISDDEYKESVFKAPYQHFVDTLKSKEFEDSILFSYQVDSKFNRKISDATIYATRKAKLDKEKKEYTYTLGKIKDIYALGTKTPSKTGFYKFLDLYKKDKSQFLMYQKDRRTWDEVIEKILEQYRPFKEKDKNGKEVDFNPFEKYRIENGPIRKYSRKGNGPEIKSLKYYDNLLGRFVDITPSESKNPVALLSLNPWRTDVYYNTETRKYEFLGLKYADLCFEKGGSYGISKVKYNKIREKEGIGKNSEFKFTLYKNDLILIKDTETNRQQIFRFWSRTGKDNPKSFEKHKLELKPYEKTRFEKGEELKVLGKVPPSSNRLQKNMQIENLSIYKVRTDVLGNQHIIKNEGDKPKLDF

SEQ ID NO: 8 - protein, 1122 aa, Streptococcus oralis
MNGLVLGLDIGIASVGVGILEKNSGKIIHANSRIFPAATADNNVERRKNRQARRLHRRKKHRGVRLQDIFEDYGLLTDFSKVSINLNPYRLRVDGLDQQLTNEELFIALKNIVKRRGISYLDDASEDGGTVSSDYGKAVEENRKLLAEKTPGQIQLERFEKYGQVRGDFTVVENGEKRRLINVFSTSAYRKEAERILRKQQEFNSKITDEFIEDCLKILTGKRKYYHGPGNEKSRTDYGRFRTDGTTLDNIFGILIGKCTFYPNEYRASKASHTAQEFNLLNDLNNLTVPTETKKLSEEQKKVIVEYAKEAKTLGASTLLKYIAKMIDASVDQISGYRVDVNNKPEMHTFEVYRKMQSLETISVGELSRNVLDELAHILTLNTEREGIEEAINTKLKDSFSQDQVLELVQFRKNNSSLFSRGWHNFSLKLMMELIPELYETSEEQMTILTRLGKQKSKETSKRTKYIDEKELTEEIYNPVVAKSVRQAIKIINEATKKYGIFDNIVIEMARENNEEDAKKDYIKRQKANQDEKNAAMEKAAFQYNGKKELPDNIFHGHKELATKIRLWHQQGEKCLYTGKNIPISDLIHNQYKYEIDHILPLSLSFDDSLSNKVLVLATANQEKGQRTPFQALDSMDDGWSYREFKSYVKESKLLGNKKKEYLLTEEDISKIEVKQKFIERNLVDTRYSSRVVLNALQDFYKAHKFDTTISVVRGQFTSQLRRKWGLEKSRETYHHHAVDALIIAASSQLRLWKKQNNPLIAYKEGQFVDSETGEILSLSDDEYKELVFKAPYDHFVDTLRSKTFEDSILFSYQVDSKYNRKISDATIYATRKAKLDKDKSEETYVLGKIKDIYSQAGYDAFIKIYNKDKSKFLMYHKDPQTFEKVIEEILRTYPSKELNDKNKEIPCNPFEKYRQENGPIRKYSKKGNGPEIKCLKYYDNKLGNYIDITPDGSDNQVVLQSIAPWRTDVYYNHKTGKYEFLGLKYSDLYFEKGTGKYKISKEKYDNIKKIEGVVETSEFKFTLYKNDLILIKDVEKGQEQLFRFLSRNNKGKHQVQLKPMNKSDFEKGEKLIDIFGTVPNSTTQCVKGLNKSNISIFKVKTDVLGKKHIIKKEGDEPKLKF

SEQ ID NO: 9 - protein, 1134 aa, Streptococcus oralis
MNGLVLGLDIGIASVGVGILEKNSGKIVHASSRIFPAATADNNVERRKNRQARRLHRRKKHRGARLKDLFEYYGLLTDFSKVSINLNPYRLRVDGLDQQLTNEELFIALKNIVKRRGISYLDDASEDGGTVSSDYGKAVEENRKLLAEQTPGQIQLERFEKYGQLRGDFTVVENSEKCRLINVFSTSAYKKEAERILRKQQEFNNQITDEFIEDYLKILTGKRKYYHGPGNEKSRTDYGRFRTDGATLDNIFGILIGKCTFYPNEYRASKASYTAQEFNLLNDLNNLTVPTETKKLSEEQKKTIIEYAKSAKTLGASTLLKYIAKMIDASVDQIRGYRVDVNNKPEMHTFEVYRKMQSLETISVGELSRNILDELAHILTLNTEREGIEEAINTKLRDSFSQDQVLELVQFRKNNSSLFSKGWHNFSLKLMMELIPELYETSEEQMTILTRLGKQKSKETSKRTKYIDEKEVTEEIYNPVVAKSVRQAIKIINEATKKHGIFDNIVIEMARENNEEDAKKDYIKRQKANQDEKYAAMEKAAFQYNGKKELPDNIFHGHKELATKIRLWHQQGEKCLYTGKSIPISDLIHNQYKYEIDHILPLSLSFDDSLSNKVLVLATANQEKGQRTPFQALDSMDDAWSYREFKSYVKESKLLGNKKKEYLLTEEDISKIEVKQKFIERNLVDTRYSSRVVLNALQDFYKNHNFDTTISVVRGQFTSQLRRKWGLEKSRETYHHHAVDALIIAASSQLRLWKKQNNPLIAYKEGQFVDSQTGEIISLTDDEYKELVFKAPYDHFVDTLSSKTFEDSILFSYQVDSKFNRKISDATIYATRKAKLDKEKKEYTYTLGKIKDIYSLGTKTPSKTGFYKFLDLYNKDKSQFLMFQKDRKTWDEVIEKIMEQYRPFKEYDKAGKLVDFNPFEKYRQENGPIRKYSKKGNGPEIKSLKYYDILLGKHKNITPEGSRNTVALLSLNPWRTDVYYNMETKKYEFLGLKYADLPFEEGGAYGISTETYNELREKEGIGKNSEFKFTLYKNDLILIKDTETNCQQFFRFWSRTGKDNPKSFEKHKIELKPYEKAKFEKGEELEVLGKVPPSSNQFQKNMQIENLSIYKVKTDVLGNKHFIKKEGDKPKLKF

SEQ ID NO: 10 - protein, 1054 aa, Staphylococcus warneri
MKEKYILGLDLGITSVGYGIINFETKKIIDAGVRLFPEANVDNNEGRRSKRGSRRLKRRRIHRLDRVKSLLTEYNLINREQIPTSNNPYQIRVKGLSEILSKDELAIALLHLAKRRGIHNINVSSEDEDASNELSTKEQINRNNKLLKNKYVCEVQLQRLKEGQIRGEKNRFKTTDILKEIDQLLKVQKDYHNLDIDFINQYKEIVETRREYFEGPGQGSPFGWNGDLKKWYEMLMGHCTYFPQELRSVKYAYSADLFNALNDLNNLIIQRNDSEKLEYHEKYHIIENVFKQKKKPTLKQIAKEIGVNPEDIKGYRITKSGTPQFTEFKLYHDLKSIVFDKSILENEAILDQIAEILTIYQDEESIKEELNKLPEILNEQDKAEIAKLTGYNVTHRLSLKCIHLINEELWQTSRNQMEIFNYLNIKPNKVDLSEQNKIPKDLVDEFILSPVVKRTFIQSINVINKVIEKYGIPEDIIIELARENNSDDRKKFINNLQKKNEATRKRINEIIGQTGNQNGKRIVEKIRLHDQQEGKCLYSLESIPLMDLLNNPQNYEVDHIIPRSVAFDNSIHNKVLVKQIENSKKGNRTPYQYLNSSDANLSYNQFKQHILNLSKSKDRISKKKKDYLLEERDINKFEVQKEFINRNLVDTRYATRELTSYLKAYFSANNMDVKVKTINGSFTNHLRKVWRFDKYRNHSYKHHAEDALIIANADFLFKENKKLQNANKILEKPTIENDTQKVTVEKEEDYNNMFETPKLVEDIKQYRDYKFSHRVDKKPNRQLIKDTLYSTRMKDEHNYIVQTITDIYGKDNTNLKKQFNKNPEKFLMYQNDPKTFEKLSIIMKQYSDEKNPLAKYYEETGEYLTKYSKKNNGPIVKKIKLLGNKVGNHLDVTNKYENSTKKLVKLSIKNYRFDVYLTEKGYKFVTIAYLNVFKKDNYYYIPKDLYQELKAKKKIKDTDQFIASFYKNDLIKLNGDLYKIIGVNSDDRNIIELDYYDIKYKDYCEINNIKGEPRIKKTIGKKTESIEKLTTDVLGNLYLHTTEKAPQLIFKRGL

SEQ ID NO: 11 - protein, 1053 aa, Staphylococcus sciuri
MKRNYILGLDIGITSVGYGIIDYETRDVIDAGVRLFKEANVENNEGRRSKRGARRLKRRRRHRLQRVKKLLFDYNLLTDHSELSGINPYEARVKGLSQKLSEVEFSAALLHLAKRRGVHNVNEVEEDTGNELSTKEQISRNSKALEEKYVAELQLERLKTDGEVRGPNNRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVIARDENEKLEYYEKFQIIENVFKQKKKPTLKQIAKELLVNEEDIKGYRVTSIGKPEFTNFKIYHDIKGITERKEVLENAELLDQIAEILTIYQSSEDVQEELANLNSELTQEEIEQISNLKGYTGTHNLSLKAINLILDELWHTNDNQMAIFNRLKLVPKKVDLSQQKEIPTTLVDDFILSPVVKRSFIQSIKVINAIIKKYGLPNDIIIELAREKNSKDAQKMINEMQKRNRQMNERIEEIIRTTGKENAKYLIEKIKLHDMQEGKCLYSLESIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRELMNLLRSYFRVNNLDVKVKSINGGFTSFLRRKWKFKKERNKGYKHHAEDALIIANADFIFKEWKKLDKAKKVMENQTVEEKQAESMPEIETEQEYKEIFITPHQIQHIKGFKDYKYSHRVDKKPNRELINDTLYSTRKDDKGNTLIVNNLNGLYDKDNDKLKKLMNKSPEKLLMYHHDPQTYQKLKLIMEQYGDEKNPLYKYYEETGNYLTKYSKKDNGPVIKKIKYYGKNLKAHLDITDDYPNSRNKVVKLSVKPYRFDVYLDNDIYKFVTVKNLDVIKKEDYYEVNSKCYKEAKKLKKISDQAEFIASFYNNDLIKINGELYRVIGVNNDLLNRIEVNMINITYREYLENMNDKRPPRIIKTIASKTQSIKKYSTDILGNLYEVKSKQKPQMIMKG

SEQ ID NO: 12 - protein, 1130 aa, Streptococcus gallolyticus
MTNGKILGLDIGIASVGVGIIEAKTGKVVHANSRLFSAANAENNTERRGFRGSRRLNRRKKHRVKRVRDLFEKHEIVTDFRNLNLSPYELRVKGLTEQLTNEELFAALRTISKRRGISYLDDAEDDSTGSTDYAKSIDENRRLLKNKTPGQIQLERLEKYGQLRGNFTVYDENGEAHRLINVFSTSDYEKEARKILETQADYNKKITAEFIDDYVEILTQKRKYYHGPGNEKSRTDYGRFRTDGTTLENIFGILIGKCNFYPDEYRASKASYTAQEYNFLNDLNNLKVPTETGKLPTEQKESLVEFAKNTATLGPSKLLKEIAKILDCKVDEIKGYREDDKGKPDLHTFEPYRKLKFNLESINIDDLSREVIDKLADILTLNTEREGIEDAIKRNLPNQFTEEQISEIIKVRKSQSTAFNKGWHSFSAKLMNELIPELYATSDEQMTILTRLEKFKVNKKSSKNTKTIDEKEVTDEIYNPVVAKSVRQTIKIINAAVKKYGDFDKIVIEMPRDKNADDEKKFIDKRNKENKKEKDDALKRAAYLYNSSDKLPDEVFHGNKQLETKIRLWYQQGERCLYSGKPIPIHDLVHNSNNFEIDHILPLSLSFDDSLANKVLVYDWANQEKGQKTPYQVIDSMDAAWSFREMKDYVLKQKGLGKKKRDYLLTTENIDKIEVKKKFIERNLIDTRYASRVVLNSLQSALRELCKDTKVSVIRGQFTSQLRRKWKIDKSRETYHHHAVDALIIAASSQLKLWEKQDNLMFIDYGNNQVVDKETGEILSVSDDEYKELVFQPPYQGFVNTISSKGFEDEILFSYQVDSKYNRKVSDATIYSTRKAKIGKDKKEETYVLGKIKDIYSQNGFDTFIKKYNKDKTQFLMYQKDPLTWENVIEVILRDYPTTKKSEDGKNDVKCNPFEEYRRENGLICKYSKKGKGTPIKSLKYYDKKLGNCIDITPEESRNKVILQSINPWRADVYFNPETLKYELMGLKYSDLSFEKGTGKYHISQEKYDAIKEKEGIGKKSEFKFTLYRNDLILIKDTASGEQEIYRFLSRTMPNVNHYAELKPYDKEKFDGGQELMEVFGKVANGGQCLKSLNKSNISIYKVRTDVLGNKYFVKKEGDKPKLNFKNNKK

SEQ ID NO: 13 - protein, 1396 aa, unidentified
MKRVNEDYILGLDIGTNSCGWAVTDKKNNLLKLRGKTAIGSHLFEEGHTAADRRGFRTTRRRLKRRKWRLRLLEEIFAEPMAKVDPGFFVRLHQSWVSPLDKDRKKYNAIVFPTAKEDQAFYKHYATIYHLRDELMTQDRQFDLREIFLAIHHIVKYRGNFLQDTPVKDFEASKIEVGPILSHINNAFAEKIVEDQDPIELNVANAADIEDVIRGKDAEKTVYKLDKVKKIAKLLTDSTAKEEKNVAKQIANAIMGYKTQFETILDKEIDKSDKAQWEFKLSDADADDKLDALLPDLDETDQTVVAEIEKLFSAITLSTIVDENKSLSQSMVEKYKKHKKDYKKLKKYINTLQDQTKAKKLLLAYDLYVNNRHGRLLEAKKTFEDKKKKKALTKDEFYKIVKDNLDDSDLAHEIQQEIAADNFMPKQRTNSNGVIPFQLHQIELDKIIANQGKYYPFLAAENPVEDHRKQAPYKLDELVRFRVPYYVGPMITADEQEKTSGKSFAWMVRKEDGQITPWNFEQKVDRQESANKFIKRMTIKDTYLLSEDVLPANSLLYQRFEVLNELNNIRINGSRISVDLKQQIFNDLFEEKKTVTEKSLTSYLKQNLHLPTVEIKGLADPTKFNSSLASYYHLKSLHVFDKELADPQYQKDFEKIIEYSSIFEDKKIFQDKLHAEFKWLTPEQFKAISTWRLQGWGRLSRKLLVELHDTNGQNIMEQLWDSQKNFMQIVTEPDFKDAIAKENQNVTRANGVEEILADAYTSPANKKAIRQVVKVVADVVKAAGGKKPAQFAIEFTRDPDKNPQLSHIRGTKLLKAYQETAGELVDQKLTDSLKEAMTSRKLLKDKYFLYFMQAGRDAYTGQKINIDEVSTNYQIDHILPQSFIKDDSFDNRVLTATPLNAEKSDDVPYKRFANNYVSDMKMTVGEMWKHWQKAGIINKHKLGNLLLDPDRLNKFQKSGFINRQLVETSQIIKLVSVILQNKYPDAEIITVKAGDNSALRQRLNLYKSRDVNDYHHAIDAYLSIICGNFLYQVYPKYRPYFVYGKYKKFSQDPDLQKEVIKHFKGFTFMWPLLQKDNSERKAPEKIKENNSDRIVFYKHPDIFDKLRKAYNYKYMLVSRETTTENSGLFDVTIYPRGERDLAKTRKLIPKSNGLDPKIYGGYSGNTDAYMVIVKIDKGKESIYKVIGVPMRALASLNRAKKQGNYKEELHQVLEPQIMFDKNGKPKRSVKGFRIIKDHVPFKQVVLDGDKKFMLNSSTYEINAKQLTLTPETMRIVTDNLKKGEDQDQLLVKAYDEILQKVDQYLPLFDVNKFRNSLHLGRAKFLDLAVNDKKITLTNILNGLHDNLVTPDLKNIGIKTPLGKLQVPSGIVLSSEAILIFQSPTGLFEKRVRIADL

SEQ ID NO: 14 - protein, 1122 aa, Streptococcus suis
MSNGKILGLDIGIASVGVGVIDAQTGEIIHASSRIFPSANAANNAERRTFRGSRRLIRRKKHRIKRLDDLFNDFHINLDGEMSTDNPYVLRVKGLSQKLTVEELYISIKNIMKRRGISYLDDAESDNEAGRSDYAKAIERNRQLLTSKTPGEIQLERLEKYGQLRGNFTIIDEEGQSQQIINVFSTSDYVKEVEKILDCQKMYHKFISDEFCDKLIELLREKRKYYVGPGNEKSRTDYGIYRTDGTTLENLFGILIGKCTFYPDQYRSSRASYTAQEFNFLNDLNNLTVPTETKKLSQEQKEFLVNYAKETSVLGAGKILQQIAKLADCKVEDIRGYRLDNKDKPELHTFETYRAMKGLVPLVDIGVLSREQLDILADILTLNTDFEGIREALKKQLPNVFDEKQVKGLASFRKSKSQLFAKGWHNLSQKIMLEVIPELYATSDEQMTILTRLGKFEKSSVAEYPSSINVDEITDEIYNPVVAKSIRQTIKIINASIKKWDEFDQIVIEMPRDRNEDEEKKRIADGQKANAKEKADSILRAAELYCAGKVLPDYVYNGHNQLATKIRLWYQQGERCIYTGQPISIHDLIHNQNQYEIDHILPLSLTFDDSLSNKVLVLATANQEKAQRTPYNYLKSATSAWSYREFKDYVTKRKGIGKKKCEYLTFEEDINGFEVRSKFIQRNLVDTRYASKVILNALQDYFKISGIQTKVSVVRGQFTSQLRHKWGIEKTRETYHHHAVDALIIAASSQLRLWKKQESPLVVDYQEGRQVDLETGEILELTDEQYKELVYQPPYQGFVNTISSSAFDNEILFSYQVDSKVNRKISDATIYATRNAQLGKDKTEGIYVLGKIKDIYTQAGYEAFLKRYTKDKTSFLMYHKDLDTWEKVIEIILRDYREYDEKGKEIGNPFERYRRENGYVKKYSRKGNGTAIKSLKYYDNKLGNHIDITPENSRNAVVLQSLKPWRTDVYFNKETGKYEFLGIKYSDLSFEKGTGEYGISQEKYDSIKIAEGVAKKSIFKFTLYKQDLLFIKDIENNFGKLLRFTSKNDTSKHYVELKPYDKNKFGTEEPLLPVLGNVAKSGQCIKGLNKSNISIYKVRTDILGYRHFIKQEGEHPQLKFKK

SEQ ID NO: 15 - protein, 1453 aa, synthetic construct
MPKKKRKVGTMEKSYSIGLAIGTNSVGWSVITDDYKVPAKKMRVLGNTDKKYIKKNLLGALLFDSGETAEATRLKRTARRRYTRRKNRLRYLQEIFAKEMAKVDESFFQRLDESFLTDDDKTFDSHPIFGNKAEEDAYHQKFPTIYHLRKHLADSQEKADLRLIYLALAHMIKFRGHFLYDNFNDDNFDWRNIDIQKRYEEFIETYDSTLGESYLADISVDAASILEEKVSKTERLENLLKYYPTEKKTTFFGNLIKLILGQQAKFKAIFNLEDEISLQFATPTYDEDLEELLGKIDNGDSYSELFVAAQNLYNTILLASFLKTDNKSAKAPLSTSMIERYENHKKDLAKLKDFVKKNCPDQYHDIFRDKSKNGYAGYIDNGVSQNDFYTFIGKCLEESLKKDKGAQYFLDKIDRDDFLRKQRTFDNGAFPYQIHLQEMHAILRRQGDYYPFLKENQDKIEKILTFRIPYYVGPLARKDSRFAWANYRSDEAITPWNFDEVVDKEKSAEKFITRMTLNDLYLPEEKVLPKHSLIYETFTVYNELTNIKYVNDQGNAIHFDSELKEKIFNQLFKENRKVSKKTLIDFLNNSEGIYTDKLVGIDEEVKYLNASLGTYHDLKKILESFMDDEINEKIIEDIIQTLTLFEDIEMKRQRLQKYDDIFTPKQLKELARRNYTGWGRLSYKLINGIRNKENNKTILDYLKNGNRNFMQLINDDRLSFKQIIIDARKIEKLDNIESVVYNLPGSPAIKKGILQSIKIVDELVKVMGHNPDNIVIEMARENQTTNQGKNRSQQRLKRLQDSMSNFKDSSISLKDVDNSDLQNDRLFLYYIQNGKDMYTGEELDIDHLSDYDIDHIIPQSFIKDNSIDNRVLTSSAKNRGKSDDVPGRDVVLKMKPFWKKLYDVKLISKRKFDNLTKSEHGGLTESDKAGFIKRQLVETRQITKYVAQILDGRFNTKRDDNNKVIRDVKVITLKSSLVSQFRKDFGFYKVREINDYHHAHDAYLNAVVGTAILKKYPKLAPEFVYGEYKKCDVRKLIAKSGDKSEIGKATAKYFFYSNLMNFFKRVIRYSNGMIVVRPVIEYSKDTGEIAWDKEKDFKTVCKVLSCPQVNIVKKVEKQSHGLDRGKPKGFYNANPSPKPKKGSKVNLVPIKANLNPKNYGGYAGISNSYAVLVDATIEKGAKKKLTRIQEFQGISIIDREKYEKNKVEFLKGLGYKEIYSIITLPKYSLFELADGSRRMLASILSTNNKRGEIHKGNELVLPAKYIPLLYHANRIHNTFETGHREYVEKHIAEFKEIAEIILEFNNKYVNAKKNSSIIEKALESFDSFSLDEICDSFVGKLKKNNTKKNSGLFELVSLGSASDFEFLETKVPRYRDYTPSSLLNATLIHQSITGLYETRIDLSKLGEEKRPAATKKAGQAKKKKGSYPYDVPDYAYPYDVPDYAYPYDVPDYA

SEQ ID NO: 16 - protein, 1391 aa, synthetic construct
MPKKKRKVGTMTKDYTIGLAIGTNSVGWAVLTDDYQLMKRKMSVHGNTEKKKIKKNFWGARLFDEGQTAEFRRTKRTNRRRLARRKYRLSKLQDLFAEELCKQDDCFFVRLEESFLVPEEKQYKPASIFPTLEEEKEYYQKYPTIYHLRQKLVDSTEKEDLRLVYLALAHLLKYRGHFLFEGDLDTENTSIEESFRVFLEQYSKQSDQPLIVHQPVLTILTDKLSKTKKVEEILKYYPTEKINSFFAQCLKLIVGNQANFKRIFDLEAEVKLQFSKETYEEDLESLLEKIGDEYLDIFLQAKKVHDAILLSEIISSTVKHTKAKLSSGMVERYERHKADLAKFKQFVKENVPQKSTAFFKDTTKNGYAGYIKGKTTQEEFYKFVKKELSGVVGSEPFLEKIDQETFLLKQRTYTNGVIPHQVHLIELKAIIDQQKQHYPFLEEAGPKIIALFKFRIPYYVGPLAKEQEASSFAWIERKTAEKIHPWNFSEVVDIEKSAMRFIQRMTKQDTYLPTEKVLPKNSLLYQKYMIFNELTKVSYKDERGVKQYFSGDEKQQIFKQLFQKERGKITVKKLQNFLYTHYHIENAQIFGIEKAFNASYSTYHDFMKLAKTNQKAMQEWLEQPEMEPIFEDIVKILTIFEDRQMIKHQLSKYQEVFGEKLLKEFARKHYTGWGRFSAKLIHGIRDRKTNKTILDYLINDDDVPANRNRNLMQLINDEHLSFKEEIAKATVFSKHKSLVDVIQDLPGSPAIKKGIWQSLKIVEELIAIIGYKPKNIVIEMARENQKTHRTKPRLKALENGLKQIGSTLLKEQPTDNKALQKERLYLYYLQNGRDMYTGEPLEIENLHQYEVDHIIPRSFIVDNSIDNKVLVARKQNQKKRDDVPKKQIVNEQRIFWNQLKEAKLISPKKYAYLTKIELTPEDKARFIQRQLVETRQITKHVANILHQSFHQEEEGTDCDGVQIITLKATLTSQFRQTFGLYKVREINPHHHAHDAYLNGFIANVLLKRYPKLAPEFVYGKYVKYSLARENKATAKKEFYSNILKFFESDEPFCDENGEIYWEKSHHLPRIKKVLSSHQVNVVKKVEQQKGGFYKETVNSKEKPDKLIERKNNWDVTKYGGFGSPVIAYAIAFVYAKGKTQKKTRAIEGITIMEQAAFEKDPTTFLKEKGFPQVTEFIKLPKYTLFEFDNGRRRFLASHKESQKGNPFILSDQLVTLLYHAQHYDKITYQESFDYVNTHLSDFSAILTEVLAFAEKYTLADKNIERIQELYEENKYGEISMIAQSFLQLLQFNAIGAPADFKFFGVTIPRKRYTSLTEIWDATIIYQSVTGLYETRIRMGDLWAGEQKRPAATKKAGQAKKKKGSYPYDVPDYAYPYDVPDYAYPYDVPDYA

SEQ ID NO: 17 - protein, 1185 aa, synthetic construct
MPKKKRKVGTMTNGKILGLAIGVASVGVGIIEAKTGKVIHANSRLFSAANAENNAERRGFRGARRLTRRKKHRVKRVRDLFEKYDISTDFRNLNLNPYELRVKGLSEQLTNEELFAALRTIAKRRGISYLDDAEDDSTGSSDYAKSIDENRRLLKSMTPGQIQLERLEKYGQLRGNFTVYDENGEAHRLINVFSTSDYKNEARKILETQSNYNKQITDEFIEDYIEILTQKRKYYHGPGNEKSRTDYGRFRTDGTTLENIFGILIGKCSFYPEEYRASKASYTAQEFNFLNDLNNLKVPTETGKLSTEQKEYLVDFAKNSKTLGASKLLKEIAKLVDGDVKDISGYREDSKGKPDLHTFEPYRKLKFNLTTVDIDNLSRDILDKLANILTLNTEREGIEDAINRNLPEQFTKEQISEIVQIRKSQSSAFNKGWHSFSAKLMNELIPELYATSEEQMTILTRLEKFKATKKSSKNTKTIDEKEITDEIYNPVVAKSVRQTIKIINAAVKKYGDFDKIVIEMPRDKNADDEKKFIDKKNKENKKEKDDSLKRAAYLYNGTDKLPDDVFHGNKQLATKIRLWYQQGERCLYSGKPILIQDLVHNSNNFEIDHILPLSLSFDDSLANKVLVYAWTNQEKGQKTPYQVIDSMDAAWSFREMKDYVLKQKGIGKKKREYLLTTENIDKIEVKKKFIERNLVDTRYASRVVLNSLQTALKELGKDTKVSVVRGQFTSQLRRKWKIDKSRETYHHHAVDALIIAASSQLKLWQKHENLMFENYGENQVVNKETGEILSISDDEYKELVFQPPYQGFVNTISSKAFEDEILFSYQVDSKFNRKVSDATIYSTRKAKLGKDKKEETYVLGKIKDIYSQDGFDTFIKRYKKDKTQFLMYQKDPLTWENVIEVILRDYPTTKKSEDGKNDVKCNPFEEYRRENGLICKYSKKGKGTPIKSLKYYDKKLGNHISITPKESKNDVVLQSLNPWRADLYFNPDTLKYELMGLKYSDLSFEKGTGKYHISQEKYDEIKEKEGIGQNSEFKFTLYRNDLILIKDTESGEQEIYRFLSRTMPNVKHYVELKPYDKEKFNGGQELIKSLGEADKVGRCLKGLSKPGISIYKVRTDVLGNKFFVKKEGDKPKLDFKNNKKKRPAATKKAGQAKKKKGSYPYDVPDYAYPYDVPDYAYPYDVPDYA

SEQ ID NO: 18 - protein, 1108 aa, synthetic construct
MPKKKRKVGTMNNSYILGLAIGITSVGYGIIEYETRDVIDAGVRLFKEANVENNEGRRSKRGARRLKRRRRHRLQRVKKMLFDYKLLNEDSEISGINPYEARVKGLSEKLSDEEFSAALLHLAKRRGVHNVSDVEEDTGNELSTKEQIARNNKALEDKYVAELQLERLKEQGEVRGAANRFKTSDYIKEAKQLLKTQSDYHKIDETFIETYISLLETRRTYYEGPGEGSPFGWKDIKEWYEMLMGHCTYFPEELRSVKYAYNADLYNALNDLNNLVIARDENEKLEYYEKFQIIENVFKQKKKPTLKQIAKEILVNEEDIKGYRVTSTGKPEFTNFKIYHDIKGITSRKEILENADLLNQIAEILTIYQSAEDIQEELAELEPKLTQEEIEQISNLTGYTGTHRLSLKAINLILDELWNTSDNQMTIFNRLKLVPKKVDLSQQKEIPTSLVDDFILSPVVKRSFIQSIKVINAIIKKFGLPKDIIIELAREKNSKEAQKFINEMQKRNRQTNERIEKIIKETGKEKAKFLIEKIKLHDMQEGKCLYSLESIPLEDLLNNPYHYEVDHIIPRSVSFDNSFNNKVLVKQEENSKKGNRTPFQYLSSSDAKISYETFKKHILNLSKGKGRVSKKKKEYLLEERDINRFSVQKDFINRNLVDTRYATRELMNLLRSYFRVNDLDVKVKSINGGFTSFLRTKWKFKKERNQGYKHHAEDALVIANADFIFKEWKKLDTTNKVMENQTVEEQQAENMPGIETDDEYKEIFVIPRQIQSIKDFKDYKYSHRVDKKPNRELVNDTLYSTRKDDKGNTLIINNIKGLYDKDNDKLKNLIKKSPEKLLMYHHDPQTYQKLKTIMEQYSNEKNPLYKYHEETGNYLTKYSKKDNGPIIKKVKYYGKKLNAHLDITNDYSNSQNKIVKLSLKPYRFDVYLDNGGYKFVTVKNLDVIKKEGFFKIDSNAYEKAKSEKKIDENAVFIASFYNNDLIKIDGELYRIVGVNNDTRNVVELNMIPITYKEYLENINDKRTPRILKTISQKTYSIEKYSTDILGNLYKVKSKKKPQMIMKGKRPAATKKAGQAKKKKGSYPYDVPDYAYPYDVPDYAYPYDVPDYA

SEQ ID NO: 19 - protein, 1180 aa, synthetic construct
MPKKKRKVGTMNGLVLGLAIGIASVGVGILNKETGEIIHVNSRIFPAATADSNVERRGFRQGRRLGRRKKHRSARLNDLFEEFGFITDFSAVPLNLNPYALRVKGLSEELTNEELFIALKNIIKRRGISYLDDASEDGETASNEYGKAVEENRKLLADKTPGQIQLERFEKYGQVRGDFTVVENGENHRLINVFSTSAYKKEAERILRRQQEFNVRISDEFIEAYLTILTGKRKYYHGPGNEKSRTDYGRFRTDGTTLDNIFGILIGKCTFYPDEYRAAKASYTAQEFNLLNDLNNLTVPTETKKLRPEQKRQIVEYARTAKTLGTPTLLKYIAKLVDGSIDDIKGYRIDKSDKPEMHTFDAYRKMRTLELVDVDILSRETLDDLAYILTLNTESEGILEALNSKMPGTFTKEQIDELIQFRKKNSAVFGKGWHNFSLKLMNELISELYETSEEQMTILTRLGKQRSREISKRTKYIDEKELTDEIYNPVVAKSVRQAIKIINLATKKYGIFDNIVIEMARESNEDDEKKAIQNVQKANEDEKKAAMEKAADLYNGKKELPDSIFHGHKELATKIRLWHQQGERCLYTGKNISIHDLIHNPHQYEIDHILPLSLSFDDGLANKVLVLATANQEKGQRTPFQALDSMDDAWSYIEFKQYVRNSKSLSNKKKDYLLTEEDISKIEVKQKFIERNLVDTRYSSRVVLNTLQEFYKTNDFDTKISVVRGQFTSQLRRKWKIEKSRDTYHHHAVDALIIAASSQLRLWKKQNNPLISYREGQLVDPETGEILSLTDDEYKELVFRPPYDYFVDTLKSKSFEDSILFSYQVDSKYNRKISDATIYGTRKAQLGKDKQEETYVLGKIKDIYSQKGYEDFIKRYKKDTTQFLMYHKDPQTFAKVIEEILKTYPDKELNEKGKEIPCNPFEKYRQENGPIRKYSKKGKGPEIKSLKYYDNKLGNHIDITPVNSQNQVVLQSLKPWRTDVYFNPQTSKYELMGLKYSDLRFEKGSGSYGISPEKYNKVKAKEGVDEDSEFKFTLYKNDLILIKDTETGEQQLFRYGSRNDTSKHYVELKPYEKAKFEGNQQLMNLLGTVAKGGQCLKGINKPNLSIYKVKTDVLGNKHFIKKEGDQPQLNFKKKIKRPAATKKAGQAKKKKGSYPYDVPDYAYPYDVPDYAYPYDVPDYA

SEQ ID NO: 20 - protein, 1447 aa, synthetic construct
MPKKKRKVGTMENKNYSIGLAIGTNSVGWAVITDDYKVPSKKMKVFGNTDKHFIKKNLIGALLFDEGATAEDRRLKRTARRRYTRRKNRLRYLQEIFSEEISKLDSSFFHRLDDSFLVPKDKRGSKYPIFATLEEEKEYHKKFPTIYHLRKHLADSKEKTDLRLIYLALAHMIKYRGHFLYEESFDIKNNDIQKIFNEFISIYDNTFEGSSLSGQNAQVEAIFTDKISKSAKRERVLKLFSDEKSTSLFSEFLKLIVGNQADFKKHFDLEEKAPLQFSKDTYDEDLENLLGQIGDGFTDLFLVAKKLYDAILLSGILTVTDPSTKAPLSASMIERYESHQKDLAALKQFIKNNLPKRYNEVFSDQSKDGYAGYIDGKTTQEAFYKYIKNLLSKFEGADYFLDKIEREDFLRKQRTFDNGSIPHQIHLQEMNAILRRQGEHYPFLKENREKIEKILTFRIPYYVGPLARGNRDFAWLTRNSDQAIRPWNFEEIVDKASSAEEFINKMTNYDLYLPEEKVLPKHSLLYETFAVYNELTKVKFIAEGLRDYQFLDSGQKKQIVNQLFKEKRKVTEKDIIHYLHNVDGYDGIELKGIEKQFNASLSTYHDLLKIIKDKEFMDDPKNEEILENIVHTLTIFEDREMIKQRLAQYDSIFDEKVIKALTRRHYTGWGKLSAKLINGICDKQTGDTILDYLIDDGKINRNFMQLINDDGLSFKEIIQKAQVVGKTDDVKQVVQELPGSPAIKKGILQSIKIVDELVKVMGYAPESIVIEMARENQTTARGKKNSQQRYKRIEDSLKNLAPGLDSNILKENPTDNIQLQNDRLFLYYLQNGKDMYTGKPLDIDQLSSYDIDHIIPQAFIKDDSIDNRVLTSSKDNRGKSDNVPSLEVVQKRKAFWQQLLDSKLISERKFNNLTKAERGGLDERDKVGFIRRQLVETRQITKHVAQILDASFNTEVNEKNQKIRTVKIITLKSNLVSNFRKEFELYKVREINDYHHAHDAYLNAVVAKAILKKYPKLEPEFVYGDYQKYDLKRYISRFKPSKEIEKATEKYFFYSNLLNFFKEEVHYADGIIVKRENIEYSKDTGEIAWNKEKDFATIKKVLSYPQVNIVKKTEIQTHGLDRGKPKGLFNSNPSPKPSEDSKENLVPIKQGLDPRKYGGYAGISNSYAVLVKAIVEKGAKKQQKTILEFQGISILDKINFENNKENYLLKKRYIEILSTITLPKYSLFEFPDGTRRRLASILSTNNKRGEIHKGNELVLPGKYTTLLYHAKNINKKLEPEHLEYVEKHRNDFAKLLECVLNFNDKYVGALKNGERIRQAFTDWETVDIEKLCFSFIGPENSKNAGLFELTSQGSASDFEFLGVKIPRYRDYAPSSLLKATLIHQSITGLYETRIDLSKLGEDKRPAATKKAGQAKKKKGSYPYDVPDYAYPYDVPDYAYPYDVPDYA

SEQ ID NO: 21 - protein, 1194 aa, synthetic construct
MPKKKRKVGTMSDLVLGLAIGIGSVGVGILNKVTGEIIHKNSRIFPAAQAENNLERRTNRQGRRLTRRKKHRRVRLNHLFEESGLITDFTKVSINLNPYQLRVKGLTAELSNEELFIALKNMVKHRGISYLDDASDDGNSSVGDYAQIVKENSKQLETKTPGQIQLERYQKYGQLRGDFTVEEDGRKHRLINVFPTSAYHAEALRILQTQQEFNPQITDEFINSYLEILTGKRKYYHGPGNEKSRTDYGKYTTKKDAQGQYITLNNIFGILIGKCTFYPEEYRAAKASYTAQEFNLLNDLNNLTVPTETKKLSEEQKYQIITYVKNEKAMGPAKLFKYIAKLLSCDVADIKGYRIDKSDKAEIHTFEAYRKMKTLETLDVKKMAREELDKLAYVLTLNTEREGIQEALDHEFADGTFSQEQVDELVQFRKANSSIFGKGWHSFSVKLMMELIPELYATSEEQMTILTRLGKQKTTSSSNKTKYIDEKQLTEEIYNPVVAKSVRQAIKIVNAAIKKYGDFDNIVIEMARETNEDDEKKAIQKIQKANKAEKDAAMRKAANQYNGKAELPHSVFHGHKQLATKIRLWHQQGERCLYTGKTISIHDLINNPNQFEIDHILPLSITFDDSLANKVLVYATANQEKGQRTPYQALDSMDDAWSFRELKAFVRESKALSNKKKEYLLTEEDISKFDVRKKFIERNLVDTRYASRVVLNALQEHFRTHKIDTKVSVVRGQFTSQLRRHWGIEKTRDTYHHHAVDALIIAASSQLNLWKKQKNTLVSYSEDQLLDIETGELISDDEYKESVFKAPYQHFVDTLKSKEFEDSILFSYQVDSKFNRKISDATIYATRKAKLDKEKKEYTYTLGKIKDIYALGTKTPSKTGFYKFLDLYKKDKSQFLMYQKDRRTWDEVIEKILEQYRPFKEKDKNGKEVDFNPFEKYRIENGPIRKYSRKGNGPEIKSLKYYDNLLGRFVDITPSESKNPVALLSLNPWRTDVYYNTETRKYEFLGLKYADLCFEKGGSYGISKVKYNKIREKEGIGKNSEFKFTLYKNDLILIKDTETNRQQIFRFWSRTGKDNPKSFEKHKLELKPYEKTRFEKGEELKVLGKVPPSSNRLQKNMQIENLSIYKVRTDVLGNQHIIKNEGDKPKLDFKRPAATKKAGQAKKKKGSYPYDVPDYAYPYDVPDYAYPYDVPDYA

SEQ ID NO: 22 - protein, 1177 aa, synthetic construct
MPKKKRKVGTMNGLVLGLAIGIASVGVGILEKNSGKIIHANSRIFPAATADNNVERRKNRQARRLHRRKKHRGVRLQDIFEDYGLLTDFSKVSINLNPYRLRVDGLDQQLTNEELFIALKNIVKRRGISYLDDASEDGGTVSSDYGKAVEENRKLLAEKTPGQIQLERFEKYGQVRGDFTVVENGEKRRLINVFSTSAYRKEAERILRKQQEFNSKITDEFIEDCLKILTGKRKYYHGPGNEKSRTDYGRFRTDGTTLDNIFGILIGKCTFYPNEYRASKASHTAQEFNLLNDLNNLTVPTETKKLSEEQKKVIVEYAKEAKTLGASTLLKYIAKMIDASVDQISGYRVDVNNKPEMHTFEVYRKMQSLETISVGELSRNVLDELAHILTLNTEREGIEEAINTKLKDSFSQDQVLELVQFRKNNSSLFSRGWHNFSLKLMMELIPELYETSEEQMTILTRLGKQKSKETSKRTKYIDEKELTEEIYNPVVAKSVRQAIKIINEATKKYGIFDNIVIEMARENNEEDAKKDYIKRQKANQDEKNAAMEKAAFQYNGKKELPDNIFHGHKELATKIRLWHQQGEKCLYTGKNIPISDLIHNQYKYEIDHILPLSLSFDDSLSNKVLVLATANQEKGQRTPFQALDSMDDGWSYREFKSYVKESKLLGNKKKEYLLTEEDISKIEVKQKFIERNLVDTRYSSRVVLNALQDFYKAHKFDTTISVVRGQFTSQLRRKWGLEKSRETYHHHAVDALIIAASSQLRLWKKQNNPLIAYKEGQFVDSETGEILSLSDDEYKELVFKAPYDHFVDTLRSKTFEDSILFSYQVDSKYNRKISDATIYATRKAKLDKDKSEETYVLGKIKDIYSQAGYDAFIKIYNKDKSKFLMYHKDPQTFEKVIEEILRTYPSKELNDKNKEIPCNPFEKYRQENGPIRKYSKKGNGPEIKCLKYYDNKLGNYIDITPDGSDNQVVLQSIAPWRTDVYYNHKTGKYEFLGLKYSDLYFEKGTGKYKISKEKYDNIKKIEGVVETSEFKFTLYKNDLILIKDVEKGQEQLFRFLSRNNKGKHQVQLKPMNKSDFEKGEKLIDIFGTVPNSTTQCVKGLNKSNISIFKVKTDVLGKKHIIKKEGDEPKLKFKRPAATKKAGQAKKKKGSYPYDVPDYAYPYDVPDYAYPYDVPDYA

SEQ ID NO: 23 - protein, 1189 aa, synthetic construct
MPKKKRKVGTMNGLVLGLAIGIASVGVGILEKNSGKIVHASSRIFPAATADNNVERRKNRQARRLHRRKKHRGARLKDLFEYYGLLTDFSKVSINLNPYRLRVDGLDQQLTNEELFIALKNIVKRRGISYLDDASEDGGTVSSDYGKAVEENRKLLAEQTPGQIQLERFEKYGQLRGDFTVVENSEKCRLINVFSTSAYKKEAERILRKQQEFNNQITDEFIEDYLKILTGKRKYYHGPGNEKSRTDYGRFRTDGATLDNIFGILIGKCTFYPNEYRASKASYTAQEFNLLNDLNNLTVPTETKKLSEEQKKTIIEYAKSAKTLGASTLLKYIAKMIDASVDQIRGYRVDVNNKPEMHTFEVYRKMQSLETISVGELSRNILDELAHILTLNTEREGIEEAINTKLRDSFSQDQVLELVQFRKNNSSLFSKGWHNFSLKLMMELIPELYETSEEQMTILTRLGKQKSKETSKRTKYIDEKEVTEEIYNPVVAKSVRQAIKIINEATKKHGIFDNIVIEMARENNEEDAKKDYIKRQKANQDEKYAAMEKAAFQYNGKKELPDNIFHGHKELATKIRLWHQQGEKCLYTGKSIPISDLIHNQYKYEIDHILPLSLSFDDSLSNKVLVLATANQEKGQRTPFQALDSMDDAWSYREFKSYVKESKLLGNKKKEYLLTEEDISKIEVKQKFIERNLVDTRYSSRVVLNALQDFYKNHNFDTTISVVRGQFTSQLRRKWGLEKSRETYHHHAVDALIIAASSQLRLWKKQNNPLIAYKEGQFVDSQTGEIISLTDDEYKELVFKAPYDHFVDTLSSKTFEDSILFSYQVDSKFNRKISDATIYATRKAKLDKEKKEYTYTLGKIKDIYSLGTKTPSKTGFYKFLDLYNKDKSQFLMFQKDRKTWDEVIEKIMEQYRPFKEYDKAGKLVDFNPFEKYRQENGPIRKYSKKGNGPEIKSLKYYDILLGKHKNITPEGSRNTVALLSLNPWRTDVYYNMETKKYEFLGLKYADLPFEEGGAYGISTETYNELREKEGIGKNSEFKFTLYKNDLILIKDTETNCQQFFRFWSRTGKDNPKSFEKHKIELKPYEKAKFEKGEELEVLGKVPPSSNQFQKNMQIENLSIYKVKTDVLGNKHFIKKEGDKPKLKFKRPAATKKAGQAKKKKGSYPYDVPDYAYPYDVPDYAYPYDVPDYA

SEQ ID NO: 24 - protein, 1109 aa, synthetic construct
MPKKKRKVGTMKEKYILGLALGITSVGYGIINFETKKIIDAGVRLFPEANVDNNEGRRSKRGSRRLKRRRIHRLDRVKSLLTEYNLINREQIPTSNNPYQIRVKGLSEILSKDELAIALLHLAKRRGIHNINVSSEDEDASNELSTKEQINRNNKLLKNKYVCEVQLQRLKEGQIRGEKNRFKTTDILKEIDQLLKVQKDYHNLDIDFINQYKEIVETRREYFEGPGQGSPFGWNGDLKKWYEMLMGHCTYFPQELRSVKYAYSADLFNALNDLNNLIIQRNDSEKLEYHEKYHIIENVFKQKKKPTLKQIAKEIGVNPEDIKGYRITKSGTPQFTEFKLYHDLKSIVFDKSILENEAILDQIAEILTIYQDEESIKEELNKLPEILNEQDKAEIAKLTGYNVTHRLSLKCIHLINEELWQTSRNQMEIFNYLNIKPNKVDLSEQNKIPKDLVDEFILSPVVKRTFIQSINVINKVIEKYGIPEDIIIELARENNSDDRKKFINNLQKKNEATRKRINEIIGQTGNQNGKRIVEKIRLHDQQEGKCLYSLESIPLMDLLNNPQNYEVDHIIPRSVAFDNSIHNKVLVKQIENSKKGNRTPYQYLNSSDANLSYNQFKQHILNLSKSKDRISKKKKDYLLEERDINKFEVQKEFINRNLVDTRYATRELTSYLKAYFSANNMDVKVKTINGSFTNHLRKVWRFDKYRNHSYKHHAEDALIIANADFLFKENKKLQNANKILEKPTIENDTQKVTVEKEEDYNNMFETPKLVEDIKQYRDYKFSHRVDKKPNRQLIKDTLYSTRMKDEHNYIVQTITDIYGKDNTNLKKQFNKNPEKFLMYQNDPKTFEKLSIIMKQYSDEKNPLAKYYEETGEYLTKYSKKNNGPIVKKIKLLGNKVGNHLDVTNKYENSTKKLVKLSIKNYRFDVYLTEKGYKFVTIAYLNVFKKDNYYYIPKDLYQELKAKKKIKDTDQFIASFYKNDLIKLNGDLYKIIGVNSDDRNIIELDYYDIKYKDYCEINNIKGEPRIKKTIGKKTESIEKLTTDVLGNLYLHTTEKAPQLIFKRGLKRPAATKKAGQAKKKKGSYPYDVPDYAYPYDVPDYAYPYDVPDYA

SEQ ID NO: 25 - protein, 1108 aa, synthetic construct
MPKKKRKVGTMKRNYILGLAIGITSVGYGIIDYETRDVIDAGVRLFKEANVENNEGRRSKRGARRLKRRRRHRLQRVKKLLFDYNLLTDHSELSGINPYEARVKGLSQKLSEVEFSAALLHLAKRRGVHNVNEVEEDTGNELSTKEQISRNSKALEEKYVAELQLERLKTDGEVRGPNNRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVIARDENEKLEYYEKFQIIENVFKQKKKPTLKQIAKELLVNEEDIKGYRVTSIGKPEFTNFKIYHDIKGITERKEVLENAELLDQIAEILTIYQSSEDVQEELANLNSELTQEEIEQISNLKGYTGTHNLSLKAINLILDELWHTNDNQMAIFNRLKLVPKKVDLSQQKEIPTTLVDDFILSPVVKRSFIQSIKVINAIIKKYGLPNDIIIELAREKNSKDAQKMINEMQKRNRQMNERIEEIIRTTGKENAKYLIEKIKLHDMQEGKCLYSLESIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRELMNLLRSYFRVNNLDVKVKSINGGFTSFLRRKWKFKKERNKGYKHHAEDALIIANADFIFKEWKKLDKAKKVMENQTVEEKQAESMPEIETEQEYKEIFITPHQIQHIKGFKDYKYSHRVDKKPNRELINDTLYSTRKDDKGNTLIVNNLNGLYDKDNDKLKKLMNKSPEKLLMYHHDPQTYQKLKLIMEQYGDEKNPLYKYYEETGNYLTKYSKKDNGPVIKKIKYYGKNLKAHLDITDDYPNSRNKVVKLSVKPYRFDVYLDNDIYKFVTVKNLDVIKKEDYYEVNSKCYKEAKKLKKISDQAEFIASFYNNDLIKINGELYRVIGVNNDLLNRIEVNMINITYREYLENMNDKRPPRIIKTIASKTQSIKKYSTDILGNLYEVKSKQKPQMIMKGKRPAATKKAGQAKKKKGSYPYDVPDYAYPYDVPDYAYPYDVPDYA

SEQ ID NO: 26 - protein, 1185 aa, synthetic construct
MPKKKRKVGTMTNGKILGLAIGIASVGVGIIEAKTGKVVHANSRLFSAANAENNTERRGFRGSRRLNRRKKHRVKRVRDLFEKHEIVTDFRNLNLSPYELRVKGLTEQLTNEELFAALRTISKRRGISYLDDAEDDSTGSTDYAKSIDENRRLLKNKTPGQIQLERLEKYGQLRGNFTVYDENGEAHRLINVFSTSDYEKEARKILETQADYNKKITAEFIDDYVEILTQKRKYYHGPGNEKSRTDYGRFRTDGTTLENIFGILIGKCNFYPDEYRASKASYTAQEYNFLNDLNNLKVPTETGKLPTEQKESLVEFAKNTATLGPSKLLKEIAKILDCKVDEIKGYREDDKGKPDLHTFEPYRKLKFNLESINIDDLSREVIDKLADILTLNTEREGIEDAIKRNLPNQFTEEQISEIIKVRKSQSTAFNKGWHSFSAKLMNELIPELYATSDEQMTILTRLEKFKVNKKSSKNTKTIDEKEVTDEIYNPVVAKSVRQTIKIINAAVKKYGDFDKIVIEMPRDKNADDEKKFIDKRNKENKKEKDDALKRAAYLYNSSDKLPDEVFHGNKQLETKIRLWYQQGERCLYSGKPIPIHDLVHNSNNFEIDHILPLSLSFDDSLANKVLVYDWANQEKGQKTPYQVIDSMDAAWSFREMKDYVLKQKGLGKKKRDYLLTTENIDKIEVKKKFIERNLIDTRYASRVVLNSLQSALRELCKDTKVSVIRGQFTSQLRRKWKIDKSRETYHHHAVDALIIAASSQLKLWEKQDNLMFIDYGNNQVVDKETGEILSVSDDEYKELVFQPPYQGFVNTISSKGFEDEILFSYQVDSKYNRKVSDATIYSTRKAKIGKDKKEETYVLGKIKDIYSQNGFDTFIKKYNKDKTQFLMYQKDPLTWENVIEVILRDYPTTKKSEDGKNDVKCNPFEEYRRENGLICKYSKKGKGTPIKSLKYYDKKLGNCIDITPEESRNKVILQSINPWRADVYFNPETLKYELMGLKYSDLSFEKGTGKYHISQEKYDAIKEKEGIGKKSEFKFTLYRNDLILIKDTASGEQEIYRFLSRTMPNVNHYAELKPYDKEKFDGGQELMEVFGKVANGGQCLKSLNKSNISIYKVRTDVLGNKYFVKKEGDKPKLNFKNNKKKRPAATKKAGQAKKKKGSYPYDVPDYAYPYDVPDYAYPYDVPDYA

SEQ ID NO: 27 - protein, 1451 aa, synthetic construct
MPKKKRKVGTMKRVNEDYILGLAIGTNSCGWAVTDKKNNLLKLRGKTAIGSHLFEEGHTAADRRGFRTTRRRLKRRKWRLRLLEEIFAEPMAKVDPGFFVRLHQSWVSPLDKDRKKYNAIVFPTAKEDQAFYKHYATIYHLRDELMTQDRQFDLREIFLAIHHIVKYRGNFLQDTPVKDFEASKIEVGPILSHINNAFAEKIVEDQDPIELNVANAADIEDVIRGKDAEKTVYKLDKVKKIAKLLTDSTAKEEKNVAKQIANAIMGYKTQFETILDKEIDKSDKAQWEFKLSDADADDKLDALLPDLDETDQTVVAEIEKLFSAITLSTIVDENKSLSQSMVEKYKKHKKDYKKLKKYINTLQDQTKAKKLLLAYDLYVNNRHGRLLEAKKTFEDKKKKKALTKDEFYKIVKDNLDDSDLAHEIQQEIAADNFMPKQRTNSNGVIPFQLHQIELDKIIANQGKYYPFLAAENPVEDHRKQAPYKLDELVRFRVPYYVGPMITADEQEKTSGKSFAWMVRKEDGQITPWNFEQKVDRQESANKFIKRMTIKDTYLLSEDVLPANSLLYQRFEVLNELNNIRINGSRISVDLKQQIFNDLFEEKKTVTEKSLTSYLKQNLHLPTVEIKGLADPTKFNSSLASYYHLKSLHVFDKELADPQYQKDFEKIIEYSSIFEDKKIFQDKLHAEFKWLTPEQFKAISTWRLQGWGRLSRKLLVELHDTNGQNIMEQLWDSQKNFMQIVTEPDFKDAIAKENQNVTRANGVEEILADAYTSPANKKAIRQVVKVVADVVKAAGGKKPAQFAIEFTRDPDKNPQLSHIRGTKLLKAYQETAGELVDQKLTDSLKEAMTSRKLLKDKYFLYFMQAGRDAYTGQKINIDEVSTNYQIDHILPQSFIKDDSFDNRVLTATPLNAEKSDDVPYKRFANNYVSDMKMTVGEMWKHWQKAGIINKHKLGNLLLDPDRLNKFQKSGFINRQLVETSQIIKLVSVILQNKYPDAEIITVKAGDNSALRQRLNLYKSRDVNDYHHAIDAYLSIICGNFLYQVYPKYRPYFVYGKYKKFSQDPDLQKEVIKHFKGFTFMWPLLQKDNSERKAPEKIKENNSDRIVFYKHPDIFDKLRKAYNYKYMLVSRETTTENSGLFDVTIYPRGERDLAKTRKLIPKSNGLDPKIYGGYSGNTDAYMVIVKIDKGKESIYKVIGVPMRALASLNRAKKQGNYKEELHQVLEPQIMFDKNGKPKRSVKGFRIIKDHVPFKQVVLDGDKKFMLNSSTYEINAKQLTLTPETMRIVTDNLKKGEDQDQLLVKAYDEILQKVDQYLPLFDVNKFRNSLHLGRAKFLDLAVNDKKITLTNILNGLHDNLVTPDLKNIGIKTPLGKLQVPSGIVLSSEAILIFQSPTGLFEKRVRIADLKRPAATKKAGQAKKKKGSYPYDVPDYAYPYDVPDYAYPYDVPDYA

SEQ ID NO: 28 - protein, 1177 aa, synthetic construct
MPKKKRKVGTMSNGKILGLAIGIASVGVGVIDAQTGEIIHASSRIFPSANAANNAERRTFRGSRRLIRRKKHRIKRLDDLFNDFHINLDGEMSTDNPYVLRVKGLSQKLTVEELYISIKNIMKRRGISYLDDAESDNEAGRSDYAKAIERNRQLLTSKTPGEIQLERLEKYGQLRGNFTIIDEEGQSQQIINVFSTSDYVKEVEKILDCQKMYHKFISDEFCDKLIELLREKRKYYVGPGNEKSRTDYGIYRTDGTTLENLFGILIGKCTFYPDQYRSSRASYTAQEFNFLNDLNNLTVPTETKKLSQEQKEFLVNYAKETSVLGAGKILQQIAKLADCKVEDIRGYRLDNKDKPELHTFETYRAMKGLVPLVDIGVLSREQLDILADILTLNTDFEGIREALKKQLPNVFDEKQVKGLASFRKSKSQLFAKGWHNLSQKIMLEVIPELYATSDEQMTILTRLGKFEKSSVAEYPSSINVDEITDEIYNPVVAKSIRQTIKIINASIKKWDEFDQIVIEMPRDRNEDEEKKRIADGQKANAKEKADSILRAAELYCAGKVLPDYVYNGHNQLATKIRLWYQQGERCIYTGQPISIHDLIHNQNQYEIDHILPLSLTFDDSLSNKVLVLATANQEKAQRTPYNYLKSATSAWSYREFKDYVTKRKGIGKKKCEYLTFEEDINGFEVRSKFIQRNLVDTRYASKVILNALQDYFKISGIQTKVSVVRGQFTSQLRHKWGIEKTRETYHHHAVDALIIAASSQLRLWKKQESPLVVDYQEGRQVDLETGEILELTDEQYKELVYQPPYQGFVNTISSSAFDNEILFSYQVDSKVNRKISDATIYATRNAQLGKDKTEGIYVLGKIKDIYTQAGYEAFLKRYTKDKTSFLMYHKDLDTWEKVIEIILRDYREYDEKGKEIGNPFERYRRENGYVKKYSRKGNGTAIKSLKYYDNKLGNHIDITPENSRNAVVLQSLKPWRTDVYFNKETGKYEFLGIKYSDLSFEKGTGEYGISQEKYDSIKIAEGVAKKSIFKFTLYKQDLLFIKDIENNFGKLLRFTSKNDTSKHYVELKPYDKNKFGTEEPLLPVLGNVAKSGQCIKGLNKSNISIYKVRTDILGYRHFIKQEGEHPQLKFKKKRPAATKKAGQAKKKKGSYPYDVPDYAYPYDVPDYAYPYDVPDYA

SEQ ID NO: 29 - protein, 1649 aa, synthetic construct
MPAAKRVKLDGSEVEFSHEYWMRHALTLAKRARDEREVPVGAVLVLNNRVIGEGWNRAIGLHDPTAHAEIMALRQGGLVMQNYRLYDATLYVTFEPCVMCAGAMIHSRIGRVVFGVRNAKTGAAGSLMDVLHHPGMNHRVEITEGILADECAALLCRFFRMPRRVFNAQKKAQSSTDGSSGSETPGTSESATPESSGPKKKRKVGTMEKSYSIGLAIGTNSVGWSVITDDYKVPAKKMRVLGNTDKKYIKKNLLGALLFDSGETAEATRLKRTARRRYTRRKNRLRYLQEIFAKEMAKVDESFFQRLDESFLTDDDKTFDSHPIFGNKAEEDAYHQKFPTIYHLRKHLADSQEKADLRLIYLALAHMIKFRGHFLYDNFNDDNFDWRNIDIQKRYEEFIETYDSTLGESYLADISVDAASILEEKVSKTERLENLLKYYPTEKKTTFFGNLIKLILGQQAKFKAIFNLEDEISLQFATPTYDEDLEELLGKIDNGDSYSELFVAAQNLYNTILLASFLKTDNKSAKAPLSTSMIERYENHKKDLAKLKDFVKKNCPDQYHDIFRDKSKNGYAGYIDNGVSQNDFYTFIGKCLEESLKKDKGAQYFLDKIDRDDFLRKQRTFDNGAFPYQIHLQEMHAILRRQGDYYPFLKENQDKIEKILTFRIPYYVGPLARKDSRFAWANYRSDEAITPWNFDEVVDKEKSAEKFITRMTLNDLYLPEEKVLPKHSLIYETFTVYNELTNIKYVNDQGNAIHFDSELKEKIFNQLFKENRKVSKKTLIDFLNNSEGIYTDKLVGIDEEVKYLNASLGTYHDLKKILESFMDDEINEKIIEDIIQTLTLFEDIEMKRQRLQKYDDIFTPKQLKELARRNYTGWGRLSYKLINGIRNKENNKTILDYLKNGNRNFMQLINDDRLSFKQIIIDARKIEKLDNIESVVYNLPGSPAIKKGILQSIKIVDELVKVMGHNPDNIVIEMARENQTTNQGKNRSQQRLKRLQDSMSNFKDSSISLKDVDNSDLQNDRLFLYYIQNGKDMYTGEELDIDHLSDYDIDHIIPQSFIKDNSIDNRVLTSSAKNRGKSDDVPGRDVVLKMKPFWKKLYDVKLISKRKFDNLTKSEHGGLTESDKAGFIKRQLVETRQITKYVAQILDGRFNTKRDDNNKVIRDVKVITLKSSLVSQFRKDFGFYKVREINDYHHAHDAYLNAVVGTAILKKYPKLAPEFVYGEYKKCDVRKLIAKSGDKSEIGKATAKYFFYSNLMNFFKRVIRYSNGMIVVRPVIEYSKDTGEIAWDKEKDFKTVCKVLSCPQVNIVKKVEKQSHGLDRGKPKGFYNANPSPKPKKGSKVNLVPIKANLNPKNYGGYAGISNSYAVLVDATIEKGAKKKLTRIQEFQGISIIDREKYEKNKVEFLKGLGYKEIYSIITLPKYSLFELADGSRRMLASILSTNNKRGEIHKGNELVLPAKYIPLLYHANRIHNTFETGHREYVEKHIAEFKEIAEIILEFNNKYVNAKKNSSIIEKALESFDSFSLDEICDSFVGKLKKNNTKKNSGLFELVSLGSASDFEFLETKVPRYRDYTPSSLLNATLIHQSITGLYETRIDLSKLGEEKRPAATKKAGQAKKKKGSYPYDVPDYAYPYDVPDYAYPYDVPDYA

SEQ ID NO: 30 - protein, 1587 aa, synthetic construct
MPAAKRVKLDGSEVEFSHEYWMRHALTLAKRARDEREVPVGAVLVLNNRVIGEGWNRAIGLHDPTAHAEIMALRQGGLVMQNYRLYDATLYVTFEPCVMCAGAMIHSRIGRVVFGVRNAKTGAAGSLMDVLHHPGMNHRVEITEGILADECAALLCRFFRMPRRVFNAQKKAQSSTDGSSGSETPGTSESATPESSGPKKKRKVGTMTKDYTIGLAIGTNSVGWAVLTDDYQLMKRKMSVHGNTEKKKIKKNFWGARLFDEGQTAEFRRTKRTNRRRLARRKYRLSKLQDLFAEELCKQDDCFFVRLEESFLVPEEKQYKPASIFPTLEEEKEYYQKYPTIYHLRQKLVDSTEKEDLRLVYLALAHLLKYRGHFLFEGDLDTENTSIEESFRVFLEQYSKQSDQPLIVHQPVLTILTDKLSKTKKVEEILKYYPTEKINSFFAQCLKLIVGNQANFKRIFDLEAEVKLQFSKETYEEDLESLLEKIGDEYLDIFLQAKKVHDAILLSEIISSTVKHTKAKLSSGMVERYERHKADLAKFKQFVKENVPQKSTAFFKDTTKNGYAGYIKGKTTQEEFYKFVKKELSGVVGSEPFLEKIDQETFLLKQRTYTNGVIPHQVHLIELKAIIDQQKQHYPFLEEAGPKIIALFKFRIPYYVGPLAKEQEASSFAWIERKTAEKIHPWNFSEVVDIEKSAMRFIQRMTKQDTYLPTEKVLPKNSLLYQKYMIFNELTKVSYKDERGVKQYFSGDEKQQIFKQLFQKERGKITVKKLQNFLYTHYHIENAQIFGIEKAFNASYSTYHDFMKLAKTNQKAMQEWLEQPEMEPIFEDIVKILTIFEDRQMIKHQLSKYQEVFGEKLLKEFARKHYTGWGRFSAKLIHGIRDRKTNKTILDYLINDDDVPANRNRNLMQLINDEHLSFKEEIAKATVFSKHKSLVDVIQDLPGSPAIKKGIWQSLKIVEELIAIIGYKPKNIVIEMARENQKTHRTKPRLKALENGLKQIGSTLLKEQPTDNKALQKERLYLYYLQNGRDMYTGEPLEIENLHQYEVDHIIPRSFIVDNSIDNKVLVARKQNQKKRDDVPKKQIVNEQRIFWNQLKEAKLISPKKYAYLTKIELTPEDKARFIQRQLVETRQITKHVANILHQSFHQEEEGTDCDGVQIITLKATLTSQFRQTFGLYKVREINPHHHAHDAYLNGFIANVLLKRYPKLAPEFVYGKYVKYSLARENKATAKKEFYSNILKFFESDEPFCDENGEIYWEKSHHLPRIKKVLSSHQVNVVKKVEQQKGGFYKETVNSKEKPDKLIERKNNWDVTKYGGFGSPVIAYAIAFVYAKGKTQKKTRAIEGITIMEQAAFEKDPTTFLKEKGFPQVTEFIKLPKYTLFEFDNGRRRFLASHKESQKGNPFILSDQLVTLLYHAQHYDKITYQESFDYVNTHLSDFSAILTEVLAFAEKYTLADKNIERIQELYEENKYGEISMIAQSFLQLLQFNAIGAPADFKFFGVTIPRKRYTSLTEIWDATIIYQSVTGLYETRIRMGDLWAGEQKRPAATKKAGQAKKKKGSYPYDVPDYAYPYDVPDYAYPYDVPDYA

SEQ ID NO: 31 - protein, 1381 aa, synthetic construct
MPAAKRVKLDGSEVEFSHEYWMRHALTLAKRARDEREVPVGAVLVLNNRVIGEGWNRAIGLHDPTAHAEIMALRQGGLVMQNYRLYDATLYVTFEPCVMCAGAMIHSRIGRVVFGVRNAKTGAAGSLMDVLHHPGMNHRVEITEGILADECAALLCRFFRMPRRVFNAQKKAQSSTDGSSGSETPGTSESATPESSGPKKKRKVGTMTNGKILGLAIGVASVGVGIIEAKTGKVIHANSRLFSAANAENNAERRGFRGARRLTRRKKHRVKRVRDLFEKYDISTDFRNLNLNPYELRVKGLSEQLTNEELFAALRTIAKRRGISYLDDAEDDSTGSSDYAKSIDENRRLLKSMTPGQIQLERLEKYGQLRGNFTVYDENGEAHRLINVFSTSDYKNEARKILETQSNYNKQITDEFIEDYIEILTQKRKYYHGPGNEKSRTDYGRFRTDGTTLENIFGILIGKCSFYPEEYRASKASYTAQEFNFLNDLNNLKVPTETGKLSTEQKEYLVDFAKNSKTLGASKLLKEIAKLVDGDVKDISGYREDSKGKPDLHTFEPYRKLKFNLTTVDIDNLSRDILDKLANILTLNTEREGIEDAINRNLPEQFTKEQISEIVQIRKSQSSAFNKGWHSFSAKLMNELIPELYATSEEQMTILTRLEKFKATKKSSKNTKTIDEKEITDEIYNPVVAKSVRQTIKIINAAVKKYGDFDKIVIEMPRDKNADDEKKFIDKKNKENKKEKDDSLKRAAYLYNGTDKLPDDVFHGNKQLATKIRLWYQQGERCLYSGKPILIQDLVHNSNNFEIDHILPLSLSFDDSLANKVLVYAWTNQEKGQKTPYQVIDSMDAAWSFREMKDYVLKQKGIGKKKREYLLTTENIDKIEVKKKFIERNLVDTRYASRVVLNSLQTALKELGKDTKVSVVRGQFTSQLRRKWKIDKSRETYHHHAVDALIIAASSQLKLWQKHENLMFENYGENQVVNKETGEILSISDDEYKELVFQPPYQGFVNTISSKAFEDEILFSYQVDSKFNRKVSDATIYSTRKAKLGKDKKEETYVLGKIKDIYSQDGFDTFIKRYKKDKTQFLMYQKDPLTWENVIEVILRDYPTTKKSEDGKNDVKCNPFEEYRRENGLICKYSKKGKGTPIKSLKYYDKKLGNHISITPKESKNDVVLQSLNPWRADLYFNPDTLKYELMGLKYSDLSFEKGTGKYHISQEKYDEIKEKEGIGQNSEFKFTLYRNDLILIKDTESGEQEIYRFLSRTMPNVKHYVELKPYDKEKFNGGQELIKSLGEADKVGRCLKGLSKPGISIYKVRTDVLGNKFFVKKEGDKPKLDFKNNKKKRPAATKKAGQAKKKKGSYPYDVPDYAYPYDVPDYAYPYDVPDYA

SEQ ID NO: 32 - protein, 1376 aa, synthetic construct
MPAAKRVKLDGSEVEFSHEYWMRHALTLAKRARDEREVPVGAVLVLNNRVIGEGWNRAIGLHDPTAHAEIMALRQGGLVMQNYRLYDATLYVTFEPCVMCAGAMIHSRIGRVVFGVRNAKTGAAGSLMDVLHHPGMNHRVEITEGILADECAALLCRFFRMPRRVFNAQKKAQSSTDGSSGSETPGTSESATPESSGPKKKRKVGTMNGLVLGLAIGIASVGVGILNKETGEIIHVNSRIFPAATADSNVERRGFRQGRRLGRRKKHRSARLNDLFEEFGFITDFSAVPLNLNPYALRVKGLSEELTNEELFIALKNIIKRRGISYLDDASEDGETASNEYGKAVEENRKLLADKTPGQIQLERFEKYGQVRGDFTVVENGENHRLINVFSTSAYKKEAERILRRQQEFNVRISDEFIEAYLTILTGKRKYYHGPGNEKSRTDYGRFRTDGTTLDNIFGILIGKCTFYPDEYRAAKASYTAQEFNLLNDLNNLTVPTETKKLRPEQKRQIVEYARTAKTLGTPTLLKYIAKLVDGSIDDIKGYRIDKSDKPEMHTFDAYRKMRTLELVDVDILSRETLDDLAYILTLNTESEGILEALNSKMPGTFTKEQIDELIQFRKKNSAVFGKGWHNFSLKLMNELISELYETSEEQMTILTRLGKQRSREISKRTKYIDEKELTDEIYNPVVAKSVRQAIKIINLATKKYGIFDNIVIEMARESNEDDEKKAIQNVQKANEDEKKAAMEKAADLYNGKKELPDSIFHGHKELATKIRLWHQQGERCLYTGKNISIHDLIHNPHQYEIDHILPLSLSFDDGLANKVLVLATANQEKGQRTPFQALDSMDDAWSYIEFKQYVRNSKSLSNKKKDYLLTEEDISKIEVKQKFIERNLVDTRYSSRVVLNTLQEFYKTNDFDTKISVVRGQFTSQLRRKWKIEKSRDTYHHHAVDALIIAASSQLRLWKKQNNPLISYREGQLVDPETGEILSLTDDEYKELVFRPPYDYFVDTLKSKSFEDSILFSYQVDSKYNRKISDATIYGTRKAQLGKDKQEETYVLGKIKDIYSQKGYEDFIKRYKKDTTQFLMYHKDPQTFAKVIEEILKTYPDKELNEKGKEIPCNPFEKYRQENGPIRKYSKKGKGPEIKSLKYYDNKLGNHIDITPVNSQNQVVLQSLKPWRTDVYFNPQTSKYELMGLKYSDLRFEKGSGSYGISPEKYNKVKAKEGVDEDSEFKFTLYKNDLILIKDTETGEQQLFRYGSRNDTSKHYVELKPYEKAKFEGNQQLMNLLGTVAKGGQCLKGINKPNLSIYKVKTDVLGNKHFIKKEGDQPQLNFKKKIKRPAATKKAGQAKKKKGSYPYDVPDYAYPYDVPDYAYPYDVPDYA

SEQ ID NO: 33 - protein, 1304 aa, synthetic construct
MPAAKRVKLDGSEVEFSHEYWMRHALTLAKRARDEREVPVGAVLVLNNRVIGEGWNRAIGLHDPTAHAEIMALRQGGLVMQNYRLYDATLYVTFEPCVMCAGAMIHSRIGRVVFGVRNAKTGAAGSLMDVLHHPGMNHRVEITEGILADECAALLCRFFRMPRRVFNAQKKAQSSTDGSSGSETPGTSESATPESSGPKKKRKVGTMNNSYILGLAIGITSVGYGIIEYETRDVIDAGVRLFKEANVENNEGRRSKRGARRLKRRRRHRLQRVKKMLFDYKLLNEDSEISGINPYEARVKGLSEKLSDEEFSAALLHLAKRRGVHNVSDVEEDTGNELSTKEQIARNNKALEDKYVAELQLERLKEQGEVRGAANRFKTSDYIKEAKQLLKTQSDYHKIDETFIETYISLLETRRTYYEGPGEGSPFGWKDIKEWYEMLMGHCTYFPEELRSVKYAYNADLYNALNDLNNLVIARDENEKLEYYEKFQIIENVFKQKKKPTLKQIAKEILVNEEDIKGYRVTSTGKPEFTNFKIYHDIKGITSRKEILENADLLNQIAEILTIYQSAEDIQEELAELEPKLTQEEIEQISNLTGYTGTHRLSLKAINLILDELWNTSDNQMTIFNRLKLVPKKVDLSQQKEIPTSLVDDFILSPVVKRSFIQSIKVINAIIKKFGLPKDIIIELAREKNSKEAQKFINEMQKRNRQTNERIEKIIKETGKEKAKFLIEKIKLHDMQEGKCLYSLESIPLEDLLNNPYHYEVDHIIPRSVSFDNSFNNKVLVKQEENSKKGNRTPFQYLSSSDAKISYETFKKHILNLSKGKGRVSKKKKEYLLEERDINRFSVQKDFINRNLVDTRYATRELMNLLRSYFRVNDLDVKVKSINGGFTSFLRTKWKFKKERNQGYKHHAEDALVIANADFIFKEWKKLDTTNKVMENQTVEEQQAENMPGIETDDEYKEIFVIPRQIQSIKDFKDYKYSHRVDKKPNRELVNDTLYSTRKDDKGNTLIINNIKGLYDKDNDKLKNLIKKSPEKLLMYHHDPQTYQKLKTIMEQYSNEKNPLYKYHEETGNYLTKYSKKDNGPIIKKVKYYGKKLNAHLDITNDYSNSQNKIVKLSLKPYRFDVYLDNGGYKFVTVKNLDVIKKEGFFKIDSNAYEKAKSEKKIDENAVFIASFYNNDLIKIDGELYRIVGVNNDTRNVVELNMIPITYKEYLENINDKRTPRILKTISQKTYSIEKYSTDILGNLYKVKSKKKPQMIMKGKRPAATKKAGQAKKKKGSYPYDVPDYAYPYDVPDYAYPYDVPDYA

SEQ ID NO: 34 - protein, 1643 aa, synthetic construct
MPAAKRVKLDGSEVEFSHEYWMRHALTLAKRARDEREVPVGAVLVLNNRVIGEGWNRAIGLHDPTAHAEIMALRQGGLVMQNYRLYDATLYVTFEPCVMCAGAMIHSRIGRVVFGVRNAKTGAAGSLMDVLHHPGMNHRVEITEGILADECAALLCRFFRMPRRVFNAQKKAQSSTDGSSGSETPGTSESATPESSGPKKKRKVGTMENKNYSIGLAIGTNSVGWAVITDDYKVPSKKMKVFGNTDKHFIKKNLIGALLFDEGATAEDRRLKRTARRRYTRRKNRLRYLQEIFSEEISKLDSSFFHRLDDSFLVPKDKRGSKYPIFATLEEEKEYHKKFPTIYHLRKHLADSKEKTDLRLIYLALAHMIKYRGHFLYEESFDIKNNDIQKIFNEFISIYDNTFEGSSLSGQNAQVEAIFTDKISKSAKRERVLKLFSDEKSTSLFSEFLKLIVGNQADFKKHFDLEEKAPLQFSKDTYDEDLENLLGQIGDGFTDLFLVAKKLYDAILLSGILTVTDPSTKAPLSASMIERYESHQKDLAALKQFIKNNLPKRYNEVFSDQSKDGYAGYIDGKTTQEAFYKYIKNLLSKFEGADYFLDKIEREDFLRKQRTFDNGSIPHQIHLQEMNAILRRQGEHYPFLKENREKIEKILTFRIPYYVGPLARGNRDFAWLTRNSDQAIRPWNFEEIVDKASSAEEFINKMTNYDLYLPEEKVLPKHSLLYETFAVYNELTKVKFIAEGLRDYQFLDSGQKKQIVNQLFKEKRKVTEKDIIHYLHNVDGYDGIELKGIEKQFNASLSTYHDLLKIIKDKEFMDDPKNEEILENIVHTLTIFEDREMIKQRLAQYDSIFDEKVIKALTRRHYTGWGKLSAKLINGICDKQTGDTILDYLIDDGKINRNFMQLINDDGLSFKEIIQKAQVVGKTDDVKQVVQELPGSPAIKKGILQSIKIVDELVKVMGYAPESIVIEMARENQTTARGKKNSQQRYKRIEDSLKNLAPGLDSNILKENPTDNIQLQNDRLFLYYLQNGKDMYTGKPLDIDQLSSYDIDHIIPQAFIKDDSIDNRVLTSSKDNRGKSDNVPSLEVVQKRKAFWQQLLDSKLISERKFNNLTKAERGGLDERDKVGFIRRQLVETRQITKHVAQILDASFNTEVNEKNQKIRTVKIITLKSNLVSNFRKEFELYKVREINDYHHAHDAYLNAVVAKAILKKYPKLEPEFVYGDYQKYDLKRYISRFKPSKEIEKATEKYFFYSNLLNFFKEEVHYADGIIVKRENIEYSKDTGEIAWNKEKDFATIKKVLSYPQVNIVKKTEIQTHGLDRGKPKGLFNSNPSPKPSEDSKENLVPIKQGLDPRKYGGYAGISNSYAVLVKAIVEKGAKKQQKTILEFQGISILDKINFENNKENYLLKKRYIEILSTITLPKYSLFEFPDGTRRRLASILSTNNKRGEIHKGNELVLPGKYTTLLYHAKNINKKLEPEHLEYVEKHRNDFAKLLECVLNFNDKYVGALKNGERIRQAFTDWETVDIEKLCFSFIGPENSKNAGLFELTSQGSASDFEFLGVKIPRYRDYAPSSLLKATLIHQSITGLYETRIDLSKLGEDKRPAATKKAGQAKKKKGSYPYDVPDYAYPYDVPDYAYPYDVPDYA

SEQ ID NO: 35 - protein, 1390 aa, synthetic construct
MPAAKRVKLDGSEVEFSHEYWMRHALTLAKRARDEREVPVGAVLVLNNRVIGEGWNRAIGLHDPTAHAEIMALRQGGLVMQNYRLYDATLYVTFEPCVMCAGAMIHSRIGRVVFGVRNAKTGAAGSLMDVLHHPGMNHRVEITEGILADECAALLCRFFRMPRRVFNAQKKAQSSTDGSSGSETPGTSESATPESSGPKKKRKVGTMSDLVLGLAIGIGSVGVGILNKVTGEIIHKNSRIFPAAQAENNLERRTNRQGRRLTRRKKHRRVRLNHLFEESGLITDFTKVSINLNPYQLRVKGLTAELSNEELFIALKNMVKHRGISYLDDASDDGNSSVGDYAQIVKENSKQLETKTPGQIQLERYQKYGQLRGDFTVEEDGRKHRLINVFPTSAYHAEALRILQTQQEFNPQITDEFINSYLEILTGKRKYYHGPGNEKSRTDYGKYTTKKDAQGQYITLNNIFGILIGKCTFYPEEYRAAKASYTAQEFNLLNDLNNLTVPTETKKLSEEQKYQIITYVKNEKAMGPAKLFKYIAKLLSCDVADIKGYRIDKSDKAEIHTFEAYRKMKTLETLDVKKMAREELDKLAYVLTLNTEREGIQEALDHEFADGTFSQEQVDELVQFRKANSSIFGKGWHSFSVKLMMELIPELYATSEEQMTILTRLGKQKTTSSSNKTKYIDEKQLTEEIYNPVVAKSVRQAIKIVNAAIKKYGDFDNIVIEMARETNEDDEKKAIQKIQKANKAEKDAAMRKAANQYNGKAELPHSVFHGHKQLATKIRLWHQQGERCLYTGKTISIHDLINNPNQFEIDHILPLSITFDDSLANKVLVYATANQEKGQRTPYQALDSMDDAWSFRELKAFVRESKALSNKKKEYLLTEEDISKFDVRKKFIERNLVDTRYASRVVLNALQEHFRTHKIDTKVSVVRGQFTSQLRRHWGIEKTRDTYHHHAVDALIIAASSQLNLWKKQKNTLVSYSEDQLLDIETGELISDDEYKESVFKAPYQHFVDTLKSKEFEDSILFSYQVDSKFNRKISDATIYATRKAKLDKEKKEYTYTLGKIKDIYALGTKTPSKTGFYKFLDLYKKDKSQFLMYQKDRRTWDEVIEKILEQYRPFKEKDKNGKEVDFNPFEKYRIENGPIRKYSRKGNGPEIKSLKYYDNLLGRFVDITPSESKNPVALLSLNPWRTDVYYNTETRKYEFLGLKYADLCFEKGGSYGISKVKYNKIREKEGIGKNSEFKFTLYKNDLILIKDTETNRQQIFRFWSRTGKDNPKSFEKHKLELKPYEKTRFEKGEELKVLGKVPPSSNRLQKNMQIENLSIYKVRTDVLGNQHIIKNEGDKPKLDFKRPAATKKAGQAKKKKGSYPYDVPDYAYPYDVPDYAYPYDVPDYA

SEQ ID NO: 36 - protein, 1373 aa, synthetic construct
MPAAKRVKLDGSEVEFSHEYWMRHALTLAKRARDEREVPVGAVLVLNNRVIGEGWNRAIGLHDPTAHAEIMALRQGGLVMQNYRLYDATLYVTFEPCVMCAGAMIHSRIGRVVFGVRNAKTGAAGSLMDVLHHPGMNHRVEITEGILADECAALLCRFFRMPRRVFNAQKKAQSSTDGSSGSETPGTSESATPESSGPKKKRKVGTMNGLVLGLAIGIASVGVGILEKNSGKIIHANSRIFPAATADNNVERRKNRQARRLHRRKKHRGVRLQDIFEDYGLLTDFSKVSINLNPYRLRVDGLDQQLTNEELFIALKNIVKRRGISYLDDASEDGGTVSSDYGKAVEENRKLLAEKTPGQIQLERFEKYGQVRGDFTVVENGEKRRLINVFSTSAYRKEAERILRKQQEFNSKITDEFIEDCLKILTGKRKYYHGPGNEKSRTDYGRFRTDGTTLDNIFGILIGKCTFYPNEYRASKASHTAQEFNLLNDLNNLTVPTETKKLSEEQKKVIVEYAKEAKTLGASTLLKYIAKMIDASVDQISGYRVDVNNKPEMHTFEVYRKMQSLETISVGELSRNVLDELAHILTLNTEREGIEEAINTKLKDSFSQDQVLELVQFRKNNSSLFSRGWHNFSLKLMMELIPELYETSEEQMTILTRLGKQKSKETSKRTKYIDEKELTEEIYNPVVAKSVRQAIKIINEATKKYGIFDNIVIEMARENNEEDAKKDYIKRQKANQDEKNAAMEKAAFQYNGKKELPDNIFHGHKELATKIRLWHQQGEKCLYTGKNIPISDLIHNQYKYEIDHILPLSLSFDDSLSNKVLVLATANQEKGQRTPFQALDSMDDGWSYREFKSYVKESKLLGNKKKEYLLTEEDISKIEVKQKFIERNLVDTRYSSRVVLNALQDFYKAHKFDTTISVVRGQFTSQLRRKWGLEKSRETYHHHAVDALIIAASSQLRLWKKQNNPLIAYKEGQFVDSETGEILSLSDDEYKELVFKAPYDHFVDTLRSKTFEDSILFSYQVDSKYNRKISDATIYATRKAKLDKDKSEETYVLGKIKDIYSQAGYDAFIKIYNKDKSKFLMYHKDPQTFEKVIEEILRTYPSKELNDKNKEIPCNPFEKYRQENGPIRKYSKKGNGPEIKCLKYYDNKLGNYIDITPDGSDNQVVLQSIAPWRTDVYYNHKTGKYEFLGLKYSDLYFEKGTGKYKISKEKYDNIKKIEGVVETSEFKFTLYKNDLILIKDVEKGQEQLFRFLSRNNKGKHQVQLKPMNKSDFEKGEKLIDIFGTVPNSTTQCVKGLNKSNISIFKVKTDVLGKKHIIKKEGDEPKLKFKRPAATKKAGQAKKKKGSYPYDVPDYAYPYDVPDYAYPYDVPDYA

SEQ ID NO: 37 - protein, 1373 aa, synthetic construct; identical to SEQ ID NO: 36

SEQ ID NO: 38 - protein, 1305 aa, synthetic construct
MPAAKRVKLDGSEVEFSHEYWMRHALTLAKRARDEREVPVGAVLVLNNRVIGEGWNRAIGLHDPTAHAEIMALRQGGLVMQNYRLYDATLYVTFEPCVMCAGAMIHSRIGRVVFGVRNAKTGAAGSLMDVLHHPGMNHRVEITEGILADECAALLCRFFRMPRRVFNAQKKAQSSTDGSSGSETPGTSESATPESSGPKKKRKVGTMKEKYILGLALGITSVGYGIINFETKKIIDAGVRLFPEANVDNNEGRRSKRGSRRLKRRRIHRLDRVKSLLTEYNLINREQIPTSNNPYQIRVKGLSEILSKDELAIALLHLAKRRGIHNINVSSEDEDASNELSTKEQINRNNKLLKNKYVCEVQLQRLKEGQIRGEKNRFKTTDILKEIDQLLKVQKDYHNLDIDFINQYKEIVETRREYFEGPGQGSPFGWNGDLKKWYEMLMGHCTYFPQELRSVKYAYSADLFNALNDLNNLIIQRNDSEKLEYHEKYHIIENVFKQKKKPTLKQIAKEIGVNPEDIKGYRITKSGTPQFTEFKLYHDLKSIVFDKSILENEAILDQIAEILTIYQDEESIKEELNKLPEILNEQDKAEIAKLTGYNVTHRLSLKCIHLINEELWQTSRNQMEIFNYLNIKPNKVDLSEQNKIPKDLVDEFILSPVVKRTFIQSINVINKVIEKYGIPEDIIIELARENNSDDRKKFINNLQKKNEATRKRINEIIGQTGNQNGKRIVEKIRLHDQQEGKCLYSLESIPLMDLLNNPQNYEVDHIIPRSVAFDNSIHNKVLVKQIENSKKGNRTPYQYLNSSDANLSYNQFKQHILNLSKSKDRISKKKKDYLLEERDINKFEVQKEFINRNLVDTRYATRELTSYLKAYFSANNMDVKVKTINGSFTNHLRKVWRFDKYRNHSYKHHAEDALIIANADFLFKENKKLQNANKILEKPTIENDTQKVTVEKEEDYNNMFETPKLVEDIKQYRDYKFSHRVDKKPNRQLIKDTLYSTRMKDEHNYIVQTITDIYGKDNTNLKKQFNKNPEKFLMYQNDPKTFEKLSIIMKQYSDEKNPLAKYYEETGEYLTKYSKKNNGPIVKKIKLLGNKVGNHLDVTNKYENSTKKLVKLSIKNYRFDVYLTEKGYKFVTIAYLNVFKKDNYYYIPKDLYQELKAKKKIKDTDQFIASFYKNDLIKLNGDLYKIIGVNSDDRNIIELDYYDIKYKDYCEINNIKGEPRIKKTIGKKTESIEKLTTDVLGNLYLHTTEKAPQLIFKRGLKRPAATKKAGQAKKKKGSYPYDVPDYAYPYDVPDYAYPYDVPDYA

SEQ ID NO: 39 - protein, 1304 aa, synthetic construct
MPAAKRVKLDGSEVEFSHEYWMRHALTLAKRARDEREVPVGAVLVLNNRVIGEGWNRAIGLHDPTAHAEIMALRQGGLVMQNYRLYDATLYVTFEPCVMCAGAMIHSRIGRVVFGVRNAKTGAAGSLMDVLHHPGMNHRVEITEGILADECAALLCRFFRMPRRVFNAQKKAQSSTDGSSGSETPGTSESATPESSGPKKKRKVGTMKRNYILGLAIGITSVGYGIIDYETRDVIDAGVRLFKEANVENNEGRRSKRGARRLKRRRRHRLQRVKKLLFDYNLLTDHSELSGINPYEARVKGLSQKLSEVEFSAALLHLAKRRGVHNVNEVEEDTGNELSTKEQISRNSKALEEKYVAELQLERLKTDGEVRGPNNRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVIARDENEKLEYYEKFQIIENVFKQKKKPTLKQIAKELLVNEEDIKGYRVTSIGKPEFTNFKIYHDIKGITERKEVLENAELLDQIAEILTIYQSSEDVQEELANLNSELTQEEIEQISNLKGYTGTHNLSLKAINLILDELWHTNDNQMAIFNRLKLVPKKVDLSQQKEIPTTLVDDFILSPVVKRSFIQSIKVINAIIKKYGLPNDIIIELAREKNSKDAQKMINEMQKRNRQMNERIEEIIRTTGKENAKYLIEKIKLHDMQEGKCLYSLESIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRELMNLLRSYFRVNNLDVKVKSINGGFTSFLRRKWKFKKERNKGYKHHAEDALIIANADFIFKEWKKLDKAKKVMENQTVEEKQAESMPEIETEQEYKEIFITPHQIQHIKGFKDYKYSHRVDKKPNRELINDTLYSTRKDDKGNTLIVNNLNGLYDKDNDKLKKLMNKSPEKLLMYHHDPQTYQKLKLIMEQYGDEKNPLYKYYEETGNYLTKYSKKDNGPVIKKIKYYGKNLKAHLDITDDYPNSRNKVVKLSVKPYRFDVYLDNDIYKFVTVKNLDVIKKEDYYEVNSKCYKEAKKLKKISDQAEFIASFYNNDLIKINGELYRVIGVNNDLLNRIEVNMINITYREYLENMNDKRPPRIIKTIASKTQSIKKYSTDILGNLYEVKSKQKPQMIMKGKRPAATKKAGQAKKKKGSYPYDVPDYAYPYDVPDYAYPYDVPDYA

SEQ ID NO: 40 - protein, 1381 aa, synthetic construct
MPAAKRVKLDGSEVEFSHEYWMRHALTLAKRARDEREVPVGAVLVLNNRVIGEGWNRAIGLHDPTAHAEIMALRQGGLVMQNYRLYDATLYVTFEPCVMCAGAMIHSRIGRVVFGVRNAKTGAAGSLMDVLHHPGMNHRVEITEGILADECAALLCRFFRMPRRVFNAQKKAQSSTDGSSGSETPGTSESATPESSGPKKKRKVGTMTNGKILGLAIGIASVGVGIIEAKTGKVVHANSRLFSAANAENNTERRGFRGSRRLNRRKKHRVKRVRDLFEKHEIVTDFRNLNLSPYELRVKGLTEQLTNEELFAALRTISKRRGISYLDDAEDDSTGSTDYAKSIDENRRLLKNKTPGQIQLERLEKYGQLRGNFTVYDENGEAHRLINVFSTSDYEKEARKILETQADYNKKITAEFIDDYVEILTQKRKYYHGPGNEKSRTDYGRFRTDGTTLENIFGILIGKCNFYPDEYRASKASYTAQEYNFLNDLNNLKVPTETGKLPTEQKESLVEFAKNTATLGPSKLLKEIAKILDCKVDEIKGYREDDKGKPDLHTFEPYRKLKFNLESINIDDLSREVIDKLADILTLNTEREGIEDAIKRNLPNQFTEEQISEIIKVRKSQSTAFNKGWHSFSAKLMNELIPELYATSDEQMTILTRLEKFKVNKKSSKNTKTIDEKEVTDEIYNPVVAKSVRQTIKIINAAVKKYGDFDKIVIEMPRDKNADDEKKFIDKRNKENKKEKDDALKRAAYLYNSSDKLPDEVFHGNKQLETKIRLWYQQGERCLYSGKPIPIHDLVHNSNNFEIDHILPLSLSFDDSLANKVLVYDWANQEKGQKTPYQVIDSMDAAWSFREMKDYVLKQKGLGKKKRDYLLTTENIDKIEVKKKFIERNLIDTRYASRVVLNSLQSALRELCKDTKVSVIRGQFTSQLRRKWKIDKSRETYHHHAVDALIIAASSQLKLWEKQDNLMFIDYGNNQVVDKETGEILSVSDDEYKELVFQPPYQGFVNTISSKGFEDEILFSYQVDSKYNRKVSDATIYSTRKAKIGKDKKEETYVLGKIKDIYSQNGFDTFIKKYNKDKTQFLMYQKDPLTWENVIEVILRDYPTTKKSEDGKNDVKCNPFEEYRRENGLICKYSKKGKGTPIKSLKYYDKKLGNCIDITPEESRNKVILQSINPWRADVYFNPETLKYELMGLKYSDLSFEKGTGKYHISQEKYDAIKEKEGIGKKSEFKFTLYRNDLILIKDTASGEQEIYRFLSRTMPNVNHYAELKPYDKEKFDGGQELMEVFGKVANGGQCLKSLNKSNISIYKVRTDVLGNKYFVKKEGDKPKLNFKNNKKKRPAATKKAGQAKKKKGSYPYDVPDYAYPYDVPDYAYPYDVPDYA

SEQ ID NO: 41 - protein, 1647 aa, synthetic construct
MPAAKRVKLDGSEVEFSHEYWMRHALTLAKRARDEREVPVGAVLVLNNRVIGEGWNRAIGLHDPTAHAEIMALRQGGLVMQNYRLYDATLYVTFEPCVMCAGAMIHSRIGRVVFGVRNAKTGAAGSLMDVLHHPGMNHRVEITEGILADECAALLCRFFRMPRRVFNAQKKAQSSTDGSSGSETPGTSESATPESSGPKKKRKVGTMKRVNEDYILGLAIGTNSCGWAVTDKKNNLLKLRGKTAIGSHLFEEGHTAADRRGFRTTRRRLKRRKWRLRLLEEIFAEPMAKVDPGFFVRLHQSWVSPLDKDRKKYNAIVFPTAKEDQAFYKHYATIYHLRDELMTQDRQFDLREIFLAIHHIVKYRGNFLQDTPVKDFEASKIEVGPILSHINNAFAEKIVEDQDPIELNVANAADIEDVIRGKDAEKTVYKLDKVKKIAKLLTDSTAKEEKNVAKQIANAIMGYKTQFETILDKEIDKSDKAQWEFKLSDADADDKLDALLPDLDETDQTVVAEIEKLFSAITLSTIVDENKSLSQSMVEKYKKHKKDYKKLKKYINTLQDQTKAKKLLLAYDLYVNNRHGRLLEAKKTFEDKKKKKALTKDEFYKIVKDNLDDSDLAHEIQQEIAADNFMPKQRTNSNGVIPFQLHQIELDKIIANQGKYYPFLAAENPVEDHRKQAPYKLDELVRFRVPYYVGPMITADEQEKTSGKSFAWMVRKEDGQITPWNFEQKVDRQESANKFIKRMTIKDTYLLSEDVLPANSLLYQRFEVLNELNNIRINGSRISVDLKQQIFNDLFEEKKTVTEKSLTSYLKQNLHLPTVEIKGLADPTKFNSSLASYYHLKSLHVFDKELADPQYQKDFEKIIEYSSIFEDKKIFQDKLHAEFKWLTPEQFKAISTWRLQGWGRLSRKLLVELHDTNGQNIMEQLWDSQKNFMQIVTEPDFKDAIAKENQNVTRANGVEEILADAYTSPANKKAIRQVVKVVADVVKAAGGKKPAQFAIEFTRDPDKNPQLSHIRGTKLLKAYQETAGELVDQKLTDSLKEAMTSRKLLKDKYFLYFMQAGRDAYTGQKINIDEVSTNYQIDHILPQSFIKDDSFDNRVLTATPLNAEKSDDVPYKRFANNYVSDMKMTVGEMWKHWQKAGIINKHKLGNLLLDPDRLNKFQKSGFINRQLVETSQIIKLVSVILQNKYPDAEIITVKAGDNSALRQRLNLYKSRDVNDYHHAIDAYLSIICGNFLYQVYPKYRPYFVYGKYKKFSQDPDLQKEVIKHFKGFTFMWPLLQKDNSERKAPEKIKENNSDRIVFYKHPDIFDKLRKAYNYKYMLVSRETTTENSGLFDVTIYPRGERDLAKTRKLIPKSNGLDPKIYGGYSGNTDAYMVIVKIDKGKESIYKVIGVPMRALASLNRAKKQGNYKEELHQVLEPQIMFDKNGKPKRSVKGFRIIKDHVPFKQVVLDGDKKFMLNSSTYEINAKQLTLTPETMRIVTDNLKKGEDQDQLLVKAYDEILQKVDQYLPLFDVNKFRNSLHLGRAKFLDLAVNDKKITLTNILNGLHDNLVTPDLKNIGIKTPLGKLQVPSGIVLSSEAILIFQSPTGLFEKRVRIADLKRPAATKKAGQAKKKKGSYPYDVPDYAYPYDVPDYAYPYDVPDYA

SEQ ID NO: 42 - protein, 1373 aa, synthetic construct
MPAAKRVKLDGSEVEFSHEYWMRHALTLAKRARDEREVPVGAVLVLNNRVIGEGWNRAIGLHDPTAHAEIMALRQGGLVMQNYRLYDATLYVTFEPCVMCAGAMIHSRIGRVVFGVRNAKTGAAGSLMDVLHHPGMNHRVEITEGILADECAALLCRFFRMPRRVFNAQKKAQSSTDGSSGSETPGTSESATPESSGPKKKRKVGTMSNGKILGLAIGIASVGVGVIDAQTGEIIHASSRIFPSANAANNAERRTFRGSRRLIRRKKHRIKRLDDLFNDFHINLDGEMSTDNPYVLRVKGLSQKLTVEELYISIKNIMKRRGISYLDDAESDNEAGRSDYAKAIERNRQLLTSKTPGEIQLERLEKYGQLRGNFTIIDEEGQSQQIINVFSTSDYVKEVEKILDCQKMYHKFISDEFCDKLIELLREKRKYYVGPGNEKSRTDYGIYRTDGTTLENLFGILIGKCTFYPDQYRSSRASYTAQEFNFLNDLNNLTVPTETKKLSQEQKEFLVNYAKETSVLGAGKILQQIAKLADCKVEDIRGYRLDNKDKPELHTFETYRAMKGLVPLVDIGVLSREQLDILADILTLNTDFEGIREALKKQLPNVFDEKQVKGLASFRKSKSQLFAKGWHNLSQKIMLEVIPELYATSDEQMTILTRLGKFEKSSVAEYPSSINVDEITDEIYNPVVAKSIRQTIKIINASIKKWDEFDQIVIEMPRDRNEDEEKKRIADGQKANAKEKADSILRAAELYCAGKVLPDYVYNGHNQLATKIRLWYQQGERCIYTGQPISIHDLIHNQNQYEIDHILPLSLTFDDSLSNKVLVLATANQEKAQRTPYNYLKSATSAWSYREFKDYVTKRKGIGKKKCEYLTFEEDINGFEVRSKFIQRNLVDTRYASKVILNALQDYFKISGIQTKVSVVRGQFTSQLRHKWGIEKTRETYHHHAVDALIIAASSQLRLWKKQESPLVVDYQEGRQVDLETGEILELTDEQYKELVYQPPYQGFVNTISSSAFDNEILFSYQVDSKVNRKISDATIYATRNAQLGKDKTEGIYVLGKIKDIYTQAGYEAFLKRYTKDKTSFLMYHKDLDTWEKVIEIILRDYREYDEKGKEIGNPFERYRRENGYVKKYSRKGNGTAIKSLKYYDNKLGNHIDITPENSRNAVVLQSLKPWRTDVYFNKETGKYEFLGIKYSDLSFEKGTGEYGISQEKYDSIKIAEGVAKKSIFKFTLYKQDLLFIKDIENNFGKLLRFTSKNDTSKHYVELKPYDKNKFGTEEPLLPVLGNVAKSGQCIKGLNKSNISIYKVRTDILGYRHFIKQEGEHPQLKFKKKRPAATKKAGQAKKKKGSYPYDVPDYAYPYDVPDYAYPYDVPDYA

SEQ ID NO: 43 - RNA, 97 nt, synthetic construct
  source 1..97: mol_type = other RNA
gttttagtactctgtaattttgaaaaaaattacagaatctactaaaacaaggcaaaatgccgtgtttatctcgtcaacttgttgacgagattttttt

SEQ ID NO: 44 - RNA, 93 nt, synthetic construct
  source 1..93: mol_type = other RNA
gttttagagctatgttggaaacaacatagcgagttaaaataaggcattgtccgttatcagcttttaaagcaagcactgtctcggtgctttttt

SEQ ID NO: 45 - RNA, 98 nt, synthetic construct
  source 1..98: mol_type = other RNA
gtttttgtactctcaagatttgaaaaaatcttgcagagcctacaaagataaggcttcatgccgaattcaagcaccccatgtttacatggggtgctttt

SEQ ID NO: 46 - RNA, 99 nt, synthetic construct
  source 1..99: mol_type = other RNA
gttttagtactctgtaattttgaaaaaaattacagaatctactaaaacaaggcaaaatgccgtgtttatctcgtcaacttgtttggcgagatttttttt

SEQ ID NO: 47 - RNA, 95 nt, synthetic construct
  source 1..95: mol_type = other RNA
gtttttgtactctcaagatttgaaaaaatcttgcagaagctacaaagataaggcttcatgccgaaatcaacaccctgtctatgacggggtgtttt

SEQ ID NO: 48 - RNA, 96 nt, synthetic construct
  source 1..96: mol_type = other RNA
gttttagagctgtgttgtgaaaacaacacagcaagttaaaataaggctttgtccgtacacaacttgaaaaagtgcgcaccgattcggtgctttttt

SEQ ID NO: 49 - RNA, 99 nt, synthetic construct
  source 1..99: mol_type = other RNA
gtttttgtactctcaagatttgaaaaaatcttgcagaagctacaaagataaggcttcatgccgaaatcaacaccctgtcattttatggcagggtgtttt

SEQ ID NO: 50 - RNA, 98 nt, synthetic construct
  source 1..98: mol_type = other RNA
gtttttgtactctcaagatttgaaaaaatcttgcagaagctacaaagataaggcttcatgccgaattcaacaccctgtcatttatggcggggtgtttt

SEQ ID NO: 51 - RNA, 103 nt, synthetic construct
  source 1..103: mol_type = other RNA
gtttttgtactctcaagatttgaaaaaatcttgtagaagctacaaagataaggcttcatgccgaattcaacaccctgtcatttatggcggggtgtttcgtttt

SEQ ID NO: 52 - RNA, 96 nt, synthetic construct
  source 1..96: mol_type = other RNA
gttttagtactctgtaattttgaaaaaaattacagaatctactgaaacaagactatatgtcgtgtttatcccactaatttattagtgggatttttt

SEQ ID NO: 53 - RNA, 97 nt, synthetic construct; identical to SEQ ID NO: 43
  source 1..97: mol_type = other RNA

SEQ ID NO: 54 - RNA, 100 nt, synthetic construct
  source 1..100: mol_type = other RNA
gtttttgtactctcaagatttgaaaaaatcttgctgagcctacaaagataaggctttatgccgaattcaagcaccccatgttttgacatggggtgctttt

SEQ ID NO: 55 - RNA, 127 nt, synthetic construct
  source 1..127: mol_type = other RNA
gttttagatgattgttagatcgaaagatctaacaaccagattttaaaatcaaacaatgtatctttgatactaagtttcaacgcggtattattaccgtcctgcctcagctctatagcggaggtttttt

SEQ ID NO: 56 - RNA, 97 nt, synthetic construct
  source 1..97: mol_type = other RNA
gtttttgtactctcaagatttgaaaaaatcttgcagagcctacaaagataaggctttatgccgaaatcaagcaccccgtttcgtacggggtgctttt

SEQ ID NO: 57 - protein, 208 aa, Petromyzon marinus
MTDAEYVRIHEKLDIYTFKKQFFNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWACKLYYEKNARNQIGLWNLRDNGVGLNVMVSEHYQCCRKIFIQSSHNQLNENRWLEKTLKRAEKRRSELSIMIQVKILHTTKSPAV

SEQ ID NO: 58 - protein, 145 aa, Homo sapiens
MDSLLMNRRKFLYQFKNVRWAKGRRETYLCYVVKRRDSATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKAPV

SEQ ID NO: 59 - protein, 198 aa, Homo sapiens [sequence omitted]

SEQ ID NO: 60 - protein, 198 aa, Mus sp.
MDSLLMKQKKFLYHFKNVRWAKGRHETYLCYVVKRRDSATSCSLDFGHLRNKSGCHVELLFLRYISDWDLDPGRCYRVTWFTSWSPCYDCARHVAEFLRWNPNLSLRIFTARLYFCEDRKAEPEGLRRLHRAGVQIGIMTFKDYFYCWNTFVENRERTFKAWEGLHENSVRLTRQLRRILLPLYEVDDLRDAFRMLGF

SEQ ID NO: 61 - protein, 198 aa, Canis sp.
MDSLLMKQRKFLYHFKNVRWAKGRHETYLCYVVKRRDSATSFSLDFGHLRNKSGCHVELLFLRYISDWDLDPGRCYRVTWFTSWSPCYDCARHVADFLRGYPNLSLRIFAARLYFCEDRKAEPEGLRRLHRAGVQIAIMTFKDYFYCWNTFVENREKTFKAWEGLHENSVRLSRQLRRILLPLYEVDDLRDAFRTLGL

SEQ ID NO: 62 - protein, 199 aa, Bos sp.
MDSLLKKQRQFLYQFKNVRWAKGRHETYLCYVVKRRDSPTSFSLDFGHLRNKAGCHVELLFLRYISDWDLDPGRCYRVTWFTSWSPCYDCARHVADFLRGYPNLSLRIFTARLYFCDKERKAEPEGLRRLHRAGVQIAIMTFKDYFYCWNTFVENHERTFKAWEGLHENSVRLSRQLRRILLPLYEVDDLRDAFRTLGL

SEQ ID NO: 63 - protein, 239 aa, Rattus sp.
MAVGSKPKAALVGPHWERERIWCFLCSTGLGTQQTGQTSRWLRPAATQDPVSPPRSLLMKQRKFLYHFKNVRWAKGRHETYLCYVVKRRDSATSFSLDFGYLRNKSGCHVELLFLRYISDWDLDPGRCYRVTWFTSWSPCYDCARHVADFLRGNPNLSLRIFTARLTGWGALPAGLMSPARPSDYFYCWNTFVENHERTFKAWEGLHENSVRLSRRLRRILLPLYEVDDLRDAFRTLGL

SEQ ID NO: 64 - protein, 198 aa, Canis lupus; identical to SEQ ID NO: 61

SEQ ID NO: 65 - protein, 199 aa, Bos taurus; identical to SEQ ID NO: 62

SEQ ID NO: 66 - protein, 198 aa, Mus musculus [sequence omitted]

SEQ ID NO: 67 - protein, 229 aa, Rattus norvegicus [sequence omitted]

SEQ ID NO: 68 - protein, 229 aa, Mesocricetus auratus
MSSETGPVVVDPTLRRRIEPHEFDAFFDQGELRKETCLLYEIRWGGRHNIWRHTGQNTSRHVEINFIEKFTSERYFYPSTRCSIVWFLSWSPCGECSKAITEFLSGHPNVTLFIYAARLYHHTDQRNRQGLRDLISRGVTIRIMTEQEYCYCWRNFVNYPPSNEVYWPRYPNLWMRLYALELYCIHLGLPPCLKIKRRHQYPLTFFRLNLQSCHYQRIPPHILWATGFI

SEQ ID NO: 69 - protein, 236 aa, Pongo pygmaeus
MTSEKGPSTGDPTLRRRIESWEFDVFYDPRELRKETCLLYEIKWGMSRKIWRSSGKNTTNHVEVNFIKKFTSERRFHSSISCSITWFLSWSPCWECSQAIREFLSQHPGVTLVIYVARLFWHMDQRNRQGLRDLVNSGVTIQIMRASEYYHCWRNFVNYPPGDEAHWPQYPPLWMMLYALELHCIILSLPPCLKISRRWQNHLAFFRLHLQNCHYQTIPPHILLATGLIHPSVTWR

SEQ ID NO: 70 - protein, 236 aa, Oryctolagus cuniculus
MASEKGPSNKDYTLRRRIEPWEFEVFFDPQELRKEACLLYEIKWGASSKTWRSSGKNTTNHVEVNFLEKLTSEGRLGPSTCCSITWFLSWSPCWECSMAIREFLSQHPGVTLIIFVARLFQHMDRRNRQGLKDLVTSGVTVRVMSVSEYCYCWENFVNYPPGKAAQWPRYPPRWMLMYALELYCIILGLPPCLKISRRHQKQLTFFSLTPQYCHYKMIPPYILLATGLLQPSVPWR

SEQ ID NO: 71 - protein, 235 aa, Monodelphis domestica
MNSKTGPSVGDATLRRRIKPWEFVAFFNPQELRKETCLLYEIKWGNQNIWRHSNQNTSQHAEINFMEKFTAERHFNSSVRCSITWFLSWSPCWECSKAIRKFLDHYPNVTLAIFISRLYWHMDQQHRQGLKELVHSGVTIQIMSYSEYHYCWRNFVDYPQGEEDYWPKYPYLWIMLYVLELHCIILGLPPCLKISGSHSNQLALFSLDLQDCHYQKIPYNVLVATGLVQPFVTWR

SEQ ID NO: 72 - protein, 224 aa, Pongo pygmaeus
MAQKEEAAAATEAASQNGEDLENLDDPEKLKELIELPPFEIVTGERLPANFFKFQFRNVEYSSGRNKTFLCYVVEAQGKGGQVQASRGYLEDEHAAAHAEEAFFNTILPAFDPALRYNVTWYVSSSPCAACADRIIKTLSKTKNLRLLILVGRLFMWEELEIQDALKKLKEAGCKLRIMKPQDFEYVWQNFVEQEEGESKAFQPWEDIQENFLYYEEKLADILK

SEQ ID NO: 73 - protein, 224 aa, Bos taurus
MAQKEEAAAAAEPASQNGEEVENLEDPEKLKELIELPPFEIVTGERLPAHYFKFQFRNVEYSSGRNKTFLCYVVEAQSKGGQVQASRGYLEDEHATNHAEEAFFNSIMPTFDPALRYMVTWYVSSSPCAACADRIVKTLNKTKNLRLLILVGRLFMWEEPEIQAALRKLKEAGCRLRIMKPQDFEYIWQNFVEQEEGESKAFEPWEDIQENFLYYEEKLADILK

SEQ ID NO: 74 - protein, 440 aa, Mus musculus
MQPQRLGPRAGMGPFCLGCSHRKCYSPIRNLISQETFKFHFKNLGYAKGRKDTFLCYEVTRKDCDSPVSLHHGVFKNKDNIHAEICFLYWFHDKVLKVLSPREEFKITWYMSWSPCFECAEQIVRFLATHHNLSLDIFSSRLYNVQDPETQQNLCRLVQEGAQVAAMDLYEFKKCWKKFVDNGGRRFRPWKRLLTNFRYQDSKLQEILRPCYISVPSSSSSTLSNICLTKGLPETRFWVEGRRMDPLSEEEFYSQFYNQRVKHLCYYHRMKPYLCYQLEQFNGQAPLKGCLLSEKGKQHAEILFLDKIRSMELSQVTITCYLTWSPCPNCAWQLAAFKRDRPDLILHIYTSRLYFHWKRPFQKGLCSLWQSGILVDVMDLPQFTDCWTNFVNPKRPFWPWKGLEIISRRTQRRLRRIKESWGLQDLVNDFGNLQLGPPMS

SEQ ID NO: 75 - protein, 429 aa, Mus sp.
MGPFCLGCSHRKCYSPIRNLISQETFKFHFKNLGYAKGRKDTFLCYEVTRKDCDSPVSLHHGVFKNKDNIHAEICFLYWFHDKVLKVLSPREEFKITWYMSWSPCFECAEQIVRFLATHHNLSLDIFSSRLYNVQDPETQQNLCRLVQEGAQVAAMDLYEFKKCWKKFVDNGGRRFRPWKRLLTNFRYQDSKLQEILRPCYIPVPSSSSSTLSNICLTKGLPETRFCVEGRRMDPLSEEEFYSQFYNQRVKHLCYYHRMKPYLCYQLEQFNGQAPLKGCLLSEKGKQHAEILFLDKIRSMELSQVTITCYLTWSPCPNCAWQLAAFKRDRPDLILHIYTSRLYFHWKRPFQKGLCSLWQSGILVDVMDLPQFTDCWTNFVNPKRPFWPWKGLEIISRRTQRRLRRIKESWGLQDLVNDFGNLQLGPPMS

SEQ ID NO: 76 - protein, 430 aa, Rattus sp.
MGPFCLGCSHRKCYSPIRNLISQETFKFHFKNRLRYAIDRKDTFLCYEVTRKDCDSPVSLHHGVFKNKDNIHAEICFLYWFHDKVLKVLSPREEFKITWYMSWSPCFECAEQVLRFLATHHNLSLDIFSSRLYNIRDPENQQNLCRLVQEGAQVAAMDLYEFKKCWKKFVDNGGRRFRPWKKLLTNFRYQDSKLQEILRPCYIPVPSSSSSTLSNICLTKGLPETRFCVERRRVHLLSEEEFYSQFYNQRVKHLCYYHGVKPYLCYQLEQFNGQAPLKGCLLSEKGKQHAEILFLDKIRSMELSQVIITCYLTWSPCPNCAWQLAAFKRDRPDLILHIYTSRLYFHWKRPFQKGLCSLWQSGILVDVMDLPQFTDCWTNFVNPKRPFWPWKGLEIISRRTQRRLHRIKESWGLQDLVNDFGNLQLGPPMS

SEQ ID NO: 77 - protein, 199 aa, Homo sapiens
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 78 - protein, 373 aa, Homo sapiens
MKPHFRNTVERMYRDTFSYNFYNRPILSRRNTVWLCYEVKTKGPSRPRLDAKIFRGQVYSQPEHHAEMCFLSWFCGNQLPAYKCFQITWFVSWTPCPDCVAKLAEFLAEHPNVTLTISAARLYYYWERDYRRALCRLSQAGARVKIMDDEEFAYCWENFVYSEGQPFMPWYKFDDNYAFLHRTLKEILRNPMEAMYPHIFYFHFKNLRKAYGRNESWLCFTMEVVKHHSPVSWKRGVFRNQVDPETHCHAERCFLSWFCDDILSPNTNYEVTWYTSWSPCPECAGEVAEFLARHSNVNLTIFTARLYYFWDTDYQEGLRSLSQEGASVEIMGYKDFKYCWENFVYNDDEPFKPWKGLKYNFLFLDSKLQEILE

SEQ ID NO: 79 - protein, 370 aa, Macaca mulatta
MVEPMDPRTFVSNFNNRPILSGLNTVWLCCEVKTKDPSGPPLDAKIFQGKVYSKAKYHPEMRFLRWFHKWRQLHHDQEYKVTWYVSWSPCTRCANSVATFLAKDPKVTLTIFVARLYYFWKPDYQQALRILCQKRGGPHATMKIMNYNEFQDCWNKFVDGRGKPFKPRNNLPKHYTLLQATLGELLRHLMDPGTFTSNFNNKPWVSGQHETYLCYKVERLHNDTWVPLNQHRGFLRNQAPNIHGFPKGRHAELCFLDLIPFWKLDGQQYRVTCFTSWSPCFSCAQEMAKFISNNEHVSLCIFAARIYDDQGRYQEGLRALHRDGAKIAMMNYSEFEYCWDTFVDRQGRPFQPWDGLDEHSQALSGRLRAI

SEQ ID NO: 80 - protein, 384 aa, Pan sp.
MKPHFRNPVERMYQDTFSDNFYNRPILSHRNTVWLCYEVKTKGPSRPPLDAKIFRGQVYSKLKYHPEMRFFHWFSKWRKLHRDQEYEVTWYISWSPCTKCTRDVATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTSNFNNELWVRGRHETYLCYEVERLHNDTWVLLNQRRGFLCNQAPHKHGFLEGRHAELCFLDVIPFWKLDLHQDYRVTCFTSWSPCFSCAQEMAKFISNNKHVSLCIFAARIYDDQGRCQEGLRTLAKAGAKISIMTYSEFKHCWDTFVDHQGCPFQPWDGLEEHSQALSGRLRAILQNQGN

SEQ ID NO: 81 - protein, 377 aa, Chlorocebus sp.
MNPQIRNMVEQMEPDIFVYYFNNRPILSGRNTVWLCYEVKTKDPSGPPLDANIFQGKLYPEAKDHPEMKFLHWFRKWRQLHRDQEYEVTWYVSWSPCTRCANSVATFLAEDPKVTLTIFVARLYYFWKPDYQQALRILCQERGGPHATMKIMNYNEFQHCWNEFVDGQGKPFKPRKNLPKHYTLLHATLGELLRHVMDPGTFTSNFNNKPWVSGQRETYLCYKVERSHNDTWVLLNQHRGFLRNQAPDRHGFPKGRHAELCFLDLIPFWKLDDQQYRVTCFTSWSPCFSCAQKMAKFISNNKHVSLCIFAARIYDDQGRCQEGLRTLHRDGAKIAVMNYSEFEYCWDTFVDRQGRPFQPWDGLDEHSQALSGRLRAI

SEQ ID NO: 82 - protein, 384 aa, Homo sapiens
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 83 - protein, 373 aa, Homo sapiens; identical to SEQ ID NO: 78

SEQ ID NO: 84 - protein, 382 aa, Homo sapiens
MNPQIRNPMERMYRDTFYDNFENEPILYGRSYTWLCYEVKIKRGRSNLLWDTGVFRGQVYFKPQYHAEMCFLSWFCGNQLPAYKCFQITWFVSWTPCPDCVAKLAEFLSEHPNVTLTISAARLYYYWERDYRRALCRLSQAGARVTIMDYEEFAYCWENFVYNEGQQFMPWYKFDENYAFLHRTLKEILRYLMDPDTFTFNFNNDPLVLRRRQTYLCYEVERLDNGTWVLMDQHMGFLCNEAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFEYCWDTFVYRQGCPFQPWDGLEEHSQALSGRLRAILQNQGN

SEQ ID NO: 85 - protein, 395 aa, Rattus sp.
MQPQGLGPNAGMGPVCLGCSHRRPYSPIRNPLKKLYQQTFYFHFKNVRYAWGRKNNFLCYEVNGMDCALPVPLRQGVFRKQGHIHAELCFIYWFHDKVLRVLSPMEEFKVTWYMSWSPCSKCAEQVARFLAAHRNLSLAIFSSRLYYYLRNPNYQQKLCRLIQEGVHVAAMDLPEFKKCWNKFVDNDGQPFRPWMRLRINFSFYDCKLQEIFSRMNLLREDVFYLQFNNSHRVKPVQNRYYRRKSYLCYQLERANGQEPLKGYLLYKKGEQHVEILFLEKMRSMELSQVRITCYLTWSPCPNCARQLAAFKKDHPDLILRIYTSRLYFWRKKFQKGLCTLWRSGIHVDVMDLPQFADCWTNFVNPQRPFRPWNELEKNSWRIQRRLRRIKESWGL

SEQ ID NO: 86 - protein, 227 aa, Bos sp.
MDGWEVAFRSGTVLKAGVLGVSMTEGWAGSGHPGQGACVWTPGTRNTMNLLREVLFKQQFGNQPRVPAPYYRRKTYLCYQLKQRNDLTLDRGCFRNKKQRHAERFIDKINSLDLNPSQSYKIICYITWSPCPNCANELVNFITRNNHLKLEIFASRLYFHWIKSFKMGLQDLQNAGISVAVMTHTEFEDCWEQFVDNQSRPFQPWDKLEQYSASIRRRLQRILTAPI

SEQ ID NO: 87 - protein, 490 aa, Pan sp.
MNPQIRNPMEWMYQRTFYYNFENEPILYGRSYTWLCYEVKIRRGHSNLLWDTGVFRGQMYSQPEHHAEMCFLSWFCGNQLSAYKCFQITWFVSWTPCPDCVAKLAKFLAEHPNVTLTISAARLYYYWERDYRRALCRLSQAGARVKIMDDEEFAYCWENFVYNEGQPFMPWYKFDDNYAFLHRTLKEIIRHLMDPDTFTFNFNNDPLVLRRHQTYLCYEVERLDNGTWVLMDQHMGFLCNEAKNLLCGFYGRHAELRFLDLVPSLQLDPAQIYRVTWFISWSPCFSWGCAGQVRAFLQENTHVRLRIFAARIYDYDPLYKEALQMLRDAGAQVSIMTYDEFEYCWDTFVYRQGCPFQPWDGLEEHSQALSGRLRAILQVRASSLCMVPHRPPPPPQSPGPCLPLCSEPPLGSLLPTGRPAPSLPFLLTASFSFPPPASLPPLPSLSLSPGHLPVPSFHSLTSCSIQPPCSSRIRETEGWASVSKEGRDLG

SEQ ID NO: 88 - protein, 190 aa, Homo sapiens
MNPQIRNPMKAMYPGTFYFQFKNLWEANDRNETWLCFTVEGIKRRSVVSWKTGVFRNQVDSETHCHAERCFLSWFCDDILSPNTKYQVTWYTSWSPCPDCAGEVAEFLARHSNVNLTIFTARLYYFQYPCYQEGLRSLSQEGVAVEIMDYEDFKYCWENFVYNDNEPFKPWKGLKTNFRLLKRRLRESLQ

SEQ ID NO: 89 - protein, 190 aa, Gorilla sp.
MNPQIRNPMKAMYPGTFYFQFKNLWEANDRNETWLCFTVEGIKRRSVVSWKTGVFRNQVDSETHCHAERCFLSWECDDILSPNTNYQVTWYTSWSPCPECAGEVAEFLARHSNVNLTIFTARLYYFQDTDYQEGLRSLSQEGVAVKIMDYKDFKYCWENFVYNDDEPFKPWKGLKYNFRFLKRRLQEILE

SEQ ID NO: 90 - protein, 199 aa, Homo sapiens; identical to SEQ ID NO: 77

SEQ ID NO: 91 - protein, 202 aa, Macaca mulatta
MDGSPASRPRHLMDPNTFTFNFNNDLSVRGRHQTYLCYEVERLDNGTWVPMDERRGFLCNKAKNVPCGDYGCHVELRFLCEVPSWQLDPAQTYRVTWFISWSPCFRRGCAGQVRVFLQENKHVRLRIFAARIYDYDPLYQEALRTLRDAGAQVSIMTYEEFKHCWDTFVDRQGRPFQPWDGLDEHSQALSGRLRAILQNQGN

SEQ ID NO: 92 - protein, 185 aa, Bos sp.
MDEYTFTENFNNQGWPSKTYLCYEMERLDGDATIPLDEYKGFVRNKGLDQPEKPCHAELYFLGKIHSWNLDRNQHYRLTCFISWSPCYDCAQKLTTFLKENHHISLHILASRIYTHNRFGCHQSGLCELQAAGARITIMTFEDFKHCWETFVDHKGKPFQPWEGLNVKSQALCTELQAILKTQQN

SEQ ID NO: 93 - protein, 200 aa, Homo sapiens
MALLTAETFRLQFNNKRRLRRPYYPRKALLCYQLTPQNGSTPTRGYFENKKKCHAEICFINEIKSMGLDETQCYQVTCYLTWSPCSSCAWELVDFIKAHDHLNLGIFASRLYYHWCKPQQKGLRLLCGSQVPVEVMGFPKFADCWENFVDHEKPLSFNPYKMLEELDKNSRAIKRRLERIKIPGVRAQGRYMDILCDAEV

SEQ ID NO: 94 - protein, 210 aa, Macaca mulatta
MALLTAKTFSLQFNNKRRVNKPYYPRKALLCYQLTPQNGSTPTRGHLKNKKKDHAEIRFINKIKSMGLDETQCYQVTCYLTWSPCPSCAGELVDFIKAHRHLNLRIFASRLYYHWRPNYQEGLLLLCGSQVPVEVMGLPEFTDCWENFVDHKEPPSFNPSEKLEELDKNSQAIKRRLERIKSRSVDVLENGLRSLQLGPVTPSSSIRNSR

SEQ ID NO: 95 - protein, 386 aa, Homo sapiens
MNPQIRNPMERMYRDTFYDNFENEPILYGRSYTWLCYEVKIKRGRSNLLWDTGVFRGPVLPKRQSNHRQEVYFRFENHAEMCFLSWFCGNRLPANRRFQITWFVSWNPCLPCVVKVTKFLAEHPNVTLTISAARLYYYRDRDWRWVLLRLHKAGARVKIMDYEDFAYCWENFVCNEGQPFMPWYKFDDNYASLHRTLKEILRNPMEAMYPHIFYFHFKNLLKACGRNESWLCFTMEVTKHHSAVFRKRGVFRNQVDPETHCHAERCFLSWFCDDILSPNTNYEVTWYTSWSPCPECAGEVAEFLARHSNVNLTIFTARLCYFWDTDYQEGLCSLSQEGASVKIMGYKDFVSCWKNFVYSDDEPFKPWKGLQTNFRLLKRRLREILQ

SEQ ID NO: 96 - protein, 236 aa, Homo sapiens
MTSEKGPSTGDPTLRRRIEPWEFDVFYDPRELRKEACLLYEIKWGMSRKIWRSSGKNTTNHVEVNFIKKFTSERDFHPSMSCSITWFLSWSPCWECSQAIREFLSRHPGVTLVIYVARLFWHMDQQNRQGLRDLVNSGVTIQIMRASEYYHCWRNFVNYPPGDEAHWPQYPPLWMMLYALELHCIILSLPPCLKISRRWQNHLTFFRLHLQNCHYQTIPPHILLATGLIHPSVAWR

SEQ ID NO: 97 - protein, 229 aa, Mus sp.
MSSETGPVAVDPTLRRRIEPHEFEVFFDPRELRKETCLLYEINWGGRHSVWRHTSQNTSNHVEVNFLEKFTTERYFRPNTRCSITWFLSWSPCGECSRAITEFLSRHPYVTLFIYIARLYHHTDQRNRQGLRDLISSGVTIQIMTEQEYCYCWRNFVNYPPSNEAYWPRYPHLWVKLYVLELYCIILGLPPCLKILRRKQPQLTFFTITLQTCHYQRIPPHLLWATGLK

SEQ ID NO: 98 - protein, 229 aa, Rattus sp. [sequence omitted]

SEQ ID NO: 99 - protein, 224 aa, Homo sapiens
MAQKEEAAVATEAASQNGEDLENLDDPEKLKELIELPPFEIVTGERLPANFFKFQFRNVEYSSGRNKTFLCYVVEAQGKGGQVQASRGYLEDEHAAAHAEEAFFNTILPAFDPALRYNVTWYVSSSPCAACADRIIKTLSKTKNLRLLILVGRLFMWEEPEIQAALKKLKEAGCKLRIMKPQDFEYVWQNFVEQEEGESKAFQPWEDIQENFLYYEEKLADILK

SEQ ID NO: 100 - protein, 224 aa, Mus sp.
MAQKEEAAEAAAPASQNGDDLENLEDPEKLKELIDLPPFEIVTGVRLPVNFFKFQFRNVEYSSGRNKTFLCYVVEVQSKGGQAQATQGYLEDEHAGAHAEEAFFNTILPAFDPALKYNVTWYVSSSPCAACADRILKTLSKTKNLRLLILVSRLFMWEEPEVQAALKKLKEAGCKLRIMKPQDFEYIWQNFVEQEEGESKAFEPWEDIQENFLYYEEKLADILK

SEQ ID NO: 101 - protein, 224 aa, Rattus sp.
MAQKEEAAEAAAPASQNGDDLENLEDPEKLKELIDLPPFEIVTGVRLPVNFFKFQFRNVEYSSGRNKTFLCYVVEAQSKGGQVQATQGYLEDEHAGAHAEEAFFNTILPAFDPALKYNVTWYVSSSPCAACADRILKTLSKTKNLRLLILVSRLFMWEEPEVQAALKKLKEAGCKLRIMKPQDFEYLWQNFVEQEEGESKAFEPWEDIQENFLYYEEKLADILK

SEQ ID NO: 102 - protein, 224 aa, Bos sp.; identical to SEQ ID NO: 73

SEQ ID NO: 103 - protein, 208 aa, Petromyzon marinus
MTDAEYVRIHEKLDIYTFKKQFFNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWACKLYYEKNARNQIGLWNLRDNGVGLNVMVSEHYQCCRKIFIQSSHNQLNENRWLEKTLKRAEKRRSELSFMIQVKILHTTKSPAV

SEQ ID NO: 104 - protein, 381 aa, Homo sapiens
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKFNYDEFQHCWSKFVYSQRELFEPWNNLPKYYILLHFMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKKHVSLCIFTARIYRRQGRCQEGLRTLAEAGAKISFTYSEFKHCWDTFVDHQGCPFQPWDGLDEHSQDLSGRLRAILQNQEN

SEQ ID NO: 105 - protein, 182 aa, Homo sapiens
MDPPTFTFNFNNEPWWGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISFTYSEFKHCWDTFVDHQGCPFQPWDGLDEHSQDLSGRLRAILQ

SEQ ID NO: 106 - protein, 184 aa, Homo sapiens
MDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYRRQGRCQEGLRTLAEAGAKISFMTYSEFKHCWDTFVDHQGCPFQPWDGLDEHSQDLSGRLRAILQ

SEQ ID NO: 107 - protein, 367 aa, Homo sapiens
MEPIYEEYLANHGTIVKPYYWLSFSLDCSNCPYHIRTGEEARVSLTEFCQIFGFPYGTTFPQTKHLTFYELKTSSGSLVQKGHASSCTGNYIHPESMLFEMNGYLDSAIYNNDSIRHIILYSNNSPCNEANHCCISKMYNFLITYPGITLSIYFSQLYHTEMDFPASAWNREALRSLASLWPRVVLSPISGGIWHSVLHSFISGVSGSHVFQPILTGRALADRHNAYEINAITGVKPYFTDVLLQTKRNPNTKAQEALESYPLNNAFPGQFFQMPSGQLQPNLPPDLRAPVVFVLVPLRDLPPMHMGQNPNKPRNIVRHLNMPQMSFQETKDLGRLPTGRSVEIVEITEQFASSKEADEKKKKKGKK

SEQ ID NO: 108 - protein, 198 aa, Mus musculus; identical to SEQ ID NO: 60

SEQ ID NO: 109 - protein, 388 aa, Rattus norvegicus
MEPLYEEYLTHSGTIVKPYYWLSVSLNCTNCPYHIRTGEEARVPYTEFHQTFGFPWSTYPQTKHLTFYELRSSSGNLIQKGLASNCTGSHTHPESMLFERDGYLDSLIFHDSNIRHIILYSNNSPCDEANHCCISKMYNFLMNYPEVTLSVFFSQLYHTENQFPTSAWNREALRGLASLWPQVTLSAISGGIWQSILETFVSGISEGLTAVRPFTAGRTLTDRYNAYEINCITEVKPYFTDALHSWQKENQDQKVWAASENQPLHNTTPAQWQPDMSQDCRTPAVFMLVPYRDLPPIHVNPSPQKPRTVVRHLNTLQLSASKVKALRKSPSGRPVKKEEARKGSTRSQEANETNKSKWKKQTLFIKSNICHLLEREQKKIGILSSWSV

SEQ ID NO: 110 - protein, 363 aa, Macaca fascicularis
MEPTYEEYLANHGTIVKPYYWLSFSLDCSNCPYHIRTGEEARVSLTEFCQIFGFPYGTTYPQTKHLTFYELKTSSGSLVQKGHASSCTGNYIHPESMLFEMNGYLDSAIYNNDSIRHIILYCNNSPCNEANHCCISKVYNFLITYPGITLSIYFSQLYHTEMDFPASAWNREALRSLASLWPRVVLSPISGGIWHSVLHSFVSGVSGSHVFQPILTGRALTDRYNAYEINAITGVKPFFTDVLLHTKRNPNTKAQMALESYPLNNAFPGQSFQMTSGIPPDLRAPVVFVLLPLRDLPPMHMGQDPNKPRNIIRHLNMPQMSFQETKDLERLPTRRSVETVEITERFASSKQAEEKTKKKKGKK

SEQ ID NO: 111 - protein, 224 aa, Petromyzon marinus
MAGYECVRVSEKLDFDTFEFQFENLHYATERHRTYVIFDVKPQSAGGRSRRLWGYIINNPNVCHAELILMSMIDRHLESNPGVYAMTWYMSWSPCANCSSKLNPWLKNLLEEQGHTLTMHFSRIYDRDREGDHRGLRGLKHVSNSFRMGVVGRAEVKECLAEYVEASRRTLTWLDTTESMAAKMRRKLFCILVRCAGMRESGIPLHLFTLQTPLLSGRVVWWRV

SEQ ID NO: 112 - protein, 331 aa, Petromyzon marinus
MELREVVDCALASCVRHEPLSRVAFLRCFAAPSQKPRGTVILFYVEGAGRGVTGGHAVNYNKQGTSIHAEVLLLSAVRAALLRRRRCEDGEEATRGCTLHCYSTYSPCRDCVEYIQEFGASTGVRVVIHCCRLYELDVNRRRSEAEGVLRSLSRLGRDFRLMGPRDAIALLLGGRLANTADGESGASGNAWVTETNVVEPLVDMTGFGDEDLHAQVQRNKQIREAYANYASAVSLMLGELHVDPDKFPFLAEFLAQTSVEPSGTPRETRGRPRGASSRGPEIGRQRPADFERALGAYGLFLHPRIVSREADREEIKRDLIVVMRKHNYQGP

SEQ ID NO: 113 - protein, 209 aa, Petromyzon marinus
MAGDENVRVSEKLDFDTFEFQFENLHYATERHRTYVIFDVKPQSAGGRSRRLWGYIINNPNVCHAELILMSMIDRHLESNPGVYAMTWYMSWSPCANCSSKLNPWLKNLLEEQGHTLMMHFSRIYDRDREGDHRGLRGLKHVSNSFRMGVVGRAEVKECLAEYVEASRRTLTWLDTTESMAAKMRRKLFCILVRCAGMRESGMPLHLFT

SEQ ID NO: 114 - protein, 158 aa, Saccharomyces cerevisiae
MVTGGMASKWDQKGMDIAYEEAALGYKEGGVPIGGCLINNKDGSVLGRGHNMRFQKGSATLHGEISTLENCGRLEGKVYKDTTLYTTLSPCDMCTGAIIMYGIPRCVVGENVNFKSKGEKYLQTRGHEVVVVDDERCKKIMKQFIDERPQDWFEDIGE

SEQ ID NO: 115 - protein, 218 aa, synthetic construct
MSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRGLPPCLNILRRKQPQLTFFTIALQSCHYQRLPPHILWATGLK

SEQ ID NO: 116 - protein, 218 aa, synthetic construct
MSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQHYQRLPPHILWATGLK

SEQ ID NO: 117 - protein, 429 aa, Mus sp.; identical to SEQ ID NO: 75

SEQ ID NO: 118 - protein, 167 aa, synthetic construct
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 119 - DNA, 20 bp, synthetic construct
  source 1..20: mol_type = other DNA
ggtcgtcaatgaaaggagat

SEQ ID NO: 120 - DNA, 20 bp, synthetic construct
  source 1..20: mol_type = other DNA
gctaggaatattgaaggggg

SEQ ID NO: 121 - DNA, 20 bp, synthetic construct
  source 1..20: mol_type = other DNA
gctcccatcacatcaaccgg

SEQ ID NO: 122 - DNA, 20 bp, synthetic construct
  source 1..20: mol_type = other DNA
gggcaaccacaaacccacga

SEQ ID NO: 123 - DNA, 20 bp, synthetic construct
  source 1..20: mol_type = other DNA
gagtcgacgagttgaagatg

SEQ ID NO: 124 - DNA, 20 bp, synthetic construct
  source 1..20: mol_type = other DNA
gtctgttactcgcctgtcaa

SEQ ID NO: 125 - DNA, 20 bp, synthetic construct
  source 1..20: mol_type = other DNA
ataaaaggaaaagtcactct

SEQ ID NO: 126 - DNA, 20 bp, synthetic construct
  source 1..20: mol_type = other DNA
gtgccagaaacaggggtgac

SEQ ID NO: 127 - DNA, 20 bp, synthetic construct
  source 1..20: mol_type = other DNA
gtgggcaaccacaaacccac

SEQ ID NO: 128 - DNA, 20 bp, synthetic construct
  source 1..20: mol_type = other DNA
gcagagcaaataccagagat

SEQ ID NO: 129 - DNA, 20 bp, synthetic construct
  source 1..20: mol_type = other DNA
ggtgctcaatgaaaggagat

SEQ ID NO: 130 - DNA, 20 bp, synthetic construct
  source 1..20: mol_type = other DNA
cgagcagcgtcttcgagagt

SEQ ID NO: 131 - DNA, 20 bp, synthetic construct
  source 1..20: mol_type = other DNA
gctcccatcacatcaaccgg

SEQ ID NO: 132 - DNA, 20 bp, synthetic construct
  source 1..20: mol_type = other DNA
gggcaaccacaaacccacga

SEQ ID NO: 133 - DNA, 20 bp, synthetic construct
  source 1..20: mol_type = other DNA
gagtcgacgagttgaagatg

SEQ ID NO: 134 - DNA, 20 bp, synthetic construct
  source 1..20: mol_type = other DNA
tggccatcaaggatgcccac

SEQ ID NO: 135 - DNA, 20 bp, synthetic construct
  source 1..20: mol_type = other DNA
cccccaccaaggttcacagc

SEQ ID NO: 136 - DNA, 20 bp, synthetic construct
  source 1..20: mol_type = other DNA
agaatggcagtgcaatacgt

SEQ ID NO: 137 - DNA, 20 bp, synthetic construct
  source 1..20: mol_type = other DNA
gtagggctagaggggtgagg

SEQ ID NO: 138 - DNA, 20 bp, synthetic construct
  source 1..20: mol_type = other DNA
gaacacaaagcatagactgc

SEQ ID NO: 139 - DNA, 20 bp, synthetic construct
  source 1..20: mol_type = other DNA
ggtggcactgcggctggagg

SEQ ID NO: 140 - DNA, 21 bp, synthetic construct
  source 1..21: mol_type = other DNA
gaggtcagaaatagggggtcc

SEQ ID NO: 141 - DNA, 21 bp, synthetic construct
  source 1..21: mol_type = other DNA
gtagggctagaggggtgaggc

SEQ ID NO: 142 - DNA, 21 bp, synthetic construct
  source 1..21: mol_type = other DNA
gttaagaacacgttttaaagg

SEQ ID NO: 143 - DNA, 20 bp, synthetic construct
  source 1..20: mol_type = other DNA
gaacacaaagcatagactgc

SEQ ID NO: 144 - DNA, 20 bp, synthetic construct
  source 1..20: mol_type = other DNA
ggtggcactgcggctggagg

SEQ ID NO: 145 - DNA, 21 bp, synthetic construct
  source 1..21: mol_type = other DNA
gaggtcagaaatagggggtcc

SEQ ID NO: 146 - DNA, 21 bp, synthetic construct
  source 1..21: mol_type = other DNA
gtagggctagaggggtgaggc

SEQ ID NO: 147 - DNA, 20 bp, synthetic construct
  source 1..20: mol_type = other DNA
gttaagaacacgtttaaagg

SEQ ID NO: 148 - DNA, 20 bp, synthetic construct
  source 1..20: mol_type = other DNA
gaacacaaagcatagactgc

SEQ ID NO: 149 - DNA, 20 bp, synthetic construct
  source 1..20: mol_type = other DNA
ggtggcactgcggctggagg

SEQ ID NO: 150 - DNA, 21 bp, synthetic construct
  source 1..21: mol_type = other DNA
gaggtcagaaatagggggtcc

SEQ ID NO: 151 - DNA, 21 bp, synthetic construct
  source 1..21: mol_type = other DNA
gtagggctagaggggtgaggc

SEQ ID NO: 152 - DNA, 20 bp, synthetic construct
  source 1..20: mol_type = other DNA
gttaagaacacgtttaaagg

SEQ ID NO: 153 - DNA, 20 bp, synthetic construct
  source 1..20: mol_type = other DNA
gtctgttactcgcctgtcaa

SEQ ID NO: 154 - DNA, 20 bp, synthetic construct
  source 1..20: mol_type = other DNA
gtgggcaaccacaaacccac

SEQ ID NO: 155 - DNA, 20 bp, synthetic construct
  source 1..20: mol_type = other DNA
gcagagcaaataccagagat

SEQ ID NO: 156 - DNA, 20 bp, synthetic construct
  source 1..20: mol_type = other DNA
ggtgctcaatgaaaggagat

SEQ ID NO: 157 - DNA, 20 bp, synthetic construct
  source 1..20: mol_type = other DNA
cgagcagcgtcttcgagagt

SEQ ID NO: 158 - DNA, 20 bp, synthetic construct
  source 1..20: mol_type = other DNA
gctcccatcacatcaaccgg

SEQ ID NO: 159 - DNA, 20 bp, synthetic construct
  source 1..20: mol_type = other DNA
gggcaaccacaaacccacga

SEQ ID NO: 160 - DNA, 20 bp, synthetic construct
  source 1..20: mol_type = other DNA
gagtcgacgagttgaagatg

SEQ ID NO: 161 - DNA, 20 bp, synthetic construct
  source 1..20: mol_type = other DNA
gaacacaaagcatagactgc

SEQ ID NO: 162 - DNA, 20 bp, synthetic construct
  source 1..20: mol_type = other DNA
ggtggcactgcggctggagg

SEQ ID NO: 163 - DNA, 21 bp, synthetic construct
  source 1..21: mol_type = other DNA
gaggtcagaaatagggggtcc

SEQ ID NO: 164 - DNA, 21 bp, synthetic construct
  source 1..21: mol_type = other DNA
gtagggctagaggggtgaggc

SEQ ID NO: 165 - DNA, 21 bp, synthetic construct
  source 1..21: mol_type = other DNA
gttaagaacacgttttaaagg

SEQ ID NO: 166 - DNA, 20 bp, synthetic construct
  source 1..20: mol_type = other DNA
gaacacaaagcatagactgc

SEQ ID NO: 167 - DNA, 20 bp, synthetic construct
  source 1..20: mol_type = other DNA
ggtggcactgcggctggagg

SEQ ID NO: 168 - DNA, 21 bp, synthetic construct
  source 1..21: mol_type = other DNA
gaggtcagaaatagggggtcc

SEQ ID NO: 169 - DNA, 21 bp, synthetic construct
  source 1..21: mol_type = other DNA
gtagggctagaggggtgaggc

SEQ ID NO: 170 - DNA, 20 bp, synthetic construct
  source 1..20: mol_type = other DNA
gttaagaacacgtttaaagg

SEQ ID NO: 171 - DNA, 20 bp, synthetic construct
  source 1..20: mol_type = other DNA
gaacacaaagcatagactgc

SEQ ID NO: 172 - DNA, 20 bp, synthetic construct
  source 1..20: mol_type = other DNA
ggtggcactgcggctggagg

SEQ ID NO: 173 - DNA, 21 bp, synthetic construct
  source 1..21: mol_type = other DNA
gaggtcagaaatagggggtcc

SEQ ID NO: 174 - DNA, 21 bp, synthetic construct
  source 1..21: mol_type = other DNA
gtagggctagaggggtgaggc

SEQ ID NO: 175 - DNA, 20 bp, synthetic construct
  source 1..20: mol_type = other DNA
gttaagaacacgtttaaagg

SEQ ID NO: 176 - DNA, 20 bp, synthetic construct
  source 1..20: mol_type = other DNA
gaacacaaagcatagactgc

SEQ ID NO: 177 - DNA, 20 bp, synthetic construct
  source 1..20: mol_type = other DNA
ggtggcactgcggctggagg

SEQ ID NO: 178 - DNA, 21 bp, synthetic construct
  source 1..21: mol_type = other DNA
gaggtcagaaatagggggtcc

SEQ ID NO: 179 - DNA, 21 bp, synthetic construct
  source 1..21: mol_type = other DNA
gtagggctagaggggtgaggc

SEQ ID NO: 180 - DNA, 20 bp, synthetic construct
  source 1..20: mol_type = other DNA
gttaagaacacgtttaaagg

SEQ ID NO: 181 - DNA, 20 bp, synthetic construct
  source 1..20: mol_type = other DNA
gaacacaaagcatagactgc

SEQ ID NO: 182 - DNA, 20 bp, synthetic construct
  source 1..20: mol_type = other DNA
ggtggcactgcggctggagg

SEQ ID NO: 183 - DNA, 21 bp, synthetic construct
  source 1..21: mol_type = other DNA
gaggtcagaaatagggggtcc

SEQ ID NO: 184 - DNA, 21 bp, synthetic construct
  source 1..21: mol_type = other DNA
gtagggctagaggggtgaggc

SEQ ID NO: 185 - DNA, 20 bp, synthetic construct
  source 1..20: mol_type = other DNA
gttaagaacacgtttaaagg

SEQ ID NO: 186 - DNA, 20 bp, synthetic construct
  source 1..20: mol_type = other DNA
gaacacaaagcatagactgc

SEQ ID NO: 187 - DNA, 20 bp, synthetic construct
  source 1..20: mol_type = other DNA
ggtggcactgcggctggagg

SEQ ID NO: 188 - DNA, 21 bp, synthetic construct
  source 1..21: mol_type = other DNA
gaggtcagaaatagggggtcc

SEQ ID NO: 189 - DNA, 21 bp, synthetic construct
  source 1..21: mol_type = other DNA
gtagggctagaggggtgaggc

SEQ ID NO: 190 - DNA, 20 bp, synthetic construct
  source 1..20: mol_type = other DNA
gttaagaacacgtttaaagg

SEQ ID NO: 191 - DNA, 20 bp, synthetic construct
  source 1..20: mol_type = other DNA
gaacacaaagcatagactgc

SEQ ID NO: 192 - DNA, 20 bp, synthetic construct
  source 1..20: mol_type = other DNA
ggtggcactgcggctggagg

SEQ ID NO: 193 - DNA, 21 bp, synthetic construct
  source 1..21: mol_type = other DNA
gaggtcagaaatagggggtcc

SEQ ID NO: 194 - DNA, 21 bp, synthetic construct
  source 1..21: mol_type = other DNA
gtagggctagaggggtgaggc

SEQ ID NO: 195 - DNA, 20 bp, synthetic construct
  source 1..20: mol_type = other DNA
gttaagaacacgtttaaagg

SEQ ID NO: 196 - DNA, 20 bp, synthetic construct
  source 1..20: mol_type = other DNA
gctaaagagggaatgggctt

SEQ ID NO: 197 - DNA, 20 bp, synthetic construct
  source 1..20: mol_type = other DNA
agacacacacgtcctcactc

SEQ ID NO: 198 - RNA, 98 nt, synthetic construct
  source 1..98: mol_type = other RNA
gttttagagctgtgttgtgaaaacaacacagcgagttaaaataaggctttgtccgtacacaacttgtaaaagtggcacccgattcgggtgcatttttt